SEQUENCE LISTING (2 sequences)

SEQ ID NO: 1 - protein, 182 aa, synthetic construct
  REGION 1..182: note = Synthetic polypeptide
MATGSRTSLLLAFGLLCLPWLQEGSAGSDYKDDDDKGSGSGSLEVLFQGPGITIPRNPGCPNSEDKNFPRTVMVNLNIHNRNTNTNPKRSSDYYNRSTSPWNLHRNEDPERYPSVIWEAQCRHLGCINADGNVDYHMNSVPIQQEILVLRREPPHCPNSFRLEKILVSVGCTCVTPIVHHVQ

SEQ ID NO: 2 - protein, 323 aa, synthetic construct
  REGION 1..323: note = Synthetic polypeptide
MKFLVNVALVFMVVYISYIYALRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGSGGHHHHHHHHHH

(19) United States
(12) Patent Application Publication
(10) Pub. No.: US 2024/0376085 A1
(43) Pub. Date: Nov. 14, 2024

(54) BENZIMIDAZOLES AS MODULATORS OF IL-17

(71) Applicant: Janssen Pharmaceutica NV, Beerse (BE)

(72) Inventors: Charlotte Pooley Deckhut, San Diego, CA (US); Douglas C. Behenna, SanJuan Capistrano, CA (US); Scott Bembenek, San Diego, CA (US); Steven D. Goldberg, San Diego, CA (US); Paul F. Jackson, San Diego, CA (US); John Keith, San Diego, CA (US); Steven A. Loskot, San Diego, CA (US); Connor Martin, San Diego, CA (US); Stefan McCarver, San Diego, CA (US); Steven P. Meduna, Meridian, ID (US); Timothy B. Rhorer, San Diego, CA (US); Amy Y. Shih, San Diego, CA (US); Virginia M. Tanis, Vista, CA (US); Craig R. Woods, San Diego, CA (US); Xiaohua Xue, San Diego, CA (US)

(73) Assignee: Janssen Pharmaceutica NV, Beerse (BE)

(21) Appl. No.: 18/693,310

(22) PCT Filed: Sep. 26, 2022
(86) PCT No.: PCT/US2022/077003

§ 371 Date: Mar. 19, 2024

Related U.S. Application Data
(60) Provisional application No. 63/248,569, filed on Sep. 27, 2021.

(51) Int. Cl. C07D 403/12; A61K 31/4178; A61P 19/02; C07D 413/12

(52) U.S. Cl. CPC C07D 403/12; A61K 31/4178; A61P 19/02; C07D 413/12

(57) ABSTRACT

The present application discloses compounds of Formula (I): (I), or pharmaceutically acceptable salt thereof, wherein R¹, R², R³, and R⁴ are defined in the specification, as well as methods of making and using the compounds disclosed herein for treating or ameliorating an IL-17 mediated syndrome, disorder and/or disease.

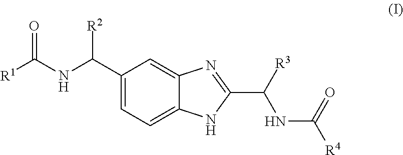

### REFERENCE TO SEQUENCE LISTING SUBMITTED ELECTRONICALLY

This application contains a sequence listing, which is submitted electronically as an ST.26 XML formatted sequence listing with a file name “PRD4157WOPCT1_SL.xml”, creation date of Sep. 7, 2022 and having a size of 4.00 KB. The sequence listing submitted is part of the specification and is herein incorporated by reference in its entirety.

### FIELD

Disclosed herein are benzimidazole compounds, and pharmaceutical compositions thereof, which modulate Interleukin-17A. Also disclosed herein is the therapeutic use of such compounds, for example, in treating and/or ameliorating an IL-17A mediated inflammatory syndrome, disorder, or disease.

### BACKGROUND

Interleukin-17 (“IL-17”), also known as IL-17A and CTLA-8, is produced mainly by CD4+ Th17 cells, and also by other immune cells such as CD8+ T cells, γδ T cells, NK cells, NKT cells, and innate lymphoid cells (ILCs). IL-17A exists as a homodimer (A/A) or as a heterodimer (A/F) with IL-17F and signals through binding to dimeric receptor complex IL-17RA and IL-17RC. IL-17RA is ubiquitously expressed at particularly high levels by haematopoietic cell types, whereas IL-17RC is preferentially expressed by non-haematopoietic cells (Gaffen, S. Structure and signaling in the IL-17 receptor family. Nat. Rev. Immunol. 2009, 9, 556-567). IL-17A/IL-17R signaling induces de novo gene transcription by triggering NF-kB, C/EBP and MAPK pathways through ACT1-TRAF6-TRAF4. It can also stabilize target mRNA transcripts through the ACT1-TRAF2-TRAF5 complex (Amatya N. et al., Trends in Immunology, 2017, 38, 310-322). IL-17A stimulates the release of inflammatory mediators including IL-6, IL-8, G-CSF, TNF-α, and IL-1β that recruit and activate lymphocytes to the site of injury or inflammation and maintain a proinflammatory state.

As discussed below, preclinical and clinical data have demonstrated the significant pathological role of IL-17A in multiple autoimmune and inflammatory diseases.

For psoriasis: IL-17A mRNA and/or protein levels are elevated in the lesional skin and blood of patients with psoriasis and correlate with disease severity. IL-17A acts directly in synergy with other cytokines (such as TNFα, IFNγ or IL-22) on keratinocytes triggering a self-amplifying inflammatory response in the skin and leading to the formation of psoriatic plaques. The blockade of IL-17A by means of antibodies to IL-17A or IL-23 results in complete reversal of the molecular and clinical disease features in majority of psoriasis patients, manifesting the significant role of IL-17A and IL-17-producing T-cells in the immunopathogenesis of psoriasis. (Hawkes et al., Psoriasis Pathogenesis and the Development of Novel, Targeted Immune Therapies. J Allergy Clin Immunol. 2017, 140(3): 645-653). The development and approval of IL-17 monoclonal antibodies such as secukinumab, ixekizumab, and brodalumab and their transformational efficacy for psoriasis have demonstrated IL-17A as a valid target for psoriasis treatments. (Blauvelt A. and Chiricozzi A. The Immunologic Role of IL-17 in Psoriasis and Psoriatic Arthritis Pathogenesis. Clin Rev Allergy Immunol. 2018, 55(3):379-390).

For psoriatic arthritis (PsA): IL-17A is mechanistically relevant to PsA through NFκB activation that triggers transcription of several PsA related genes including the receptor activator of nuclear factor KB ligand (RANKL). RANKL triggers the differentiation of osteoclast precursor cells into activated osteoclasts, resulting in bone resorption and subsequently joint deformity in PsA (Adamopoulos I. and Mellins E. Nature reviews Rheumatology 2015; 11:189-94). PsA joint is enriched for IL-17+CD8+ T cells, and the levels of this T cell subset are correlated with disease activity (Menon B. et al., Arthritis & Rheumatology 2014; 66: 1272-81). Synovial fibroblasts isolated from PsA patients also contain elevated IL-17R expression and secrete increased IL-6, CXCL8 and MMP3 ex vivo compared to osteoarthritis patients. Both secukinumab and ixekizumab are FDA approval drugs for PsA. In matching-adjusted indirect comparison analysis, secukinumab was associated with higher ACR 20/50/70 response rates in patients with active PsA than anti-TNFα antibodies (Mease P. et al., Eur. J. Rheumatol. 2019 Jul. 1; 6(3):113-121; Strand V. et al., J. Comp. Eff. Res. 2019, 8(7):497-510; Nash P. et al., Rheumatol. Ther. 2018, 5(1):99-122). In a recent head-to-head study, ixekizumab was superior to adalimumab in achieving simultaneous improvement of joint and skin disease (ACR50 and PASI100) in patients with PsA and inadequate response to conventional synthetic disease-modifying antirheumatic drug (Mease, P. et al. Ann Rheum Diss 2020; 79:123-131). By hitting the same target, IL-17A small molecule inhibitor compounds may exert similar or better efficacy than biologics considering that small molecules generally have better tissue penetration.

For rheumatoid arthritis (RA): IL-17A has been recognized as critical to the progression of rheumatoid arthritis. “The recognition of IL-17 as a pro-inflammatory T cell derived cytokine, and its abundance within rheumatoid joints, provides the strongest candidate mechanism to date through which T cells can capture and localize macrophage effector functions in rheumatoid arthritis” Stamp, L. et al., Immunol. Cell Biol. 2004, 82(1): 1-9. Moreover, in rheumatoid arthritis IL-17A acts locally on synoviocytes and osteoblasts contributing to synovitis and joint destruction. Robert and Miossec have proposed the use of synovial biopsies and/or biomarkers to precisely identify patients that would respond to IL-17A inhibition. Their work concludes that IL-17 inhibitors should now be considered in the development of precision medicine in RA. (Robert M. and Miossec P., Front. Med., 2019, 5:364).

For Ankylosing Spondylitis (AS): Various studies have reported elevated IL-17A and Th17 and other cells producing IL-17 in AS blood samples (Wendling D. et al., Joint Bone Spine. 2007; 74:304-305; Shen H. et al., Arthritis Rheum. 2009; 60(6):1647-56; Zhang L. et al., PLoS One. 2012; 7 (4):e31000; Jansen D. et al., Rheumatology (Oxford). 2015 April; 54(4): 728-735). In situ analysis of AS spine has revealed increased IL-17A-producing cells in bone of facet (zygapophyseal) joints (Appel H. et al., Arthritis Res. Ther. 2011; 13 (3):R95). Two advanced IL-17A neutralizing antibodies, secukinumab, approved by FDA for AS, and ixekizumab, have demonstrated efficacy over placebo even in anti-TNF inadequate responders. In contrast, anti-IL-23 p40 and p19 biologics failed to demonstrate beneficial effect (Deodhar A. et al., Arthritis Rheumatol. 2019, 71(2):258-270; Baeten D. et al., Ann. Rheum. Dis. 2018, 77(9):1295-1302), indicating the differential underling mechanism along IL-23/IL-17 pathway in AS and providing a strong evidence to support continuing developing IL-17A inhibitors.

For hidradenitis suppurativa (HS): Increased IL-17 and IL-17-producing T helper cells in the skin lesions of HS patients were reported and molecular proteomics and gene expression data indicate that the IL-23/Th17 pathway is upregulated in HS lesions (Schlapbach C. et al., J. Am. Acad. Dermatol. 2011; 65(4):790; Kelly G. et al., British J. Dermatol. 2015 December; 173(6):1431-9; Moran B. et al., J. Invest. Dermatol. 2017; 137(11):2389; Thomi R. et al., JAMA Dermatol. 2018; 154(5):592). Seven of nine (78%) patients with moderate-to-severe HS achieved HiSCR in an open-label pilot-trial with Secukinumab (Prussick L. et al., British J. Dermatol. 2019 September; 181(3):609-611), and more clinical trials with anti-IL-17 mAbs in HS are on-going.

For bullous pemphigold (BP): IL-17 is elevated in the blister fluid and perilesional skin of BP patients. (Le Jan S. et al., J. Invest. Dermatol. 2014; 134 (12):2908-2917.; Chakievska L. J Autoimmun. 2019, 96:104-112). Exome sequencing of BP patients revealed mutations in twelve IL-17-related genes in one third of patients, providing the genetic link between IL-17 pathway and BP (Chakievska L. J Autoimmun. 2019, 96:104-112). In experimental murine BP, IL-17A−/−mice are protected, and anti-IL-17A treatment significantly reduced skin lesions in wild type (Chakievska L. J Autoimmun. 2019, 96:104-112). Ixekizumab Phase 2 of treatment naive and refractory BP patients is on-going (NCT03099538).

For atopic dermatitis (AD): IL-17 was found to be elevated in peripheral blood and lesions in AD patients and Th17 cells infiltrated more markedly in acute than chronic lesions, suggesting its role in acute phase of AD (Koga C. et al., J. Invest. Dermatol. 2008, 128, 2625-2630). Molecular profile analysis from ustekinumab Phase II suggest likely contribution of IL-23/Th17/IL-17 pathway in AD (Khattri S. et al., Exp. Dermatol. 2017 January; 26(1):28-35).

For vitiligo: Many studies in vitiligo patients have demonstrated an increased frequency of Th17 cells and higher levels of IL-17 in both circulation and lesions that positively correlates with disease duration, extent, and activity (Singh R. et al., Autoimmun. Rev 2016, April; 15(4):397-404). Mouse studies demonstrated that depigmentation correlates with greater IL-17 expression/secretion, which modulates vitiligo development (Eby J. et al., Pigment Cell & Melanoma Res. 2014, November; 27(6):1075-85).

For multiple sclerosis (MS): IL-17 expression is increased in PBMCs, cerebrospinal fluid (CSF) as well as in brain lesions and cells from MS patients (Lock, C. et al., Nat. Med. 2002, 8: 500-508; Matusevicius, D. et al., Mult. Scler. 1999, 5: 101-104; Tzartos, J. et al., Am. J. Pathol. 2008, 172: 146-155). IL-17-producing T cells are enriched in active MS lesions (Tzartos, J. et al., Am. J. Pathol. 2008, 172: 146-155; Willing A. et al., J. Immunol. 2018, 200(3):974-982). IL-17A levels were elevated in the CSF of relapsing-remitting MS (RRMS) patients and correlated with the CSF/serum albumin quotient, a measure of blood-brain barrier (BBB) dysfunction, together with in vitro data that IL-17A in combination with IL-6 reduced the expression of tight junction—associated genes and disrupted monolayer integrity in a BBB cell line, highlighting the potential importance of targeting IL-17A in preserving BBB integrity in RRMS (Setiadi A F et al., J Neuroimmunol. 2019, 332:147-154). Secukinumab yielded promising first results in a proof-of-concept study in MS patients (Havrdová, E. et al., J. Neurol. 2016, 263: 1287-1295).

For Asthma: IL-17 expression is increased in the lung, sputum, bronchoalveolar lavage fluid, and sera in patients with asthma, and the severity of airway hyperresponsiveness is positively correlated with IL-17 expression levels. (Chakir J. et al., J. Allergy Clin. Immunol. 2003,111(6):1293-8). IL-17 was reported to be increased in asthmatic airways and induce human bronchial fibroblasts to produce cytokines (Molet S. et al., J. Allergy Clin. Immunol. 2001, 108(3):430-8). Anti-IL-17 antibody modulates airway responsiveness, inflammation, tissue remodeling, and oxidative stress in chronic mouse asthma models (Camargo LdN. et al., Front Immunol. 2018; 8:1835; dos Santos T. et al., Front. Physiol. 2018, 9:1183).

For Chronic Obstructive Pulmonary Disease (COPD): An increase in Th17 cells was observed in patients with COPD compared with current smokers without COPD and healthy subjects, and inverse correlations were found between Th17 cells with lung function (Vargas-Rojas M. et al., Respir. Med. 2011 November; 105(11):1648-54). In three recent human COPD studies, gene expression profile in bronchial epithelia showed that higher IL-17 signature expression is associated with a lack of response to inhaled corticosteroid, suggesting that there is a COPD subgroup that may benefit from IL-17 inhibitor therapy (Christenson S. et al., J. Clin. Invest. 2019; 129(1):169-181).

For Uveitis: IL-17 promotes the release of inflammatory mediators from retinal pigment epithelium cell line, disrupting the retinal pigment epithelium barrier function (Chen Y. et al., PLoS One. 2011; 6:e18139). IL-17 levels were elevated in the serum or aqueous humor of uveitis patients (El-Asrar A. et al., Clin. Immunol. 2011; 139(2):177-84; Jawad S. et al., Ocul. Immunol. Inflamm. 2013; 21(6):434-9; Kuiper J. et al., Am. J. Ophthalmol. 2011; 152(2):177-182.). Anti-IL-17 antibody delayed the onset of ocular inflammation and markedly inhibited the development of experimental autoimmune uveitis in rats (Zhang R. et al., Curr. Eye Res. 2009 April; 34(4):297-303). The analysis of secondary efficacy data from subcutaneous (sc) secukinumab phase 3 trials in uveitis suggested a beneficial effect of secukinumab in reducing the use of concomitant immunosuppressive medication (Dick A. et al., Ophthalmology 2013; 120(4):777-87). Later study of intravenous secukinumab in uveitis demonstrated greater efficacy than sc dosing, suggesting requiring optimal exposure for efficacy and confirming the therapeutic potential of IL-17A inhibition (Letko E. et al., Ophthalmology 2015, 122(5), 939-948). Ustekinumab that blocks IL-23/IL-17 pathway was also reported to successfully treat a noninfectious uveitis patient who had severe concomitant psoriasis and PsA and failed to respond to conventional immune suppressants (Mugheddu C. et al., Dermatol. Ther. 2017 September; 30(5); e12527.).

For multiple myeloma (MM): IL-17A serum levels were significantly higher in MM patients and also in patients with advanced stage compared with healthy subjects (Lemancewicz D. et al., Med. Sci. Monit. 2012; 18 (1): BR54-BR59). Administration of secukinumab in the SCIDhu model of human myeloma weekly for 4 weeks after the first detection of tumor in mice led to a significant inhibition of tumor growth and reduced bone damage compared to isotype control mice (Prabhala R. et al., Leukemia. 2016 February; 30(2): 379-389).

For systemic lupus erythematosus (SLE): Increased serum or plasma levels of IL-17, expansion of IL-17-producing T cells in the peripheral blood, and infiltration of Th17 cells in target organs like the kidneys was observed in SLE patients (Wong C. et al., Lupus. 2000; 9(8):589-593; Wong C. et al., Clinical Immunology. 2008; 127(3):385-393; Zhao X-F. et al., Mol. Biol. Rep. 2010 January; 37(1):81-5; Chen X. et al., J. Clin. Immunol. 2010 March; 30(2):221-5; Xing Q. et al., Rheumatol. Int. 2012 April; 32(4):949-58). Imbalance between Th17 cells and regulatory T (Treg) cells has been observed in SLE patients including quiescent stage (Ma J. et al., Clin. Rheumatol. 2010; 29(11):1251-1258; Dolff S. et al., Clin. Immunol. 2011, 141(2):197-204). Overexpression of IL-17A using adenovirus enhanced the severity of lupus nephritis, while blockade of IL-17A using neutralizing antibody resulted in decreased severity of lupus nephritis (Wen, Z. et al., PLoS One. 2013, 8: e58161). In a phase 2 study, ustekinumab, an anti-IL-12/23 p40 monoclonal antibody blocking IL-23/IL-17 pathway, has demonstrated efficacy in SLE patients (van Vollenhoven R. et al., Lancet 2018; 392: 1330-39). Human expression studies, animal models, and clinical trials indicate that IL-17 blockade may become a promising therapeutic strategy for SLE (Koga T. et al., Expert Rev. Clin. Immunol. 2019, 15 (6) 629-637).

In summary, animal and human studies have shown that IL-17A plays crucial role in pathogenesis of the multiple diseases and/or conditions discussed above. The significance of targeting IL-17A has been demonstrated by the transformational efficacy of injectable IL-17A neutralizing antibodies in patients.

Despite the advances achieved with injectable IL-17A antagonist antibodies, there is a long-felt need for the development of an oral small molecule IL-17A inhibitor as it may broaden treatment options for many patients without access to biologics. In addition, a safe and efficacious small molecule IL-17A inhibitor may offer significant benefits to patients over the injectable IL-17A neutralizing antibodies such as convenient dosing regimens and cost savings, which in turn may provide effective long-term disease management.

However, the development of an oral small molecule treatment has remained challenging. For example, no oral small molecule IL-17A inhibitor has progressed into late-stage clinical trials yet, and only two oral small molecule IL-17A inhibitors have progressed into phase I clinical trials (NCT04586920 and NCT04883333) as of Sep. 28, 2021. Additionally, as of December 2021, one of these clinical trials (NCT04586920) was suspended due to safety review. Accordingly, there is a need for new small molecule IL-17A modulators (e.g., inhibitors).

### SUMMARY

The present application discloses a compound of Formula (I):

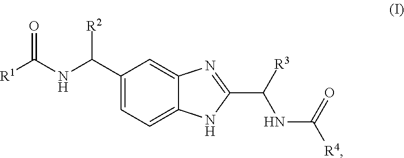

or a pharmaceutically acceptable salt thereof, wherein:
R¹ is —C₍₁₋₆₎alkyl, —C₍₁₋₃₎alkyl-C₍₃₋₆₎cycloalkyl, or —C₍₁₋₃₎alkyl-C₍₅₋₁₀₎polycycloalkyl, each of which is unsubstituted or substituted with one to six R^{1a} groups;
each R^{1a} independently for each occurrence is fluorine, —C₍₁₋₃₎alkyl, —C₍₃₋₅₎cycloalkyl, —CN, —OH, —O—C₍₁₋₃₎alkyl, or —O—C₍₃₋₄₎cycloalkyl, wherein the —C₍₁₋₃₎alkyl, —C₍₃₋₅₎cycloalkyl, —O—C₍₁₋₃₎alkyl, and —O—C₍₃₋₄₎cycloalkyl groups are unsubstituted or substituted with one to three fluorine atoms;
R² is —C₍₁₋₆₎alkyl, —C₍₃₋₅₎cycloalkyl, —C₍₁₋₃₎alkyl-C₍₃₋₅₎cycloalkyl, C₍₁₋₃₎alkyl-O—C₍₁₋₃₎alkyl, C₍₁₋₃₎alkyl-O—C₍₃₋₅₎cycloalkyl or 4- to 6-membered heterocyclyl, each of which is unsubstituted or substituted with one to six R^{2a} groups;
each R^{2a} independently for each occurrence is fluorine, —C₍₁₋₃₎alkyl, —C₍₃₋₅₎cycloalkyl, —CN, —OH, —O—C₍₁₋₃₎alkyl, or —O—C₍₃₋₄₎cycloalkyl, wherein the —C₍₁₋₃₎alkyl, C₍₃₋₅₎cycloalkyl, —O—C₍₁₋₃₎alkyl, and —O—C₍₃₋₄₎cycloalkyl groups are unsubstituted or substituted with one to three fluorine atoms;
R³ is —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, or —C₍₃₋₄₎cycloalkylC₍₁₋₃₎alkyl, wherein the —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, and —C₍₃₋₄₎cycloalkylC₍₁₋₃₎alkyl are unsubstituted or substituted with one to four R^{3a} groups;
each R^{3a} independently for each occurrence is fluorine, —CH₃, —CH₂F, —CHF₂, —CF₃, —O—C₍₁₋₃₎alkyl, —OH, or oxo;
R⁴ is —C₍₁₋₆₎alkyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl,
wherein the C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups,
wherein the phenyl is unsubstituted or substituted with one to three R^{4b} groups,
wherein the 5-membered heteroaryl is unsubstituted or substituted with one to three R^{4c} groups;
each R^{4a} independently for each occurrence is fluorine, —C₍₁₋₃₎alkyl, or —CN, wherein the —C₍₁₋₃₎alkyl is unsubstituted or substituted with one to three fluorine atoms;
each R^{4b} independently for each occurrence is fluorine or —CN;
each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl, are unsubstituted or substituted with one to six substituents independently selected from the group consisting of fluorine, —OH, and —CN;
alternatively, two R^{4c} groups attached to vicinal atoms on the same ring can be combined with the atoms to which they are attached to form a C₍₃₋₆₎cycloalkyl or a 3- to 6-membered heterocyclyl.

In some embodiments, disclosed herein is a pharmaceutical composition comprising a compound of Formula (I), or pharmaceutically acceptable salt thereof, and a pharmaceutically acceptable carrier.

Also described herein is a method for treating and/or ameliorating an IL-17A mediated inflammatory syndrome, disorder, or disease (e.g., psoriasis, psoriatic arthritis, rheumatoid arthritis, ankylosing spondylitis, etc.) comprising administering to a subject in need thereof a therapeutically effective amount of a compound of Formula (I), or a pharmaceutically acceptable salt thereof.

In some embodiments, disclosed herein is the use of a therapeutically effective amount of compound of Formula (I), or a pharmaceutically acceptable salt thereof, for treating and/or ameliorating an IL-17A mediated inflammatory syndrome, disorder, or disease (e.g., psoriasis, psoriatic arthritis, rheumatoid arthritis, ankylosing spondylitis, etc.).

In some embodiments, disclosed herein is the use of a compound of Formula (I), or pharmaceutically acceptable salt thereof, in the manufacture of a medicament for treating and/or ameliorating an IL-17A mediated inflammatory syndrome, disorder, or disease (e.g., psoriasis, psoriatic arthritis, rheumatoid arthritis, ankylosing spondylitis, etc.).

In some embodiments, provided herein are processes and intermediates disclosed herein that are useful for preparing a compound of Formula (I) or pharmaceutically acceptable salts thereof.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The term “administering” with respect to the methods of the invention, means a method for therapeutically or prophylactically preventing, treating or ameliorating a syndrome, disorder or disease as described herein by using a compound of Formula (I), or pharmaceutically acceptable salt thereof, composition thereof, or medicament thereof. Such methods include administering a therapeutically effective amount of a compound of Formula (I), or pharmaceutically acceptable salt thereof, composition thereof, or medicament thereof, at different times during the course of a therapy or concurrently or sequentially as a combination therapy.

The term “subject” refers to a patient, which may be an animal, preferably a mammal, most preferably a human, whom will be or has been treated by a method according to an embodiment of the application. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

The term “therapeutically effective amount” or “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

As used herein, “IL-17” or “IL-17A” refers to interleukin 17A. It is also named IL17, CTLA8, CTLA-8. Interleukin 17A is a pro-inflammatory cytokine. This cytokine is produced by a group of immune cells in response to their stimulation. An exemplary amino acid sequence of human IL-17 is represented in GenBank Accession No. NP_002181.1, which can be encoded by a nucleic acid sequence such as that of GenBank Accession No. NM_002190.3.

The term “modulator” as used herein refers to any agents or molecules that can bind to IL-17, including small molecule compounds.

“Active moiety” refers to a molecule or ion responsible for a physiological or pharmacological action. A compound of formula (I), as exemplified in the Examples and also described herein, is an active moiety.

As used herein, the term “composition” is intended to encompass a product comprising the specified ingredients in the specified amounts, as well as any product which results, directly or indirectly, from combinations of the specified ingredients in the specified amounts.

As used herein, the term “treat,” “treating,” or “treatment” of any disease, condition, syndrome or disorder refers, in one embodiment, to ameliorating the disease, condition, syndrome or disorder (i.e., slowing or arresting or reducing the development of the disease or at least one of the clinical symptoms thereof). In another embodiment, “treat”, “treating”, or “treatment” refers to alleviating or ameliorating at least one physiological or biochemical parameter associated with or causative of the disease, condition, syndrome or disorder, including those which may not be discernible by the patient. In a further embodiment, “treat,” “treating,” or “treatment” refers to modulating the disease, condition, syndrome or disorder either physically (e.g. stabilization of a discernible symptom), physiologically, (e.g. stabilization of a physical parameter), or both. In yet another embodiment, “treat,” “treating,” or “treatment” refers to preventing or delaying the onset or development or progression of the disease, condition, syndrome or disorder.

As used herein, the term “QD” means once daily.

As used herein, the term “BID” means twice daily.

The term “alkyl” is a straight or branched saturated hydrocarbon having the designated number of carbon atoms. For example, an alkyl group can have 1 to 12 carbon atoms (i.e., (C₁-C₁₂)alkyl) or 1 to 6 carbon atoms (i.e., (C₁-C₆)alkyl). Examples of alkyl groups include, but are not limited to, methyl (Me, —CH₃), ethyl (Et, —CH₂CH₃), 1-propyl (n-Pr, n-propyl, —CH₂CH₂CH₃), isopropyl (i-Pr, i-propyl, —CH(CH₃)₂), 1-butyl (n-Bu, n-butyl, —CH₂CH₂CH₂CH₃), 2-butyl (s-Bu, s-butyl, —CH(CH₃)CH₂CH₃), tert-butyl (t-Bu, t-butyl, —CH(CH₃)₃), 1-pentyl (n-pentyl, —CH₂CH₂CH₂CH₂CH₃), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃)CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH₂)₁₀CH₃), and dodecyl (—(CH₂)₁₁CH₃). Any alkyl group may suitably be unsubstituted or substituted as described herein.

The term “C_{(a-b)}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “heterocycle” or “heterocyclyl” refers to a single saturated or partially unsaturated ring that has at least one atom other than carbon in the ring, wherein the atom is selected from the group consisting of oxygen, nitrogen and sulfur. Exemplary heterocycles include, but are not limited to oxetanyl, aziridinyl, azetidinyl, pyrrolidinyl, piperidinyl, piperazinyl, morpholinyl, tetrahydropyranyl, tetrahydrofuranyl, and thiomorpholinyl.

The term “polyheterocycle” or “polyheterocyclyl” refers to a ring system comprising two or more saturated or partially unsaturated rings, wherein at least one of the rings comprises at least one atom other than carbon, and wherein the atom is selected from the group consisting of oxygen, nitrogen and sulfur. Polyheterocyclyl groups may, for example, be bicylcic, tricyclic, tetracyclic, or pentacyclic. The multiple rings of the polyheterocyclyl ring system may be in a fused, spirocyclic, or bridged configuration.

The term “cycloalkyl” refers to a single saturated or partially unsaturated all carbon ring having the specified number of carbon atoms (e.g., C₍₃₋₈₎cycloalkyl). Exemplary cycloalkyls include, but are not limited to, cyclopropyl, cyclobutyl, cyclopentyl, cyclohexyl, cycloheptyl, and cyclooctyl. Unless otherwise stated specifically in the specification, a cycloalkyl group may be unsubstituted or substituted.

The term “polycycloalkyl” refers to a saturated or partially unsaturated all carbon ring system comprising two or more rings having the specified number of carbon atoms (e.g., C₍₅₋₈₎polycycloalkyl). Polycycloalkyl groups may, for example, be bicylcic, tricyclic, tetracyclic, or pentacyclic. The multiple rings of the polycycloalkyl ring system may be in a fused, spirocyclic, or bridged configuration. Some polycycloalkyl groups may exist as fused polycycloalkyls, wherein two cycloalkyl rings share a carbon-carbon bond; for example and without limitation, fused polycycloalkyl groups include:

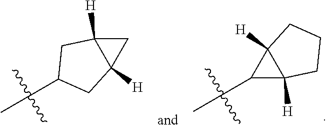 [SMILES: *C1C[C@@H]2C[C@@H]2C1 ; *C1[C@H]2CCC[C@@H]12 ; "and"]

Some polycycloalkyl groups may exist as spiro polycycloalkyls, wherein two cycloalkyl rings are fused through a single carbon atom; for example and without limitation, an example of a spiropentyl group is

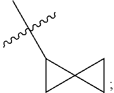 [SMILES: *C1CC12CC2]

for example and without limitation, examples of spirohexyl groups include

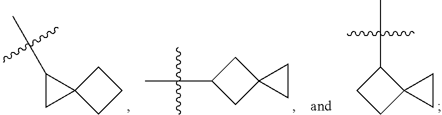 [SMILES: *C1CC12CCC2 ; *C1CC2(CC2)C1 ; *C1CCC12CC2]

for example and without limitation examples of spiroheptyl groups include

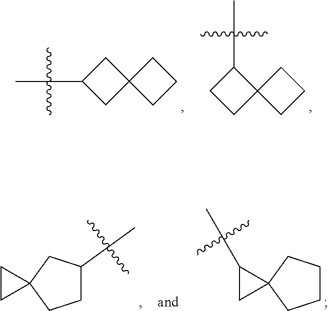

for example and without limitation examples of spirooctyl groups include

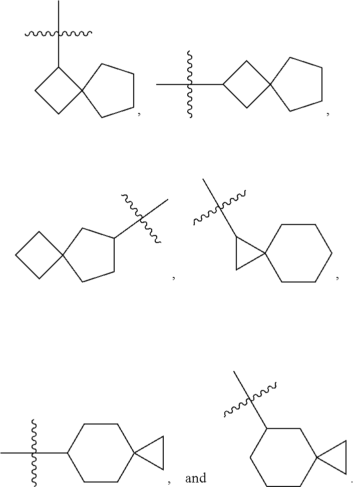 [SMILES: *C1CCC12CCCC2 ; *C1CC2(CCCC2)C1 ; *C1CCC2(CCC2)C1 ; *C1CC12CCCCC2 ; *C1CCC2(CC1)CC2 ; *C1CCCC2(CC2)C1]

Bicyclic polycycloalkyl ring systems also include

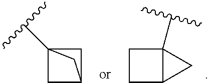 [SMILES: *C12CC(C1)C2 ; *C12CCC1C2]

Unless otherwise stated specifically in the specification, a polycycloalkyl group may be unsubstituted or substituted.

The term “heteroaryl” refers to a single aromatic ring that has at least one atom other than carbon in the ring, wherein the atom is selected from the group consisting of oxygen, nitrogen and sulfur. The term “heteroaryl,” for example, includes single aromatic rings of from 1 to 6 carbon atoms and 1 to 4 heteroatoms selected from the group consisting of oxygen, nitrogen and sulfur. Exemplary heteroaryl ring systems include but are not limited to pyridinyl, pyrimidinyl, pyrazinyl, pyridazinyl, pyrimidinyl, pyrazolyl, oxazolyl, oxadiazolyl, isoxazolyl, triazolyl, imidazolyl, tetrazolyl, thienyl, thiazolyl, isothiazolyl, thiadiazolyl, or furyl.

Where the compounds of Formula (I) disclosed herein have at least one stereo center, they may accordingly exist as enantiomers or diastereomers. It is to be understood that all such isomers and mixtures thereof are encompassed within the scope of the present invention.

“Diastereoisomers” are stereoisomers that have at least two asymmetric atoms, but which are not mirror images of each other.

“Enantiomers” are a pair of stereoisomers that are non-superimposable mirror images of each other. A “racemic” mixture is a 1:1 mixture of a pair of enantiomers. A “scalemic” mixture of enantiomers is mixture of enantiomers at a ratio other than 1:1.

Where the processes for the preparation of the compounds according to the invention give rise to mixture of stereoisomers, these isomers may be separated by conventional techniques such as preparative chromatography. The compounds may be prepared in racemic form, a scalemic mixture, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral column vial HPLC or SFC. In some instances rotamers of compounds may exist which are observable by ¹H NMR leading to complex multiplets and peak integration in the ¹H NMR spectrum.

The absolute stereochemistry is specified according to the Cahn-Ingold-Prelog R-S system. Chiral centers, of which the absolute configurations are known, are labelled by prefixes R and S, assigned by the standard sequence-rule procedure, and preceded when necessary by the appropriate locants (Pure & Appl. Chem. 45, 1976, 11-30). Certain examples contain chemical structures that are depicted or labelled as an (R*) or (S*). When (R*) or (S*) is used in the name of a compound or in the chemical representation of the compound, it is intended to convey that the compound is a pure single isomer at that stereocenter; however, absolute configuration of that stereocenter has not been established. Thus, a compound designated as (R*) refers to a compound that is a pure single isomer at that stereocenter with an absolute configuration of either (R) or (S), and a compound designated as (S*) refers to a compound that is a pure single isomer at that stereocenter with an absolute configuration of either (R) or (S). For example, 2-(cyclopropylmethyl)-N—((S*)-4,4,4-trifluoro-3,3-dimethyl-1-(5-((R)-1-(4,4,4-trifluorobutanamido)ethyl)-1H-benzo[d]imidazol-2-yl)butyl)-2H-1,2,3-triazole-4-carboxamide:

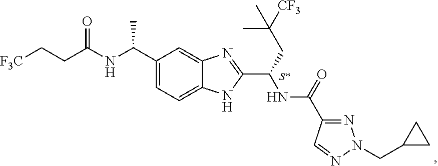 [SMILES: C[C@@H](NC(=O)CCC(F)(F)F)c1ccc2[nH]c([C@H](CC(C)(C)C(F)(F)F)NC(=O)c3cnn(CC4CC4)n3)nc2c1]

refers to a compound that is either:

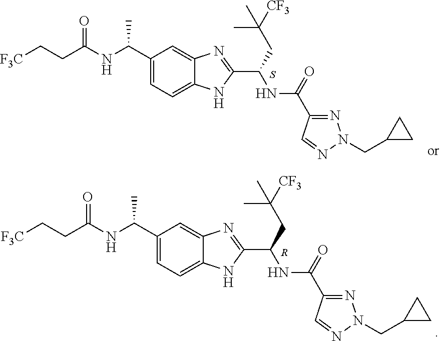

Pseudoasymmetric stereogenic centers are treated in the same way as chiral centers, but are given lower-case symbols, r or s (Angew. Chem. Int. Ed. Engl. 1982, 21, 567-583).

Where a chiral center exists on a given compound but the sterochemistry is not specified, any configuration of the unspecified stereocenter is envisioned.

In one embodiment, the disclosed compounds may exist as tautomers. All tautomers are included within the scope of the compounds presented herein.

During any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Furthermore, it is intended that within the scope of the present invention, any element, in particular when mentioned in relation to a compound of Formula (I), or pharmaceutically acceptable salt thereof, shall comprise all isotopes and isotopic mixtures of said element, either naturally occurring or synthetically produced, either with natural abundance or in an isotopically enriched form. For example, a reference to hydrogen includes within its scope ¹H, ²H (D), and ³H (T). In some embodiments, the compounds described herein include a ²H (i.e., deuterium) isotope. By way of example, the group denoted —C₍₁₋₆₎alkyl includes not only —CH₃, but also CD₃; not only CH₂CH₃, but also CD₂CD₃. Similarly, references to carbon and oxygen include within their scope respectively ¹²C, ¹³C, and ¹⁴C and ¹⁵O, ¹⁶O, ¹⁷O, and ¹⁸O. The isotopes may be radioactive or non-radioactive. Radiolabelled compounds of Formula (I) may include a radioactive isotope selected from the group comprising ³H, ¹¹C, ¹⁸F, ³⁵S, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

### Compounds of Formula I

The present disclosure provides a compound of Formula (I):

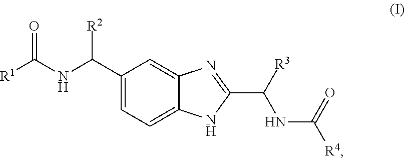

or a pharmaceutically acceptable salt thereof, wherein:
R¹ is —C₍₁₋₆₎)alkyl, —C₍₁₋₃₎alkyl-C₍₃₋₆₎cycloalkyl, or —C₍₁₋₃₎alkyl-C₍₅₋₁₀₎polycycloalkyl, each of which is unsubstituted or substituted with one to six R^{1a} groups;
each R^{1a} independently for each occurrence is fluorine, —C₍₁₋₃₎alkyl, —C₃₋₅₎cycloalkyl, —CN, —OH, —O—C₍₁₋₃₎alkyl, or —O—C₍₃₋₄₎cycloalkyl, wherein the —C₍₁₋₃₎alkyl, —C₍₃₋₅₎cycloalkyl, —O—C₍₁₋₃₎alkyl, and —O—C₍₃₋₄₎cycloalkyl groups are unsubstituted or substituted with one to three fluorine atoms;
R² is —C₍₁₋₆₎)alkyl, —C₍₃₋₅₎cycloalkyl, —C₍₁₋₃₎alkyl-C₍₃₋₅₎cycloalkyl, —C₍₁₋₃₎alkyl-O—C₍₁₋₃₎alkyl, C₍₁₋₃₎alkyl-O—C₍₃₋₅₎cycloalkyl, or 4- to 6-membered heterocyclyl, each of which is unsubstituted or substituted with one to six R^{2a} groups;
each R^{2a} independently for each occurrence is fluorine, —C₍₁₋₃₎alkyl, —C₍₃₋₅₎cycloalkyl, —CN, —OH, —O—C₍₁₋₃₎alkyl, or —O—C₍₃₋₄₎cycloalkyl, wherein the —C₍₁₋₃₎alkyl, C₍₃₋₅₎cycloalkyl, —O—C₍₁₋₃₎alkyl, and —O—C₍₃₋₄₎cycloalkyl groups are unsubstituted or substituted with one to three fluorine atoms;
R³ is —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, or —C₍₃₋₄₎cycloalkylC₍₁₋₃₎alkyl, wherein the —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, and —C₍₃₋₄₎cycloalkylC₍₁₋₃₎alkyl are unsubstituted or substituted with one to four R^{3a} groups;
each R^{3a} independently for each occurrence is fluorine, —CH₃, —CH₂F, —CHF₂, —CF₃, —O—C₍₁₋₃₎alkyl, —OH, or oxo;
R⁴ is —C₍₁₋₆₎)alkyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl,
wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups,
wherein the phenyl is unsubstituted or substituted with one to three R^{4b} groups,
wherein the 5-membered heteroaryl is unsubstituted or substituted with one to three R^{4c} groups;
each R^{4a} independently for each occurrence is fluorine, —C₍₁₋₃₎alkyl, or —CN, wherein the —C₍₁₋₃₎alkyl is unsubstituted or substituted with one to three fluorine atoms;
each R^{4b} independently for each occurrence is fluorine or —CN;
each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl are unsubstituted or substituted with one to six substituents independently selected from the group consisting of fluorine, —OH, and —CN; alternatively, two R^{4c} groups attached to vicinal atoms on the same ring can be combined with the atoms to which they are attached to form a C₍₃₋₆₎cycloalkyl or a 3- to 6-membered heterocyclyl.

The present disclosure provides a compound of Formula (I):

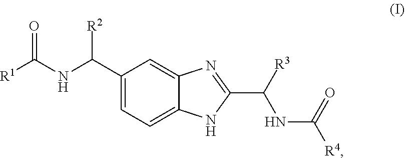

or a pharmaceutically acceptable salt thereof, wherein:
R¹ is —C₍₁₋₆₎)alkyl, —C₍₁₋₃₎alkyl-C₍₃₋₆₎cycloalkyl, or —C₍₁₋₃₎alkyl-C₍₅₋₁₀₎polycycloalkyl, each of which is unsubstituted or substituted with one to six R^{1a} groups;
each R^{1a} independently for each occurrence is fluorine, —C₍₁₋₃₎alkyl, —C₍₃₋₅₎cycloalkyl, —CN, —OH, —O—C₍₁₋₃₎alkyl, or —O—C₍₃₋₄₎cycloalkyl, wherein the —C₍₁₋₃₎alkyl, —C₍₃₋₅₎cycloalkyl, —O—C₍₁₋₃₎alkyl, and —O—C₍₃₋₄₎cycloalkyl groups are unsubstituted or substituted with one to three fluorine atoms;
R² is —C₍₁₋₆₎)alkyl, —C₍₃₋₅₎cycloalkyl, —C₍₁₋₃₎alkyl-C₍₃₋₅₎cycloalkyl, —C₍₁₋₃₎alkyl-O—C₍₁₋₃₎alkyl, or 4- to 6-membered heterocyclyl, each of which is unsubstituted or substituted with one to six R² groups;
each R^{2a} independently for each occurrence is fluorine, —C₍₁₋₃₎alkyl, —C₍₃₋₅₎cycloalkyl, —CN, —OH, —O—C₍₁₋₃₎alkyl, or —O—C₍₃₋₄₎cycloalkyl, wherein the —C₍₁₋₃₎alkyl, C₍₃₋₅₎cycloalkyl, —O—C₍₁₋₃₎alkyl, and —O—C₍₃₋₄₎cycloalkyl groups are unsubstituted or substituted with one to three fluorine atoms;
R³ is —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, or —C₍₃₋₄₎cycloalkylC₍₁₋₃₎alkyl, wherein the —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, and —C₍₃₋₄₎cycloalkylC₍₁₋₃₎alkyl are unsubstituted or substituted with one to four R^{3a} groups;
each R^{3a} independently for each occurrence is fluorine, —CH₃, —CH₂F, —CHF₂, —CF₃, —O—C₍₁₋₃₎alkyl, —OH, or oxo;
R⁴ is —C₍₁₋₆₎)alkyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl,
wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups,
wherein the phenyl is unsubstituted or substituted with one to three R^{4b} groups,
wherein the 5-membered heteroaryl is unsubstituted or substituted with one to three R^{4c} groups;
each R^{4a} independently for each occurrence is fluorine, —C₍₁₋₃₎alkyl, or —CN, wherein the —C₍₁₋₃₎alkyl is unsubstituted or substituted with one to three fluorine atoms;
each R^{4b} independently for each occurrence is fluorine or —CN;
each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl are unsubstituted or substituted with one to six substituents independently selected from the group consisting of fluorine, —OH, and —CN;
alternatively, two R^{4c} groups attached to vicinal atoms on the same ring can be combined with the atoms to which they are attached to form a C₍₃₋₆₎cycloalkyl or a 3- to 6-membered heterocyclyl.

In some embodiments, disclosed herein is a compound of Formula (I), or a pharmaceutically acceptable salt thereof, wherein:
R¹ is

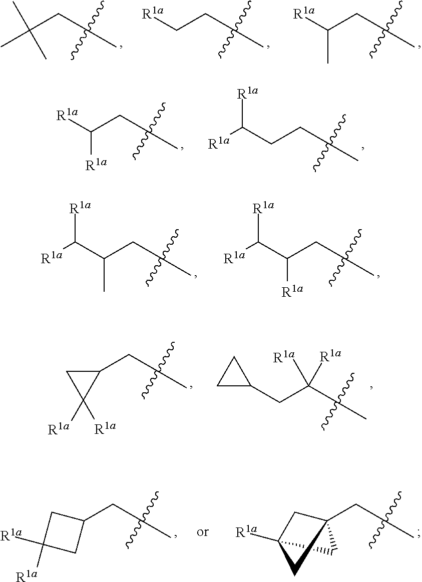

each R^{1a} independently for each occurrence is fluorine, —C₍₁₋₃₎alkyl, or —C₍₃₋₅₎cycloalkyl, wherein the —C₍₁₋₃₎alkyl and —C₍₃₋₅₎cycloalkyl groups are unsubstituted or substituted with one to three fluorine atoms;
R² is —C₍₁₋₆₎)alkyl, —C₍₃₋₅₎cycloalkyl, —C₍₁₋₃₎alkyl-C₍₃₋₅₎cycloalkyl, —C₍₁₋₃₎alkyl-O—C₍₁₋₃₎alkyl, C₍₁₋₃₎alkyl-O— C₍₃₋₅₎cycloalkyl, or 4- to 6-membered heterocyclyl, each of which is unsubstituted or substituted with one to six R^{2a} groups;
each R^{2a} independently for each occurrence is fluorine or —CN;
R³ is —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, or —C₍₃₋₄₎cycloalkylC₍₁₋₃₎alkyl, wherein the —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, and —C₍₃₋₄₎cycloalkylC₍₁₋₃₎alkyl are unsubstituted or substituted with one to four R^{3a} groups;
each R^{3a} independently for each occurrence is fluorine, —CH₃, —CH₂F, —CHF₂, —CF₃, —O—C₍₁₋₃₎alkyl, —OH, or oxo;
R⁴ is —C₍₁₋₆₎)alkyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl,
wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups,
wherein the phenyl is unsubstituted or substituted with one to three R^{4b} groups,
wherein the 5-membered heteroaryl is unsubstituted or substituted with one to three R^{4c} groups;
each R^{4a} independently for each occurrence is fluorine, CH₃, CH₂F, CHF₂, CF₃, or —CN;
each R^{4b} independently for each occurrence is fluorine or —CN;
each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl, are unsubstituted or substituted with one to six substituents independently selected from the group consisting of fluorine, —OH, and —CN;
alternatively, two R^{4c} groups attached to vicinal atoms on the same ring can be combined with the atoms to which they are attached to form a C₍₃₋₆₎cycloalkyl or a 3- to 6-membered heterocyclyl.

In some embodiments, disclosed herein is a compound of Formula (I), or a pharmaceutically acceptable salt thereof, wherein:
R¹ is

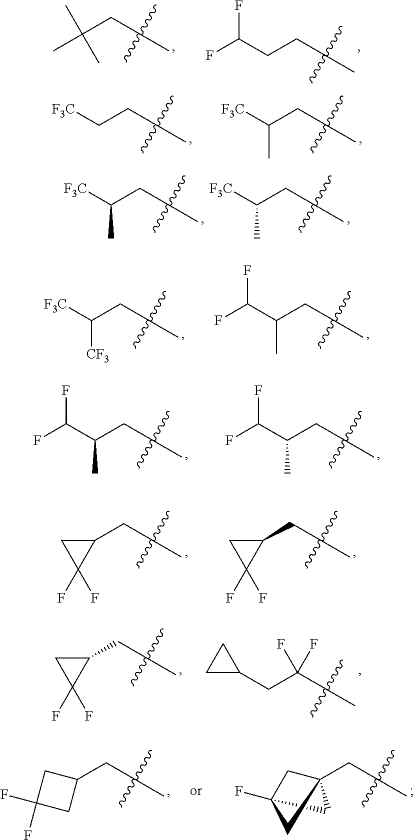

R² is

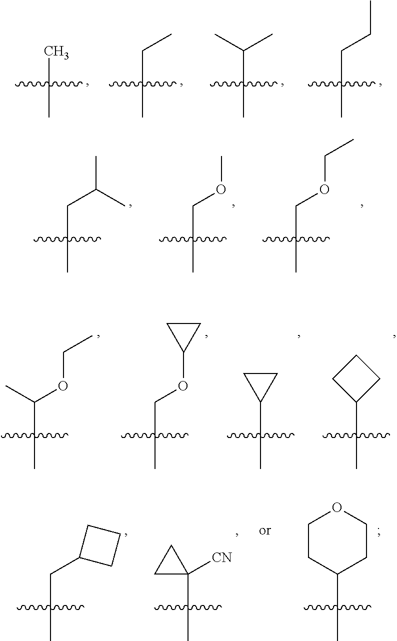

R³ is —C₍₃₋₆₎alkyl, —C₍₃₋₆₎cycloalkyl, —C₍₅₋₁₀₎polycycloalkyl, 3- to 6-membered heterocyclyl —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, or —C₍₃₋₄₎cycloalkylC₍₁₋₃₎alkyl, wherein the —C₍₃₋₆₎alkyl, —C₍₃₋₆₎cycloalkyl, —C₍₅₋₁₀₎polycycloalkyl, 3- to 6-membered heterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, and —C₍₃₋₄₎cycloalkylC₍₁₋₃₎alkyl are unsubstituted or substituted with one to four R^{3a} groups;
each R^{3a} independently for each occurrence is fluorine, —CH₃, —CH₂F, —CHF₂, —CF₃, —O—C₍₁₋₃₎alkyl, —OH, or oxo;
R⁴ is —C₍₁₋₆₎)alkyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl,
wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups,
wherein the phenyl is unsubstituted or substituted with one to three R^{4b} groups,
wherein the 5-membered heteroaryl is unsubstituted or substituted with one to three R^{4c} groups;
each R^{4a} independently for each occurrence is fluorine, CH₃, CH₂F, CHF₂, CF₃, or —CN;
each R^{4b} independently for each occurrence is fluorine or —CN;
each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl are unsubstituted or substituted with one to six substituents independently selected from the group consisting of fluorine, —OH, and —CN;
alternatively, two R^{4c} groups attached to vicinal atoms on the same ring can be combined with the atoms to which they are attached to form a C₍₃₋₆₎cycloalkyl or a 3- to 6-membered heterocyclyl.

In some embodiments, disclosed herein is a compound of Formula (I), or a pharmaceutically acceptable salt thereof, wherein:
R¹ is

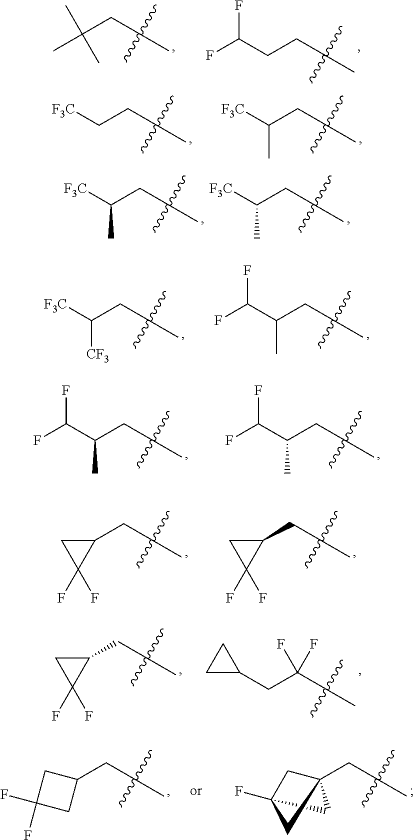

R² is

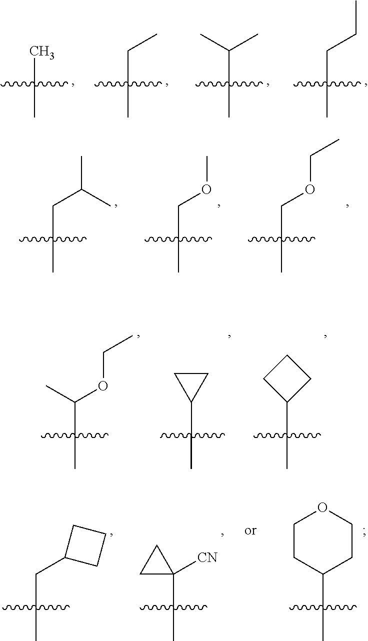

R³ is —C₍₃₋₆₎alkyl, —C₍₃₋₆₎cycloalkyl, —C₍₅₋₁₀₎polycycloalkyl, tetrahydropyranyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, or —C₍₃₋₄₎cycloalkylC₍₁₋₃₎alkyl, wherein the —C₍₃₋₆₎alkyl, —C₍₃₋₆₎cycloalkyl, —C₍₅₋₁₀₎polycycloalkyl, tetrahydropyranyl, —C₍₁₋₂₎alkyl-O—C₍₁₋₅₎alkyl, —C₍₁₋₂₎alkylC₍₃₋₆₎cycloalkyl, C₍₁₋₂₎alkyl-O—C₍₃₋₆₎cycloalkyl, —C₍₁₋₂₎alkyl-C₍₅₋₁₀₎polycycloalkyl, and —C₍₃₋₄₎cycloalkylC₍₁₋₃₎alkyl are unsubstituted or substituted with one to three R^{3a} groups;
each R^{3a} independently for each occurrence is fluorine, —CH₃, —CH₂F, —CHF₂, or —CF₃;
R⁴ is isopropyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl, wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups, wherein the phenyl is unsubstituted or substituted with one to three R^{4b} groups, and wherein the 5-membered heteroaryl is unsubstituted or substituted with one to three R^{4c} groups;
each R^{4a} independently for each occurrence is fluorine, —CH₃, CH₂F, —CHF₂, —CF₃, or —CN;
each R^{4b} independently for each occurrence is fluorine or —CN;
each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎-alkyl-O—C₍₃₋₆₎-cycloalkyl, and —O—C₍₁₋₃₎-alkyl are unsubstituted or substituted with one to six substituents independently selected from the group consisting of fluorine, —OH, and —CN;
alternatively, two R^{4c} groups attached to vicinal atoms on the same ring can be combined with the atoms to which they are attached to form a C₍₃₋₆₎cycloalkyl or a 3- to 6-membered heterocyclyl.

In some embodiments, disclosed herein is a compound of Formula (I), or a pharmaceutically acceptable salt thereof, wherein R¹ is —C₍₁₋₆₎)alkyl, —C₍₁₋₃₎alkyl-C₍₃₋₆₎cycloalkyl, or —C₍₁₋₃₎alkyl-C₍₅₋₁₀₎polycycloalkyl, each of which is substituted with one to six R^{1a} groups, or R is:

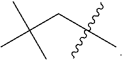 [SMILES: *CC(C)(C)C]

In some embodiments, R¹ is —C₍₁₋₆₎)alkyl, —C₍₁₋₃₎alkyl-C₍₃₋₆₎cycloalkyl, or —C₍₁₋₃₎alkyl-C₍₅₋₁₀₎polycycloalkyl, each of which is substituted with one to six fluorine atoms, or R¹ is:

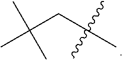 [SMILES: *CC(C)(C)C]

In some embodiments, R¹ is —C₍₁₋₄₎alkyl, —CH₂—C₍₃₋₄₎cycloalkyl, or —CH₂—C₍₅₋₈₎polycycloalkyl, each of which is unsubstituted or substituted with one to six fluorine atoms.

In some embodiments, R¹ is

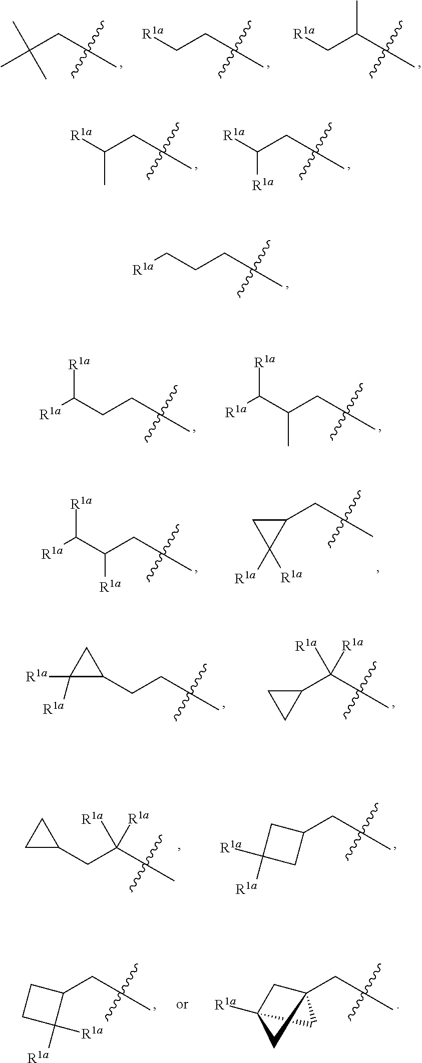

In some embodiments, R¹ is

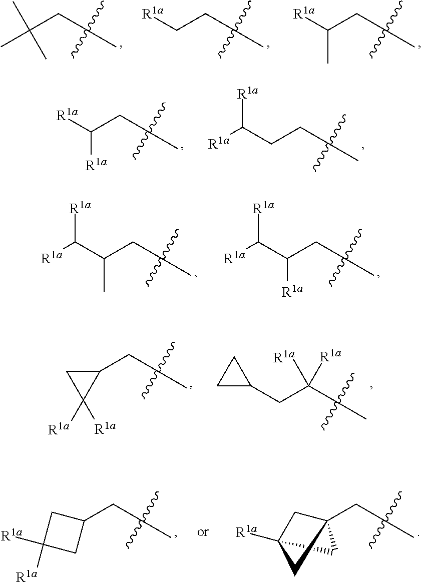

In some embodiments, each R^{1a} independently for each occurrence is fluorine, —CH₂F, —CHF₂, or —CF₃.

In some embodiments, R¹ is:

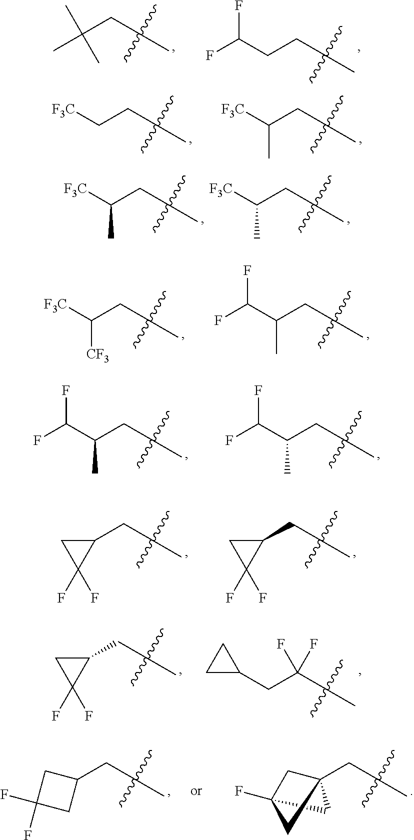

In some embodiments, R¹ is:

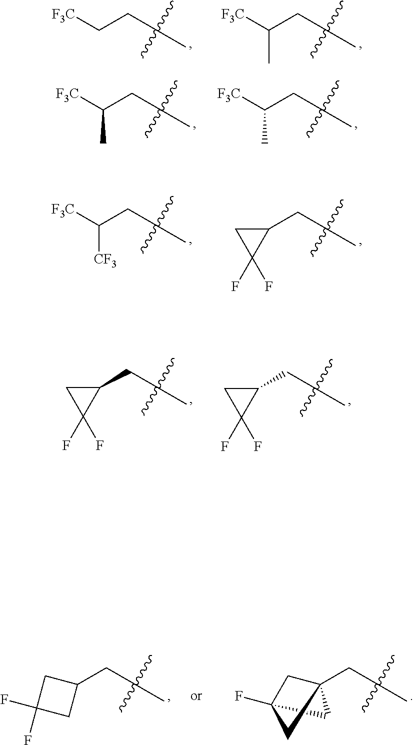

In some embodiments, R¹ is:

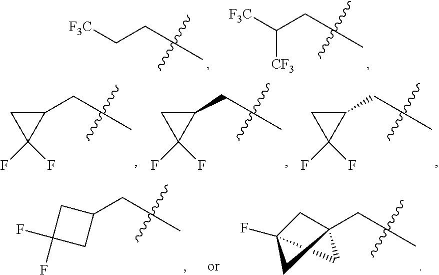

In some embodiments, disclosed herein is a compound of Formula (I), or a pharmaceutically acceptable salt thereof, wherein R² is —C₍₁₋₆₎alkyl, —C₍₃₋₅₎cycloalkyl, —C₍₁₋₃₎alkyl-C₍₃₋₅₎cycloalkyl, —C₍₁₋₃₎alkyl-O—C₍₁₋₃₎alkyl, or 4- to 6-membered heterocyclyl, each of which is unsubstituted or substituted with one to six substituents selected from the group consisting of fluorine and —CN.

In some embodiments, R² is —C₍₁₋₆₎)alkyl, —C₍₃₋₅₎cycloalkyl, —C₍₁₋₃₎alkyl-C₍₃₋₅₎cycloalkyl, —C₍₁₋₃₎alkyl-O—C₍₁₋₃₎alkyl, or 4- to 6-membered heterocyclyl, wherein the C₍₃₋₅₎cycloalkyl is unsubstituted or substituted with one —CN.

In some embodiments, R² is —C₍₁₋₄₎alkyl, —C₍₃₋₄₎cycloalkyl, —CH₂—C₍₃₋₄₎cycloalkyl, —C₍₁₋₂₎alkyl-O—C₍₁₋₂₎alkyl, or tetrahydropyranyl, wherein the —C₍₃₋₄₎cycloalkyl is unsubstituted or substituted with one —CN.

In some embodiments, R² is —C₍₁₋₃₎alkyl, cyclopropyl, cyclobutyl, or C₍₁₋₂₎alkyl-O—C₍₁₋₂₎alkyl, wherein the cyclopropyl is unsubstituted or substituted with one —CN.

In some embodiments, R² is

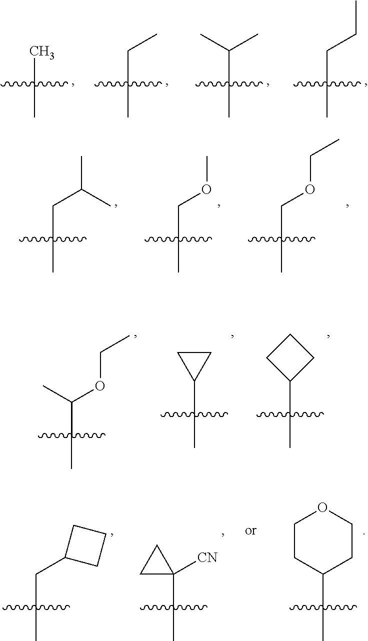

In some embodiments, R² is

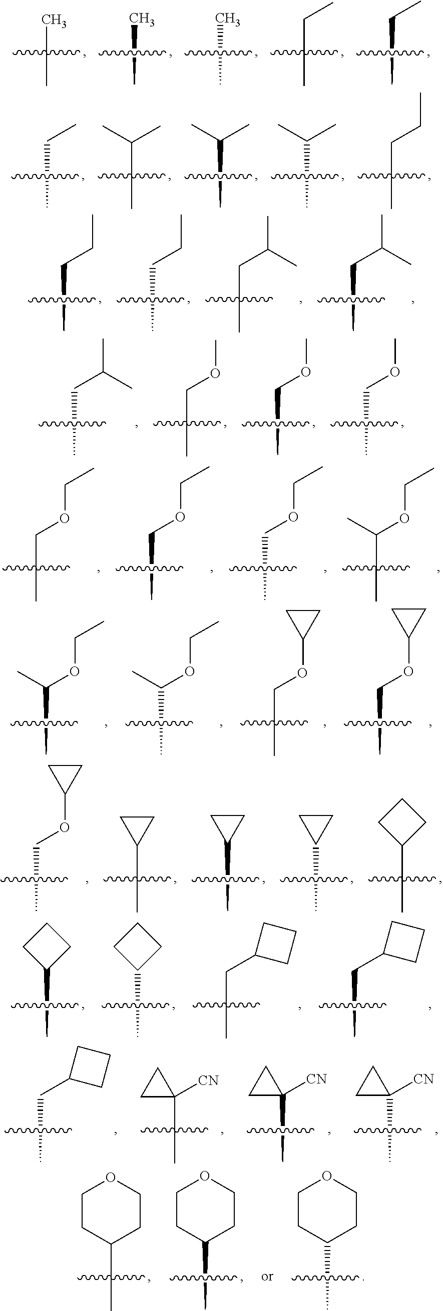

In some embodiments, R² is

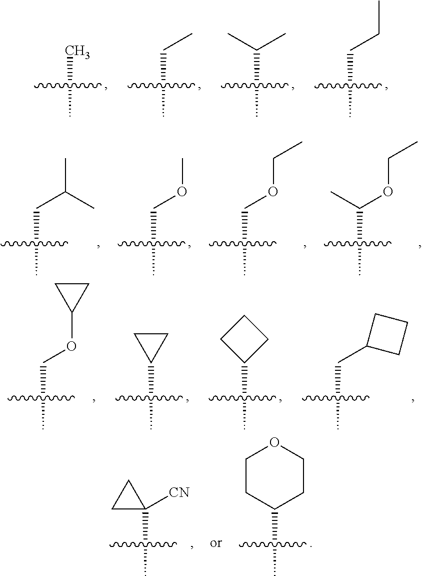

In some embodiments, R² is

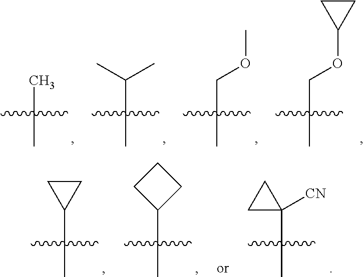

In some embodiments, R² is

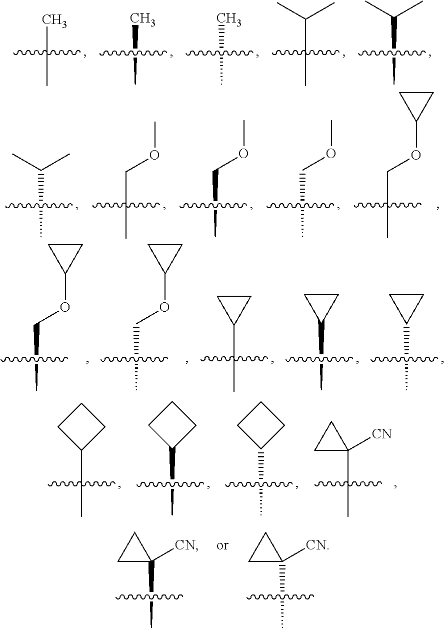

In some embodiments, R² is

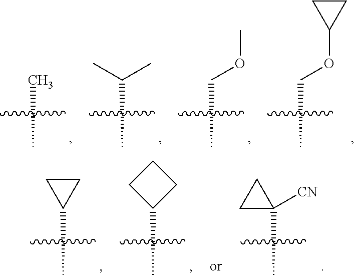

In some embodiments, R² is

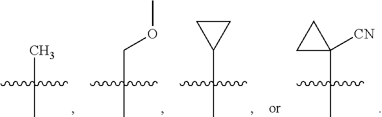

In some embodiments, R² is

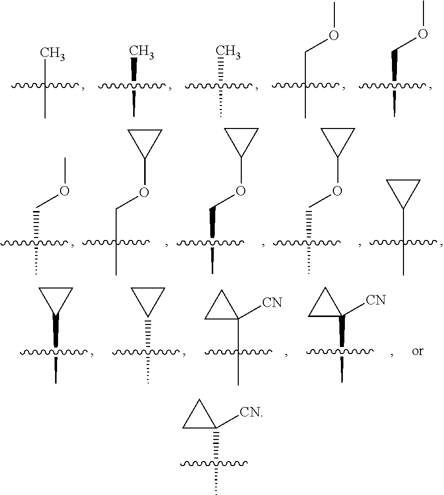

In some embodiments, R² is

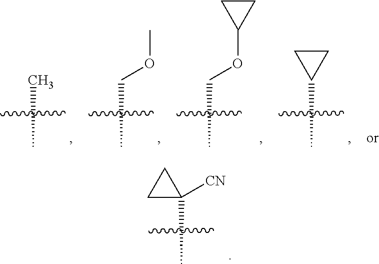

In some embodiments, R² is

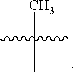

In some embodiments, R² is

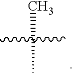

In some embodiments, R² is

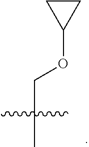 [SMILES: *COC1CC1]

In some embodiments, R² is

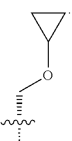

In some embodiments, R² is

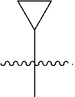 [SMILES: *C1CC1]

In some embodiments, R² is

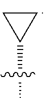

In some embodiments, R² is

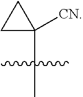 [SMILES: *C1(C#N)CC1]

In some embodiments, R² is

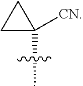

In some embodiments, disclosed herein is a compound of Formula (I), or a pharmaceutically acceptable salt thereof, wherein R³ is —C₍₃₋₆₎alkyl, —C₍₃₋₆₎cycloalkyl, —C₍₅₋₁₀₎polycycloalkyl, tetrahydropyranyl, —C₍₁₋₂₎alkyl-O—C₍₁₋₄₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, or —C₍₃₋₄₎cycloalkylC₍₁₋₃₎alkyl, wherein the —C₍₃₋₆₎alkyl, —C₍₃₋₆₎cycloalkyl, —C₍₅₋₁₀₎polycycloalkyl, tetrahydropyranyl, —C₍₁₋₂₎alkyl-O—C₍₁₋₄₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, and —C₍₃₋₄₎cycloalkylC₍₁₋₃₎alkyl are unsubstituted or substituted with one to three R^{3a} groups.

In some embodiments, disclosed herein is a compound of Formula (I), or a pharmaceutically acceptable salt thereof, wherein R³ is —C₍₃₋₆₎alkyl, —C₍₃₋₆₎cycloalkyl, —C₍₅₋₁₀₎polycycloalkyl, tetrahydropyranyl, —C₍₁₋₂₎alkyl-O—C₍₁₋₄₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, or —C₍₃₋₄₎cycloalkylC₍₁₋₃₎alkyl, wherein the —C₍₃₋₆₎alkyl, —C₍₃₋₆₎cycloalkyl, —C₍₅₋₁₀₎polycycloalkyl, tetrahydropyranyl, —C₍₁₋₂₎alkyl-O—C₍₁₋₄₎alkyl, —C₍₁₋₂₎alkylC₍₃₋₆₎cycloalkyl, C₍₁₋₂₎alkyl-O—C₍₃₋₆₎cycloalkyl, —C₍₁₋₂₎alkyl-C₍₅₋₁₀₎polycycloalkyl, and —C₍₃₋₄₎cycloalkylC₍₁₋₃₎alkyl are unsubstituted or substituted with one to three substituents selected from the group consisting of fluorine, —CH₃, —CH₂F, —CHF₂, and —CF₃. In some embodiments, R³ is —C₍₃₋₆₎alkyl, —C₍₃₋₆₎cycloalkyl, —C₍₅₋₈₎polycycloalkyl, or —C₍₁₋₂₎alkyl-O—C₍₁₋₄₎alkyl, each of which is unsubstituted or substituted with one to three fluorine atoms.

In some embodiments, R³ is

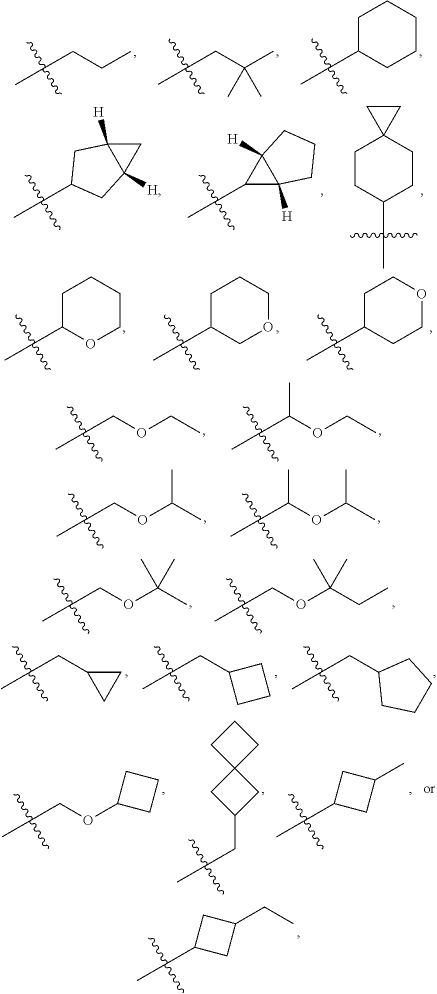

each of which is optionally substituted with one to three R^{3a} groups.

In some embodiments, R³ is

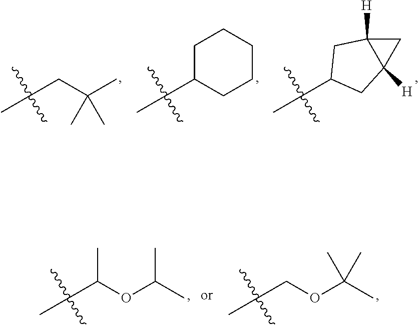

each of which is optionally substituted with one to three R^{3a} groups.

In some embodiments, R^{3a} independently for each occurrence is fluorine, —CH₃, —CH₂F, —CHF₂, or —CF₃.

In some embodiments, R^{3a} independently for each occurrence is fluorine, —CH₃, or —CF₃.

In some embodiments, R^{3a} independently for each occurrence is fluorine.

In some embodiments, R³ is

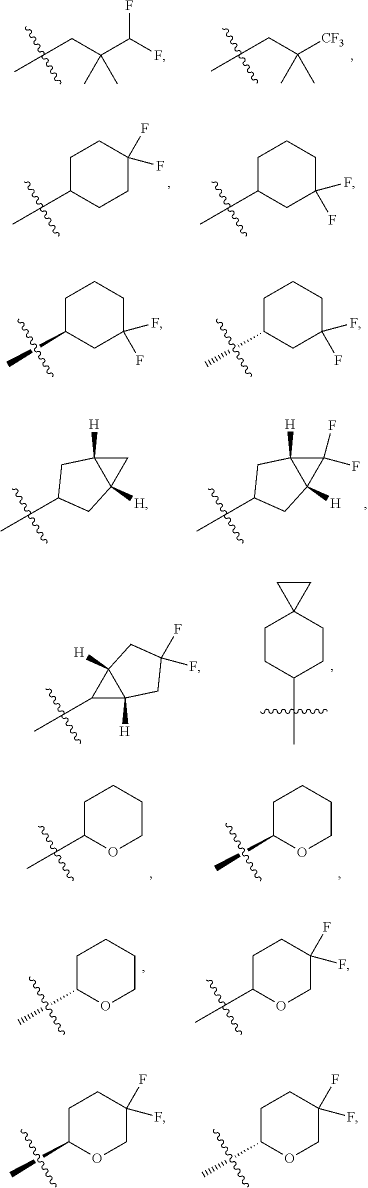

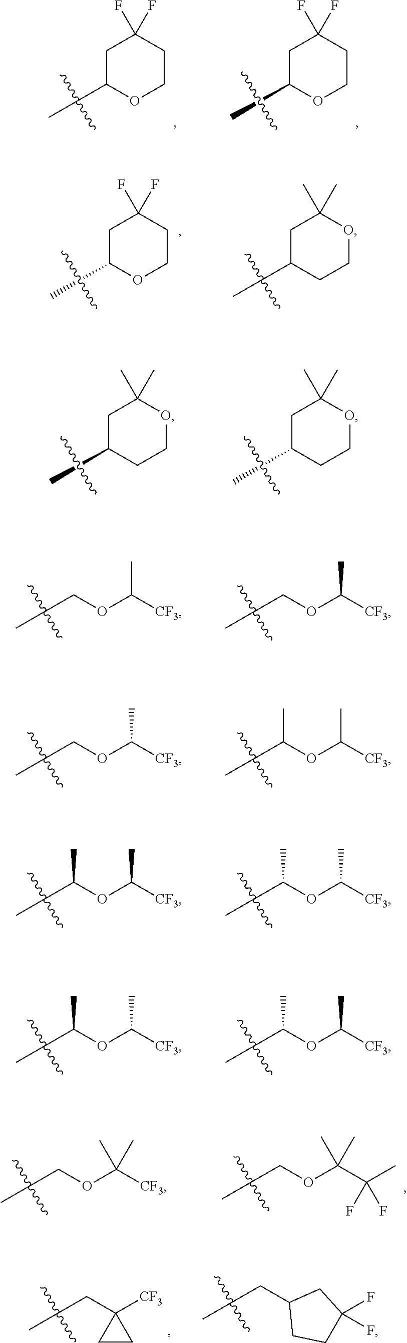

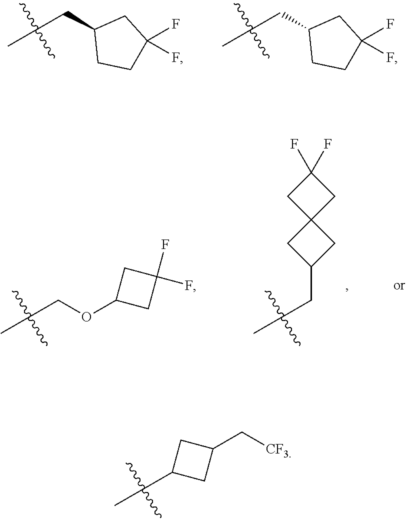 [SMILES: *C[C@@H]1CCC(F)(F)C1 ; *C[C@H]1CCC(F)(F)C1 ; *COC1CC(F)(F)C1 ; *CC1CC2(C1)CC(F)(F)C2 ; *C1CC(CC(F)(F)F)C1]

In some embodiments, R³ is

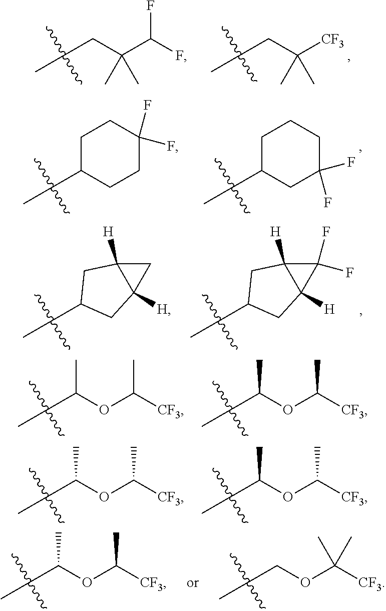

In some embodiments, R³ is

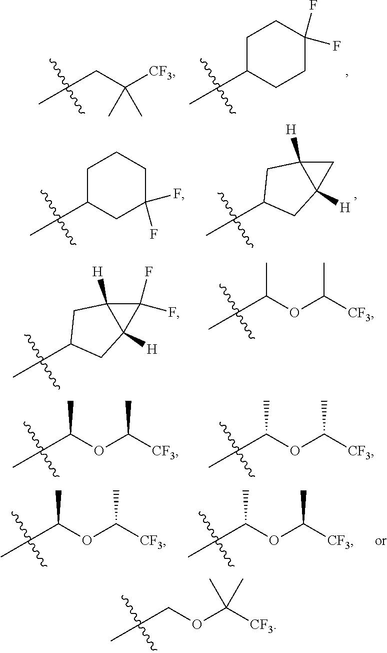

In some embodiments, R³ is

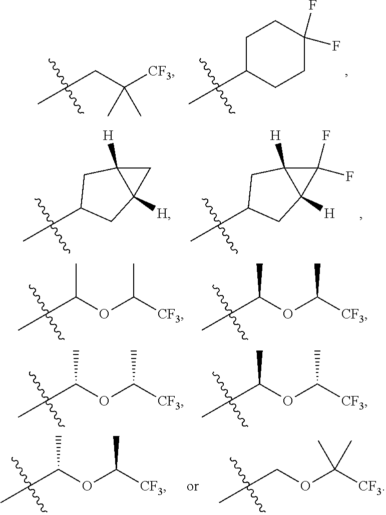

In some embodiments, R³ is

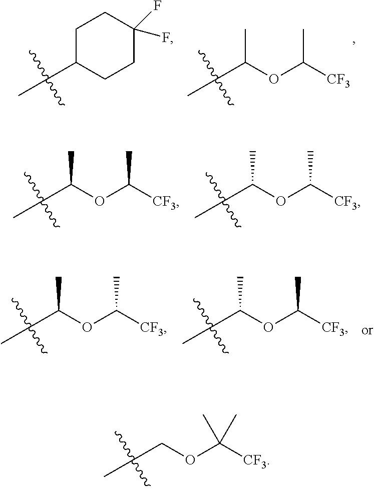

In some embodiments, R³ is

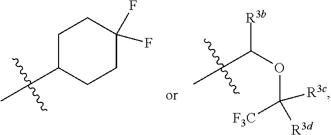

wherein R^{3b}, R^{3c}, and R^{3d} are each independently H or CH₃.

In some embodiments, R³ is

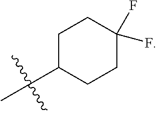 [SMILES: *C1CCC(F)(F)CC1]

In some embodiments, R³ is

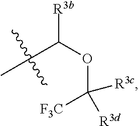

wherein R^{3b}, R^{3c}, and R^{3d} are each independently H or CH₃.

In some embodiments, R³ is

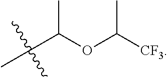 [SMILES: *C(C)OC(C)C(F)(F)F]

In some embodiments, R³ is

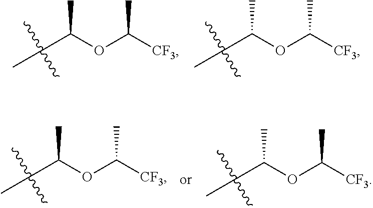

In some embodiments, R³ is

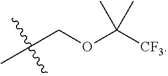 [SMILES: *COC(C)(C)C(F)(F)F]

In some embodiments, disclosed herein is a compound of Formula (I), or a pharmaceutically acceptable salt thereof, wherein R⁴ is —C₍₁₋₆₎)alkyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl, wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups, wherein the phenyl is unsubstituted or substituted with one to three R^{4b} groups, and wherein the 5-membered heteroaryl is unsubstituted or substituted with one to three R^{4c} groups.

In some embodiments, R⁴ is isopropyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl, wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups, wherein the phenyl is unsubstituted or substituted with one to three R^{4b} groups, and wherein the 5-membered heteroaryl is unsubstituted or substituted with one to three R^{4c} groups.

In some embodiments, R⁴ is isopropyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl, wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups, wherein the phenyl is unsubstituted or substituted with one to three R^{4b} groups, and wherein the 5-membered heteroaryl is substituted with one to three R^{4c} groups.

In some embodiments, R⁴ is —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl, wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups, wherein the phenyl is unsubstituted or substituted with one to three R^{4b} groups, and wherein the 5-membered heteroaryl is substituted with one to three R^{4c} groups.

In some embodiments, R⁴ is —C₍₃₋₆₎cycloalkyl, —C₍₅₋₈₎polycycloalkyl, —C₍₁₋₂₎alkyl-C₍₃₋₅₎cycloalkyl, each of which is unsubstituted or substituted with one to three R^{4a} groups, wherein each R^{4a} independently for each occurrence is fluorine, —C₍₁₋₃₎alkyl, or —CN, wherein the —C₍₁₋₃₎alkyl is unsubstituted or substituted with one to three fluorine atoms.

In some embodiments, R⁴ is —C₍₃₋₆₎cycloalkyl, —C₍₅₋₈₎polycycloalkyl, —C₍₁₋₂₎alkyl-C₍₃₋₅₎cycloalkyl, each of which is unsubstituted or substituted with one to three R^{4a} groups, wherein each R^{4a} a independently for each occurrence is fluorine, —CH₃, CH₂F, —CHF₂, —CF₃, or —CN.

In some embodiments, R⁴ is

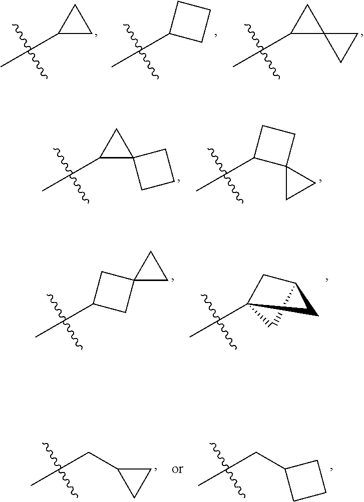

each of which is unsubstituted or substituted with one to three substituents selected from the group consisting of fluorine, —CH₃, CH₂F, —CHF₂, —CF₃, or —CN.

In some embodiments, R⁴ is

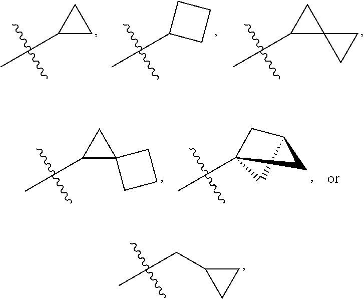

each of which is unsubstituted or substituted with one to three substituents selected from the group consisting of fluorine, —CH₃, CH₂F, —CHF₂, —CF₃, or —CN.

In some embodiments, R⁴ is cyclopropyl unsubstituted or substituted with one to three substituents selected from the group consisting of fluorine, —CH₃, CH₂F, —CHF₂, —CF₃, or —CN.

In some embodiments, R⁴ is:

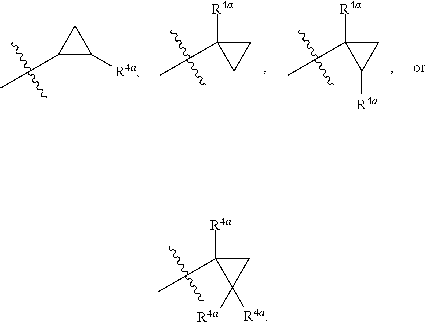

In some embodiments, R⁴ is:

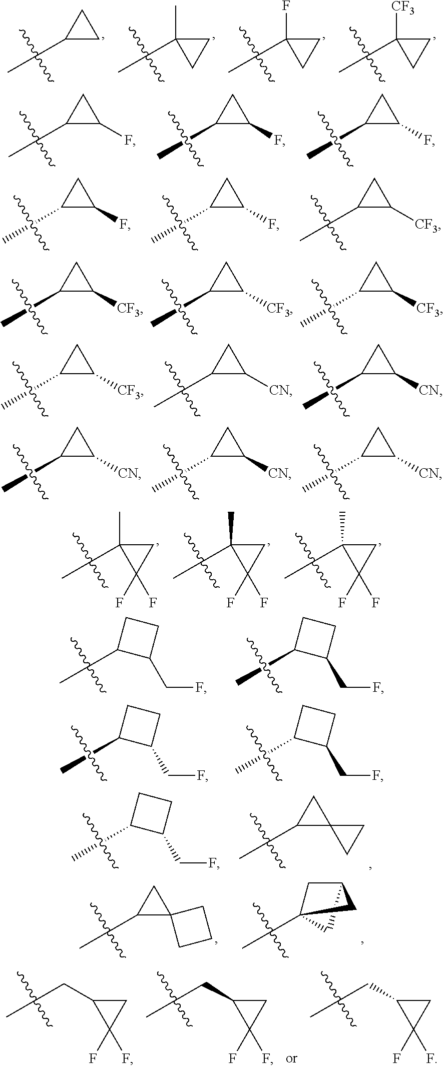

In some embodiments, R⁴ is phenyl, which is unsubstituted or substituted with one to three R^{4b} groups, wherein each R^{4b} independently for each occurrence is fluorine or —CN.

In some embodiments, R⁴ is:

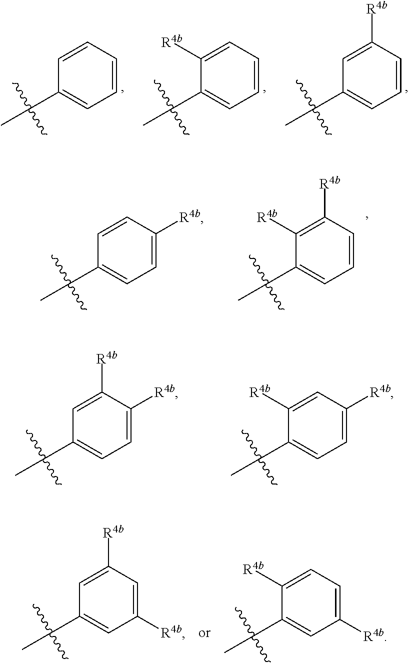

In some embodiments, R⁴ is:

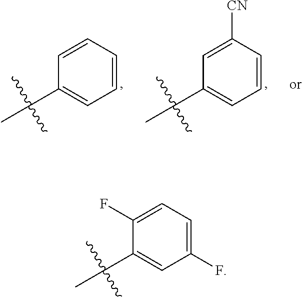 [SMILES: *c1ccccc1 ; *c1cccc(C#N)c1 ; *c1cc(F)ccc1F]

In some embodiments, R⁴ is:

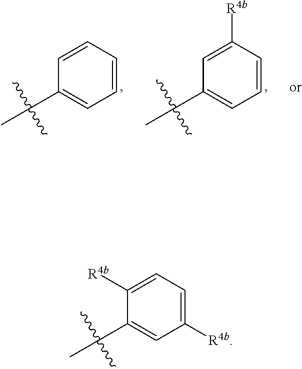

In some embodiments, R⁴ is 5-membered heteroaryl, which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl, are unsubstituted or substituted with one to six substituents independently selected from the group consisting of fluorine, —OH, and —CN; or, alternatively, two R^{4c} groups attached to vicinal atoms on the same ring can be combined with the atoms to which they are attached to form a C₍₃₋₆₎cycloalkyl or a 3- to 6-membered heterocyclyl.

In some embodiments, R⁴ is 5-membered heteroaryl, which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN; or, alternately, wherein two R^{4c} groups attached to vicinal atoms on the same ring are combined with the atoms to which they are attached to form a C₍₃₋₆₎cycloalkyl or a 3- to 6-membered heterocyclyl.

In some embodiments, R⁴ is 5-membered heteroaryl, which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN; or R⁴ is

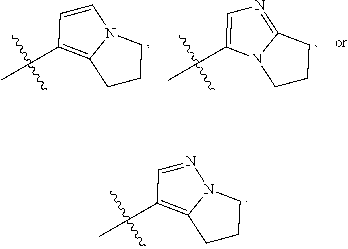 [SMILES: *c1ccn2c1CCC2 ; *c1cnc2n1CCC2 ; *c1cnn2c1CCC2]

In some embodiments, R⁴ is pyrrolyl, pyrazolyl, imidazolyl, 1,2,3-triazole, 1,2,4-triazole, pyrazolonyl, oxazolyl, isoxazolyl, 1,2,3-oxadiazaole, 1,2,4-oxadiazole, 1,2,5-oxadiazole, 1,3,4-oxadiazole, thiophenyl, thiazolyl, isothiazolyl, 1,2,3-thiadiazolyl, 1,2,4-thiadiazolyl, 1,2,5-thiadiazolyl, 1,3,4-thiadiazolyl, or pyridinyl, each of which is unsubstituted or substituted with one to three R^{4c} groups.

In some embodiments, R⁴ is pyrrolyl, pyrazolyl, imidazolyl, 1,2,3-triazole, 1,2,4-triazole, pyrazolonyl, oxazolyl, isoxazolyl, 1,2,3-oxadiazaole, 1,2,4-oxadiazole, 1,2,5-oxadiazole, 1,3,4-oxadiazole, thiophenyl, thiazolyl, isothiazolyl, 1,2,3-thiadiazolyl, 1,2,4-thiadiazolyl, 1,2,5-thiadiazolyl, 1,3,4-thiadiazolyl, or pyridinyl, each of which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl, are unsubstituted or substituted with one to six substituents independently selected from the group consisting of fluorine, —OH, and —CN; or, alternately, wherein two R^{4c} groups attached to vicinal atoms on the same ring are combined with the atoms to which they are attached to form a C₍₃₋₆₎cycloalkyl or a 3- to 6-membered heterocyclyl.

In some embodiments, R⁴ is pyrrolyl, pyrazolyl, imidazolyl, 1,2,3-triazole, 1,2,4-triazole, pyrazolonyl, oxazolyl, isoxazolyl, 1,2,3-oxadiazaole, 1,2,4-oxadiazole, 1,2,5-oxadiazole, 1,3,4-oxadiazole, thiophenyl, thiazolyl, isothiazolyl, 1,2,3-thiadiazolyl, 1,2,4-thiadiazolyl, 1,2,5-thiadiazolyl, 1,3,4-thiadiazolyl, or pyridinyl, each of which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN; or, alternately, wherein two R^{4c} groups attached to vicinal atoms on the same ring are combined with the atoms to which they are attached to form a C₍₃₋₆₎cycloalkyl or a 3- to 6-membered heterocyclyl.

In some embodiments, R⁴ is pyrrolyl, pyrazolyl, imidazolyl, 1,2,3-triazole, 1,2,4-triazole, pyrazolonyl, oxazolyl, isoxazolyl, 1,2,3-oxadiazaole, 1,2,4-oxadiazole, 1,2,5-oxadiazole, 1,3,4-oxadiazole, thiophenyl, thiazolyl, isothiazolyl, 1,2,3-thiadiazolyl, 1,2,4-thiadiazolyl, 1,2,5-thiadiazolyl, 1,3,4-thiadiazolyl, or pyridinyl, each of which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN; or R⁴ is

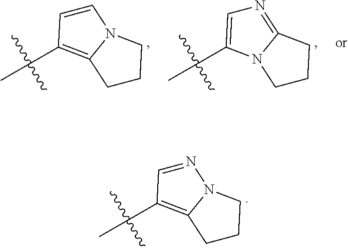 [SMILES: *c1ccn2c1CCC2 ; *c1cnc2n1CCC2 ; *c1cnn2c1CCC2]

In some embodiments, R⁴ is pyrrolyl, pyrazolyl, imidazolyl, 1,2,3-triazole, 1,2,4-triazole, pyrazolonyl, oxazolyl, isoxazolyl, 1,2,5-oxadiazole, 1,3,4-oxadiazole, thiophenyl, thiazolyl, 1,2,3-thiadiazolyl, or pyridinyl, each of which is unsubstituted or substituted with one to three R^{4c} groups

In some embodiments, R⁴ is pyrrolyl, pyrazolyl, imidazolyl, 1,2,3-triazole, 1,2,4-triazole, pyrazolonyl, oxazolyl, isoxazolyl, 1,2,5-oxadiazole, 1,3,4-oxadiazole, thiophenyl, thiazolyl, 1,2,3-thiadiazolyl, or pyridinyl, each of which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl, are unsubstituted or substituted with one to six substituents independently selected from the group consisting of fluorine, —OH, and —CN; or, alternately, wherein two R^{4c} groups attached to vicinal atoms on the same ring are combined with the atoms to which they are attached to form a C₍₃₋₆₎cycloalkyl or a 3- to 6-membered heterocyclyl.

In some embodiments, R⁴ is pyrrolyl, pyrazolyl, imidazolyl, 1,2,3-triazole, 1,2,4-triazole, pyrazolonyl, oxazolyl, isoxazolyl, 1,2,5-oxadiazole, 1,3,4-oxadiazole, thiophenyl, thiazolyl, 1,2,3-thiadiazolyl, or pyridinyl, each of which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN; or, alternately, wherein two R^{4c} groups attached to vicinal atoms on the same ring are combined with the atoms to which they are attached to form a C₍₃₋₆₎cycloalkyl or a 3- to 6-membered heterocyclyl.

In some embodiments, R⁴ is pyrrolyl, pyrazolyl, imidazolyl, 1,2,3-triazole, 1,2,4-triazole, pyrazolonyl, oxazolyl, isoxazolyl, 1,2,5-oxadiazole, 1,3,4-oxadiazole, thiophenyl, thiazolyl, 1,2,3-thiadiazolyl, or pyridinyl, each of which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN; or R⁴ is:

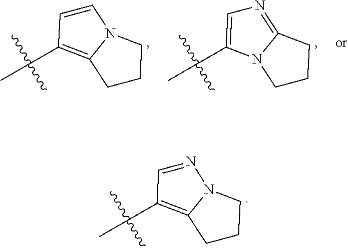 [SMILES: *c1ccn2c1CCC2 ; *c1cnc2n1CCC2 ; *c1cnn2c1CCC2]

In some embodiments, R⁴ is pyrrolyl, pyrazolyl, 1,2,3-triazole, isoxazolyl, 1,2,5-oxadiazole, thiophenyl, thiazolyl, or 1,2,3-thiadiazolyl, each of which is unsubstituted or substituted with one to three R^{4c} groups.

In some embodiments, R⁴ is pyrrolyl, pyrazolyl, 1,2,3-triazole, isoxazolyl, 1,2,5-oxadiazole, thiophenyl, thiazolyl, or 1,2,3-thiadiazolyl, each of which is unsubstituted or substituted with one to three R^{4c} groups, wherein each R^{4c} independently for each occurrence is fluorine, —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl, are unsubstituted or substituted with one to six substituents independently selected from the group consisting of fluorine, —OH, and —CN; or, alternately, wherein two R^{4c} groups attached to vicinal atoms on the same ring are combined with the atoms to which they are attached to form a C₍₃₋₆₎cycloalkyl or a 3- to 6-membered heterocyclyl.

In some embodiments, R⁴ is pyrrolyl, pyrazolyl, 1,2,3-triazole, isoxazolyl, 1,2,5-oxadiazole, thiophenyl, thiazolyl, or 1,2,3-thiadiazolyl, each of which is unsubstituted or substituted with one to three R^{4c} groups, wherein each R^{4c} independently for each occurrence is fluorine, —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN; or, alternately, wherein two R^{4c} groups attached to vicinal atoms on the same ring are combined with the atoms to which they are attached to form a C₍₃₋₆₎cycloalkyl or a 3- to 6-membered heterocyclyl.

In some embodiments, R⁴ is pyrrolyl, pyrazolyl, 1,2,3-triazole, isoxazolyl, 1,2,5-oxadiazole, thiophenyl, thiazolyl, or 1,2,3-thiadiazolyl, each of which is unsubstituted or substituted with one to three R^{4c} groups, wherein each R^{4c} independently for each occurrence is fluorine, —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN; or R⁴ is:

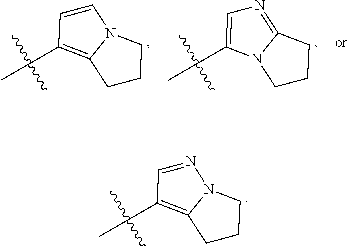 [SMILES: *c1ccn2c1CCC2 ; *c1cnc2n1CCC2 ; *c1cnn2c1CCC2]

In some embodiments, R⁴ is pyrrolyl, pyrazolyl, 1,2,3-triazole, isoxazolyl, 1,2,5-oxadiazole, thiophenyl, thiazolyl, or 1,2,3-thiadiazolyl, each of which is unsubstituted or substituted with one to three R^{4c} groups, wherein each R^{4c} independently for each occurrence is fluorine, —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN.

In some embodiments, R⁴ is pyrazolyl, isoxazolyl, or 1,2,5-oxadiazole, each of which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl, are unsubstituted or substituted with one to six substituents independently selected from the group consisting of fluorine, —OH, and —CN.

In some embodiments, R⁴ is pyrazolyl, isoxazolyl, or 1,2,5-oxadiazole, each of which is unsubstituted or substituted with one to three R^{4c} groups, wherein each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN.

In some embodiments, R⁴ is pyrazolyl, isoxazolyl, or 1,2,5-oxadiazole, each of which is unsubstituted or substituted with one R^{4c} groups, wherein R^{4c} is —C₍₁₋₆₎)alkyl, —C₍₃₋₆₎cycloalkyl, or —O—C₍₁₋₃₎alkyl, wherein the —C₍₁₋₆₎alkyl, —C₍₃₋₆₎cycloalkyl, and —O—C₍₁₋₃₎alkyl are unsubstituted or substituted with one to three fluorines.

In some embodiments, R⁴ is 5-membered heteroaryl, which is substituted with one substituent selected from the group consisting of —C₍₁₋₃₎alkyl, —C₍₃₋₄₎cycloalkyl, or —O—C₍₁₋₃₎alkyl, each of which is unsubstituted or further substituted with one to three fluorines.

In some embodiments, R⁴ is:

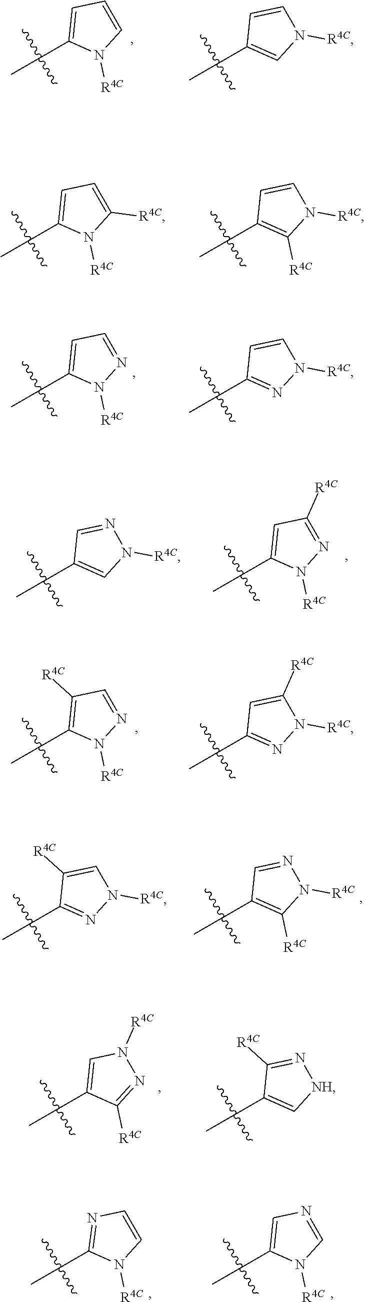

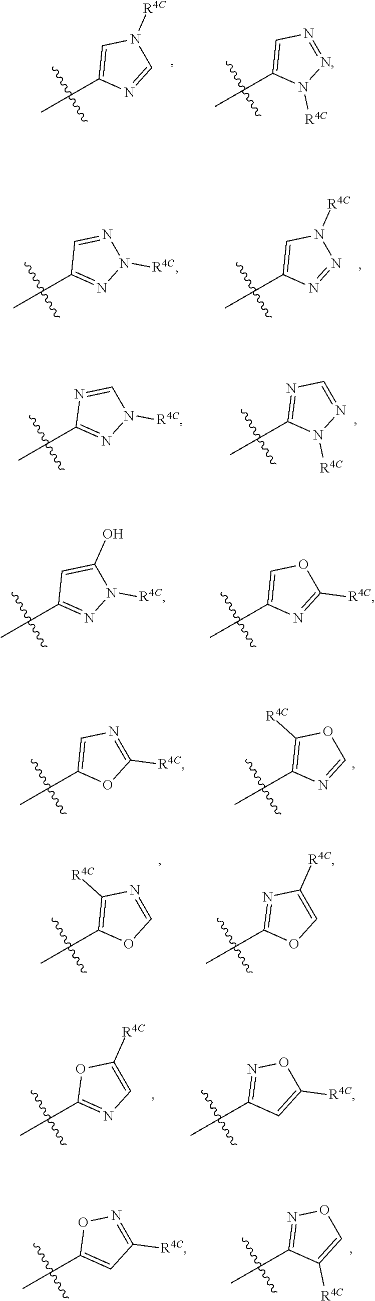

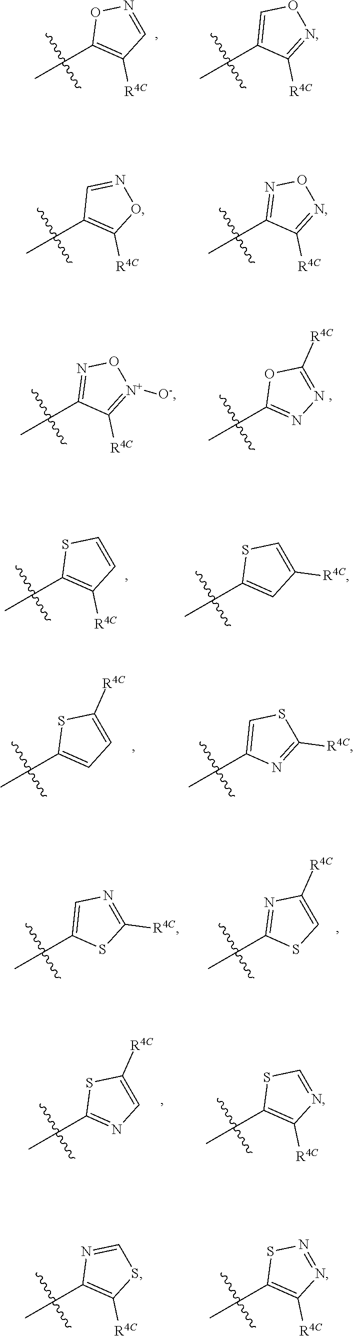

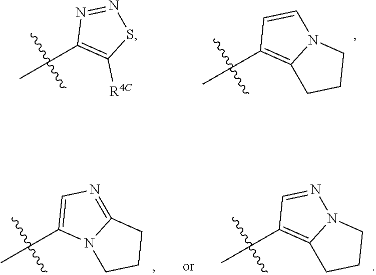

In some embodiments, R⁴ is:

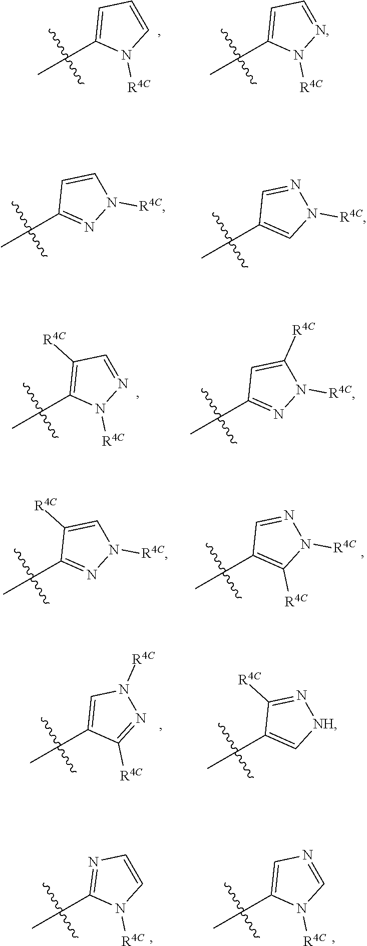

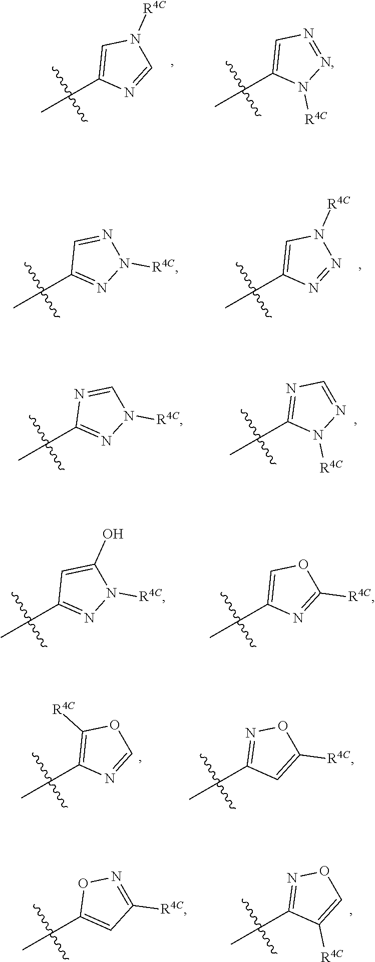

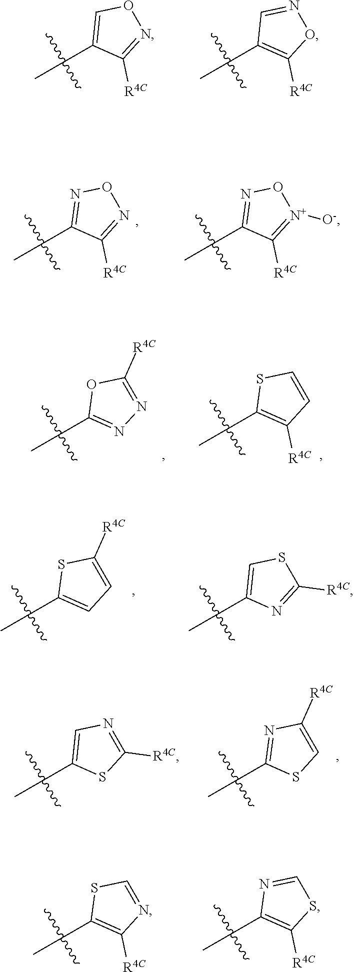

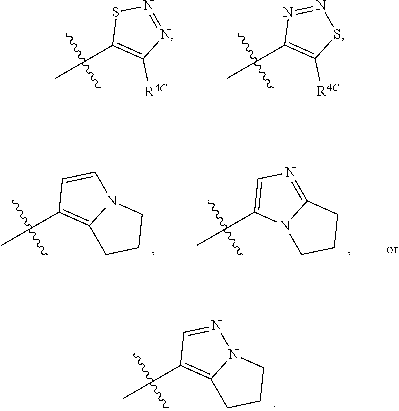

In some embodiments, R⁴ is:

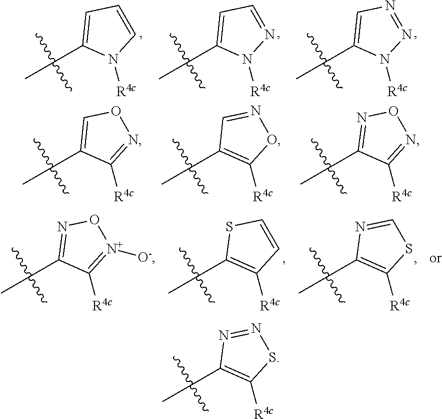

In some embodiments, R⁴ is

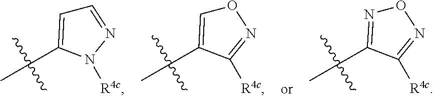

In some embodiments, each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN.

In some embodiments, each R^{4c} independently for each occurrence is fluorine, —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, and —O—C₍₁₋₃₎alkyl are unsubstituted or substituted with one to six substituents independently selected from the group consisting of fluorine, —OH, and —CN.

In some embodiments, each R^{4c} independently for each occurrence is:

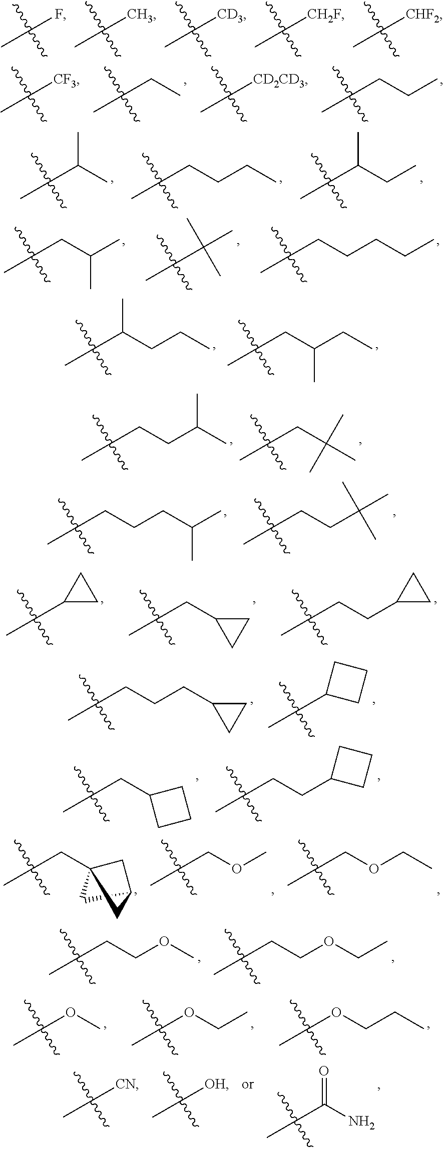

wherein 1, 2, 3, or 4 hydrogen atoms not explicitly denoted “H” are optionally replaced with fluorine, —OH, or —CN.

In some embodiments, each R^{4c} independently for each occurrence is:

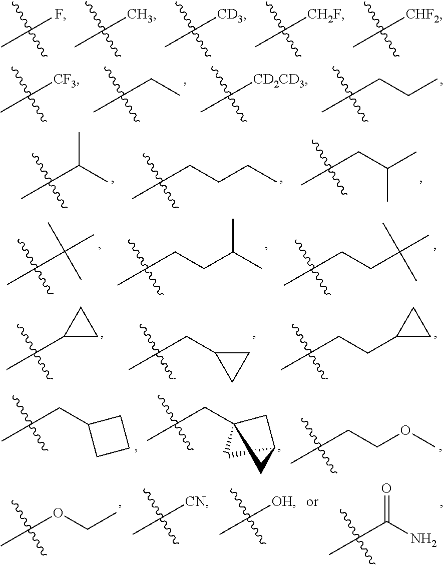

wherein 1, 2, 3, or 4 hydrogen atoms not explicitly denoted “H” are optionally replaced with fluorine, —OH, or —CN.

In some embodiments, each R^{4c} independently for each occurrence is:

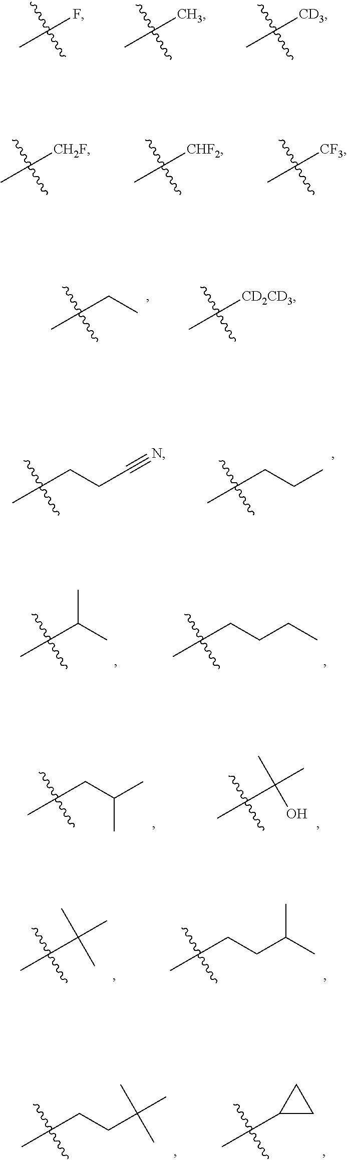

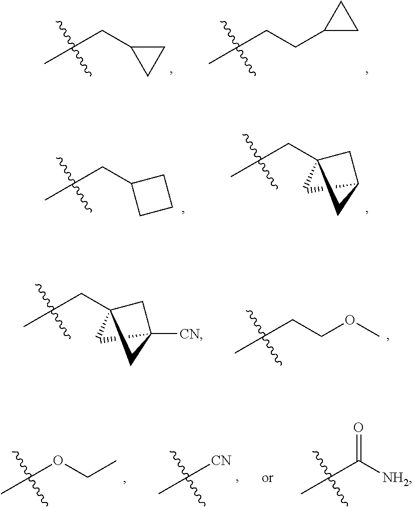

wherein 1, 2, 3, or 4 hydrogen atoms not explicitly denoted “H” are optionally replaced with fluorine.

In some embodiments, each R^{4c} independently for each occurrence is:

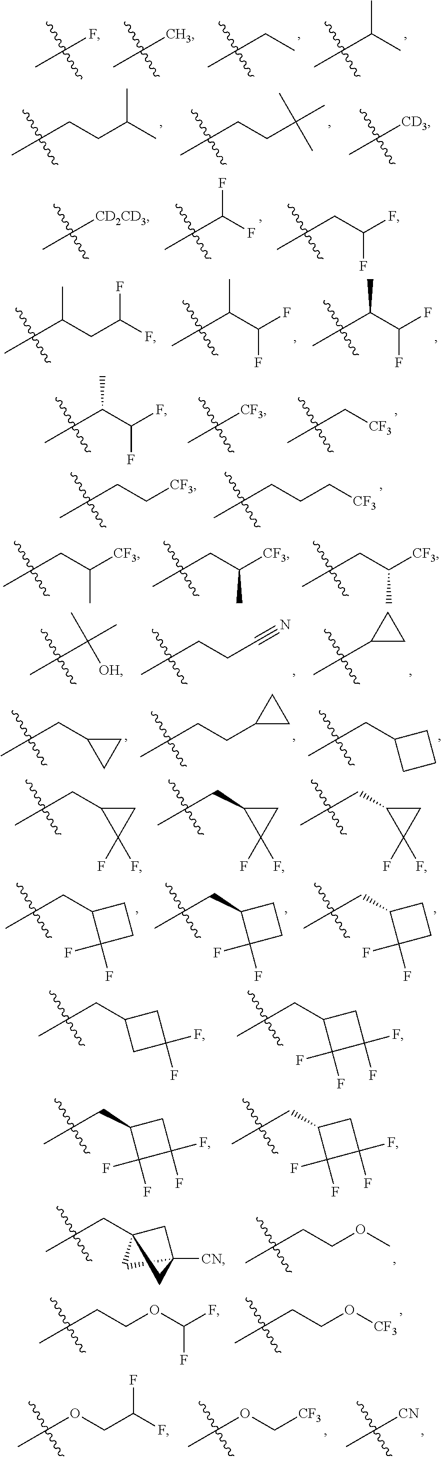

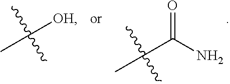

In some embodiments, R^{4c} is:

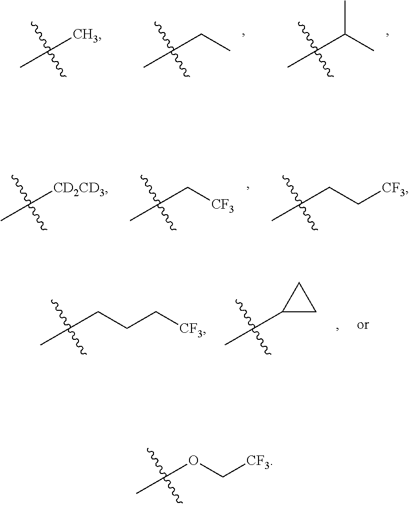

In some embodiments, R^{4c} is:

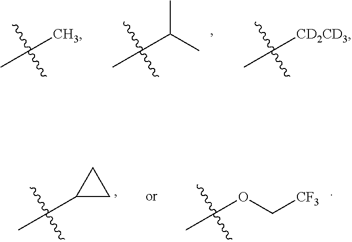

In some embodiments, R⁴ is:

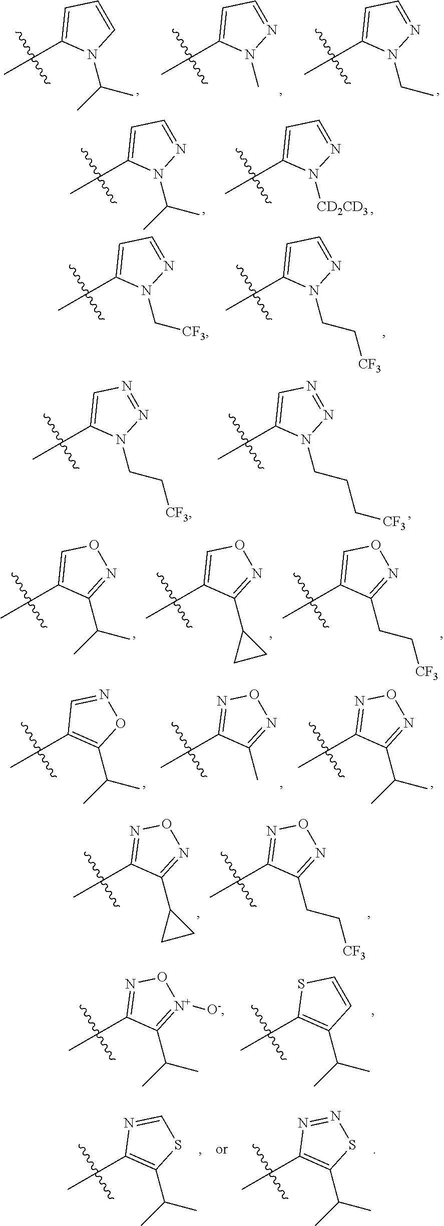

In some embodiments, R⁴ is:

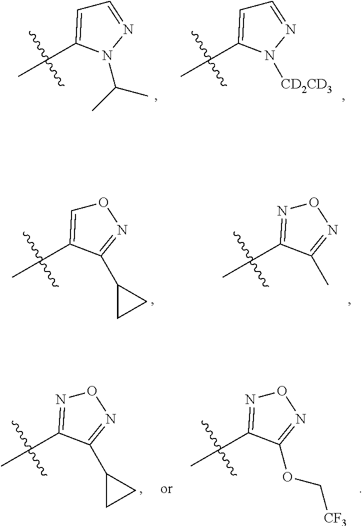

In some embodiments, R⁴ is:

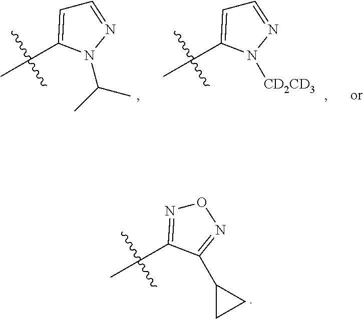

In some embodiments, R⁴ is:

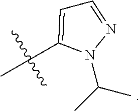 [SMILES: *c1ccnn1C(C)C]

In some embodiments, R⁴ is:

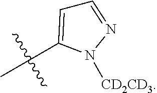

In some embodiments, R⁴ is:

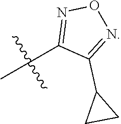 [SMILES: *c1nonc1C1CC1]

In some embodiments, disclosed herein is a compound of Formula (I), or a pharmaceutically acceptable salt thereof, wherein:
R¹ is —C₍₁₋₄₎alkyl, —CH₂—C₍₃₋₄₎cycloalkyl, or —CH₂—C₍₅₋₈₎polycycloalkyl, each of which is unsubstituted or substituted with one to six fluorine atoms;
R² is —C₍₁₋₃₎alkyl, cyclopropyl, cyclobutyl, or C₍₁₋₂₎alkyl-O—C₍₁₋₂₎alkyl, wherein the cyclopropyl is unsubstituted or substituted with one —CN;
R³ is —C₍₃₋₆₎alkyl, —C₍₃₋₆₎cycloalkyl, —C₍₅₋₈₎polycycloalkyl, or —C₍₁₋₂₎alkyl-O—C(1-s)alkyl, each of which is unsubstituted or substituted with one to three fluorine atoms; and
R⁴ is 5-membered heteroaryl, which is substituted with one substituent selected from the group consisting of —C₍₁₋₃₎alkyl, —C₍₃₋₄₎cycloalkyl, or —O—C₍₁₋₃₎alkyl, each of which is unsubstituted or further substituted with one to three fluorines.

In some embodiments, disclosed herein is a compound of Formula (I), or a pharmaceutically acceptable salt thereof, wherein:
R¹ is:

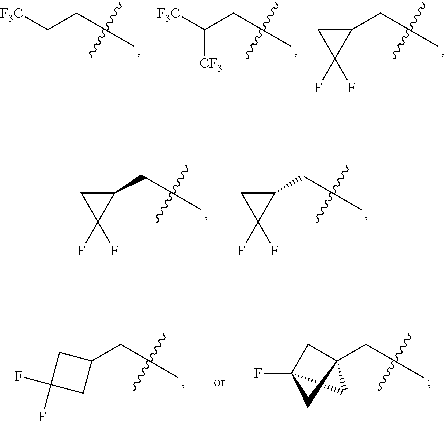

R² is:

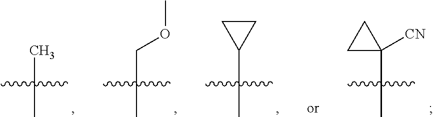

R³ is:

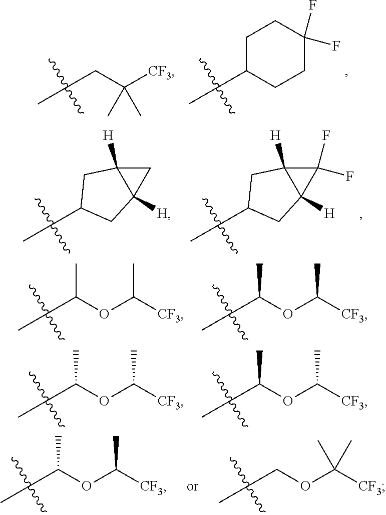

R⁴ is:

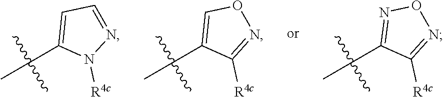

R^{4c} is:

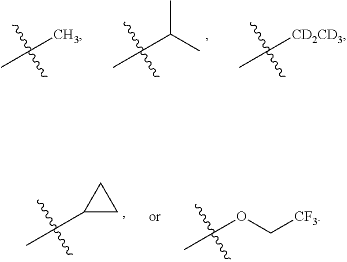

In some embodiments, disclosed herein is a compound of Formula (I), or a pharmaceutically acceptable salt thereof, which is a compound of formula Ib:

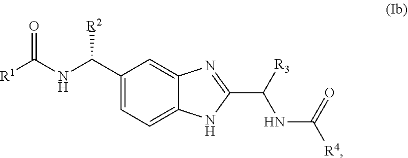

wherein R¹, R², R³, and R⁴ are as defined herein.

In some embodiments, disclosed herein is a compound of Formula (I), or a pharmaceutically acceptable salt thereof, which is a compound of formula Ib-1a:

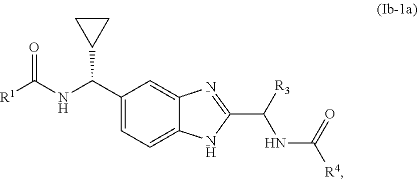

wherein R¹, R³, and R⁴ are as defined herein.

In some embodiments, disclosed herein is a compound of Formula (I), or a pharmaceutically acceptable salt thereof, which is a compound of formula Ib-1b:

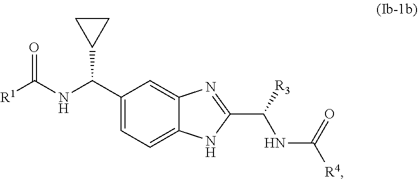

wherein R¹, R³, and R⁴ are as defined herein.

In some embodiments, disclosed herein is a compound of Formula (I), or a pharmaceutically acceptable salt thereof, which is a compound of formula Ib-2a:

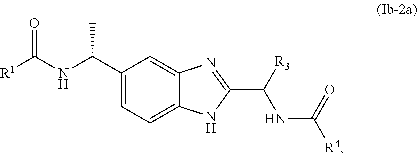

wherein R¹, R³, and R⁴ are as defined herein.

In some embodiments, disclosed herein is a compound of Formula (I), or a pharmaceutically acceptable salt thereof, which is a compound of formula Ib-2b:

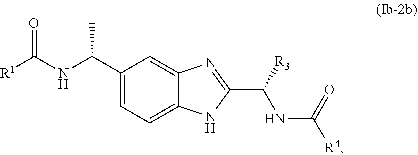

wherein R¹, R³, and R⁴ are as defined herein.

In some embodiments, disclosed herein is a compound of Formula (I), or a pharmaceutically acceptable salt thereof, which is a compound of formula Ib-3a:

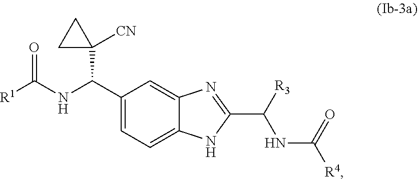

wherein R¹, R³, and R⁴ are as defined herein.

In some embodiments, disclosed herein is a compound of Formula (I), or a pharmaceutically acceptable salt thereof, which is a compound of formula Ib-3b:

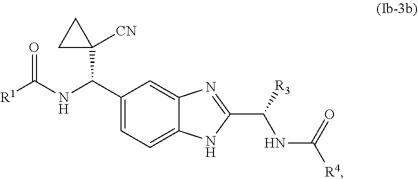

wherein R¹, R³, and R⁴ are as defined herein.

In some embodiments, disclosed herein is a compound of Formula (I), or a pharmaceutically acceptable salt thereof, which is a compound of formula Ic:

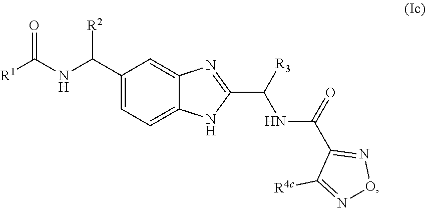

wherein R¹, R², R³, and R^{4c} are as defined herein.

In some embodiments,
R¹ is —C₍₁₋₃₎alkyl-C₍₃₋₆₎cycloalkyl, which is unsubstituted or substituted with one, two, or three fluorines;
R² is —C₍₁₋₃₎alkyl, cyclopropyl, cyclobutyl, or C₍₁₋₂₎alkyl-O—C₍₁₋₂₎alkyl, wherein the cyclopropyl is unsubstituted or substituted with one —CN;
R³ is —C₍₁₋₂₎alkyl-O—C₍₁₋₅₎alkyl, which is unsubstituted or substituted with one, two, or three substituents selected from the group consisting of fluorine, —CH₃, —CH₂F, —CHF₂, and —CF₃; and
R^{4c} is —C₍₁₋₃₎alkyl or —C₍₃₋₄₎cycloalkyl.

In some embodiments, disclosed herein is a compound of Formula (I), or a pharmaceutically acceptable salt thereof, which is a compound of formula Ic-1a:

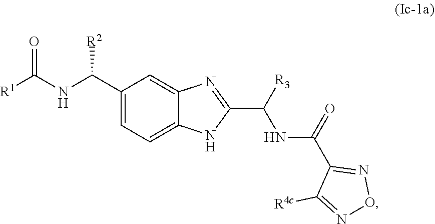

wherein R¹, R², R³, and R^{4c} are as defined herein.

In some embodiments,
R¹ is —C₍₁₋₃₎alkyl-C₍₃₋₆₎cycloalkyl, which is unsubstituted or substituted with one, two, or three fluorines;
R² is —C₍₁₋₃₎alkyl, cyclopropyl, cyclobutyl, or C₍₁₋₂₎alkyl-O—C₍₁₋₂₎alkyl, wherein the cyclopropyl is unsubstituted or substituted with one —CN;
R³ is —C₍₁₋₂₎alkyl-O—C₍₁₋₅₎alkyl, which is unsubstituted or substituted with one, two, or three substituents selected from the group consisting of fluorine, —CH₃, —CH₂F, —CHF₂, and —CF₃; and
R^{4c} is —C₍₁₋₃₎alkyl or —C₍₃₋₄₎cycloalkyl.

In some embodiments, disclosed herein is a compound of Formula (I), or a pharmaceutically acceptable salt thereof, which is a compound of formula Ic-1b:

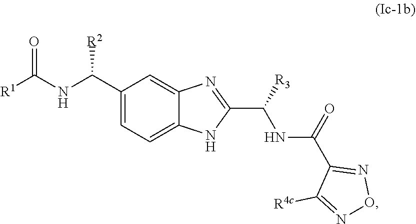

wherein R¹, R², R³, and R^{4c} are as defined herein.

In some embodiments,
R¹ is —C₍₁₋₃₎alkyl-C₍₃₋₆₎cycloalkyl, which is unsubstituted or substituted with one, two, or three fluorines;
R² is —C₍₁₋₃₎alkyl, cyclopropyl, cyclobutyl, or C₍₁₋₂₎alkyl-O—C₍₁₋₂₎alkyl, wherein the cyclopropyl is unsubstituted or substituted with one —CN;
R³ is —C₍₁₋₂₎alkyl-O—C₍₁₋₅₎alkyl, which is unsubstituted or substituted with one, two, or three substituents selected from the group consisting of fluorine, —CH₃, —CH₂F, —CHF₂, and —CF₃; and
R^{4c} is —C₍₁₋₃₎alkyl or —C₍₃₋₄₎cycloalkyl.

In some embodiments, disclosed herein is a compound of Formula (I), or a pharmaceutically acceptable salt thereof, having a structure as shown in any one of Tables 1A, 1B, 1C, 1D, 1E, 1F, 1G, 1H, 1I, 1J, 1K and IL.

TABLE 1A

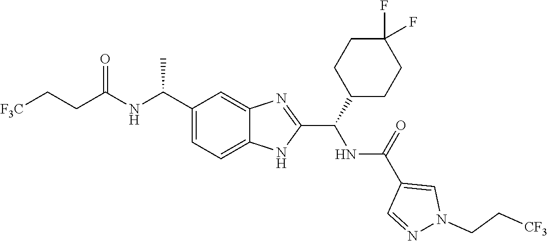 [SMILES: C[C@@H](NC(=O)CCC(F)(F)F)c1ccc2[nH]c([C@@H](NC(=O)c3cnn(CCC(F)(F)F)c3)C3CCC(F)(F)CC3)nc2c1]

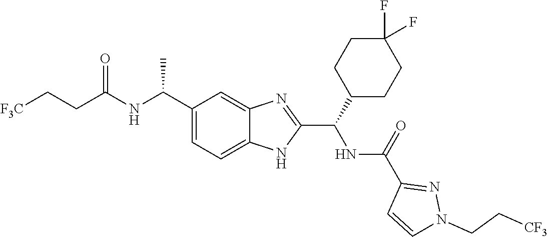 [SMILES: C[C@@H](NC(=O)CCC(F)(F)F)c1ccc2[nH]c([C@@H](NC(=O)c3ccn(CCC(F)(F)F)n3)C3CCC(F)(F)CC3)nc2c1]

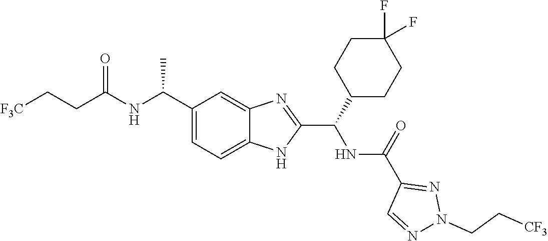 [SMILES: C[C@@H](NC(=O)CCC(F)(F)F)c1ccc2[nH]c([C@@H](NC(=O)c3cnn(CCC(F)(F)F)n3)C3CCC(F)(F)CC3)nc2c1]

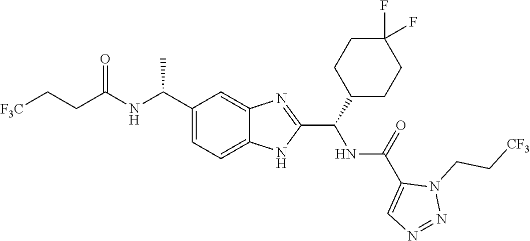 [SMILES: C[C@@H](NC(=O)CCC(F)(F)F)c1ccc2[nH]c([C@@H](NC(=O)c3cnnn3CCC(F)(F)F)C3CCC(F)(F)CC3)nc2c1]

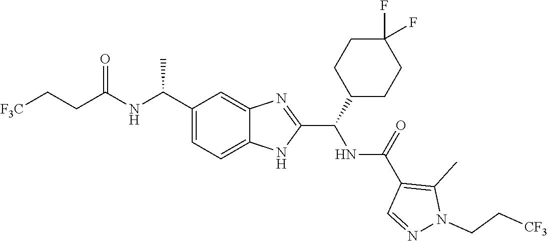 [SMILES: Cc1c(C(=O)N[C@H](c2nc3cc([C@@H](C)NC(=O)CCC(F)(F)F)ccc3[nH]2)C2CCC(F)(F)CC2)cnn1CCC(F)(F)F]

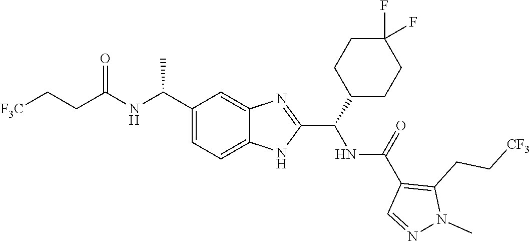 [SMILES: C[C@@H](NC(=O)CCC(F)(F)F)c1ccc2[nH]c([C@@H](NC(=O)c3cnn(C)c3CCC(F)(F)F)C3CCC(F)(F)CC3)nc2c1]

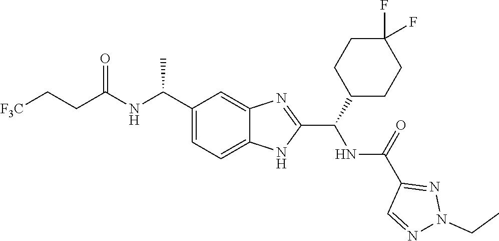 [SMILES: CCn1ncc(C(=O)N[C@H](c2nc3cc([C@@H](C)NC(=O)CCC(F)(F)F)ccc3[nH]2)C2CCC(F)(F)CC2)n1]

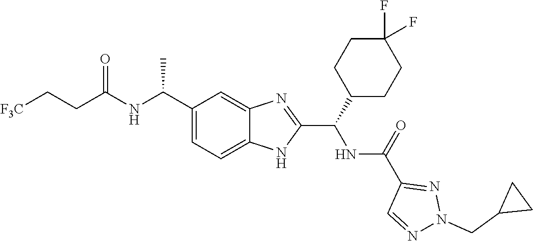 [SMILES: C[C@@H](NC(=O)CCC(F)(F)F)c1ccc2[nH]c([C@@H](NC(=O)c3cnn(CC4CC4)n3)C3CCC(F)(F)CC3)nc2c1]

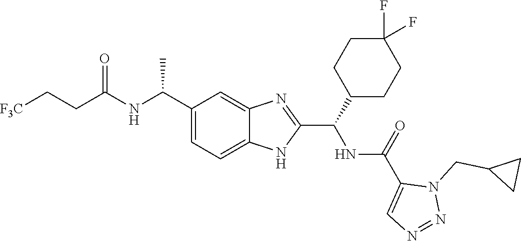 [SMILES: C[C@@H](NC(=O)CCC(F)(F)F)c1ccc2[nH]c([C@@H](NC(=O)c3cnnn3CC3CC3)C3CCC(F)(F)CC3)nc2c1]

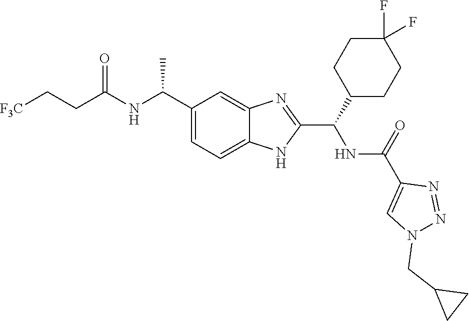 [SMILES: C[C@@H](NC(=O)CCC(F)(F)F)c1ccc2[nH]c([C@@H](NC(=O)c3cn(CC4CC4)nn3)C3CCC(F)(F)CC3)nc2c1]

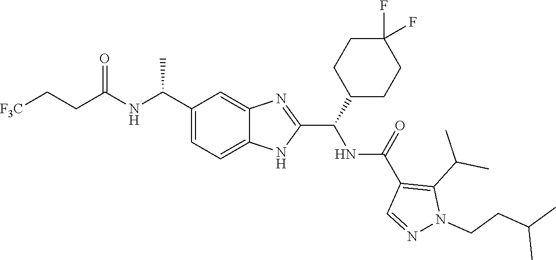 [SMILES: CC(C)CCn1ncc(C(=O)N[C@H](c2nc3cc([C@@H](C)NC(=O)CCC(F)(F)F)ccc3[nH]2)C2CCC(F)(F)CC2)c1C(C)C]

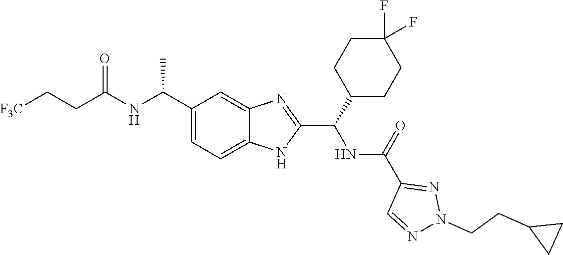 [SMILES: C[C@@H](NC(=O)CCC(F)(F)F)c1ccc2[nH]c([C@@H](NC(=O)c3cnn(CCC4CC4)n3)C3CCC(F)(F)CC3)nc2c1]

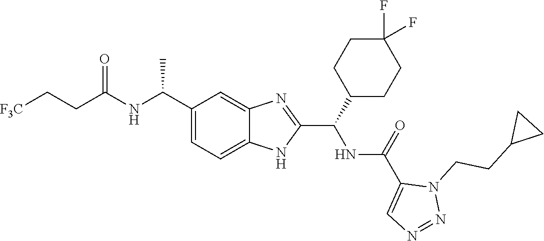 [SMILES: C[C@@H](NC(=O)CCC(F)(F)F)c1ccc2[nH]c([C@@H](NC(=O)c3cnnn3CCC3CC3)C3CCC(F)(F)CC3)nc2c1]

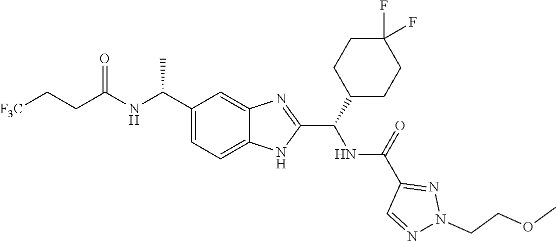 [SMILES: COCCn1ncc(C(=O)N[C@H](c2nc3cc([C@@H](C)NC(=O)CCC(F)(F)F)ccc3[nH]2)C2CCC(F)(F)CC2)n1]

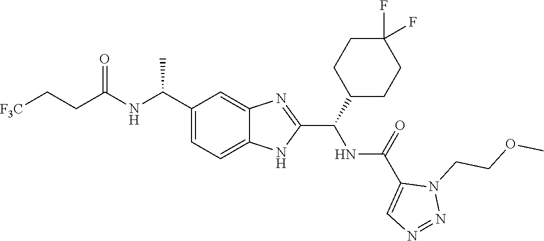 [SMILES: COCCn1nncc1C(=O)N[C@H](c1nc2cc([C@@H](C)NC(=O)CCC(F)(F)F)ccc2[nH]1)C1CCC(F)(F)CC1]

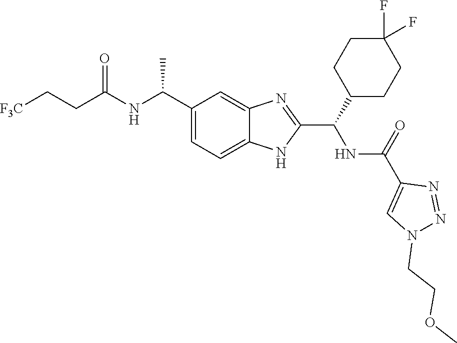 [SMILES: COCCn1cc(C(=O)N[C@H](c2nc3cc([C@@H](C)NC(=O)CCC(F)(F)F)ccc3[nH]2)C2CCC(F)(F)CC2)nn1]

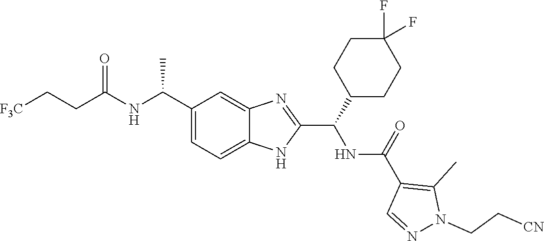 [SMILES: Cc1c(C(=O)N[C@H](c2nc3cc([C@@H](C)NC(=O)CCC(F)(F)F)ccc3[nH]2)C2CCC(F)(F)CC2)cnn1CCC#N]

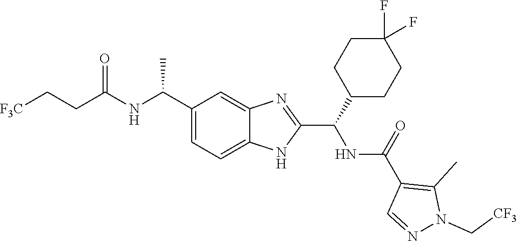 [SMILES: Cc1c(C(=O)N[C@H](c2nc3cc([C@@H](C)NC(=O)CCC(F)(F)F)ccc3[nH]2)C2CCC(F)(F)CC2)cnn1CC(F)(F)F]

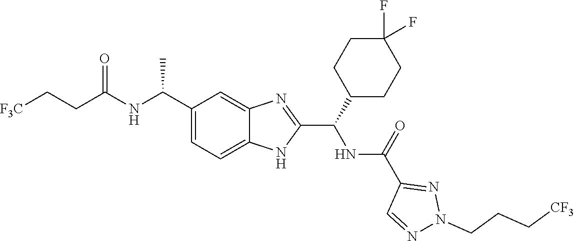 [SMILES: C[C@@H](NC(=O)CCC(F)(F)F)c1ccc2[nH]c([C@@H](NC(=O)c3cnn(CCCC(F)(F)F)n3)C3CCC(F)(F)CC3)nc2c1]

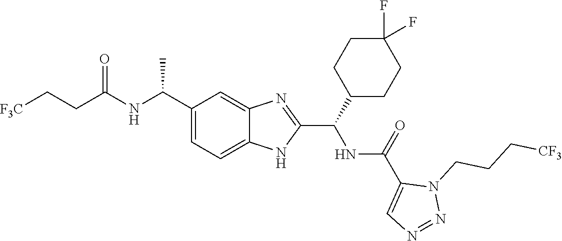 [SMILES: C[C@@H](NC(=O)CCC(F)(F)F)c1ccc2[nH]c([C@@H](NC(=O)c3cnnn3CCCC(F)(F)F)C3CCC(F)(F)CC3)nc2c1]

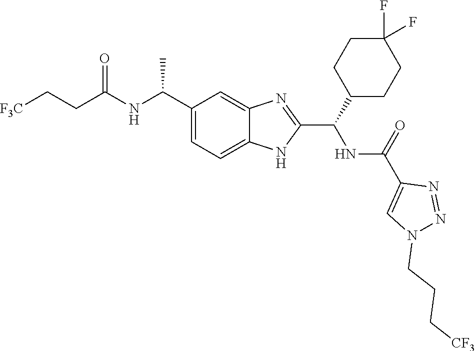 [SMILES: C[C@@H](NC(=O)CCC(F)(F)F)c1ccc2[nH]c([C@@H](NC(=O)c3cn(CCCC(F)(F)F)nn3)C3CCC(F)(F)CC3)nc2c1]

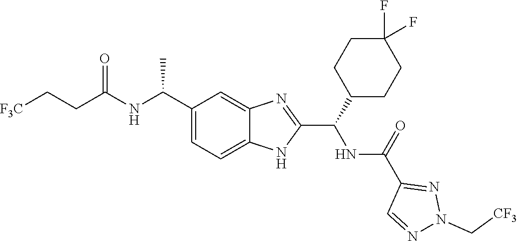 [SMILES: C[C@@H](NC(=O)CCC(F)(F)F)c1ccc2[nH]c([C@@H](NC(=O)c3cnn(CC(F)(F)F)n3)C3CCC(F)(F)CC3)nc2c1]

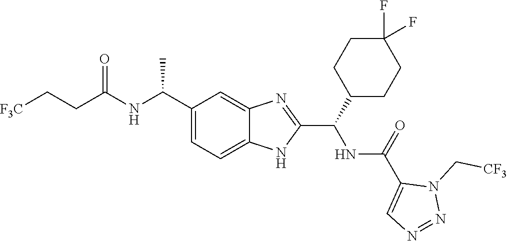 [SMILES: C[C@@H](NC(=O)CCC(F)(F)F)c1ccc2[nH]c([C@@H](NC(=O)c3cnnn3CC(F)(F)F)C3CCC(F)(F)CC3)nc2c1]

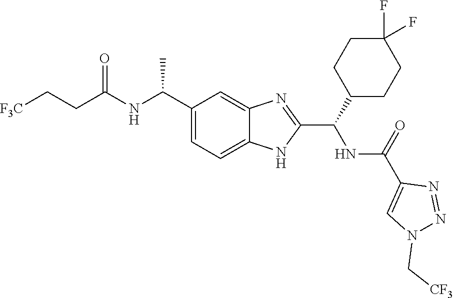 [SMILES: C[C@@H](NC(=O)CCC(F)(F)F)c1ccc2[nH]c([C@@H](NC(=O)c3cn(CC(F)(F)F)nn3)C3CCC(F)(F)CC3)nc2c1]

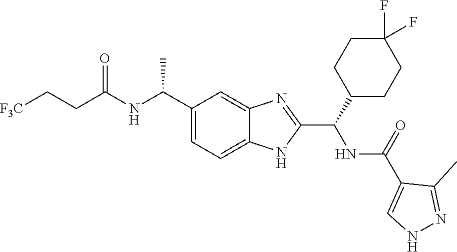 [SMILES: Cc1n[nH]cc1C(=O)N[C@H](c1nc2cc([C@@H](C)NC(=O)CCC(F)(F)F)ccc2[nH]1)C1CCC(F)(F)CC1]

TABLE 1B

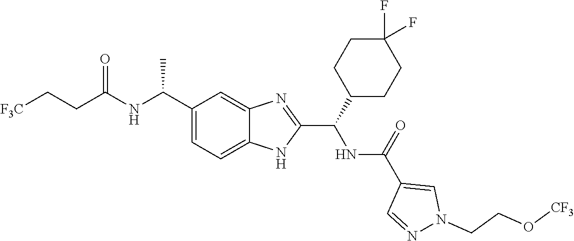 [SMILES: C[C@@H](NC(=O)CCC(F)(F)F)c1ccc2[nH]c([C@@H](NC(=O)c3cnn(CCOC(F)(F)F)c3)C3CCC(F)(F)CC3)nc2c1]

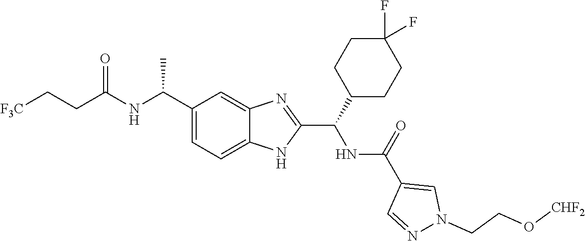 [SMILES: C[C@@H](NC(=O)CCC(F)(F)F)c1ccc2[nH]c([C@@H](NC(=O)c3cnn(CCOC(F)F)c3)C3CCC(F)(F)CC3)nc2c1]

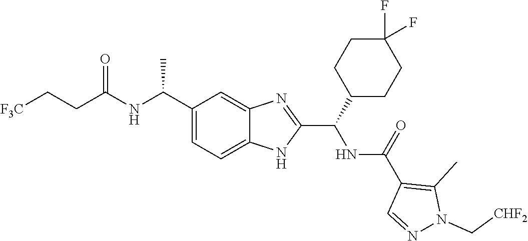 [SMILES: Cc1c(C(=O)N[C@H](c2nc3cc([C@@H](C)NC(=O)CCC(F)(F)F)ccc3[nH]2)C2CCC(F)(F)CC2)cnn1CC(F)F]

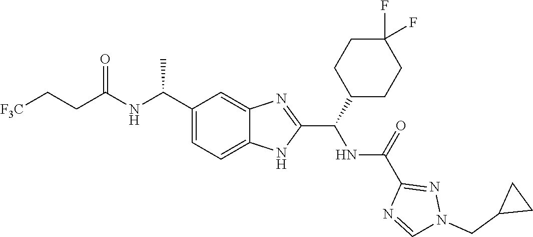 [SMILES: C[C@@H](NC(=O)CCC(F)(F)F)c1ccc2[nH]c([C@@H](NC(=O)c3ncn(CC4CC4)n3)C3CCC(F)(F)CC3)nc2c1]

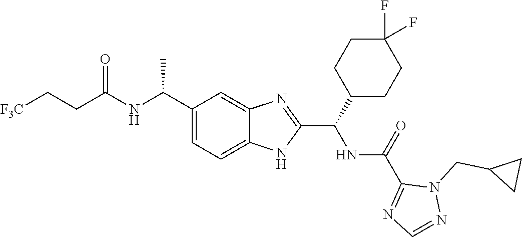 [SMILES: C[C@@H](NC(=O)CCC(F)(F)F)c1ccc2[nH]c([C@@H](NC(=O)c3ncnn3CC3CC3)C3CCC(F)(F)CC3)nc2c1]

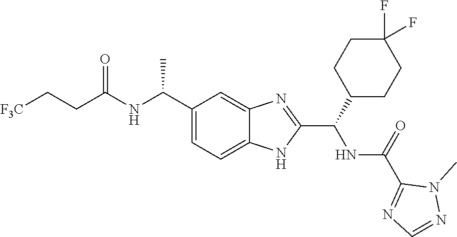 [SMILES: C[C@@H](NC(=O)CCC(F)(F)F)c1ccc2[nH]c([C@@H](NC(=O)c3ncnn3C)C3CCC(F)(F)CC3)nc2c1]

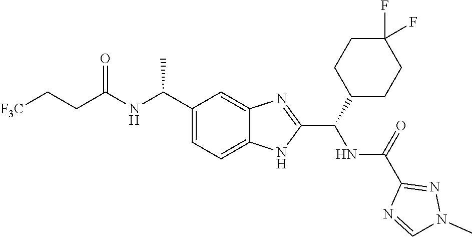 [SMILES: C[C@@H](NC(=O)CCC(F)(F)F)c1ccc2[nH]c([C@@H](NC(=O)c3ncn(C)n3)C3CCC(F)(F)CC3)nc2c1]

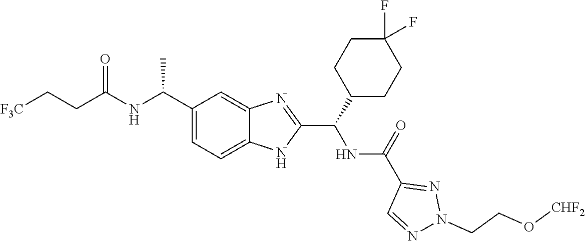 [SMILES: C[C@@H](NC(=O)CCC(F)(F)F)c1ccc2[nH]c([C@@H](NC(=O)c3cnn(CCOC(F)F)n3)C3CCC(F)(F)CC3)nc2c1]

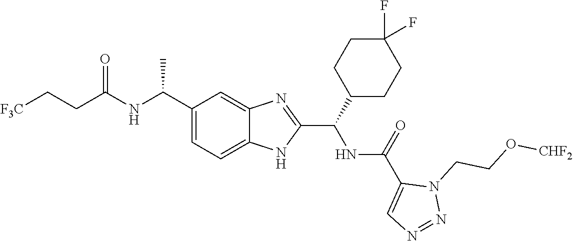 [SMILES: C[C@@H](NC(=O)CCC(F)(F)F)c1ccc2[nH]c([C@@H](NC(=O)c3cnnn3CCOC(F)F)C3CCC(F)(F)CC3)nc2c1]

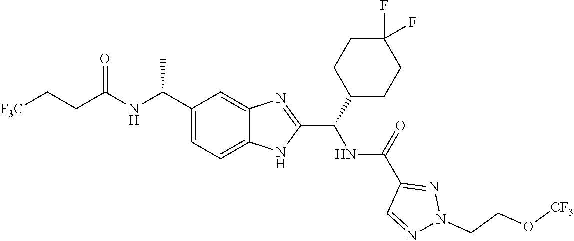 [SMILES: C[C@@H](NC(=O)CCC(F)(F)F)c1ccc2[nH]c([C@@H](NC(=O)c3cnn(CCOC(F)(F)F)n3)C3CCC(F)(F)CC3)nc2c1]

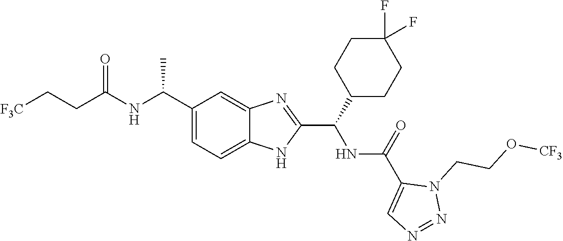 [SMILES: C[C@@H](NC(=O)CCC(F)(F)F)c1ccc2[nH]c([C@@H](NC(=O)c3cnnn3CCOC(F)(F)F)C3CCC(F)(F)CC3)nc2c1]

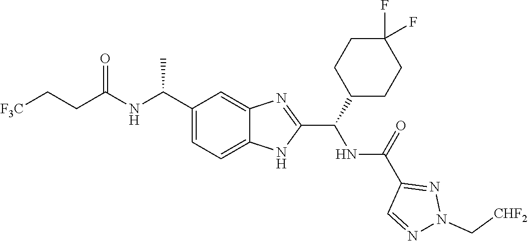 [SMILES: C[C@@H](NC(=O)CCC(F)(F)F)c1ccc2[nH]c([C@@H](NC(=O)c3cnn(CC(F)F)n3)C3CCC(F)(F)CC3)nc2c1]

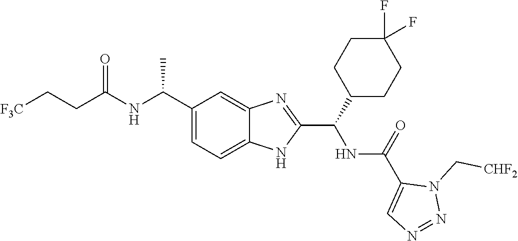 [SMILES: C[C@@H](NC(=O)CCC(F)(F)F)c1ccc2[nH]c([C@@H](NC(=O)c3cnnn3CC(F)F)C3CCC(F)(F)CC3)nc2c1]

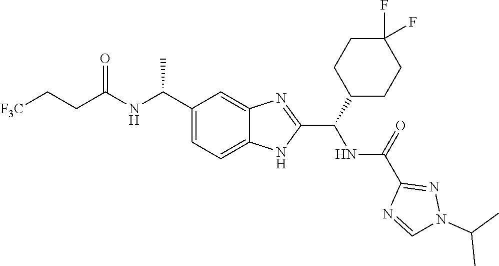 [SMILES: CC(C)n1cnc(C(=O)N[C@H](c2nc3cc([C@@H](C)NC(=O)CCC(F)(F)F)ccc3[nH]2)C2CCC(F)(F)CC2)n1]

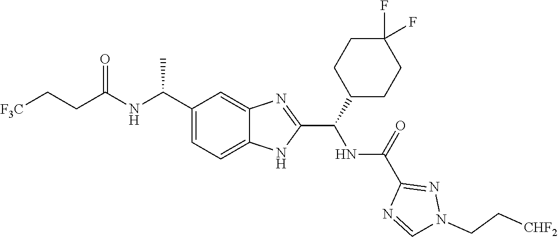 [SMILES: C[C@@H](NC(=O)CCC(F)(F)F)c1ccc2[nH]c([C@@H](NC(=O)c3ncn(CCC(F)F)n3)C3CCC(F)(F)CC3)nc2c1]

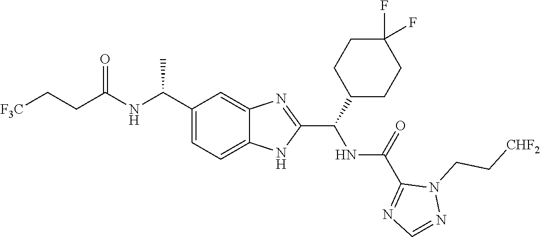 [SMILES: C[C@@H](NC(=O)CCC(F)(F)F)c1ccc2[nH]c([C@@H](NC(=O)c3ncnn3CCC(F)F)C3CCC(F)(F)CC3)nc2c1]

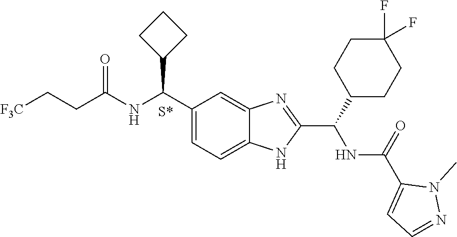 [SMILES: Cn1nccc1C(=O)N[C@H](c1nc2cc([C@@H](NC(=O)CCC(F)(F)F)C3CCC3)ccc2[nH]1)C1CCC(F)(F)CC1]

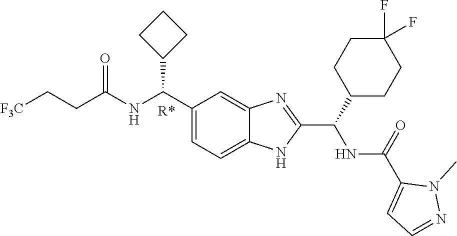 [SMILES: Cn1nccc1C(=O)N[C@H](c1nc2cc([C@H](NC(=O)CCC(F)(F)F)C3CCC3)ccc2[nH]1)C1CCC(F)(F)CC1]

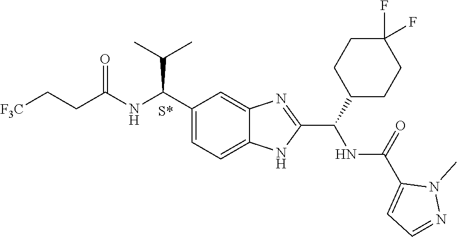 [SMILES: CC(C)[C@H](NC(=O)CCC(F)(F)F)c1ccc2[nH]c([C@@H](NC(=O)c3ccnn3C)C3CCC(F)(F)CC3)nc2c1]

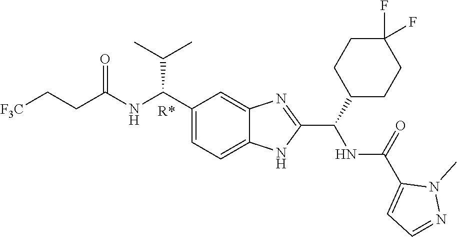 [SMILES: CC(C)[C@@H](NC(=O)CCC(F)(F)F)c1ccc2[nH]c([C@@H](NC(=O)c3ccnn3C)C3CCC(F)(F)CC3)nc2c1]

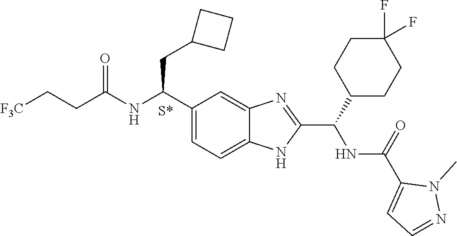 [SMILES: Cn1nccc1C(=O)N[C@H](c1nc2cc([C@H](CC3CCC3)NC(=O)CCC(F)(F)F)ccc2[nH]1)C1CCC(F)(F)CC1]

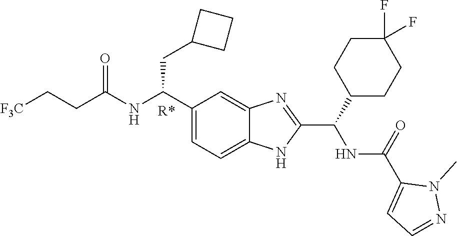 [SMILES: Cn1nccc1C(=O)N[C@H](c1nc2cc([C@@H](CC3CCC3)NC(=O)CCC(F)(F)F)ccc2[nH]1)C1CCC(F)(F)CC1]

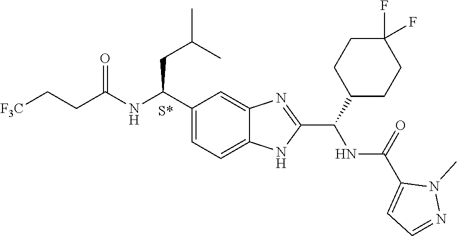 [SMILES: CC(C)C[C@H](NC(=O)CCC(F)(F)F)c1ccc2[nH]c([C@@H](NC(=O)c3ccnn3C)C3CCC(F)(F)CC3)nc2c1]

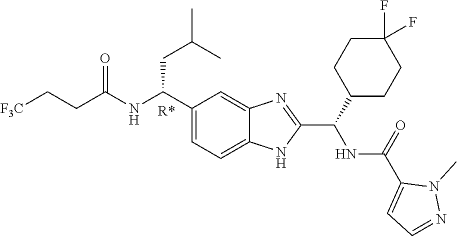 [SMILES: CC(C)C[C@@H](NC(=O)CCC(F)(F)F)c1ccc2[nH]c([C@@H](NC(=O)c3ccnn3C)C3CCC(F)(F)CC3)nc2c1]

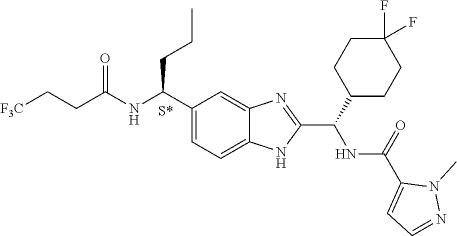 [SMILES: CCC[C@H](NC(=O)CCC(F)(F)F)c1ccc2[nH]c([C@@H](NC(=O)c3ccnn3C)C3CCC(F)(F)CC3)nc2c1]

TABLE 1C

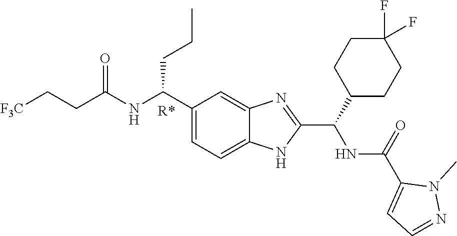 [SMILES: CCC[C@@H](NC(=O)CCC(F)(F)F)c1ccc2[nH]c([C@@H](NC(=O)c3ccnn3C)C3CCC(F)(F)CC3)nc2c1]

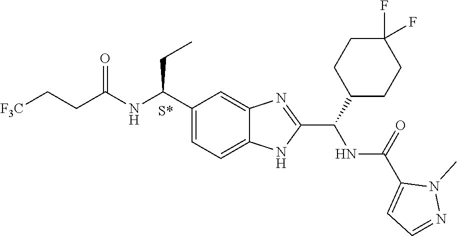 [SMILES: CC[C@H](NC(=O)CCC(F)(F)F)c1ccc2[nH]c([C@@H](NC(=O)c3ccnn3C)C3CCC(F)(F)CC3)nc2c1]

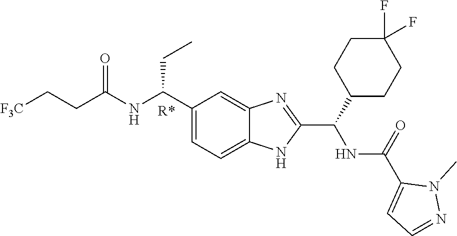 [SMILES: CC[C@@H](NC(=O)CCC(F)(F)F)c1ccc2[nH]c([C@@H](NC(=O)c3ccnn3C)C3CCC(F)(F)CC3)nc2c1]

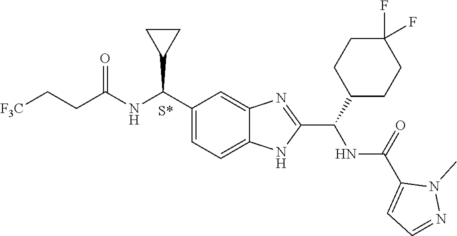 [SMILES: Cn1nccc1C(=O)N[C@H](c1nc2cc([C@@H](NC(=O)CCC(F)(F)F)C3CC3)ccc2[nH]1)C1CCC(F)(F)CC1]

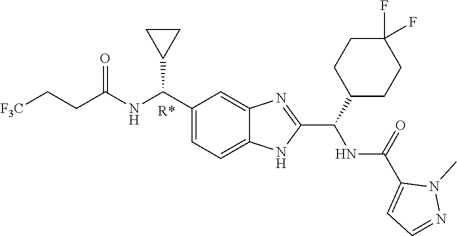 [SMILES: Cn1nccc1C(=O)N[C@H](c1nc2cc([C@H](NC(=O)CCC(F)(F)F)C3CC3)ccc2[nH]1)C1CCC(F)(F)CC1]

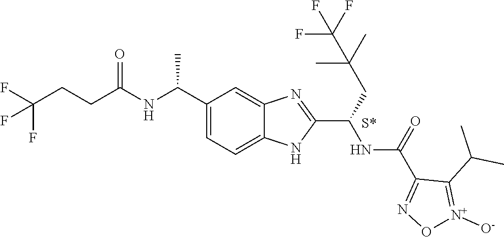 [SMILES: CC(C)c1c(C(=O)N[C@@H](CC(C)(C)C(F)(F)F)c2nc3cc([C@@H](C)NC(=O)CCC(F)(F)F)ccc3[nH]2)no[n+]1[O-]]

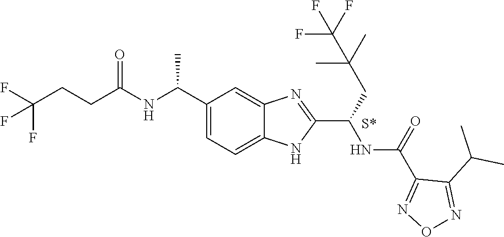 [SMILES: CC(C)c1nonc1C(=O)N[C@@H](CC(C)(C)C(F)(F)F)c1nc2cc([C@@H](C)NC(=O)CCC(F)(F)F)ccc2[nH]1]

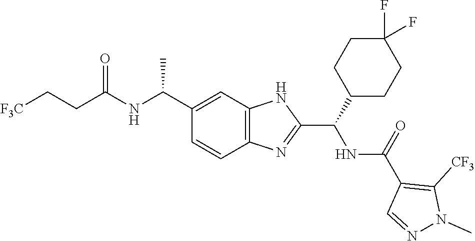 [SMILES: C[C@@H](NC(=O)CCC(F)(F)F)c1ccc2nc([C@@H](NC(=O)c3cnn(C)c3C(F)(F)F)C3CCC(F)(F)CC3)[nH]c2c1]

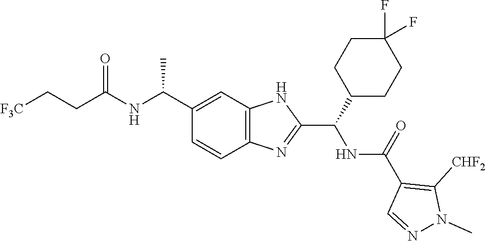 [SMILES: C[C@@H](NC(=O)CCC(F)(F)F)c1ccc2nc([C@@H](NC(=O)c3cnn(C)c3C(F)F)C3CCC(F)(F)CC3)[nH]c2c1]

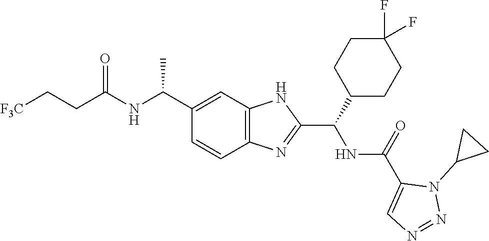 [SMILES: C[C@@H](NC(=O)CCC(F)(F)F)c1ccc2nc([C@@H](NC(=O)c3cnnn3C3CC3)C3CCC(F)(F)CC3)[nH]c2c1]

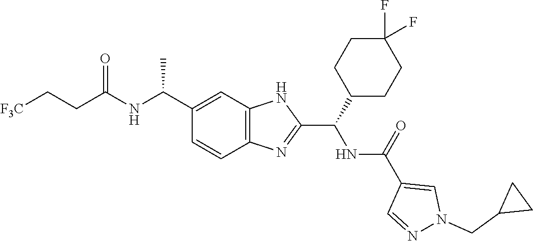 [SMILES: C[C@@H](NC(=O)CCC(F)(F)F)c1ccc2nc([C@@H](NC(=O)c3cnn(CC4CC4)c3)C3CCC(F)(F)CC3)[nH]c2c1]

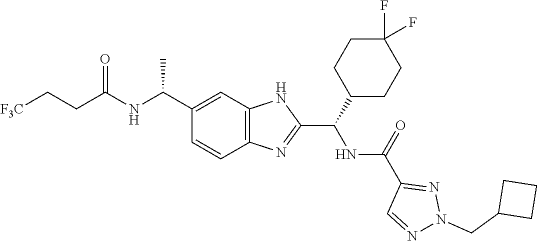 [SMILES: C[C@@H](NC(=O)CCC(F)(F)F)c1ccc2nc([C@@H](NC(=O)c3cnn(CC4CCC4)n3)C3CCC(F)(F)CC3)[nH]c2c1]

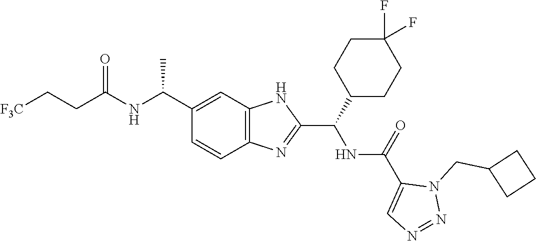 [SMILES: C[C@@H](NC(=O)CCC(F)(F)F)c1ccc2nc([C@@H](NC(=O)c3cnnn3CC3CCC3)C3CCC(F)(F)CC3)[nH]c2c1]

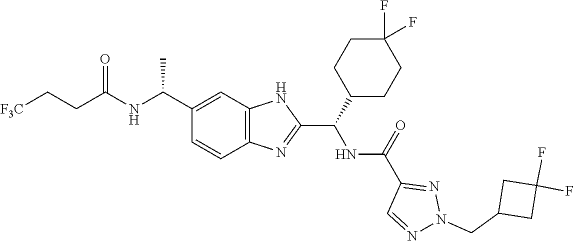 [SMILES: C[C@@H](NC(=O)CCC(F)(F)F)c1ccc2nc([C@@H](NC(=O)c3cnn(CC4CC(F)(F)C4)n3)C3CCC(F)(F)CC3)[nH]c2c1]

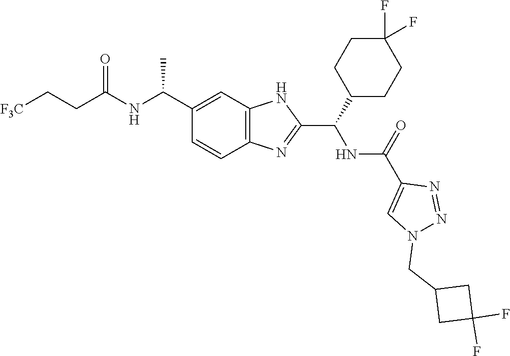 [SMILES: C[C@@H](NC(=O)CCC(F)(F)F)c1ccc2nc([C@@H](NC(=O)c3cn(CC4CC(F)(F)C4)nn3)C3CCC(F)(F)CC3)[nH]c2c1]

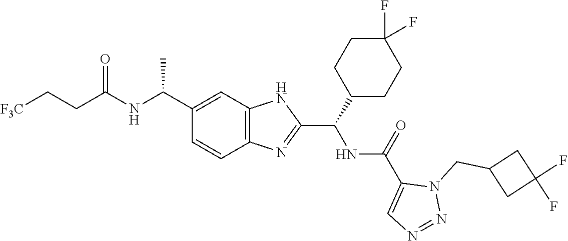 [SMILES: C[C@@H](NC(=O)CCC(F)(F)F)c1ccc2nc([C@@H](NC(=O)c3cnnn3CC3CC(F)(F)C3)C3CCC(F)(F)CC3)[nH]c2c1]

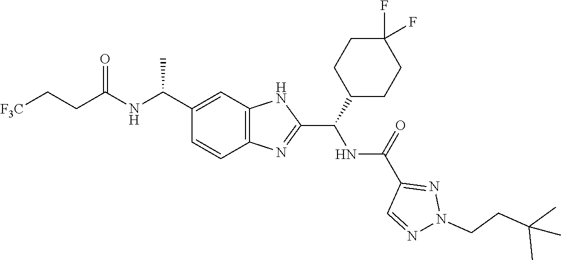 [SMILES: C[C@@H](NC(=O)CCC(F)(F)F)c1ccc2nc([C@@H](NC(=O)c3cnn(CCC(C)(C)C)n3)C3CCC(F)(F)CC3)[nH]c2c1]

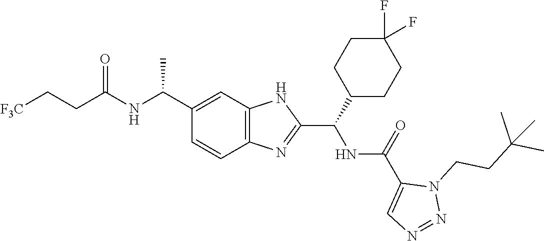 [SMILES: C[C@@H](NC(=O)CCC(F)(F)F)c1ccc2nc([C@@H](NC(=O)c3cnnn3CCC(C)(C)C)C3CCC(F)(F)CC3)[nH]c2c1]

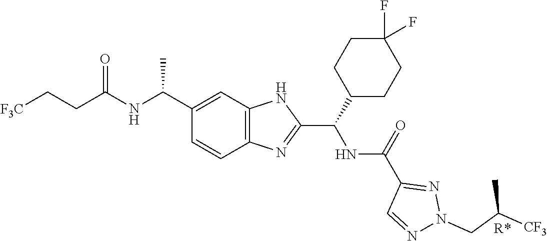 [SMILES: C[C@H](Cn1ncc(C(=O)N[C@H](c2nc3ccc([C@@H](C)NC(=O)CCC(F)(F)F)cc3[nH]2)C2CCC(F)(F)CC2)n1)C(F)(F)F]

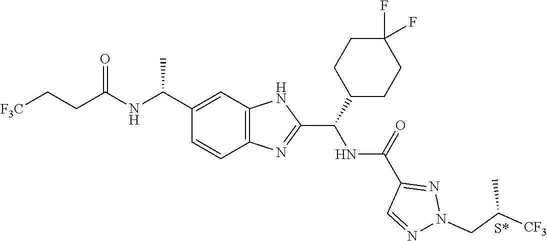 [SMILES: C[C@@H](NC(=O)CCC(F)(F)F)c1ccc2nc([C@@H](NC(=O)c3cnn(C[C@H](C)C(F)(F)F)n3)C3CCC(F)(F)CC3)[nH]c2c1]

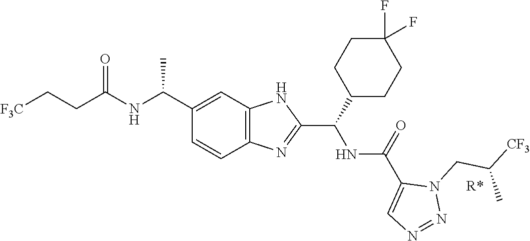 [SMILES: C[C@H](Cn1nncc1C(=O)N[C@H](c1nc2ccc([C@@H](C)NC(=O)CCC(F)(F)F)cc2[nH]1)C1CCC(F)(F)CC1)C(F)(F)F]

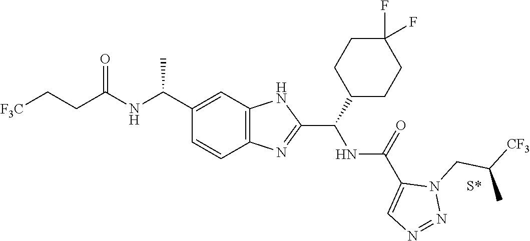 [SMILES: C[C@@H](NC(=O)CCC(F)(F)F)c1ccc2nc([C@@H](NC(=O)c3cnnn3C[C@H](C)C(F)(F)F)C3CCC(F)(F)CC3)[nH]c2c1]

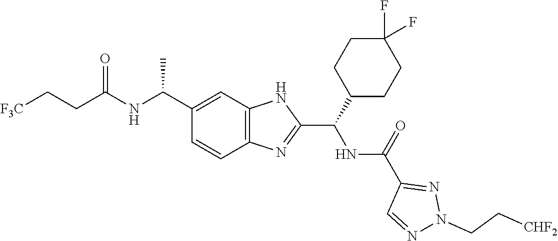 [SMILES: C[C@@H](NC(=O)CCC(F)(F)F)c1ccc2nc([C@@H](NC(=O)c3cnn(CCC(F)F)n3)C3CCC(F)(F)CC3)[nH]c2c1]

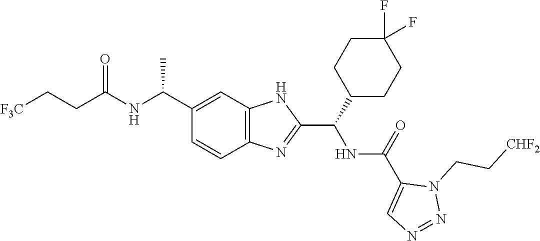 [SMILES: C[C@@H](NC(=O)CCC(F)(F)F)c1ccc2nc([C@@H](NC(=O)c3cnnn3CCC(F)F)C3CCC(F)(F)CC3)[nH]c2c1]

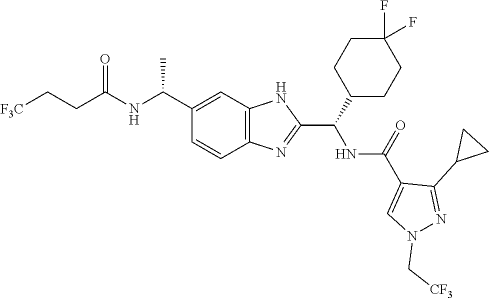 [SMILES: C[C@@H](NC(=O)CCC(F)(F)F)c1ccc2nc([C@@H](NC(=O)c3cn(CC(F)(F)F)nc3C3CC3)C3CCC(F)(F)CC3)[nH]c2c1]

TABLE 1D

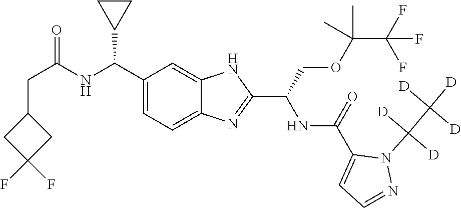 [SMILES: [2H]C([2H])([2H])C([2H])([2H])n1nccc1C(=O)N[C@@H](COC(C)(C)C(F)(F)F)c1nc2ccc([C@H](NC(=O)CC3CC(F)(F)C3)C3CC3)cc2[nH]1]

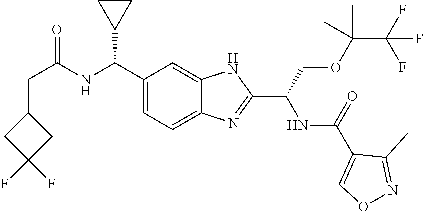 [SMILES: Cc1nocc1C(=O)N[C@@H](COC(C)(C)C(F)(F)F)c1nc2ccc([C@H](NC(=O)CC3CC(F)(F)C3)C3CC3)cc2[nH]1]

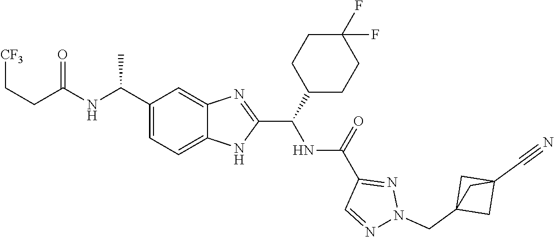 [SMILES: C[C@@H](NC(=O)CCC(F)(F)F)c1ccc2[nH]c([C@@H](NC(=O)c3cnn(CC45CC(C#N)(C4)C5)n3)C3CCC(F)(F)CC3)nc2c1]

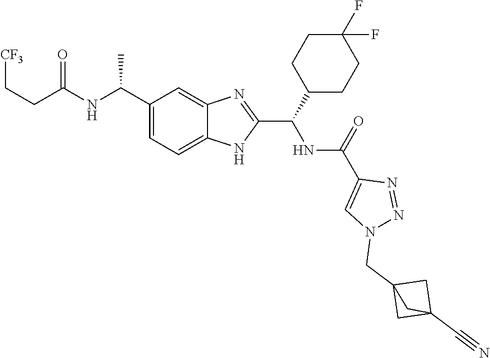 [SMILES: C[C@@H](NC(=O)CCC(F)(F)F)c1ccc2[nH]c([C@@H](NC(=O)c3cn(CC45CC(C#N)(C4)C5)nn3)C3CCC(F)(F)CC3)nc2c1]

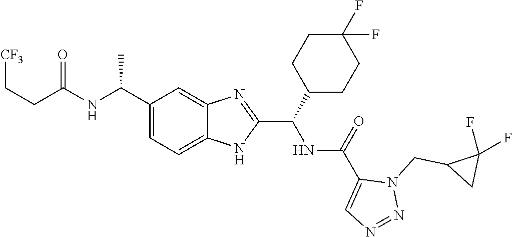 [SMILES: C[C@@H](NC(=O)CCC(F)(F)F)c1ccc2[nH]c([C@@H](NC(=O)c3cnnn3CC3CC3(F)F)C3CCC(F)(F)CC3)nc2c1]

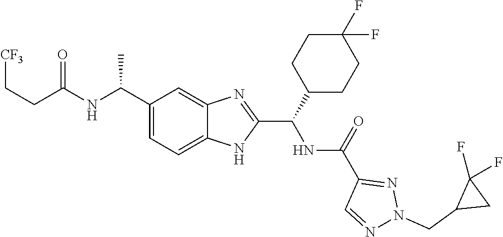 [SMILES: C[C@@H](NC(=O)CCC(F)(F)F)c1ccc2[nH]c([C@@H](NC(=O)c3cnn(CC4CC4(F)F)n3)C3CCC(F)(F)CC3)nc2c1]

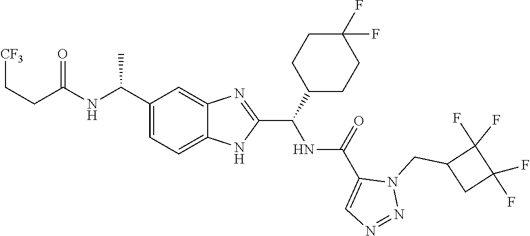 [SMILES: C[C@@H](NC(=O)CCC(F)(F)F)c1ccc2[nH]c([C@@H](NC(=O)c3cnnn3CC3CC(F)(F)C3(F)F)C3CCC(F)(F)CC3)nc2c1]

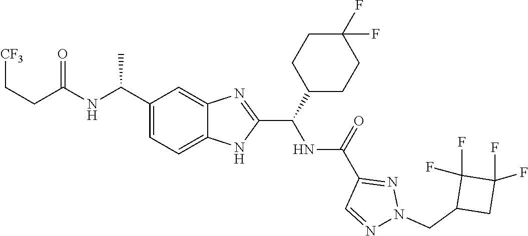 [SMILES: C[C@@H](NC(=O)CCC(F)(F)F)c1ccc2[nH]c([C@@H](NC(=O)c3cnn(CC4CC(F)(F)C4(F)F)n3)C3CCC(F)(F)CC3)nc2c1]

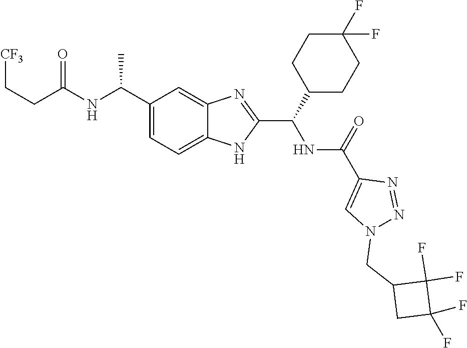 [SMILES: C[C@@H](NC(=O)CCC(F)(F)F)c1ccc2[nH]c([C@@H](NC(=O)c3cn(CC4CC(F)(F)C4(F)F)nn3)C3CCC(F)(F)CC3)nc2c1]

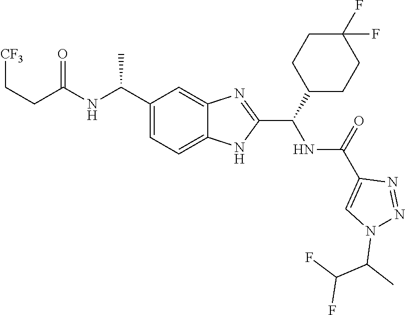 [SMILES: CC(C(F)F)n1cc(C(=O)N[C@H](c2nc3cc([C@@H](C)NC(=O)CCC(F)(F)F)ccc3[nH]2)C2CCC(F)(F)CC2)nn1]

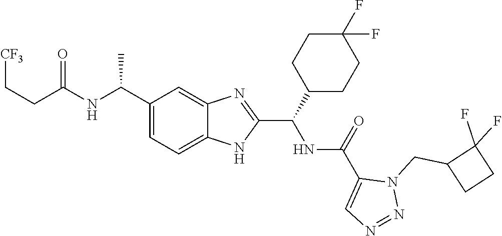 [SMILES: C[C@@H](NC(=O)CCC(F)(F)F)c1ccc2[nH]c([C@@H](NC(=O)c3cnnn3CC3CCC3(F)F)C3CCC(F)(F)CC3)nc2c1]

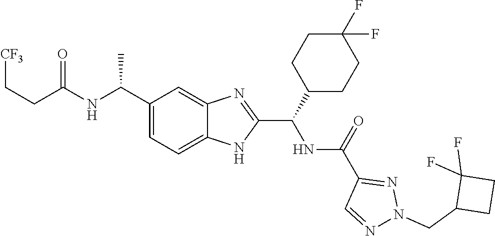 [SMILES: C[C@@H](NC(=O)CCC(F)(F)F)c1ccc2[nH]c([C@@H](NC(=O)c3cnn(CC4CCC4(F)F)n3)C3CCC(F)(F)CC3)nc2c1]

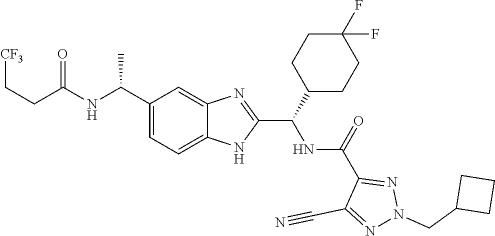 [SMILES: C[C@@H](NC(=O)CCC(F)(F)F)c1ccc2[nH]c([C@@H](NC(=O)c3nn(CC4CCC4)nc3C#N)C3CCC(F)(F)CC3)nc2c1]

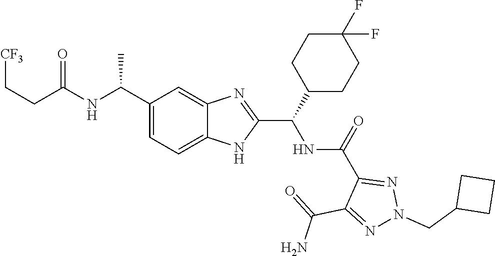 [SMILES: C[C@@H](NC(=O)CCC(F)(F)F)c1ccc2[nH]c([C@@H](NC(=O)c3nn(CC4CCC4)nc3C(N)=O)C3CCC(F)(F)CC3)nc2c1]

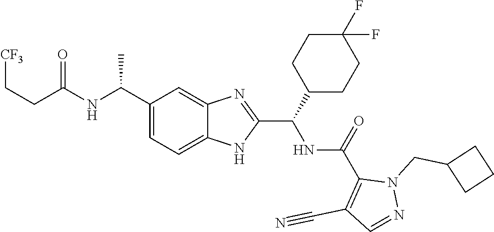 [SMILES: C[C@@H](NC(=O)CCC(F)(F)F)c1ccc2[nH]c([C@@H](NC(=O)c3c(C#N)cnn3CC3CCC3)C3CCC(F)(F)CC3)nc2c1]

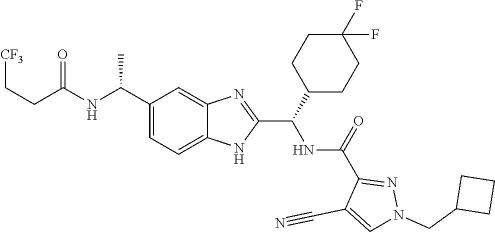 [SMILES: C[C@@H](NC(=O)CCC(F)(F)F)c1ccc2[nH]c([C@@H](NC(=O)c3nn(CC4CCC4)cc3C#N)C3CCC(F)(F)CC3)nc2c1]

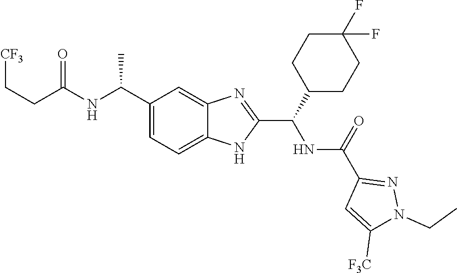 [SMILES: CCn1nc(C(=O)N[C@H](c2nc3cc([C@@H](C)NC(=O)CCC(F)(F)F)ccc3[nH]2)C2CCC(F)(F)CC2)cc1C(F)(F)F]

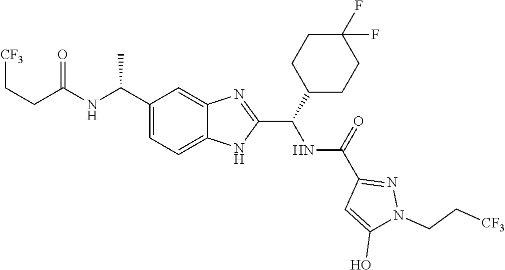 [SMILES: C[C@@H](NC(=O)CCC(F)(F)F)c1ccc2[nH]c([C@@H](NC(=O)c3cc(O)n(CCC(F)(F)F)n3)C3CCC(F)(F)CC3)nc2c1]

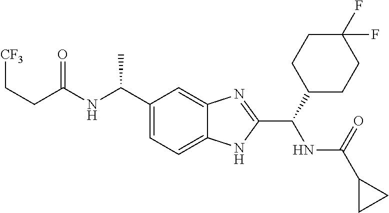 [SMILES: C[C@@H](NC(=O)CCC(F)(F)F)c1ccc2[nH]c([C@@H](NC(=O)C3CC3)C3CCC(F)(F)CC3)nc2c1]

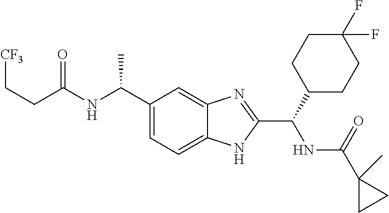 [SMILES: C[C@@H](NC(=O)CCC(F)(F)F)c1ccc2[nH]c([C@@H](NC(=O)C3(C)CC3)C3CCC(F)(F)CC3)nc2c1]

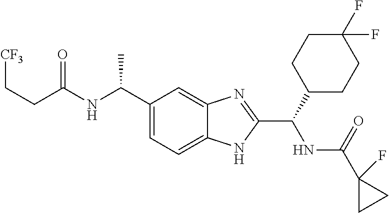 [SMILES: C[C@@H](NC(=O)CCC(F)(F)F)c1ccc2[nH]c([C@@H](NC(=O)C3(F)CC3)C3CCC(F)(F)CC3)nc2c1]

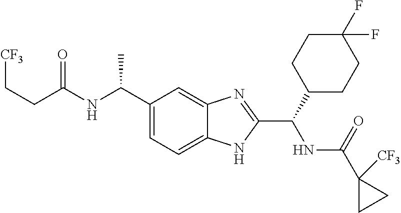 [SMILES: C[C@@H](NC(=O)CCC(F)(F)F)c1ccc2[nH]c([C@@H](NC(=O)C3(C(F)(F)F)CC3)C3CCC(F)(F)CC3)nc2c1]

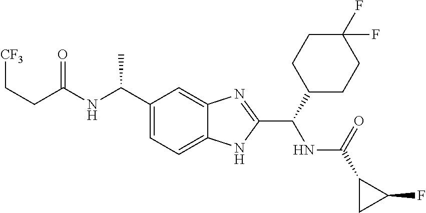 [SMILES: C[C@@H](NC(=O)CCC(F)(F)F)c1ccc2[nH]c([C@@H](NC(=O)[C@H]3C[C@@H]3F)C3CCC(F)(F)CC3)nc2c1]

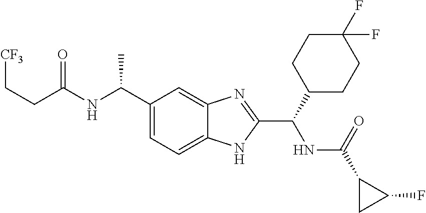 [SMILES: C[C@@H](NC(=O)CCC(F)(F)F)c1ccc2[nH]c([C@@H](NC(=O)[C@H]3C[C@H]3F)C3CCC(F)(F)CC3)nc2c1]

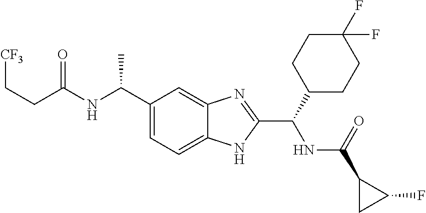 [SMILES: C[C@@H](NC(=O)CCC(F)(F)F)c1ccc2[nH]c([C@@H](NC(=O)[C@@H]3C[C@H]3F)C3CCC(F)(F)CC3)nc2c1]

TABLE 1E

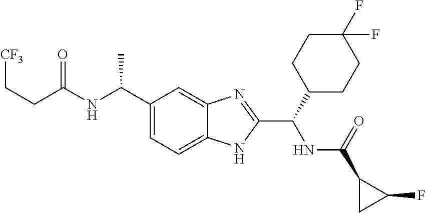 [SMILES: C[C@@H](NC(=O)CCC(F)(F)F)c1ccc2[nH]c([C@@H](NC(=O)[C@@H]3C[C@@H]3F)C3CCC(F)(F)CC3)nc2c1]

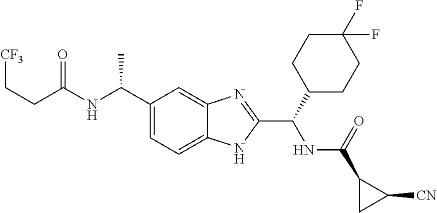 [SMILES: C[C@@H](NC(=O)CCC(F)(F)F)c1ccc2[nH]c([C@@H](NC(=O)[C@@H]3C[C@@H]3C#N)C3CCC(F)(F)CC3)nc2c1]

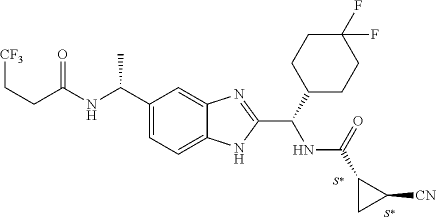 [SMILES: C[C@@H](NC(=O)CCC(F)(F)F)c1ccc2[nH]c([C@@H](NC(=O)[C@H]3C[C@@H]3C#N)C3CCC(F)(F)CC3)nc2c1]

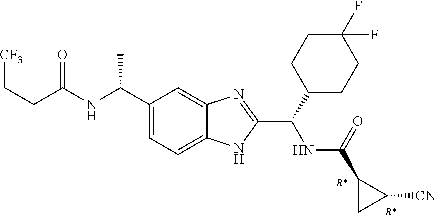 [SMILES: C[C@@H](NC(=O)CCC(F)(F)F)c1ccc2[nH]c([C@@H](NC(=O)[C@@H]3C[C@H]3C#N)C3CCC(F)(F)CC3)nc2c1]

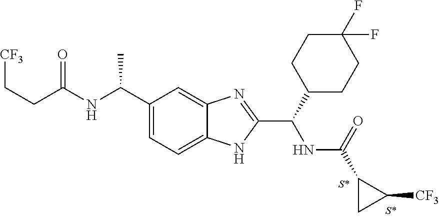 [SMILES: C[C@@H](NC(=O)CCC(F)(F)F)c1ccc2[nH]c([C@@H](NC(=O)[C@H]3C[C@@H]3C(F)(F)F)C3CCC(F)(F)CC3)nc2c1]

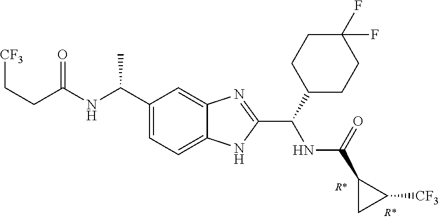 [SMILES: C[C@@H](NC(=O)CCC(F)(F)F)c1ccc2[nH]c([C@@H](NC(=O)[C@@H]3C[C@H]3C(F)(F)F)C3CCC(F)(F)CC3)nc2c1]

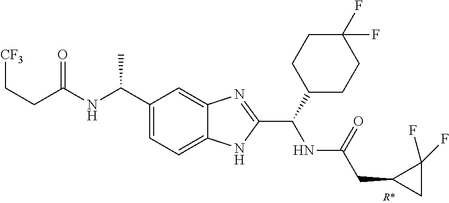 [SMILES: C[C@@H](NC(=O)CCC(F)(F)F)c1ccc2[nH]c([C@@H](NC(=O)C[C@@H]3CC3(F)F)C3CCC(F)(F)CC3)nc2c1]

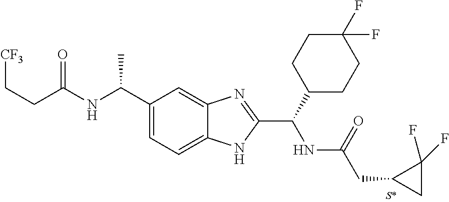 [SMILES: C[C@@H](NC(=O)CCC(F)(F)F)c1ccc2[nH]c([C@@H](NC(=O)C[C@H]3CC3(F)F)C3CCC(F)(F)CC3)nc2c1]

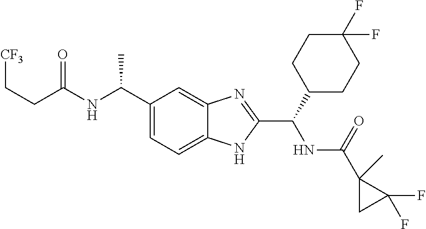 [SMILES: C[C@@H](NC(=O)CCC(F)(F)F)c1ccc2[nH]c([C@@H](NC(=O)C3(C)CC3(F)F)C3CCC(F)(F)CC3)nc2c1]

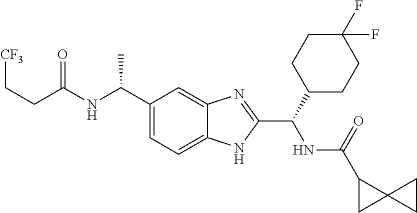 [SMILES: C[C@@H](NC(=O)CCC(F)(F)F)c1ccc2[nH]c([C@@H](NC(=O)C3CC34CC4)C3CCC(F)(F)CC3)nc2c1]

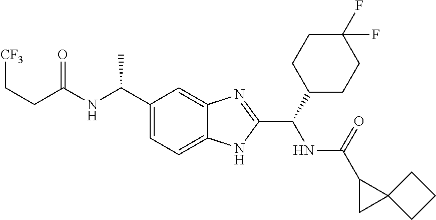 [SMILES: C[C@@H](NC(=O)CCC(F)(F)F)c1ccc2[nH]c([C@@H](NC(=O)C3CC34CCC4)C3CCC(F)(F)CC3)nc2c1]

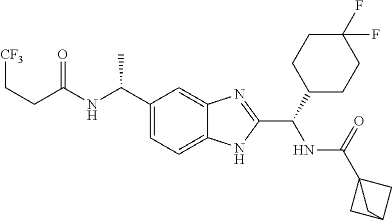 [SMILES: C[C@@H](NC(=O)CCC(F)(F)F)c1ccc2[nH]c([C@@H](NC(=O)C34CC(C3)C4)C3CCC(F)(F)CC3)nc2c1]

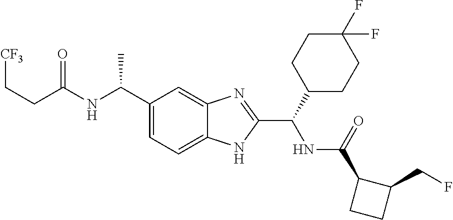 [SMILES: C[C@@H](NC(=O)CCC(F)(F)F)c1ccc2[nH]c([C@@H](NC(=O)[C@@H]3CC[C@@H]3CF)C3CCC(F)(F)CC3)nc2c1]

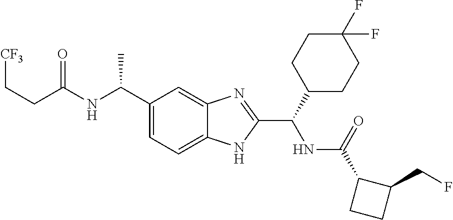 [SMILES: C[C@@H](NC(=O)CCC(F)(F)F)c1ccc2[nH]c([C@@H](NC(=O)[C@H]3CC[C@@H]3CF)C3CCC(F)(F)CC3)nc2c1]

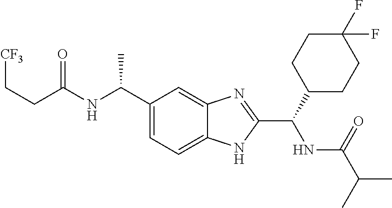 [SMILES: CC(C)C(=O)N[C@H](c1nc2cc([C@@H](C)NC(=O)CCC(F)(F)F)ccc2[nH]1)C1CCC(F)(F)CC1]

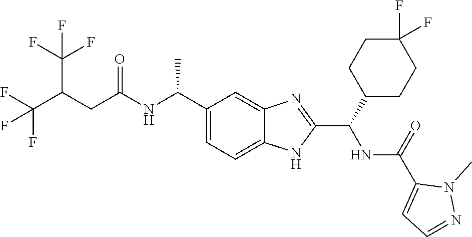 [SMILES: C[C@@H](NC(=O)CC(C(F)(F)F)C(F)(F)F)c1ccc2[nH]c([C@@H](NC(=O)c3ccnn3C)C3CCC(F)(F)CC3)nc2c1]

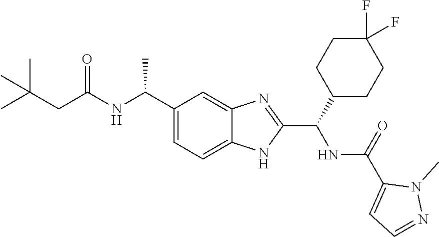 [SMILES: C[C@@H](NC(=O)CC(C)(C)C)c1ccc2[nH]c([C@@H](NC(=O)c3ccnn3C)C3CCC(F)(F)CC3)nc2c1]

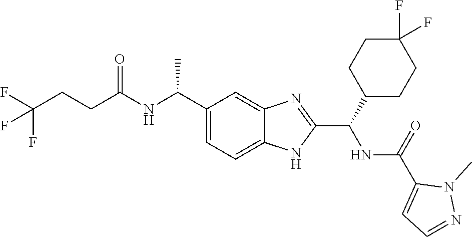 [SMILES: C[C@@H](NC(=O)CCC(F)(F)F)c1ccc2[nH]c([C@@H](NC(=O)c3ccnn3C)C3CCC(F)(F)CC3)nc2c1]

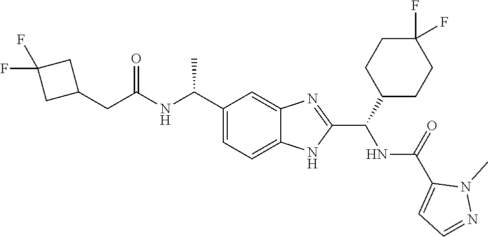 [SMILES: C[C@@H](NC(=O)CC1CC(F)(F)C1)c1ccc2[nH]c([C@@H](NC(=O)c3ccnn3C)C3CCC(F)(F)CC3)nc2c1]

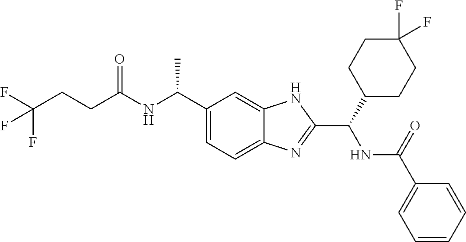 [SMILES: C[C@@H](NC(=O)CCC(F)(F)F)c1ccc2nc([C@@H](NC(=O)c3ccccc3)C3CCC(F)(F)CC3)[nH]c2c1]

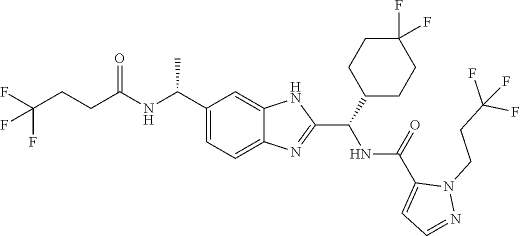 [SMILES: C[C@@H](NC(=O)CCC(F)(F)F)c1ccc2nc([C@@H](NC(=O)c3ccnn3CCC(F)(F)F)C3CCC(F)(F)CC3)[nH]c2c1]

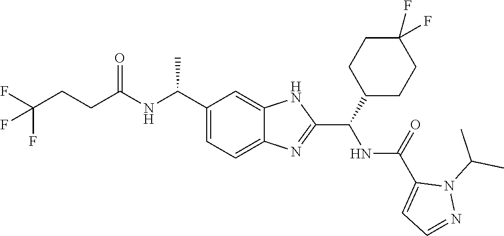 [SMILES: CC(C)n1nccc1C(=O)N[C@H](c1nc2ccc([C@@H](C)NC(=O)CCC(F)(F)F)cc2[nH]1)C1CCC(F)(F)CC1]

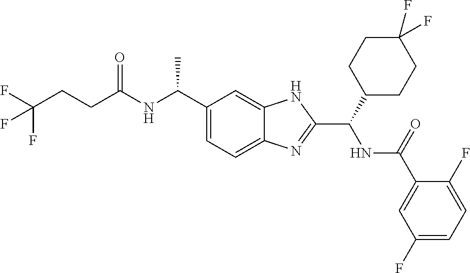 [SMILES: C[C@@H](NC(=O)CCC(F)(F)F)c1ccc2nc([C@@H](NC(=O)c3cc(F)ccc3F)C3CCC(F)(F)CC3)[nH]c2c1]

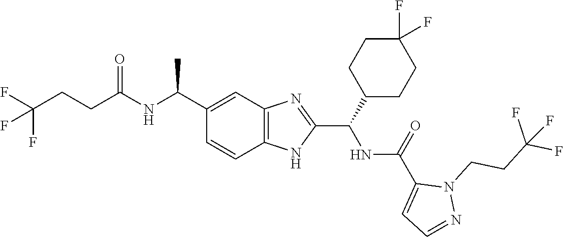 [SMILES: C[C@H](NC(=O)CCC(F)(F)F)c1ccc2[nH]c([C@@H](NC(=O)c3ccnn3CCC(F)(F)F)C3CCC(F)(F)CC3)nc2c1]

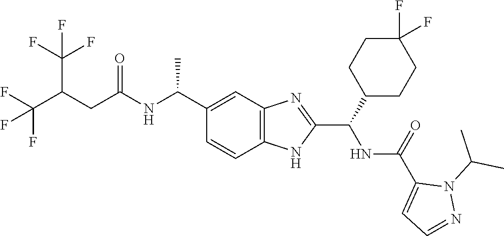 [SMILES: CC(C)n1nccc1C(=O)N[C@H](c1nc2cc([C@@H](C)NC(=O)CC(C(F)(F)F)C(F)(F)F)ccc2[nH]1)C1CCC(F)(F)CC1]

TABLE 1F

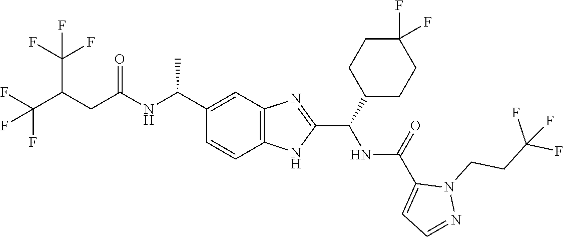 [SMILES: C[C@@H](NC(=O)CC(C(F)(F)F)C(F)(F)F)c1ccc2[nH]c([C@@H](NC(=O)c3ccnn3CCC(F)(F)F)C3CCC(F)(F)CC3)nc2c1]

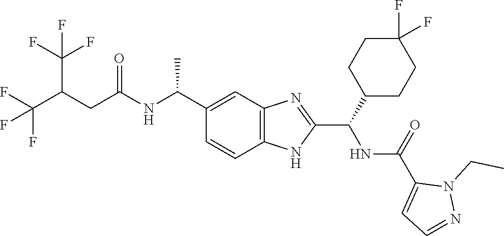 [SMILES: CCn1nccc1C(=O)N[C@H](c1nc2cc([C@@H](C)NC(=O)CC(C(F)(F)F)C(F)(F)F)ccc2[nH]1)C1CCC(F)(F)CC1]

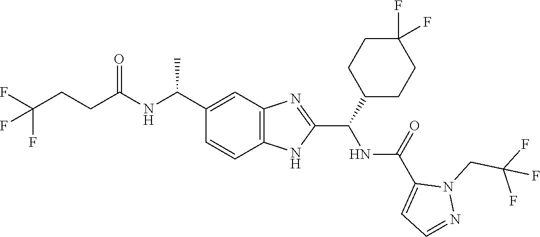 [SMILES: C[C@@H](NC(=O)CCC(F)(F)F)c1ccc2[nH]c([C@@H](NC(=O)c3ccnn3CC(F)(F)F)C3CCC(F)(F)CC3)nc2c1]

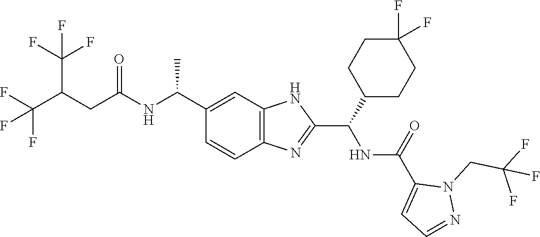 [SMILES: C[C@@H](NC(=O)CC(C(F)(F)F)C(F)(F)F)c1ccc2nc([C@@H](NC(=O)c3ccnn3CC(F)(F)F)C3CCC(F)(F)CC3)[nH]c2c1]

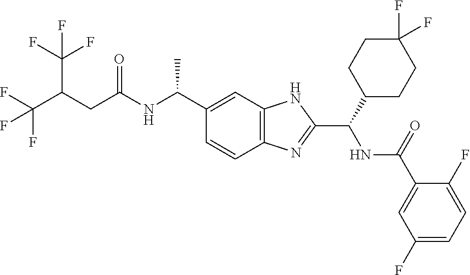 [SMILES: C[C@@H](NC(=O)CC(C(F)(F)F)C(F)(F)F)c1ccc2nc([C@@H](NC(=O)c3cc(F)ccc3F)C3CCC(F)(F)CC3)[nH]c2c1]

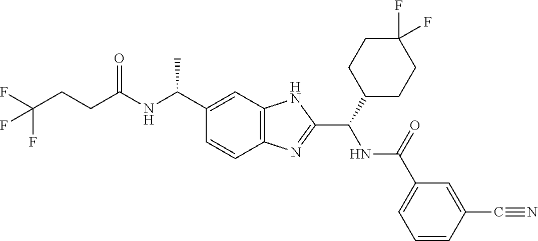 [SMILES: C[C@@H](NC(=O)CCC(F)(F)F)c1ccc2nc([C@@H](NC(=O)c3cccc(C#N)c3)C3CCC(F)(F)CC3)[nH]c2c1]

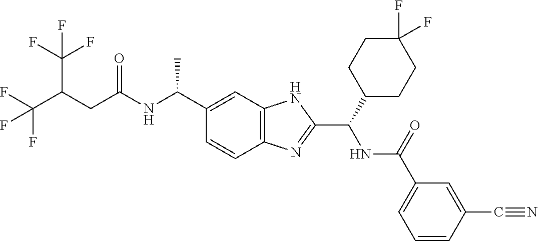 [SMILES: C[C@@H](NC(=O)CC(C(F)(F)F)C(F)(F)F)c1ccc2nc([C@@H](NC(=O)c3cccc(C#N)c3)C3CCC(F)(F)CC3)[nH]c2c1]

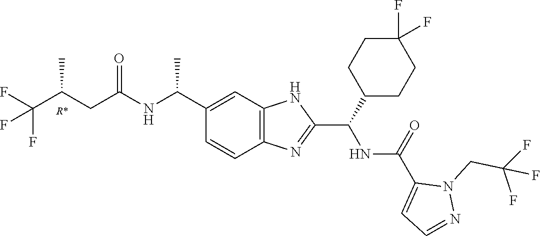 [SMILES: C[C@H](CC(=O)N[C@H](C)c1ccc2nc([C@@H](NC(=O)c3ccnn3CC(F)(F)F)C3CCC(F)(F)CC3)[nH]c2c1)C(F)(F)F]

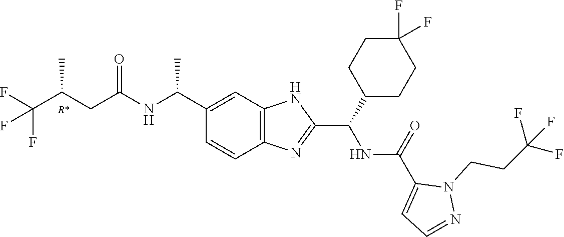 [SMILES: C[C@H](CC(=O)N[C@H](C)c1ccc2nc([C@@H](NC(=O)c3ccnn3CCC(F)(F)F)C3CCC(F)(F)CC3)[nH]c2c1)C(F)(F)F]

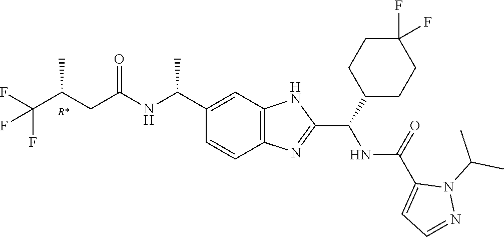 [SMILES: CC(C)n1nccc1C(=O)N[C@H](c1nc2ccc([C@@H](C)NC(=O)C[C@@H](C)C(F)(F)F)cc2[nH]1)C1CCC(F)(F)CC1]

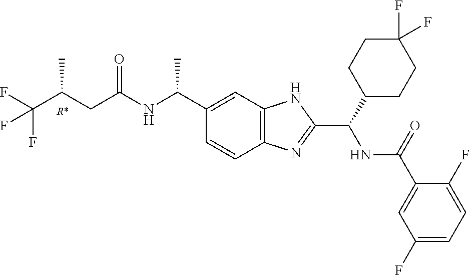 [SMILES: C[C@H](CC(=O)N[C@H](C)c1ccc2nc([C@@H](NC(=O)c3cc(F)ccc3F)C3CCC(F)(F)CC3)[nH]c2c1)C(F)(F)F]

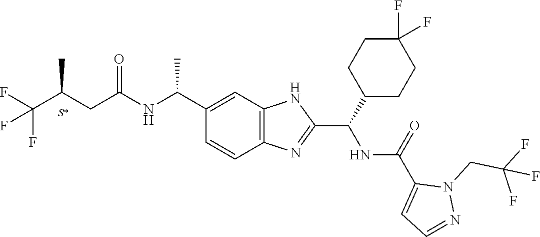 [SMILES: C[C@@H](NC(=O)C[C@H](C)C(F)(F)F)c1ccc2nc([C@@H](NC(=O)c3ccnn3CC(F)(F)F)C3CCC(F)(F)CC3)[nH]c2c1]

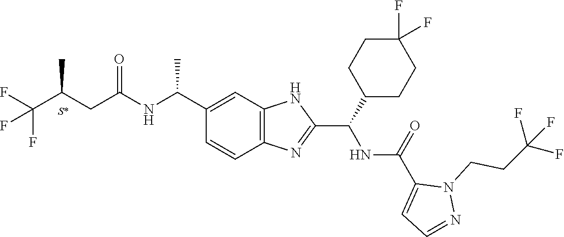 [SMILES: C[C@@H](NC(=O)C[C@H](C)C(F)(F)F)c1ccc2nc([C@@H](NC(=O)c3ccnn3CCC(F)(F)F)C3CCC(F)(F)CC3)[nH]c2c1]

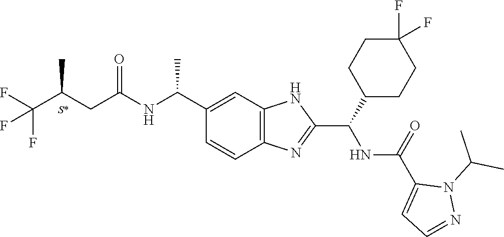 [SMILES: CC(C)n1nccc1C(=O)N[C@H](c1nc2ccc([C@@H](C)NC(=O)C[C@H](C)C(F)(F)F)cc2[nH]1)C1CCC(F)(F)CC1]

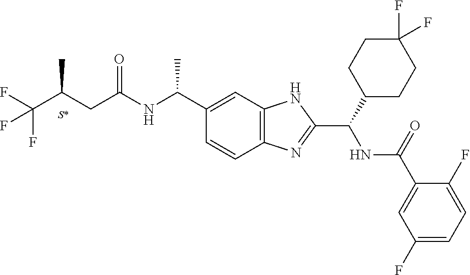 [SMILES: C[C@@H](NC(=O)C[C@H](C)C(F)(F)F)c1ccc2nc([C@@H](NC(=O)c3cc(F)ccc3F)C3CCC(F)(F)CC3)[nH]c2c1]

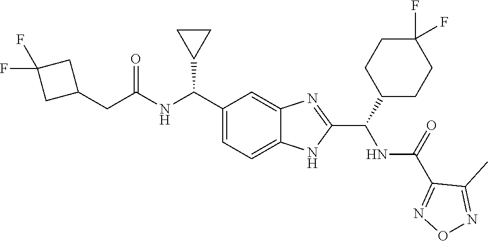 [SMILES: Cc1nonc1C(=O)N[C@H](c1nc2cc([C@H](NC(=O)CC3CC(F)(F)C3)C3CC3)ccc2[nH]1)C1CCC(F)(F)CC1]

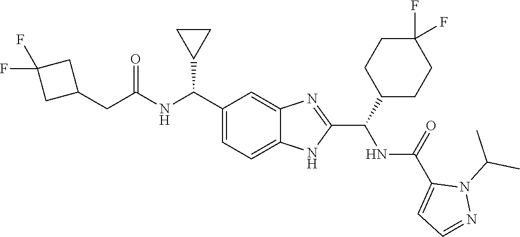 [SMILES: CC(C)n1nccc1C(=O)N[C@H](c1nc2cc([C@H](NC(=O)CC3CC(F)(F)C3)C3CC3)ccc2[nH]1)C1CCC(F)(F)CC1]

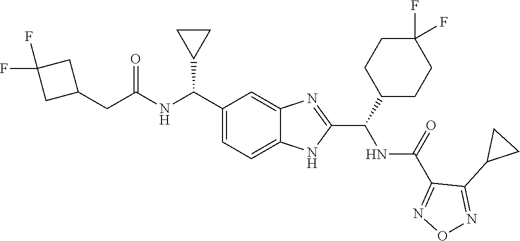 [SMILES: O=C(CC1CC(F)(F)C1)N[C@@H](c1ccc2[nH]c([C@@H](NC(=O)c3nonc3C3CC3)C3CCC(F)(F)CC3)nc2c1)C1CC1]

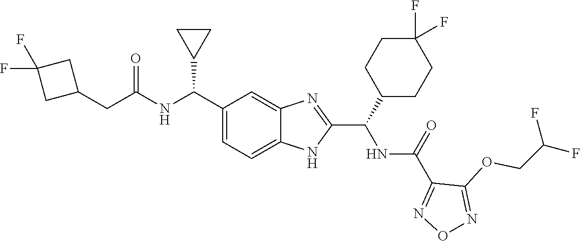 [SMILES: O=C(CC1CC(F)(F)C1)N[C@@H](c1ccc2[nH]c([C@@H](NC(=O)c3nonc3OCC(F)F)C3CCC(F)(F)CC3)nc2c1)C1CC1]

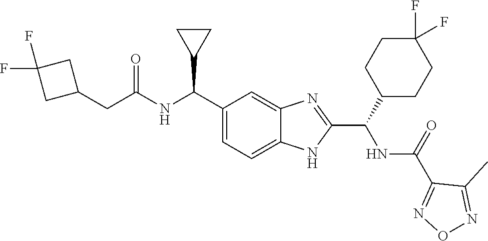 [SMILES: Cc1nonc1C(=O)N[C@H](c1nc2cc([C@@H](NC(=O)CC3CC(F)(F)C3)C3CC3)ccc2[nH]1)C1CCC(F)(F)CC1]

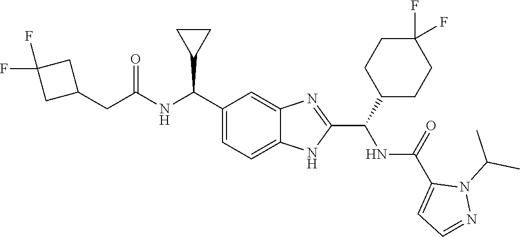 [SMILES: CC(C)n1nccc1C(=O)N[C@H](c1nc2cc([C@@H](NC(=O)CC3CC(F)(F)C3)C3CC3)ccc2[nH]1)C1CCC(F)(F)CC1]

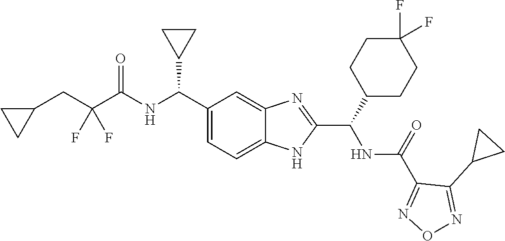 [SMILES: O=C(N[C@H](c1nc2cc([C@H](NC(=O)C(F)(F)CC3CC3)C3CC3)ccc2[nH]1)C1CCC(F)(F)CC1)c1nonc1C1CC1]

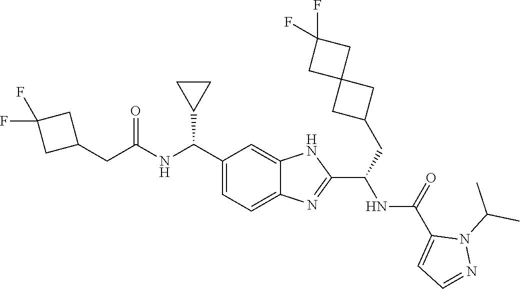 [SMILES: CC(C)n1nccc1C(=O)N[C@@H](CC1CC2(C1)CC(F)(F)C2)c1nc2ccc([C@H](NC(=O)CC3CC(F)(F)C3)C3CC3)cc2[nH]1]

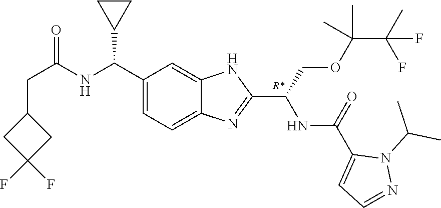 [SMILES: CC(C)n1nccc1C(=O)N[C@@H](COC(C)(C)C(C)(F)F)c1nc2ccc([C@H](NC(=O)CC3CC(F)(F)C3)C3CC3)cc2[nH]1]

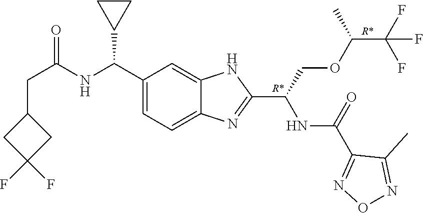 [SMILES: Cc1nonc1C(=O)N[C@@H](CO[C@H](C)C(F)(F)F)c1nc2ccc([C@H](NC(=O)CC3CC(F)(F)C3)C3CC3)cc2[nH]1]

TABLE 1G

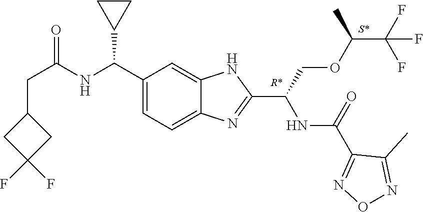 [SMILES: Cc1nonc1C(=O)N[C@@H](CO[C@@H](C)C(F)(F)F)c1nc2ccc([C@H](NC(=O)CC3CC(F)(F)C3)C3CC3)cc2[nH]1]

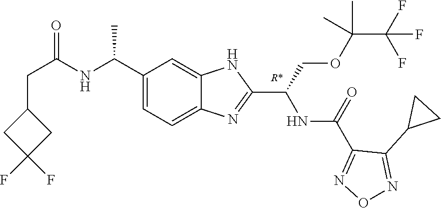 [SMILES: C[C@@H](NC(=O)CC1CC(F)(F)C1)c1ccc2nc([C@H](COC(C)(C)C(F)(F)F)NC(=O)c3nonc3C3CC3)[nH]c2c1]

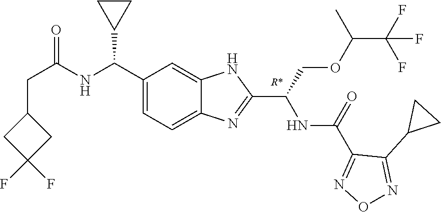 [SMILES: CC(OC[C@H](NC(=O)c1nonc1C1CC1)c1nc2ccc([C@H](NC(=O)CC3CC(F)(F)C3)C3CC3)cc2[nH]1)C(F)(F)F]

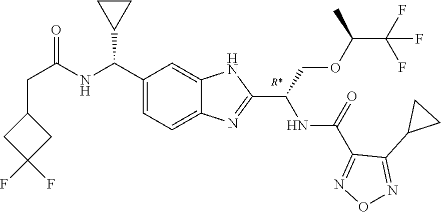 [SMILES: C[C@H](OC[C@H](NC(=O)c1nonc1C1CC1)c1nc2ccc([C@H](NC(=O)CC3CC(F)(F)C3)C3CC3)cc2[nH]1)C(F)(F)F]

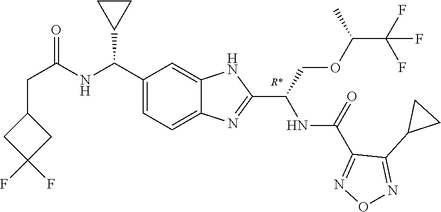 [SMILES: C[C@@H](OC[C@H](NC(=O)c1nonc1C1CC1)c1nc2ccc([C@H](NC(=O)CC3CC(F)(F)C3)C3CC3)cc2[nH]1)C(F)(F)F]

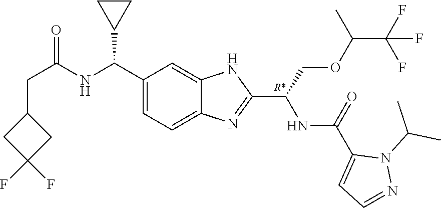 [SMILES: CC(C)n1nccc1C(=O)N[C@@H](COC(C)C(F)(F)F)c1nc2ccc([C@H](NC(=O)CC3CC(F)(F)C3)C3CC3)cc2[nH]1]

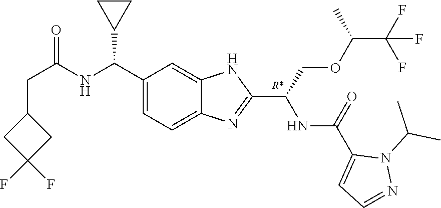 [SMILES: CC(C)n1nccc1C(=O)N[C@@H](CO[C@H](C)C(F)(F)F)c1nc2ccc([C@H](NC(=O)CC3CC(F)(F)C3)C3CC3)cc2[nH]1]

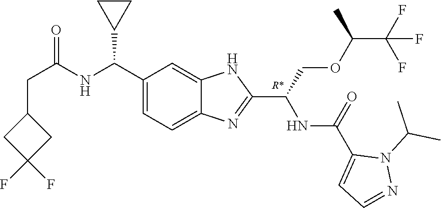 [SMILES: CC(C)n1nccc1C(=O)N[C@@H](CO[C@@H](C)C(F)(F)F)c1nc2ccc([C@H](NC(=O)CC3CC(F)(F)C3)C3CC3)cc2[nH]1]

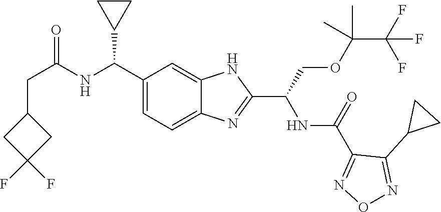 [SMILES: CC(C)(OC[C@H](NC(=O)c1nonc1C1CC1)c1nc2ccc([C@H](NC(=O)CC3CC(F)(F)C3)C3CC3)cc2[nH]1)C(F)(F)F]

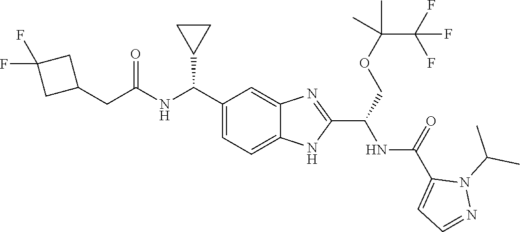 [SMILES: CC(C)n1nccc1C(=O)N[C@@H](COC(C)(C)C(F)(F)F)c1nc2cc([C@H](NC(=O)CC3CC(F)(F)C3)C3CC3)ccc2[nH]1]

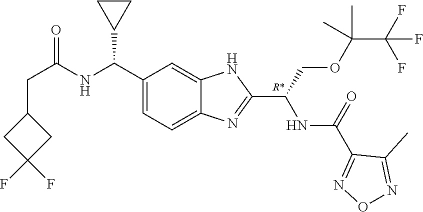 [SMILES: Cc1nonc1C(=O)N[C@@H](COC(C)(C)C(F)(F)F)c1nc2ccc([C@H](NC(=O)CC3CC(F)(F)C3)C3CC3)cc2[nH]1]

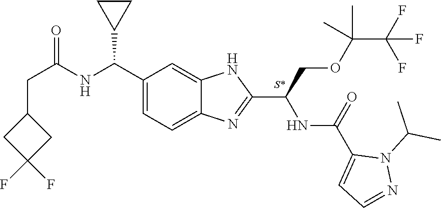 [SMILES: CC(C)n1nccc1C(=O)N[C@H](COC(C)(C)C(F)(F)F)c1nc2ccc([C@H](NC(=O)CC3CC(F)(F)C3)C3CC3)cc2[nH]1]

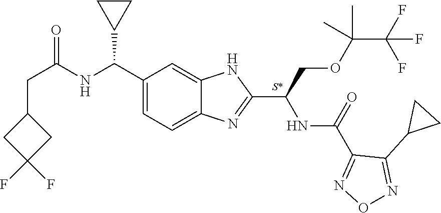 [SMILES: CC(C)(OC[C@@H](NC(=O)c1nonc1C1CC1)c1nc2ccc([C@H](NC(=O)CC3CC(F)(F)C3)C3CC3)cc2[nH]1)C(F)(F)F]

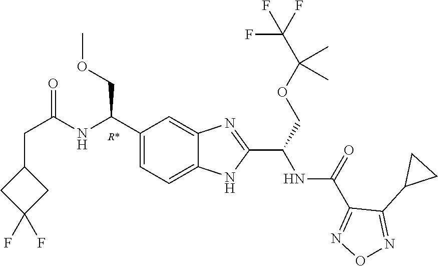 [SMILES: COC[C@H](NC(=O)CC1CC(F)(F)C1)c1ccc2[nH]c([C@H](COC(C)(C)C(F)(F)F)NC(=O)c3nonc3C3CC3)nc2c1]

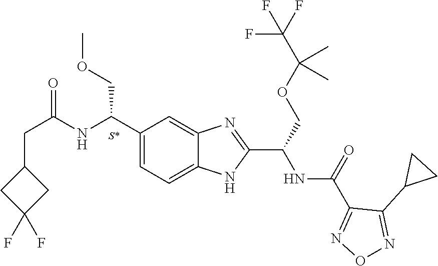 [SMILES: COC[C@@H](NC(=O)CC1CC(F)(F)C1)c1ccc2[nH]c([C@H](COC(C)(C)C(F)(F)F)NC(=O)c3nonc3C3CC3)nc2c1]

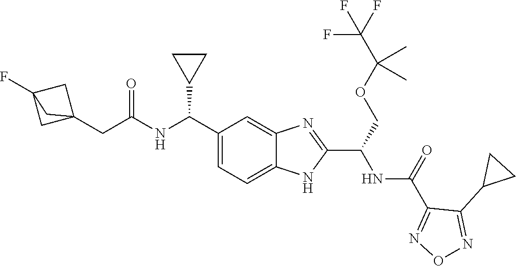 [SMILES: CC(C)(OC[C@H](NC(=O)c1nonc1C1CC1)c1nc2cc([C@H](NC(=O)CC34CC(F)(C3)C4)C3CC3)ccc2[nH]1)C(F)(F)F]

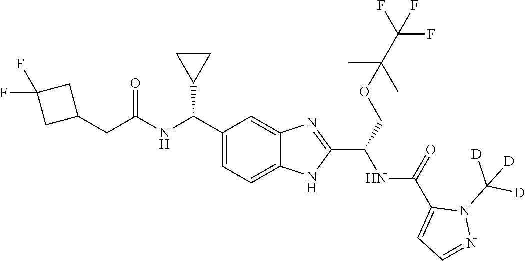 [SMILES: [2H]C([2H])([2H])n1nccc1C(=O)N[C@@H](COC(C)(C)C(F)(F)F)c1nc2cc([C@H](NC(=O)CC3CC(F)(F)C3)C3CC3)ccc2[nH]1]

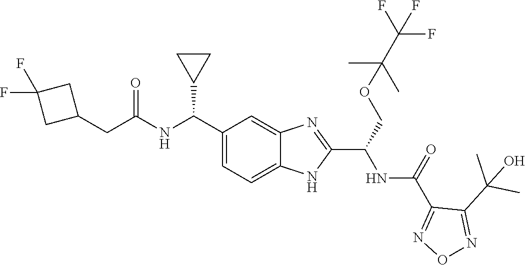 [SMILES: CC(C)(O)c1nonc1C(=O)N[C@@H](COC(C)(C)C(F)(F)F)c1nc2cc([C@H](NC(=O)CC3CC(F)(F)C3)C3CC3)ccc2[nH]1]

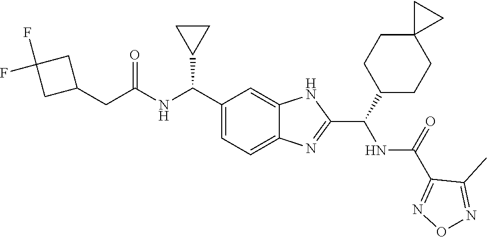 [SMILES: Cc1nonc1C(=O)N[C@H](c1nc2ccc([C@H](NC(=O)CC3CC(F)(F)C3)C3CC3)cc2[nH]1)C1CCC2(CC1)CC2]

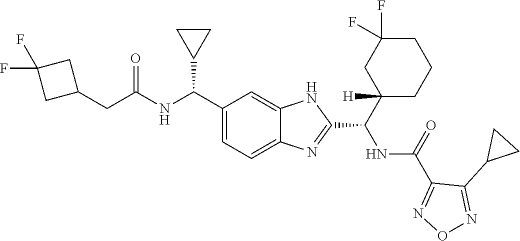 [SMILES: O=C(CC1CC(F)(F)C1)N[C@@H](c1ccc2nc([C@@H](NC(=O)c3nonc3C3CC3)[C@H]3CCCC(F)(F)C3)[nH]c2c1)C1CC1]

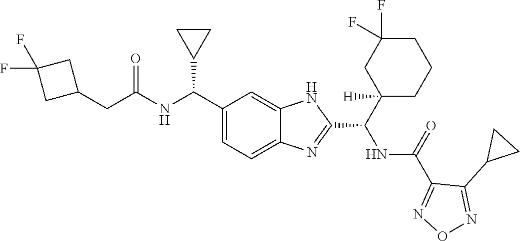 [SMILES: O=C(CC1CC(F)(F)C1)N[C@@H](c1ccc2nc([C@@H](NC(=O)c3nonc3C3CC3)[C@@H]3CCCC(F)(F)C3)[nH]c2c1)C1CC1]

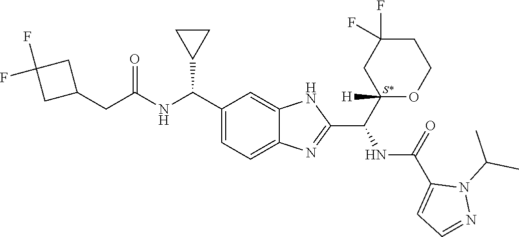 [SMILES: CC(C)n1nccc1C(=O)N[C@H](c1nc2ccc([C@H](NC(=O)CC3CC(F)(F)C3)C3CC3)cc2[nH]1)[C@@H]1CC(F)(F)CCO1]

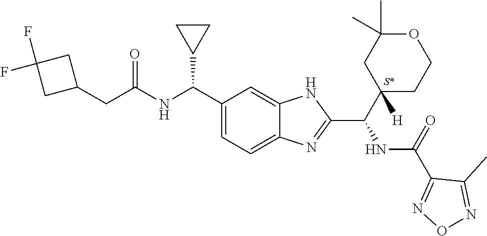 [SMILES: Cc1nonc1C(=O)N[C@H](c1nc2ccc([C@H](NC(=O)CC3CC(F)(F)C3)C3CC3)cc2[nH]1)[C@H]1CCOC(C)(C)C1]

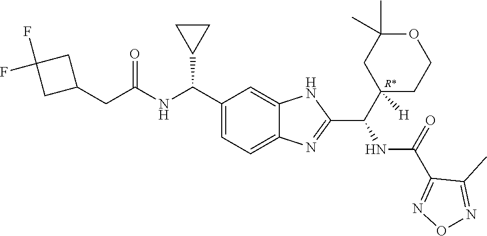 [SMILES: Cc1nonc1C(=O)N[C@H](c1nc2ccc([C@H](NC(=O)CC3CC(F)(F)C3)C3CC3)cc2[nH]1)[C@@H]1CCOC(C)(C)C1]

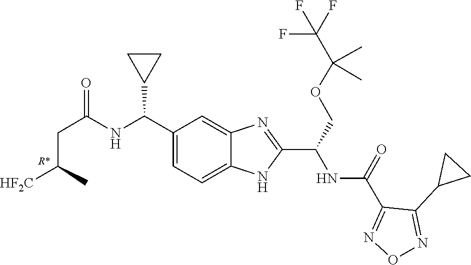

TABLE 1H

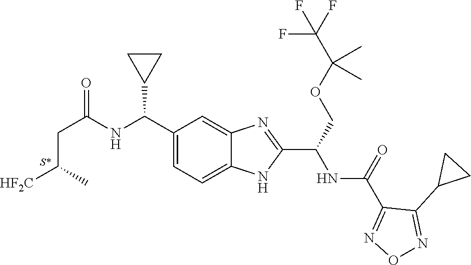

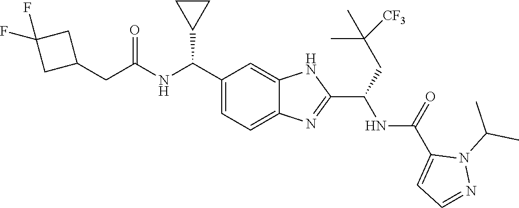 [SMILES: CC(C)n1nccc1C(=O)N[C@@H](CC(C)(C)C(F)(F)F)c1nc2ccc([C@H](NC(=O)CC3CC(F)(F)C3)C3CC3)cc2[nH]1]

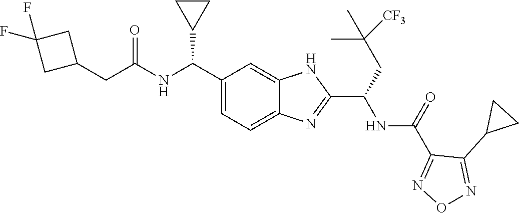 [SMILES: CC(C)(C[C@H](NC(=O)c1nonc1C1CC1)c1nc2ccc([C@H](NC(=O)CC3CC(F)(F)C3)C3CC3)cc2[nH]1)C(F)(F)F]

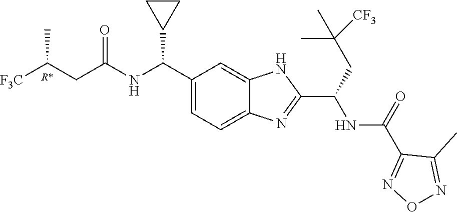 [SMILES: Cc1nonc1C(=O)N[C@@H](CC(C)(C)C(F)(F)F)c1nc2ccc([C@H](NC(=O)C[C@@H](C)C(F)(F)F)C3CC3)cc2[nH]1]

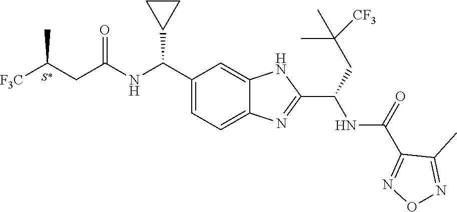 [SMILES: Cc1nonc1C(=O)N[C@@H](CC(C)(C)C(F)(F)F)c1nc2ccc([C@H](NC(=O)C[C@H](C)C(F)(F)F)C3CC3)cc2[nH]1]

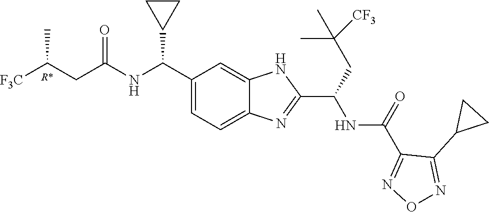 [SMILES: C[C@H](CC(=O)N[C@@H](c1ccc2nc([C@H](CC(C)(C)C(F)(F)F)NC(=O)c3nonc3C3CC3)[nH]c2c1)C1CC1)C(F)(F)F]

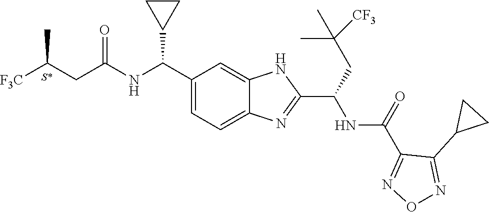 [SMILES: C[C@@H](CC(=O)N[C@@H](c1ccc2nc([C@H](CC(C)(C)C(F)(F)F)NC(=O)c3nonc3C3CC3)[nH]c2c1)C1CC1)C(F)(F)F]

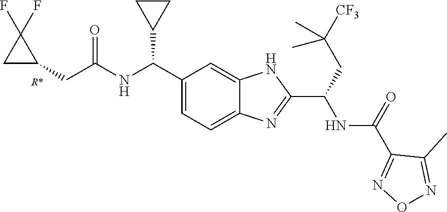 [SMILES: Cc1nonc1C(=O)N[C@@H](CC(C)(C)C(F)(F)F)c1nc2ccc([C@H](NC(=O)C[C@@H]3CC3(F)F)C3CC3)cc2[nH]1]

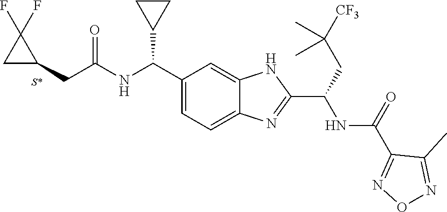 [SMILES: Cc1nonc1C(=O)N[C@@H](CC(C)(C)C(F)(F)F)c1nc2ccc([C@H](NC(=O)C[C@H]3CC3(F)F)C3CC3)cc2[nH]1]

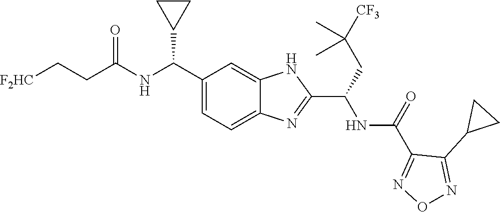 [SMILES: CC(C)(C[C@H](NC(=O)c1nonc1C1CC1)c1nc2ccc([C@H](NC(=O)CCC(F)F)C3CC3)cc2[nH]1)C(F)(F)F]

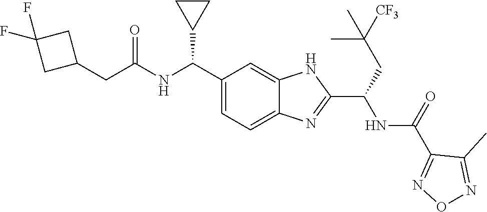 [SMILES: Cc1nonc1C(=O)N[C@@H](CC(C)(C)C(F)(F)F)c1nc2ccc([C@H](NC(=O)CC3CC(F)(F)C3)C3CC3)cc2[nH]1]

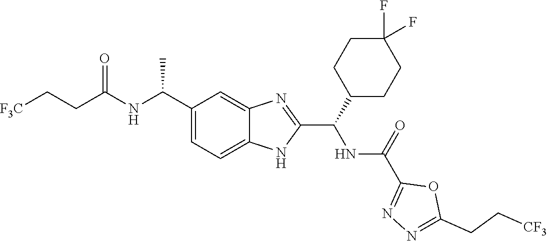 [SMILES: C[C@@H](NC(=O)CCC(F)(F)F)c1ccc2[nH]c([C@@H](NC(=O)c3nnc(CCC(F)(F)F)o3)C3CCC(F)(F)CC3)nc2c1]

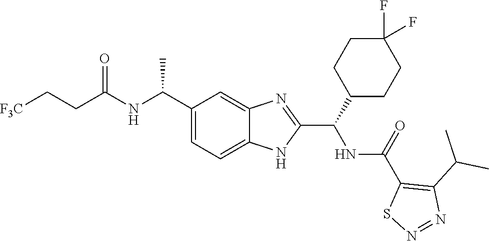 [SMILES: CC(C)c1nnsc1C(=O)N[C@H](c1nc2cc([C@@H](C)NC(=O)CCC(F)(F)F)ccc2[nH]1)C1CCC(F)(F)CC1]

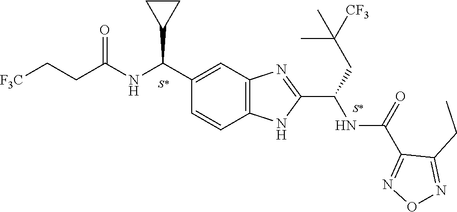 [SMILES: CCc1nonc1C(=O)N[C@@H](CC(C)(C)C(F)(F)F)c1nc2cc([C@@H](NC(=O)CCC(F)(F)F)C3CC3)ccc2[nH]1]

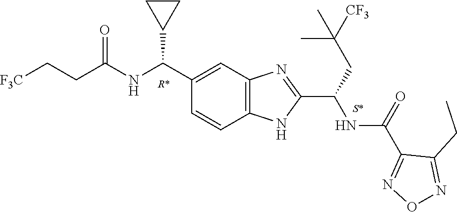 [SMILES: CCc1nonc1C(=O)N[C@@H](CC(C)(C)C(F)(F)F)c1nc2cc([C@H](NC(=O)CCC(F)(F)F)C3CC3)ccc2[nH]1]

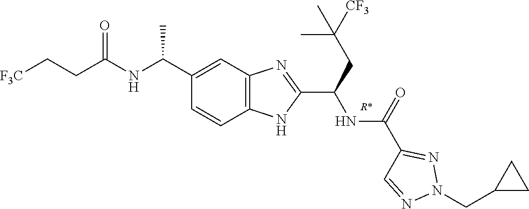 [SMILES: C[C@@H](NC(=O)CCC(F)(F)F)c1ccc2[nH]c([C@@H](CC(C)(C)C(F)(F)F)NC(=O)c3cnn(CC4CC4)n3)nc2c1]

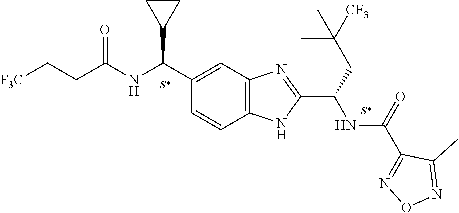 [SMILES: Cc1nonc1C(=O)N[C@@H](CC(C)(C)C(F)(F)F)c1nc2cc([C@@H](NC(=O)CCC(F)(F)F)C3CC3)ccc2[nH]1]

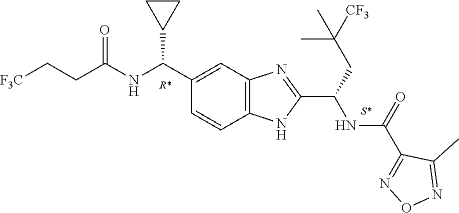 [SMILES: Cc1nonc1C(=O)N[C@@H](CC(C)(C)C(F)(F)F)c1nc2cc([C@H](NC(=O)CCC(F)(F)F)C3CC3)ccc2[nH]1]

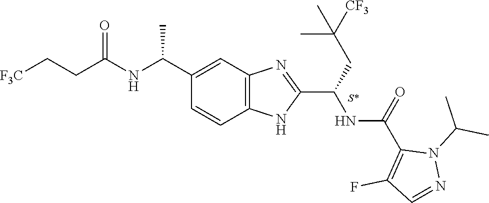 [SMILES: CC(C)n1ncc(F)c1C(=O)N[C@@H](CC(C)(C)C(F)(F)F)c1nc2cc([C@@H](C)NC(=O)CCC(F)(F)F)ccc2[nH]1]

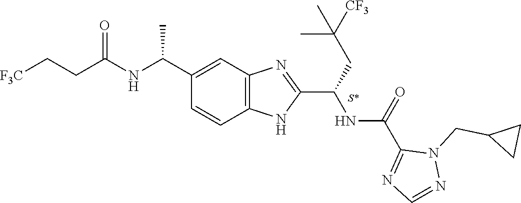 [SMILES: C[C@@H](NC(=O)CCC(F)(F)F)c1ccc2[nH]c([C@H](CC(C)(C)C(F)(F)F)NC(=O)c3ncnn3CC3CC3)nc2c1]

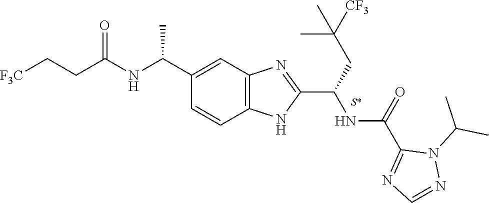 [SMILES: CC(C)n1ncnc1C(=O)N[C@@H](CC(C)(C)C(F)(F)F)c1nc2cc([C@@H](C)NC(=O)CCC(F)(F)F)ccc2[nH]1]

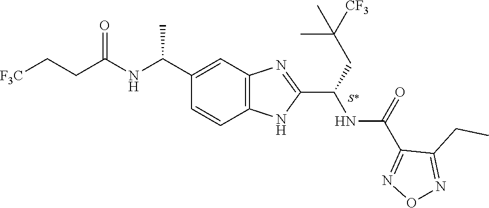 [SMILES: CCc1nonc1C(=O)N[C@@H](CC(C)(C)C(F)(F)F)c1nc2cc([C@@H](C)NC(=O)CCC(F)(F)F)ccc2[nH]1]

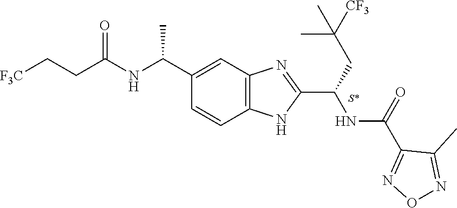 [SMILES: Cc1nonc1C(=O)N[C@@H](CC(C)(C)C(F)(F)F)c1nc2cc([C@@H](C)NC(=O)CCC(F)(F)F)ccc2[nH]1]

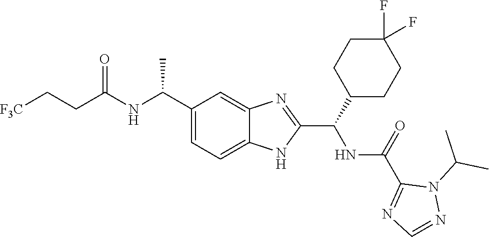 [SMILES: CC(C)n1ncnc1C(=O)N[C@H](c1nc2cc([C@@H](C)NC(=O)CCC(F)(F)F)ccc2[nH]1)C1CCC(F)(F)CC1]

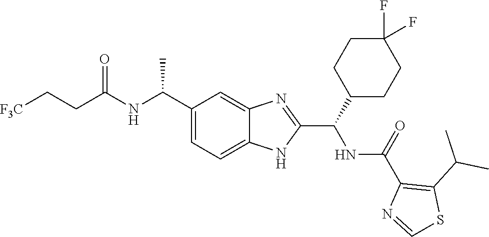 [SMILES: CC(C)c1scnc1C(=O)N[C@H](c1nc2cc([C@@H](C)NC(=O)CCC(F)(F)F)ccc2[nH]1)C1CCC(F)(F)CC1]

TABLE 1I

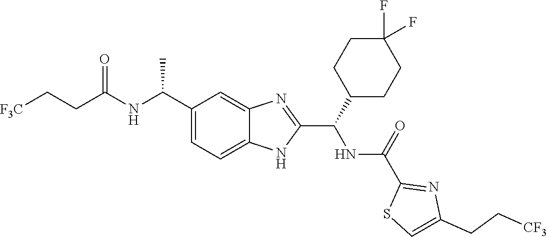 [SMILES: C[C@@H](NC(=O)CCC(F)(F)F)c1ccc2[nH]c([C@@H](NC(=O)c3nc(CCC(F)(F)F)cs3)C3CCC(F)(F)CC3)nc2c1]

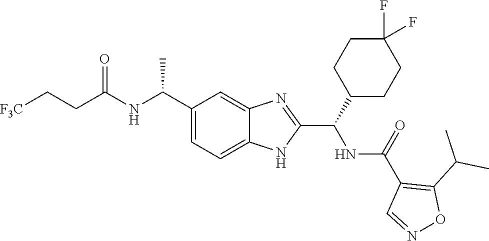 [SMILES: CC(C)c1oncc1C(=O)N[C@H](c1nc2cc([C@@H](C)NC(=O)CCC(F)(F)F)ccc2[nH]1)C1CCC(F)(F)CC1]

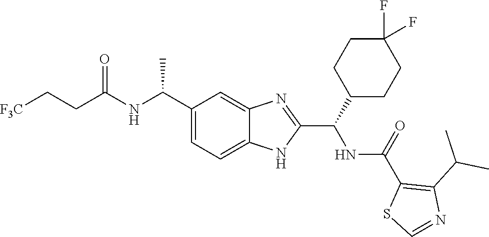 [SMILES: CC(C)c1ncsc1C(=O)N[C@H](c1nc2cc([C@@H](C)NC(=O)CCC(F)(F)F)ccc2[nH]1)C1CCC(F)(F)CC1]

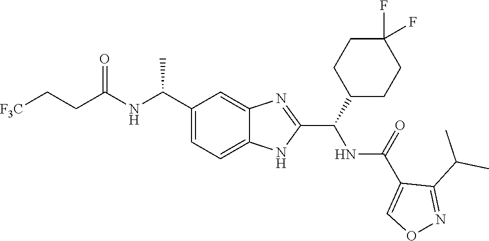 [SMILES: CC(C)c1nocc1C(=O)N[C@H](c1nc2cc([C@@H](C)NC(=O)CCC(F)(F)F)ccc2[nH]1)C1CCC(F)(F)CC1]

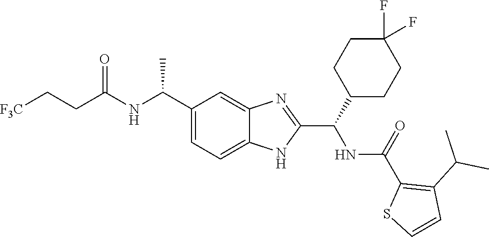 [SMILES: CC(C)c1ccsc1C(=O)N[C@H](c1nc2cc([C@@H](C)NC(=O)CCC(F)(F)F)ccc2[nH]1)C1CCC(F)(F)CC1]

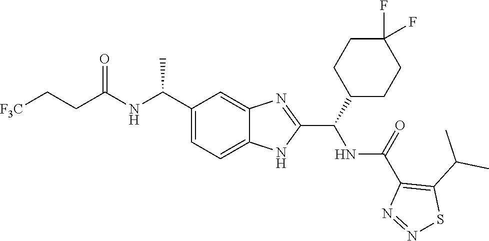 [SMILES: CC(C)c1snnc1C(=O)N[C@H](c1nc2cc([C@@H](C)NC(=O)CCC(F)(F)F)ccc2[nH]1)C1CCC(F)(F)CC1]

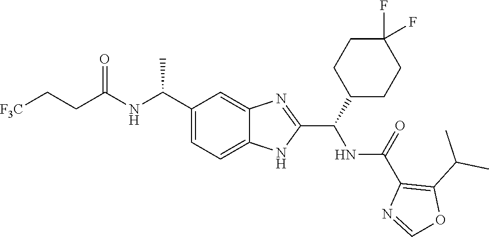 [SMILES: CC(C)c1ocnc1C(=O)N[C@H](c1nc2cc([C@@H](C)NC(=O)CCC(F)(F)F)ccc2[nH]1)C1CCC(F)(F)CC1]

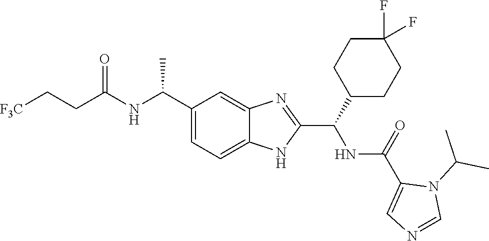 [SMILES: CC(C)n1cncc1C(=O)N[C@H](c1nc2cc([C@@H](C)NC(=O)CCC(F)(F)F)ccc2[nH]1)C1CCC(F)(F)CC1]

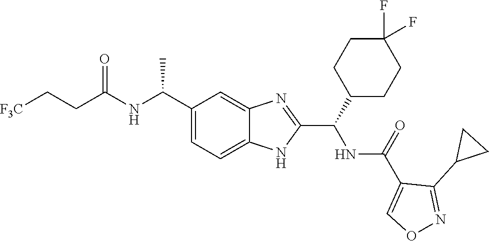 [SMILES: C[C@@H](NC(=O)CCC(F)(F)F)c1ccc2[nH]c([C@@H](NC(=O)c3conc3C3CC3)C3CCC(F)(F)CC3)nc2c1]

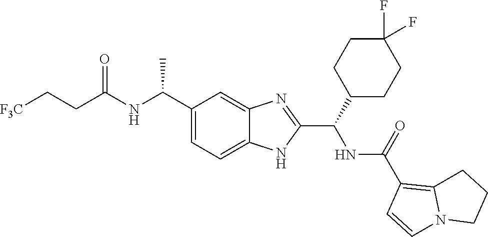 [SMILES: C[C@@H](NC(=O)CCC(F)(F)F)c1ccc2[nH]c([C@@H](NC(=O)c3ccn4c3CCC4)C3CCC(F)(F)CC3)nc2c1]

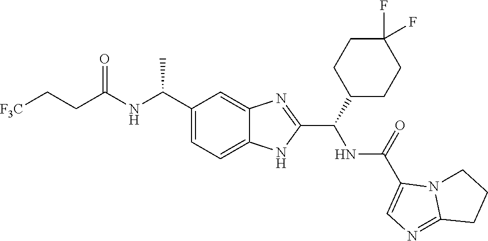 [SMILES: C[C@@H](NC(=O)CCC(F)(F)F)c1ccc2[nH]c([C@@H](NC(=O)c3cnc4n3CCC4)C3CCC(F)(F)CC3)nc2c1]

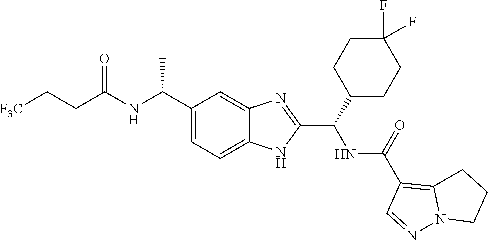 [SMILES: C[C@@H](NC(=O)CCC(F)(F)F)c1ccc2[nH]c([C@@H](NC(=O)c3cnn4c3CCC4)C3CCC(F)(F)CC3)nc2c1]

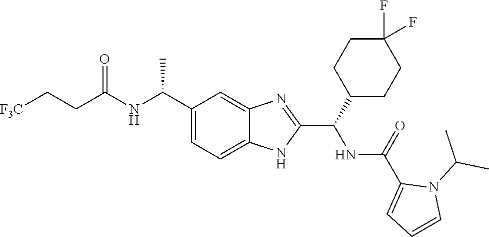 [SMILES: CC(C)n1cccc1C(=O)N[C@H](c1nc2cc([C@@H](C)NC(=O)CCC(F)(F)F)ccc2[nH]1)C1CCC(F)(F)CC1]

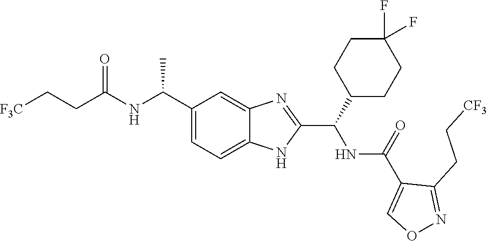 [SMILES: C[C@@H](NC(=O)CCC(F)(F)F)c1ccc2[nH]c([C@@H](NC(=O)c3conc3CCC(F)(F)F)C3CCC(F)(F)CC3)nc2c1]

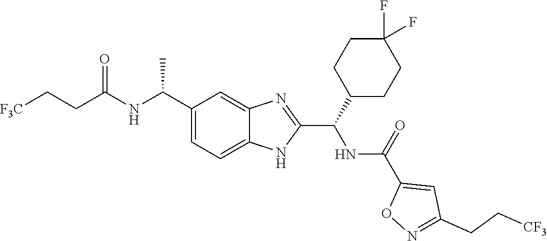 [SMILES: C[C@@H](NC(=O)CCC(F)(F)F)c1ccc2[nH]c([C@@H](NC(=O)c3cc(CCC(F)(F)F)no3)C3CCC(F)(F)CC3)nc2c1]

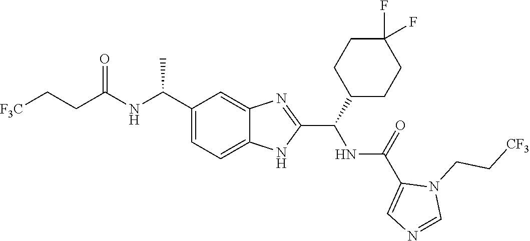 [SMILES: C[C@@H](NC(=O)CCC(F)(F)F)c1ccc2[nH]c([C@@H](NC(=O)c3cncn3CCC(F)(F)F)C3CCC(F)(F)CC3)nc2c1]

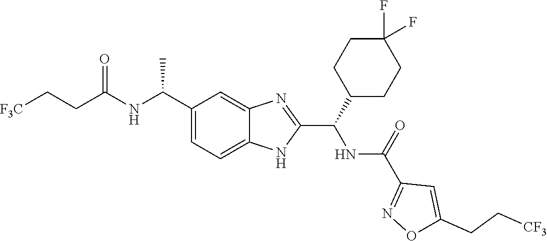 [SMILES: C[C@@H](NC(=O)CCC(F)(F)F)c1ccc2[nH]c([C@@H](NC(=O)c3cc(CCC(F)(F)F)on3)C3CCC(F)(F)CC3)nc2c1]

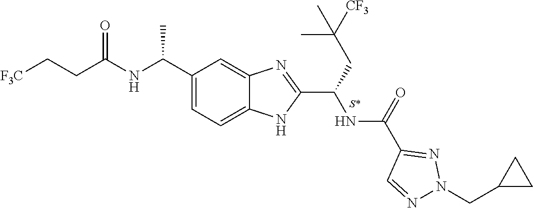 [SMILES: C[C@@H](NC(=O)CCC(F)(F)F)c1ccc2[nH]c([C@H](CC(C)(C)C(F)(F)F)NC(=O)c3cnn(CC4CC4)n3)nc2c1]

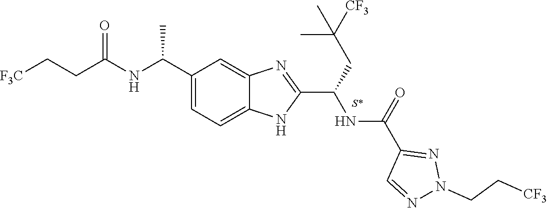 [SMILES: C[C@@H](NC(=O)CCC(F)(F)F)c1ccc2[nH]c([C@H](CC(C)(C)C(F)(F)F)NC(=O)c3cnn(CCC(F)(F)F)n3)nc2c1]

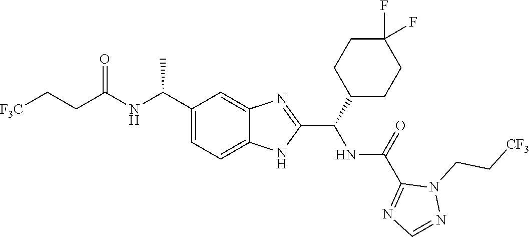 [SMILES: C[C@@H](NC(=O)CCC(F)(F)F)c1ccc2[nH]c([C@@H](NC(=O)c3ncnn3CCC(F)(F)F)C3CCC(F)(F)CC3)nc2c1]

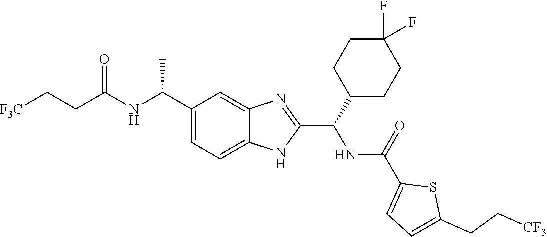 [SMILES: C[C@@H](NC(=O)CCC(F)(F)F)c1ccc2[nH]c([C@@H](NC(=O)c3ccc(CCC(F)(F)F)s3)C3CCC(F)(F)CC3)nc2c1]

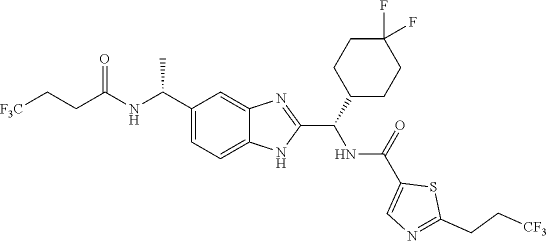 [SMILES: C[C@@H](NC(=O)CCC(F)(F)F)c1ccc2[nH]c([C@@H](NC(=O)c3cnc(CCC(F)(F)F)s3)C3CCC(F)(F)CC3)nc2c1]

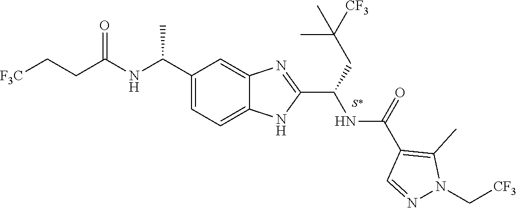 [SMILES: Cc1c(C(=O)N[C@@H](CC(C)(C)C(F)(F)F)c2nc3cc([C@@H](C)NC(=O)CCC(F)(F)F)ccc3[nH]2)cnn1CC(F)(F)F]

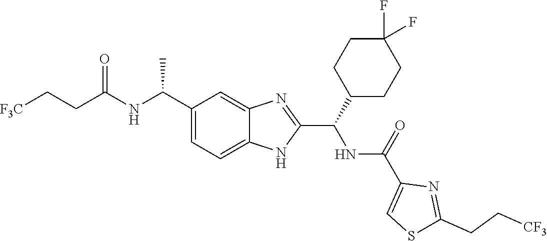 [SMILES: C[C@@H](NC(=O)CCC(F)(F)F)c1ccc2[nH]c([C@@H](NC(=O)c3csc(CCC(F)(F)F)n3)C3CCC(F)(F)CC3)nc2c1]

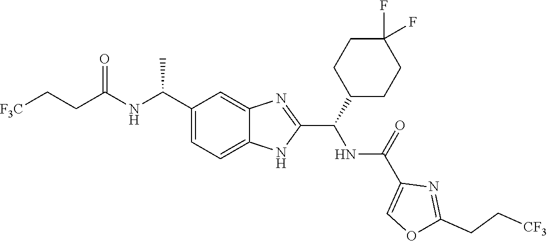 [SMILES: C[C@@H](NC(=O)CCC(F)(F)F)c1ccc2[nH]c([C@@H](NC(=O)c3coc(CCC(F)(F)F)n3)C3CCC(F)(F)CC3)nc2c1]

TABLE 1J

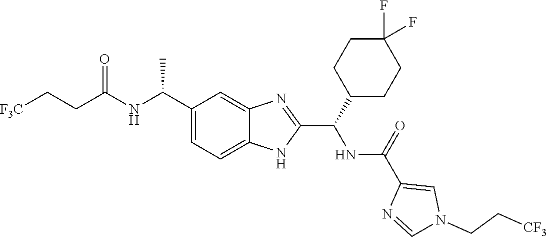 [SMILES: C[C@@H](NC(=O)CCC(F)(F)F)c1ccc2[nH]c([C@@H](NC(=O)c3cn(CCC(F)(F)F)cn3)C3CCC(F)(F)CC3)nc2c1]

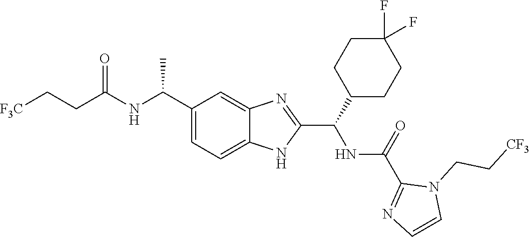 [SMILES: C[C@@H](NC(=O)CCC(F)(F)F)c1ccc2[nH]c([C@@H](NC(=O)c3nccn3CCC(F)(F)F)C3CCC(F)(F)CC3)nc2c1]

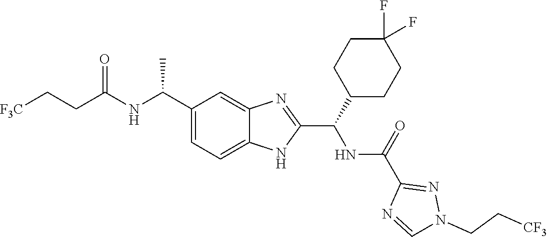 [SMILES: C[C@@H](NC(=O)CCC(F)(F)F)c1ccc2[nH]c([C@@H](NC(=O)c3ncn(CCC(F)(F)F)n3)C3CCC(F)(F)CC3)nc2c1]

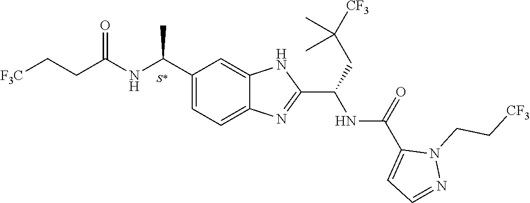 [SMILES: C[C@H](NC(=O)CCC(F)(F)F)c1ccc2nc([C@H](CC(C)(C)C(F)(F)F)NC(=O)c3ccnn3CCC(F)(F)F)[nH]c2c1]

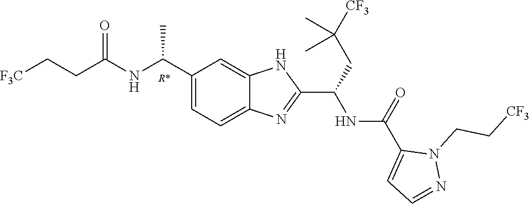 [SMILES: C[C@@H](NC(=O)CCC(F)(F)F)c1ccc2nc([C@H](CC(C)(C)C(F)(F)F)NC(=O)c3ccnn3CCC(F)(F)F)[nH]c2c1]

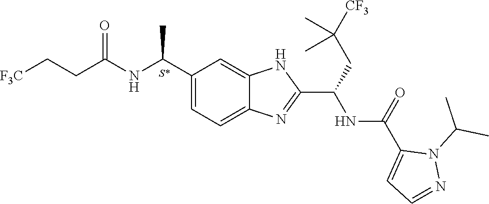 [SMILES: CC(C)n1nccc1C(=O)N[C@@H](CC(C)(C)C(F)(F)F)c1nc2ccc([C@H](C)NC(=O)CCC(F)(F)F)cc2[nH]1]

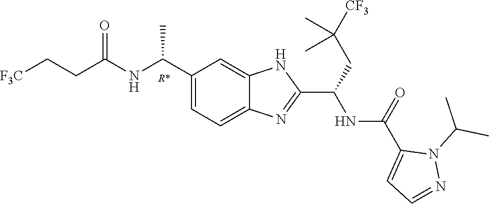 [SMILES: CC(C)n1nccc1C(=O)N[C@@H](CC(C)(C)C(F)(F)F)c1nc2ccc([C@@H](C)NC(=O)CCC(F)(F)F)cc2[nH]1]

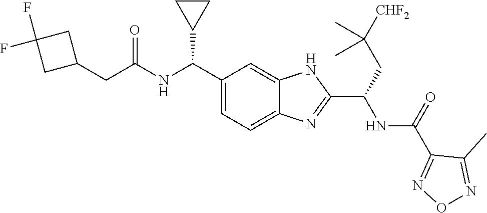 [SMILES: Cc1nonc1C(=O)N[C@@H](CC(C)(C)C(F)F)c1nc2ccc([C@H](NC(=O)CC3CC(F)(F)C3)C3CC3)cc2[nH]1]

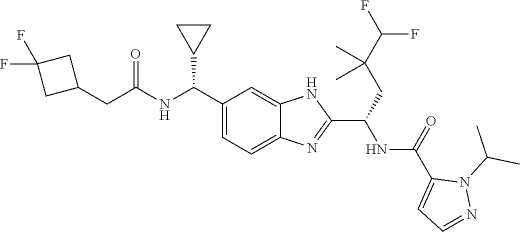 [SMILES: CC(C)n1nccc1C(=O)N[C@@H](CC(C)(C)C(F)F)c1nc2ccc([C@H](NC(=O)CC3CC(F)(F)C3)C3CC3)cc2[nH]1]

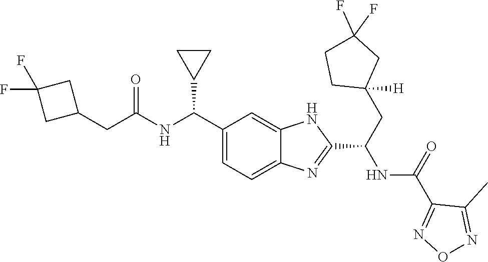 [SMILES: Cc1nonc1C(=O)N[C@@H](C[C@H]1CCC(F)(F)C1)c1nc2ccc([C@H](NC(=O)CC3CC(F)(F)C3)C3CC3)cc2[nH]1]

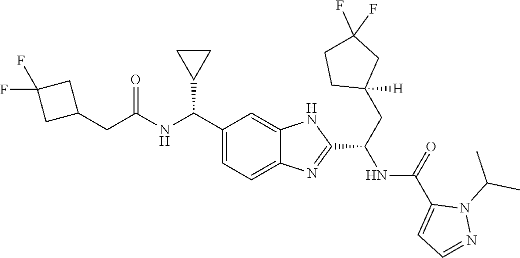 [SMILES: CC(C)n1nccc1C(=O)N[C@@H](C[C@H]1CCC(F)(F)C1)c1nc2ccc([C@H](NC(=O)CC3CC(F)(F)C3)C3CC3)cc2[nH]1]

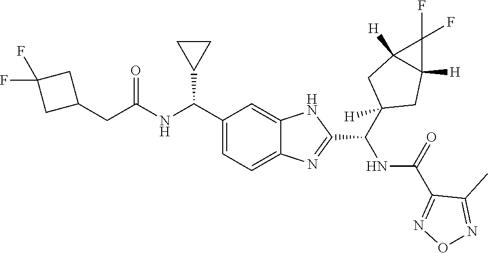 [SMILES: Cc1nonc1C(=O)N[C@H](c1nc2ccc([C@H](NC(=O)CC3CC(F)(F)C3)C3CC3)cc2[nH]1)[C@@H]1C[C@@H]2[C@H](C1)C2(F)F]

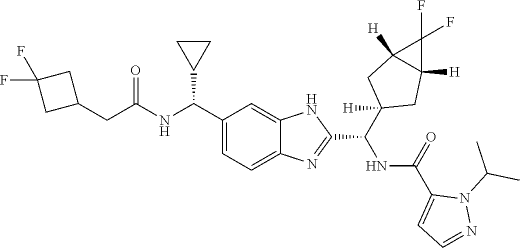 [SMILES: CC(C)n1nccc1C(=O)N[C@H](c1nc2ccc([C@H](NC(=O)CC3CC(F)(F)C3)C3CC3)cc2[nH]1)[C@@H]1C[C@@H]2[C@H](C1)C2(F)F]

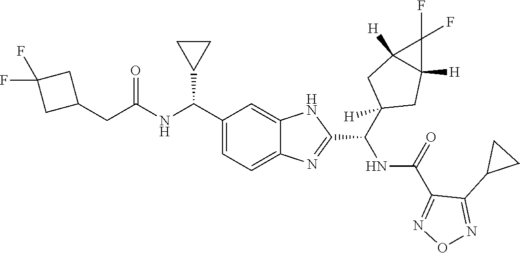 [SMILES: O=C(CC1CC(F)(F)C1)N[C@@H](c1ccc2nc([C@@H](NC(=O)c3nonc3C3CC3)[C@@H]3C[C@@H]4[C@H](C3)C4(F)F)[nH]c2c1)C1CC1]

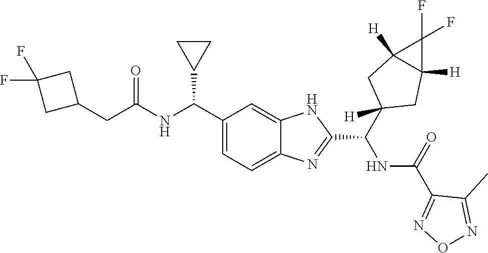 [SMILES: Cc1nonc1C(=O)N[C@H](c1nc2ccc([C@H](NC(=O)CC3CC(F)(F)C3)C3CC3)cc2[nH]1)[C@H]1C[C@@H]2[C@H](C1)C2(F)F]

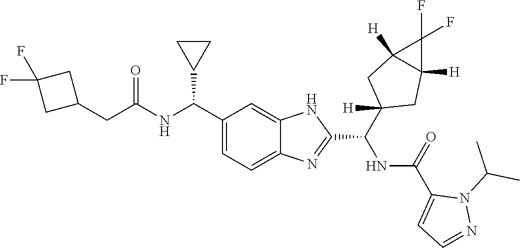 [SMILES: CC(C)n1nccc1C(=O)N[C@H](c1nc2ccc([C@H](NC(=O)CC3CC(F)(F)C3)C3CC3)cc2[nH]1)[C@H]1C[C@@H]2[C@H](C1)C2(F)F]

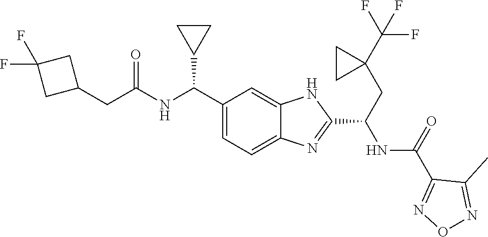 [SMILES: Cc1nonc1C(=O)N[C@@H](CC1(C(F)(F)F)CC1)c1nc2ccc([C@H](NC(=O)CC3CC(F)(F)C3)C3CC3)cc2[nH]1]

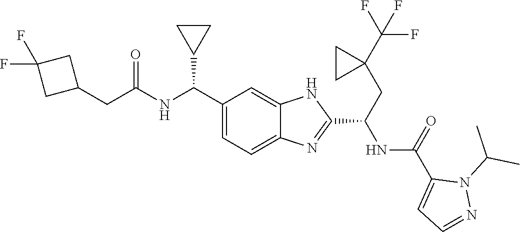 [SMILES: CC(C)n1nccc1C(=O)N[C@@H](CC1(C(F)(F)F)CC1)c1nc2ccc([C@H](NC(=O)CC3CC(F)(F)C3)C3CC3)cc2[nH]1]

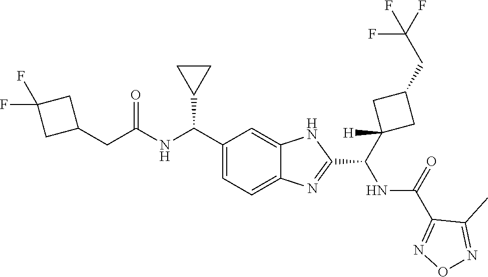 [SMILES: Cc1nonc1C(=O)N[C@H](c1nc2ccc([C@H](NC(=O)CC3CC(F)(F)C3)C3CC3)cc2[nH]1)[C@H]1C[C@@H](CC(F)(F)F)C1]

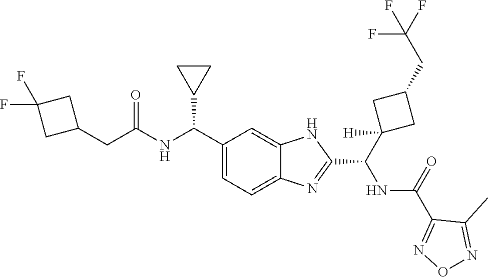 [SMILES: Cc1nonc1C(=O)N[C@H](c1nc2ccc([C@H](NC(=O)CC3CC(F)(F)C3)C3CC3)cc2[nH]1)[C@H]1C[C@H](CC(F)(F)F)C1]

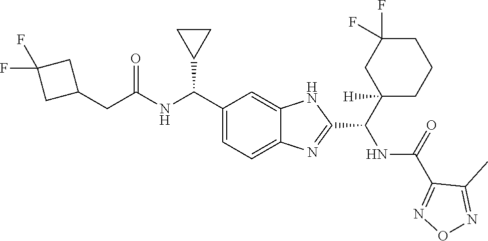 [SMILES: Cc1nonc1C(=O)N[C@H](c1nc2ccc([C@H](NC(=O)CC3CC(F)(F)C3)C3CC3)cc2[nH]1)[C@@H]1CCCC(F)(F)C1]

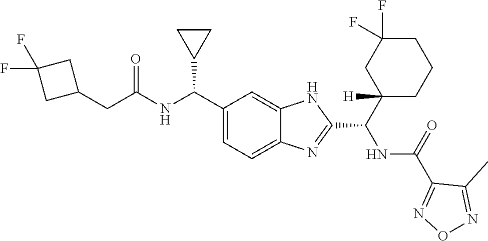 [SMILES: Cc1nonc1C(=O)N[C@H](c1nc2ccc([C@H](NC(=O)CC3CC(F)(F)C3)C3CC3)cc2[nH]1)[C@H]1CCCC(F)(F)C1]

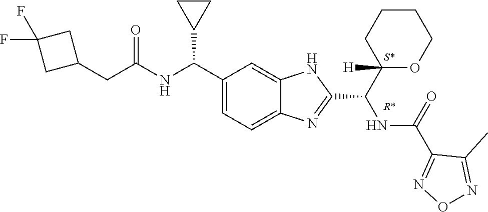 [SMILES: Cc1nonc1C(=O)N[C@H](c1nc2ccc([C@H](NC(=O)CC3CC(F)(F)C3)C3CC3)cc2[nH]1)[C@@H]1CCCCO1]

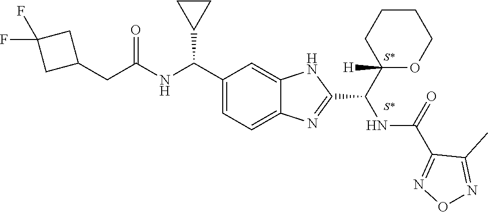 [SMILES: Cc1nonc1C(=O)N[C@H](c1nc2ccc([C@H](NC(=O)CC3CC(F)(F)C3)C3CC3)cc2[nH]1)[C@@H]1CCCCO1]

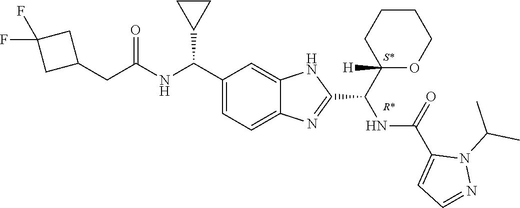 [SMILES: CC(C)n1nccc1C(=O)N[C@H](c1nc2ccc([C@H](NC(=O)CC3CC(F)(F)C3)C3CC3)cc2[nH]1)[C@@H]1CCCCO1]

TABLE 1K

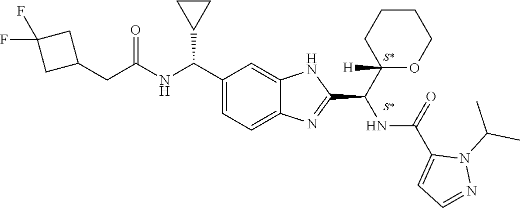 [SMILES: CC(C)n1nccc1C(=O)N[C@@H](c1nc2ccc([C@H](NC(=O)CC3CC(F)(F)C3)C3CC3)cc2[nH]1)[C@@H]1CCCCO1]

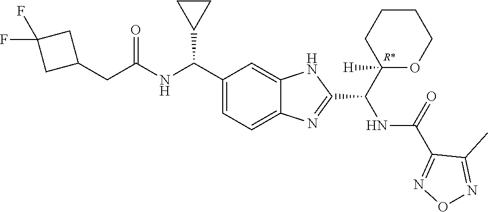 [SMILES: Cc1nonc1C(=O)N[C@H](c1nc2ccc([C@H](NC(=O)CC3CC(F)(F)C3)C3CC3)cc2[nH]1)[C@H]1CCCCO1]

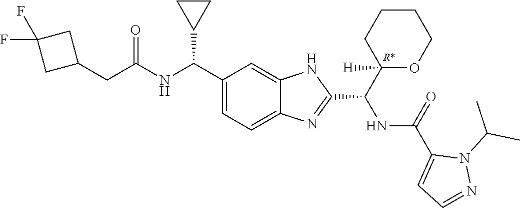 [SMILES: CC(C)n1nccc1C(=O)N[C@H](c1nc2ccc([C@H](NC(=O)CC3CC(F)(F)C3)C3CC3)cc2[nH]1)[C@H]1CCCCO1]

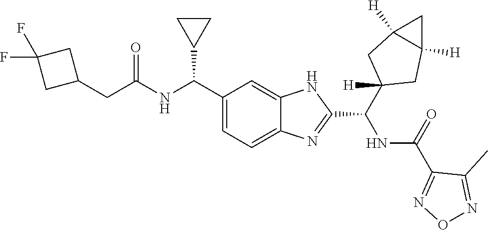 [SMILES: Cc1nonc1C(=O)N[C@H](c1nc2ccc([C@H](NC(=O)CC3CC(F)(F)C3)C3CC3)cc2[nH]1)[C@@H]1C[C@H]2C[C@H]2C1]

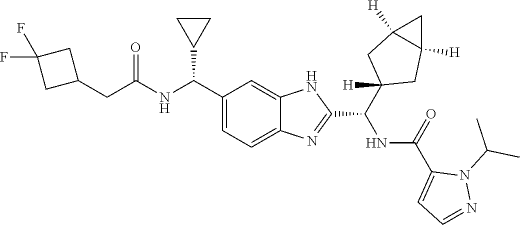 [SMILES: CC(C)n1nccc1C(=O)N[C@H](c1nc2ccc([C@H](NC(=O)CC3CC(F)(F)C3)C3CC3)cc2[nH]1)[C@@H]1C[C@H]2C[C@H]2C1]

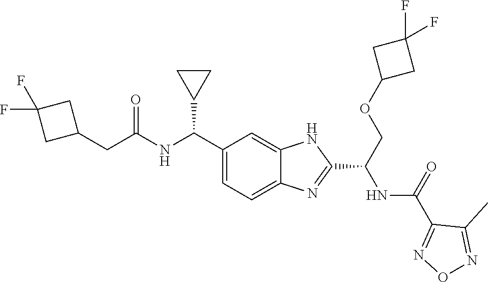 [SMILES: Cc1nonc1C(=O)N[C@@H](COC1CC(F)(F)C1)c1nc2ccc([C@H](NC(=O)CC3CC(F)(F)C3)C3CC3)cc2[nH]1]

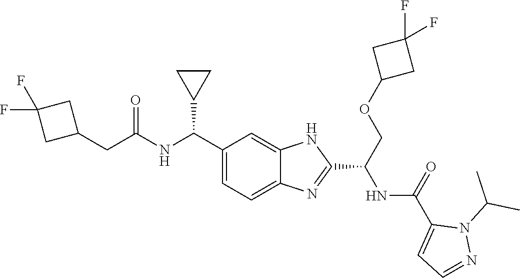 [SMILES: CC(C)n1nccc1C(=O)N[C@@H](COC1CC(F)(F)C1)c1nc2ccc([C@H](NC(=O)CC3CC(F)(F)C3)C3CC3)cc2[nH]1]

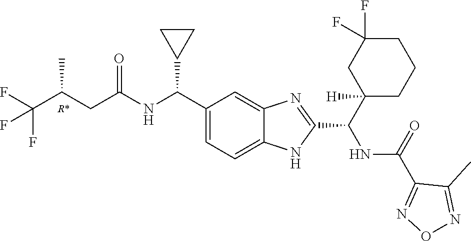 [SMILES: Cc1nonc1C(=O)N[C@H](c1nc2cc([C@H](NC(=O)C[C@@H](C)C(F)(F)F)C3CC3)ccc2[nH]1)[C@@H]1CCCC(F)(F)C1]

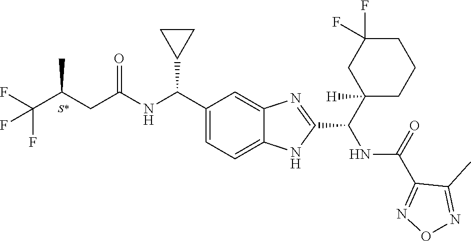 [SMILES: Cc1nonc1C(=O)N[C@H](c1nc2cc([C@H](NC(=O)C[C@H](C)C(F)(F)F)C3CC3)ccc2[nH]1)[C@@H]1CCCC(F)(F)C1]

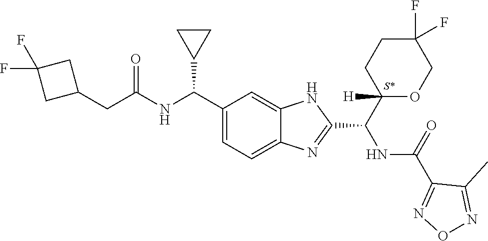 [SMILES: Cc1nonc1C(=O)N[C@H](c1nc2ccc([C@H](NC(=O)CC3CC(F)(F)C3)C3CC3)cc2[nH]1)[C@@H]1CCC(F)(F)CO1]

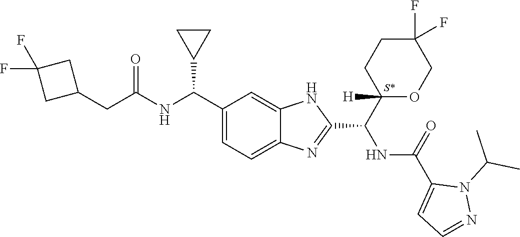 [SMILES: CC(C)n1nccc1C(=O)N[C@H](c1nc2ccc([C@H](NC(=O)CC3CC(F)(F)C3)C3CC3)cc2[nH]1)[C@@H]1CCC(F)(F)CO1]

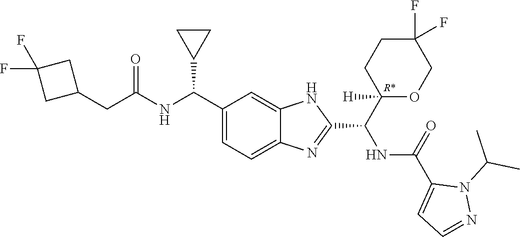 [SMILES: CC(C)n1nccc1C(=O)N[C@H](c1nc2ccc([C@H](NC(=O)CC3CC(F)(F)C3)C3CC3)cc2[nH]1)[C@H]1CCC(F)(F)CO1]

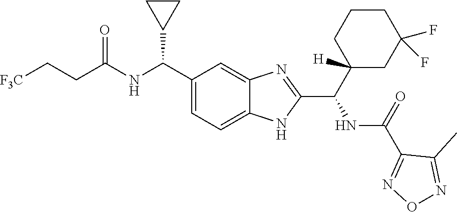 [SMILES: Cc1nonc1C(=O)N[C@H](c1nc2cc([C@H](NC(=O)CCC(F)(F)F)C3CC3)ccc2[nH]1)[C@@H]1CCCC(F)(F)C1]

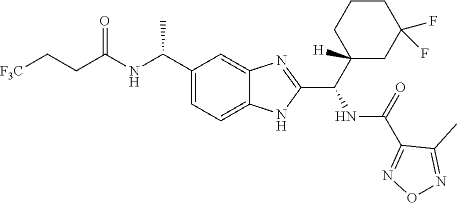 [SMILES: Cc1nonc1C(=O)N[C@H](c1nc2cc([C@@H](C)NC(=O)CCC(F)(F)F)ccc2[nH]1)[C@@H]1CCCC(F)(F)C1]

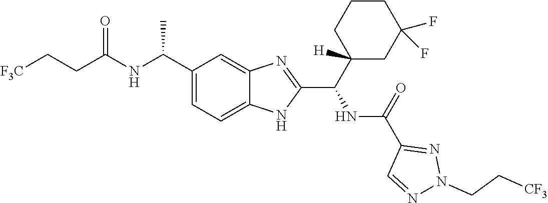 [SMILES: C[C@@H](NC(=O)CCC(F)(F)F)c1ccc2[nH]c([C@@H](NC(=O)c3cnn(CCC(F)(F)F)n3)[C@@H]3CCCC(F)(F)C3)nc2c1]

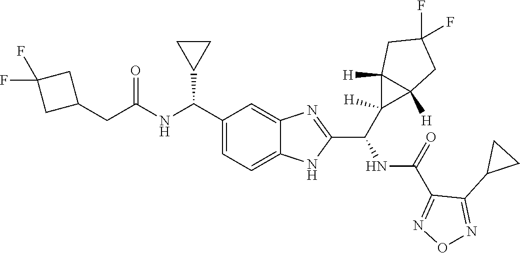 [SMILES: O=C(CC1CC(F)(F)C1)N[C@@H](c1ccc2[nH]c([C@@H](NC(=O)c3nonc3C3CC3)[C@H]3[C@@H]4CC(F)(F)C[C@@H]43)nc2c1)C1CC1]

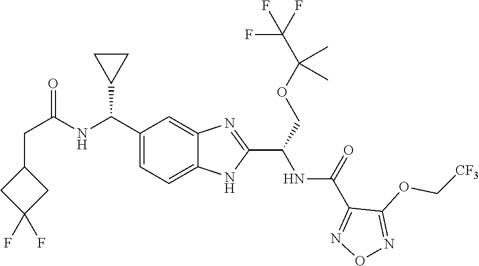 [SMILES: CC(C)(OC[C@H](NC(=O)c1nonc1OCC(F)(F)F)c1nc2cc([C@H](NC(=O)CC3CC(F)(F)C3)C3CC3)ccc2[nH]1)C(F)(F)F]

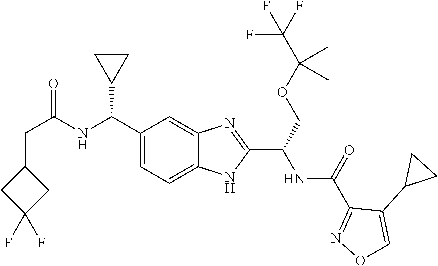 [SMILES: CC(C)(OC[C@H](NC(=O)c1nocc1C1CC1)c1nc2cc([C@H](NC(=O)CC3CC(F)(F)C3)C3CC3)ccc2[nH]1)C(F)(F)F]

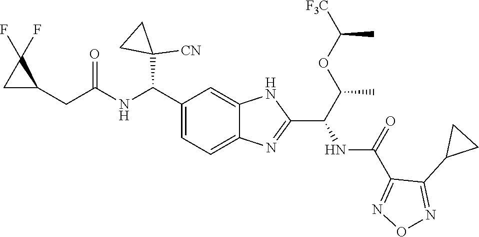 [SMILES: C[C@@H](O[C@H](C)C(F)(F)F)[C@H](NC(=O)c1nonc1C1CC1)c1nc2ccc([C@H](NC(=O)C[C@H]3CC3(F)F)C3(C#N)CC3)cc2[nH]1]

TABLE 1L

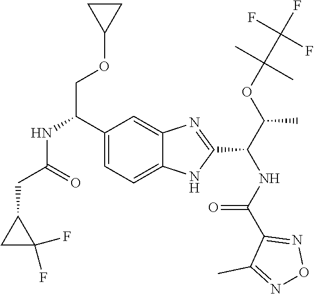 [SMILES: Cc1nonc1C(=O)N[C@H](c1nc2cc([C@@H](COC3CC3)NC(=O)C[C@H]3CC3(F)F)ccc2[nH]1)[C@@H](C)OC(C)(C)C(F)(F)F]

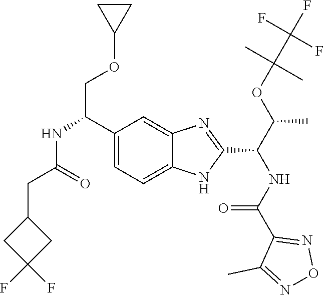 [SMILES: Cc1nonc1C(=O)N[C@H](c1nc2cc([C@@H](COC3CC3)NC(=O)CC3CC(F)(F)C3)ccc2[nH]1)[C@@H](C)OC(C)(C)C(F)(F)F]

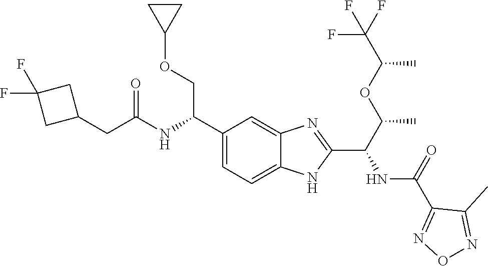 [SMILES: Cc1nonc1C(=O)N[C@H](c1nc2cc([C@@H](COC3CC3)NC(=O)CC3CC(F)(F)C3)ccc2[nH]1)[C@@H](C)O[C@@H](C)C(F)(F)F]

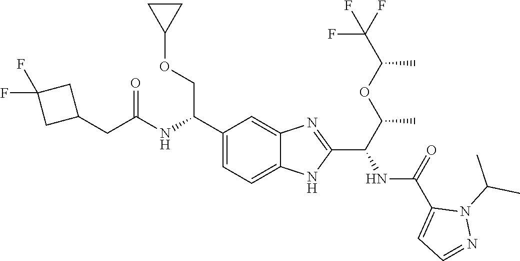 [SMILES: CC(C)n1nccc1C(=O)N[C@H](c1nc2cc([C@@H](COC3CC3)NC(=O)CC3CC(F)(F)C3)ccc2[nH]1)[C@@H](C)O[C@@H](C)C(F)(F)F]

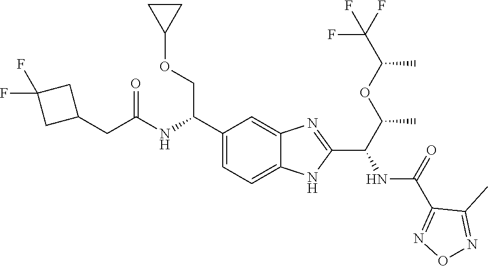 [SMILES: Cc1nonc1C(=O)N[C@H](c1nc2cc([C@@H](COC3CC3)NC(=O)CC3CC(F)(F)C3)ccc2[nH]1)[C@@H](C)O[C@@H](C)C(F)(F)F]

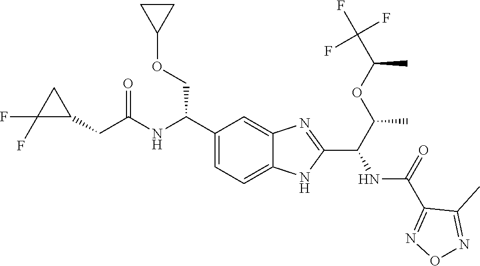

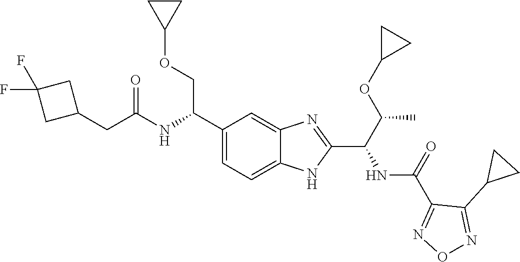 [SMILES: C[C@@H](OC1CC1)[C@H](NC(=O)c1nonc1C1CC1)c1nc2cc([C@@H](COC3CC3)NC(=O)CC3CC(F)(F)C3)ccc2[nH]1]

In some embodiments, disclosed herein is a compound of Formula (I), or a pharmaceutically acceptable salt thereof, selected from the group consisting of:

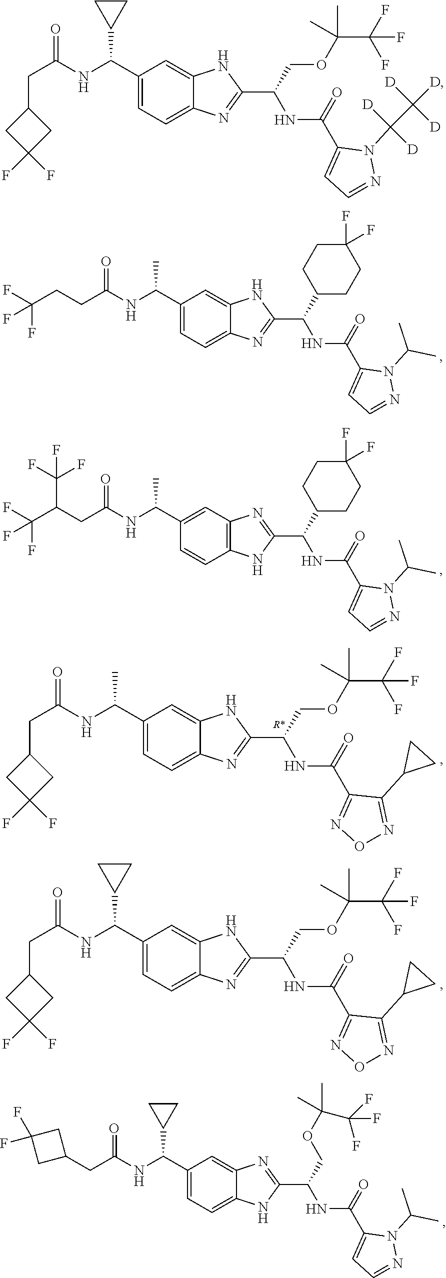

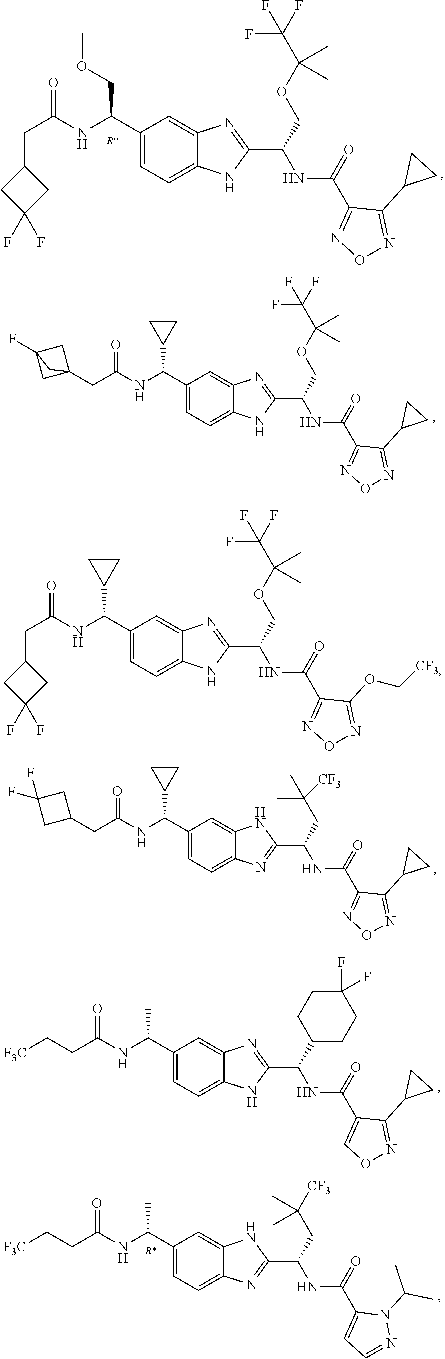

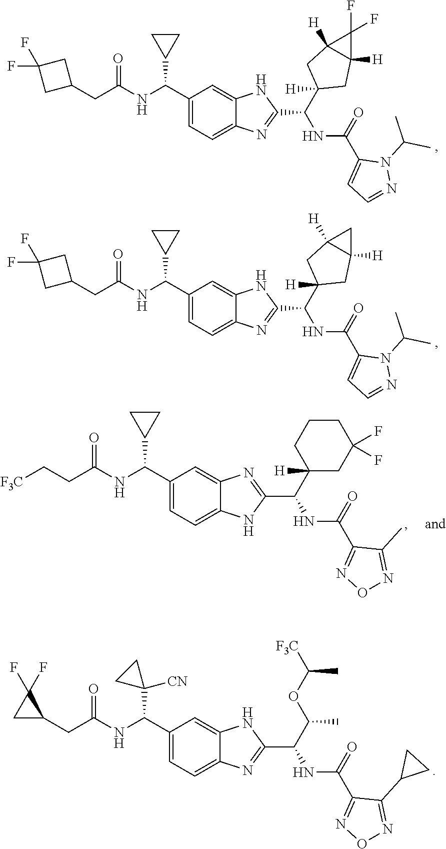

In some embodiments, disclosed herein is a compound of Formula (I) having the following structure:

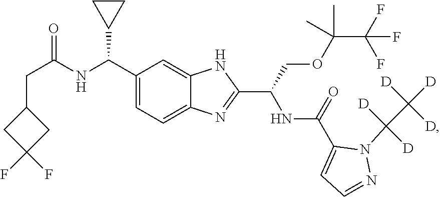

or a pharmaceutically acceptable salt thereof.

In some embodiments, disclosed herein is a compound of Formula (I) having the following structure:

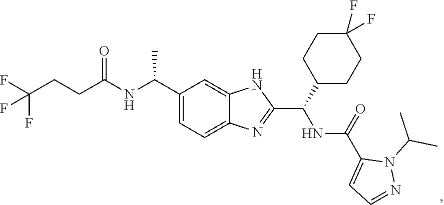 [SMILES: CC(C)n1nccc1C(=O)N[C@H](c1nc2ccc([C@@H](C)NC(=O)CCC(F)(F)F)cc2[nH]1)C1CCC(F)(F)CC1]

or a pharmaceutically acceptable salt thereof.

In some embodiments, disclosed herein is a compound of Formula (I) having the following structure:

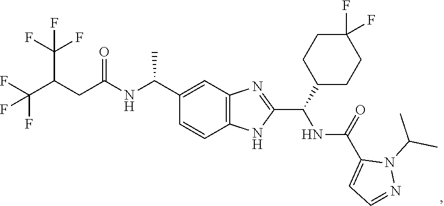 [SMILES: CC(C)n1nccc1C(=O)N[C@H](c1nc2cc([C@@H](C)NC(=O)CC(C(F)(F)F)C(F)(F)F)ccc2[nH]1)C1CCC(F)(F)CC1]

or a pharmaceutically acceptable salt thereof.

In some embodiments, disclosed herein is a compound of Formula (I) having the following structure:

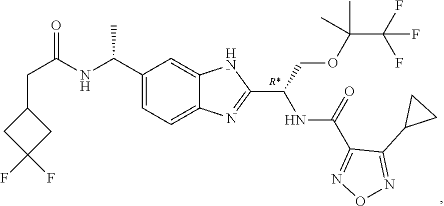 [SMILES: C[C@@H](NC(=O)CC1CC(F)(F)C1)c1ccc2nc([C@H](COC(C)(C)C(F)(F)F)NC(=O)c3nonc3C3CC3)[nH]c2c1]

or a pharmaceutically acceptable salt thereof.

In some embodiments, disclosed herein is a compound of Formula (I) having the following structure:

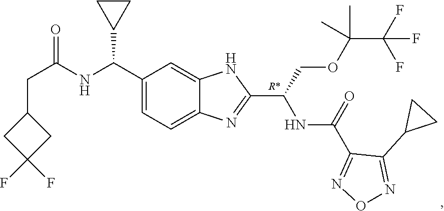 [SMILES: CC(C)(OC[C@H](NC(=O)c1nonc1C1CC1)c1nc2ccc([C@H](NC(=O)CC3CC(F)(F)C3)C3CC3)cc2[nH]1)C(F)(F)F]

or a pharmaceutically acceptable salt thereof.

In some embodiments, disclosed herein is a compound of Formula (I) having the following structure:

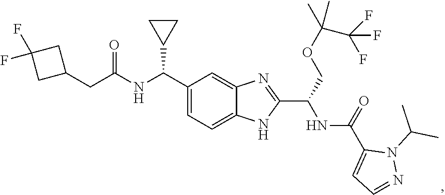 [SMILES: CC(C)n1nccc1C(=O)N[C@@H](COC(C)(C)C(F)(F)F)c1nc2cc([C@H](NC(=O)CC3CC(F)(F)C3)C3CC3)ccc2[nH]1]

or a pharmaceutically acceptable salt thereof.

In some embodiments, disclosed herein is a compound of Formula (I) having the following structure:

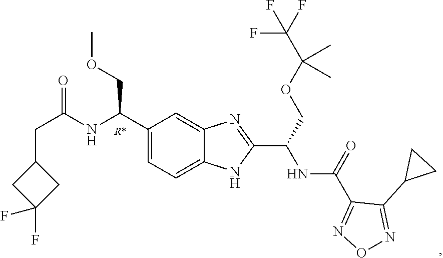 [SMILES: COC[C@H](NC(=O)CC1CC(F)(F)C1)c1ccc2[nH]c([C@H](COC(C)(C)C(F)(F)F)NC(=O)c3nonc3C3CC3)nc2c1]

or a pharmaceutically acceptable salt thereof.

In some embodiments, disclosed herein is a compound of Formula (I) having the following structure:

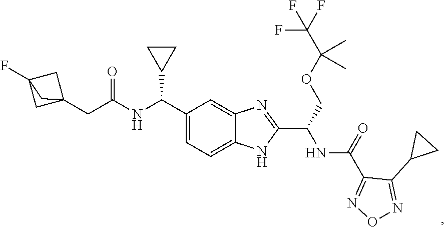 [SMILES: CC(C)(OC[C@H](NC(=O)c1nonc1C1CC1)c1nc2cc([C@H](NC(=O)CC34CC(F)(C3)C4)C3CC3)ccc2[nH]1)C(F)(F)F]

or a pharmaceutically acceptable salt thereof.

In some embodiments, disclosed herein is a compound of Formula (I) having the following structure:

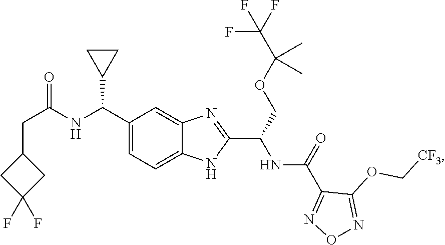 [SMILES: CC(C)(OC[C@H](NC(=O)c1nonc1OCC(F)(F)F)c1nc2cc([C@H](NC(=O)CC3CC(F)(F)C3)C3CC3)ccc2[nH]1)C(F)(F)F]

or a pharmaceutically acceptable salt thereof.

In some embodiments, disclosed herein is a compound of Formula (I) having the following structure:

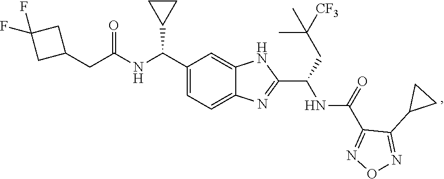 [SMILES: CC(C)(C[C@H](NC(=O)c1nonc1C1CC1)c1nc2ccc([C@H](NC(=O)CC3CC(F)(F)C3)C3CC3)cc2[nH]1)C(F)(F)F]

or a pharmaceutically acceptable salt thereof.

In some embodiments, disclosed herein is a compound of Formula (I) having the following structure:

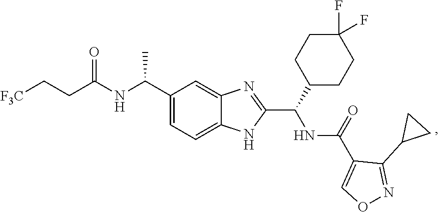 [SMILES: C[C@@H](NC(=O)CCC(F)(F)F)c1ccc2[nH]c([C@@H](NC(=O)c3conc3C3CC3)C3CCC(F)(F)CC3)nc2c1]

or a pharmaceutically acceptable salt thereof.

In some embodiments, disclosed herein is a compound of Formula (I) having the following structure:

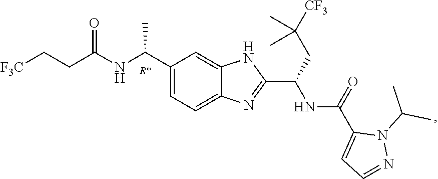 [SMILES: CC(C)n1nccc1C(=O)N[C@@H](CC(C)(C)C(F)(F)F)c1nc2ccc([C@@H](C)NC(=O)CCC(F)(F)F)cc2[nH]1]

or a pharmaceutically acceptable salt thereof.

In some embodiments, disclosed herein is a compound of Formula (I) having the following structure:

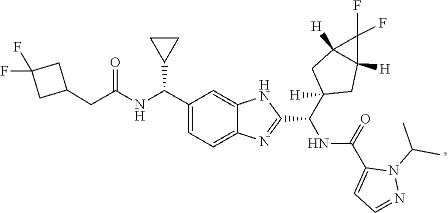 [SMILES: CC(C)n1nccc1C(=O)N[C@H](c1nc2ccc([C@H](NC(=O)CC3CC(F)(F)C3)C3CC3)cc2[nH]1)[C@@H]1C[C@@H]2[C@H](C1)C2(F)F]

or a pharmaceutically acceptable salt thereof.

In some embodiments, disclosed herein is a compound of Formula (I) having the following structure:

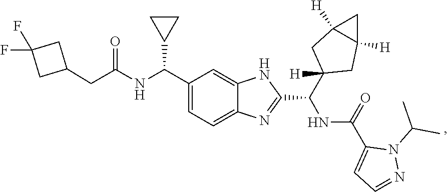 [SMILES: CC(C)n1nccc1C(=O)N[C@H](c1nc2ccc([C@H](NC(=O)CC3CC(F)(F)C3)C3CC3)cc2[nH]1)[C@@H]1C[C@H]2C[C@H]2C1]

or a pharmaceutically acceptable salt thereof.

In some embodiments, disclosed herein is a compound of Formula (I) having the following structure:

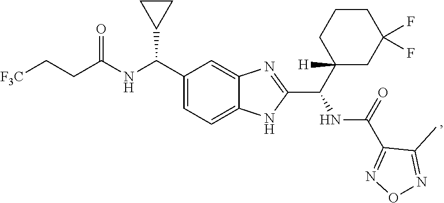 [SMILES: Cc1nonc1C(=O)N[C@H](c1nc2cc([C@H](NC(=O)CCC(F)(F)F)C3CC3)ccc2[nH]1)[C@@H]1CCCC(F)(F)C1]

or a pharmaceutically acceptable salt thereof.

In some embodiments, disclosed herein is a compound of Formula (I) having the following structure:

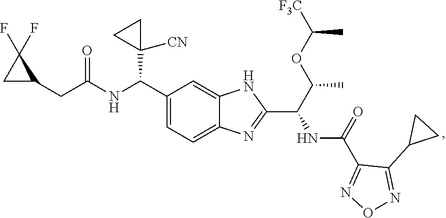

or a pharmaceutically acceptable salt thereof.

In some embodiments, disclosed herein is a compound of Formula (I) having the following structure:

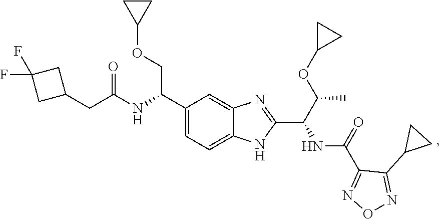 [SMILES: C[C@@H](OC1CC1)[C@H](NC(=O)c1nonc1C1CC1)c1nc2cc([C@@H](COC3CC3)NC(=O)CC3CC(F)(F)C3)ccc2[nH]1]

or a pharmaceutically acceptable salt thereof.

In some embodiments, disclosed herein is a pharmaceutical composition, comprising a compound of Formula (I), or a pharmaceutically acceptable salt thereof, and a pharmaceutically acceptable carrier. In some embodiments, the pharmaceutical composition is formulated for oral administration (e.g., a tablet or capsule).

In some embodiments, disclosed herein is a pharmaceutical composition made by mixing a compound of Formula (I), or a pharmaceutically acceptable salt thereof, and a pharmaceutically acceptable carrier.

In some embodiments, disclosed herein is a process for making a pharmaceutical composition comprising mixing a compound of Formula (I), or a pharmaceutically acceptable salt thereof, and a pharmaceutically acceptable carrier.

### III. Therapeutic Use

The present disclosure is also directed to a method for treating and/or ameliorating an IL-17 mediated inflammatory syndrome, disorder or disease comprising administering to a subject in need thereof an effective amount of a compound of Formula (I), or pharmaceutically acceptable salt thereof, composition thereof, or medicament thereof.

In some embodiments, disclosed herein is a method for treating or ameliorating an IL-17A mediated inflammatory syndrome, disorder, or disease comprising administering to a subject in need thereof a therapeutically effective amount of a compound of Formula (I), or a pharmaceutically acceptable salt thereof.

In some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigold, atopic dermatitis, vitiligo, multiple sclerosis, asthma, uveitis, chronic obstructive pulmonary disorder, multiple myeloma, and systemic lupus erythematosus.

IL-17A mRNA and/or protein levels are elevated in the lesional skin and blood of patients with psoriasis and correlate with disease severity. IL-17A acts directly in synergy with other cytokines (such as TNFα, IFNγ or IL-22) on keratinocytes triggering a self-amplifying inflammatory response in the skin and leading to the formation of psoriatic plaques. The blockade of IL-17A by means of antibodies to IL-17A or IL-23 results in complete reversal of the molecular and clinical disease features in majority of psoriasis patients, manifesting the significant role of IL-17A and IL-17-producing T-cells in the immunopathogenesis of psoriasis. (Hawkes et al., Psoriasis Pathogenesis and the Development of Novel, Targeted Immune Therapies. J Allergy Clin Immunol. 2017, 140(3): 645-653). The development and approval of IL-17 monoclonal antibodies such as secukinumab, ixekizumab, and brodalumab and their transformational efficacy for psoriasis have demonstrated IL-17A as a valid target for psoriasis treatments. (Blauvelt A. and Chiricozzi A. The Immunologic Role of IL-17 in Psoriasis and Psoriatic Arthritis Pathogenesis. Clin Rev Allergy Immunol. 2018, 55(3):379-390).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is psoriasis.

IL-17A is mechanistically relevant to psoriatic arthritis (PsA) through NFκB activation that triggers transcription of several PsA related genes including the receptor activator of nuclear factor KB ligand (RANKL). RANKL triggers the differentiation of osteoclast precursor cells into activated osteoclasts, resulting in bone resorption and subsequently joint deformity in PsA (Adamopoulos I. and Mellins E. Nature reviews Rheumatology 2015; 11:189-94). PsA joint is enriched for IL-17+CD8+ T cells, and the levels of this T cell subset are correlated with disease activity (Menon B. et al., Arthritis & Rheumatology 2014; 66: 1272-81). Synovial fibroblasts isolated from PsA patients also contain elevated IL-17R expression and secrete increased IL-6, CXCL8 and MMP3 ex vivo compared to osteoarthritis patients. Both secukinumab and ixekizumab are FDA approval drugs for PsA. In matching-adjusted indirect comparison analysis, secukinumab was associated with higher ACR 20/50/70 response rates in patients with active PsA than anti-TNFα antibodies (Mease P. et al., Eur. J. Rheumatol. 2018 Jul. 1; 6(3):113-121; Strand V. et al., J. Comp. Eff. Res. 2019, 8(7):497-510; Nash P. et al., Rheumatol. Ther. 2018, 5(1):99-122). In the most recent head-to-head study, ixekizumab was superior to adalimumab for improving signs and symptoms of active PsA (EULAR 2019 CONGRESS). By hitting the same target, IL-17A small molecule inhibitor compounds may exert similar or better efficacy than biologics considering that small molecules generally have better tissue penetration.

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is psoriatic arthritis.

IL-17A has been recognized as critical to the progression of rheumatoid arthritis (RA): “The recognition of IL-17 as a pro-inflammatory T cell derived cytokine, and its abundance within rheumatoid joints, provides the strongest candidate mechanism to date through which T cells can capture and localize macrophage effector functions in rheumatoid arthritis” Stamp, L. et al., Immunol. Cell Biol. 2004, 82(1): 1-9. Moreover, in rheumatoid arthritis IL-17A acts locally on synoviocytes and osteoblasts contributing to synovitis and joint destruction. Robert and Miossec have proposed the use of synovial biopsies and/or biomarkers to precisely identify patients that would respond to IL-17A inhibition. Their work concludes that IL-17 inhibitors should now be considered in the development of precision medicine in RA (Robert et al., Front. Med., 14 Jan. 2019).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is rheumatoid arthritis.

Various studies have reported elevated IL-17A and Th17 and other cells producing IL-17 in ankylosing spondylitis (AS) blood samples (Wendling D. et al., Joint Bone Spine. 2007; 74:304-305; Shen H. et al., Arthritis Rheum. 2009; 60(6):1647-56; Zhang L. et al., PLoS One. 2012; 7 (4):e31000; Jansen D. et al., Rheumatology (Oxford). 2015 April; 54(4): 728-735). In situ analysis of AS spine has revealed increased IL-17A-producing cells in bone of facet (zygapophyseal) joints (Appel H. et al., Arthritis Res. Ther. 2011; 13 (3):R95). Two advanced IL-17A neutralizing antibodies, secukinumab, approved by FDA for AS, and ixekizumab, have demonstrated efficacy over placebo even in anti-TNF inadequate responders. In contrast, anti-IL-23 p40 and p19 biologics failed to demonstrate beneficial effect (Deodhar A. et al., Arthritis Rheumatol. 2019, 71(2):258-270; BaetenD. et al., Ann. Rheum. Dis. 2018, 77(9):1295-1302), indicating the differential underling mechanism along IL-23/IL-17 pathway in AS and providing a strong evidence to support continuing developing IL-17A inhibitors.

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is ankylosing spondylitis.

Increased IL-17 and IL-17-producing T helper cells in the skin lesions of hidradenitis suppurativa (HS) patients were reported and molecular proteomics and gene expression data indicate that the IL-23/Th17 pathway is upregulated in HS lesions (Schlapbach C. et al., J. Am. Acad. Dermatol. 2011; 65(4):790; Kelly G. et al., British J. Dermatol. 2015 December; 173(6):1431-9; Moran B. et al., J. Invest. Dermatol. 2017; 137(11):2389; Thomi R. et al., JAMA Dermatol. 2018; 154(5):592). Seven of nine (78%) patients with moderate-to-severe HS achieved HiSCR in an open-label pilot-trial with Secukinumab (Prussick L. et al., British J. Dermatol. 2019 September; 181(3):609-611), and more clinical trials with anti-IL-17 mAbs in HS are on-going.

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is hidradenitis suppurativa.

IL-17 is elevated in the blister fluid and perilesional skin of bullous pemphigold (BP) patients. (Le Jan S. et al., J. Invest. Dermatol. 2014; 134 (12):2908-2917.; Chakievska L. J Autoimmun. 2019, 96:104-112). Exome sequencing of BP patients revealed mutations in twelve IL-17-related genes in one third of patients, providing the genetic link between IL-17 pathway and BP (Chakievska L. J Autoimmun. 2019, 96:104-112). In experimental murine BP, IL-17A−/−mice are protected, and anti-IL-17A treatment significantly reduced skin lesions in wild type (Chakievska L. J Autoimmun. 2019, 96:104-112). Ixekizumab Phase 2 of treatment naive and refractory BP patients is on-going (NCT03099538).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is bullous pemphigold.

IL-17 was found to be elevated in peripheral blood and lesions in atopic dermatitis (AD) patients and Th17 cells infiltrated more markedly in acute than chronic lesions, suggesting its role in acute phase of AD (Koga C. et al., J. Invest. Dermatol. 2008, 128, 2625-2630). Molecular profile analysis from ustekinumab Phase II suggest likely contribution of IL-23/Th17/IL-17 pathway in AD (Khattri S. et al., Exp. Dermatol. 2017 January; 26(1):28-35).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is atopic dermatitis.

Many studies in vitiligo patients have demonstrated an increased frequency of Th17 cells and higher level of IL-17 in both circulation and lesions that positively correlates with disease duration, extent, and activity (Singh R. et al., Autoimmun. Rev 2016, April; 15(4):397-404). Mouse studies demonstrated that depigmentation correlates with greater IL-17 expression/secretion, which modulates vitiligo development (Eby J. et al., Pigment Cell & Melanoma Res. 2014, November; 27(6):1075-85).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is vitiligo.

IL-17 expression is increased in PBMCs, cerebrospinal fluid (CSF) as well as in brain lesions and cells from multiple sclerosis (MS) patients (Lock, C. et al., Nat. Med. 2002, 8: 500-5085; Matusevicius, D. et al., Mult. Scler. 1999, 5: 101-104; Tzartos, J. et al., Am. J. Pathol. 2008, 172: 146-155). IL-17-producing T cells are enriched in active MS lesions (Tzartos, J. et al., Am. J. Pathol. 2008, 172: 146-155; Willing A. et al., J. Immunol. 2018, 200(3):974-982). IL-17A levels were elevated in the CSF of relapsing-remitting MS (RRMS) patients and correlated with the CSF/serum albumin quotient, a measure of blood-brain barrier (BBB) dysfunction, together with in vitro data that IL-17A in combination with IL-6 reduced the expression of tight junction-associated genes and disrupted monolayer integrity in a BBB cell line, highlighting the potential importance of targeting IL-17A in preserving BBB integrity in RRMS (Setiadi A F et al., J Neuroimmunol. 2019, 15; 332:147-154). Secukinumab yielded promising first results in a proof-of-concept study in MS patients (Havrdovi, E. et al., J. Neurol. 2016, 263: 1287-1295).

Accordingly, in some embodiments, disclosed herein is a method for treating or ameliorating and/an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is multiple sclerosis.

IL-17 expression is increased in the lung, sputum, bronchoalveolar lavage fluid, and sera in patients with asthma, and the severity of airway hyperresponsiveness is positively correlated with IL-17 expression levels. (Chakir J. et al., J. Allergy Clin. Immunol. 2003,111(6):1293-8). IL-17 was reported to be increased in asthmatic airways and induce human bronchial fibroblasts to produce cytokines (Molet S. et al., J. Allergy Clin. Immunol. 2001, 108(3):430-8). Anti-IL-17 antibody modulates airway responsiveness, inflammation, tissue remodeling, and oxidative stress in chronic mouse asthma models (Camargo LdN. et al., Front Immunol. 2018; 8: 1835; Dos Santos T. et al., Front. Physiol. 2018, 5; 9:1183).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is asthma.

IL-17 promotes the release of inflammatory mediators from retinal pigment epithelium cell line, disrupting the retinal pigment epithelium barrier function (Chen Y. et al., PLoS One. 2011; 6:e18139). IL-17 levels were elevated in the serum or aqueous humor of uveitis patients (El-Asrar A. et al., Clin. Immunol. 2011; 139(2):177-84; Jawad S. et al., Ocul. Immunol. Inflamm. 2013; 21(6):434-9; Kuiper J. et al., Am. J. Ophthalmol. 2011; 152(2):177-182.). Anti-IL-17 antibody delayed the onset of ocular inflammation and markedly inhibited the development of experimental autoimmune uveitis in rats (Zhang R. et al., Curr. Eye Res. 2009 April; 34(4):297-303). The analysis of secondary efficacy data from subcutaneous (sc) secukinumab phase 3 trials in uveitis suggested a beneficial effect of secukinumab in reducing the use of concomitant immunosuppressive medication (Dick A. et al., Ophthalmology 2013; 120(4):777-87). Later study of intravenous secukinumab in uveitis demonstrated greater efficacy than sc dosing, suggesting requiring optimal exposure for efficacy and confirming the therapeutic potential of IL-17A inhibition (Letko E. et al., Ophthalmology 2015, 122(5), 939-948). Ustekinumab that blocks IL-23/IL-17 pathway was also reported to successfully treat a noninfectious uveitis patient who had severe concomitant psoriasis and PsA and failed to respond to conventional immune suppressants (Mugheddu C. et al., Dermatol. Ther. 2017 September; 30(5)).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is uveitis.

An increase in Th17 cells was observed in patients with chronic obstructive pulmonary disorder (COPD) compared with current smokers without COPD and healthy subjects, and inverse correlations were found between Th17 cells with lung function (Vargas-Rojas M. et al., Respir. Med. 2011 November; 105(11):1648-54). In three recent human COPD studies, gene expression profile in bronchial epithelia showed that higher IL-17 signature expression is associated with a lack of response to inhaled corticosteroid, suggesting that there is a COPD subgroup that may benefit from IL-17 inhibitor therapy (Christenson S. et al., J. Clin. Invest. 2019; 129(1):169-181).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is chronic obstructive pulmonary disorder.

IL-17A serum levels were significantly higher in multiple myeloma (MM) patients and also in patients with advanced stage compared with healthy subjects (Lemancewicz D. et al., Med. Sci. Monit. 2012; 18 (1): BR54-BR59). Administration of secukinumab in the SCIDhu model of human myeloma weekly for 4 weeks after the first detection of tumor in mice led to a significant inhibition of tumor growth and reduced bone damage compared to isotype control mice (Prabhala R. et al., Leukemia. 2016 February; 30(2): 379-389).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is multiple myeloma.

Increased serum or plasma levels of IL-17, expansion of IL-17-producing T cells in the peripheral blood, and infiltration of Th17 cells in target organs like the kidneys in systematic lupus erythematosus (SLE) patients (Wong C. et al., Lupus. 2000; 9(8):589-593; Wong C. et al., Clinical Immunology. 2008; 127(3):385-393; Zhao X-F. et al., Mol. Biol. Rep. 2010 January; 37(1):81-5; Chen X. et al., J. Clin. Immunol. 2010 March; 30(2):221-5; Xing Q. et al., Rheumatol. Int. 2012 April; 32(4):949-58). Imbalance between Th17 cells and regulatory T (Treg) cells has been observed in SLE patients including quiescent stage (Ma J. et al., Clin. Rheumatol. 2010; 29(11):1251-1258; Dolff S. et al., Clin. Immunol. 2011, 141(2):197-204). Overexpression of IL-17A using adenovirus enhanced the severity of lupus nephritis, while blockade of IL-17A using neutralizing antibody resulted in decreased severity of lupus nephritis (Wen, Z. et al., PLoS One. 2013, 8: e58161). In a phase 2 study, ustekinumab, an anti-IL-12/23 p40 monoclonal antibody blocking IL-23/IL-17 pathway, has demonstrated efficacy in SLE patients (Vollenhoven R. et al., Lancet 2018; 392: 1330-39). Human expression studies, animal models, and clinical trials indicate that IL-17 blockade may become a promising therapeutic strategy for SLE (Koga T. et al., Expert Rev. Clin. Immunol. 2019, 15 (6) 629-637).

Accordingly, in some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is systemic lupus erythematosus.

In some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigold, atopic dermatitis, vitiligo, multiple sclerosis, asthma, uveitis, chronic obstructive pulmonary disorder, multiple myeloma, and systemic lupus erythematosus, wherein the compound of Formula (I) or the pharmaceutically acceptable salt thereof is administered orally (e.g., as a tablet or capsule).

In some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigold, atopic dermatitis, vitiligo, multiple sclerosis, asthma, uveitis, chronic obstructive pulmonary disorder, multiple myeloma, and systemic lupus erythematosus, wherein the therapeutically effective amount is a dose of about 10 mg to 300 mg QD. In some embodiments, the therapeutically effective amount is a dose of about 20 mg to 200 mg QD. In some embodiments, the therapeutically effective amount is a dose of about 50 mg to 100 mg QD.

In some embodiments, disclosed herein is a method for treating and/or ameliorating an IL-17A mediated inflammatory syndrome, disorder, or disease comprising administering to a subject in need thereof a therapeutically effective amount of a compound of Formula (I), or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigold, atopic dermatitis, vitiligo, multiple sclerosis, asthma, uveitis, chronic obstructive pulmonary disorder, multiple myeloma, and systemic lupus erythematosus, wherein the therapeutically effective amount is a dose of about 10 mg to 300 mg BID. In some embodiments, the therapeutically effective amount is a dose of about 20 mg to 200 mg BID. In some embodiments, the therapeutically effective amount is a dose of about 50 mg to 100 mg BID.

In some embodiments, disclosed herein is the use of a therapeutically effective amount of compound of Formula (I), or a pharmaceutically acceptable salt thereof, for treating and/or ameliorating an IL-17A mediated inflammatory syndrome, disorder, or disease selected from the group consisting of: psoriasis, psoriatic arthritis, rheumatoid arthritis, ankylosing spondylitis, hidradenitis suppurativa, bullous pemphigold, atopic dermatitis, vitiligo, multiple sclerosis, asthma, uveitis, chronic obstructive pulmonary disorder, multiple myeloma, and systemic lupus erythematosus.

In some embodiments, disclosed herein is the use of a compound of Formula (I), or pharmaceutically acceptable salt thereof, in the manufacture of a medicament for treating and/or ameliorating an IL-17A mediated inflammatory syndrome, disorder, or disease selected from the group consisting of: psoriasis, psoriatic arthritis, rheumatoid arthritis, ankylosing spondylitis, hidradenitis suppurativa, bullous pemphigold, atopic dermatitis, vitiligo, multiple sclerosis, asthma, uveitis, chronic obstructive pulmonary disorder, multiple myeloma, and systemic lupus erythematosus.

In some embodiments, disclosed herein is a method for treating and/or ameliorating an IL-17 mediated inflammatory syndrome, disorder or disease comprising administering to a subject in need thereof a therapeutically effective amount of a compound of Formula (I), or pharmaceutically acceptable salt thereof, a composition thereof, or a medicament thereof.

In some embodiments, disclosed herein is a method of treating and/or ameliorating an IL-17 mediated inflammatory syndrome, disorder or disease, wherein the syndrome, disorder or disease is selected from the group consisting of: psoriasis, psoriatic arthritis, rheumatoid arthritis, ankylosing spondylitis, hidradenitis suppurativa, atopic dermatitis, vitiligo, multiple sclerosis, asthma, allergic asthma, steroid resistant asthma, neutrophilic asthma, chronic obstructive pulmonary disease, uveitis, multiple myeloma, and systemic lupus erythematosus, comprising administering to a subject in need thereof a therapeutically effective amount of a compound of Formula (I), or pharmaceutically acceptable salt thereof, a composition thereof, or a medicament thereof.

In some embodiments, disclosed herein is a method of treating or ameliorating an IL-17 mediated inflammatory syndrome, disorder or disease, wherein the syndrome, disorder or disease is selected from the group consisting of: psoriasis, psoriatic arthritis, and ankylosing spondylitis, comprising administering to a subject in need thereof a therapeutically effective amount of a compound of Formula (I), or pharmaceutically acceptable salt thereof, a composition thereof, or a medicament thereof.

In some embodiments, disclosed herein are methods of modulating IL-17 activity in a mammal by administration of a therapeutically effective amount of at least one compound of Formula (I), or pharmaceutically acceptable salt thereof.

Also disclosed herein is a method of inhibiting production of interleukin-17, comprising administering to a subject in need thereof a therapeutically effective amount of a compound of Formula (I), or pharmaceutically acceptable salt thereof.

### Combination Therapy

A compound of Formula (I), or pharmaceutically acceptable salt thereof, a composition thereof, or a medicament thereof may also be used in combination with one or more additional therapeutic agents.

In some embodiments, the one or more additional therapeutic agents is selected from the group consisting of anti-inflammatory agents, immunomodulatory agents, and immunosuppressive agents.

In some embodiments, the one or more additional therapeutic agents is selected from the group consisting of:
(a) anti-TNFalpha agents such as infliximab (Remicade®), adalimumab (Humira®), certolizumab pegol (Cimzia®), golimumab (Simponi®), etanercept (Enbrel®), thalidomide (Immunoprin®), lenalidomide (Revlimid®), and pomalidomide (Pomalyst®/Imnovid®);
(b) anti-p40 antibody agents such as ustekinumab (Stelara®); and
(c) anti-p19 antibody agents such as guselkumab (Tremfya®), tildrakizumab (Ilumya™/Ilumetri), risankizumab (Skyrizi™), and mirikizumab.

In some embodiments, disclosed herein is a method of treating and/or ameliorating an IL-17 mediated inflammatory syndrome, disorder or disease, in a subject in need thereof comprising administering to the subject a therapeutically effective amount of the compound of Formula (I), or pharmaceutically acceptable salt thereof, composition thereof, or medicament thereof in a combination therapy with one or more additional therapeutic agents, such as anti-inflammatory agents, immunomodulatory agents, or immunosuppressive agents, wherein said syndrome, disorder or disease is selected from the group consisting of psoriasis, psoriatic arthritis, rheumatoid arthritis, ankylosing spondylitis, hidradenitis suppurativa, atopic dermatitis, vitiligo, multiple sclerosis, asthma, allergic asthma, steroid resistant asthma, neutrophilic asthma, chronic obstructive pulmonary disease, uveitis, multiple myeloma, and systemic lupus erythematosus.

In some embodiments, disclosed herein is a method of treating and/or ameliorating an IL-17 mediated inflammatory syndrome, disorder or disease, in a subject in need thereof comprising administering to the subject a therapeutically effective amount of the compound of Formula (I), or pharmaceutically acceptable salt thereof, composition thereof, or medicament thereof in a combination therapy with one or more additional therapeutic agents, such as anti-inflammatory agents, or immunosuppressive agents, wherein said syndrome, disorder or disease is psoriasis, psoriatic arthritis, ankylosing spondylitis. In some embodiments, the IL-17 mediated inflammatory syndrome, disorder or disease is psoriasis. In some embodiments, the IL-17 mediated inflammatory syndrome, disorder or disease is psoriatic arthritis. In some embodiments, the IL-17 mediated inflammatory syndrome, disorder or disease is ankylosing spondylitis.

### Dosage Regimen

When employed as IL-17A modulators, the compounds disclosed herein may be administered in an effective amount within the dosage range of about 0.5 mg to about 1 g, preferably between about 0.5 mg to about 500 mg, in single or divided daily doses. In some embodiments, the dosage amount is about 5 mg to 400 mg. In some embodiments, the dosage amount is about 10 mg to 300 mg. In some embodiments, the dosage amount is about 0.5, 1, 5, 10, 15, 20, 25, 30, 35, 40, 45, 50, 55, 60, 65, 70, 75, 80, 85, 90, 95, or 100 mg of a compound of Formula (I), or pharmaceutically acceptable salt thereof. In some embodiments, the dosage amount is about 100, 105, 110, 115, 120, 125, 130, 135, 140, 145, 150, 155, 160, 165, 170, 175, 180, 185, 190, 195, or 200 mg of a compound of Formula (I), or pharmaceutically acceptable salt thereof. In some embodiments, the dosage amount is about 200, 205, 210, 215, 220, 225, 230, 235, 240, 245, 250, 255, 260, 265, 270, 275, 280, 285, 290, 295, or 300 mg of a compound of Formula (I), or pharmaceutically acceptable salt thereof. In some embodiments, the dosage amount is about 300, 315, 320, 325, 330, 335, 340, 345, 350, 355, 360, 365, 370, 375, 380, 385, 390, 395, or 400 mg of a compound of Formula (I), or pharmaceutically acceptable salt thereof. In some embodiments, the dosage amount is about 400, 405, 410, 415, 420, 425, 430, 435, 440, 445, 450, 455, 460, 465, 470, 475, 480, 485, 490, 495, or 500 mg of a compound of Formula (I), or pharmaceutically acceptable salt thereof.

In some embodiments, a compound of Formula (I), or pharmaceutically acceptable salt thereof, may be administered in an effective amount within the dosage range of about 10 mg to 300 mg QD. In some embodiments, a compound of Formula (I), or pharmaceutically acceptable salt thereof, may be administered in an effective amount within the dosage range of about 20 mg to 200 mg QD. In some embodiments, a compound of Formula (I), or pharmaceutically acceptable salt thereof, may be administered in an effective amount within the dosage range of about 50 mg to 100 mg QD.

In some embodiments, a compound of Formula (I), or pharmaceutically acceptable salt thereof, may be administered in an effective amount within the dosage range of about 10 mg to 300 mg BID. In some embodiments, a compound of Formula (I), or pharmaceutically acceptable salt thereof, may be administered in an effective amount within the dosage range of about 20 mg to 200 mg BID. In some embodiments, a compound of Formula (I), or pharmaceutically acceptable salt thereof, may be administered in an effective amount within the dosage range of about 50 mg to 100 mg BID.

The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present invention or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

### Pharmaceutically Acceptable Salts

The present disclosure also includes pharmaceutically acceptable salt forms of the compounds described herein. Lists of suitable pharmaceutically acceptable salts are found in Remington's Pharmaceutical Sciences, 17th ed., Mack Publishing Company, Easton, Pa., 1985, p. 1418, the disclosure of which is hereby incorporated by reference in its entirety. Pharmaceutically acceptable salts include the conventional non-toxic salts or the quaternary ammonium salts which are formed from inorganic or organic acids or bases. Examples of such salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Further acceptable salts include ammonium salts, alkali metal salts such as sodium and potassium salts, alkaline earth metal salts such as calcium and magnesium salts, salts with organic bases such as dicyclohexylamino salts and salts with amino acids such as arginine.

### Pharmaceutical Compositions

The compounds of Formula (I), or pharmaceutically acceptable salts thereof, may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutical compositions of the invention may be administered by any means that accomplish their intended purpose. Examples include administration by parenteral, subcutaneous, intravenous, intramuscular, intraperitoneal, transdermal, topical, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.

Also disclosed herein is a method of making a pharmaceutical composition comprising mixing a pharmaceutically acceptable carrier with any of the compounds of Formula (I), or pharmaceutically acceptable salt thereof. Additionally, the present application includes pharmaceutical compositions made by mixing a pharmaceutically acceptable carrier with any of the compounds of the present invention.

### EXAMPLES

### Abbreviations

Herein and throughout the application, the following abbreviations may be used.
Ac acetyl
ACN acetonitrile
Boc tert-butyloxycarbonyl
BOP benzotriazol-1-yloxytris(dimethylamino)phosphonium
hexafluorophosphate
br broad
Bs p-bromobenzenesulfonyl
Bu butyl
CDI 1,1′-carbonyldiimidazole
δ NMR chemical shift in parts per million downfield from a TMS standard
d doublet (coupling pattern) or day(s)
DABCO 1,4-diazabicyclo[2.2.2]octane
DAST diethylaminosulfur trifluoride
dba dibenzylidineacetone
DBAD di-tert-butyl azodicarboxylate
DCC dicyclohexylcarbodiimide
DCM dichloromethane
dd doublet of doublets
Deoxo-Fluor® bis(2-methoxyethyl)aminosulfur trifluoride
Dess-Martin Periodinane 1,1,1-tris(acetyloxy)-1,1-dihydro-1,2-benziodoxol-3-(1H)-one
DIAD diisopropyl azodicarboxylate
DIBAL-H diisobutylaluminum hydride
DIPEA N,N-diisopropylethylamine (Hünig's base)
DMA N,N-dimethylacetamide
DMAP 4-dimethylaminopyridine
DMF N,N-dimethylformamide
DMSO dimethyl sulfoxide
dppf 1,1′-bis(diphenylphosphino)ferrocene
EDCI 1-ethyl-3-(3-dimethylaminopropyl) carbodiimide hydrochloride
ESI electrospray ionization
Et ethyl
EtOAc ethyl acetate
g gram(s)
h hour(s)
HATU 1-[bis(dimethylamino)methylene]-1H-1,2,3-triazolo[4,5-b]pyridinium 3-oxide hexafluorophosphate
HBTU 3-[bis(dimethylamino)methyliumyl]-3H-benzotriazol-1-oxide hexafluorophosphate
HOAt 1-hydroxy-7-azabenzotriazole
HOBt 1-hydroxybenzotriazole
HPLC high pressure liquid chromatography
HMPA hexamethylphosphoramide
Hz Hertz
i iso
IPA isopropanol
J coupling constant in Hertz (NMR spectroscopy)
Jones reagent CrO₃, H₂SO₄, acetone
L liter(s)
LAH lithium aluminum hydride
Lawesson's reagent 2,4-bis(4-methoxyphenyl)-1,3,2,4-dithiadiphosphetane-2,4-dithione
LC liquid chromatography
LDA lithium diisopropylamide
LED light-emitting diode
m milli or multiplet
m/z mass-to-charge ratio
M⁺ parent molecular ion
M molar (moles/liter)
Me methyl
mCPBA 3-chloroperbenzoic acid
MeCN acetonitrile
min minute(s)
MFSDA methyl fluorosulfonyldifluoroacetate
μ micro
MS mass spectrometry or molecular sieves
MTBE tert-butyl methyl ether
n normal
N normal (equivalent concentration)
NCS N-chlorosuccinimide
NMR nuclear magnetic resonance
NMP N-methyl-2-pyrrolidone
Pd/C palladium on carbon
Ph phenyl
PPTS pyridinium p-toluenesulfonate
Pr propyl
psi pounds per square inch
PyBOP benzotriazol-1-yloxy)tripyrrolidinophosphonium
hexafluorophosphate
PyBroP bromotri(pyrrolidin-1-yl)phosphonium hexafluorophosphate
q quartet
quin quintet
Rochelle salt sodium potassium tartrate
rt room temperature
s singlet
SEM 2-(trimethylsilyl)ethoxymethyl
SFC supercritical fluid chromatography
t tert
t triplet
TBAF tetrabutylammonium fluoride
TBD 1,5,7-triazabicyclo[4.4.0]dec-5-ene
TFA trifluoroacetic acid
THF tetrahydrofuran
Ts 4-toluenesulfonyl
T3P® propanephosphonic acid anhydride
XPhos 2-dicyclohexylphosphino-2′,4′,6′-triisopropylbiphenyl

In some embodiments, provided herein are processes and intermediates disclosed herein that are useful for preparing a compound of Formula (I) or pharmaceutically acceptable salts thereof.

In the schemes below, an amide bond may be formed using the following methods: (1) the reaction of a suitably substituted amine can be reacted with a suitably substituted carboxylic acid. The carboxylic acid is activated with an appropriate activating reagent, for example a carbodiimide, such as DCC, CDI or EDCI optionally in the presence of HOBt or HOAt and/or a catalyst such as DMAP or N-methylimidazole; a halotrisaminophosphonium salt such as BOP, PyBOP, or PyBroP; a suitable pyridinium salt such as 2-chloro-1-methyl pyridinium chloride; or another suitable coupling agent such as HBTU, HATU, 2,4,6-tripropyl-1,3,5,2,4,6-trioxatriphosphorinane-2,4,6-trioxide (T3P®) and like. Coupling reactions are conducted in a suitable solvent such as DCM, THF, DMF, ACN or mixtures thereof, optionally in the presence of a tertiary amine such as N-methylmorpholine, pyridine, diisopropylethyl amine, or triethylamine, at a temperature ranging from about 0° C. to about the reflux temperature of the solvent or solvent mixture; (2) the reaction of a suitably substituted amine can be reacted with a suitably substituted carboxylic acid derivative, such as a carboxylic acid chloride (acid chloride), a carboxylic acid anhydride, a carboxylic acid ester, or a carboxylic acid N-hydroxysuccinate ester. The carboxylic acid derivative (such as a carboxylic acid chloride, a carboxylic acid anhydride, or a N-hydroxysuccinate ester) is reacted with a suitably substituted amine in a suitable solvent such as DCM, THF, DMF, ACN, or mixtures thereof, optionally in the presence of a tertiary amine such as N-methylmorpholine, diisopropylethyl amine, pyridine, or triethylamine, at a temperature ranging from about 0° C. to about the reflux temperature of the solvent or solvent mixture. In addition, a carboxylic acid ester is reacted with a suitably substituted amine the presence of reagents such as trimethylaluminum in solvents such as toluene, or in solvents such as 2,2,2-trifluoroethanol or DMA to form the amide bond. The aforementioned conditions are referred to as “amide bond forming conditions”.

In the schemes below, SEM protecting groups can be removed using reagents such as (1) hydrochloric acid or TFA in solvents such as DCM, methanol, 1,4-dioxane, EtOAc or mixtures thereof, (2) PPTS in IPA at a temperature ranging from about rt to about reflux temperature of the solvent or (3) TBAF in THE at a temperature ranging from about rt to about reflux temperature of the solvent. The aforementioned conditions are referred to as “SEM deprotection conditions”.

In the schemes below, the sulfinamide group can be removed using conditions such as the following: (1) hydrochloric acid in solvents such as EtOAc, 1,4-dioxane, THF, water or mixtures thereof or (2) iodine in solvents such as THF, water or mixtures thereof. The aforementioned conditions are referred to as “sulfinamide deprotection conditions”.

In the schemes below, the term “LG” is used as an abbreviation for leaving group. Examples of leaving groups include: —Br, —Cl, —I, methane sulfonate, p-bromobenzenesulfonate and 4-toluenesulfonate.

In the schemes below, the SEM group is shown as a protecting group for the benzimidazole nitrogen. When SEM isomers are depicted on the nitrogen on the N1 of the benzimidazole ring (e.g. see structure below, B-V), it may represent a mixture of structural isomers as shown by structures B-Va and B-Vb.

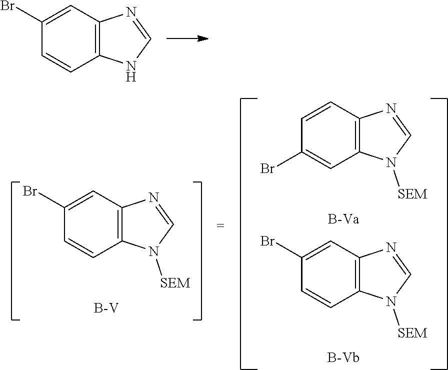

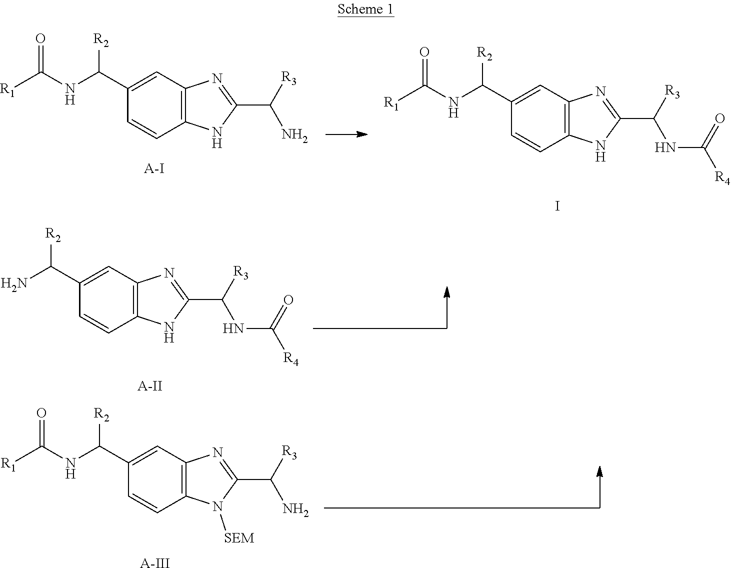

The compounds of Formula (I) in the present invention can be prepared according to Scheme 1. Amines A-I and carboxylic acids R⁴—CO₂H (A-Ia), carboxylic acid chlorides R⁴—COCl (A-Ib), carboxylic esters R⁴—CO₂Me (A-Ic) and carboxylic acid N-hydroxysuccinate esters R⁴—N-hydroxysuccinate esters (A-Id) can be reacted using applicable amide bond forming conditions to yield compounds of Formula (I). Alternatively, reaction between amines A-II and carboxylic acids R¹—CO₂H (A-IVa) or carboxylic acid chlorides R¹—COCl (A-IVb) using applicable amide bond forming conditions yields compounds of Formula (I). In addition, amines A-III can be coupled with compounds A-Ia, A-Ib, A-Ic or A-Id using applicable amide bond conditions to afford the corresponding amides (structure not shown). Subsequent deprotection of the SEM protecting group using SEM deprotection conditions affords compounds of Formula (I).

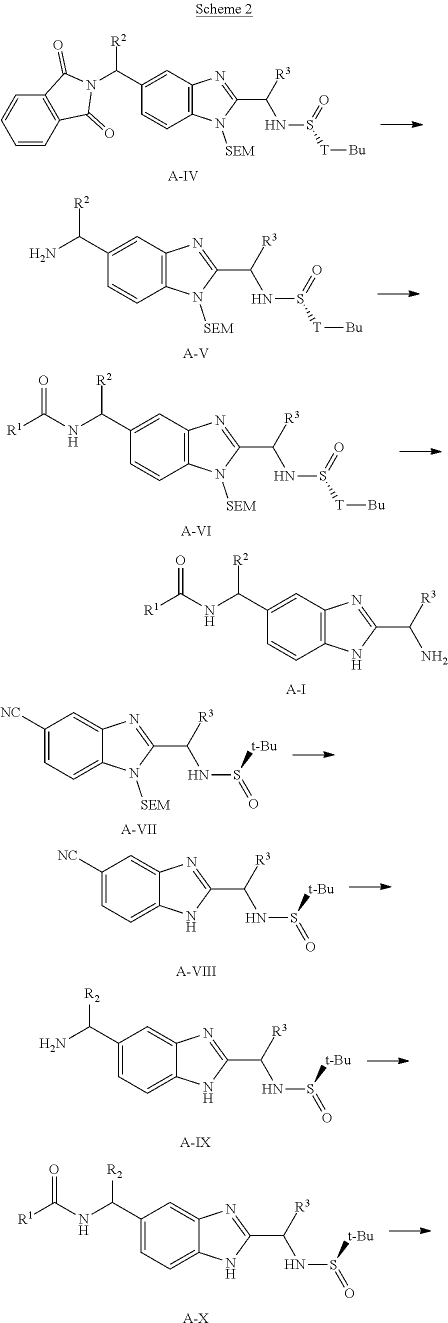

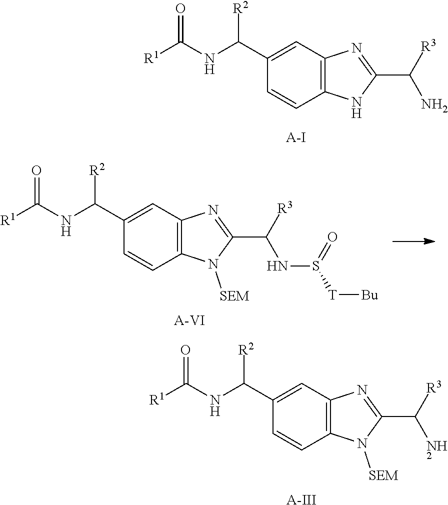

The synthesis of amines A-I and A-III is shown in Scheme 2. Deprotection of the phthalimide group within compounds A-IV using reagents such as hydrazine in solvents such as ethanol affords amines A-V. Amides A-VI can be prepared by reaction of amines A-V with A-IVa or A-IVb using amide bond forming conditions. Treatment of compounds A-VI with reagents such as hydrochloric acid in a solvent such as methanol or 1,4-dioxane and EtOAc yields amines A-I. Alternatively a two-step deprotection can be performed wherein compounds A-VI are first treated with reagents such as hydrochloric acid in 1,4-dioxane to remove the sulfinamide protecting group, and in a second step the SEM group is removed using reagents such as TFA to afford compounds A-I. Compounds A-I can also be prepared starting from nitriles A-VII. Deprotection of the SEM group using a reagent such as TBAF in a solvent such as THE affords nitriles A-VIII. Subsequent treatment of nitriles A-VIII with suitable Grignard reagents, such as R²—MgBr or R²—MgCl, in the presence of copper salts such as CuI or CuCl in solvents such as THE followed by reduction using reagents such as sodium borohydride in solvents such as methanol afford amines A-IX. Amide bond coupling between A-IX and compounds such as A-IVa or A-IVb using amide bond forming conditions affords amides A-X. Deprotection of the sulfinamide group within A-X using sulfinamide deprotection conditions affords intermediates A-I. Sulfinamides of the general formula A-VI can be treated with reagents such as hydrochloric acid in 1,4-dioxane or iodine in solvents such as THE and water to generate compounds A-III.

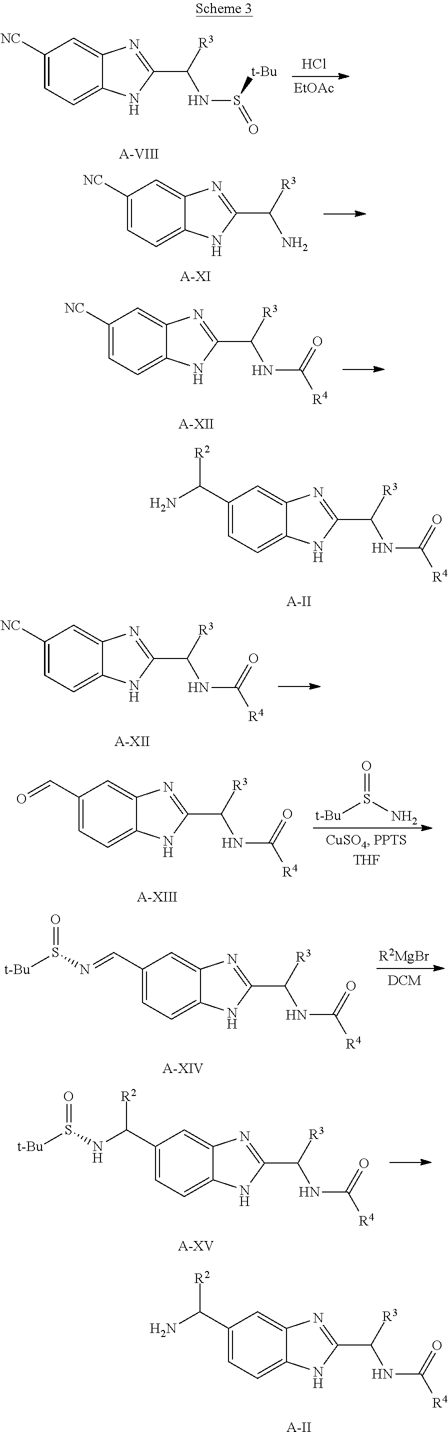

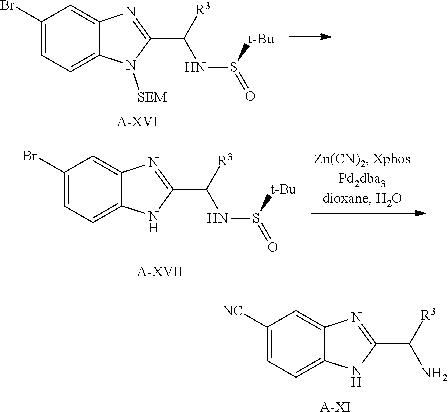

Scheme 3 shows the synthesis of amines A-II. Deprotection of the sulfinamide within A-VIII using sulfinamide deprotection conditions provides amines A-XI. Reaction of compound A-XI with compounds A-Ia or A-Ib using applicable amide bond forming conditions affords amides A-XII. Treatment of the nitrile within A-XII with suitable Grignard reagents, such as R²—MgBr or R²—MgCl, in the presence of additives such as CuI or CuCl in solvents such as THF followed by reduction using reagents such as sodium borohydride in solvents such as methanol affords benzylic amines A-II. Alternatively, amines A-II can be prepared by reduction of nitriles A-XII using a reagent such as Raney®-Nickel in solvents such as pyridine and acetic acid in the presence of additives such as sodium hypophosphite monohydrate to afford aldehydes of the general formula A-XIII. Condensation with (S)-2-methylpropane-2-sulfinamide in the presence of reagents such as copper sulfate and PPTS in a solvent such as THE provides sulfinimines of the general formula A-XIV. Subsequent addition of a suitable Grignard reagent, such as R²—MgBr or R²—MgCl, in a solvent such as DCM then affords sulfinamides of the general formula A-XV. Deprotection of the sulfinamide within compounds of formula A-XV using sulfinamide deprotection conditions affords amines of the general formula A-II. Nitrile intermediate A-XI can also be prepared by an alternative sequence that initiates with bromides A-XVI. Deprotection of the SEM group within A-XVI using SEM deprotection conditions yields bromides A-XVII. Subsequent cyanation using reagents such as dicyanozinc in the presence of catalysts such as Pd₂dba₃ and ligands such as XPhos in solvents such as 1,4-dioxane and water affords compounds A-XI.

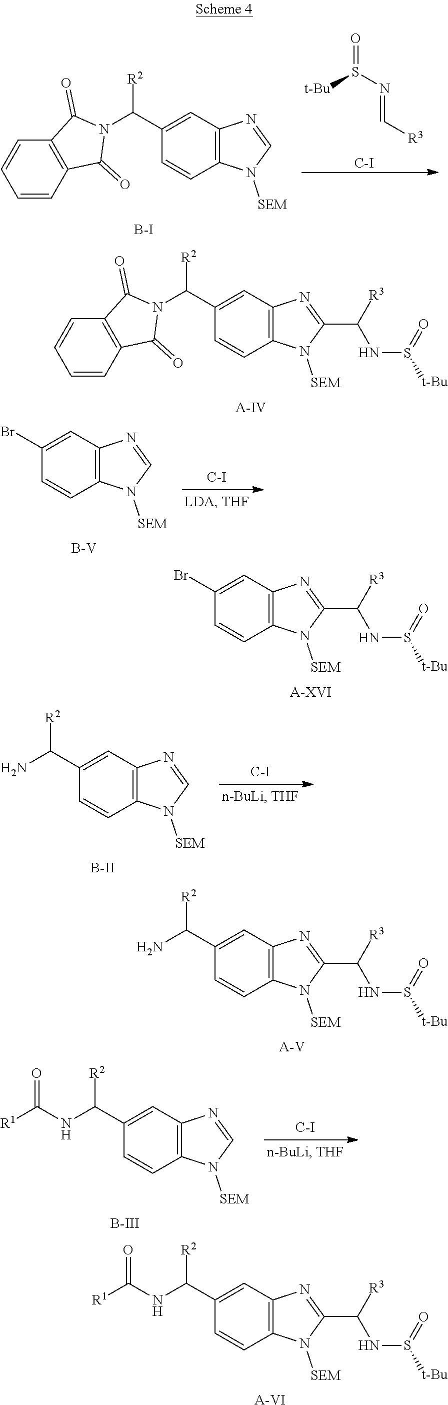

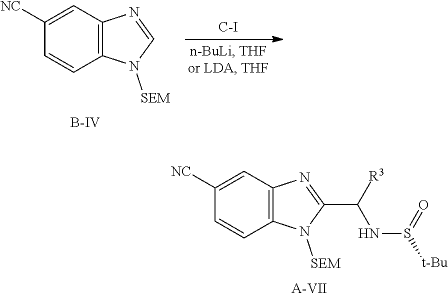

Sulfinamides A-IV, A-V, A-VI, A-VII and A-XVI may be prepared as shown in Scheme 4. Deprotonation of benzimidazoles B-I with a base such as LDA in a solvent such as THF, followed by reaction with sulfinimines C-I provides compounds A-IV. Deprotonation of the benzimidazole B-V with a base such as LDA in a solvent such as THF, followed by reaction with sulfinimines C-I provides compounds A-XVI. Deprotonation of benzimidazoles B-II with a base such as n-BuLi in a solvent such as THF, followed by reaction with sulfinimines C-I provides compounds A-V. Deprotonation of benzimidazoles B-III with a base such as n-BuLi in a solvent such as THF, followed by reaction with sulfinimines C-I provides compounds A-VI. Deprotonation of benzimidazole B-IV with a base such as n-BuLi or LDA in a solvent such as THF, followed by reaction with sulfinimines C-1 provides compounds A-VII.

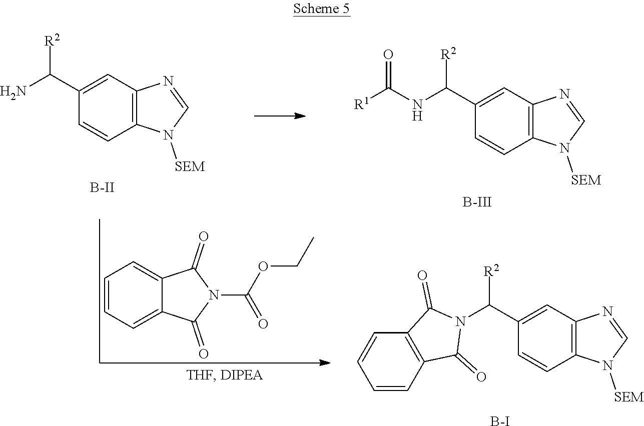

Benzimidazoles B-I and B-III are prepared as shown in Scheme 5. Reaction between amines B-II and compounds such as A-IVa or A-IVb using amide bond forming conditions affords the corresponding amides B-III. Protection of the amine within B-II using ethyl 1,3-dioxoisoindoline-2-carboxylate in solvents such as THF in the presence of additives such as DIPEA generates phthalimides B-I.

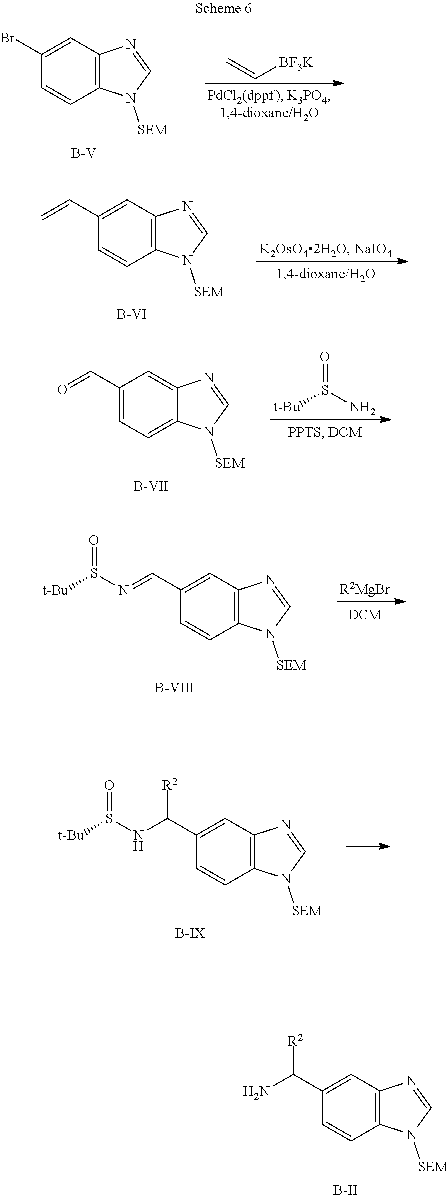

Benzimidazoles B-II can be prepared as shown in Scheme 6. Vinylation of bromide B-V using reagents such as potassium trifluoro(vinyl)boranide in the presence of palladium catalysts such as PdCl₂dppf and bases such as potassium phosphate in solvents such as 1,4-dioxane and water affords vinylated intermediate B-VI. Oxidative cleavage of the olefin within B-VI using reagents such as potassium osmate dihydrate and sodium periodate in solvents such as 1,4-dioxane and water affords the corresponding aldehyde B-VII. Condensation of aldehyde B-VII with (S)-2-methylpropane-2-sulfinamide in the presence of additives such as PPTS and copper sulfate in solvents such as DCM then provides the corresponding sulfinimide B-VIII. Sulfinimide B-VIII can be exposed to suitable Grignard reagents such as R²—MgBr or R²—MgCl in solvents such as DCM to provide sulfinamides B-IX. Subsequent deprotection of the sulfinamide group within B-IX using sulfinamide deprotection conditions affords amines B-II.

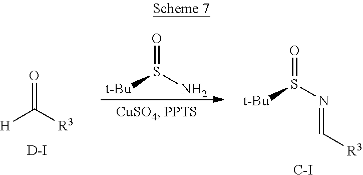

Sulfinimides C-I are prepared as shown in Scheme 7. Condensation of aldehydes D-I with sulfimamides such as (R)-2-methylpropane-2-sulfinamide in the presence of reagents such as copper sulfate and PPTS in solvents such as DCM, THE and/or toluene yields sulfinimines C-I.

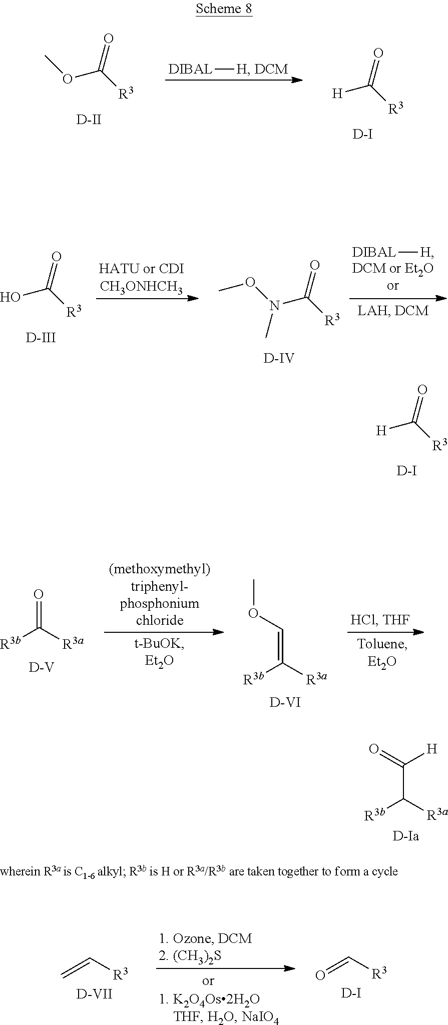

Aldehydes D-I are prepared as shown in Scheme 8. In some cases, carboxylic esters D-II can be reduced using reagents such as DIBAL-H in solvents such as DCM to afford aldehydes D-I. Carboxylic acids D-III can be converted to the corresponding amides (D-IV) by treatment with N,O-dimethylhydroxylamine in the presence of reagents such as HATU or CDI and additives such as DIPEA in solvents such as DCM. Reduction of amides D-IV using reducing agents such as DIBAL-H or lithium aluminum hydride in solvents such as DCM or ethyl ether afford the corresponding aldehydes D-I. Aldehydes or ketones D-V can be treated with (methoxymethyl)triphenylphosphonium chloride in the presence of a base such as potassium tert-butoxide in solvents such as ethyl ether to afford methyl enol ethers D-VI. Hydrolysis of enol ethers D-VI using reagents such as hydrochloric acid in solvents such as THF, toluene or ethyl ether reveal aldehydes of the structure D-Ia. Terminal olefins D-VII may be cleaved under oxidative conditions by treatment with reagents such as ozone in DCM followed by treatment with dimethyl sulfide or by treatment with reagents such as potassium osmate dihydrate and sodium periodate in solvents such as THE and water to afford aldehydes D-I.

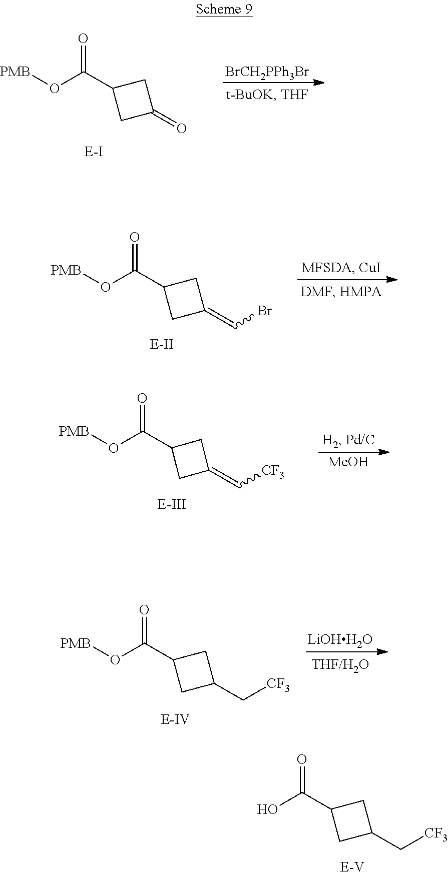

3-(2,2,2-Trifluoroethyl)cyclobutene-1-carboxylic acid E-V can be prepared as shown in Scheme 9. Treatment of ketone E-I with the reagent prepared from the reaction of (bromomethyl)triphenylphosphonium bromide and potassium t-butoxide in THE affords the corresponding vinyl bromide E-II. Trifluoromethylation can be achieved by treatment of E-II with MFSDA in the presence of copper iodide in DMF and HMPA to yield E-III. Reduction of the olefin within E-III is achieved by treatment with hydrogen gas in the presence of palladium on carbon in methanol to afford the compound of the structure E-IV. Saponification of the ester within E-IV using aqueous LiOH in THE provides carboxylic acid E-V.

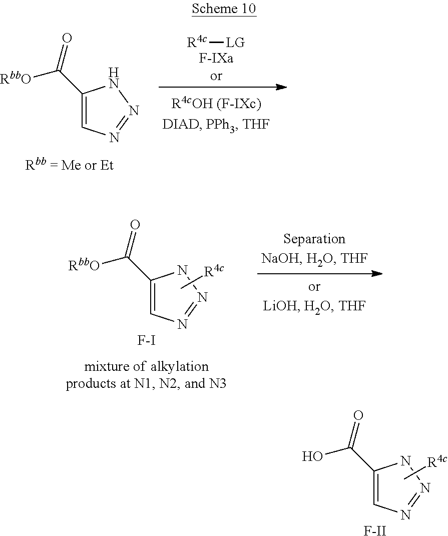

Substituted 1,2,3-triazole-5-carboxylic acids F-II can be prepared as shown in Scheme 10. Methyl or ethyl 1H-1,2,3-triazole-5-carboxylate can be alkylated by treating the ester with a base such as potassium carbonate or sodium hydride and a compound of general structure R^{4c}-LG (F-IXa) in a solvent such as DMF to yield a mixture of 1,2,3-triazole-5-carboxylates F-I alkylated at the N1, N2 or N3 positions that could be separated by silica gel chromatography. Alternatively, alkylation can be accomplished by treatment of methyl or ethyl 1H-1,2,3-triazole-5-carboxylate with compound R^{4c} OH (F-IXc) using Mitsunobu conditions, such as DIAD and triphenylphosphine in a solvent such as THF, to provide F-I. Hydrolysis of the ester with aqueous base such as sodium hydroxide or lithium hydroxide in a solvent such as THE leads to carboxylic acids F-II.

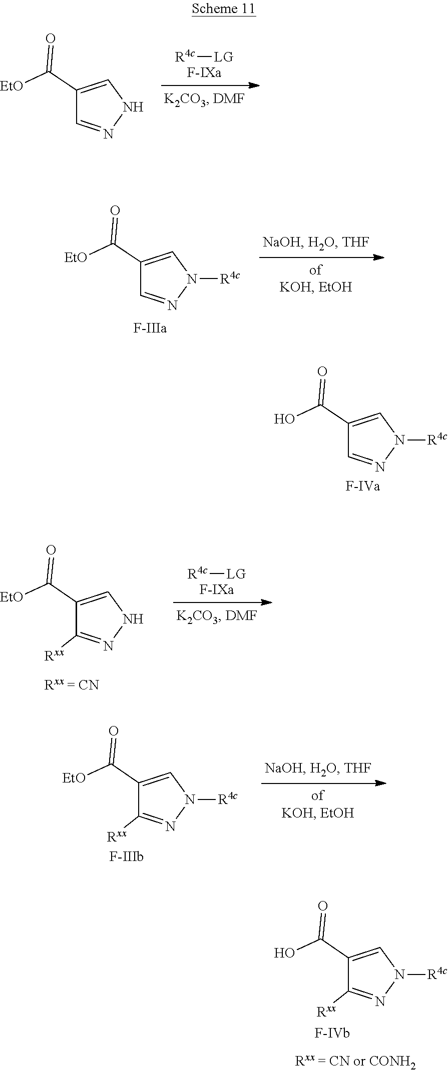

As shown in Scheme 11, substituted 1,2-pyrazole-4-carboxylic acids F-IVa and F-IVb can be prepared in a similar sequence as described in Scheme 10. Saponification of the ester within F-IIIb when R^{xx} is a nitrile using a reagent such as potassium hydroxide in a solvent such as ethanol can lead to the formation of products of general formula F-IVb as a mixture of the corresponding nitrile (R^{xx} is CN) and the primary amide (R^{xx} is CONH₂).

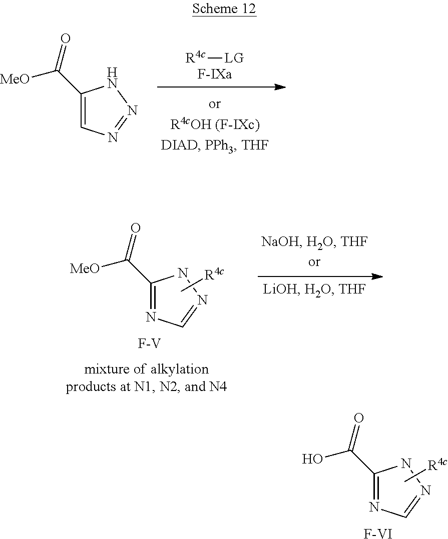

As shown in Scheme 12, substituted 1,2,4-triazole-5-carboxylic acids F-VI can be prepared in a similar sequence as described in Scheme 10 using methyl 1,2,4-triazole-5-carboxylate as the starting material.

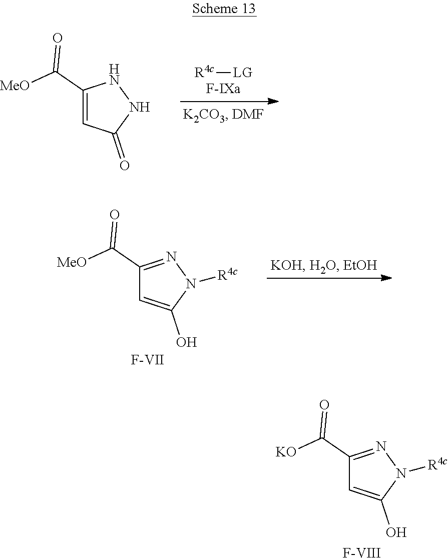

Potassium 5-hydroxy-1H-pyrazole-3-carboxylates F-VIII can be prepared as shown in Scheme 13. Alkylation of methyl 5-oxo-2,5-dihydro-1H-pyrazole-3-carboxylate using a compound of formula F-IXa in the presence of a base such as potassium carbonate in solvents such as DMF affords esters F-VII. Subsequent saponification using regents such as aqueous potassium hydroxide in solvents such as ethanol provides the potassium carboxylate salts of general structure F-VIII.

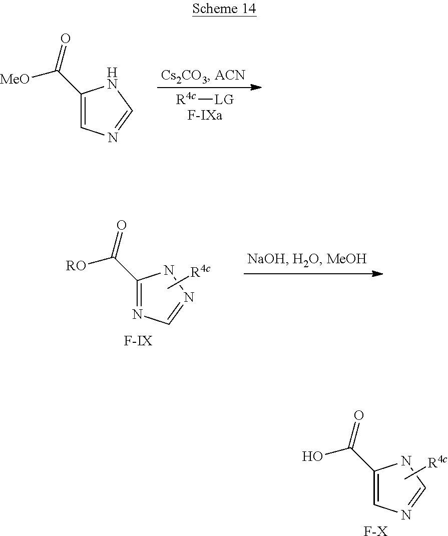

Imidazole-5-carboxylic acids F-X can be prepared as shown in Scheme 14. Alkylation of methyl 1H-imidazole-4-carboxylate using a base such as cesium carbonate and a compound of formula F-IXa in a solvent such as acetonitrile affords alkylated compounds of general formula F-IX. Saponification of the ester using reagents such as sodium hydroxide in a solvent such as methanol affords the corresponding carboxylic acids F-X.

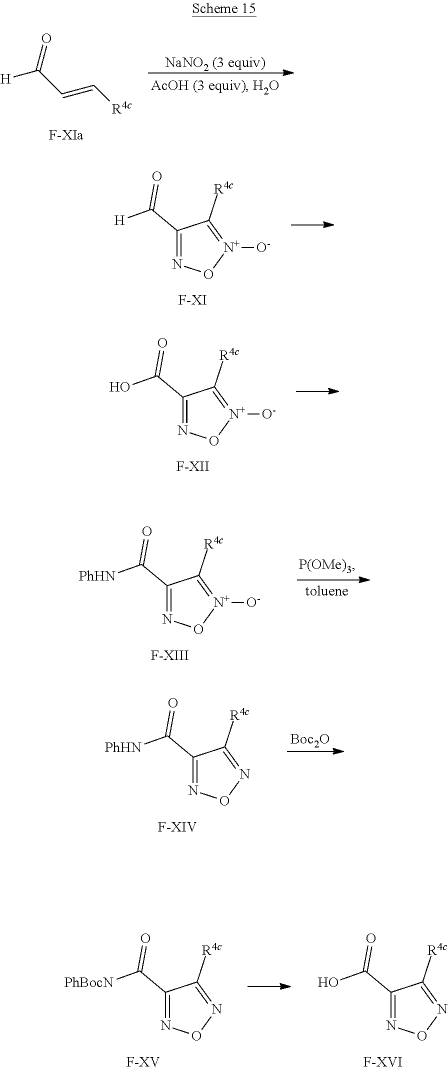

Oxadiazole acids F-XII and F-XVI can be prepared as shown in Scheme 15. Treatment of aldehydes F-XIa with sodium nitrite in solvents such as acetic acid affords the corresponding oxadiazole formyl-1,2,5-oxadiazole 2-oxides F-XI. Oxidation of F-XI with a regent such as Jones reagent in a solvent such as acetone affords carboxylic acids F-XII. Amide bond formation with aniline using reagents such as HATU in solvents such as DMF in the presence of additives such as DIPEA gives amides F-XIII. Reduction of the N-oxide within compounds of formula F-XIII using reagents such as trimethyl phosphite generates oxadiazoles F-XIV. Anilinic amides F-XIV can be treated with a reagent such as di-tert-butyl dicarbonate in a solvent such as DCM in the presence of additives such as DMAP to afford the corresponding carbamates F-XV. Subsequent hydrolysis using reagents such as aqueous LiOH in solvents such as THE then generates carboxylic acids of general formula F-XVI.

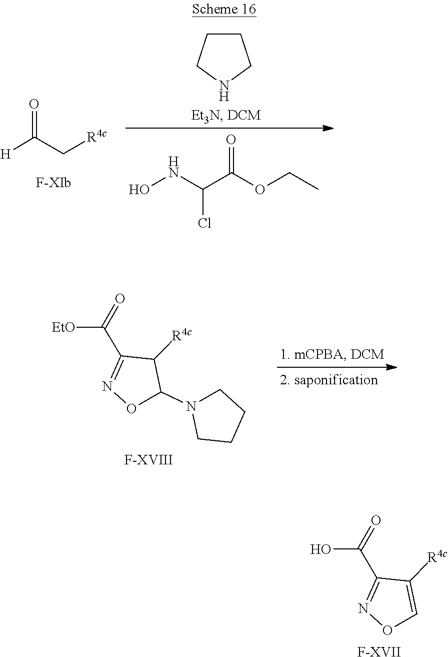

4-Substituted isoxazole-3-carboxylic acids of the general formula F-XVII can be prepared as shown in Scheme 16. Condensation of a suitable aldehyde, F-XIb, with ethyl 2-chloro-2-(hydroxyamino)acetate in the presence of pyrrolidine in a solvent such as DCM in the presence of triethylamine then yields compounds F-XVIII. Oxidation of the pyrrolidine within F-XVIII using reagents such as mCPBA in solvents such as DCM then affords the corresponding isoxazole-3-carboxylic esters (structure not shown) that upon saponification using regents such as aqueous LiOH in solvents such as THE affords compounds of formula F-XVII.

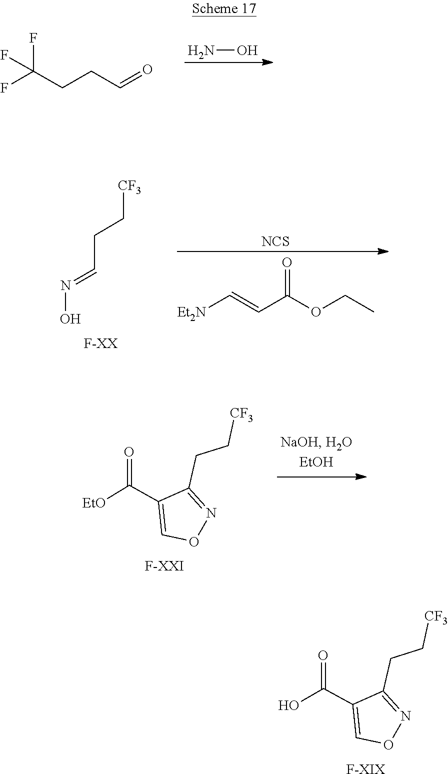

3-Substituted isoxazole 4-carboxylic acid F-XIX is prepared as shown in Scheme 17. Condensation of 4,4,4-trifluorobutanal with hydroxylamine hydrochloride in a solvent such as ethanol yields the corresponding oxime F-XX. Sequential treatment of F-XX with NCS and ethyl-3-(diethylamino)acrylate in a solvent such as chloroform yields ester F-XXI. Saponification of the ester with sodium hydroxide in aqueous ethanol yields isoxazole 4-carboxylic acid F-XIX.

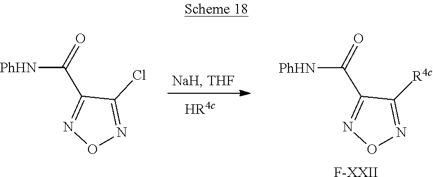

Compounds of general formula F-XXII can be prepared as shown in Scheme 18. 4-Chloro-N-phenyl-1,2,5-oxadiazole-3-carboxamide can be treated with certain alcohols HR^{4c} in the presence of reagents such as sodium hydride in solvents such as THE to afford compounds of general formula F-XXII, wherein R^{4c} is —O—C₍₁₋₃₎alkyl, which is unsubstituted or substituted with one to six substituents independently selected from the group consisting of fluorine, —OH, and —CN.

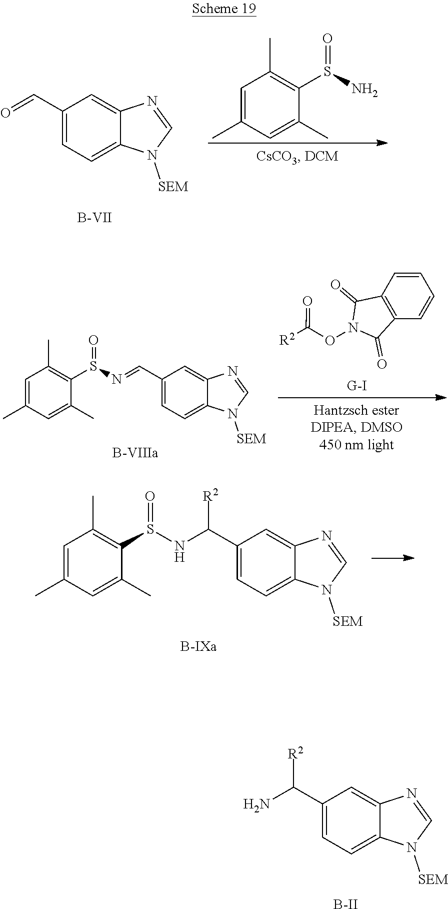

Benzimidazoles B-II can also be prepared as shown in Scheme 19. Condensation of aldehyde B-VII with (R)-2,4,6-trimethylbenzenesulfinamide in the presence of an additive such as Cs₂CO₃ in solvents such as DCM then provides the corresponding sulfinimide B-VIIIa. Reaction of sulfinimide B-VIIIa with a dioxoisoindoline reagent, such as compounds G-I, in the presence of additives such as Hantzsch ester and DIPEA in solvents such as DMSO, with 450 nm light, affords sulfinamides B-IXa. Subsequent deprotection of the sulfinamide group within B-IXa using sulfinamide deprotection conditions provides amines B-II.

### Intermediate 1

### (R)—N—((S)-(5-((R)-1-Aminoethyl)-1H-benzo[d]imidazol-2-yl)(4,4-difluorocyclohexyl)methyl)-2-methylpropane-2-sulfinamide

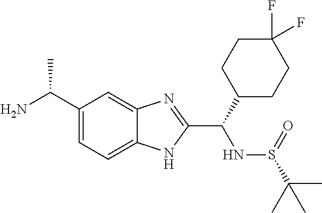 [SMILES: C[C@@H](N)c1ccc2[nH]c([C@@H](N[S@](=O)C(C)(C)C)C3CCC(F)(F)CC3)nc2c1]

### Intermediate 2

### (R)—N—((S)-(5-((S)-1-Aminoethyl)-1H-benzo[d]imidazol-2-yl)(4,4-difluorocyclohexyl)methyl)-2-methylpropane-2-sulfinamide

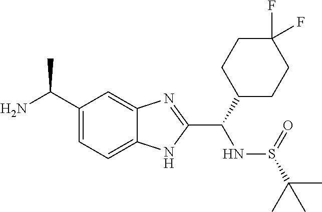 [SMILES: C[C@H](N)c1ccc2[nH]c([C@@H](N[S@](=O)C(C)(C)C)C3CCC(F)(F)CC3)nc2c1]

MeMgBr (634 mL, 1.9 mol, 3 M in Et₂O) was added dropwise to a 0° C. solution of (R)—N—((S)-(5-cyano-1H-benzo[d]imidazol-2-yl)(4,4-difluorocyclohexyl)methyl)-2-methylpropane-2-sulfinamide (75 g, 190 mmol, Intermediate 62) in THF (750 mL) and the resulting solution was stirred at 60° C. for 17 h. The solution was then cooled to 0° C. and a saturated solution of NH₃ in MeOH (750 mL) was added dropwise. The solution was stirred at 0° C. for 15 min, then NaBH₄ (7.19 g, 190 mmol) was added and the solution was stirred at 15° C. for 2 h. The reaction was quenched with water (2 L) and extracted with a mixture of DCM (2 L) and MeOH (600 mL). The organic layer was washed with brine (1 L), dried over anhydrous MgSO₄, filtered and concentrated to dryness. The crude material was purified by silica gel chromatography (0-9% MeOH/DCM) to provide the title compounds, a mixture of diastereomers, as a yellow solid. The diastereomers were separated by SFC using a chiral stationary phase (DAICEL CHIRALPAK AD, 10 μm, 250×50 mm, mobile phase: 40% CO₂ in EtOH (0.1% NH₄OH)). The first eluting isomer was Intermediate 1 and the second eluting isomer was Intermediate 2.

### Intermediate 3

### N—((R)-1-(2-((S)—(((R)-tert-Butylsulfinyl)amino)(4,4-difluorocyclohexyl)methyl)-1H-benzo[d]imidazol-5-yl)ethyl)-4,4,4-trifluorobutanamide

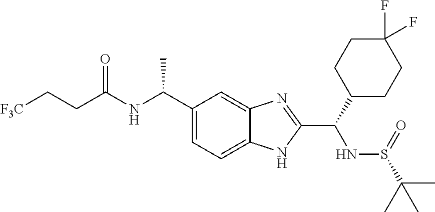 [SMILES: C[C@@H](NC(=O)CCC(F)(F)F)c1ccc2[nH]c([C@@H](N[S@](=O)C(C)(C)C)C3CCC(F)(F)CC3)nc2c1]

To a mixture of (R)—N—((S)-(5-((R)-1-aminoethyl)-1H-benzo[d]imidazol-2-yl)(4,4-difluorocyclohexyl)methyl)-2-methylpropane-2-sulfinamide (2.0 g, 4.9 mmol, Intermediate 1), 4,4,4-trifluorobutyric acid (808 mg, 5.58 mmol), HOBt (688 mg, 5.09 mmol), DIPEA (1 mL, 5.8 mmol) and ACN (54 mL) was added EDCI (976 mg, 5.09 mmol). The resulting mixture was stirred at rt for 2 h. The reaction was quenched by the addition of water and then extracted with EtOAc (2×50 mL). The organic layers were combined, washed with brine (50 mL), dried over anhydrous Na₂SO₄, filtered, and concentrated to dryness. The residue was purified by silica gel chromatography (0-100% DCM (with 10% 2 M NH₃ in MeOH)/DCM) to provide the title compound as a white foam.

### Intermediate 4

### N—((R)-1-(2-((S)-Amino(4,4-difluorocyclohexyl)methyl)-1H-benzo[d]imidazol-5-yl)ethyl)-4,4,4-trifluorobutanamide

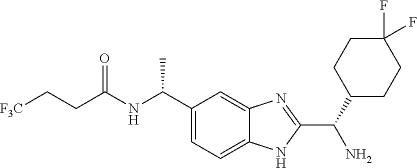 [SMILES: C[C@@H](NC(=O)CCC(F)(F)F)c1ccc2[nH]c([C@@H](N)C3CCC(F)(F)CC3)nc2c1]

To a solution of N—((R)-1-(2-((S)—(((R)-tert-butylsulfinyl)amino)(4,4-difluorocyclohexyl)methyl)-1H-benzo[d]imidazol-5-yl)ethyl)-4,4,4-trifluorobutanamide (1.94 g, 3.61 mmol, Intermediate 3) in EtOAc (7.1 mL) was added a solution of HCl in 1,4-dioxane (2.7 mL, 10.8 mmol, 4 M) and the resulting mixture was stirred at rt for 2 h. The reaction mixture was concentrated to dryness and the residue dissolved in water. The pH of the mixture was adjusted to ˜pH 8 by the addition of 1 N aqueous NaOH and then the mixture was extracted with DCM (2×15 mL). The organic layers were combined, dried over anhydrous Na₂SO₄, filtered, and concentrated to dryness. The residue was purified by silica gel chromatography (0-100% DCM (with 10% 2 M NH₃ in MeOH)/DCM) to provide the title compound as a white foam.

### Intermediate 5

### Methyl 2-(3,3,3-trifluoropropyl)-2H-1,2,3-triazole-4-carboxylate

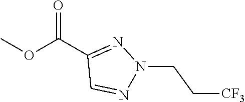

To a mixture of methyl 1H-1,2,3-triazole-4-carboxylate (5 g, 38.2 mmol), K₂CO₃ (5.27 g, 38.2 mmol) and DMF (49 mL) was added 3-bromo-1,1,1-trifluoropropane (4.07 mL, 38.2 mmol) and the resulting mixture was stirred at rt for 17 h. The mixture was filtered through a pad of Celite®, rinsed with EtOAc and the filtrate concentrated under vacuum. The residue was partitioned between EtOAc (50 mL) and water (50 mL). The layers were separated and the aqueous layer was further extracted with EtOAc (2×50 mL). The organic layers were combined, washed with brine (50 mL), dried over anhydrous Na₂SO₄, filtered and concentrated to dryness. The residue was purified by silica gel chromatography (0-75% EtOAc/hexanes; second eluting isomer) to provide the title compound as a white solid.

### Intermediate 6

### Methyl 1-(3,3,3-trifluoropropyl)-1H-1,2,3-triazole-5-carboxylate

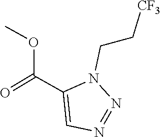

The title compound was prepared as described for the synthesis of Intermediate 5. Methyl 1-(3,3,3-trifluoropropyl)-1H-1,2,3-triazole-5-carboxylate was the first eluting isomer, isolated as a clear colorless oil.

### Intermediate 7

### 2-(3,3,3-Trifluoropropyl)-2H-1,2,3-triazole-4-carboxylic acid

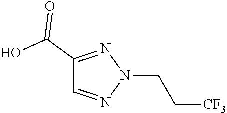 [SMILES: O=C(O)c1cnn(CCC(F)(F)F)n1]

To a mixture of methyl 2-(3,3,3-trifluoropropyl)-2H-1,2,3-triazole-4-carboxylate (4.28 g, 19.2 mmol, Intermediate 5) in THF (58 mL) was added 2 M aqueous NaOH (58 mL, 115 mmol) and the mixture was stirred at rt for 15 h. After that time, the mixture was concentrated to remove the THE and then washed with EtOAc (2×50 mL). The aqueous layer was then acidified to pH 3 by the addition of 1 N aqueous HCl and extracted with 2-MeTHF (3×50 mL). The organic layers were combined, washed with brine (50 mL), dried over anhydrous Na₂SO₄, filtered, and concentrated to dryness to provide the title compound as a white solid.

### Intermediate 8

### 1-(3,3,3-Trifluoropropyl)-1H-1,2,3-triazole-5-carboxylic acid

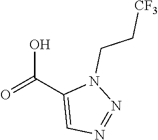 [SMILES: O=C(O)c1cnnn1CCC(F)(F)F]

The title compound was prepared as described for the synthesis of Intermediate 7, using methyl 1-(3,3,3-trifluoropropyl)-1H-1,2,3-triazole-5-carboxylate (Intermediate 6) in place of methyl 2-(3,3,3-trifluoropropyl)-2H-1,2,3-triazole-4-carboxylate, to provide the title compound as a white solid.

### Intermediate 9

### Methyl 2-(cyclopropylmethyl)-2H-1,2,3-triazole-4-carboxylate

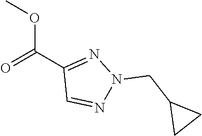

The title compound was prepared as described for the synthesis of Intermediate 5, using (bromomethyl)cyclopropane in place of 3-bromo-1,1,1-trifluoropropane. The first eluting isomer was isolated to provide the title compound as a clear colorless oil.

### Intermediate 10

### 2-(Cyclopropylmethyl)-2H-1,2,3-triazole-4-carboxylic acid

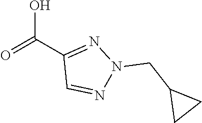 [SMILES: O=C(O)c1cnn(CC2CC2)n1]

The title compound was prepared as described for the synthesis of Intermediate 7, using methyl 2-(cyclopropylmethyl)-2H-1,2,3-triazole-4-carboxylate (Intermediate 9) in place of methyl 2-(3,3,3-trifluoropropyl)-2H-1,2,3-triazole-4-carboxylate, to provide the title compound as a white solid.

### Intermediate 11

### Methyl 1-(cyclopropylmethyl)-1H-1,2,3-triazole-5-carboxylate

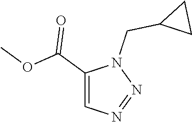

The title compound was prepared as described for the synthesis of Intermediate 9. Methyl 1-(cyclopropylmethyl)-1H-1,2,3-triazole-5-carboxylate was the second eluting isomer, isolated as a yellow oil.

### Intermediate 12

### 1-(Cyclopropylmethyl)-1H-1,2,3-triazole-5-carboxylic acid

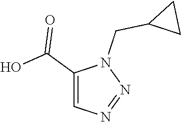 [SMILES: O=C(O)c1cnnn1CC1CC1]

The title compound was prepared as described for the synthesis of Intermediate 7, using methyl 1-(cyclopropylmethyl)-1H-1,2,3-triazole-5-carboxylate (Intermediate 11) in place of methyl 2-(3,3,3-trifluoropropyl)-2H-1,2,3-triazole-4-carboxylate, to provide the title compound as a white solid.

### Intermediate 13

### Methyl 1-(cyclopropylmethyl)-1H-1,2,3-triazole-4-carboxylate

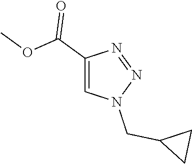

The title compound was prepared as described for the synthesis of Intermediate 9. Methyl 1-(cyclopropylmethyl)-1H-1,2,3-triazole-4-carboxylate was the third eluting isomer, isolated as a white solid.

### Intermediate 14

### 1-(Cyclopropylmethyl)-1H-1,2,3-triazole-4-carboxylic acid

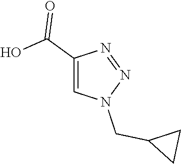 [SMILES: O=C(O)c1cn(CC2CC2)nn1]

The title compound was prepared as described for the synthesis of Intermediate 7, using methyl 1-(cyclopropylmethyl)-1H-1,2,3-triazole-4-carboxylate (Intermediate 13) in place of methyl 2-(3,3,3-trifluoropropyl)-2H-1,2,3-triazole-4-carboxylate, to provide the title compound as a white solid.

### Intermediate 15

### Ethyl 2-(2-cyclopropylethyl)-2H-1,2,3-triazole-4-carboxylate

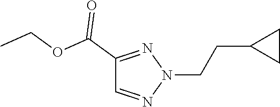

To a mixture of ethyl 1H-1,2,3-triazole-4-carboxylate (3 g, 20.2 mmol), 2-cyclopropylethanol (2.79 g, 30.3 mmol), PPh₃ (5.77 g, 22 mmol) and THF (67.3 mL) at 0° C. was added DIAD (4.3 mL, 22 mmol) over 10 min and the resulting mixture stirred at rt for 2.5 h. The reaction mixture was concentrated to dryness and the residue purified by silica gel chromatography (0-75% EtOAc/hexanes). The first eluting isomer was isolated to provide the title compound as a clear colorless oil.

### Intermediate 16

### 2-(2-Cyclopropylethyl)-2H-1,2,3-triazole-4-carboxylic acid

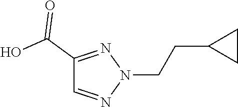 [SMILES: O=C(O)c1cnn(CCC2CC2)n1]

The title compound was prepared as described for the synthesis of Intermediate 7, using ethyl 2-(2-cyclopropylethyl)-2H-1,2,3-triazole-4-carboxylate (Intermediate 15) in place of methyl 2-(3,3,3-trifluoropropyl)-2H-1,2,3-triazole-4-carboxylate, to provide the title compound as a white solid.

### Intermediate 17

### Ethyl 1-(2-cyclopropylethyl)-1H-1,2,3-triazole-5-carboxylate

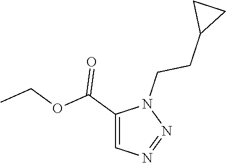

The title compound was prepared as described for the synthesis of Intermediate 15. Ethyl 1-(2-cyclopropylethyl)-1H-1,2,3-triazole-5-carboxylate was the second eluting isomer, isolated as a light-yellow oil.

### Intermediate 18

### 1-(2-Cyclopropylethyl)-1H-1,2,3-triazole-5-carboxylic acid

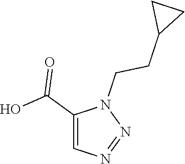 [SMILES: O=C(O)c1cnnn1CCC1CC1]

The title compound was prepared as described for the synthesis of Intermediate 7, using ethyl 1-(2-cyclopropylethyl)-1H-1,2,3-triazole-5-carboxylate (Intermediate 17) in place of methyl 2-(3,3,3-trifluoropropyl)-2H-1,2,3-triazole-4-carboxylate, to provide the title compound as a white solid.

### Intermediate 19

### Ethyl 2-(2-methoxyethyl)-2H-1,2,3-triazole-4-carboxylate

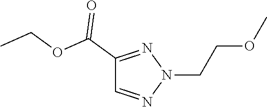

The title compound was prepared as described for the synthesis of Intermediate 5, using ethyl 1H-1,2,3-triazole-4-carboxylate in place of methyl 1H-1,2,3-triazole-4-carboxylate and 2-bromoethyl methyl ether in place of 3-bromo-1,1,1-trifluoropropane. The first eluting isomer was isolated to provide the title compound as a yellow oil.

### Intermediate 20

### 2-(2-Methoxyethyl)-2H-1,2,3-triazole-4-carboxylic acid

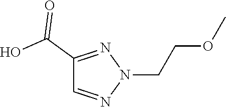

The title compound was prepared as described for the synthesis of Intermediate 7, using ethyl 2-(2-methoxyethyl)-2H-1,2,3-triazole-4-carboxylate (Intermediate 19) in place of methyl 2-(3,3,3-trifluoropropyl)-2H-1,2,3-triazole-4-carboxylate, to provide the title compound as a yellow solid.

### Intermediate 21

### Ethyl 1-(2-methoxyethyl)-1H-1,2,3-triazole-5-carboxylate

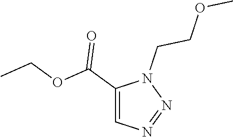

The title compound was prepared as described for the synthesis of Intermediate 19. Ethyl 1-(2-methoxyethyl)-1H-1,2,3-triazole-5-carboxylate was the second eluting isomer, isolated as a yellow oil.

### Intermediate 22

### 1-(2-Methoxyethyl)-1H-1,2,3-triazole-5-carboxylic acid

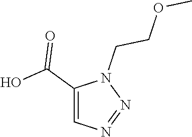

The title compound was prepared as described for the synthesis of Intermediate 7, using ethyl 1-(2-methoxyethyl)-1H-1,2,3-triazole-5-carboxylate (Intermediate 21) in place of methyl 2-(3,3,3-trifluoropropyl)-2H-1,2,3-triazole-4-carboxylate, to provide the title compound as a yellow solid.

### Intermediate 23

### Ethyl 1-(2-methoxyethyl)-1H-1,2,3-triazole-4-carboxylate

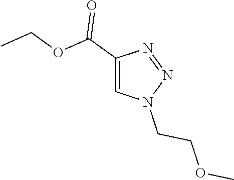

The title compound was prepared as described for the synthesis of Intermediate 19. Ethyl 1-(2-methoxyethyl)-1H-1,2,3-triazole-4-carboxylate the third eluting isomer, isolated as a yellow oil.

### Intermediate 24

### 1-(2-Methoxyethyl)-1H-1,2,3-triazole-4-carboxylic acid

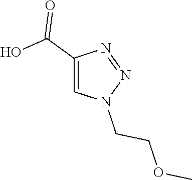

The title compound was prepared as described for the synthesis of Intermediate 7, using ethyl 1-(2-methoxyethyl)-1H-1,2,3-triazole-4-carboxylate (Intermediate 23) in place of methyl 2-(3,3,3-trifluoropropyl)-2H-1,2,3-triazole-4-carboxylate, to provide the title compound as a cream-colored solid.

### Intermediate 25

### Ethyl 2-(4,4,4-trifluorobutyl)-2H-1,2,3-triazole-4-carboxylate

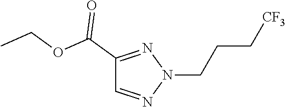 [SMILES: CCOC(=O)c1cnn(CCCC(F)(F)F)n1]

The title compound was prepared as described for the synthesis of Intermediate 5, using ethyl 1H-1,2,3-triazole-4-carboxylate in place of methyl 1H-1,2,3-triazole-4-carboxylate and 1-bromo-4,4,4-trifluorobutane in place of 3-bromo-1,1,1-trifluoropropane. The first eluting isomer was isolated to provide the title compound as a yellow oil.

### Intermediate 26

### 2-(4,4,4-Trifluorobutyl)-2H-1,2,3-triazole-4-carboxylic acid

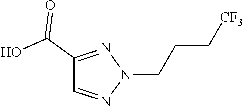 [SMILES: O=C(O)c1cnn(CCCC(F)(F)F)n1]

The title compound was prepared as described for the synthesis of Intermediate 7, using ethyl 2-(4,4,4-trifluorobutyl)-2H-1,2,3-triazole-4-carboxylate (Intermediate 25) in place of methyl 2-(3,3,3-trifluoropropyl)-2H-1,2,3-triazole-4-carboxylate, to provide the title compound as a white solid.

### Intermediate 27

### Ethyl 1-(4,4,4-trifluorobutyl)-1H-1,2,3-triazole-5-carboxylate

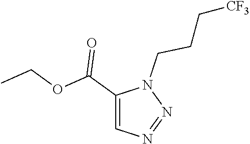 [SMILES: CCOC(=O)c1cnnn1CCCC(F)(F)F]

The title compound was prepared as described for the synthesis of Intermediate 25. Ethyl 1-(4,4,4-trifluorobutyl)-1H-1,2,3-triazole-5-carboxylate was the second eluting isomer, isolated as a yellow oil.

### Intermediate 28

### 1-(4,4,4-Trifluorobutyl)-1H-1,2,3-triazole-5-carboxylic acid

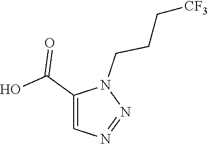 [SMILES: O=C(O)c1cnnn1CCCC(F)(F)F]

The title compound was prepared as described for the synthesis of Intermediate 7, using ethyl 1-(4,4,4-trifluorobutyl)-1H-1,2,3-triazole-5-carboxylate (Intermediate 27) in place of methyl 2-(3,3,3-trifluoropropyl)-2H-1,2,3-triazole-4-carboxylate, to provide the title compound as a white solid.

### Intermediate 29

### Ethyl 1-(4,4,4-trifluorobutyl)-1H-1,2,3-triazole-4-carboxylate

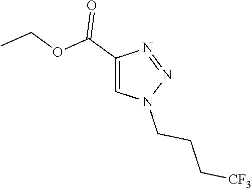 [SMILES: CCOC(=O)c1cn(CCCC(F)(F)F)nn1]

The title compound was prepared as described for the synthesis of Intermediate 25. Ethyl 1-(4,4,4-trifluorobutyl)-1H-1,2,3-triazole-4-carboxylate was the third eluting isomer, isolated as a yellow solid.

### Intermediate 30

### 1-(4,4,4-Trifluorobutyl)-1H-1,2,3-triazole-4-carboxylic acid

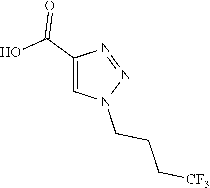 [SMILES: O=C(O)c1cn(CCCC(F)(F)F)nn1]

The title compound was prepared as described for the synthesis of Intermediate 7, using ethyl 1-(4,4,4-trifluorobutyl)-1H-1,2,3-triazole-4-carboxylate (Intermediate 29) in place of methyl 2-(3,3,3-trifluoropropyl)-2H-1,2,3-triazole-4-carboxylate, to provide the title compound as a white solid.

### Intermediate 31

### Ethyl 2-(2,2,2-trifluoroethyl)-2H-1,2,3-triazole-4-carboxylate

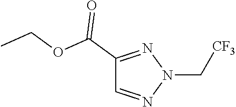

To a mixture of ethyl 1H-1,2,3-triazole-4-carboxylate (1 g, 6.7 mmol), Cs₂CO₃ (2.19 g, 6.73 mmol), and DMF (8.6 mL) was added 2-iodo-1,1,1-trifluoroethane (0.67 mL, 6.7 mmol). The resulting mixture was stirred at 40° C. for 2.5 h. An additional aliquot of 2-iodo-1,1,1-trifluoroethane (0.67 mL, 6.7 mmol) was added and the mixture stirred at 60° C. for 23 h followed by 80° C. for 3 d. After that time the mixture was filtered through a pad of Celite®, rinsed with EtOAc, and the filtrate concentrated under vacuum. The residue was partitioned between EtOAc (30 mL) and water (30 mL). The layers were separated, and the aqueous layer was further extracted with EtOAc (2×30 mL). The organic layers were combined, washed with brine (50 mL), dried over anhydrous Na₂SO₄, filtered, and concentrated to dryness. The residue was purified by silica gel chromatography (0-75% EtOAc/hexanes) and the first eluting isomer was isolated to provide the title compound as a clear colorless oil.

### Intermediate 32

### 2-(2,2,2-Trifluoroethyl)-2H-1,2,3-triazole-4-carboxylic acid

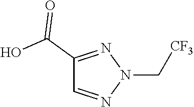 [SMILES: O=C(O)c1cnn(CC(F)(F)F)n1]

The title compound was prepared as described for the synthesis of Intermediate 7, using ethyl 2-(2,2,2-trifluoroethyl)-2H-1,2,3-triazole-4-carboxylate (Intermediate 31) in place of methyl 2-(3,3,3-trifluoropropyl)-2H-1,2,3-triazole-4-carboxylate, to provide the title compound as a white solid.

### Intermediate 33

### Ethyl 1-(2,2,2-trifluoroethyl)-1H-1,2,3-triazole-5-carboxylate

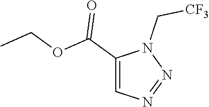

The title compound was prepared as described for the synthesis of Intermediate 31. Ethyl 1-(2,2,2-trifluoroethyl)-1H-1,2,3-triazole-5-carboxylate was the second eluting isomer, isolated as a light-yellow oil.

### Intermediate 34

### 1-(2,2,2-Trifluoroethyl)-1H-1,2,3-triazole-5-carboxylic acid

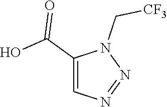 [SMILES: O=C(O)c1cnnn1CC(F)(F)F]

The title compound was prepared as described for the synthesis of Intermediate 7, using ethyl 1-(2,2,2-trifluoroethyl)-1H-1,2,3-triazole-5-carboxylate (Intermediate 33) in place of methyl 2-(3,3,3-trifluoropropyl)-2H-1,2,3-triazole-4-carboxylate, to provide the title compound as a light-yellow solid.

### Intermediate 35

### Ethyl 1-(2,2,2-trifluoroethyl)-1H-1,2,3-triazole-4-carboxylate

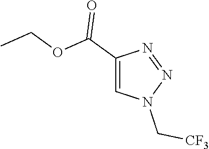

The title compound was prepared as described for the synthesis of Intermediate 31. Ethyl 1-(2,2,2-trifluoroethyl)-1H-1,2,3-triazole-4-carboxylate was the third eluting isomer, isolated as a cream-colored solid.

### Intermediate 36

### 1-(2,2,2-Trifluoroethyl)-1H-1,2,3-triazole-4-carboxylic acid

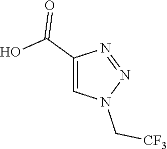 [SMILES: O=C(O)c1cn(CC(F)(F)F)nn1]

The title compound was prepared as described for the synthesis of Intermediate 7, using ethyl 1-(2,2,2-trifluoroethyl)-1H-1,2,3-triazole-4-carboxylate (Intermediate 35) in place of methyl 2-(3,3,3-trifluoropropyl)-2H-1,2,3-triazole-4-carboxylate, to provide the title compound as a light-yellow solid.

### Intermediate 37

### Ethyl 1-(2-(trifluoromethoxy)ethyl)-1H-pyrazole-4-carboxylate

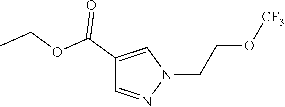 [SMILES: CCOC(=O)c1cnn(CCOC(F)(F)F)c1]

The title compound was prepared as described for the synthesis of Intermediate 5, using ethyl 1H-pyrazole-4-carboxylate in place of methyl 1H-1,2,3-triazole-4-carboxylate and 1-bromo-2-(trifluoromethoxy)ethane in place of 3-bromo-1,1,1-trifluoropropane, to provide the title compound as a white solid.

### Intermediate 38

### 1-(2-(Trifluoromethoxy)ethyl)-1H-pyrazole-4-carboxylic acid

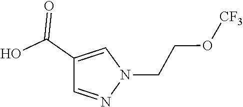 [SMILES: O=C(O)c1cnn(CCOC(F)(F)F)c1]

The title compound was prepared as described for the synthesis of Intermediate 7, using ethyl 1-(2-(trifluoromethoxy)ethyl)-1H-pyrazole-4-carboxylate (Intermediate 37) in place of methyl 2-(3,3,3-trifluoropropyl)-2H-1,2,3-triazole-4-carboxylate. After stirring at rt, the mixture was stirred at 90° C. for 7 h to provide the title compound as a white solid.

### Intermediate 39

### Ethyl 1-(2-(difluoromethoxy)ethyl)-1H-pyrazole-4-carboxylate

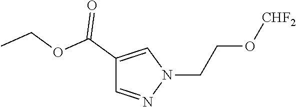 [SMILES: CCOC(=O)c1cnn(CCOC(F)F)c1]

The title compound was prepared as described for the synthesis of Intermediate 5, using ethyl 1H-pyrazole-4-carboxylate in place of methyl 1H-1,2,3-triazole-4-carboxylate and 1-bromo-2-(difluoromethoxy)ethane in place of 3-bromo-1,1,1-trifluoropropane, to provide the title compound as a white solid.

### Intermediate 40

### 1-(2-(Difluoromethoxy)ethyl)-1H-pyrazole-4-carboxylic acid

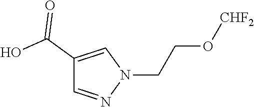 [SMILES: O=C(O)c1cnn(CCOC(F)F)c1]

The title compound was prepared as described for the synthesis of Intermediate 7, using ethyl 1-(2-(difluoromethoxy)ethyl)-1H-pyrazole-4-carboxylate (Intermediate 39) in place of methyl 2-(3,3,3-trifluoropropyl)-2H-1,2,3-triazole-4-carboxylate. After stirring at rt, the mixture was stirred at 90° C. for 3 h to provide the title compound as a white solid.

### Intermediate 41

### Methyl 1-(cyclopropylmethyl)-1H-1,2,4-triazole-3-carboxylate

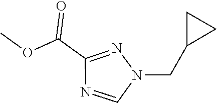

The title compound was prepared as described for the synthesis of Intermediate 5, using methyl-1H-1,2,4-triazole-3-carboxylate in place of methyl 1H-1,2,3-triazole-4-carboxylate and (bromomethyl)cyclopropane in place of 3-bromo-1,1,1-trifluoropropane. Methyl 1-(cyclopropylmethyl)-1H-1,2,4-triazole-3-carboxylate was the second eluting isomer, isolated as a white solid.

### Intermediate 42

### 1-(Cyclopropylmethyl)-1H-1,2,4-triazole-3-carboxylic acid

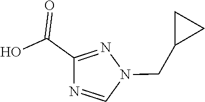 [SMILES: O=C(O)c1ncn(CC2CC2)n1]

The title compound was prepared as described for the synthesis of Intermediate 7, using methyl 1-(cyclopropylmethyl)-1H-1,2,4-triazole-3-carboxylate (Intermediate 41) in place of methyl 2-(3,3,3-trifluoropropyl)-2H-1,2,3-triazole-4-carboxylate, to provide the title compound as a white solid.

### Intermediate 43

### Methyl 1-(cyclopropylmethyl)-1H-1,2,4-triazole-5-carboxylate

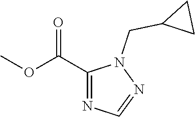

The title compound was prepared as described for the synthesis of Intermediate 41. Methyl 1-(cyclopropylmethyl)-1H-1,2,4-triazole-5-carboxylate was the first eluting isomer, isolated as a clear colorless oil.

### Intermediate 44

### 1-(Cyclopropylmethyl)-1H-1,2,4-triazole-5-carboxylic acid

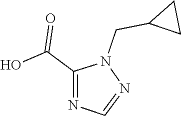 [SMILES: O=C(O)c1ncnn1CC1CC1]

The title compound was prepared as described for the synthesis of Intermediate 7, using methyl 1-(cyclopropylmethyl)-1H-1,2,4-triazole-5-carboxylate (Intermediate 43) in place of methyl 2-(3,3,3-trifluoropropyl)-2H-1,2,3-triazole-4-carboxylate, to provide the title compound as a white solid.

### Intermediate 45

### Ethyl 2-(2-(difluoromethoxy)ethyl)-2H-1,2,3-triazole-4-carboxylate

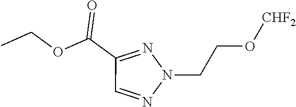

The title compound was prepared as described for the synthesis of Intermediate 5, using ethyl 1H-1,2,3-triazole-4-carboxylate in place of methyl 1H-1,2,3-triazole-4-carboxylate and 1-bromo-2-(difluoromethoxy)ethane in place of 3-bromo-1,1,1-trifluoropropane. Ethyl 2-(2-(difluoromethoxy)ethyl)-2H-1,2,3-triazole-4-carboxylate was the first eluting isomer, isolated as a yellow oil.

### Intermediate 46

### 2-(2-(Difluoromethoxy)ethyl)-2H-1,2,3-triazole-4-carboxylic acid

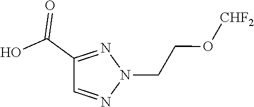

The title compound was prepared as described for the synthesis of Intermediate 7, using ethyl 2-(2-(difluoromethoxy)ethyl)-2H-1,2,3-triazole-4-carboxylate (Intermediate 45) in place of methyl 2-(3,3,3-trifluoropropyl)-2H-1,2,3-triazole-4-carboxylate, to provide the title compound as a white solid.

### Intermediate 47

### Ethyl 1-(2-(difluoromethoxy)ethyl)-1H-1,2,3-triazole-5-carboxylate

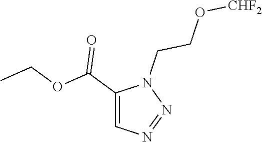 [SMILES: CCOC(=O)c1cnnn1CCOC(F)F]

The title compound was prepared as described for the synthesis of Intermediate 45. Ethyl 1-(2-(difluoromethoxy)ethyl)-1H-1,2,3-triazole-5-carboxylate was the second eluting isomer, isolated as a yellow oil.

### Intermediate 48

### 1-(2-(Difluoromethoxy)ethyl)-1H-1,2,3-triazole-5-carboxylic acid

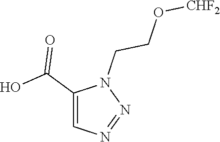 [SMILES: O=C(O)c1cnnn1CCOC(F)F]

The title compound was prepared as described for the synthesis of Intermediate 7, using ethyl 1-(2-(difluoromethoxy)ethyl)-1H-1,2,3-triazole-5-carboxylate (Intermediate 47) in place of methyl 2-(3,3,3-trifluoropropyl)-2H-1,2,3-triazole-4-carboxylate, to provide the title compound as a white solid.

### Intermediate 49

### Ethyl 2-(2-(trifluoromethoxy)ethyl)-2H-1,2,3-triazole-4-carboxylate

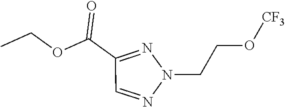 [SMILES: CCOC(=O)c1cnn(CCOC(F)(F)F)n1]

The title compound was prepared as described for the synthesis of Intermediate 5, using ethyl 1H-1,2,3-triazole-4-carboxylate in place of methyl 1H-1,2,3-triazole-4-carboxylate and 1-bromo-2-(trifluoromethoxy)ethane in place of 3-bromo-1,1,1-trifluoropropane. Ethyl 2-(2-(trifluoromethoxy)ethyl)-2H-1,2,3-triazole-4-carboxylate was the first eluting isomer, isolated as a white solid.

### Intermediate 50

### 2-(2-(Trifluoromethoxy)ethyl)-2H-1,2,3-triazole-4-carboxylic acid

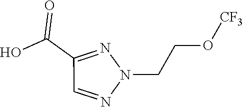 [SMILES: O=C(O)c1cnn(CCOC(F)(F)F)n1]

The title compound was prepared as described for the synthesis of Intermediate 7, using ethyl 2-(2-(trifluoromethoxy)ethyl)-2H-1,2,3-triazole-4-carboxylate (Intermediate 49) in place of methyl 2-(3,3,3-trifluoropropyl)-2H-1,2,3-triazole-4-carboxylate, to provide the title compound as a white solid.

### Intermediate 51

### Ethyl 1-(2-(trifluoromethoxy)ethyl)-1H-1,2,3-triazole-5-carboxylate

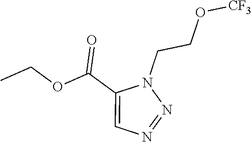

The title compound was prepared as described for the synthesis of Intermediate 49. Ethyl 1-(2-(trifluoromethoxy)ethyl)-1H-1,2,3-triazole-5-carboxylate was the second eluting isomer, isolated as a white solid.

### Intermediate 52

### 1-(2-(Trifluoromethoxy)ethyl)-1H-1,2,3-triazole-5-carboxylic acid

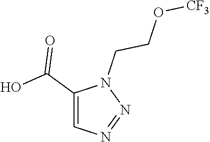 [SMILES: O=C(O)c1cnnn1CCOC(F)(F)F]

The title compound was prepared as described for the synthesis of Intermediate 7, using ethyl 1-(2-(trifluoromethoxy)ethyl)-1H-1,2,3-triazole-5-carboxylate (Intermediate 51) in place of methyl 2-(3,3,3-trifluoropropyl)-2H-1,2,3-triazole-4-carboxylate, to provide the title compound as a white solid.

### Intermediate 53

### Ethyl 2-(2,2-difluoroethyl)-2H-1,2,3-triazole-4-carboxylate

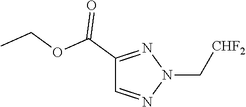

The title compound was prepared as described for the synthesis of Intermediate 5, using ethyl 1H-1,2,3-triazole-4-carboxylate in place of methyl 1H-1,2,3-triazole-4-carboxylate and 2-bromo-1,1-difluoroethane in place of 3-bromo-1,1,1-trifluoropropane. After stirring at rt for 17 h, an additional aliquot of 2-bromo-1,1-difluoroethane (0.83 mL, 10.2 mmol) was added and the mixture stirred at rt for 3 d. The first eluting isomer was isolated to provide the title compound as a clear colorless oil.

### Intermediate 54

### 2-(2,2-Difluoroethyl)-2H-1,2,3-triazole-4-carboxylic acid

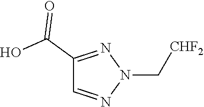 [SMILES: O=C(O)c1cnn(CC(F)F)n1]

The title compound was prepared as described for the synthesis of Intermediate 7, using ethyl 2-(2,2-difluoroethyl)-2H-1,2,3-triazole-4-carboxylate (Intermediate 53) in place of methyl 2-(3,3,3-trifluoropropyl)-2H-1,2,3-triazole-4-carboxylate, and was stirred at 45° C. for 2 h after stirring at rt. The crude material was purified by acidic preparative HPLC to provide the title compound as a cream-colored solid.

### Intermediate 55

### Ethyl 1-(2,2-difluoroethyl-1H-1,2,3-triazole-5-carboxylate

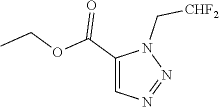

The title compound was prepared as described for the synthesis of Intermediate 53. Ethyl 1-(2,2-difluoroethyl)-1H-1,2,3-triazole-5-carboxylate was the second eluting isomer, isolated as a yellow oil.

### Intermediate 56

### 1-(2,2-Difluoroethyl)-1H-1,2,3-triazole-5-carboxylic acid

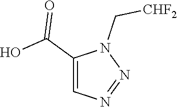 [SMILES: O=C(O)c1cnnn1CC(F)F]

The title compound was prepared as described for the synthesis of Intermediate 7, using ethyl 1-(2,2-difluoroethyl)-1H-1,2,3-triazole-5-carboxylate (Intermediate 55) in place of methyl 2-(3,3,3-trifluoropropyl)-2H-1,2,3-triazole-4-carboxylate, to provide the title compound as a white solid.

### Intermediate 57

### Methyl 1-(3,3-difluoropropyl)-1H-1,2,4-triazole-3-carboxylate

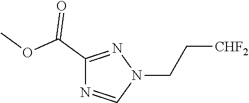

The title compound was prepared as described for the synthesis of Intermediate 15, using methyl 1H-1,2,4-triazole-3-carboxylate in place of ethyl 1H-1,2,3-triazole-4-carboxylate and 3,3-difluoropropan-1-ol in place of 2-cyclopropylethanol. In addition, the reagents were combined at rt instead of at 0° C. followed by stirring at rt for 1 h instead of 2.5 h. Methyl 1-(3,3-difluoropropyl)-1H-1,2,4-triazole-3-carboxylate was the second eluting isomer, isolated as a white amorphous solid.

### Intermediate 58

### 1-(3,3-Difluoropropyl)-1H-1,2,4-triazole-3-carboxylic acid

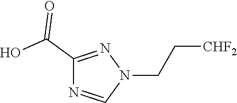 [SMILES: O=C(O)c1ncn(CCC(F)F)n1]

The title compound was prepared as described for the synthesis of Intermediate 7, using methyl 1-(3,3-difluoropropyl)-1H-1,2,4-triazole-3-carboxylate (Intermediate 57) in place of methyl 2-(3,3,3-trifluoropropyl)-2H-1,2,3-triazole-4-carboxylate, to provide the title compound as a white solid.

### Intermediate 59

### Methyl 1-(3,3-difluoropropyl)-1H-1,2,4-triazole-5-carboxylate

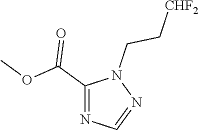

The title compound was prepared as described for the synthesis of Intermediate 57. Methyl 1-(3,3-difluoropropyl)-1H-1,2,4-triazole-5-carboxylate was the first eluting isomer, isolated as a light-yellow oil.

### Intermediate 60

### 1-((2-(Trimethylsilyl)ethoxy)methyl)-1H-benzo[d]imidazole-5-carbonitrile and 1-((2-(trimethylsilyl)ethoxy)methyl)-1H-benzo[d]imidazole-6-carbonitrile

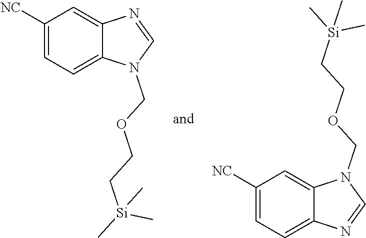 [SMILES: C[Si](C)(C)CCOCn1cnc2cc(C#N)ccc21 ; C[Si](C)(C)CCOCn1cnc2ccc(C#N)cc21]

To a mixture of 1H-benzo[d]imidazole-6-carbonitrile (30 g, 0.16 mol) in THF (500 mL) at 0° C. was added NaH (26 g, 0.65 mol, 60% in mineral oil) and the resulting mixture was warmed to rt over 1 h. Then, the mixture was cooled to 0° C. and SEMCl (32 g, 0.19 mol) was added dropwise. The reaction was stirred for 16 h while gradually warming to rt, and then poured into saturated aqueous ammonium chloride (600 mL) and extracted with EtOAc (3×600 mL). The organic layers were combined, washed with brine, dried over anhydrous Na₂SO₄, filtered, and concentrated to dryness. The crude material was purified by silica gel chromatography (0-60% EtOAc/petroleum ether) to provide a mixture of the title compounds as a red oil.

### Intermediate 61

### (R)—N—((S)-(5-Cyano-1-((2-(trimethylsilyl)ethoxy)methyl)-1H-benzo[d]imidazol-2-yl)(4,4-difluorocyclohexyl)methyl)-2-methylpropane-2-sulfinamide and (R)—N—((S)-(6-cyano-1-((2-(trimethylsilyl)ethoxy)methyl)-1H-benzo[d]imidazol-2-yl)(4,4-difluorocyclohexyl)methyl)-2-methylpropane-2-sulfinamide

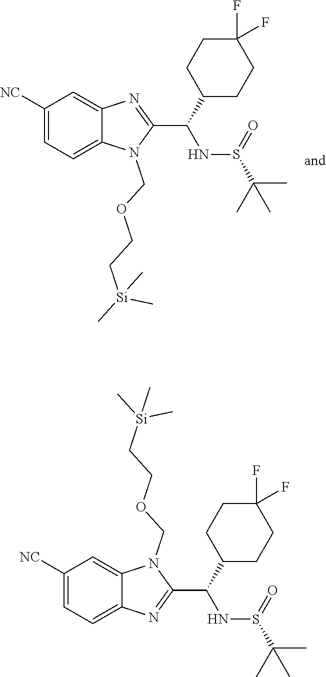 [SMILES: CC(C)(C)[S@@](=O)N[C@H](c1nc2cc(C#N)ccc2n1COCC[Si](C)(C)C)C1CCC(F)(F)CC1 ; CC(C)(C)[S@@](=O)N[C@H](c1nc2ccc(C#N)cc2n1COCC[Si](C)(C)C)C1CCC(F)(F)CC1]

To a solution of 1-((2-(trimethylsilyl)ethoxy)methyl)-1H-benzo[d]imidazole-5-carbonitrile and 1-((2-(trimethylsilyl)ethoxy)methyl)-1H-benzo[d]imidazole-6-carbonitrile (22.5 g, 82.3 mmol, Intermediate 60) in THF (300 mL) at −78° C. was added n-BuLi (38 mL, 95 mmol, 2.5 M in hexanes) and the resulting mixture was stirred at −78° C. for 30 min. Then, a solution of (R,Z)—N-((4,4-difluorocyclohexyl)methylene)-2-methylpropane-2-sulfinamide (23.8 g, 94.7 mmol, Intermediate 234) in THF (50 mL) was added via cannula and the mixture was stirred at −78° C. for 30 min. Then, the reaction was quenched by the addition of saturated aqueous NH₄Cl (500 mL) and extracted with EtOAc (3×500 mL). The organic layers were combined, washed with brine, dried over anhydrous Na₂SO₄, filtered, and concentrated to dryness to provide a mixture of the title compounds as a red oil.

### Intermediate 62

### (R)—N—((S)-(5-Cyano-1H-benzo[d]imidazol-2-yl)(4,4-difluorocyclohexyl)methyl)-2-methylpropane-2-sulfinamide

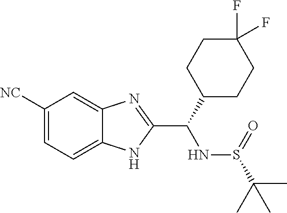 [SMILES: CC(C)(C)[S@@](=O)N[C@H](c1nc2cc(C#N)ccc2[nH]1)C1CCC(F)(F)CC1]

A mixture of (R)—N—((S)-(5-cyano-1-((2-(trimethylsilyl)ethoxy)methyl)-1H-benzo[d]imidazol-2-yl)(4,4-difluorocyclohexyl)methyl)-2-methylpropane-2-sulfinamide and (R)—N—((S)-(6-cyano-1-((2-(trimethylsilyl)ethoxy)methyl)-1H-benzo[d]imidazol-2-yl)(4,4-difluorocyclohexyl)methyl)-2-methylpropane-2-sulfinamide (47 g, 89.6 mmol, Intermediate 61) and TBAF (226 mL, 226 mmol, 1 M in THF) was heated to 90° C. for 16 h and then filtered through a pad of silica gel, washing the pad with 1:1 acetone/petroleum ether (250 mL). The filtrate was concentrated to dryness to provide the crude title compound as a red oil. The material was triturated with petroleum ether (150 mL) and EtOAc (15 mL) at 90° C. and then the mixture was filtered to provide the title compound as a white solid. The mother liquor was purified by preparative HPLC (Xbridge BEH 10 μm C18, 250×50 mm, 20-52% acetonitrile/water (with 0.04% NH₄OH and 10 mM NH₄HCO₃)). The product containing fractions were diluted with water, frozen, and lyophilized to afford the title compound as a white solid.

### Intermediate 63

### (S)-2-(Amino(4,4-difluorocyclohexyl)methyl)-1H-benzo[d]imidazole-5-carbonitrile hydrochloride

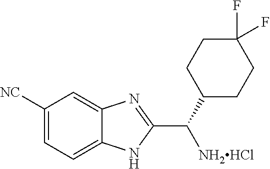

A solution of HCl in EtOAc (80 mL, 320 mmol, 4 M) was added to a solution of (R)—N—((S)-(5-cyano-1H-benzo[d]imidazol-2-yl)(4,4-difluorocyclohexyl)methyl)-2-methylpropane-2-sulfinamide (11 g, 27.9 mmol, Intermediate 62) in EtOAc (30 mL) at 0° C. and the resulting mixture was stirred at rt for 1.5 h. After that time, the mixture was concentrated to dryness to provide the title compound as a white solid.

### Intermediate 64

### (S)—N-((5-Cyano-1H-benzo[d]imidazol-2-yl)(4,4-difluorocyclohexyl)methyl)-1-methyl-1H-pyrazole-5-carboxamide

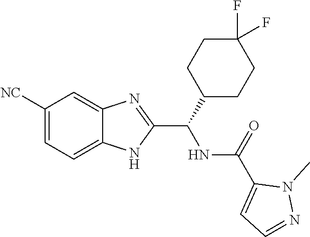 [SMILES: Cn1nccc1C(=O)N[C@H](c1nc2cc(C#N)ccc2[nH]1)C1CCC(F)(F)CC1]

A solution of 1-methyl-1H-pyrazole-5-carboxylic acid (2.8 g, 22.2 mmol) and HATU (9 g, 23.7 mmol) in DCM (200 mL) was stirred at 0° C. for 20 min. Then, (S)-2-(amino(4,4-difluorocyclohexyl)methyl)-1H-benzo[d]imidazole-5-carbonitrile hydrochloride (7 g, 21.4 mmol, Intermediate 63) and DIPEA (15 mL, 86.1 mmol) were added and the resulting mixture was stirred while warming to rt over 3 h. The mixture was then poured into water (250 mL) and extracted with EtOAc (3×250 mL). The organic layers were combined, washed with brine, dried over anhydrous Na₂SO₄, filtered, and concentrated to dryness. The residue was triturated with EtOAc (250 mL)/petroleum ether (250 mL) and filtered followed by purification via silica gel chromatography (50-100% EtOAc/petroleum ether) to provide the title compound as a white solid.

### Intermediate 65

### N-((1S)-(5-(Amino(cyclobutyl)methyl)-1H-benzo[d]imidazol-2-yl)(4,4-difluorocyclohexyl)methyl)-1-methyl-1H-pyrazole-5-carboxamide

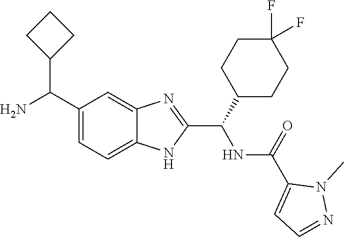 [SMILES: Cn1nccc1C(=O)N[C@H](c1nc2cc(C(N)C3CCC3)ccc2[nH]1)C1CCC(F)(F)CC1]

Cyclobutylmagnesium bromide (6.3 mL, 12.6 mmol, 2 M in THF) was added to a solution of (S)—N-((5-cyano-1H-benzo[d]imidazol-2-yl)(4,4-difluorocyclohexyl)methyl)-1-methyl-1H-pyrazole-5-carboxamide (500 mg, 1.25 mmol, Intermediate 64) and CuI (145 mg, 0.76 mmol) in THF (6 mL) under Ar. The resulting mixture was stirred at 100° C. for 20 min in the microwave. Then, the mixture was added to a solution of NaBH₄ (712 mg, 18.8 mmol) in MeOH (12.5 mL) and the resulting mixture was stirred at rt for 16 h. The reaction was quenched with water (20 mL) and filtered through a pad of Celite®, rinsing the pad with MeOH (30 mL). The filtrate was concentrated to dryness and then partitioned between water (15 mL) and EtOAc (20 mL). The aqueous layer was further extracted with EtOAc (2×20 mL), then the organic layers were combined, dried over anhydrous Na₂SO₄, filtered, and concentrated to dryness to provide the title compound as a light green solid.

### Intermediate 66

### N-((1S)-(5-(1-Amino-2-methylpropyl)-1H-benzo[d]imidazol-2-yl)(4,4-difluorocyclohexyl)methyl)-1-methyl-1H-pyrazole-5-carboxamide

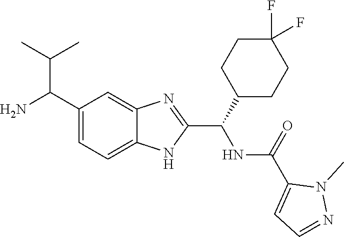 [SMILES: CC(C)C(N)c1ccc2[nH]c([C@@H](NC(=O)c3ccnn3C)C3CCC(F)(F)CC3)nc2c1]

The title compound was prepared as described for the synthesis of Intermediate 65, using isopropylmagnesium chloride lithium chloride in place of cyclobutylmagnesium bromide to afford the title compound, a mixture of diastereomers, as a light green solid.

### Intermediate 67

### N-((1S)-(5-(1-Amino-2-cyclobutylethyl)-1H-benzo[d]imidazol-2-yl)(4,4-difluorocyclohexyl)methyl)-1-methyl-1H-pyrazole-5-carboxamide

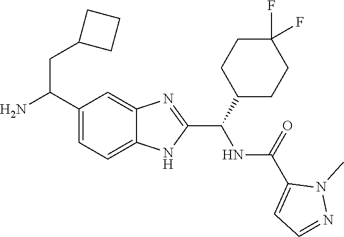 [SMILES: Cn1nccc1C(=O)N[C@H](c1nc2cc(C(N)CC3CCC3)ccc2[nH]1)C1CCC(F)(F)CC1]

The title compound was prepared as described for the synthesis of Intermediate 65, using (cyclobutylmethyl)magnesium bromide in place of cyclobutylmagnesium bromide and was purified by silica gel chromatography (30-100% EtOAc/petroleum ether) to provide the title compound, a mixture of diastereomers, as a white solid.

### Intermediate 68

### N-((1S)-(5-(1-Amino-3-methylbutyl)-1H-benzo[d]imidazol-2-yl)(4,4-difluorocyclohexyl)methyl)-1-methyl-1H-pyrazole-5-carboxamide

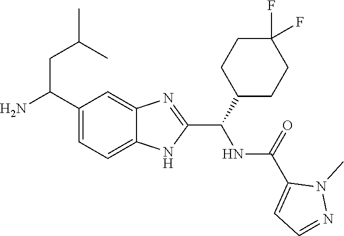 [SMILES: CC(C)CC(N)c1ccc2[nH]c([C@@H](NC(=O)c3ccnn3C)C3CCC(F)(F)CC3)nc2c1]

Isobutylmagnesium bromide (4 mL, 8 mmol, 2 M in Et₂O) was added to a solution of (S)—N-((5-cyano-1H-benzo[d]imidazol-2-yl)(4,4-difluorocyclohexyl)methyl)-1-methyl-1H-pyrazole-5-carboxamide (400 mg, 1 mmol, Intermediate 64) and CuCl (60 mg, 0.61 mmol) in THF (5 mL) under Ar and the resulting mixture was stirred at 100° C. for 20 min in the microwave. Then, the mixture was added to a solution of NaBH₄ (570 mg, 15.1 mmol) in MeOH (5 mL) and the resulting mixture was stirred at rt for 6 h. After that time, additional NaBH₄ (250 mg, 6.61 mmol) was added and the mixture stirred at rt for 19 h. The mixture was concentrated to dryness and then partitioned between water (30 mL) and EtOAc (40 mL). The aqueous layer was further extracted with EtOAc (2×40 mL), then the organic layers were combined, filtered through a pad of Celite®, rinsing with EtOAc (100 mL) and concentrated to dryness to provide the crude title compound (1.55 g) as a light green solid.

### Intermediate 69

### N-((1S)-(5-(1-Aminobutyl)-1H-benzo[d]imidazol-2-yl)(4,4-difluorocyclohexyl)methyl)-1-methyl-1H-pyrazole-5-carboxamide hydrochloride

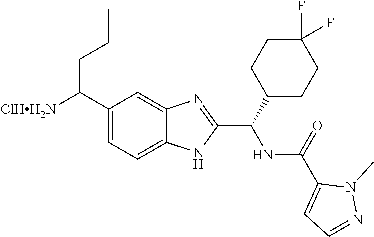

Propylmagnesium bromide (2.5 mL, 5 mmol, 2 M in THF) was added to a solution of (S)—N-((5-cyano-1H-benzo[d]imidazol-2-yl)(4,4-difluorocyclohexyl)methyl)-1-methyl-1H-pyrazole-5-carboxamide (400 mg, 1 mmol, Intermediate 64) in THF (3 mL) and the resulting mixture was stirred at 50° C. overnight. Then, NaBH₄ (380 mg, 10 mmol) was added in portions and the mixture was stirred at 50° C. for 4 h. The reaction was quenched with saturated aqueous NH₄Cl (10 mL) and extracted with EtOAc (3×15 mL). The organic layers were combined and concentrated to dryness. The residue was dissolved in an HCl solution (10 mL, 4 M in EtOAc) and the resulting solid was filtered to provide the title compound as a yellow solid.

### Intermediate 70

### N-((1S)-(5-(1-Aminopropyl)-1H-benzo[d]imidazol-2-yl)(4,4-difluorocyclohexyl)methyl)-1-methyl-1H-pyrazole-5-carboxamide hydrochloride

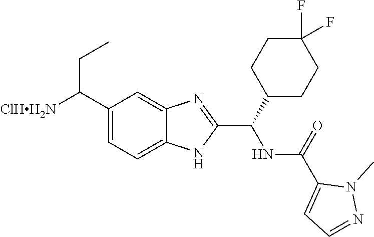

The title compound was prepared as described for the synthesis of Intermediate 69, using ethylmagnesium bromide in place of propylmagnesium bromide to provide the title compound, a mixture of diastereomers, as a light green solid.

### Intermediate 71

### N-((1S)-(5-(Amino(cyclopropyl)methyl)-1H-benzo[d]imidazol-2-yl)(4,4-difluorocyclohexyl)methyl)-1-methyl-1H-pyrazole-5-carboxamide

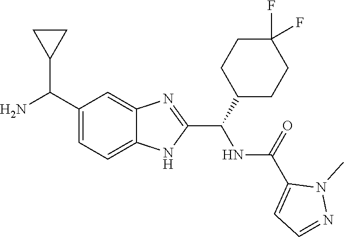 [SMILES: Cn1nccc1C(=O)N[C@H](c1nc2cc(C(N)C3CC3)ccc2[nH]1)C1CCC(F)(F)CC1]

Cyclopropylmagnesium bromide (8 mL, 4 mmol, 0.5 M in THF) was added to a solution of (S)—N-((5-cyano-1H-benzo[d]imidazol-2-yl)(4,4-difluorocyclohexyl)methyl)-1-methyl-1H-pyrazole-5-carboxamide (300 mg, 0.75 mmol, Intermediate 64) and CuCl (45 mg, 0.45 mmol) in THF (6 mL) under Ar and the resulting mixture was stirred at 100° C. for 20 min in the microwave. Then, the mixture was added to a solution of NaBH₄ (370 mg, 9.78 mmol) in MeOH (7.5 mL) and the resulting mixture was stirred at rt for 16 h. After that time, the mixture was quenched by the addition of MeOH (10 mL) and filtered through a pad of Celite®, rinsing with EtOAc (20 mL). The organic layer was dried over anhydrous Na₂SO₄, filtered, and concentrated to dryness to provide the title compound as a green solid.

### Intermediate 72

### 4-Carboxy-3-isopropyl-1,2,5-oxadiazole 2-oxide

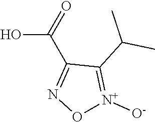

Jones reagent (2.3 mL, 4.6 mmol, 2 M in H₂SO₄) was added dropwise to a solution of 4-formyl-3-isopropyl-1,2,5-oxadiazole 2-oxide (450 mg, 2.9 mmol) in acetone (5.8 mL) at 0° C. The reaction was warmed to rt and stir for 2 h. After this time, the reaction solution was cooled to 0° C. and IPA (3 mL) was added and the mixture stirred for an additional 30 min. The solution was then concentrated under reduced pressure to remove organic solvents and was diluted with water and CH₂Cl₂. The biphasic solution was then extracted with 20% IPA in CH₂Cl₂ solution (4×15 mL). The combined organic layers were washed with brine, dried over anhydrous Na₂SO₄, filtered, and concentrated under reduced pressure to afford the crude title compound which was used without further purification.

### Intermediate 73

### 3-Isopropyl-4-(phenylcarbamoyl)-1,2,5-oxadiazole 2-oxide

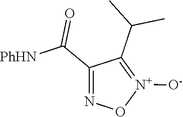

To a solution of 4-carboxy-3-isopropyl-1,2,5-oxadiazole 2-oxide (400 mg, 2.32 mmol, Intermediate 72) in DMF (11.6 mL) were added DIPEA (0.80 mL, 4.65 mmol) and HATU (1.17 g, 3.02 mmol) sequentially. The mixture was stirred for 3 min followed by the addition of aniline (0.30 mL, 3.25 mmol). The resulting mixture was stirred at rt for 2 h, then poured into a separatory funnel filled with water and was extracted with EtOAc (3×25 mL). The combined organic layers were washed with brine twice, dried over anhydrous MgSO₄, filtered, and concentrated under reduced pressure. The crude title compound was purified by silica gel chromatography (0-50% EtOAc/hexanes) to afford the title compound as a white solid.

### Intermediate 74

### 4-Isopropyl-N-phenyl-1,2,5-oxadiazole-3-carboxamide

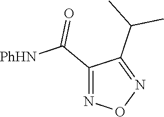

3-Isopropyl-4-(phenylcarbamoyl)-1,2,5-oxadiazole 2-oxide (630 mg, 2.55 mmol, Intermediate 73) was dissolved in toluene (12.7 mL) and was degassed with an inert N2 atmosphere. Trimethyl phosphite (6.0 mL, 51 mmol) was then added, dropwise, and the reaction was heated to 120° C. and stirred at that temperature for 12 h. The reaction was then cooled to rt and poured into a separatory funnel filled with 1 N aqueous HCl (50 mL). The biphasic mixture was extracted with EtOAc (3×50 mL) and the combined organic layers were washed with brine, dried over anhydrous MgSO₄, filtered, and concentrated under reduced pressure. The crude title compound was purified by silica gel chromatography (0-50% EtOAc/hexanes) to afford the pure title compound as an off-white solid.

### Intermediate 75

### tert-Butyl (4-isopropyl-1,2,5-oxadiazole-3-carbonyl)(phenyl)carbamate

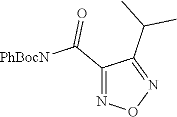

A flask was charged with 4-isopropyl-N-phenyl-1,2,5-oxadiazole-3-carboxamide (460 mg, 2.0 mmol, Intermediate 74) and DCM (10 mL). Di-tert-butyl dicarbonate (480 mg, 2.2 mmol) and DMAP (24 mg, 0.2 mmol) were sequentially added and the resultant solution was stirred at rt for 1 h. Silica gel was then added, and the resulting slurry was concentrated to dryness. Purification by silica gel chromatography (0-50% EtOAc/hexanes) afforded the title compound as a white solid.

### Intermediate 76

### 4-Isopropyl-1,2,5-oxadiazole-3-carboxylic acid

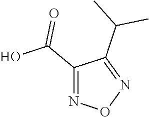

LiOH (10 mg, 0.43 mmol) was dissolved in deionized water (0.2 mL) and was added to a solution of tert-butyl (4-isopropyl-1,2,5-oxadiazole-3-carbonyl)(phenyl)carbamate (110 mg, 0.33 mmol, Intermediate 75) in THF (0.33 mL). The resulting reaction was stirred at rt for 1 h. The reaction was quenched with 1 N aqueous HCl (5 mL) and extracted with EtOAc (3×5 mL). The combined organic layers were then extracted with a saturated aqueous solution of NaHCO₃ (10 mL) and the organic layers were discarded. The basic aqueous layer was then slowly acidified to ˜pH 1 with 6 N aqueous HCl and extracted with EtOAc (3×10 mL). The combined organic layers were then dried with anhydrous Na₂SO₄, filtered, and concentrated under reduced pressure to afford the crude title compound and was used without further purification.

### Intermediate 77

### 3-Cyclopropyl-4-formyl-1,2,5-oxadiazole 2-oxide

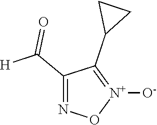 [SMILES: O=Cc1no[n+]([O-])c1C1CC1]

(E)-3-Cyclopropylacrylaldehyde (1.0 g, 10.4 mmol) was dissolved in glacial acetic acid (2 mL, 36 mmol) and cooled to 0° C. An aqueous solution of sodium nitrite (2.2 mL, 1.2 M) was then added dropwise via syringe pump (0.325 mL/min) and stirred at 0° C. for 1 h. The cooling bath was then removed, and the reaction stirred at rt overnight. The reaction was diluted with water (15 mL) and extracted with EtOAc (4×20 mL). The combined organic layers were washed with saturated aqueous NaHCO₃ and brine, dried over anhydrous MgSO₄, filtered, and concentrated under reduced pressure. The crude title compound was purified by silica gel chromatography (0-60% EtOAc/hexanes) to afford the title compound as a pale-yellow oil.

### Intermediate 78

### 4-Cyclopropyl-1,2,5-oxadiazole-3-carboxylic acid

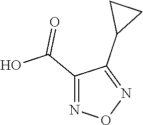 [SMILES: O=C(O)c1nonc1C1CC1]

The title compound was prepared as described for the synthesis of Intermediate 76, using 3-cyclopropyl-4-formyl-1,2,5-oxadiazole 2-oxide (Intermediate 77) in place of 4-formyl-3-isopropyl-1,2,5-oxadiazole 2-oxide.

### Intermediate 79

### 4-Methyl-1,2,5-oxadiazole-3-carbonyl chloride

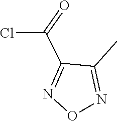

A flame-dried, round bottom flask was charged with 4-methyl-1,2,5-oxadiazole-3-carboxylic acid (6.40 g, 50 mmol), DCM (100 mL), and oxalyl chloride (8.63 mL, 100 mmol). The solution was cooled to 0° C. and to the solution was added DMF (0.39 mL, 5 mmol). The mixture was stirred for 4 h as it warmed to rt. Then, the mixture was concentrated into a yellow oil and dissolved in DCM to result in a 2 M solution of the title compound that was used in subsequent reactions without further purification.

### Intermediate 80

### 2,5-Dioxopyrrolidin-1-yl 4-methyl-1,2,5-oxadiazole-3-carboxylate

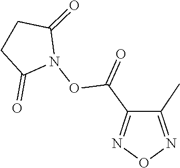 [SMILES: Cc1nonc1C(=O)ON1C(=O)CCC1=O]

A flame-dried round bottom flask was charged with N-hydroxysuccinimide (2.13 g, 18.0 mmol), DCM (30 mL), and DIPEA (3.10 mL, 18.0 mmol). The reaction was cooled to 0° C. and 4-methyl-1,2,5-oxadiazole-3-carbonyl chloride (6.0 mL, 12.0 mmol, Intermediate 79) was added dropwise. The reaction was stirred at rt overnight. Without adding additional solvent, the reaction mixture was washed with water and brine, dried over anhydrous MgSO₄, filtered, and concentrated. The crude material was purified by silica gel chromatography (0-100% EtOAc (with 10% MeOH)/hexanes) to afford the title compound as a clear oil.

### Intermediate 81

### Ethyl 2-(3,3-dimethylbutyl)-2H-1,2,3-triazole-4-carboxylate

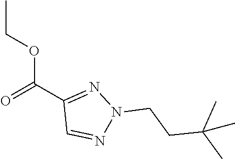 [SMILES: CCOC(=O)c1cnn(CCC(C)(C)C)n1]

The title compound was prepared as described for the synthesis of Intermediate 5, using ethyl 1H-1,2,3-triazole-4-carboxylate in place of methyl 1H-1,2,3-triazole-4-carboxylate and 1-bromo-3,3-dimethylbutane in place of 3-bromo-1,1,1-trifluoropropane, to provide the title compound (first eluting isomer) as a clear colorless oil.

### Intermediate 82

### Ethyl 1-(3,3-dimethylbutyl)-1H-1,2,3-triazole-5-carboxylate

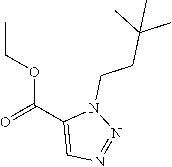 [SMILES: CCOC(=O)c1cnnn1CCC(C)(C)C]

The title compound was prepared as described for the synthesis of Intermediate 81. Ethyl 1-(3,3-dimethylbutyl)-1H-1,2,3-triazole-5-carboxylate was the second eluting isomer, isolated as a clear colorless oil.

### Intermediate 83

### 2-(3,3-Dimethylbutyl)-2H-1,2,3-triazole-4-carboxylic acid

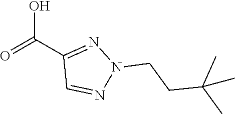

The title compound was prepared as described for the synthesis of Intermediate 7, using ethyl 2-(3,3-dimethylbutyl)-2H-1,2,3-triazole-4-carboxylate (Intermediate 81) in place of methyl 2-(3,3,3-trifluoropropyl)-2H-1,2,3-triazole-4-carboxylate, to provide the title compound as a white solid.

### Intermediate 84

### 1-(3,3-Dimethylbutyl)-1H-1,2,3-triazole-5-carboxylic acid

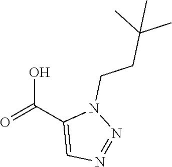

The title compound was prepared as described for the synthesis of Intermediate 7, using ethyl 1-(3,3-dimethylbutyl)-1H-1,2,3-triazole-5-carboxylate (Intermediate 82) in place of methyl 2-(3,3,3-trifluoropropyl)-2H-1,2,3-triazole-4-carboxylate, to provide the title compound as a white solid.

### Intermediate 85

### Ethyl 2-(3,3,3-trifluoro-2-methylpropyl)-2H-1,2,3-triazole-4-carboxylate

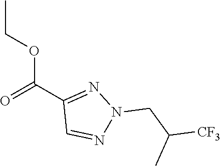 [SMILES: CCOC(=O)c1cnn(CC(C)C(F)(F)F)n1]

The title compound was prepared as described for the synthesis of Intermediate 15, using 3,3,3-trifluoro-2-methylpropan-1-ol in place of 2-cyclopropylethanol. Ethyl 2-(3,3,3-trifluoro-2-methylpropyl)-2H-1,2,3-triazole-4-carboxylate was the first eluting isomer, isolated as a clear colorless oil.

### Intermediate 86

### Ethyl 1-(3,3,3-trifluoro-2-methylpropyl)-1H-1,2,3-triazole-5-carboxylate

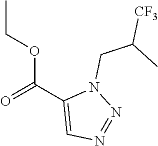 [SMILES: CCOC(=O)c1cnnn1CC(C)C(F)(F)F]

The title compound was prepared as described for the synthesis of Intermediate 85. Ethyl 1-(3,3,3-trifluoro-2-methylpropyl)-1H-1,2,3-triazole-5-carboxylate was the second eluting isomer, isolated as a light-yellow oil.

### Intermediate 87

### 2-(3,3,3-Trifluoro-2-methylpropyl)-2H-1,2,3-triazole-4-carboxylic acid

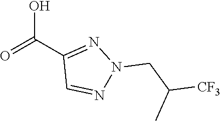

The title compound was prepared as described for the synthesis of Intermediate 7, using ethyl 2-(3,3,3-trifluoro-2-methylpropyl)-2H-1,2,3-triazole-4-carboxylate (Intermediate 85) in place of methyl 2-(3,3,3-trifluoropropyl)-2H-1,2,3-triazole-4-carboxylate, to provide the title compound as a white solid.

### Intermediate 88

### 1-(3,3,3-Trifluoro-2-methylpropyl)-1H-1,2,3-triazole-5-carboxylic acid

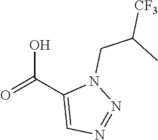

The title compound was prepared as described for the synthesis of Intermediate 7, using ethyl 1-(3,3,3-trifluoro-2-methylpropyl)-1H-1,2,3-triazole-5-carboxylate (Intermediate 86) in place of methyl 2-(3,3,3-trifluoropropyl)-2H-1,2,3-triazole-4-carboxylate, to provide the title compound as a white solid.

### Intermediate 89

### Ethyl 2-(cyclobutylmethyl)-2H-1,2,3-triazole-4-carboxylate

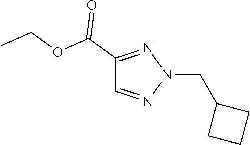

To a mixture of ethyl 1H-1,2,3-triazole-4-carboxylate (3.0 g, 21.3 mmol), K₂CO₃ (2.9 g, 21.3 mmol) and DMF (27.2 mL) was added (bromomethyl)cyclobutane (2.5 mL, 21.3 mmol) and the resulting mixture was stirred at rt for 20 h. After that time, the mixture was partitioned between EtOAc (30 mL) and water (30 mL). The layers were separated, and the aqueous layer further extracted with EtOAc (2×30 mL). The organic layers were combined, washed with water (30 mL) and then brine (30 mL), dried over anhydrous Na₂SO₄, filtered, and concentrated to dryness. The mixture of regioisomers was purified by silica gel chromatography (0-75% EtOAc/hexanes) to provide the title compound as the first eluting fraction.

### Intermediate 90

### 2-(Cyclobutylmethyl)-2H-1,2,3-triazole-4-carboxylic acid

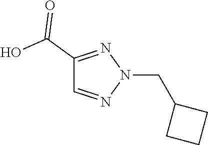 [SMILES: O=C(O)c1cnn(CC2CCC2)n1]

To a mixture of ethyl 2-(cyclobutylmethyl)-2H-1,2,3-triazole-4-carboxylate (1.74 g, 8.32 mmol, Intermediate 89) in THF (25 mL) was added 2 M aqueous NaOH (25 mL, 50 mmol) and the mixture was stirred at rt for 18 h. After that time, the mixture was concentrated to remove the THF and then washed with EtOAc. The aqueous layer was then acidified to pH 1-2 by the addition of 1 N aqueous HCl and the aqueous layer was extracted with EtOAc (3×30 mL) and the combined organic layers were washed with brine (30 mL), dried over anhydrous Na₂SO₄, filtered, and concentrated to dryness to provide the title compound as a white solid.

### Intermediate 91

### Methyl 2-((3,3-difluorocyclobutyl)methyl)-2H-1,2,3-triazole-4-carboxylate

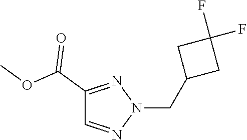 [SMILES: COC(=O)c1cnn(CC2CC(F)(F)C2)n1]

The title compound was prepared as described for the synthesis of Intermediate 89, using 3-(bromomethyl)-1,1-difluorocyclobutane in place of (bromomethyl)cyclobutane and methyl 1H-1,2,3-triazole-4-carboxylate in place of ethyl 1H-1,2,3-triazole-4-carboxylate. Methyl 2-((3,3-difluorocyclobutyl)methyl)-2H-1,2,3-triazole-4-carboxylate was the first eluting isomer, isolated as a clear colorless oil.

### Intermediate 92

### Methyl 1-((3,3-difluorocyclobutyl)methyl)-1H-1,2,3-triazole-5-carboxylate

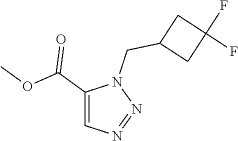 [SMILES: COC(=O)c1cnnn1CC1CC(F)(F)C1]

The title compound was prepared as described for the synthesis of Intermediate 91. Methyl 1-((3,3-difluorocyclobutyl)methyl)-1H-1,2,3-triazole-5-carboxylate was the second eluting isomer and was isolated as a clear colorless oil.

### Intermediate 93

### Methyl 1-((3,3-difluorocyclobutyl)methyl)-1H-1,2,3-triazole-4-carboxylate

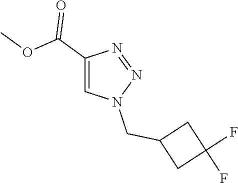 [SMILES: COC(=O)c1cn(CC2CC(F)(F)C2)nn1]

The title compound was prepared as described for the synthesis of Intermediate 91. Methyl 1-((3,3-difluorocyclobutyl)methyl)-1H-1,2,3-triazole-4-carboxylate was the third eluting isomer, isolated as a clear colorless oil.

### Intermediate 94

### 2-((3,3-Difluorocyclobutyl)methyl)-2H-1,2,3-triazole-4-carboxylic acid

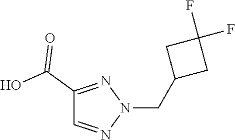 [SMILES: O=C(O)c1cnn(CC2CC(F)(F)C2)n1]

The title compound was prepared as described for the synthesis of Intermediate 90, using methyl 2-((3,3-difluorocyclobutyl)methyl)-2H-1,2,3-triazole-4-carboxylate (Intermediate 91) in place of ethyl 2-(cyclobutylmethyl)-2H-1,2,3-triazole-4-carboxylate to provide the title compound as a white solid.

### Intermediate 95

### 1-((3,3-Difluorocyclobutyl)methyl)-1H-1,2,3-triazole-5-carboxylic acid

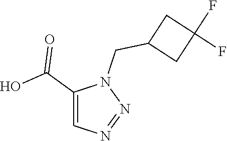 [SMILES: O=C(O)c1cnnn1CC1CC(F)(F)C1]

The title compound was prepared as described for the synthesis of Intermediate 90, using methyl 1-((3,3-difluorocyclobutyl)methyl)-1H-1,2,3-triazole-5-carboxylate (Intermediate 92) in place of ethyl 2-(cyclobutylmethyl)-2H-1,2,3-triazole-4-carboxylate to provide the title compound as a white solid.

### Intermediate 96

### 1-((3,3-Difluorocyclobutyl)methyl)-1H-1,2,3-triazole-4-carboxylic acid

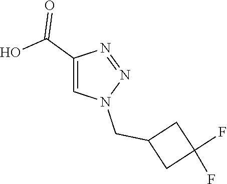 [SMILES: O=C(O)c1cn(CC2CC(F)(F)C2)nn1]

The title compound was prepared as described for the synthesis of Intermediate 90, using methyl 1-((3,3-difluorocyclobutyl)methyl)-1H-1,2,3-triazole-4-carboxylate (Intermediate 93) in place of ethyl 2-(cyclobutylmethyl)-2H-1,2,3-triazole-4-carboxylate to provide the title compound as a white solid.

### Intermediate 97

### Ethyl 2-(3,3-difluoropropyl)-2H-1,2,3-triazole-4-carboxylate

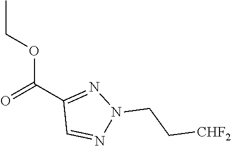

To a vial was added ethyl 1H-1,2,3-triazole-4-carboxylate (1.0 g, 6.93 mmol), 3,3-difluoropropan-1-ol (1.0 g, 10.4 mmol), PPh₃ (2.0 g, 7.6 mmol) and THF (23 mL). Then, the mixture was cooled to 0° C. and DIAD (1.5 mL, 7.6 mmol) was added and the resulting mixture stirred at rt for 2.5 h. The reaction mixture was concentrated to dryness and the residue purified by silica gel chromatography (0-100% EtOAc/hexanes) to provide the title compound (first eluting isomer) as a clear colorless oil.

### Intermediate 98

### Ethyl 1-(3,3-difluoropropyl)-1H-1,2,3-triazole-5-carboxylate

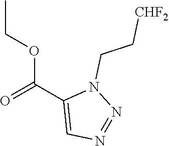

The title compound was prepared as described for the synthesis of Intermediate 97. Ethyl 1-(3,3-difluoropropyl)-1H-1,2,3-triazole-5-carboxylate was the second eluting isomer, isolated as a clear light-yellow oil.

### Intermediate 99

### 2-(3,3-Difluoropropyl)-2H-1,2,3-triazole-4-carboxylic acid

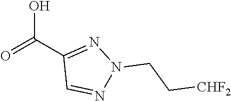 [SMILES: O=C(O)c1cnn(CCC(F)F)n1]

The title compound was prepared as described for the synthesis of Intermediate 90, using ethyl 2-(3,3-difluoropropyl)-2H-1,2,3-triazole-4-carboxylate (Intermediate 97) in place of ethyl 2-(cyclobutylmethyl)-2H-1,2,3-triazole-4-carboxylate, and stirring at rt for 2 h instead of 18 h, to provide the title compound as a white solid.

### Intermediate 100

### 1-(3,3-Difluoropropyl)-1H-1,2,3-triazole-5-carboxylic acid

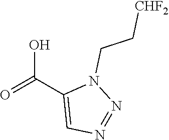 [SMILES: O=C(O)c1cnnn1CCC(F)F]

The title compound was prepared as described for the synthesis of Intermediate 90, using ethyl 1-(3,3-difluoropropyl)-1H-1,2,3-triazole-5-carboxylate (Intermediate 98) in place of ethyl 2-(cyclobutylmethyl)-2H-1,2,3-triazole-4-carboxylate to provide the title compound as a white solid.

### Intermediate 101

### (3-Cyanobicyclo[1.1.1]pentan-1-yl)methyl 4-bromobenzenesulfonate

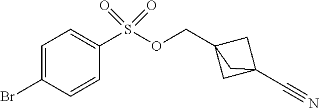 [SMILES: N#CC12CC(COS(=O)(=O)c3ccc(Br)cc3)(C1)C2]

3-(Hydroxymethyl)bicyclo[1.1.1]pentane-1-carbonitrile (959 mg, 7.79 mmol), 4-bromobenzenesulfonyl chloride (2.23 g, 8.74 mmol), DCM (16 mL), and Et₃N (1.7 mL, 12 mmol) were added to a 40 mL vial, and the resultant mixture stirred at rt for 22 h. The mixture was then diluted with EtOAc, washed with 1 N aqueous HCl and brine, dried over anhydrous MgSO₄, filtered, and concentrated to dryness to give the crude product. The crude product was purified by silica gel chromatography (0-30% EtOAc/hexanes) to provide the title compound as a white solid.

### Intermediate 102

### Ethyl 2-((3-cyanobicyclo[1.1.1]pentan-1-yl)methyl)-2H-1,2,3-triazole-4-carboxylate

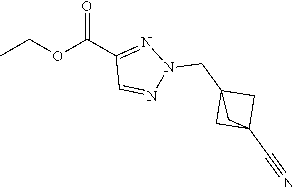 [SMILES: CCOC(=O)c1cnn(CC23CC(C#N)(C2)C3)n1]

NaH (154 mg, 3.84 mmol, 60% dispersion in mineral oil) and DMF (20 mL) were added to a nitrogen-purged 200 mL round-bottomed flask. The mixture was then treated with a solution consisting of (3-cyanobicyclo[1.1.1]pentan-1-yl)methyl 4-bromobenzenesulfonate (1.00 g, 2.94 mmol, Intermediate 101), ethyl 1H-1,2,3-triazole-5-carboxylate (460 mg, 3.26 mmol), and DMF (10 mL) drop-wise over 7 min. The flask that originally held the (3-cyanobicyclo[1.1.1]pentan-1-yl)methyl 4-bromobenzenesulfonate and triazole was rinsed with DMF (5 mL), and the DMF transferred to the reaction vessel via syringe. Stirring was continued at rt for 5 min and then heated at 80° C. for 8 h before cooling to rt, and slowly treated with water (dropwise) until effervescence ceased. The mixture was then diluted with EtOAc, washed with water (×3), dried over anhydrous MgSO₄, filtered, and concentrated to dryness to give a yellow-brown oil. The oil was purified by silica gel chromatography (0-50% EtOAc/hexanes) to provide the title compound (first eluting isomer) as a colorless oil.

### Intermediate 103

### 2-((3-Cyanobicyclo[1.1.1]pentan-1-yl)methyl)-2H-1,2,3-triazole-4-carboxylic acid

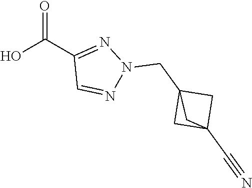 [SMILES: N#CC12CC(Cn3ncc(C(=O)O)n3)(C1)C2]

Ethyl 2-((3-cyanobicyclo[1.1.1]pentan-1-yl)methyl)-2H-1,2,3-triazole-4-carboxylate (255 mg, 1.04 mmol, Intermediate 102) and EtOH (2.4 mL) were added to a 20 mL vial and the mixture sonicated until a homogeneous solution was obtained. The mixture was then treated with 2 M KOH in EtOH (1.2 mL, 2.04 mmol) and heated at 60° C. for 24 h. The vial was cooled to rt, and the EtOH removed in vacuo. Water (2 mL) was added to the vial, and the mixture treated dropwise with 1 N aqueous HCl until a white precipitate formed. The solid obtained was the title compound and was isolated via vacuum filtration. The filtrate was then extracted with EtOAc (3×50 mL), and the combined extracts dried over anhydrous MgSO₄, filtered, and concentrated to dryness to afford an additional portion of the title compound as a white solid.

### Intermediate 104

### Ethyl 1-((3-cyanobicyclo[1.1.1]pentan-1-yl)methyl)-1H-1,2,3-triazole-4-carboxylate

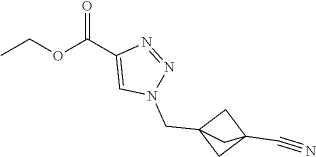 [SMILES: CCOC(=O)c1cn(CC23CC(C#N)(C2)C3)nn1]

The title compound was prepared as described for the synthesis of Intermediate 102. Ethyl 1-((3-cyanobicyclo[1.1.1]pentan-1-yl)methyl)-1H-1,2,3-triazole-4-carboxylate was the third eluting isomer, isolated as a pale yellow solid.

### Intermediate 105

### 1-((3-Cyanobicyclo[1.1.1]pentan-1-yl)methyl)-1H-1,2,3-triazole-4-carboxylic acid

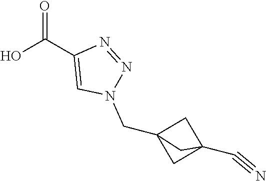 [SMILES: N#CC12CC(Cn3cc(C(=O)O)nn3)(C1)C2]

The title compound was prepared as described for the synthesis of Intermediate 103, using ethyl 1-((3-cyanobicyclo[1.1.1]pentan-1-yl)methyl)-1H-1,2,3-triazole-4-carboxylate (Intermediate 104) in place of ethyl 2-((3-cyanobicyclo[1.1.1]pentan-1-yl)methyl)-2H-1,2,3-triazole-4-carboxylate, to provide the title compound as a white solid.

### Intermediate 106

### (2,2-Difluorocyclobutyl)methyl 4-bromobenzenesulfonate

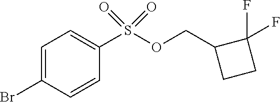 [SMILES: O=S(=O)(OCC1CCC1(F)F)c1ccc(Br)cc1]

The title compound was prepared as described for the synthesis of Intermediate 101, using (2,2-difluorocyclobutyl)methanol in place of 3-(hydroxymethyl)bicyclo[1.1.1]pentane-1-carbonitrile, to provide the title compound as a white solid.

### Intermediate 107

### Ethyl 1-((2,2-difluorocyclobutyl)methyl)-1H-1,2,3-triazole-5-carboxylate

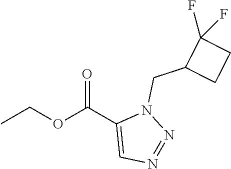 [SMILES: CCOC(=O)c1cnnn1CC1CCC1(F)F]

The title compound was prepared as described for the synthesis of Intermediate 102, using (2,2-difluorocyclobutyl)methyl 4-bromobenzenesulfonate (Intermediate 106) in place of (3-cyanobicyclo[1.1.1]pentan-1-yl)methyl 4-bromobenzenesulfonate. Ethyl 1-((2,2-difluorocyclobutyl)methyl)-1H-1,2,3-triazole-5-carboxylate was the second eluting isomer, isolated as a colorless oil.

### Intermediate 108

### 1-((2,2-Difluorocyclobutyl)methyl)-1H-1,2,3-triazole-5-carboxylic acid

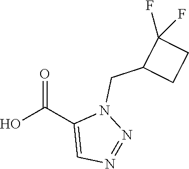 [SMILES: O=C(O)c1cnnn1CC1CCC1(F)F]

Ethyl 1-((2,2-difluorocyclobutyl)methyl)-1H-1,2,3-triazole-5-carboxylate (95 mg, 0.39 mmol, Intermediate 107), THF (1.2 mL), and 2 M aqueous NaOH (1.2 mL, 2.4 mmol) were added to a 20 mL vial, and the mixture was stirred at rt for 21 h before removing the THF in vacuo. The resulting aqueous solution was treated with 1 N aqueous HCl to pH ˜1 and a white solid (title compound) precipitated out of solution. The solid was isolated via vacuum filtration, and the filtrate extracted with EtOAc (×2). The combined organic extracts were dried over anhydrous MgSO₄, filtered, and concentrated to dryness to afford additional title compound as a white solid.

### Intermediate 109

### Ethyl 2-((2,2-difluorocyclobutyl)methyl)-2H-1,2,3-triazole-4-carboxylate

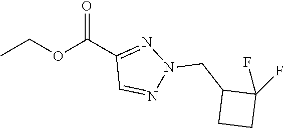 [SMILES: CCOC(=O)c1cnn(CC2CCC2(F)F)n1]

The title compound was prepared as described for the synthesis of Intermediate 107. Ethyl 2-((2,2-difluorocyclobutyl)methyl)-2H-1,2,3-triazole-4-carboxylate was the first eluting isomer, isolated as a colorless oil.

### Intermediate 110

### 2-((2,2-Difluorocyclobutyl)methyl)-2H-1,2,3-triazole-4-carboxylic acid

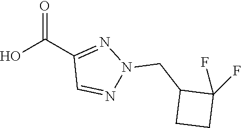 [SMILES: O=C(O)c1cnn(CC2CCC2(F)F)n1]

The title compound was prepared as described for the synthesis of Intermediate 108, using ethyl 2-((2,2-difluorocyclobutyl)methyl)-2H-1,2,3-triazole-4-carboxylate (Intermediate 109) in place of ethyl 1-((2,2-difluorocyclobutyl)methyl)-1H-1,2,3-triazole-5-carboxylate, to provide the title compound as a white solid.

### Intermediate 111

### Ethyl 1-((2,2-difluorocyclopropyl)methyl)-1H-1,2,3-triazole-5-carboxylate

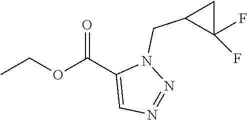 [SMILES: CCOC(=O)c1cnnn1CC1CC1(F)F]

Triphenylphosphine (3.03 g, 11.6 mmol) and THF (13.0 mL) were added to a nitrogen-purged, 100 mL, round-bottomed flask. The flask was cooled to 0° C. and charged with DIAD (2.2 mL, 11.3 mmol) dropwise over the course of 6 min, which gave rise to an off-white precipitate. The heterogeneous mixture was stirred for 10 min before adding a solution of 2,2-difluorocyclopropylmethanol (1.01 g, 9.32 mmol), ethyl 1H-1,2,3-triazole-4-carboxylate (1.33 g, 9.41 mmol) and THF (10 mL) dropwise over 9 min. The mixture was stirred for 14 h with gradual warming to rt. The reaction mixture was concentrated, dissolved in EtOAc, washed with 1 N aqueous NaOH and brine, dried over anhydrous MgSO₄, filtered, and concentrated to afford a viscous oil. The crude product was purified by silica gel chromatography (0-25% EtOAc/hexanes) to afford the title compound, the second eluting isomer, as a colorless oil.

### Intermediate 112

### 1-((2,2-Difluorocyclopropyl)methyl)-1H-1,2,3-triazole-5-carboxylic acid

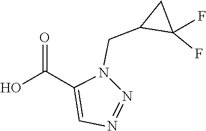 [SMILES: O=C(O)c1cnnn1CC1CC1(F)F]

The title compound was prepared as described for the synthesis of Intermediate 108, using ethyl 1-((2,2-difluorocyclopropyl)methyl)-1H-1,2,3-triazole-5-carboxylate (Intermediate 111) in place of ethyl 1-((2,2-difluorocyclobutyl)methyl)-1H-1,2,3-triazole-5-carboxylate, to provide the title compound as a white solid.

### Intermediate 113

### Ethyl 2-((2,2-difluorocyclopropyl)methyl)-2H-1,2,3-triazole-4-carboxylate

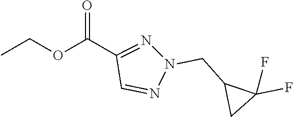 [SMILES: CCOC(=O)c1cnn(CC2CC2(F)F)n1]

The title compound was prepared as described for the synthesis of Intermediate 111. Ethyl 2-((2,2-difluorocyclopropyl)methyl)-2H-1,2,3-triazole-4-carboxylate was the first eluting isomer, isolated as a colorless oil.

### Intermediate 114

### 2-((2,2-Difluorocyclopropyl)methyl)-2H-1,2,3-triazole-4-carboxylic acid

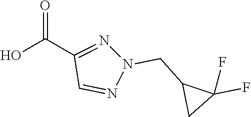 [SMILES: O=C(O)c1cnn(CC2CC2(F)F)n1]

The title compound was prepared as described for the synthesis of Intermediate 108, using ethyl 2-((2,2-difluorocyclopropyl)methyl)-2H-1,2,3-triazole-4-carboxylate (Intermediate 113) in place of ethyl 1-((2,2-difluorocyclobutyl)methyl)-1H-1,2,3-triazole-5-carboxylate, to provide the title compound as a white solid.

### Intermediate 115

### (2,2,3,3-Tetrafluorocyclobutyl)methyl 4-methylbenzenesulfonate

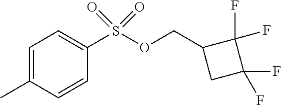 [SMILES: Cc1ccc(S(=O)(=O)OCC2CC(F)(F)C2(F)F)cc1]

2,2,3,3-Tetrafluorocyclobutylmethanol (0.92 g, 5.83 mmol), TsCl (1.37 g, 7.17 mmol), DCM (6.5 mL), and pyridine (0.60 mL, 7.4 mmol) were added to a 20 mL vial, and the resultant mixture stirred at rt for 22 h. The mixture was then diluted with EtOAc, washed with 1 N aqueous NaOH, water, and brine, dried over anhydrous MgSO₄, filtered, and concentrated to dryness. The crude product was purified by silica gel chromatography (0-10% EtOAc/hex) to afford the title compound as a white solid.

### Intermediate 116

### Ethyl 1-((2,2,3,3-tetrafluorocyclobutyl)methyl)-1H-1,2,3-triazole-5-carboxylate

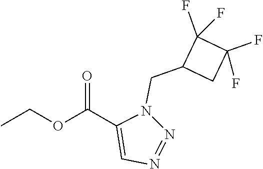 [SMILES: CCOC(=O)c1cnnn1CC1CC(F)(F)C1(F)F]

(2,2,3,3-Tetrafluorocyclobutyl)methyl 4-methylbenzenesulfonate (603 mg, 1.93 mmol, Intermediate 115), ethyl 1H-1,2,3-triazole-5-carboxylate (281 mg, 1.99 mmol), K₂CO₃ (546 mg, 3.95 mmol) and DMF (4 mL) were added to a 20 mL vial and the mixture stirred at rt for 15 h. The mixture was then diluted with EtOAc and washed three times with water followed by brine. The organic layer was dried over anhydrous MgSO₄, filtered, and concentrated to dryness. The crude product was purified by silica gel chromatography (0-100% EtOAc/hexanes) to provide the title compound, the second eluting isomer, as a white solid.

### Intermediate 117

### 1-((2,2,3,3-Tetrafluorocyclobutyl)methyl)-1H-1,2,3-triazole-5-carboxylic acid

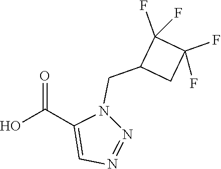 [SMILES: O=C(O)c1cnnn1CC1CC(F)(F)C1(F)F]

The title compound was prepared as described for the synthesis of Intermediate 108, using ethyl 1-((2,2,3,3-tetrafluorocyclobutyl)methyl)-1H-1,2,3-triazole-5-carboxylate (Intermediate 116) in place of ethyl 1-((2,2-difluorocyclobutyl)methyl)-1H-1,2,3-triazole-5-carboxylate, to provide the title compound as a white solid.

### Intermediate 118

### Ethyl 2-((2,2,3,3-tetrafluorocyclobutyl)methyl)-2H-1,2,3-triazole-4-carboxylate

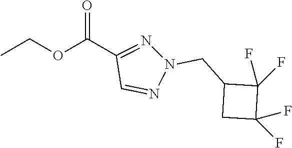 [SMILES: CCOC(=O)c1cnn(CC2CC(F)(F)C2(F)F)n1]

The title compound was prepared as described for the synthesis of Intermediate 116. Ethyl 2-((2,2,3,3-tetrafluorocyclobutyl)methyl)-2H-1,2,3-triazole-4-carboxylate was the first eluting isomer, isolated as a white solid.

### Intermediate 119

### 2-((2,2,3,3-Tetrafluorocyclobutyl)methyl)-2H-1,2,3-triazole-4-carboxylic acid

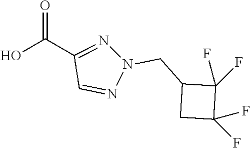 [SMILES: O=C(O)c1cnn(CC2CC(F)(F)C2(F)F)n1]

The title compound was prepared as described for the synthesis of Intermediate 108, using ethyl 2-((2,2,3,3-tetrafluorocyclobutyl)methyl)-2H-1,2,3-triazole-4-carboxylate (Intermediate 118) in place of ethyl 1-((2,2-difluorocyclobutyl)methyl)-1H-1,2,3-triazole-5-carboxylate, to provide the title compound as a white solid.

### Intermediate 120

### Ethyl 1-((2,2,3,3-tetrafluorocyclobutyl)methyl)-1H-1,2,3-triazole-4-carboxylate

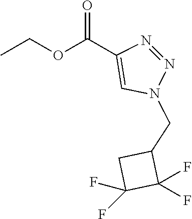 [SMILES: CCOC(=O)c1cn(CC2CC(F)(F)C2(F)F)nn1]

The title compound was prepared as described for the synthesis of Intermediate 116. Ethyl 1-((2,2,3,3-tetrafluorocyclobutyl)methyl)-1H-1,2,3-triazole-4-carboxylate was the third eluting isomer, isolated as a white solid.

### Intermediate 121

### 1-((2,2,3,3-Tetrafluorocyclobutyl)methyl)-1H-1,2,3-triazole-4-carboxylic acid

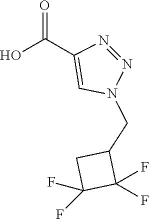 [SMILES: O=C(O)c1cn(CC2CC(F)(F)C2(F)F)nn1]

The title compound was prepared as described for the synthesis of Intermediate 108, using ethyl 1-((2,2,3,3-tetrafluorocyclobutyl)methyl)-1H-1,2,3-triazole-4-carboxylate (Intermediate 120) in place of ethyl 1-((2,2-difluorocyclobutyl)methyl)-1H-1,2,3-triazole-5-carboxylate, to provide the title compound as a white solid.

### Intermediate 122

### Ethyl 3-cyano-1-(cyclobutylmethyl)-1H-pyrazole-4-carboxylate

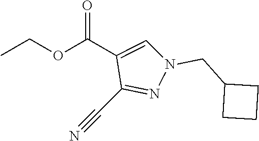 [SMILES: CCOC(=O)c1cn(CC2CCC2)nc1C#N]

The title compound was prepared as described for the synthesis of Intermediate 116, using (bromomethyl)cyclobutane in place of (2,2,3,3-tetrafluorocyclobutyl)methyl 4-methylbenzenesulfonate and ethyl 3-cyano-1H-pyrazole-4-carboxylate in place of ethyl 1H-1,2,3-triazole-5-carboxylate, to provide the title compound as a white solid.

### Intermediate 123

### Potassium 3-cyano-1-(cyclobutylmethyl)-1H-pyrazole-4-carboxylate and potassium 3-carbamoyl-1-(cyclobutylmethyl)-1H-pyrazole-4-carboxylate

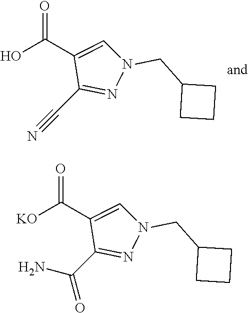

Ethyl 3-cyano-1-(cyclobutylmethyl)-1H-pyrazole-4-carboxylate (57 mg, 0.24 mmol, Intermediate 122) was added to a 25 mL round-bottomed flask, which was subsequently purged with nitrogen and cooled to 0° C. A freshly prepared solution of 0.1 M KOH in ethanol (5.7 mL) was added to the flask, and the resulting mixture stirred at 0° C. for 4 h before warming to rt, and stirring an additional 71 h. The mixture was then concentrated to dryness in vacuo and without heating to give a white solid. The solid was triturated with Et₂O (3×3 mL) and DCM (3 mL) and then dried under high-vacuum to afford a white solid consisting of a 1:1 mixture of the two title compounds.

### Intermediate 124

### Methyl 5-hydroxy-1-(3,3,3-trifluoropropyl)-1H-pyrazole-3-carboxylate

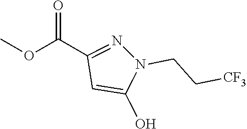 [SMILES: COC(=O)c1cc(O)n(CCC(F)(F)F)n1]

The title compound was prepared as described for the synthesis of Intermediate 116, using 3-bromo-1,1,1-trifluoropropane in place of (2,2,3,3-tetrafluorocyclobutyl)methyl 4-methylbenzenesulfonate and methyl 5-oxo-2,5-dihydro-1H-pyrazole-3-carboxylate in place of ethyl 1H-1,2,3-triazole-5-carboxylate, to provide the title compound as a white solid.

### Intermediate 125

### Potassium 5-hydroxy-1-(3,3,3-trifluoropropyl)-1H-pyrazole-3-carboxylate

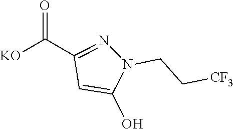

The title compound was prepared as described for the synthesis of Intermediate 123, using methyl 5-hydroxy-1-(3,3,3-trifluoropropyl)-1H-pyrazole-3-carboxylate (Intermediate 124) in place of ethyl 3-cyano-1-(cyclobutylmethyl)-1H-pyrazole-4-carboxylate, to provide the title compound as a white solid.

### Intermediate 126

### Ethyl 4-cyano-1-(cyclobutylmethyl)-1H-pyrazole-5-carboxylate

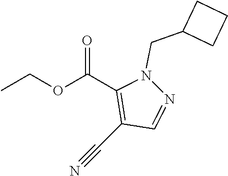 [SMILES: CCOC(=O)c1c(C#N)cnn1CC1CCC1]

The title compound was prepared as described for the synthesis of Intermediate 116, using (bromomethyl)cyclobutane in place of (2,2,3,3-tetrafluorocyclobutyl)methyl 4-methylbenzenesulfonate and ethyl 4-cyano-1H-pyrazole-5-carboxylate in place of ethyl 1H-1,2,3-triazole-5-carboxylate. Ethyl 4-cyano-1-(cyclobutylmethyl)-1H-pyrazole-5-carboxylate was the first eluting isomer, isolated as a white solid.

### Intermediate 127

### Potassium 4-cyano-1-(cyclobutylmethyl)-1H-pyrazole-5-carboxylate

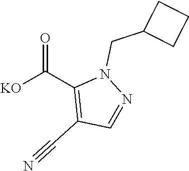

The title compound was prepared as described for the synthesis of Intermediate 123, using ethyl 4-cyano-1-(cyclobutylmethyl)-1H-pyrazole-5-carboxylate (Intermediate 126) in place of ethyl 3-cyano-1-(cyclobutylmethyl)-1H-pyrazole-4-carboxylate, to provide the title compound as a white solid.

### Intermediate 128

### Ethyl 4-cyano-1-(cyclobutylmethyl)-1H-pyrazole-3-carboxylate

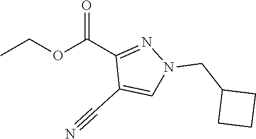 [SMILES: CCOC(=O)c1nn(CC2CCC2)cc1C#N]

The title compound was prepared as described for the synthesis of Intermediate 126. Ethyl 4-cyano-1-(cyclobutylmethyl)-1H-pyrazole-3-carboxylate was the second eluting isomer, isolated as a white solid.

### Intermediate 129

### 4-Cyano-1-(cyclobutylmethyl)-1H-pyrazole-3-carboxylic acid

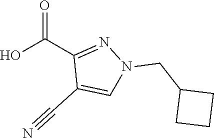 [SMILES: N#Cc1cn(CC2CCC2)nc1C(=O)O]

The title compound was prepared as described for the synthesis of Intermediate 103, using ethyl 4-cyano-1-(cyclobutylmethyl)-1H-pyrazole-3-carboxylate (Intermediate 128) in place of ethyl 2-((3-cyanobicyclo[1.1.1]pentan-1-yl)methyl)-2H-1,2,3-triazole-4-carboxylate, to provide the title compound as a white solid.

### Intermediate 130

### 1,1-Difluoropropan-2-yl 4-methylbenzenesulfonate

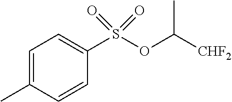 [SMILES: Cc1ccc(S(=O)(=O)OC(C)C(F)F)cc1]

The title compound was prepared as described for the synthesis of Intermediate 115, using 1,1-difluoropropan-2-ol in place of 2,2,3,3-tetrafluorocyclobutylmethanol, to provide the title compound as a colorless oil.

### Intermediate 131

### Ethyl 1-(1,1-difluoropropan-2-yl)-1H-1,2,3-triazole-4-carboxylate

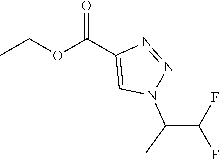

The title compound was prepared as described for the synthesis of Intermediate 116, using 1,1-difluoropropan-2-yl 4-methylbenzenesulfonate (Intermediate 130) in place of (2,2,3,3-tetrafluorocyclobutyl)methyl 4-methylbenzenesulfonate, to provide the title compound as a white solid.

### Intermediate 132

### 1-(1,1-Difluoropropan-2-yl)-1H-1,2,3-triazole-4-carboxylic acid

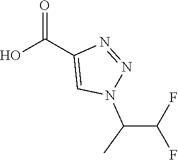

The title compound was prepared as described for the synthesis of Intermediate 108, using ethyl 1-(1,1-difluoropropan-2-yl)-1H-1,2,3-triazole-4-carboxylate (Intermediate 131) in place of ethyl 1-((2,2-difluorocyclobutyl)methyl)-1H-1,2,3-triazole-5-carboxylate, to provide the title compound as a white solid.

### Intermediate 133

### (R)—N-Methoxy-N-methyl-3-oxocyclohexane-1-carboxamide

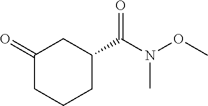

(1R)-3-Oxo-cyclohexanecarboxylic acid (9.17 g, 64.5 mmol), N,O-dimethylhydroxylamine hydrochloride (8.05 g, 82.6 mmol), HATU (37.4 g, 98.3 mmol), DCM (255 mL) and DIPEA (33 mL) were added to a 1 L round-bottomed flask, and the resultant mixture stirred for 18.5 h and then concentrated to dryness. The crude product was dissolved in a minimum amount of DCM, subjected to vacuum filtration and the filtrate concentrated to near dryness. The concentrated solution was purified by silica gel chromatography (0-100% EtOAc/hexanes) to provide the title compound as a pale-yellow oil.

### Intermediate 134

### (R)-3,3-Difluoro-N-methoxy-N-methylcyclohexane-1-carboxamide

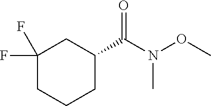

(R)—N-Methoxy-N-methyl-3-oxocyclohexane-1-carboxamide (9.58 g, 51.7 mmol, Intermediate 133), and DCM (520 mL) were added to a 1 L round-bottomed flask, which was subsequently purged with nitrogen and cooled to −78° C. before adding DAST (31.5 mL, 257 mmol), dropwise over 35 min. The mixture was gradually warmed to rt and stirring continued for 26.5 h. The reaction flask was then re-cooled to −78° C. and sparged with ozone until the mixture became a persistent blue-grey color. Dimethyl sulfide was then added dropwise until the grey color disappeared, and then more rapidly (˜3.0 mL). The mixture was stirred for ˜2 h while gradually warming to rt then diluted with DCM (˜200 mL) and washed with saturated aqueous NaHCO₃ followed by brine. The organic layer was then dried over anhydrous MgSO₄, filtered, and concentrated to dryness to give a brown oil. The crude product was purified by silica gel chromatography (0-100% DCM/hexanes) to provide the title compound as a pale-yellow oil.

### Intermediate 135

### (R)-3,3-Difluorocyclohexane-1-carbaldehyde

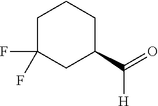

THF (175 mL) was added to a nitrogen-purged 500 mL round-bottomed flask containing (R)-3,3-difluoro-N-methoxy-N-methylcyclohexane-1-carboxamide (4.66 g, 22.5 mmol, Intermediate 134). The flask was cooled to −78° C. and subsequently treated with DIBAL-H (23.6 mL, 23.6 mmol, 1 M in toluene) dropwise over 28 min. Once addition of DIBAL-H was complete, the mixture was stirred for an additional 4.5 h at −78° C., and then quenched via slow addition of 1 N aqueous HCl. The mixture was warmed to rt, diluted with EtOAc, and washed with 1 N aqueous HCl, water and brine. Combination of the aqueous washes resulted in the separation of an organic phase, which was combined with the EtOAc extracts, dried over anhydrous MgSO₄, filtered, and concentrated to dryness to afford the title compound.

### Intermediate 136

### (R)—N-((E)-((R)-3,3-Difluorocyclohexyl)methylene)-2-methylpropane-2-sulfinamide

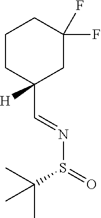 [SMILES: CC(C)(C)[S@@](=O)/N=C/[C@@H]1CCCC(F)(F)C1]

(R)-3,3-Difluorocyclohexane-1-carbaldehyde (2.92 g, 19.7 mmol, Intermediate 135), (R)-2-methylpropane-2-sulfinamide (2.69 g, 22.2 mmol), CuSO₄ (9.45 g, 59.2 mmol), pyridinium p-toluenesulfonate (504 mg, 2.01 mmol) and DCM (200 mL) were added to a 500 mL round-bottomed flask, and the resultant mixture stirred at rt for 67.3 h. After that time, the mixture was filtered through diatomaceous earth, concentrated to dryness, and the resultant residue purified by silica gel chromatography (0-100% EtOAc/hexanes). The purified material was dissolved in toluene (˜60 mL) and concentrated to dryness to afford the title compound as a white crystalline solid.

### Intermediate 137

### (S)—N-Methoxy-N-methyl-3-oxocyclohexane-1-carboxamide

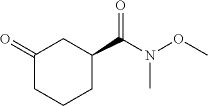

The title compound was prepared as described for the synthesis of Intermediate 133, using (1S)-3-oxo-cyclohexanecarboxylic acid in place of (1R)-3-oxo-cyclohexanecarboxylic acid, to provide the title compound.

### Intermediate 138

### (S)-3,3-Difluoro-N-methoxy-N-methylcyclohexane-1-carboxamide

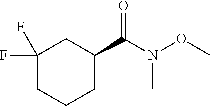

The title compound was prepared as described for the synthesis of Intermediate 134, using (S)—N-methoxy-N-methyl-3-oxocyclohexane-1-carboxamide (Intermediate 137) in place of (R)—N-methoxy-N-methyl-3-oxocyclohexane-1-carboxamide, to provide the title compound.

### Intermediate 139

### (S)-3,3-Difluorocyclohexane-1-carbaldehyde

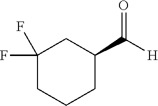

The title compound was prepared as described for the synthesis of Intermediate 135, using (S)-3,3-difluoro-N-methoxy-N-methylcyclohexane-1-carboxamide (Intermediate 138) in place of (R)-3,3-difluoro-N-methoxy-N-methylcyclohexane-1-carboxamide, to provide the title compound.

### Intermediate 140

### (R)—N-((E)-((S)-3,3-Difluorocyclohexyl)methylene)-2-methylpropane-2-sulfinamide

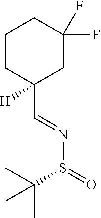 [SMILES: CC(C)(C)[S@@](=O)/N=C/[C@H]1CCCC(F)(F)C1]

The title compound was prepared as described for the synthesis of Intermediate 136, using (S)-3,3-difluorocyclohexane-1-carbaldehyde (Intermediate 139) in place of (R)-3,3-difluorocyclohexane-1-carbaldehyde, to provide the title compound as a white crystalline solid.

### Intermediate 141

### 4,4,4-Trifluoro-3-methyl-N—((R)-1-(1-((2-(trimethylsilyl)ethoxy)methyl)-1H-benzo[d]imidazol-6-yl)ethyl)butanamide and 4,4,4-trifluoro-3-methyl-N—((R)-1-(1-((2-(trimethylsilyl)ethoxy)methyl)-1H-benzo[d]imidazol-5-yl)ethyl)butanamide

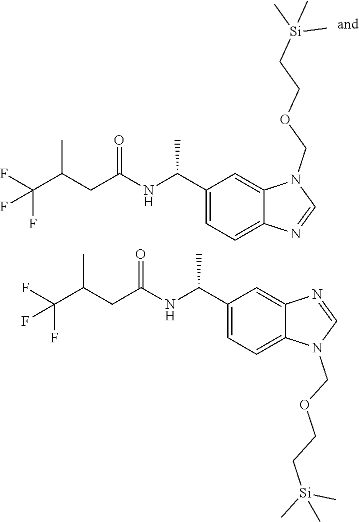

A flask was charged with (R)-1-(1-((2-(trimethylsilyl)ethoxy)methyl)-1H-benzo[d]imidazol-6-yl)ethan-1-amine and (R)-1-(1-((2-(trimethylsilyl)ethoxy)methyl)-1H-benzo[d]imidazol-5-yl)ethan-1-amine (2914 mg, 10 mmol, Intermediate 182), DMF (30 mL), HATU (4563 mg, 12 mmol), 3-trifluoromethylbutryric acid (1873 mg, 12 mmol) and DIPEA (4.3 mL, 25 mmol), and the resulting mixture was stirred for 15 min at rt. The reaction was quenched by the addition of water and the resulting suspension was extracted with EtOAc (3×30 mL). The combined organic extracts were washed with 10% aqueous LiCl followed by brine, dried over anhydrous MgSO₄, filtered. and condensed into an oil. The crude material was purified by silica gel chromatography (0-100% EtOAc/(10% MeOH in hexanes)). The product containing fractions were condensed into an off-white foam. The solid contained trace DMF and was dissolved in diethyl ether and washed with 10% aqueous LiCl, water and brine, dried over anhydrous MgSO₄, filtered. and condensed to afford the title compound as a yellow solid.

### Intermediate 142

### N—((R)-1-(2-((S)—(((R)-tert-Butylsulfinyl)amino)(4,4-difluorocyclohexyl)methyl)-1-((2-(trimethylsilyl)ethoxy)methyl)-1H-benzo[d]imidazol-5-yl)ethyl)-4,4,4-trifluoro-3-methylbutanamide and N—((R)-1-(2-((S)—(((R)-tert-butylsulfinyl)amino)(4,4-difluorocyclohexyl)methyl)-1-((2-(trimethylsilyl)ethoxy)methyl)-1H-benzo[d]imidazol-6-yl)ethyl)-4,4,4-trifluoro-3-methylbutanamide

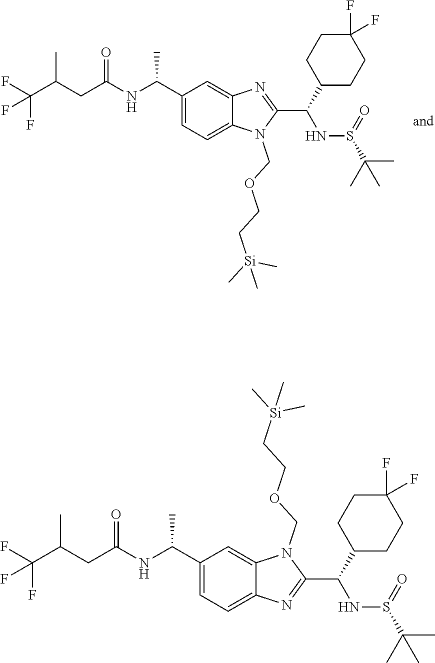 [SMILES: CC(CC(=O)N[C@H](C)c1ccc2c(c1)nc([C@@H](N[S@](=O)C(C)(C)C)C1CCC(F)(F)CC1)n2COCC[Si](C)(C)C)C(F)(F)F ; CC(CC(=O)N[C@H](C)c1ccc2nc([C@@H](N[S@](=O)C(C)(C)C)C3CCC(F)(F)CC3)n(COCC[Si](C)(C)C)c2c1)C(F)(F)F]

A flask was charged with 4,4,4-trifluoro-3-methyl-N—((R)-1-(1-((2-(trimethylsilyl)ethoxy)methyl)-1H-benzo[d]imidazol-6-yl)ethyl)butanamide and 4,4,4-trifluoro-3-methyl-N—((R)-1-(1-((2-(trimethylsilyl)ethoxy)methyl)-1H-benzo[d]imidazol-5-yl)ethyl)butanamide (2.74 g, 6.4 mmol, Intermediate 141) and THF (89 mL), and the mixture was cooled to −78° C. To the cold solution was added n-BuLi (6.7 mL, 13.4 mmol, 2 M in hexanes) and the reaction was stirred for 30 min at −78° C. To the solution was added (R,Z)—N-((4,4-difluorocyclohexyl)methylene)-2-methylpropane-2-sulfinamide (1923 mg, 7.7 mmol, Intermediate 234) and an additional 10 mL of THF. The reaction was stirred for 20 min then allowed to warm to rt and quenched by the slow addition of saturated aqueous NH₄Cl. The suspension was further diluted with water and extracted with EtOAc (3×30 mL). The combined organic extracts were washed with brine, dried over anhydrous MgSO₄, filtered and condensed. The crude material was purified by silica gel chromatography (0-100% EtOAc/hexanes). The product containing fractions were condensed to afford the title compound as an off-white foam.

### Intermediate 143

### (S*)—N—((R)-1-(2-((S)—(((R)-tert-Butylsulfinyl)amino)(4,4-difluorocyclohexyl)methyl)-1H-benzo[d]imidazol-5-yl)ethyl)-4,4,4-trifluoro-3-methylbutanamide

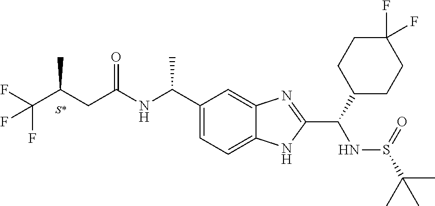 [SMILES: C[C@@H](NC(=O)C[C@H](C)C(F)(F)F)c1ccc2[nH]c([C@@H](N[S@](=O)C(C)(C)C)C3CCC(F)(F)CC3)nc2c1]

### Intermediate 144

### (R*)—N—((R)-1-(2-((S)—(((R)-tert-Butylsulfinyl)amino)(4,4-difluorocyclohexyl)methyl)-1H-benzo[d]imidazol-5-yl)ethyl)-4,4,4-trifluoro-3-methylbutanamide

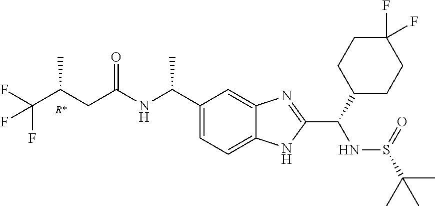 [SMILES: C[C@H](CC(=O)N[C@H](C)c1ccc2[nH]c([C@@H](N[S@](=O)C(C)(C)C)C3CCC(F)(F)CC3)nc2c1)C(F)(F)F]

A steel pressure bomb was charged with N—((R)-1-(2-((S)—(((R)-tert-butylsulfinyl)amino)(4,4-difluorocyclohexyl)methyl)-1-((2-(trimethylsilyl)ethoxy)methyl)-1H-benzo[d]imidazol-5-yl)ethyl)-4,4,4-trifluoro-3-methylbutanamide and N—((R)-1-(2-((S)—(((R)-tert-butylsulfinyl)amino)(4,4-difluorocyclohexyl)methyl)-1-((2-(trimethylsilyl)ethoxy)methyl)-1H-benzo[d]imidazol-6-yl)ethyl)-4,4,4-trifluoro-3-methylbutanamide (2.9 g, 4.3 mmol, Intermediate 142), THF (20 mL) and TBAF (10.6 mL, 10.6 mmol, 1 M in THF). The vessel was sealed and warmed to 90° C. and stirred overnight. The pressure bomb was cooled to rt and vented. The reaction mixture was poured over water and extracted with EtOAc (3×30 mL). The combined organics were washed with brine, dried over MgSO₄, filtered and condensed. The crude material was purified by silica gel chromatography (0-100% EtOAc/hexanes). The product containing fractions were condensed into a pale-yellow foam. The diastereomers were resolved by chiral SFC separation (Stationary phase AS-H, 15% isopropanol (with 0.1% diethylamine)/CO₂)). The first eluting fraction was Intermediate 143 and the second eluting fraction was Intermediate 144.

### Intermediate 145

### (S*)—N—((R)-1-(2-((S)-Amino(4,4-difluorocyclohexyl)methyl)-1H-benzo[d]imidazol-5-yl)ethyl)-4,4,4-trifluoro-3-methylbutanamide

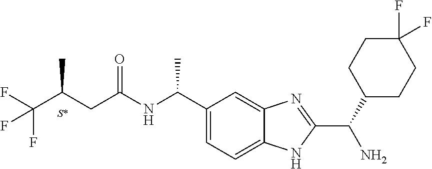 [SMILES: C[C@@H](NC(=O)C[C@H](C)C(F)(F)F)c1ccc2[nH]c([C@@H](N)C3CCC(F)(F)CC3)nc2c1]

A flask was charged with (S*)—N—((R)-1-(2-((S)—(((R)-tert-butylsulfinyl)amino)(4,4-difluorocyclohexyl)methyl)-1H-benzo[d]imidazol-5-yl)ethyl)-4,4,4-trifluoro-3-methylbutanamide (794 mg, 1.4 mmol, Intermediate 143), 1,4-dioxane (4.5 mL) and HCl (1.1 mL, 4.3 mmol, 4 M in dioxane). The reaction was stirred at rt for 1 h. The reaction was condensed into a yellow oil and then dissolved in water. The aqueous solution was washed with hexanes (2×15 mL, wash discarded). The remaining aqueous solution was made basic by the addition of 1 N aqueous NaOH and extracted with EtOAc (3×15 mL). The combined organic extracts were washed with brine, dried over anhydrous MgSO₄, filtered, and condensed to afford the title compound as a yellow foam.

### Intermediate 146

### (R*)—N—((R)-1-(2-((S)-Amino(4,4-difluorocyclohexyl)methyl)-1H-benzo[d]imidazol-5-yl)ethyl)-4,4,4-trifluoro-3-methylbutanamide

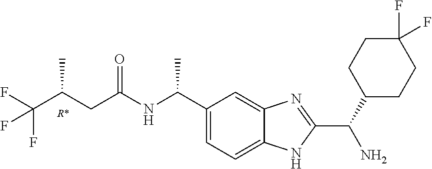 [SMILES: C[C@H](CC(=O)N[C@H](C)c1ccc2[nH]c([C@@H](N)C3CCC(F)(F)CC3)nc2c1)C(F)(F)F]

The title compound was prepared as described for Intermediate 145, using (R*)—N—((R)-1-(2-((S)—(((R)-tert-butylsulfinyl)amino)(4,4-difluorocyclohexyl)methyl)-1H-benzo[d]imidazol-5-yl)ethyl)-4,4,4-trifluoro-3-methylbutanamide (Intermediate 144) in place of (S*)—N—((R)-1-(2-((S)—(((R)-tert-butylsulfinyl)amino)(4,4-difluorocyclohexyl)methyl)-1H-benzo[d]imidazol-5-yl)ethyl)-4,4,4-trifluoro-3-methylbutanamide to afford the title compound as a yellow foam.

### Intermediate 147

### (R)-4,4,4-Trifluoro-N-(1-(1-((2-(trimethylsilyl)ethoxy)methyl)-1H-benzo[d]imidazol-6-yl)ethyl)butanamide and (R)-4,4,4-trifluoro-N-(1-(1-((2-(trimethylsilyl)ethoxy)methyl)-1H-benzo[d]imidazol-5-yl)ethyl)butanamide

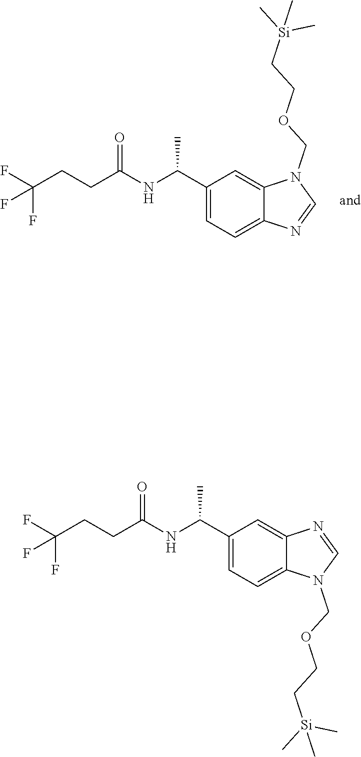 [SMILES: C[C@@H](NC(=O)CCC(F)(F)F)c1ccc2ncn(COCC[Si](C)(C)C)c2c1 ; C[C@@H](NC(=O)CCC(F)(F)F)c1ccc2c(c1)ncn2COCC[Si](C)(C)C]

A vial was charged with 4,4,4-trifluorobutyric acid (292 mg, 2.1 mmol), DMF (5 mL), bis(2-oxo-3-oxazolidinyl)phosphinic chloride (524 mg, 2.1 mmol), DIPEA and a mixture of (R)-1-(1-((2-(trimethylsilyl)ethoxy)methyl)-1H-benzo[d]imidazol-6-yl)ethan-1-amine and (R)-1-(1-((2-(trimethylsilyl)ethoxy)methyl)-1H-benzo[d]imidazol-5-yl)ethan-1-amine (500 mg, 1.8 mmol, Intermediate 182). The reaction was stirred at rt for 1 h. After that time, the mixture was poured over water and extracted with EtOAc (3×15 mL). The combined organic extracts were washed with 10% aqueous LiCl, water and brine, dried over MgSO₄, filtered and concentrated. The crude material was purified by silica gel chromatography (0-100% (10% MeOH in EtOAc)/hexanes) to afford the title compound as a foam.

### Intermediate 148

### N—((R)-1-(2-((S)—(((R)-tert-Butylsulfinyl)amino)(4,4-difluorocyclohexyl)methyl)-1-((2-(trimethylsilyl)ethoxy)methyl)-1H-benzo[d]imidazol-6-yl)ethyl)-4,4,4-trifluorobutanamide and N—((R)-1-(2-((S)—(((R)-tert-butylsulfinyl)amino)(4,4-difluorocyclohexyl)methyl)-1-((2-(trimethylsilyl)ethoxy)methyl)-1H-benzo[d]imidazol-5-yl)ethyl)-4,4,4-trifluorobutanamide

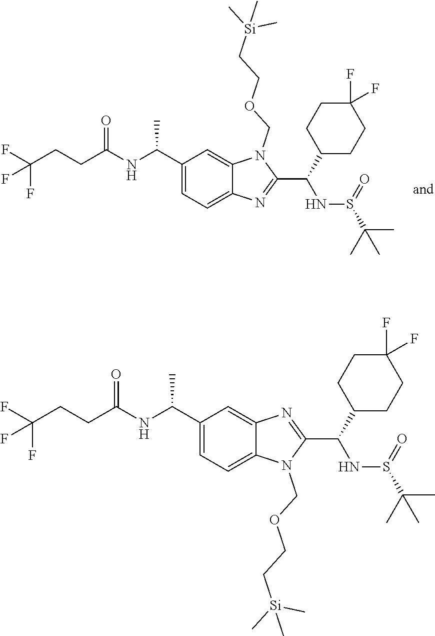 [SMILES: C[C@@H](NC(=O)CCC(F)(F)F)c1ccc2nc([C@@H](N[S@](=O)C(C)(C)C)C3CCC(F)(F)CC3)n(COCC[Si](C)(C)C)c2c1 ; C[C@@H](NC(=O)CCC(F)(F)F)c1ccc2c(c1)nc([C@@H](N[S@](=O)C(C)(C)C)C1CCC(F)(F)CC1)n2COCC[Si](C)(C)C]

An oven dried flask was placed under a nitrogen atmosphere. To the flask were added (R)-4,4,4-trifluoro-N-(1-(1-((2-(trimethylsilyl)ethoxy)methyl)-1H-benzo[d]imidazol-6-yl)ethyl)butanamide and (R)-4,4,4-trifluoro-N-(1-(1-((2-(trimethylsilyl)ethoxy)methyl)-1H-benzo[d]imidazol-5-yl)ethyl)butanamide (411 mg, 1.0 mmol, Intermediate 147), and THF (14 mL). The solution was cooled to −78° C. and n-BuLi (1.15 mL, 2.1 mmol, 2.5 M in hexanes) was added dropwise. The reaction was stirred for 10 min at −78° C. then a solution of (R,Z)—N-((4,4-difluorocyclohexyl)methylene)-2-methylpropane-2-sulfinamide (300 mg, 1.2 mmol, Intermediate 234) in THF (2 mL) was added dropwise. The reaction was warmed to rt over 30 min and then was quenched by the careful addition of saturated aqueous ammonium chloride. The mixture was further diluted with water and EtOAc. The layers were separated and the aqueous phase was further extracted with EtOAc (2×10 mL). The combined organics were washed with water and brine, dried over anhydrous MgSO₄, filtered and concentrated. The crude material was purified by silica gel chromatography (0-100% (10% MeOH in EtOAc)/hexanes) to afford the title compound as a glassy solid.

### Intermediate 149

### N—((R)-1-(2-((S)-Amino(4,4-difluorocyclohexyl)methyl)-1-((2-(trimethylsilyl)ethoxy)methyl)-1H-benzo[d]imidazol-6-yl)ethyl)-4,4,4-trifluorobutanamide and N—((R)-1-(2-((S)-amino(4,4-difluorocyclohexyl)methyl)-1-((2-(trimethylsilyl)ethoxy)methyl)-1H-benzo[d]imidazol-5-yl)ethyl)-4,4,4-trifluorobutanamide

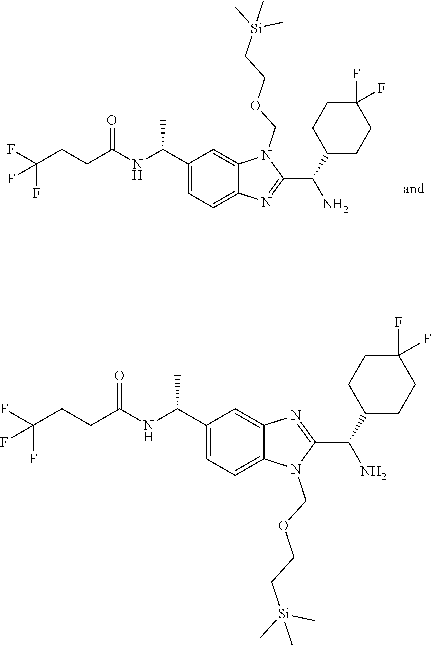 [SMILES: C[C@@H](NC(=O)CCC(F)(F)F)c1ccc2nc([C@@H](N)C3CCC(F)(F)CC3)n(COCC[Si](C)(C)C)c2c1 ; C[C@@H](NC(=O)CCC(F)(F)F)c1ccc2c(c1)nc([C@@H](N)C1CCC(F)(F)CC1)n2COCC[Si](C)(C)C]

A flask was charged with N—((R)-1-(2-((S)—(((R)-tert-butylsulfinyl)amino)(4,4-difluorocyclohexyl)methyl)-1-((2-(trimethylsilyl)ethoxy)methyl)-1H-benzo[d]imidazol-6-yl)ethyl)-4,4,4-trifluorobutanamide and N—((R)-1-(2-((S)—(((R)-tert-butylsulfinyl)amino)(4,4-difluorocyclohexyl)methyl)-1-((2-(trimethylsilyl)ethoxy)methyl)-1H-benzo[d]imidazol-5-yl)ethyl)-4,4,4-trifluorobutanamide (400 mg, 0.6 mmol, Intermediate 148), 1,4-dioxane (5 mL) and HCl (0.75 mL, 3 mmol, 4 M in 1,4-dioxane). The reaction was stirred at rt for 1 h. After that time, the volatiles were removed and the residue was dissolved in water and washed with hexanes (2×5 mL, wash discarded). The remaining aqueous phase was made basic by the addition of 1 N aqueous NaOH and extracted with EtOAc (3×5 mL). The combined organics were washed with brine, dried over anhydrous MgSO₄, filtered and condensed. The crude material was purified by silica gel chromatography (0-100% (10% MeOH in EtOAc)/hexanes) to afford the title compound as a white foam.

### Intermediate 150

### N—((S)-(4,4-Difluorocyclohexyl)(6-((R)-1-(4,4,4-trifluorobutanamido)ethyl)-1-((2-(trimethylsilyl)ethoxy)methyl)-1H-benzo[d]imidazol-2-yl)methyl)benzamide and N—((S)-(4,4-difluorocyclohexyl)(5-((R)-1-(4,4,4-trifluorobutanamido)ethyl)-1-((2-(trimethylsilyl)ethoxy)methyl)-1H-benzo[d]imidazol-2-yl)methyl)benzamide

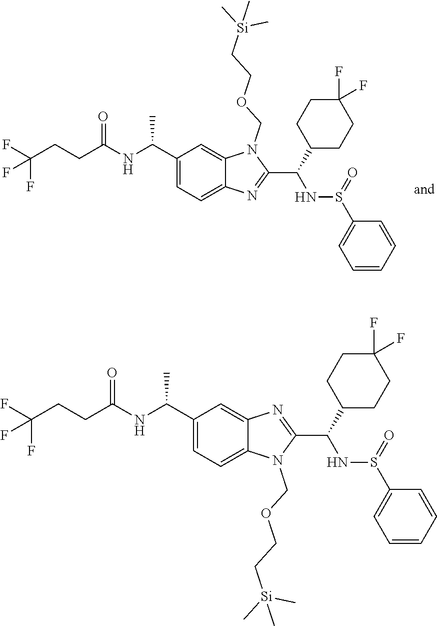 [SMILES: C[C@@H](NC(=O)CCC(F)(F)F)c1ccc2nc([C@@H](NS(=O)c3ccccc3)C3CCC(F)(F)CC3)n(COCC[Si](C)(C)C)c2c1 ; C[C@@H](NC(=O)CCC(F)(F)F)c1ccc2c(c1)nc([C@@H](NS(=O)c1ccccc1)C1CCC(F)(F)CC1)n2COCC[Si](C)(C)C]

A vial was charged with benzoic acid (24 mg, 0.19 mmol), DMF (2 mL), bis(2-oxo-3-oxazolidinyl)phosphinic chloride (50 mg, 0.19 mmol) and DIPEA (64 μL, 0.37 mmol). The reaction was stirred at rt for 5 min then N—((R)-1-(2-((S)-amino(4,4-difluorocyclohexyl)methyl)-1-((2-(trimethylsilyl)ethoxy)methyl)-1H-benzo[d]imidazol-6-yl)ethyl)-4,4,4-trifluorobutanamide and N—((R)-1-(2-((S)-amino(4,4-difluorocyclohexyl)methyl)-1-((2-(trimethylsilyl)ethoxy)methyl)-1H-benzo[d]imidazol-5-yl)ethyl)-4,4,4-trifluorobutanamide (83 mg, 0.15 mmol, Intermediate 149) was added and the mixture was stirred at rt for 15 min. After that time, the reaction was poured over water and extracted with EtOAc (3×5 mL). The combined organics were washed with 10% aqueous LiCl, water and brine, dried over anhydrous MgSO₄, filtered and concentrated. The crude material was purified by silica gel chromatography (0-100% (10% MeOH in EtOAc)/hexanes) to afford the title compound as a white solid.

### Intermediate 151

### N—((S)-1-(2-((S)—(((R)-tert-Butylsulfinyl)amino)(4,4-difluorocyclohexyl)methyl)-1H-benzo[d]imidazol-5-yl)ethyl)-4,4,4-trifluorobutanamide

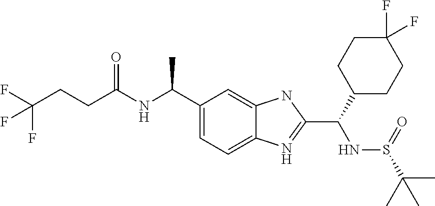 [SMILES: C[C@H](NC(=O)CCC(F)(F)F)c1ccc2nc([C@@H](N[S@](=O)C(C)(C)C)C3CCC(F)(F)CC3)[nH]c2c1]

The title compound was prepared as described for Intermediate 153, using (R)—N—((S)-(5-((S)-1-aminoethyl)-1H-benzo[d]imidazol-2-yl)(4,4-difluorocyclohexyl)methyl)-2-methylpropane-2-sulfinamide (Intermediate 2) in place of (R)—N—((S)-(5-((R)-1-aminoethyl)-1H-benzo[d]imidazol-2-yl)(4,4-difluorocyclohexyl)methyl)-2-methylpropane-2-sulfinamide and 4,4,4-trifluorobutanoic acid in place of 4,4,4-trifluoro-3-(trifluoromethyl)butanoic acid to afford the title compound as an off-white foam.

### Intermediate 152

### N—((S)-1-(2-((S)-Amino(4,4-difluorocyclohexyl)methyl)-1H-benzo[d]imidazol-5-yl)ethyl)-4,4,4-trifluorobutanamide

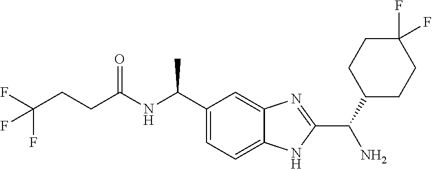 [SMILES: C[C@H](NC(=O)CCC(F)(F)F)c1ccc2[nH]c([C@@H](N)C3CCC(F)(F)CC3)nc2c1]

The title compound was prepared as described for Intermediate 4, using N—((S)-1-(2-((S)—(((R)-tert-butylsulfinyl)amino)(4,4-difluorocyclohexyl)methyl)-1H-benzo[d]imidazol-5-yl)ethyl)-4,4,4-trifluorobutanamide (Intermediate 151) in place of N—((R)-1-(2-((S)—(((R)-tert-butylsulfinyl)amino)(4,4-difluorocyclohexyl)methyl)-1H-benzo[d]imidazol-5-yl)ethyl)-4,4,4-trifluorobutanamide to afford the title compound as a white solid.

### Intermediate 153

### N—((R)-1-(2-((S)—(((R)-tert-Butylsulfinyl)amino)(4,4-difluorocyclohexyl)methyl)-1H-benzo[d]imidazol-6-yl)ethyl)-4,4,4-trifluoro-3-(trifluoromethyl)butanamide

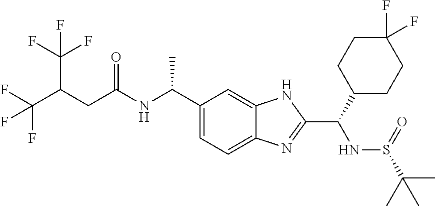 [SMILES: C[C@@H](NC(=O)CC(C(F)(F)F)C(F)(F)F)c1ccc2nc([C@@H](N[S@](=O)C(C)(C)C)C3CCC(F)(F)CC3)[nH]c2c1]

A vial was charged with 4,4,4-trifluoro-3-(trifluoromethyl)butanoic acid (486 mg, 2.3 mmol), HATU (880 mg, 2.3 mmol) and DMF (10 mL). The reaction was stirred at rt for 5 min before (R)—N—((S)-(5-((R)-1-aminoethyl)-1H-benzo[d]imidazol-2-yl)(4,4-difluorocyclohexyl)methyl)-2-methylpropane-2-sulfinamide (734 mg, 1.8 mmol, Intermediate 1) and DIPEA (0.61 mL, 3.6 mmol) were added and the reaction was stirred at rt for an additional 15 min. The reaction was poured over water and extracted with EtOAc (2×10 mL). The combined organics were washed with 10% aqueous LiCl and brine, dried over anhydrous MgSO₄, filtered and concentrated. The crude material was purified by silica gel chromatography (0-100% EtOAc (with 10% MeOH)/hexanes) to afford the title compound as a yellow foam.

### Intermediate 154

### N—((R)-1-(2-((S)-Amino(4,4-difluorocyclohexyl)methyl)-1H-benzo[d]imidazol-6-yl)ethyl)-4,4,4-trifluoro-3-(trifluoromethyl)butanamide

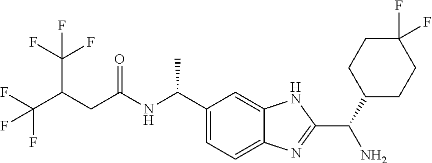 [SMILES: C[C@@H](NC(=O)CC(C(F)(F)F)C(F)(F)F)c1ccc2nc([C@@H](N)C3CCC(F)(F)CC3)[nH]c2c1]

The title compound was prepared as described for Intermediate 145, using N—((R)-1-(2-((S)—(((R)-tert-butylsulfinyl)amino)(4,4-difluorocyclohexyl)methyl)-1H-benzo[d]imidazol-6-yl)ethyl)-4,4,4-trifluoro-3-(trifluoromethyl)butanamide (Intermediate 153) in place of (S*)—N—((R)-1-(2-((S)—(((R)-tert-butylsulfinyl)amino)(4,4-difluorocyclohexyl)methyl)-1H-benzo[d]imidazol-5-yl)ethyl)-4,4,4-trifluoro-3-methylbutanamide to afford the title compound as an off white foam.

### Intermediate 155

### N—((S)-(6-((R)-1-Aminoethyl)-1H-benzo[d]imidazol-2-yl)(4,4-difluorocyclohexyl)methyl)-1-methyl-1H-pyrazole-5-carboxamide

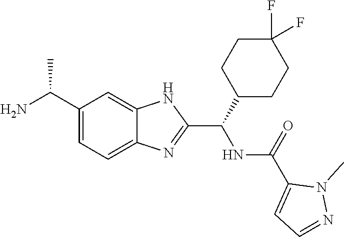 [SMILES: C[C@@H](N)c1ccc2nc([C@@H](NC(=O)c3ccnn3C)C3CCC(F)(F)CC3)[nH]c2c1]

A vial was charged with N—((S)-(5-((R)-1-(((S)-tert-butylsulfinyl)amino)ethyl)-1H-benzo[d]imidazol-2-yl)(4,4-difluorocyclohexyl)methyl)-1-methyl-1H-pyrazole-5-carboxamide (500 mg, 0.96 mmol, Intermediate247), 1,4-dioxane (3 mL) and HCl (0.72 mL, 2.88 mmol, 4 M in 1,4-dioxane). The reaction was stirred at rt for 30 min. After that time, the mixture was condensed and the residue was dissolved in water and washed with diethyl ether (3×5 mL, wash discarded). The remaining aqueous layer was made basic by the addition of 1 N aqueous NaOH and extracted with EtOAc (3×10 mL). The combined organic layers were washed with brine, dried over anhydrous MgSO₄, filtered, and concentrated to afford the title compound as an off-white foam.

### Intermediate 156

### (R)—N—((S)-(5-((S)-Amino(cyclopropyl)methyl)-1H-benzo[d]imidazol-2-yl)(4,4-difluorocyclohexyl)methyl)-2-methylpropane-2-sulfinamide

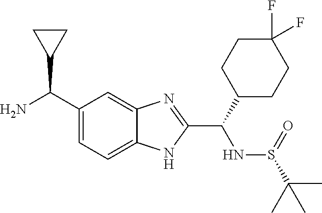 [SMILES: CC(C)(C)[S@@](=O)N[C@H](c1nc2cc([C@@H](N)C3CC3)ccc2[nH]1)C1CCC(F)(F)CC1]

A solution of (R)—N—((S)-(5-cyano-1H-benzo[d]imidazol-2-yl)(4,4-difluorocyclohexyl)methyl)-2-methylpropane-2-sulfinamide (30 g, 76 mmol, Intermediate 62) in THF (300 mL) was cooled to 0° C. and then cyclopropylmagnesium bromide (1520 mL, 760 mmol, 0.5 M in THF) was added dropwise and the resulting solution was stirred at 6° C. for 12 h. After that time, the solution was cooled to 0° C. and MeOH (300 mL) was added dropwise. The solution was stirred at 0° C. for 1.5 h and then NaBH₄ (3.45 g, 91.2 mmol) was added and the mixture was stirred at 15° C. for 2 h. Seven similar scale batches were set up in parallel and combined for workup. Then, the reaction was quenched with water (250 mL), additional water (800 mL) was added and the mixture was extracted with DCM (4 L). The organic layer was washed with brine (600 mL), dried over anhydrous MgSO₄, filtered, and concentrated to dryness. The residue was purified by silica gel chromatography (2-50% MeOH/DCM) followed by SFC (DAICEL CHIRALPAK AD, 250×50 mm, 10 μm, mobile phase: 40% CO₂ in EtOH (0.1% NH₄OH)) to provide the title compound (second eluting isomer) as a white solid.

### Intermediate 157

### (R)—N—((S)-(5-((R)-Amino(cyclopropyl)methyl)-1H-benzo[d]imidazol-2-yl)(4,4-difluorocyclohexyl)methyl)-2-methylpropane-2-sulfinamide

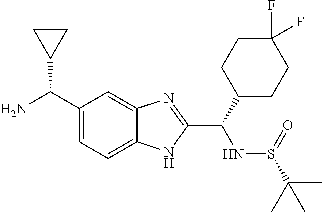 [SMILES: CC(C)(C)[S@@](=O)N[C@H](c1nc2cc([C@H](N)C3CC3)ccc2[nH]1)C1CCC(F)(F)CC1]

The title compound was prepared as described for the synthesis of Intermediate 156, and was further purified by SFC (DAICEL CHIRALCEL OD, 250×50 mm, 10 μm, mobile phase: 25% CO₂ in EtOH (0.1% NH₄OH)) followed by preparative HPLC (Phenomenex luna, C18, 250×80 mm, 10 μm, mobile phase: 20-50% ACN/water (with 10 mM NH₄HCO₃)) to provide the title compound as a white solid.

### Intermediate 158

### N—((R)-(2-((S)—(((R)-tert-Butylsulfinyl)amino)(4,4-difluorocyclohexyl)methyl)-1H-benzo[d]imidazol-6-yl)(cyclopropyl)methyl)-2-(3,3-difluorocyclobutyl)acetamide

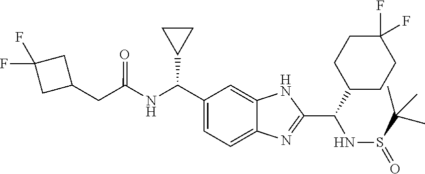 [SMILES: CC(C)(C)[S@@](=O)N[C@H](c1nc2ccc([C@H](NC(=O)CC3CC(F)(F)C3)C3CC3)cc2[nH]1)C1CCC(F)(F)CC1]

To a stirred solution of 2-(3,3-difluorocyclobutyl)acetic acid (462 mg, 3.08 mmol) and 1-propanephosphonic anhydride (2.04 mL, 3.42 mmol, 50% in EtOAc) in EtOAc (11.4 mL) at rt was added N,N-diisopropylethylamine (1.56 mL, 9.12 mmol). After 3 min, (R)—N—((S)-(5-((R)-amino(cyclopropyl)methyl)-1H-benzo[d]imidazol-2-yl)(4,4-difluorocyclohexyl)methyl)-2-methylpropane-2-sulfinamide (1.00 g, 2.28 mmol, Intermediate 157) and DCM (3.0 mL) were added. After stirring for 2 h at rt, additional portions of 2-(3,3-difluorocyclobutyl)acetic acid (100 mg, 0.67 mmol), 1-propanephosphonic anhydride (0.50 mL, 0.84 mmol, 50% in EtOAc) and N,N-diisopropylethylamine (0.50 mL, 2.91 mmol) were added. After stirring for 2 h at rt, the reaction mixture was diluted with water (25 mL) and EtOAc (25 mL). The aqueous portion was extracted with EtOAc (3×20 mL). The combined organic layers were washed with aqueous 0.1 M HCl/brine (10/1, 2×20 mL) then brine (20 mL), dried over anhydrous Na₂SO₄ and concentrated to provide the title compound as a gum.

### Intermediate 159

### N—((R)-(2-((S)-Amino(4,4-difluorocyclohexyl)methyl)-1H-benzo[d]imidazol-6-yl)(cyclopropyl)methyl)-2-(3,3-difluorocyclobutyl)acetamide hydrochloride

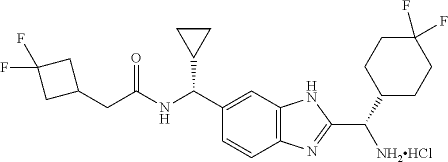

To a stirred suspension of N—((R)-(2-((S)—(((R)-tert-butylsulfinyl)amino)(4,4-difluorocyclohexyl)methyl)-1H-benzo[d]imidazol-6-yl)(cyclopropyl)methyl)-2-(3,3-difluorocyclobutyl)acetamide (crude material, maximum 2.28 mmol, Intermediate 158) in 1,4-dioxane (12 mL) and EtOAc (10 mL) was added HCl (4.56 mL, 18.2 mmol, 4 M in 1,4-dioxane) and the resulting mixture was stirred at rt for 3.5 h. After that time, the reaction mixture was diluted with hexanes (30 mL) and stirred for 5 min. The reaction mixture was filtered, and the solids were washed with hexanes (30 mL) and dried in vacuo to provide the title compound as a white powder.

### Intermediate 160

### N—((S)-(2-((S)—(((R)-tert-Butylsulfinyl)amino)(4,4-difluorocyclohexyl)methyl)-1H-benzo[d]imidazol-6-yl)(cyclopropyl)methyl)-2-(3,3-difluorocyclobutyl)acetamide

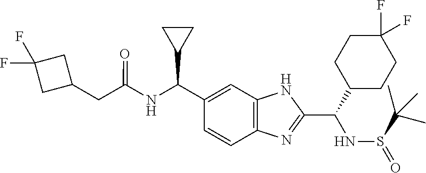 [SMILES: CC(C)(C)[S@@](=O)N[C@H](c1nc2ccc([C@@H](NC(=O)CC3CC(F)(F)C3)C3CC3)cc2[nH]1)C1CCC(F)(F)CC1]

The title compound was prepared as described for the synthesis of Intermediate 158, using (R)—N—((S)-(5-((S)-amino(cyclopropyl)methyl)-1H-benzo[d]imidazol-2-yl)(4,4-difluorocyclohexyl)methyl)-2-methylpropane-2-sulfinamide (Intermediate 156) in place of (R)—N—((S)-(5-((R)-amino(cyclopropyl)methyl)-1H-benzo[d]imidazol-2-yl)(4,4-difluorocyclohexyl)methyl)-2-methylpropane-2-sulfinamide.

### Intermediate 161

### N—((S)-(2-((S)-Amino(4,4-difluorocyclohexyl)methyl)-1H-benzo[d]imidazol-6-yl)(cyclopropyl)methyl)-2-(3,3-difluorocyclobutyl)acetamide hydrochloride

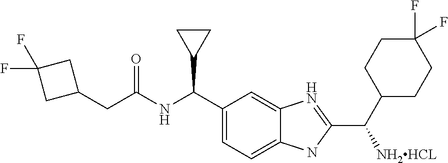

The title compound was prepared as described for the synthesis of Intermediate 159, using N—((S)-(2-((S)—(((R)-tert-butylsulfinyl)amino)(4,4-difluorocyclohexyl)methyl)-1H-benzo[d]imidazol-6-yl)(cyclopropyl)methyl)-2-(3,3-difluorocyclobutyl)acetamide (Intermediate 160) in place of N—((R)-(2-((S)—(((R)-tert-butylsulfinyl)amino)(4,4-difluorocyclohexyl)methyl)-1H-benzo[d]imidazol-6-yl)(cyclopropyl)methyl)-2-(3,3-difluorocyclobutyl)acetamide.

### Intermediate 162

### (R)—N—((S)-(5-((R)-Amino(cyclopropyl)methyl)-1-((2-(trimethylsilyl)ethoxy)methyl)-1H-benzo[d]imidazol-2-yl)(4,4-difluorocyclohexyl)methyl)-2-methylpropane-2-sulfinamide

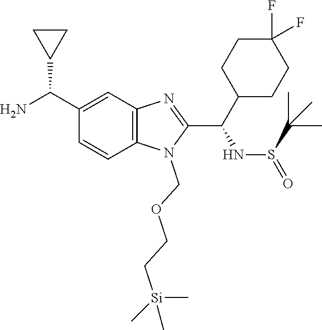 [SMILES: CC(C)(C)[S@@](=O)N[C@H](c1nc2cc([C@H](N)C3CC3)ccc2n1COCC[Si](C)(C)C)C1CCC(F)(F)CC1]

An oven-dried vial with a stir bar was charged with (R)-cyclopropyl(1-((2-(trimethylsilyl)ethoxy)methyl)-1H-benzo[d]imidazol-5-yl)methanamine hydrochloride (354 mg, 1.00 mmol, Intermediate 248 or 249) and THF (9.1 mL) under an N2 atmosphere. The reaction mixture was cooled to −78° C. and n-BuLi (1.04 mL, 2.60 mmol, 2.5 M in hexanes) was added in a dropwise manner over 2 min. After stirring for 30 min at −78° C., the reaction mixture was warmed to 0° C. for 5 min, then cooled to −78° C. and a solution of (R,Z)—N-((4,4-difluorocyclohexyl)methylene)-2-methylpropane-2-sulfinamide (352 mg, 1.40 mmol, Intermediate 234) in THF (1 mL) was added. After stirring for 2 h at −78° C., the reaction mixture was treated with EtOH (1 mL) and EtOAc (10 mL) and allowed to warm to rt. The reaction mixture was diluted with EtOAc (60 mL) and half saturated brine (60 mL), and the aqueous layer was extracted with EtOAc (2×50 mL). The combined organic layers were washed with brine (60 mL), dried over anhydrous Na₂SO₄, filtered and concentrated. The residue was purified by silica gel chromatography (100% EtOAc to 3/1 EtOAc/(EtOH+1% NH₄OH)) to provide the title compound.

### Intermediate 163

### N—((R)-(2-((S)—(((R)-tert-Butylsulfinyl)amino)(4,4-difluorocyclohexyl)methyl)-1-((2-(trimethylsilyl)ethoxy)methyl)-1H-benzo[d]imidazol-5-yl)(cyclopropyl)methyl)-3-cyclopropyl-2,2-difluoropropanamide

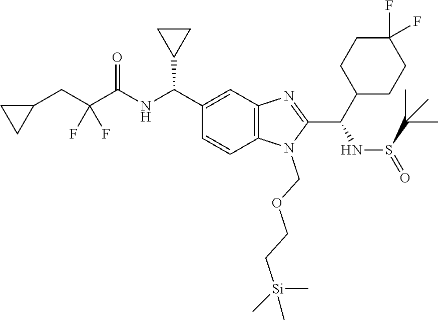 [SMILES: CC(C)(C)[S@@](=O)N[C@H](c1nc2cc([C@H](NC(=O)C(F)(F)CC3CC3)C3CC3)ccc2n1COCC[Si](C)(C)C)C1CCC(F)(F)CC1]

To a stirred solution of 3-cyclopropyl-2,2-difluoropropanoic acid (162 mg, 1.08 mmol), (R)—N—((S)-(5-((R)-amino(cyclopropyl)methyl)-1-((2-(trimethylsilyl)ethoxy)methyl)-1H-benzo[d]imidazol-2-yl)(4,4-difluorocyclohexyl)methyl)-2-methylpropane-2-sulfinamide (474 mg, 0.833 mmol, Intermediate 162) and 1-propanephosphonic anhydride (0.74 mL, 1.25 mmol, 50% in EtOAc) in DCM (4.2 mL) was added triethylamine (0.348 mL, 2.50 mmol). The resulting mixture was stirred at rt for 2 h, then additional portions of 3-cyclopropyl-2,2-difluoropropanoic acid (81 mg, 0.54 mmol), T3P® (0.35 mL, 0.59 mmol, 50% in EtOAc) and triethylamine (0.16 mL, 1.15 mmol) were added. After stirring at rt for an additional 6.5 h the reaction mixture was partitioned between water (40 mL) and EtOAc (40 mL). The organic layer was washed with aqueous HCl (40 mL, 0.1 M), water (20 mL) and brine (30 mL), and then dried over anhydrous Na₂SO₄, filtered, and concentrated to dryness. The residue was purified by silica gel chromatography (30-100% EtOAc/hexanes) to provide the title compound.

### Intermediate 164

### N—((R)-(2-((S)-Amino(4,4-difluorocyclohexyl)methyl)-1H-benzo[d]imidazol-5-yl)(cyclopropyl)methyl)-3-cyclopropyl-2,2-difluoropropanamide

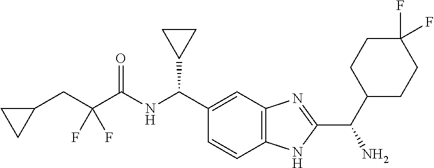 [SMILES: N[C@H](c1nc2cc([C@H](NC(=O)C(F)(F)CC3CC3)C3CC3)ccc2[nH]1)C1CCC(F)(F)CC1]

To a stirred solution of N—((R)-(2-((S)—(((R)-tert-butylsulfinyl)amino)(4,4-difluorocyclohexyl)methyl)-1-((2-(trimethylsilyl)ethoxy)methyl)-1H-benzo[d]imidazol-5-yl)(cyclopropyl)methyl)-3-cyclopropyl-2,2-difluoropropanamide (451 mg, 0.643 mmol, Intermediate 163) in 1,4-dioxane (6 mL) was added HCl (1.61 mL, 6.43 mmol, 4 M in 1,4-dioxane) and the resulting mixture was stirred at 55° C. for 4 h. Additional HCl (0.80 mL, 3.22 mmol, 4 M in 1,4-dioxane) and MeOH (0.5 mL) were then added and the reaction mixture was stirred at 55° C. for 3.5 h. After that time, the mixture was concentrated, the residue suspended in water (50 mL) and the aqueous layer was washed with hexanes (3×25 mL). The aqueous layer was basified to pH 10 with aqueous NaOH (3 M) and extracted with EtOAc (3×30 mL). The combined organics were dried over anhydrous Na₂SO₄, filtered, and concentrated to provide the title compound, which was used without further purification.

### Intermediate 165

### 6,6-Difluoro-N-methoxy-N-methylspiro[3.3]heptane-2-carboxamide

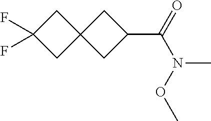

To a stirred solution of 6,6-difluorospiro[3.3]heptane-2-carboxylic acid (4.00 g, 22.7 mmol), N,O-dimethylhydroxylamine hydrochloride (2.66 g, 27.2 mmol) and HATU (10.36 g, 27.2 mmol) in DMF (76 mL) was added N,N-diisopropylethylamine (9.87 mL, 56.8 mmol). The mixture was stirred at rt for 1 h, and then additional portions of N,O-dimethylhydroxylamine hydrochloride (2.66 g, 27.2 mmol), HATU (10.36 g, 27.2 mmol) and N,N-diisopropylethylamine (9.87 mL, 56.8 mmol) were added. The mixture was stirred at rt for 16 h and then concentrated to remove DMF. The resulting mixture was partitioned between water (250 mL) and EtOAc (100 mL), and the aqueous layer was further extracted with EtOAc (100 mL). The combined organics were washed with water (2×100 mL) and brine (100 mL), dried over anhydrous Na₂SO₄, filtered, and concentrated to dryness. The residue was purified by silica gel chromatography (10-50% EtOAc/hexanes) to provide the title compound as an oil.

### Intermediate 166

### 6,6-Difluorospiro[3.3]heptane-2-carbaldehyde

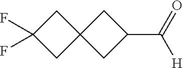 [SMILES: O=CC1CC2(C1)CC(F)(F)C2]

To a −78° C. solution of 6,6-difluoro-N-methoxy-N-methylspiro[3.3]heptane-2-carboxamide (3.50 g, 13.3 mmol, Intermediate 165) in DCM (66.5 mL) was added DIBAL-H (14.0 mL, 14.0 mmol, 1 M in toluene) in a dropwise manner over 5 min. The resulting mixture was stirred at −78° C. for 4 h. After that time, the reaction mixture was treated with water (6 mL) and saturated aqueous NH₄Cl (2 mL) and allowed to warm to rt and stir overnight. The reaction mixture was then filtered, dried over anhydrous MgSO₄, filtered, and concentrated to provide an oil that was used without further purification.

### Intermediate 167

### (E/Z)-2,2-Difluoro-6-(2-methoxyvinyl)spiro[3.3]heptane

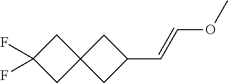

To a 0° C. suspension of (methoxymethyl)triphenylphosphonium chloride (5.87 g, 16.6 mmol) in THF (50 mL) was added sodium bis(trimethylsilyl)amide (15.3 mL, 15.3 mmol, 1 M in THF) in a dropwise manner over 5 min. The reaction mixture was stirred at 0° C. for 15 min then cooled to −78° C. and treated with a solution of crude 6,6-difluorospiro[3.3]heptane-2-carbaldehyde (theoretical 2.13 g, 13.3 mmol, Intermediate 166) in THF (25 mL) in a dropwise manner over 5 min. After 2 h at −78° C., the reaction mixture was warmed to rt and stirred for an additional 2 h. The reaction mixture was treated with saturated aqueous NH₄Cl (5 mL) and then partitioned between hexanes (75 mL) and saturated aqueous NaHCO₃ (100 mL). The aqueous layer was further extracted with 1/1 hexanes/EtOAc (50 mL). The combined organics were dried over anhydrous K₂CO₃, filtered, and concentrated. The residue was purified by silica gel chromatography (0-25% EtOAc/hexanes) to provide the title compound (3/1 E/Z ratio).

### Intermediate 168

### 2-(6,6-Difluorospiro[3.3]heptan-2-yl)acetaldehyde

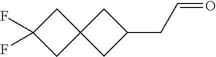 [SMILES: O=CCC1CC2(C1)CC(F)(F)C2]

To a stirred solution of (E/Z)-2,2-difluoro-6-(2-methoxyvinyl)spiro[3.3]heptane (1.04 g, 5.34 mmol, Intermediate 167) in acetone (35 mL) and water (3.9 mL) was added HCl (0.11 mL, 1.33 mmol, 37% in water) and the reaction mixture was heated to 65° C. After 1.5 h, the reaction mixture was cooled and diluted with brine (50 mL), saturated aqueous NaHCO₃ (50 mL) and hexanes (50 mL). The aqueous layer was extracted with DCM (3×50 mL). The combined organics were dried over anhydrous MgSO₄, filtered, and concentrated to provide an oil that was used without further purification.

### Intermediate 169

### (R,E)-N-(2-(6,6-Difluorospiro[3.3]heptan-2-yl)ethylidene)-2-methylpropane-2-sulfinamide

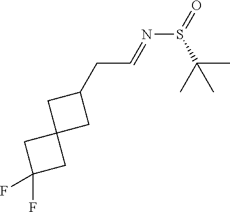 [SMILES: CC(C)(C)[S@@](=O)/N=C/CC1CC2(C1)CC(F)(F)C2]

To a stirred solution of 2-(6,6-difluorospiro[3.3]heptan-2-yl)acetaldehyde (929 mg, 5.33 mmol, Intermediate 168) in DCM (10.7 mL) was added (R)-(+)-2-methyl-2-propanesulfinamide (1.61 g, 13.3 mmol), copper (II) sulfate (5.11 g, 32.0 mmol) and PPTS (134 mg, 0.533 mmol). The reaction mixture was stirred at rt for 24 h, filtered and the solids washed with DCM (15 mL). The filtrate was concentrated to dryness and purified by silica gel chromatography (0-100% EtOAc/hexanes) to provide the title compound as a semisolid.

### Intermediate 170

### (R)—N—((S)-1-(5-((R)-Cyclopropyl(1,3-dioxoisoindolin-2-yl)methyl)-1-((2-(trimethylsilyl)ethoxy)methyl)-1H-benzo[d]imidazol-2-yl)-2-(6,6-difluorospiro[3.3]heptan-2-yl)ethyl)-2-methylpropane-2-sulfinamide

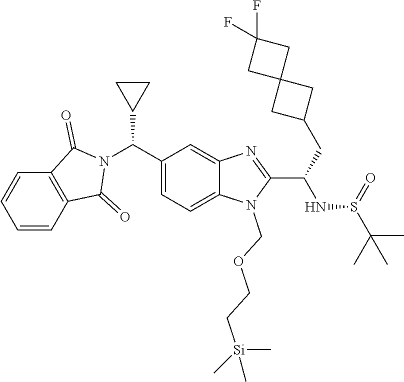 [SMILES: CC(C)(C)[S@@](=O)N[C@@H](CC1CC2(C1)CC(F)(F)C2)c1nc2cc([C@@H](C3CC3)N3C(=O)c4ccccc4C3=O)ccc2n1COCC[Si](C)(C)C]

An oven-dried vial was charged with (R)-2-(cyclopropyl(1-((2-(trimethylsilyl)ethoxy)methyl)-1H-benzo[d]imidazol-5-yl)methyl)isoindoline-1,3-dione (700 mg, 1.53 mmol, Intermediate 262), (R,E)-N-(2-(6,6-difluorospiro[3.3]heptan-2-yl)ethylidene)-2-methylpropane-2-sulfinamide (551 mg, 1.99 mmol, Intermediate 169) and THF (7.7 mL) under an N2 atmosphere. The reaction mixture was cooled to −78° C. and treated with LDA (2.75 mL, 2.75 mmol, 1 M in THF) in a dropwise manner over 2 min. After stirring at −78° C. for 1.5 h, the reaction mixture was treated with saturated aqueous NH₄Cl (3 mL) and allowed to warm to rt, at which time it was partitioned between water (40 mL) and EtOAc (20 mL). The aqueous layer was extracted with EtOAc (3×30 mL) and DCM (30 mL). The combined organics were dried over anhydrous Na₂SO₄, filtered, and concentrated to dryness. The residue was purified by silica gel chromatography (20-100% EtOAc/hexanes) to provide the title compound as a gum.

### Intermediate 171

### (R)—N—((S)-1-(5-((R)-Amino(cyclopropyl)methyl)-1-((2-(trimethylsilyl)ethoxy)methyl)-1H-benzo[d]imidazol-2-yl)-2-(6,6-difluorospiro[3.3]heptan-2-yl)ethyl)-2-methylpropane-2-sulfinamide

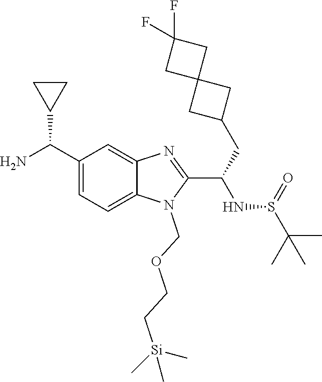 [SMILES: CC(C)(C)[S@@](=O)N[C@@H](CC1CC2(C1)CC(F)(F)C2)c1nc2cc([C@H](N)C3CC3)ccc2n1COCC[Si](C)(C)C]

To a stirred solution of (R)—N—((S)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-(6,6-difluorospiro[3.3]heptan-2-yl)ethyl)-2-methylpropane-2-sulfinamide (632 mg, 0.872 mmol, Intermediate 170) in EtOH (4.4 mL) was added hydrazine hydrate (86.3 mL, 1.74 mmol). After stirring for 1 h at rt, the reaction mixture was heated to 35° C. and stirred for an additional 3 h. The reaction mixture was then cooled to 0° C., filtered, and the solids washed with ice cold EtOH (10 mL). The filtrate was concentrated to dryness to provide the title compound as a foam that was used without further purification.

### Intermediate 172

### N—((R)-(2-((S)-1-(((R)-tert-Butylsulfinyl)amino)-2-(6,6-difluorospiro[3.3]heptan-2-yl)ethyl)-1-((2-(trimethylsilyl)ethoxy)methyl)-1H-benzo[d]imidazol-5-yl)(cyclopropyl)methyl)-2-(3,3-difluorocyclobutyl)acetamide

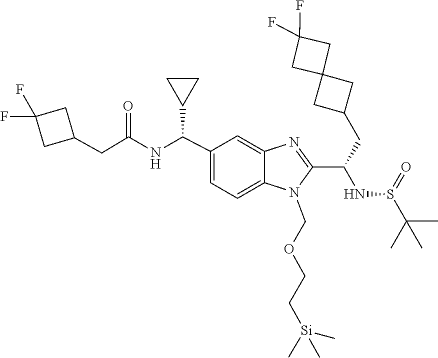 [SMILES: CC(C)(C)[S@@](=O)N[C@@H](CC1CC2(C1)CC(F)(F)C2)c1nc2cc([C@H](NC(=O)CC3CC(F)(F)C3)C3CC3)ccc2n1COCC[Si](C)(C)C]

The title compound was prepared as described for the synthesis of Intermediate 158, using (R)—N—((S)-1-(5-((R)-amino(cyclopropyl)methyl)-1-((2-(trimethylsilyl)ethoxy)methyl)-1H-benzo[d]imidazol-2-yl)-2-(6,6-difluorospiro[3.3]heptan-2-yl)ethyl)-2-methylpropane-2-sulfinamide (Intermediate 171) in place of (R)—N—((S)-(5-((R)-amino(cyclopropyl)methyl)-1H-benzo[d]imidazol-2-yl)(4,4-difluorocyclohexyl)methyl)-2-methylpropane-2-sulfinamide, to provide the title compound as a gum.

### Intermediate 173

### N—((R)-(2-((S)-1-Amino-2-(6,6-difluorospiro[3.3]heptan-2-yl)ethyl)-1H-benzo[d]imidazol-6-yl)(cyclopropyl)methyl)-2-(3,3-difluorocyclobutyl)acetamide

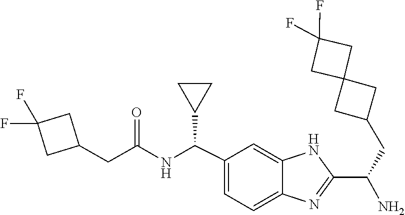 [SMILES: N[C@@H](CC1CC2(C1)CC(F)(F)C2)c1nc2ccc([C@H](NC(=O)CC3CC(F)(F)C3)C3CC3)cc2[nH]1]

The title compound was prepared as described for the synthesis of Intermediate 159, using N—((R)-(2-((S)-1-(((R)-tert-butylsulfinyl)amino)-2-(6,6-difluorospiro[3.3]heptan-2-yl)ethyl)-1-((2-(trimethylsilyl)ethoxy)methyl)-1H-benzo[d]imidazol-5-yl)(cyclopropyl)methyl)-2-(3,3-difluorocyclobutyl)acetamide (Intermediate 172), and neutralizing the mixture with aqueous 3 M NaOH to isolate the free amine as a gum that was used without further purification.

### Intermediate 174

### 3-((1,1,1-Trifluoro-2-methylpropan-2-yl)oxy)prop-1-ene

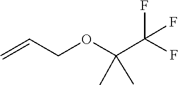

To a suspension of NaH (6.9 g, 172 mmol, 60% in mineral oil) in NMP (40 mL) at 0° C. was slowly added 2-trifluoromethyl-2-propanol (20 g, 156 mmol) and the mixture stirred at 0° C. until gas evolution ceased. Allyl bromide (13.3 mL, 156 mmol) was added and the reaction was stirred for 16 h while gradually warming to rt. The crude material was purified by distillation (atmospheric pressure, 190° C.) to provide the title compound as a clear oil.

### Intermediate 175

### (R,E)-2-Methyl-N-(2-((1,1,1-trifluoro-2-methylpropan-2-yl)oxy)ethylidene)propane-2-sulfinamide

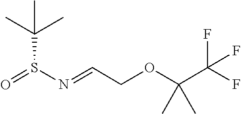 [SMILES: CC(C)(C)[S@@](=O)/N=C/COC(C)(C)C(F)(F)F]

A solution of 3-((1,1,1-trifluoro-2-methylpropan-2-yl)oxy)prop-1-ene (15.0 g, 89.2 mmol, Intermediate 174) in CH₂Cl₂ (300 mL) was cooled to −78° C. then treated with ozone for 20 min. The reaction headspace was purged with N2 then dimethyl sulfide (7.3 mL, 98. mmol) was added and the mixture allowed to warm to rt. After stirring for 4 h, (R)-2-methylpropane-2-sulfinamide (10.8 g, 89.2 mmol) and copper sulfate (43.6 g, 268 mmol) were added and then stirring was continued for 16 h. The mixture was filtered through Celite®, condensed, and purified by silica gel chromatography (0-60% EtOAc/hexanes) to provide the title compound.

### Intermediate 176

### 3,3-Difluoro-2-methylbutan-2-ol

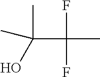

To a 0° C. solution of ethyl 2,2-difluoropropanoate (5.00 g, 36.2 mmol) in Et₂O (72 mL) was added methylmagnesium bromide (25 mL, 76 mmol, 3.0 M in Et₂O). The reaction was stirred for 3 h at 0° C. then quenched with saturated aqueous ammonium chloride. The aqueous layer was extracted with Et₂O (70 mL) then the combined organic layers were washed with brine, dried over anhydrous MgSO₄, filtered, and concentrated to dryness to provide the title compound which was used without further purification.

### Intermediate 177

### 2-(Allyloxy)-3,3-difluoro-2-methylbutane

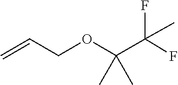

To a suspension of NaH (0.950 g, 23.8 mmol, 60% in mineral oil) in NMP (10 mL) at 0° C. was slowly added 3,3-difluoro-2-methylbutan-2-ol (4.47 g, 19.8 mmol, Intermediate 176) and the mixture stirred at 0° C. until gas evolution ceased. Allyl bromide (1.7 mL, 20 mmol) was added and the reaction was stirred for 16 h while gradually warming to rt. The reaction was poured into 5% aqueous lithium chloride (50 mL) and extracted with Et₂O (2×50 mL). The organic layers were combined, washed with 5% aqueous lithium chloride, dried over anhydrous Na₂SO₄, filtered, and condensed. The crude material was purified by silica gel chromatography (100% hexanes) to provide the title compound.

### Intermediate 178

### (R,E)-N-(2-((3,3-Difluoro-2-methylbutan-2-yl)oxy)ethylidene)-2-methylpropane-2-sulfinamide

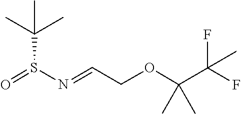 [SMILES: CC(C)(C)[S@@](=O)/N=C/COC(C)(C)C(C)(F)F]

To a solution of 2-(allyloxy)-3,3-difluoro-2-methylbutane (800 mg, 4.87 mmol, Intermediate 177) in THF (10 mL) and H₂O (1 mL) was added potassium osmate dihydrate (36 mg, 0.097 mmol). The reaction was stirred at rt for 15 min then a suspension of sodium periodate (2.08 g, 9.74 mmol) in H₂O (9 mL) was added. After 1 h at rt, the mixture was diluted with H₂O (15 mL) and extracted with DCM (2×25 mL). The combined organic layers were dried over anhydrous MgSO₄ and filtered. To this solution was added (R)-2-methylpropane-2-sulfinamide (0.492 g, 4.06 mmol), copper sulfate (1.94 g, 12.2 mmol), and PPTS (100 mg, 0.406 mmol). The resulting suspension was stirred at rt for 16 h then diluted with hexanes (50 mL), filtered through Celite®, and concentrated. Purification by silica gel chromatography (50% DCM/hexanes) provided the title compound.

### Intermediate 179

### 2-((1,1,1-Trifluoropropan-2-yl)oxy)acetic acid

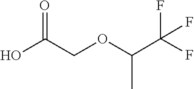

To a stirred suspension of sodium hydride (4.4 g, 110 mmol) and potassium iodide (463 mg, 2.76 mmol) in THF (250 mL) at 0° C. was added 1,1,1-trifluoro-2-propanol (5.0 mL, 55 mmol). The mixture was stirred for 10 min at 0° C. then a solution of bromoacetic acid (10.2 g, 73.5 mmol) in THF (100 mL) was added and the reaction was heated to 60° C. for 20 h. The mixture was condensed and extracted with H₂O (2×100 mL). Then the aqueous layers were combined and washed with EtOAc (2×50 mL), acidified with aqueous HCl (55 mL, 110.5 mmol, 2 M), and extracted with EtOAc (3×100 mL). The organic layers were combined, dried over anhydrous MgSO₄, filtered, and concentrated to provide the title compound.

### Intermediate 180

### N-Methoxy-N-methyl-2-((1,1,1-trifluoropropan-2-yl)oxy)acetamide

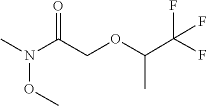

To a solution of 2-((1,1,1-trifluoropropan-2-yl)oxy)acetic acid (9.5 g, 55 mmol, Intermediate 179) in CH₂Cl₂ (110 mL) at 0° C. was added CDI (13.4 g, 82.5 mmol) in portions. The reaction was stirred at rt for 30 min then cooled to 0° C. Triethylamine (11.5 mL, 82.5 mmol) and N,O-dimethylhydroxylamine hydrochloride (8.21 g, 82.5 mmol) were added and the reaction was stirred for 72 h while gradually warming to rt. The solution was transferred to a separatory funnel, washed with 1 M aqueous HCl and saturated aqueous NaHCO₃, dried over anhydrous Na₂SO₄, filtered, and condensed to provide the title compound.

### Intermediate 181

### (R)-2-Methyl-N-((E)-2-((1,1,1-trifluoropropan-2-yl)oxy)ethylidene)propane-2-sulfinamide

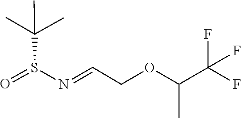 [SMILES: CC(OC/C=N/[S@](=O)C(C)(C)C)C(F)(F)F]

To a −78° C. solution of N-methoxy-N-methyl-2-((1,1,1-trifluoropropan-2-yl)oxy)acetamide (1.85 g, 8.60 mmol, Intermediate 180) in DCM (22 mL) was added LAH (4.3 mL, 8.6 mmol, 2 M in THF). The reaction was stirred for 1 h at −78° C. then quenched with acetone (1.9 mL), slowly warmed to rt, then poured into 30% aqueous Rochelle salt. The aqueous mixture was extracted with DCM. Then, the combined organic layers were washed with 30% aqueous Rochelle salt, 1 M aqueous HCl, and water, dried over anhydrous MgSO₄, and filtered through Celite®. To this solution was added (R)-2-methylpropane-2-sulfinamide (695 mg, 5.73 mmol), copper sulfate (2.75 g, 17.2 mmol), and PPTS (144 mg, 0.573 mmol). The resulting suspension was stirred at rt for 16 h then diluted with hexanes (50 mL), filtered through Celite®, and condensed. Purification by silica gel chromatography (50% DCM/hexanes) provided the title compound.

### Intermediate 182

### (R)-1-(1-((2-(Trimethylsilyl)ethoxy)methyl)-1H-benzo[d]imidazol-6-yl)ethan-1-amine and (R)-1-(1-((2-(trimethylsilyl)ethoxy)methyl)-1H-benzo[d]imidazol-5-yl)ethan-1-amine

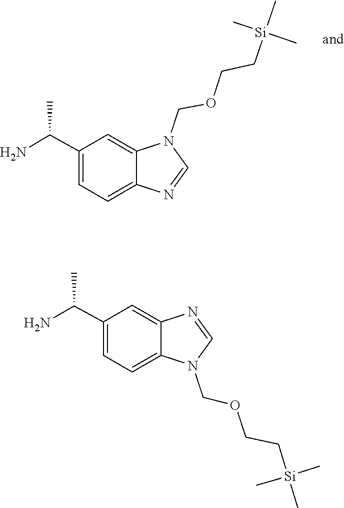 [SMILES: C[C@@H](N)c1ccc2ncn(COCC[Si](C)(C)C)c2c1 ; C[C@@H](N)c1ccc2c(c1)ncn2COCC[Si](C)(C)C]

To a suspension of (S)-2-methyl-N—((R)-1-(1-((2-(trimethylsilyl)ethoxy)methyl)-1H-benzo[d]imidazol-6-yl)ethyl)propane-2-sulfinamide and (S)-2-methyl-N—((R)-1-(1-((2-(trimethylsilyl)ethoxy)methyl)-1H-benzo[d]imidazol-5-yl)ethyl)propane-2-sulfinamide (2.00 g, 5.06 mmol, Intermediate 239) in EtOAc (25 mL) was added HCl (5.1 mL, 20 mmol, 4 M in 1,4-dioxane). The reaction was stirred at rt for 1 h then diluted with H₂O (50 mL). The resulting solution was washed twice with hexanes and the washes were discarded. The mixture was brought to basic pH by the addition of NaOH (0.81 g) in a minimum amount of H₂O then extracted three times with EtOAc. The combined organic layers were washed with brine, dried over anhydrous Na₂SO₄, filtered, and condensed to provide a mixture of the title compounds.

### Intermediate 183

### (R)—N—((R*)-1-(6-((R)-1-Aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(5-((R)-1-aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

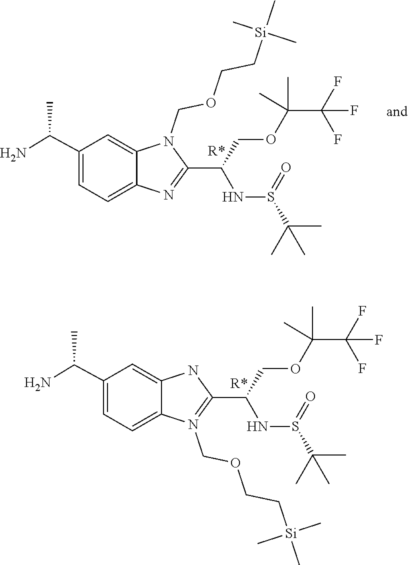

To a −78° C. solution of (R)-1-(1-((2-(trimethylsilyl)ethoxy)methyl)-1H-benzo[d]imidazol-6-yl)ethan-1-amine and (R)-1-(1-((2-(trimethylsilyl)ethoxy)methyl)-1H-benzo[d]imidazol-5-yl)ethan-1-amine (142 mg, 0.488 mmol, Intermediate 182) in THF (5 mL) was added n-BuLi (0.37 mL, 0.59 mmol, 1.6 M in hexanes). After stirring for 30 min at −78° C., (R,E)-2-methyl-N-(2-((1,1,1-trifluoro-2-methylpropan-2-yl)oxy)ethylidene)propane-2-sulfinamide (200 mg, 0.732 mmol, Intermediate 175) was added as a solution in THF (1 mL). The reaction was stirred for 30 min at −78° C. then quenched with EtOH (0.057 mL), diluted with EtOAc, and warmed to rt. The mixture was poured over brine and the aqueous layer extracted twice with EtOAc. Then, the combined organic layers were dried over anhydrous Na₂SO₄, filtered, and condensed to provide the mixture of title compounds.

### Intermediate 184

### N—((R)-1-(2-((R*)-1-(((R)-tert-Butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-6-yl)ethyl)-2-(3,3-difluorocyclobutyl)acetamide and N—((R)-1-(2-((R*)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)ethyl)-2-(3,3-difluorocyclobutyl)acetamide

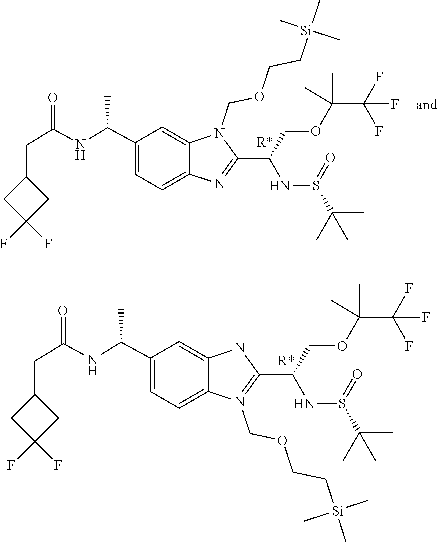

A solution of (R)—N—((R*)-1-(6-((R)-1-aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R*)-1-(5-((R)-1-aminoethyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (276 mg, 0.489 mmol, Intermediate 183), 2-(3,3-difluorocyclobutyl)acetic acid (88 mg, 0.59 mmol), DIPEA (0.15 mL, 0.88 mmol) and HOBt (79 mg, 0.59 mmol) in MeCN (6 mL) was heated to 45° C. and then EDCI (112 mg, 0.586 mmol) was added. The reaction was stirred for 20 min at 45° C. then quenched with H₂O and condensed. The residue was dissolved in EtOAc and washed with saturated aqueous ammonium chloride, saturated aqueous sodium bicarbonate, and brine. The organic layer was then dried over anhydrous Na₂SO₄, filtered, and condensed. Purification by silica gel chromatography (5-100% (10% MeOH/EtOAc)/hexanes) provided the mixture of title compounds.

### Intermediate 185

### N—((R)-1-(2-((R*)-1-Amino-2-((1,1,1-trifluoro-2-methylpropan-2-yl)oxy)ethyl)-1H-benzo[d]imidazol-6-yl)ethyl)-2-(3,3-difluorocyclobutyl)acetamide

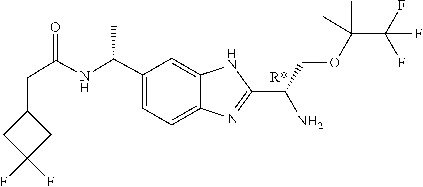 [SMILES: C[C@@H](NC(=O)CC1CC(F)(F)C1)c1ccc2nc([C@@H](N)COC(C)(C)C(F)(F)F)[nH]c2c1]

To a solution of N—((R)-1-(2-((R*)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-6-yl)ethyl)-2-(3,3-difluorocyclobutyl)acetamide and N—((R)-1-(2-((R*)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)ethyl)-2-(3,3-difluorocyclobutyl)acetamide (340 mg, 0.488 mmol, Intermediate 184) in 1,4-dioxane (4 mL) and MeOH (1 mL) was added HCl (1.7 mL, 6.7 mmol, 4 M in 1,4-dioxane) and the reaction was heated to 55° C. After 4 h, the mixture was cooled to rt, diluted with H₂O (15 mL) and washed with hexanes (2×10 mL). The aqueous layer was basified to pH>10 by the addition of 3 M aqueous sodium hydroxide then extracted with EtOAc (3×10 mL). The combined EtOAc extracts were dried over anhydrous Na₂SO₄, filtered, and condensed to provide the title compound that was used without further purification.

### Intermediate 186

### (R)-Cyclopropyl(1-((2-(trimethylsilyl)ethoxy)methyl)-1H-benzo[d]imidazol-6-yl)methanamine

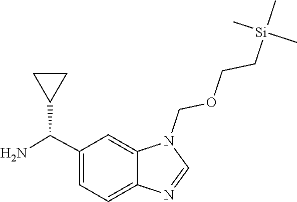 [SMILES: C[Si](C)(C)CCOCn1cnc2ccc([C@H](N)C3CC3)cc21]

To a solution of (S)—N—((R)-cyclopropyl(1-((2-(trimethylsilyl)ethoxy)methyl)-1H-benzo[d]imidazol-6-yl)methyl)-2-methylpropane-2-sulfinamide (1.1 g, 2.5 mmol, Intermediate 244) in EtOAc (12 mL) was added HCl (2.5 mL, 10 mmol, 4 M in 1,4-dioxane). The mixture was stirred for 2 h at rt, diluted with Et₂O, and filtered. The resulting solid was washed with Et₂O and air dried. The solid was then partitioned between EtOAc and 1 M aqueous NaOH. The aqueous layer was extracted three times with EtOAc, and then the combined organic layers were dried over anhydrous Na₂SO₄, filtered, and condensed to provide the title compound.

### Intermediate 187

### (R)—N-((1R*)-1-(6-((R)-Amino(cyclopropyl)methyl)-1-((2-(trimethylsilyl)ethoxy)methyl)-1H-benzo[d]imidazol-2-yl)-2-((1,1,1-trifluoropropan-2-yl)oxy)ethyl)-2-methylpropane-2-sulfinamide

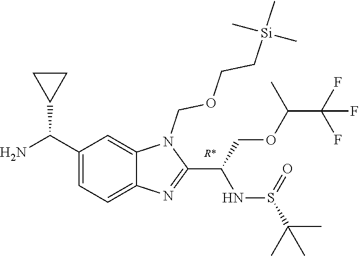

To a −78° C. solution of (R)-cyclopropyl(1-((2-(trimethylsilyl)ethoxy)methyl)-1H-benzo[d]imidazol-6-yl)methanamine (184 mg, 0.578 mmol, Intermediate 186) in THF (6 mL) was added n-BuLi (0.43 mL, 0.69 mmol, 1.6 M in hexanes). After stirring for 30 min at −78° C., (R)-2-methyl-N-((E)-2-((1,1,1-trifluoropropan-2-yl)oxy)ethylidene)propane-2-sulfinamide (195 mg, 0.752 mmol, Intermediate 181) was added as a solution in THF (1 mL). The reaction was stirred at −78° C. for 30 min then quenched with EtOH, diluted with EtOAc, and warmed to rt. The mixture was poured over brine and the aqueous layer extracted twice with EtOAc. Then, the combined organic layers were dried over anhydrous Na₂SO₄, filtered, and condensed to provide the title compound.

### Intermediate 188

### N-((1R)-(2-((1R*)-1-(((R)-tert-Butylsulfinyl)amino)-2-((1,1,1-trifluoropropan-2-yl)oxy)ethyl)-1-((2-(trimethylsilyl)ethoxy)methyl)-1H-benzo[d]imidazol-6-yl)(cyclopropyl)methyl)-2-(3,3-difluorocyclobutyl)acetamide

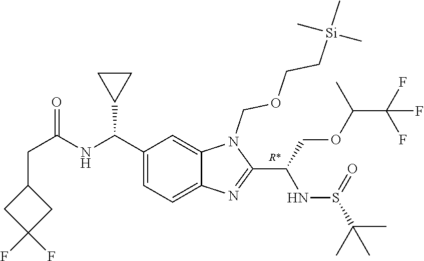

A solution of (R)—N-((1R*)-1-(6-((R)-amino(cyclopropyl)methyl)-1-((2-(trimethylsilyl)ethoxy)methyl)-1H-benzo[d]imidazol-2-yl)-2-((1,1,1-trifluoropropan-2-yl)oxy)ethyl)-2-methylpropane-2-sulfinamide (333 mg, 0.578 mmol, Intermediate 187), 2-(3,3-difluorocyclobutyl)acetic acid (104 mg, 0.694 mmol), DIPEA (0.18 mL, 1.0 mmol) and HOBt (94 mg, 0.69 mmol) in MeCN (6 mL) was heated to 45° C. and then EDCI (133 mg, 0.694 mmol) was added. The reaction was stirred at 45° C. for 20 min then quenched with H₂O and condensed. The residue was dissolved in EtOAc and washed with saturated aqueous ammonium chloride, saturated aqueous sodium bicarbonate and brine. Then, the organic layer was dried over anhydrous Na₂SO₄, filtered and condensed. Purification by silica gel chromatography (5-100% (10% MeOH/EtOAc)/hexanes) provided the title compound.

### Intermediate 189

### N-((1R)-(2-((1R*)-1-Amino-2-((1,1,1-trifluoropropan-2-yl)oxy)ethyl)-1H-benzo[d]imidazol-6-yl)(cyclopropyl)methyl)-2-(3,3-difluorocyclobutyl)acetamide

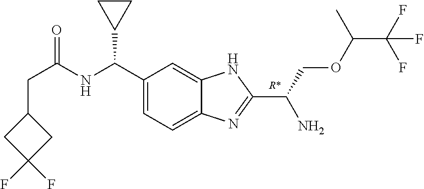 [SMILES: CC(OC[C@H](N)c1nc2ccc([C@H](NC(=O)CC3CC(F)(F)C3)C3CC3)cc2[nH]1)C(F)(F)F]

To a solution of N-((1R)-(2-((1R*)-1-(((R)-tert-butylsulfinyl)amino)-2-((1,1,1-trifluoropropan-2-yl)oxy)ethyl)-1-((2-(trimethylsilyl)ethoxy)methyl)-1H-benzo[d]imidazol-6-yl)(cyclopropyl)methyl)-2-(3,3-difluorocyclobutyl)acetamide (114 mg, 0.161 mmol, Intermediate 188) in 1,4-dioxane (1.3 mL) and MeOH (0.3 mL) was added HCl (0.6 mL, 2.2 mmol, 4 M in 1,4-dioxane) and the reaction heated to 50° C. After 6 h, the mixture was cooled to rt, diluted with H₂O (5 mL) and washed with hexanes (2×5 mL). The aqueous layer was basified to pH>10 by addition of 3 M aqueous sodium hydroxide then extracted with EtOAc (3×5 mL). The combined EtOAc extracts were dried over anhydrous Na₂SO₄, filtered, and condensed to provide the title compound.

### Intermediate 190

### (R)—N—((R*)-1-(5-((R)-Cyclopropyl(1,3-dioxoisoindolin-2-yl)methyl)-1-((2-(trimethylsilyl)ethoxy)methyl)-1H-benzo[d]imidazol-2-yl)-2-((3,3-difluoro-2-methylbutan-2-yl)oxy)ethyl)-2-methylpropane-2-sulfinamide

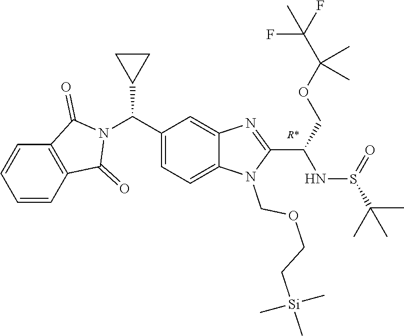

To a −78° C. solution of (R)-2-(cyclopropyl(1-((2-(trimethylsilyl)ethoxy)methyl)-1H-benzo[d]imidazol-5-yl)methyl)isoindoline-1,3-dione (892 mg, 1.99 mmol, Intermediate 262) and (R,E)-N-(2-((3,3-difluoro-2-methylbutan-2-yl)oxy)ethylidene)-2-methylpropane-2-sulfinamide (805 mg, 2.99 mmol, Intermediate 178) in THF (20 mL) was added LDA (3.2 mL, 3.8 mmol, 1.2 M in THF/hexanes). The reaction was stirred at −78° C. for 30 min then quenched with AcOH (0.23 mL), warmed to rt, and poured into a mixture saturated aqueous ammonium chloride (20 mL) and brine (50 mL). The mixture was extracted with EtOAc (2×100 mL), then the combined organic layers were washed with brine, dried over anhydrous Na₂SO₄, filtered, and condensed. Purification by silica gel chromatography (0-100% EtOAc/DCM) provided the title compound.

### Intermediate 191

### (R)—N—((R*)-1-(5-((R)-Amino(cyclopropyl)methyl)-1-((2-(trimethylsilyl)ethoxy)methyl)-1H-benzo[d]imidazol-2-yl)-2-((3,3-difluoro-2-methylbutan-2-yl)oxy)ethyl)-2-methylpropane-2-sulfinamide

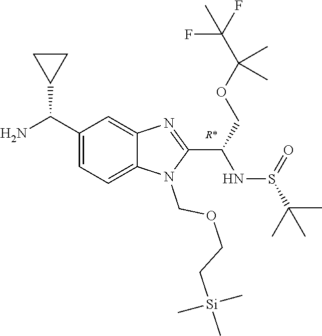

To a solution of (R)—N—((R*)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-((3,3-difluoro-2-methylbutan-2-yl)oxy)ethyl)-2-methylpropane-2-sulfinamide (400 mg, 0.558 mmol, Intermediate 190) in EtOH (3 mL) was added hydrazine monohydrate (0.39 mL, 5.2 mmol). The reaction was stirred for 4 h at rt then condensed. The residue was dissolved in EtOAc, the precipitate removed by filtration, and then the filtrate was condensed to provide the title compound.

### Intermediate 192

### N—((R)-(2-((R*)-1-(((R)-tert-Butylsulfinyl)amino)-2-((3,3-difluoro-2-methylbutan-2-yl)oxy)ethyl)-1-((2-(trimethylsilyl)ethoxy)methyl)-1H-benzo[d]imidazol-5-yl)(cyclopropyl)methyl)-2-(3,3-difluorocyclobutyl)acetamide

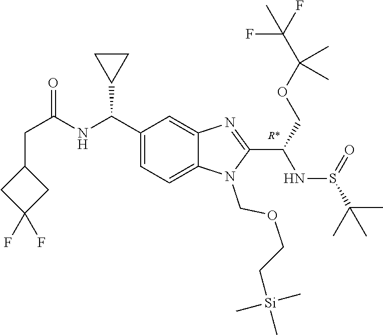

A solution of (R)—N—((R*)-1-(5-((R)-amino(cyclopropyl)methyl)-1-((2-(trimethylsilyl)ethoxy)methyl)-1H-benzo[d]imidazol-2-yl)-2-((3,3-difluoro-2-methylbutan-2-yl)oxy)ethyl)-2-methylpropane-2-sulfinamide (327 mg, 0.558 mmol, Intermediate 191), 2-(3,3-difluorocyclobutyl)acetic acid (126 mg, 0.837 mmol), DIPEA (0.24 mL, 1.4 mmol), and HOBt (113 mg, 0.837 mmol) in MeCN (6 mL) was heated to 45° C. and then EDCI (160 mg, 0.837 mmol) was added. The reaction was stirred at 45° C. for 20 min, then quenched with H₂O and condensed. The residue was dissolved in EtOAc and washed with saturated aqueous ammonium chloride, saturated aqueous sodium bicarbonate, and brine. Then, the organic layer was dried over anhydrous Na₂SO₄, filtered, and condensed. Purification by silica gel chromatography (10-80% (10% MeOH/EtOAc)/hexanes) provided the title compound.

### Intermediate 193

### N—((R)-(2-((R*)-1-Amino-2-((3,3-difluoro-2-methylbutan-2-yl)oxy)ethyl)-1H-benzo[d]imidazol-5-yl)(cyclopropyl)methyl)-2-(3,3-difluorocyclobutyl)acetamide

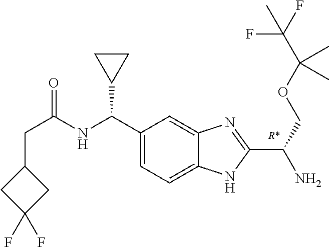 [SMILES: CC(F)(F)C(C)(C)OC[C@H](N)c1nc2cc([C@H](NC(=O)CC3CC(F)(F)C3)C3CC3)ccc2[nH]1]

To a solution of N—((R)-(2-((R*)-1-(((R)-tert-butylsulfinyl)amino)-2-((3,3-difluoro-2-methylbutan-2-yl)oxy)ethyl)-1-((2-(trimethylsilyl)ethoxy)methyl)-1H-benzo[d]imidazol-5-yl)(cyclopropyl)methyl)-2-(3,3-difluorocyclobutyl)acetamide (310 mg, 0.431 mmol, Intermediate 192) in 1,4-dioxane (3.6 mL) and MeOH (0.9 mL) was added HCl (1.1 mL, 4.3 mmol, 4 M in 1,4-dioxane) and the reaction heated to 60° C. After 4 h, the mixture was cooled to rt, diluted with H₂O (15 mL) and washed with 3:2 EtOAc:hexanes (2×5 mL). The aqueous layer was made basic to pH>10 by the addition of sodium hydroxide (200 mg) in H₂O (5 mL) then extracted with EtOAc (3×15 mL). The combined EtOAc extracts were dried over anhydrous Na₂SO₄, filtered, and condensed to provide the title compound.

### Intermediate 194

### (R)—N—((R)-1-(5-((R)-Cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

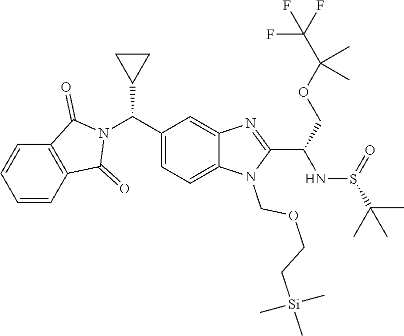

To a −78° C. solution of (R)-2-(cyclopropyl(1-((2-(trimethylsilyl)ethoxy)methyl)-1H-benzo[d]imidazol-5-yl)methyl)isoindoline-1,3-dione (3190 mg, 6.97 mmol, Intermediate 262) and (R,E)-2-methyl-N-(2-((1,1,1-trifluoro-2-methylpropan-2-yl)oxy)ethylidene)propane-2-sulfinamide (2860 mg, 10.5 mmol, Intermediate 175) in THF (70 mL) was added LDA (11 mL, 13 mmol, 1.2 M in THF/hexanes). The reaction was stirred at −78° C. for 30 min then quenched with AcOH (0.8 mL), warmed to rt, and poured into a mixture saturated aqueous ammonium chloride (20 mL) and brine (50 mL). The mixture was extracted with EtOAc (2×100 mL). Then the combined organic layers were washed with brine, dried over anhydrous Na₂SO₄, filtered, and condensed. Purification by silica gel chromatography (0-100% EtOAc/DCM) provided the title compound.

### Intermediate 195

### (R)—N—((R)-1-(5-((R)-Amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

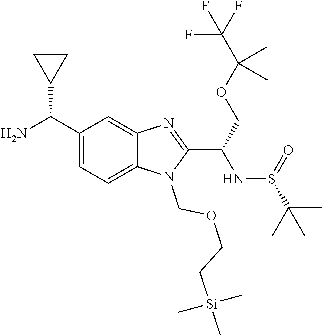

To a solution of (R)—N—((R)-1-(5-((R)-cyclopropyl(1,3-dioxoisoindolin-2-yl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (5640 mg, 7.82 mmol, Intermediate 194) in EtOH (78 mL) was added hydrazine monohydrate (3.5 mL, 48 mmol). The reaction was stirred for 4 h at rt then condensed. The residue was dissolved in EtOAc, the precipitate removed by filtration, and then the filtrate was condensed to provide the title compound.

### Intermediate 196

### N—((R)-(2-((R)-1-(((R)-tert-Butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)-2-(3,3-difluorocyclobutyl)acetamide

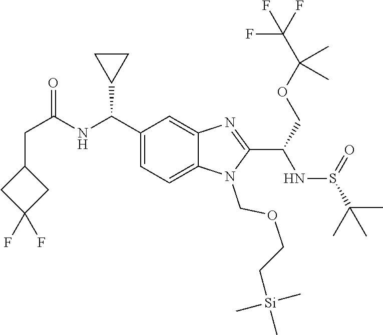

A solution of (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (6050 mg, 10.2 mmol, Intermediate 195), 2-(3,3-difluorocyclobutyl)acetic acid (2310 mg, 15.4 mmol), DIPEA (4.41 mL, 25.6 mmol), and HOBt (2080 mg, 15.4 mmol) in MeCN (50 mL) was heated to 45° C. and then EDCI (2940 mg, 15.4 mmol) was added. The reaction was stirred at 45° C. for 20 min then quenched with H₂O and condensed. The residue was dissolved in EtOAc and washed with saturated aqueous ammonium chloride, saturated aqueous sodium bicarbonate and brine. Then the organic layer was dried over anhydrous Na₂SO₄, filtered, and condensed. Purification by silica gel chromatography (10-80% (10% MeOH/EtOAc)/hexanes) provided the title compound.

### Intermediate 197

### N—((R)-(2-((R)-1-Amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide

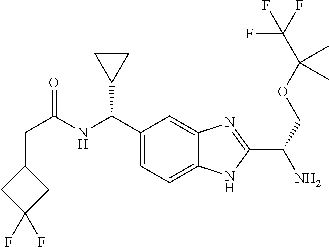 [SMILES: CC(C)(OC[C@H](N)c1nc2cc([C@H](NC(=O)CC3CC(F)(F)C3)C3CC3)ccc2[nH]1)C(F)(F)F]

To a solution of N—((R)-(2-((R)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)-2-(3,3-difluorocyclobutyl)acetamide (3960 mg, 5.47 mmol, Intermediate 196) in 1,4-dioxane (40 mL) and MeOH (10 mL) was added HCl (13.7 mL, 54.7 mmol, 4 M in 1,4-dioxane) and the reaction heated to 65° C. After 4 h, the mixture was cooled to rt, diluted with H₂O (150 mL) and washed with 4:1 EtOAc:hexanes (3×50 mL). The aqueous layer was basified to pH>10 by the addition of NaOH (2.5 g) in H₂O (20 mL) then extracted with EtOAc (3×100 mL). The combined EtOAc extracts were dried over anhydrous Na₂SO₄, filtered, and concentrated to provide the title compound.

### Intermediate 198

### 2-((1,1,1-Trifluoro-2-methylpropan-2-yl)oxy)acetic acid

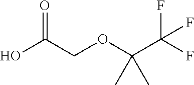

The title compound was prepared as described for the synthesis of Intermediate 179, using 2-trifluoromethyl-2-propanol in place of 1,1,1-trifluoro-2-propanol.

### Intermediate 199

### N-Methoxy-N-methyl-2-((1,1,1-trifluoro-2-methylpropan-2-yl)oxy)acetamide

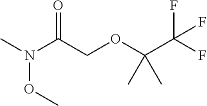

The title compound was prepared as described for the synthesis of Intermediate 180, using 2-((1,1,1-trifluoro-2-methylpropan-2-yl)oxy)acetic acid (Intermediate 198) in place of 2-((1,1,1-trifluoropropan-2-yl)oxy)acetic acid.

### Intermediate 200

### (S,E)-2-methyl-N-(2-((1,1,1-trifluoro-2-methylpropan-2-yl)oxy)ethylidene)propane-2-sulfinamide

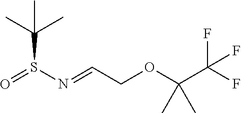 [SMILES: CC(C)(C)[S@](=O)/N=C/COC(C)(C)C(F)(F)F]

The title compound was prepared as described for the synthesis of Intermediate 181, using N-methoxy-N-methyl-2-((1,1,1-trifluoro-2-methylpropan-2-yl)oxy)acetamide (Intermediate 199) instead of N-methoxy-N-methyl-2-((1,1,1-trifluoropropan-2-yl)oxy)acetamide and (S)-2-methylpropane-2-sulfinamide in place of (R)-2-methylpropane-2-sulfinamide.

### Intermediate 201

### (S)—N—((S*)-1-(6-((R)-Amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

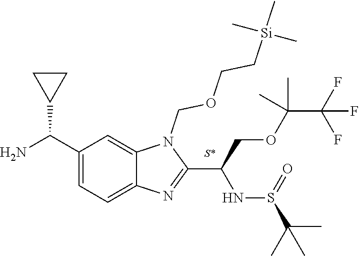

To a −78° C. solution of (R)-cyclopropyl(1-((2-(trimethylsilyl)ethoxy)methyl)-1H-benzo[d]imidazol-6-yl)methanamine (110 mg, 0.346 mmol, Intermediate 186) in THF (4 mL) was added n-butyllithium (0.28 mL, 0.45 mmol, 1.6 M in hexanes). After stirring for 30 min at −78° C., (S,E)-2-methyl-N-(2-((1,1,1-trifluoro-2-methylpropan-2-yl)oxy)ethylidene)propane-2-sulfinamide (140 mg, 0.52 mmol, Intermediate 200) was added as a solution in THF (1 mL). The reaction was stirred at −78° C. for 30 min then quenched with EtOH, diluted with EtOAc, and warmed to rt. The mixture was poured over brine and the aqueous layer extracted twice with EtOAc. Then, the combined organic layers were dried over anhydrous Na₂SO₄, filtered, and condensed to provide the title compound.

### Intermediate 202

### N—((R)-(2-((S*)-1-(((S)-tert-Butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-6-yl)(cyclopropyl)methyl)-2-(3,3-difluorocyclobutyl)acetamide

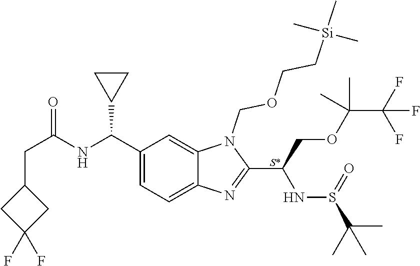 [SMILES: CC(C)(C)[S@](=O)N[C@H](COC(C)(C)C(F)(F)F)c1nc2ccc([C@H](NC(=O)CC3CC(F)(F)C3)C3CC3)cc2n1COCC[Si](C)(C)C]

A solution of (S)—N—((S*)-1-(6-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (204 mg, 0.346 mmol, Intermediate 201), 2-(3,3-difluorocyclobutyl)acetic acid (168 mg, 1.12 mmol), DIPEA (0.28 mL, 1.6 mmol) and HOBt (151 mg, 1.12 mmol) in MeCN (6 mL) was heated to 45° C. and then EDCI (215 mg, 1.12 mmol) was added. The reaction was stirred at 45° C. for 90 min then quenched with H₂O and condensed. The residue was dissolved in EtOAc and washed with saturated aqueous ammonium chloride, saturated aqueous sodium bicarbonate and brine. Then, the organic layer was dried over anhydrous Na₂SO₄, filtered, and condensed. Purification by silica gel chromatography (5-100% (10% MeOH/EtOAc)/hexanes) provided the title compound.

### Intermediate 203

### N—((R)-(2-((S*)-1-Amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide

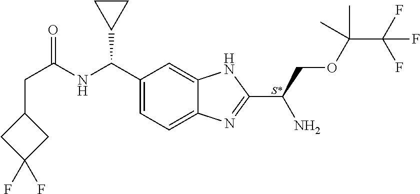 [SMILES: CC(C)(OC[C@@H](N)c1nc2ccc([C@H](NC(=O)CC3CC(F)(F)C3)C3CC3)cc2[nH]1)C(F)(F)F]

To a solution of N—((R)-(2-((S*)-1-(((S)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-6-yl)(cyclopropyl)methyl)-2-(3,3-difluorocyclobutyl)acetamide (100 mg, 0.138 mmol, Intermediate 202) in 1,4-dioxane (0.5 mL) and MeOH (0.1 mL) was added HCl (0.5 mL, 1.9 mmol, 4 M in 1,4-dioxane) and the reaction was heated to 50° C. After 4 h, the mixture was cooled to rt, diluted with H₂O and washed twice with hexanes. The aqueous layer was basified to pH>10 by addition of 3 M aqueous sodium hydroxide then extracted three times with EtOAc. The combined EtOAc extracts were dried over anhydrous Na₂SO₄, filtered, and condensed to provide the title compound.

### Intermediate 204

### N—((R)-(2-((R)-1-(((R)-tert-Butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)-2-(3-fluorobicyclo[1.1.1]pentan-1-yl)acetamide

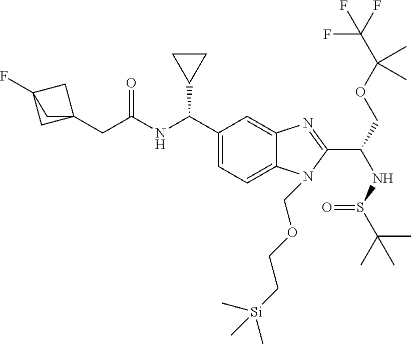

A solution of 2-(3-fluorobicyclo[1.1.1]pentan-1-yl)acetic acid (110 mg, 0.76 mmol), DIPEA (0.28 mL, 1.6 mmol) and EDCI (230 mg, 1.2 mmol) in DCM (6 mL) was stirred for 10 min at rt then (R)—N—((R)-1-(5-((R)-amino(cyclopropyl)methyl)-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (240 mg, 0.40 mmol, Intermediate 195) was added. The reaction was stirred at rt overnight then diluted with H₂O (30 mL) and DCM (30 mL). The mixture was separated, and the aqueous phase was extracted with DCM (3×30 mL). The combined organic phases were washed with water (30 mL) and brine (50 mL), dried over anhydrous Na₂SO₄, filtered, and concentrated to dryness to afford the crude product which was purified by silica gel chromatography (0-2% MeOH/DCM) to afford the title compound.

### Intermediate 205

### N—((R)-(2-((R)-1-Amino-2-((1,1,1-trifluoro-2-methylpropan-2-yl)oxy)ethyl)-1H-benzo[d]imidazol-5-yl)(cyclopropyl)methyl)-2-(3-fluorobicyclo[1.1.1]pentan-1-yl)acetamide hydrochloride

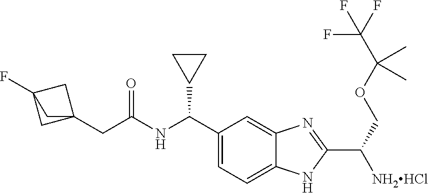

To a solution of N—((R)-(2-((R)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)-2-(3-fluorobicyclo[1.1.1]pentan-1-yl)acetamide (250 mg, 0.349 mmol, Intermediate 204) in 1,4-dioxane (4 mL) was added HCl (3.96 mL, 15.8 mmol, 4 M in 1,4-dioxane). The reaction was stirred at 55° C. for 2 h then condensed to afford the title compound.

### Intermediate 206

### (R,E)-2-Methyl-N-(spiro[2.5]octan-6-ylmethylene)propane-2-sulfinamide

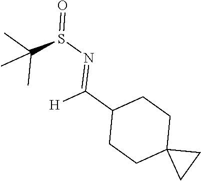 [SMILES: CC(C)(C)[S@@](=O)/N=C/C1CCC2(CC1)CC2]

The title compound was prepared as described for the synthesis of Intermediate 234, using spiro[2.5]octane-6-carbaldehyde in place of 4,4-difluorocyclohexane-1-carbaldehyde. The product was purified by silica gel chromatography (0-20% EtOAc/petroleum ether) to afford the title compound as a colorless oil.

### Intermediate 207

### Ethyl 4,4-difluorotetrahydro-2H-pyran-2-carboxylate

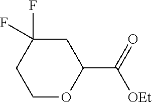 [SMILES: CCOC(=O)C1CC(F)(F)CCO1]

DAST (7.4 mL, 60 mmol) was added to a solution of ethyl 4-oxotetrahydro-2H-pyran-2-carboxylate (5.0 g, 29 mmol) in anhydrous DCM (60 mL) at 0° C. After complete addition of DAST, the reaction was allowed to warm to rt and stir for 2 h. Upon complete consumption of starting material, the reaction mixture was poured into saturated aqueous NaHCO₃ solution (100 mL), and the aqueous phase was extracted with DCM (2×150 mL). The combined organic layers were washed with brine (100 mL), dried over anhydrous Na₂SO₄, filtered, and concentrated under reduced pressure. The residue was purified by silica gel chromatography (0-5% EtOAc/petroleum ether) to afford the title compound as a colorless liquid.

### Intermediate 208

### 4,4-Difluorotetrahydro-2H-pyran-2-carbaldehyde

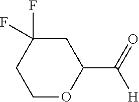

The title compound was prepared as described for the synthesis of Intermediate WX-17, using ethyl 4,4-difluorotetrahydro-2H-pyran-2-carboxylate (Intermediate 207) in place of (1R,3s,5S)-6,6-difluorobicyclo[3.1.0]hexane-3-carbaldehyde and (1R,3r,5S)-6,6-difluorobicyclo[3.1.0]hexane-3-carbaldehyde. The product was used in the next step without further purification.

### Intermediate 209

### (R)—N-((E)-(4,4-Difluorotetrahydro-2H-pyran-2-yl)methylene)-2-methylpropane-2-sulfinamide

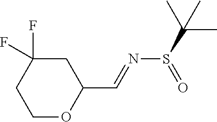 [SMILES: CC(C)(C)[S@@](=O)/N=C/C1CC(F)(F)CCO1]

The title compound was prepared as described for the synthesis of Intermediate 234, using 4,4-difluorotetrahydro-2H-pyran-2-carbaldehyde (Intermediate 208) in place of 4,4-difluorocyclohexane-1-carbaldehyde. The product was purified by silica gel chromatography (0-10% EtOAc/petroleum ether) to afford the title compound as a pale-yellow oil.

### Intermediate 210

### (R)—N-((E)-(2,2-Dimethyltetrahydro-2H-pyran-4-yl)methylene)-2-methylpropane-2-sulfinamide

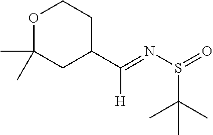 [SMILES: CC1(C)CC(/C=N/S(=O)C(C)(C)C)CCO1]

The title compound was prepared as described for the synthesis of Intermediate 234, using 2,2-dimethyltetrahydro-2H-pyran-4-carbaldehyde in place of 4,4-difluorocyclohexane-1-carbaldehyde. The product was purified by silica gel chromatography (0-30% EtOAc/petroleum ether) to afford the title compound as a pale-yellow oil.

### Intermediate 211

### N—((R)-(2-((S)—(((R)-tert-Butylsulfinyl)amino)(spiro[2.5]octan-6-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide

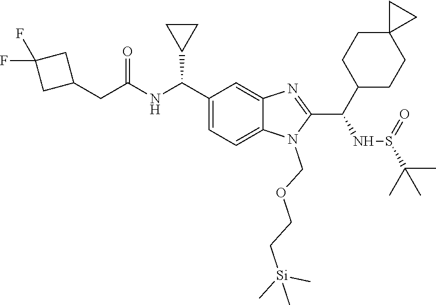 [SMILES: CC(C)(C)[S@@](=O)N[C@H](c1nc2cc([C@H](NC(=O)CC3CC(F)(F)C3)C3CC3)ccc2n1COCC[Si](C)(C)C)C1CCC2(CC1)CC2]

The title compound was prepared as described for the synthesis of Intermediate 344, using (R,E)-2-methyl-N-(spiro[2.5]octan-6-ylmethylene)propane-2-sulfinamide (Intermediate 206) in place of (R,E)-N-(4,4-difluoro-3,3-dimethylbutylidene)-2-methylpropane-2-sulfinamide and (R)—N-(cyclopropyl(1-((2-(trimethylsilyl)ethoxy)methyl)-1H-benzo[d]imidazol-5-yl)methyl)-2-(3,3-difluorocyclobutyl)acetamide (Intermediate 356) in place of (R)-cyclopropyl(1-((2-(trimethylsilyl)ethoxy)methyl)-1H-benzo[d]imidazol-5-yl)methanamine. The product was used without further purification.

### Intermediate 212

### N—((R)-(2-((S)-Amino(spiro[2.5]octan-6-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide

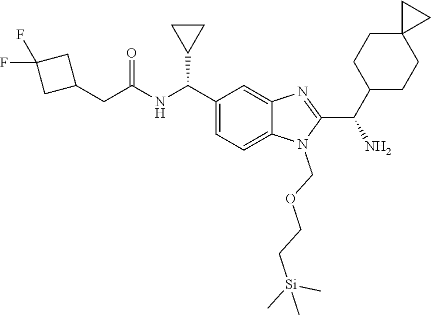 [SMILES: C[Si](C)(C)CCOCn1c([C@@H](N)C2CCC3(CC2)CC3)nc2cc([C@H](NC(=O)CC3CC(F)(F)C3)C3CC3)ccc21]

I₂ (7.3 mg, 0.03 mmol) was added to a solution of N—((R)-(2-((S)—(((R)-tert-butylsulfinyl)amino)(spiro[2.5]octan-6-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide (100 mg, 0.145 mmol, Intermediate 211) in THF (4 mL) and water (1 mL). The reaction mixture was then stirred at 50° C. for 16 h. After this time, the reaction was cooled to rt and was then diluted with water (20 mL) and concentrated under reduced pressure to remove the organic solvent. A saturated aqueous solution of sodium thiosulfate (20 mL) was added and the resultant aqueous solution was then washed with MTBE (2×20 mL). The aqueous layer was treated with saturated aqueous solution of NaHCO₃ (50 mL) and extracted with DCM (2×50 mL). The combined organic phases were washed with brine, dried over Na₂SO₄, filtered and concentrated. The crude product was purified by silica gel chromatography (5-10% MeOH/DCM) to afford the title compound as a colorless oil.

### Intermediate 213

### N—((S)-(5-((R)-Cyclopropyl(2-(3,3-difluorocyclobutyl)acetamido)methyl)-1-((2-(trimethylsilyl)ethoxy)methyl)-1H-benzo[d]imidazol-2-yl)(spiro[2.5]octan-6-yl)methyl)-4-methyl-1,2,5-oxadiazole-3-carboxamide

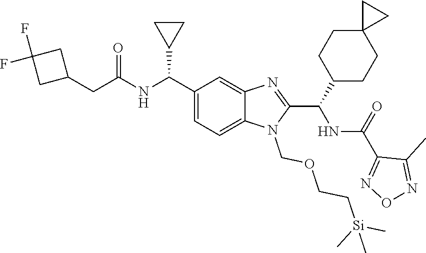 [SMILES: Cc1nonc1C(=O)N[C@H](c1nc2cc([C@H](NC(=O)CC3CC(F)(F)C3)C3CC3)ccc2n1COCC[Si](C)(C)C)C1CCC2(CC1)CC2]

The title compound was prepared as described for the synthesis of Example 233, using N—((R)-(2-((S)-amino(spiro[2.5]octan-6-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide (Intermediate 212) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The product was purified by silica gel chromatography (0-10% MeOH/DCM) to afford the title compound as a colorless oil.

### Intermediate 214

### (R)—N-(Cyclopropyl(1-((2-(trimethylsilyl)ethoxy)methyl)-1H-benzo[d]imidazol-6-yl)methyl)-2-(3,3-difluorocyclobutyl)acetamide

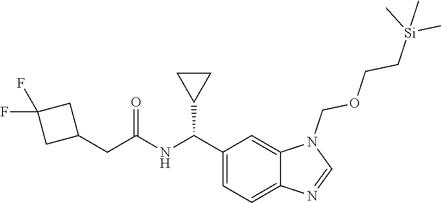 [SMILES: C[Si](C)(C)CCOCn1cnc2ccc([C@H](NC(=O)CC3CC(F)(F)C3)C3CC3)cc21]

The title compound was prepared as described for the synthesis of Intermediate 356, using (R)-cyclopropyl(1-((2-(trimethylsilyl)ethoxy)methyl)-1H-benzo[d]imidazol-5-yl)methanamine (Intermediate 186) in place of (R)-cyclopropyl(1-((2-(trimethylsilyl)ethoxy)methyl)-1H-benzo[d]imidazol-5-yl)methanamine. The product was purified by silica gel chromatography (0-5% MeOH/DCM) to afford the title compound as a yellow oil.

### Intermediate 215

### N—((R)-(2-((R)—(((R)-tert-Butylsulfinyl)amino)((S*)-4,4-difluorotetrahydro-2H-pyran-2-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide

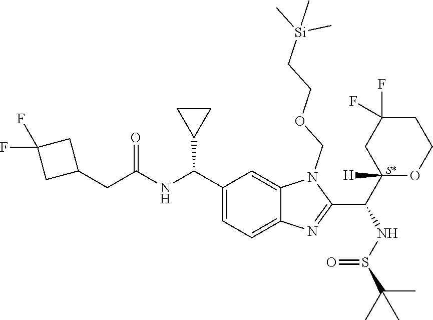 [SMILES: CC(C)(C)[S@@](=O)N[C@H](c1nc2ccc([C@H](NC(=O)CC3CC(F)(F)C3)C3CC3)cc2n1COCC[Si](C)(C)C)[C@@H]1CC(F)(F)CCO1]

The title compound was prepared as described for the synthesis of Intermediate 344, using (R)—N-((E)-(4,4-difluorotetrahydro-2H-pyran-2-yl)methylene)-2-methylpropane-2-sulfinamide (Intermediate 209) in place of (R,E)-N-(4,4-difluoro-3,3-dimethylbutylidene)-2-methylpropane-2-sulfinamide and (R)—N-(cyclopropyl(1-((2-(trimethylsilyl)ethoxy)methyl)-1H-benzo[d]imidazol-6-yl)methyl)-2-(3,3-difluorocyclobutyl)acetamide (Intermediate 214) in place of (R)-cyclopropyl(1-((2-(trimethylsilyl)ethoxy)methyl)-1H-benzo[d]imidazol-5-yl)methanamine. The product was purified by silica gel chromatography (0-10% MeOH/DCM) followed by SFC using a chiral stationary phase (DAICEL CHIRALCEL OD-H, 5 μm, 250×30 mm, 15% CO₂ in EtOH (0.1% NH₄OH)). The product containing fractions, first eluting peak, were diluted with water, frozen and lyophilized to afford the title compound as a white solid.

### Intermediate 216

### N—((R)-(2-((R)-Amino((S*)-4,4-difluorotetrahydro-2H-pyran-2-yl)methyl)-1H-benzo[d]imidazol-6-yl)(cyclopropyl)methyl)-2-(3,3-difluorocyclobutyl)acetamide

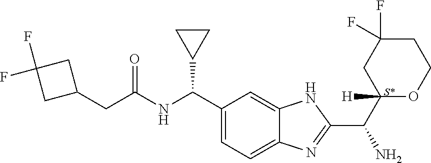 [SMILES: N[C@H](c1nc2ccc([C@H](NC(=O)CC3CC(F)(F)C3)C3CC3)cc2[nH]1)[C@@H]1CC(F)(F)CCO1]

The title compound was prepared as described for the synthesis of Intermediate 361, using N—((R)-(2-((R)—(((R)-tert-butylsulfinyl)amino)((S*)-4,4-difluorotetrahydro-2H-pyran-2-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 215) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide. The reaction mixture was concentrated to dryness and used without further purification.

### Intermediate 217

### (R)—N-((1S)-(5-((R)-Cyclopropyl(1,3-dioxoisoindolin-2-yl)methyl)-1-((2-(trimethylsilyl)ethoxy)methyl)-1H-benzo[d]imidazol-2-yl)(2,2-dimethyltetrahydro-2H-pyran-4-yl)methyl)-2-methylpropane-2-sulfinamide

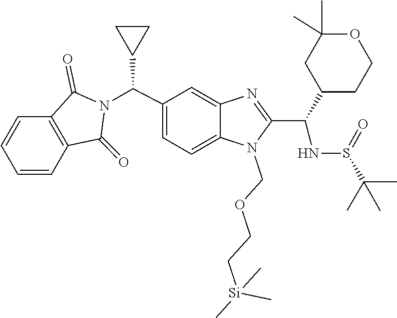 [SMILES: CC1(C)CC([C@H](N[S@](=O)C(C)(C)C)c2nc3cc([C@@H](C4CC4)N4C(=O)c5ccccc5C4=O)ccc3n2COCC[Si](C)(C)C)CCO1]

The title compound was prepared as described for the synthesis of Intermediate 264, using (R)—N-((E)-(2,2-dimethyltetrahydro-2H-pyran-4-yl)methylene)-2-methylpropane-2-sulfinamide (Intermediate 210) in place of (R,Z)-2-methyl-N-(4,4,4-trifluoro-3,3-dimethylbutylidene)propane-2-sulfinamide. The product was purified by silica gel chromatography (0-100% EtOAc/petroleum ether) to afford the title compound as a light yellow oil.

### Intermediate 218

### (R)—N-((1S)-(5-((R)-Amino(cyclopropyl)methyl)-1-((2-(trimethylsilyl)ethoxy)methyl)-1H-benzo[d]imidazol-2-yl)(2,2-dimethyltetrahydro-2H-pyran-4-yl)methyl)-2-methylpropane-2-sulfinamide

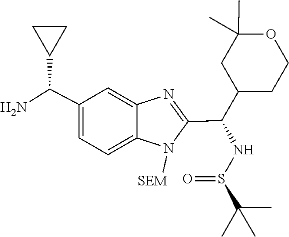 [SMILES: CC1(C)CC([C@H](N[S@](=O)C(C)(C)C)c2nc3cc([C@H](N)C4CC4)ccc3n2COCC[Si](C)(C)C)CCO1]

The title compound was prepared as described for the synthesis of Intermediate 265, using (R)—N-((1S)-(5-((R)-cyclopropyl(1,3-dioxoisoindolin-2-yl)methyl)-1-((2-(trimethylsilyl)ethoxy)methyl)-1H-benzo[d]imidazol-2-yl)(2,2-dimethyltetrahydro-2H-pyran-4-yl)methyl)-2-methylpropane-2-sulfinamide (Intermediate 217) in place of (R)—N—((S)-1-(5-((R)-cyclopropyl(1,3-dioxoisoindolin-2-yl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide. The product was used crude without further purification.

### Intermediate 219

### N-((1R)-(2-((1S)—(((R)-tert-Butylsulfinyl)amino)(2,2-dimethyltetrahydro-2H-pyran-4-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide

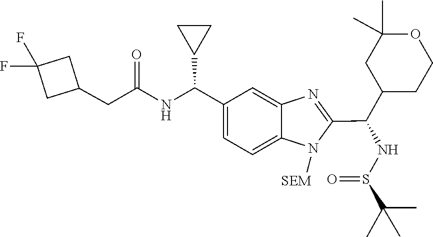 [SMILES: CC1(C)CC([C@H](N[S@](=O)C(C)(C)C)c2nc3cc([C@H](NC(=O)CC4CC(F)(F)C4)C4CC4)ccc3n2COCC[Si](C)(C)C)CCO1]

The title compound was prepared as described for the synthesis of Intermediate 360, using (R)—N-((1S)-(5-((R)-amino(cyclopropyl)methyl)-1-((2-(trimethylsilyl)ethoxy)methyl)-1H-benzo[d]imidazol-2-yl)(2,2-dimethyltetrahydro-2H-pyran-4-yl)methyl)-2-methylpropane-2-sulfinamide (Intermediate 218) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide. The product was purified by silica gel chromatography (0-100% EtOAc/petroleum ether) to afford the title compound as a light-yellow oil.

### Intermediate 220

### N-((1R)-(2-((1S)-Amino(2,2-dimethyltetrahydro-2H-pyran-4-yl)methyl)-1H-benzo[d]imidazol-6-yl)(cyclopropyl)methyl)-2-(3,3-difluorocyclobutyl)acetamide

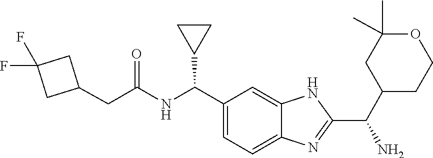 [SMILES: CC1(C)CC([C@H](N)c2nc3ccc([C@H](NC(=O)CC4CC(F)(F)C4)C4CC4)cc3[nH]2)CCO1]

The title compound was prepared as described for the synthesis of Intermediate 361, using N-((1R)-(2-((1S)—(((R)-tert-butylsulfinyl)amino)(2,2-dimethyltetrahydro-2H-pyran-4-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide (Intermediate 219) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide. The product was used without further purification.

### Intermediate 221

### Ethyl 4-cyclopropyl-5-(pyrrolidin-1-yl)-4,5-dihydroisoxazole-3-carboxylate

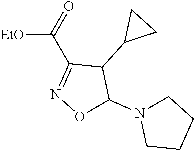 [SMILES: CCOC(=O)C1=NOC(N2CCCC2)C1C1CC1]

2-Cyclopropylacetaldehyde (1.5 g, 17.5 mmol) was added to a solution of pyrrolidine (1.6 mL, 19.2 mmol) and triethylamine (1.2 mL, 8.7 mmol) in DCM (44 mL) at 0° C. A solution of ethyl 2-chloro-2-(hydroxyamino)acetate (1.3 g, 8.7 mmol) in DCM (17 mL) was added in 5 portions over 5 min intervals. After 10 min, the ice bath was removed and the reaction was allowed to stir at rt for 1.5 h. The solution was then concentrated under reduced pressure and purified by silica gel chromatography (0-20% EtOAc/hexanes) to provide the title compound as a colorless oil.

### Intermediate 222

### Ethyl 4-cyclopropylisoxazole-3-carboxylate

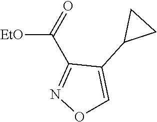 [SMILES: CCOC(=O)c1nocc1C1CC1]

mCPBA (1.6 g, 7.4 mmol) was added to a solution of ethyl 4-cyclopropyl-5-(pyrrolidin-1-yl)-4,5-dihydroisoxazole-3-carboxylate (1.2 g, 4.6 mmol, Intermediate 221) in DCM (18 mL) at rt. The reaction was stirred at rt for 2 h and was subsequently quenched with a saturated aqueous solution of NaHCO₃. The biphasic mixture was transferred to a separatory funnel and extracted with EtOAc (3×20 mL). The combined organic layers were washed with a saturated aqueous solution of NaHCO₃ and brine, dried over anhydrous MgSO₄, filtered, and concentrated under reduced pressure. Purification by silica gel chromatography (0-45% EtOAc/hexanes) yielded the title compound as a low melting colorless solid.

### Intermediate 223

### 4-Cyclopropylisoxazole-3-carboxylic acid

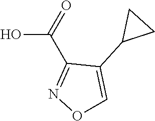 [SMILES: O=C(O)c1nocc1C1CC1]

Ethyl 4-cyclopropylisoxazole-3-carboxylate (674 mg, 3.7 mmol, Intermediate 222) was dissolved in THF (2.2 mL) and had a solution of LiOH (178 mg, 7.4 mmol) in DI water (3.7 mL, 7.4 mmol) added. The mixture was allowed to stir at rt until full consumption of starting material. At which time, the reaction was acidified with 1 N aqueous HCl (10 mL) and extracted with 20% IPA in CHCl₃ (3×20 mL). The combined organic layers were dried over anhydrous MgSO₄, filtered, and concentrated under reduced pressure. The crude product was purified by acidic preparative HPLC (Xbridge Prep C18, 5 μm, 50×100 mm, 5-95% MeCN (0.5% TFA) in water (0.5% TFA) and the product containing fractions were diluted with water, frozen, and lyophilized to dryness to afford the title compound as a white solid.

### Intermediate 224

### N-Phenyl-4-(2,2,2-trifluoroethoxy)-1,2,5-oxadiazole-3-carboxamide

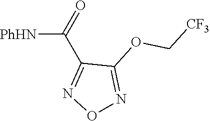

A round bottom flask was charged with NaH (60 wt % suspension in mineral oil, 1.0 g, 27 mmol) and THF (60 mL). 2,2,2-Trifluoroethanol (1.3 g, 13.4 mmol) was added dropwise to the NaH suspension at rt and stirred for 5 min at rt. Then, 4-chloro-N-phenyl-1,2,5-oxadiazole-3-carboxamide (2 g, 8.9 mmol) was added as a solid and the mixture was heated to 50° C. for 1.25 h. The reaction was then cooled to rt and quenched slowly with 1 N aqueous HCl. The aqueous layer was extracted three times with EtOAc and the combined organic layers were washed with brine, dried over anhydrous MgSO₄, filtered, and concentrated under reduced pressure. The crude material was purified by silica gel chromatography (0-17% EtOAc/hexanes) to provide the title compound as a pale-yellow solid.

### Intermediate 225

### tert-Butyl phenyl(4-(2,2,2-trifluoroethoxy)-1,2,5-oxadiazole-3-carbonyl)carbamate

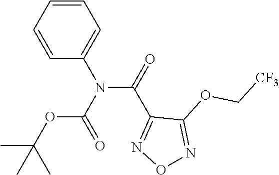 [SMILES: CC(C)(C)OC(=O)N(C(=O)c1nonc1OCC(F)(F)F)c1ccccc1]

Di-tert-butyl dicarbonate (475 mg, 2.2 mmol) and DMAP (21 mg, 0.17 mmol) were added sequentially to a solution of N-phenyl-4-(2,2,2-trifluoroethoxy)-1,2,5-oxadiazole-3-carboxamide (500 mg, 1.7 mmol, Intermediate 224) in DCM (8.7 mL) at rt and allowed to stir for 2 h. Upon complete consumption of starting material, the reaction was quenched with a saturated aqueous solution of NaHCO₃ (20 mL) and transferred to a separatory funnel. The biphasic mixture was extracted with EtOAc (3×20 mL) and the combined organic layers were washed with brine, dried over anhydrous MgSO₄, filtered, and concentrated under reduced pressure to afford crude title compound, which was used without further purification.

### Intermediate 226

### 4-(2,2,2-Trifluoroethoxy)-1,2,5-oxadiazole-3-carboxylic acid

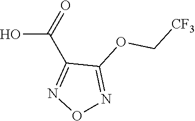 [SMILES: O=C(O)c1nonc1OCC(F)(F)F]

A solution of LiOH (230 mg, 9.6 mmol) in water (4.8 mL) was added dropwise to a solution of N-phenyl-4-(2,2,2-trifluoroethoxy)-1,2,5-oxadiazole-3-carboxamide (670 mg, 1.7 mmol, Intermediate 225) in THF (6.4 mL) and heated to 35° C. After 1.5 h, the reaction was acidified to pH 1 with 1 N aqueous HCl (10 mL) and extracted with EtOAc (3×5 mL). The combined organic layers were washed with a saturated aqueous solution of NaHCO₃ and the organic layer was discarded. The new aqueous layer was slowly reacidified to pH 1 with 6 N aqueous HCl and extracted with EtOAc (3×20 mL). The new combined organic layers were dried over anhydrous Na₂SO₄, filtered, and concentrated under reduced pressure to afford the title compound as a hygroscopic solid. The title compound was diluted in MeCN and water, frozen, and lyophilized to dryness to generate a white powder.

### Intermediate 227

### N-((1R)-(2-((1R)-1-((tert-Butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)-4,4-difluoro-3-methylbutanamide

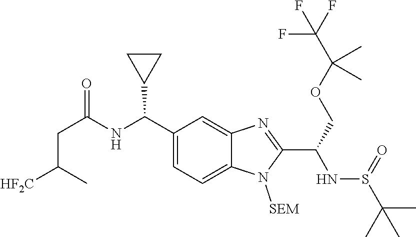

The title compound was prepared as described for the synthesis of Intermediate 196, using 4,4-difluoro-3-methylbutanoic acid in place of 2-(3,3-difluorocyclobutyl)acetic acid. The product was purified by silica gel chromatography (0-90% acetone/hexanes) to afford the title compound as a white foam.

### Intermediate 228

### N—((R)-(2-((R)-1-Amino-2-((1,1,1-trifluoro-2-methylpropan-2-yl)oxy)ethyl)-1H-benzo[d]imidazol-5-yl)(cyclopropyl)methyl)-4,4-difluoro-3-methylbutanamide

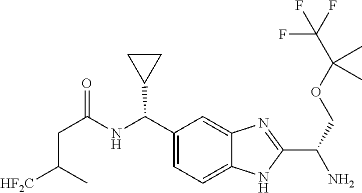

The title compound was prepared as described for the synthesis of Intermediate 197, using N-((1R)-(2-((1R)-1-((tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)-4,4-difluoro-3-methylbutanamide (Intermediate 227) in place of N—((R)-(2-((R)-1-(((R)-tert-butylsulfinyl)amino)-2-((1,1,1-trifluoro-2-methylpropan-2-yl)oxy)ethyl)-1-((2-(trimethylsilyl)ethoxy)methyl)-1H-benzo[d]imidazol-5-yl)(cyclopropyl)methyl)-2-(3,3-difluorocyclobutyl)acetamide. The product was used crude without further purification.

### Intermediate 229

### N-Phenyl-4-(2,2-difluoroethoxy)-1,2,5-oxadiazole-3-carboxamide

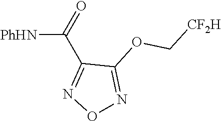

A round bottom flask was charged with NaH (60 wt % suspension in mineral oil, 1.6 g, 40 mmol) and THF (27 mL). Then, 2,2-difluoroethanol (0.85 mL, 13.4 mmol) was added dropwise to the NaH suspension at rt and stirred for 5 min. 4-Chloro-N-phenyl-1,2,5-oxadiazole-3-carboxamide (1.5 g, 6.7 mmol) was then added as a solid and the mixture was heated to 50° C. for 1.25 h. The reaction was then cooled to rt and quenched slowly with 1 N aqueous HCl. The aqueous layer was extracted three times with EtOAc and the combined organic layers were washed with brine, dried over anhydrous MgSO₄, filtered, and concentrated under reduced pressure. Purification by silica gel chromatography (0-25% EtOAc/hexanes) afforded the title compound as a pale-yellow solid.

### Intermediate 230

### tert-Butyl phenyl(4-(2,2-difluoroethoxy)-1,2,5-oxadiazole-3-carbonyl)carbamate

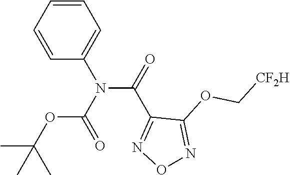 [SMILES: CC(C)(C)OC(=O)N(C(=O)c1nonc1OCC(F)F)c1ccccc1]

Di-tert-butyl dicarbonate (1.8 g, 8.4 mmol) and DMAP (81 mg, 0.67 mmol) were added sequentially to a solution of N-phenyl-4-(2,2-difluoroethoxy)-1,2,5-oxadiazole-3-carboxamide (1.8 g, 6.7 mmol, Intermediate 229) in DCM (33 mL) at rt and allowed to stir at that temperature for 2 h. Upon complete consumption of starting material, the reaction was quenched with a saturated aqueous solution of NaHCO₃ (20 mL) and transferred to a separatory funnel. The biphasic mixture was extracted with EtOAc (3×20 mL) and the combined organic layers were washed with brine, dried over anhydrous MgSO₄, filtered, and concentrated under reduced pressure to afford crude title compound, which was used without further purification.

### Intermediate 231

### 4-(2,2-Difluoroethoxy)-1,2,5-oxadiazole-3-carboxylic acid

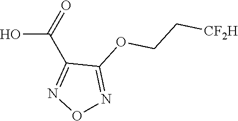 [SMILES: O=C(O)c1nonc1OCCC(F)F]

A solution of LiOH (270 mg, 11.4 mmol) in water (5.7 mL) was added dropwise to a solution of N-phenyl-4-(2,2-difluoroethoxy)-1,2,5-oxadiazole-3-carboxamide (2.5 g, 6.7 mmol, Intermediate 230) in THF (6.7 mL) and heated to 35° C. After 1.5 h, the reaction was acidified to pH 1 with 1 N aqueous HCl (10 mL) and extracted with EtOAc (3×5 mL). The combined organic layers were washed with a saturated aqueous solution of NaHCO₃ and the organic layer was discarded. The new aqueous layer was slowly acidified to pH 1 with 6 N aqueous HCl and extracted with EtOAc (3×20 mL). The new combined organic layers were dried over anhydrous Na₂SO₄, filtered, and concentrated under reduced pressure to afford the title compound as a hygroscopic solid. The title compound was diluted in MeCN and water, frozen, and lyophilized to dryness to generate a white powder.

### Intermediate 232

### (4,4-Difluorocyclohexyl)methanol

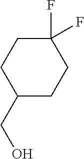

To a mixture of LAH (16.7 g, 439 mmol) in THF (1000 mL) was added a solution of 4,4-difluorocyclohexane-1-carboxylic acid (50.0 g, 305 mmol) in THF (1000 mL) at 5° C. The solution was stirred at 15° C. for 12 h, then the solution was quenched with water. The mixture was filtered, and the filter cake was washed with THF. The filtrate was concentrated in vacuo to yield the title compound as a yellow liquid which was used directly without purification.

### Intermediate 233

### 4,4-Difluorocyclohexane-1-carbaldehyde

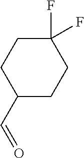

To a solution of (COCl)₂ (110 g, 866 mmol, 75.8 mL) in DCM (600 mL) was added DMSO (62.4 g, 799 mmol, 62.4 mL) in DCM (300 mL) at −78° C. After stirring at −78° C. for 30 min, a solution of (4,4-difluorocyclohexyl)methanol (100 g, 666 mmol, Intermediate 232) in DCM (500 mL) was added dropwise at −78° C. The mixture was stirred at −78° C. for 1 h then Et₃N (300 mL) was added at −20° C. followed by water (500 mL) at 0° C. The layers were separated and the organic layer was washed with water (3×500 mL), brine (500 mL), dried over anhydrous MgSO₄, filtered, and the filtrate was concentrated in vacuo to yield the title compound as a yellow liquid which was used directly without purification.

### Intermediate 234

### (R,Z)—N-((4,4-Difluorocyclohexyl)methylene)-2-methylpropane-2-sulfinamide

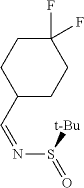 [SMILES: CC(C)(C)[S@@](=O)/N=C\C1CCC(F)(F)CC1]

PPTS (17.5 g, 69.5 mmol) was added to a solution of 4,4-difluorocyclohexane-1-carbaldehyde (103 g, 695 mmol, Intermediate 233), (R)-2-methylpropane-2-sulfinamide (84.3 g, 695 mmol) and CuSO₄ (333 g, 2.09 mol) in DCM (2000 mL). The solution was stirred for 15 h at 15° C. The mixture was filtered and the filtrate was concentrated in vacuo. The crude material was purified by silica gel chromatography (6-10% EtOAc/petroleum ether) to provide the title compound as a yellow oil.

### Intermediate 235

### 5-Bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole

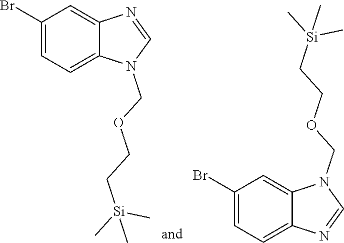 [SMILES: C[Si](C)(C)CCOCn1cnc2cc(Br)ccc21 ; C[Si](C)(C)CCOCn1cnc2ccc(Br)cc21]

To a solution of 5-bromo-1H-benzo[d]imidazole (50.0 g, 253 mmol) in DMF (500 mL) was added NaH (16.2 g, 406 mmol, 60.0% purity) and the resulting mixture was stirred at 0° C. for 1 h. Then the purple mixture was cooled to 0° C. and SEM-Cl (46.5 g, 279 mmol, 49.4 mL) was added in portions over 1 h. The yellow mixture was then allowed to slowly warm to 20° C. then stirred at that temperature for 12 h. The reaction mixture was partitioned between EtOAc (500 mL) and water (500 mL). The layers were separated, and the aqueous layer was further extracted with EtOAc (2×200 mL). The combined organic layers were washed with brine (2×500 mL), dried over anhydrous sodium sulfate, filtered, and concentrated under reduced pressure. The residue was purified by silica gel chromatography (9-50% EtOAc/petroleum ether) to provide the mixture of title compounds as a yellow oil.

### Intermediate 236

### 1-((2-(Trimethylsilyl)ethoxy)methyl)-5-vinyl-1H-benzo[d]imidazole and 1-((2-(trimethylsilyl)ethoxy)methyl)-6-vinyl-1H-benzo[d]imidazole

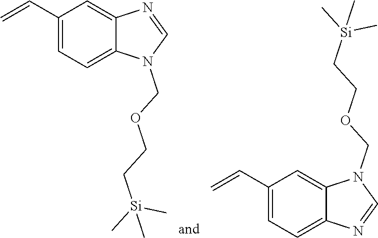 [SMILES: C=Cc1ccc2c(c1)ncn2COCC[Si](C)(C)C ; C=Cc1ccc2ncn(COCC[Si](C)(C)C)c2c1]

A solution of 1,4-dioxane (600 mL) and water (100 mL) was sparged with nitrogen for 10 min, followed by the addition of 5-bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole (50.0 g, 153 mmol, Intermediate 235), potassium trifluoro(vinyl)boranide (40.9 g, 305 mmol), K₃PO₄ (97.3 g, 458 mmol) and [1,1′-bis(diphenylphosphino)ferrocene]dichloropalladium(II) complex with dichloromethane (6.24 g, 7.64 mmol). The brown mixture was stirred at 85° C. for 3 h. Then the reaction mixture was cooled to rt, filtered, and concentrated under reduced pressure. The residue was dissolved in H₂O (300 mL), extracted with DCM (3×200 mL) and washed with brine (2×500 mL). The combined organic layers were dried over anhydrous sodium sulfate, filtered, and concentrated under reduced pressure to provide the mixture of title compounds as a black oil which was used directly without purification.

### Intermediate 237

### 1-((2-(Trimethylsilyl)ethoxy)methyl)-1H-benzo[d]imidazole-5-carbaldehyde and 1-((2-(trimethylsilyl)ethoxy)methyl)-1H-benzo[d]imidazole-6-carbaldehyde

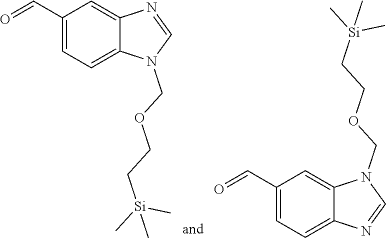 [SMILES: C[Si](C)(C)CCOCn1cnc2cc(C=O)ccc21 ; C[Si](C)(C)CCOCn1cnc2ccc(C=O)cc21]

To a solution of 1-((2-(trimethylsilyl)ethoxy)methyl)-5-vinyl-1H-benzo[d]imidazole and 1-((2-(trimethylsilyl)ethoxy)methyl)-6-vinyl-1H-benzo[d]imidazole (45.0 g, 164 mmol, Intermediate 236) in 1,4-dioxane (800 mL) and H₂O (800 mL) was added K₂OsO₄·2H₂O (2.42 g, 6.56 mmol) and NaIO₄ (105 g, 492 mmol, 27.3 mL). The yellow suspension was stirred at 25° C. for 12 h. Then the reaction mixture was filtered and washed with EtOAc (100 mL). The filtrate was extracted with EtOAc (2×400 mL) and washed with brine (2×500 mL). The combined organic layers were dried over anhydrous sodium sulfate, filtered, and concentrated under reduced pressure. The residue was purified by silica gel chromatography (9-50% EtOAc/petroleum ether) to provide the mixture of title compounds as a yellow oil.

### Intermediate 238

### (S,E)-2-Methyl-N-((1-((2-(trimethylsilyl)ethoxy)methyl)-1H-benzo[d]imidazol-5-yl)methylene)propane-2-sulfinamide and (S,E)-2-methyl-N-((1-((2-(trimethylsilyl)ethoxy)methyl)-1H-benzo[d]imidazol-6-yl)methylene)propane-2-sulfinamide

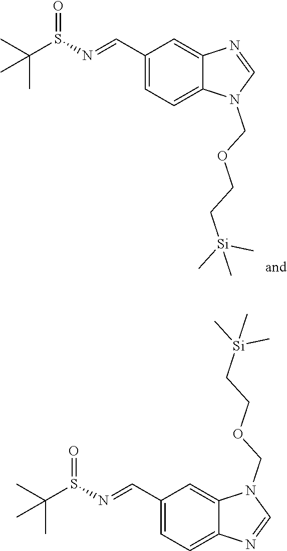

1-((2-(Trimethylsilyl)ethoxy)methyl)-1H-benzo[d]imidazole-5-carbaldehyde and 1-((2-(trimethylsilyl)ethoxy)methyl)-1H-benzo[d]imidazole-6-carbaldehyde (60.8 g, 220 mmol, Intermediate 237), 2-methylpropane-2-sulfinamide (40.0 g, 330 mmol), CuSO₄ (105 g, 660 mmol, 101 mL) and DCM (300 mL) were combined followed by the addition of PPTS (5.53 g, 22.0 mmol). The blue mixture was stirred at 35° C. for 12 h. After that time, the reaction mixture was filtered, and the filter cake was washed with DCM (100 mL). The filtrate was washed with brine (2×400 mL), and then the combined organic layers were dried over anhydrous Na₂SO₄, filtered, and concentrated to dryness. The residue was purified by silica gel chromatography (9-50% EtOAc/petroleum ether) to provide the mixture of title compounds as a yellow gum.

### Intermediate 239

### (S)-2-Methyl-N—((R)-1-(1-((2-(trimethylsilyl)ethoxy)methyl)-1H-benzo[d]imidazol-5-yl)ethyl)propane-2-sulfinamide and (S)-2-methyl-N—((R)-1-(1-((2-(trimethylsilyl)ethoxy)methyl)-1H-benzo[d]imidazol-6-yl)ethyl)propane-2-sulfinamide

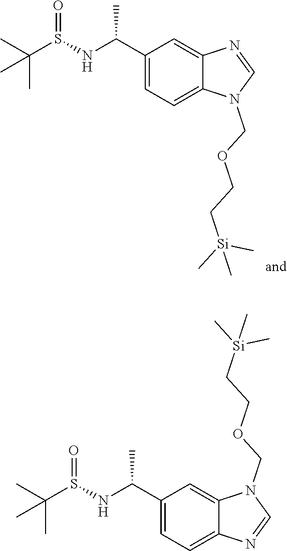

To a mixture of (S,E)-2-methyl-N-((1-((2-(trimethylsilyl)ethoxy)methyl)-1H-benzo[d]imidazol-5-yl)methylene)propane-2-sulfinamide and (S,E)-2-methyl-N-((1-((2-(trimethylsilyl)ethoxy)methyl)-1H-benzo[d]imidazol-6-yl)methylene)propane-2-sulfinamide (26.0 g, 68.5 mmol, Intermediate 238) in DCM (260 mL) at −70° C. was added methyl magnesium bromide (73.50 g, 616 mmol). The brown mixture was warmed to 20° C. gradually and stirred for 12 h. The reaction mixture was quenched by the addition of saturated aqueous NH₄Cl. The crude reaction mixture was extracted with EtOAc (2×500 mL) and the combined organic layers were washed with brine (2×800 mL), dried over anhydrous Na₂SO₄, filtered and concentrated under reduced pressure. The residue was purified by silica gel chromatography (25-100% EtOAc/petroleum ether) to provide the mixture of title compounds as a yellow solid, which was further purified by re-crystallization from MTBE (100 mL) and petroleum ether (400 mL) at 20° C.

### Intermediate 240

### (S)—N-(Cyclopropyl(1-((2-(trimethylsilyl)ethoxy)methyl)-1H-benzo[d]imidazol-5-yl)methyl)-2-methylpropane-2-sulfinamide and (S)—N-(cyclopropyl(1-((2-(trimethylsilyl)ethoxy)methyl)-1H-benzo[d]imidazol-6-yl)methyl)-2-methylpropane-2-sulfinamide

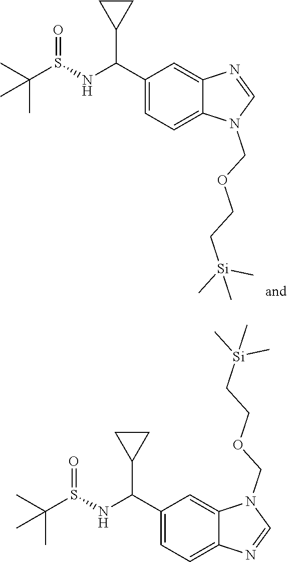

The title compounds were prepared as described for the synthesis of Intermediate 239, using cyclopropyl magnesium bromide in place of methyl magnesium bromide to provide the mixture of title compounds.

### Intermediate 241

### (S,E)-2-Methyl-N-((1-((2-(trimethylsilyl)ethoxy)methyl)-1H-benzo[d]imidazol-6-yl)methylene)propane-2-sulfinamide

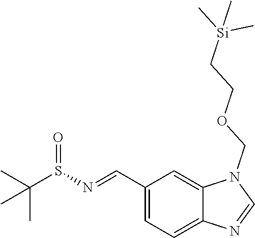 [SMILES: CC(C)(C)[S@](=O)/N=C/c1ccc2ncn(COCC[Si](C)(C)C)c2c1]

### Intermediate 242

### (S,E)-2-Methyl-N-((1-((2-(trimethylsilyl)ethoxy)methyl)-1H-benzo[d]imidazol-5-yl)methylene)propane-2-sulfinamide

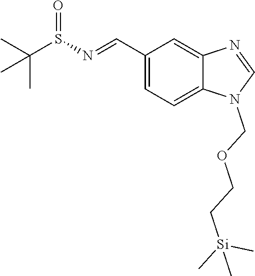 [SMILES: CC(C)(C)[S@](=O)/N=C/c1ccc2c(c1)ncn2COCC[Si](C)(C)C]

(S,E)-2-Methyl-N-((1-((2-(trimethylsilyl)ethoxy)methyl)-1H-benzo[d]imidazol-5-yl)methylene)propane-2-sulfinamide and (S,E)-2-methyl-N-((1-((2-(trimethylsilyl)ethoxy)methyl)-1H-benzo[d]imidazol-6-yl)methylene)propane-2-sulfinamide (Intermediate 238) were purified by silica gel chromatography (15-50% EtOAc/petroleum ether) to provide two SEM regioisomers, Intermediate 241 and Intermediate 242, as yellow oils.

### Intermediate 243

### (S)—N—((R)-Cyclopropyl(1-((2-(trimethylsilyl)ethoxy)methyl)-1H-benzo[d]imidazol-5-yl)methyl)-2-methylpropane-2-sulfinamide

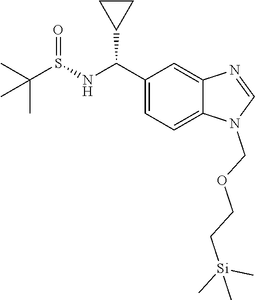 [SMILES: CC(C)(C)[S@](=O)N[C@@H](c1ccc2c(c1)ncn2COCC[Si](C)(C)C)C1CC1]

Bromo(cyclopropyl)magnesium (1 M in THF, 1.30 L, 1.3 mol) was added to the mixture of (S,E)-2-methyl-N-((1-((2-(trimethylsilyl)ethoxy)methyl)-1H-benzo[d]imidazol-6-yl)methylene)propane-2-sulfinamide (60.0 g, 144 mmol, Intermediate 242) in DCM (600 mL) at −70° C. under a N2 atmosphere. The brown mixture was warmed to 20° C. gradually and stirred for 12 h. The reaction mixture was quenched by the addition of saturated aqueous NH₄Cl (1000 mL) and extracted with EtOAc (2×1000 mL). The combined organic layers were washed with brine (1000 mL), dried over anhydrous Na₂SO₄, filtered, and concentrated to dryness under reduced pressure. The residue was purified by silica gel chromatography (50-100% EtOAc/petroleum ether) then repurified by preparative HPLC (YMC—Triart Pre C18, 250×50 mm, 5-95% ACN/H₂O with 0.5% NH₄OH) to obtain the title compound as a yellow solid.

### Intermediate 244

### (S)—N—((R)-Cyclopropyl(1-((2-(trimethylsilyl)ethoxy)methyl)-1H-benzo[d]imidazol-6-yl)methyl)-2-methylpropane-2-sulfinamide

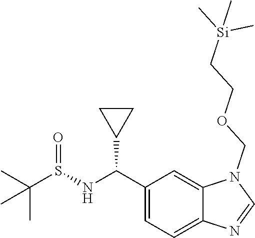 [SMILES: CC(C)(C)[S@](=O)N[C@@H](c1ccc2ncn(COCC[Si](C)(C)C)c2c1)C1CC1]

The title compound was prepared as described for the synthesis of Intermediate 243, using (S,E)-2-methyl-N-((1-((2-(trimethylsilyl)ethoxy)methyl)-1H-benzo[d]imidazol-6-yl)methylene)propane-2-sulfinamide (Intermediate 241) in place of (S,E)-2-methyl-N-((1-((2-(trimethylsilyl)ethoxy)methyl)-1H-benzo[d]imidazol-5-yl)methylene)propane-2-sulfinamide, to provide the title compound.

### Intermediate 245

### (S)—N-((4,4-Difluorocyclohexyl)(5-formyl-1H-benzo[d]imidazol-2-yl)methyl)-1-methyl-1H-pyrazole-5-carboxamide

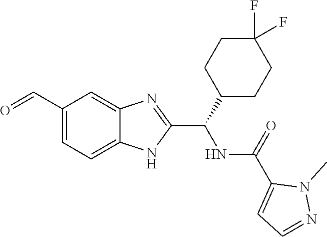 [SMILES: Cn1nccc1C(=O)N[C@H](c1nc2cc(C=O)ccc2[nH]1)C1CCC(F)(F)CC1]

(S)—N-((5-Cyano-1H-benzo[d]imidazol-2-yl)(4,4-difluorocyclohexyl)methyl)-1-methyl-1H-pyrazole-5-carboxamide (3.68 g, 9.23 mmol, Intermediate 64), pyridine (22 mL) and acetic acid (11 mL) were combined and stirred at rt. Then, Raney®-Nickel (1.45 g, 12.4 mmol, ˜3 mL slurry in water) was added to the reaction mixture followed by sodium hypophosphite monohydrate (6.57 g, 62.0 mmol) in water (11 mL), then the reaction vessel was heated to 50 for 3 h. The contents were cooled and filtered through Celite® and washed with EtOAc. The filtrate was concentrated to provide the title compound that was used in the subsequent reaction without further purification.

### Intermediate 246

### N—((S)-(5-((E)-(((S)-tert-Butylsulfinyl)imino)methyl)-1H-benzo[d]imidazol-2-yl)(4,4-difluorocyclohexyl)methyl)-1-methyl-1H-pyrazole-5-carboxamide

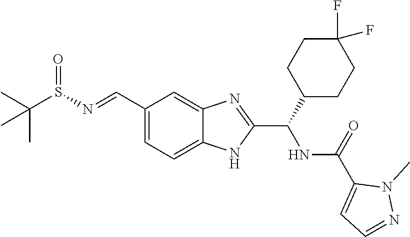

(S)—N-((4,4-Difluorocyclohexyl)(5-formyl-1H-benzo[d]imidazol-2-yl)methyl)-1-methyl-1H-pyrazole-5-carboxamide (7.6 g, 9.47 mmol, Intermediate 245), THF (50 mL), (S)-2-methylpropane-2-sulfinamide (2.34 g, 19.3 mmol), copper(II) sulfate (7.57 g, 47.4 mmol) and pyridine 4-methylbenzenesulfonate (672 mg, 2.67 mmol) were combined and heated at 65° C. overnight. The contents were cooled and filtered through Celite® with liberal EtOAc washing. The filtrate was concentrated, and the residue was purified by silica gel chromatography (0-100% (10% (2 M NH₃ in MeOH) in DCM)/DCM) to provide the title compound.

### Intermediate 247

### N—((S)-(5-((R)-1-(((S)-tert-Butylsulfinyl)amino)ethyl)-1H-benzo[d]imidazol-2-yl)(4,4-difluorocyclohexyl)methyl)-1-methyl-1H-pyrazole-5-carboxamide

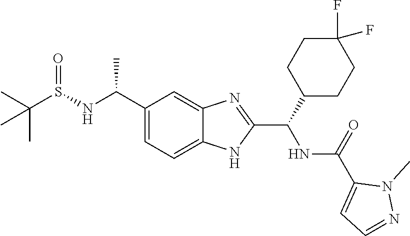 [SMILES: C[C@@H](N[S@@](=O)C(C)(C)C)c1ccc2[nH]c([C@@H](NC(=O)c3ccnn3C)C3CCC(F)(F)CC3)nc2c1]

The title compound was prepared as described for the synthesis of Intermediate 239, using N—((S)-(5-((E)-(((S)-tert-butylsulfinyl)imino)methyl)-1H-benzo[d]imidazol-2-yl)(4,4-difluorocyclohexyl)methyl)-1-methyl-1H-pyrazole-5-carboxamide (Intermediate 246) in place of (S,E)-2-methyl-N-((1-((2-(trimethylsilyl)ethoxy)methyl)-1H-benzo[d]imidazol-5-yl)methylene)propane-2-sulfinamide and (S,E)-2-methyl-N-((1-((2-(trimethylsilyl)ethoxy)methyl)-1H-benzo[d]imidazol-6-yl)methylene)propane-2-sulfinamide, to provide the title compound.

### Intermediate 248

### (R)-Cyclopropyl(1-((2-(trimethylsilyl)ethoxy)methyl)-1H-benzo[d]imidazol-5-yl)methanamine

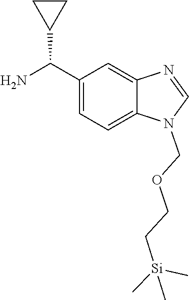 [SMILES: C[Si](C)(C)CCOCn1cnc2cc([C@H](N)C3CC3)ccc21]

To a solution of (S)—N—((R)-cyclopropyl(1-((2-(trimethylsilyl)ethoxy)methyl)-1H-benzo[d]imidazol-5-yl)methyl)-2-methylpropane-2-sulfinamide (1.00 g, 2.37 mmol, Intermediate 243) in EtOAc (22 mL) was added 4 M HCl in 1,4-dioxane (3.0 mL, 12 mmol) at 0° C. under nitrogen. The mixture was stirred at 0° C. for 30 min, then the mixture was warmed to rt and stirred for 16 h. To the reaction was added petroleum ether (100 mL) and the mixture was filtered. To the filtered solids was added aqueous NaHCO₃ solution, then the mixture was extracted with EtOAc (3×50 mL). The combined organic layers were dried over anhydrous Na₂SO₄, filtered, and concentrated under reduced pressure to provide the title compound as a yellow oil.

### Intermediate 249

### Cyclopropyl(1-((2-(trimethylsilyl)ethoxy)methyl)-1H-benzo[d]imidazol-5-yl)methanamine and cyclopropyl(1-((2-(trimethylsilyl)ethoxy) methyl)-1H-benzo[d]imidazol-6-yl)methanamine

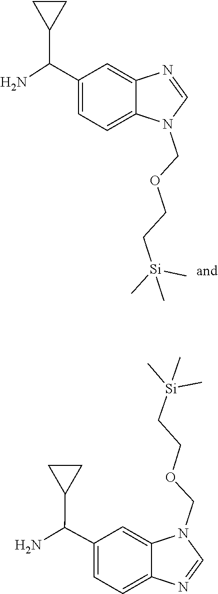 [SMILES: C[Si](C)(C)CCOCn1cnc2cc(C(N)C3CC3)ccc21 ; C[Si](C)(C)CCOCn1cnc2ccc(C(N)C3CC3)cc21]

The title compound was prepared as described for the synthesis of Intermediate 186, using (S)—N-(cyclopropyl(1-((2-(trimethylsilyl)ethoxy)methyl)-1H-benzo[d]imidazol-5-yl)methyl)-2-methylpropane-2-sulfinamide and (S)—N-(cyclopropyl(1-((2-(trimethylsilyl)ethoxy)methyl)-1H-benzo[d]imidazol-6-yl)methyl)-2-methylpropane-2-sulfinamide (Intermediate 240) in place of (R)-cyclopropyl(1-((2-(trimethylsilyl)ethoxy)methyl)-1H-benzo[d]imidazol-5-yl)methanamine to provide the title compound.

### Intermediate 250

### (R,E)-2-Methyl-N-(4,4,4-trifluoro-3,3-dimethylbutylidene)propane-2-sulfinamide

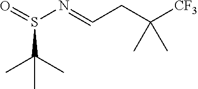 [SMILES: CC(C)(C)[S@@](=O)/N=C/CC(C)(C)C(F)(F)F]

To a solution of 3,3,3-trifluoropropanal (140.0 g, 908.3 mmol) in DCM (1500 mL) was added (R)-2-methylpropane-2-sulfinamide (132.1 g, 1.09 mol) and PPTS (23.1 g, 91.7 mmol) and CuSO₄ (430 g, 2.74 mol), then the reaction was stirred for 12 h at 30° C. The reaction was filtered through Celite®, then the filtrate was concentrated under reduced pressure to give a yellow oil. The yellow oil was purified by silica gel chromatography (0-10% EtOAc/petroleum ether) to obtain the title compound as a yellow oil.

### Intermediate 251

### (R)—N—((S)-1-(5-((R)-Amino(cyclopropyl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide

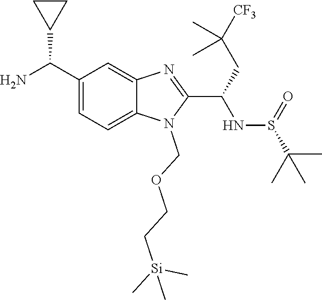 [SMILES: CC(C)(C)[S@@](=O)N[C@@H](CC(C)(C)C(F)(F)F)c1nc2cc([C@H](N)C3CC3)ccc2n1COCC[Si](C)(C)C]

A mixture of (R)-cyclopropyl(1-((2-(trimethylsilyl)ethoxy)methyl)-1H-benzo[d]imidazol-5-yl)methanamine (300 mg, 0.945 mmol, Intermediate 248) in THF (5 mL) was cooled to −78° C. under a nitrogen atmosphere. Then, n-BuLi in hexanes (2.5 M, 1.13 mL, 2.84 mmol) was added and the reaction was stirred for 2 h at −78° C. A solution of (R,E)-2-methyl-N-(4,4,4-trifluoro-3,3-dimethylbutylidene)propane-2-sulfinamide (608 mg, 2.36 mmol, Intermediate 250) in THF (5 mL) was added slowly and the contents allowed to warm to rt gradually, then stirred at rt for 16 h. The reaction was washed with saturated aqueous NH₄Cl solution (30 mL), then the aqueous layer was further extracted with DCM (2×50 mL). The combined organic phases were dried over anhydrous Na₂SO₄, filtered, and concentrated under reduced pressure. The residue was purified by silica gel chromatography (0-10% MeOH/DCM) to obtain the title compound as a yellow oil.

### Intermediate 252

### (R)—N—((S)-1-(6-((R)-Amino(cyclopropyl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide

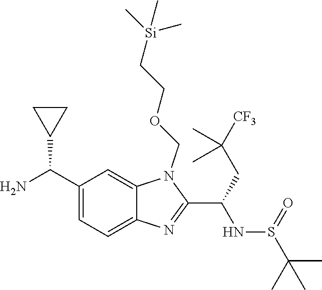 [SMILES: CC(C)(C)S(=O)N[C@@H](CC(C)(C)C(F)(F)F)c1nc2ccc([C@H](N)C3CC3)cc2n1COCC[Si](C)(C)C]

The title compound was prepared as described for the synthesis of Intermediate 251, using (R)-cyclopropyl(1-((2-(trimethylsilyl)ethoxy)methyl)-1H-benzo[d]imidazol-6-yl)methanamine (Intermediate 186) in place of (R)-cyclopropyl(1-((2-(trimethylsilyl)ethoxy)methyl)-1H-benzo[d]imidazol-5-yl)methanamine to provide the title compound.

### Intermediate 253

### N—((R)-(2-((S)-1-(((R)-tert-Butylsulfinyl)amino)-4,4,4-trifluoro-3,3-dimethylbutyl)-1-((2-(trimethylsilyl)ethoxy)methyl)-1H-benzo[d]imidazol-5-yl)(cyclopropyl)methyl)-2-(3,3-difluorocyclobutyl)acetamide

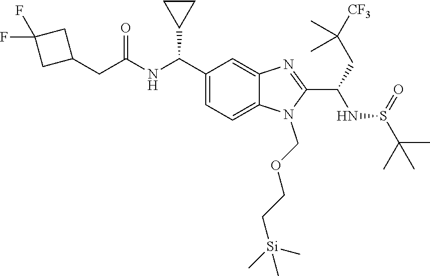 [SMILES: CC(C)(C)[S@@](=O)N[C@@H](CC(C)(C)C(F)(F)F)c1nc2cc([C@H](NC(=O)CC3CC(F)(F)C3)C3CC3)ccc2n1COCC[Si](C)(C)C]

A solution of (R)—N—((S)-1-(5-((R)-amino(cyclopropyl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide (150 mg, 0.26 mmol, Intermediate 251) in CH₃CN (2 mL) was added dropwise to a stirred solution of 2-(3,3-difluorocyclobutyl)acetic acid (78.4 mg, 0.522 mmol), HOBt (42.3 mg, 0.313 mmol), EDCI (100 mg, 0.522 mmol) and DIPEA (0.091 mL, 0.522 mmol) and the resultant mixture was stirred at 25° C. for 16 h. The crude reaction mixture was diluted with water and extracted with DCM. The combined organic layers were washed with brine, dried over anhydrous Na₂SO₄, filtered, and concentrated under reduced pressure. The residue was purified by preparative TLC (0-50% EtOAc/petroleum ether) to afford the title compound.

### Intermediate 254

### N—((R)-(2-((S)-1-(((R)-tert-Butylsulfinyl)amino)-4,4,4-trifluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide

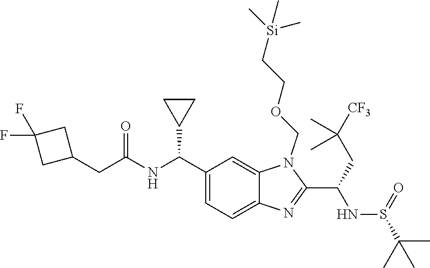 [SMILES: CC(C)(C)[S@@](=O)N[C@@H](CC(C)(C)C(F)(F)F)c1nc2ccc([C@H](NC(=O)CC3CC(F)(F)C3)C3CC3)cc2n1COCC[Si](C)(C)C]

The title compound was prepared as described for the synthesis of Intermediate 253, using (R)—N—((S)-1-(6-((R)-amino(cyclopropyl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide (Intermediate 252) in place of N—((R)-(2-((S)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-5-yl)(cyclopropyl)methyl)-2-(3,3-difluorocyclobutyl)acetamide to provide the title compound.

### Intermediate 255

### N—((R)-(2-((S)-1-Amino-4,4,4-trifluoro-3,3-dimethylbutyl)-1H-benzo[d]imidazol-6-yl)(cyclopropyl)methyl)-2-(3,3-difluorocyclobutyl)acetamide

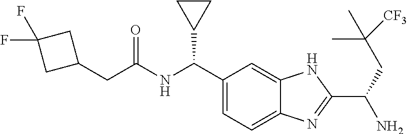 [SMILES: CC(C)(C[C@H](N)c1nc2ccc([C@H](NC(=O)CC3CC(F)(F)C3)C3CC3)cc2[nH]1)C(F)(F)F]

A solution of HCl in 1,4-dioxane (4 M, 4.21 mL, 16.8 mmol) was added to a mixture of N—((R)-cyclopropyl(2-((S)-1-((R)-1,1-dimethylethylsulfinamido)-4,4,4-trifluoro-3,3-dimethylbutyl)-1-((2-(trimethylsilyl)ethoxy)methyl)-1H-benzo[d]imidazol-6-yl)methyl)-2-(3,3-difluorocyclobutyl)acetamide (245 mg, 0.347 mmol, Intermediate 254) in 1,4-dioxane (6 mL). After addition, the mixture was stirred at 55° C. for 2.5 h. The reaction was cooled to rt, petroleum ether (20 mL) was added, and the slurry was stirred for 10 min then extracted with H₂O (20 mL). The aqueous phase was adjusted to pH=9 with 1 M aqueous NaHCO₃, then extracted with DCM (2×20 mL). The combined organic layers were washed sequentially with water (30 mL) and brine (30 mL), then dried over anhydrous Na₂SO₄, filtered, and concentrated in vacuo to give the title compound.

### Intermediate 256

### N—((R)-(2-((S)-1-(((R)-tert-Butylsulfinyl)amino)-4,4,4-trifluoro-3,3-dimethylbutyl)-1-((2-(trimethylsilyl)ethoxy)methyl)-1H-benzo[d]imidazol-5-yl)(cyclopropyl)methyl)-4,4,4-trifluoro-3-methylbutanamide

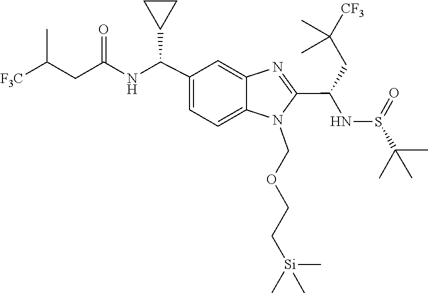 [SMILES: CC(CC(=O)N[C@@H](c1ccc2c(c1)nc([C@H](CC(C)(C)C(F)(F)F)N[S@](=O)C(C)(C)C)n2COCC[Si](C)(C)C)C1CC1)C(F)(F)F]

The title compound was prepared as described for the synthesis of Intermediate 253, using 4,4,4-trifluoro-3-methylbutanoic acid in place of 2-(3,3-difluorocyclobutyl)acetic acid, to provide the title compound.

### Intermediate 257

### N—((R)-(2-((S)-1-Amino-4,4,4-trifluoro-3,3-dimethylbutyl)-1H-benzo[d]imidazol-6-yl)(cyclopropyl)methyl)-4,4,4-trifluoro-3-methylbutanamide

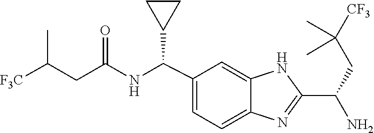 [SMILES: CC(CC(=O)N[C@@H](c1ccc2nc([C@@H](N)CC(C)(C)C(F)(F)F)[nH]c2c1)C1CC1)C(F)(F)F]

The title compound was prepared as described for the synthesis of Intermediate 255, using N—((R)-(2-((S)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-5-yl)(cyclopropyl)methyl)-4,4,4-trifluoro-3-methylbutanamide (Intermediate 256) in place of N—((R)-cyclopropyl(2-((S)-1-((R)-1,1-dimethylethylsulfinamido)-4,4,4-trifluoro-3,3-dimethylbutyl)-1-((2-(trimethylsilyl)ethoxy)methyl)-1H-benzo[d]imidazol-6-yl)methyl)-2-(3,3-difluorocyclobutyl)acetamide, to provide the title compound.

### Intermediate 258

### N—((R)-(2-((S)-1-(((R)-tert-Butylsulfinyl)amino)-4,4,4-trifluoro-3,3-dimethylbutyl)-1-((2-(trimethylsilyl)ethoxy)methyl)-1H-benzo[d]imidazol-6-yl)(cyclopropyl)methyl)-2-(2,2-difluorocyclopropyl)acetamide

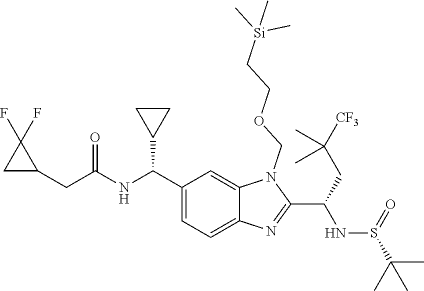 [SMILES: CC(C)(C)[S@@](=O)N[C@@H](CC(C)(C)C(F)(F)F)c1nc2ccc([C@H](NC(=O)CC3CC3(F)F)C3CC3)cc2n1COCC[Si](C)(C)C]

The title compound was prepared as described for the synthesis of Intermediate 253, using 2-(2,2-difluorocyclopropyl)acetic acid in place of 2-(3,3-difluorocyclobutyl)acetic acid, to provide the title compound.

### Intermediate 259

### N—((R)-(2-((S)-1-Amino-4,4,4-trifluoro-3,3-dimethylbutyl)-1H-benzo[d]imidazol-6-yl)(cyclopropyl)methyl)-2-(2,2-difluorocyclopropyl)acetamide

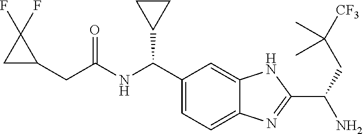 [SMILES: CC(C)(C[C@H](N)c1nc2ccc([C@H](NC(=O)CC3CC3(F)F)C3CC3)cc2[nH]1)C(F)(F)F]

The title compound was prepared as described for the synthesis of Intermediate 255, using N—((R)-(2-((S)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-6-yl)(cyclopropyl)methyl)-2-(2,2-difluorocyclopropyl)acetamide (Intermediate 258) in place of N—((R)-cyclopropyl(2-((S)-1-((R)-1,1-dimethylethylsulfinamido)-4,4,4-trifluoro-3,3-dimethylbutyl)-1-((2-(trimethylsilyl)ethoxy)methyl)-1H-benzo[d]imidazol-6-yl)methyl)-2-(3,3-difluorocyclobutyl)acetamide, to provide the title compound.

### Intermediate 260

### N—((R)-(2-((S)-1-(((R)-tert-Butylsulfinyl)amino)-4,4,4-trifluoro-3,3-dimethylbutyl)-1-((2-(trimethylsilyl)ethoxy)methyl)-1H-benzo[d]imidazol-6-yl)(cyclopropyl)methyl)-4,4-difluorobutanamide

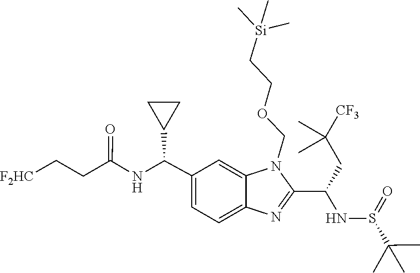 [SMILES: CC(C)(C)[S@@](=O)N[C@@H](CC(C)(C)C(F)(F)F)c1nc2ccc([C@H](NC(=O)CCC(F)F)C3CC3)cc2n1COCC[Si](C)(C)C]

The title compound was prepared as described for the synthesis of Intermediate 253, using 4,4-difluorobutanoic acid in place of 2-(3,3-difluorocyclobutyl)acetic acid, to provide the title compound.

### Intermediate 261

### N—((R)-(2-((S)-1-Amino-4,4,4-trifluoro-3,3-dimethylbutyl)-1H-benzo[d]imidazol-6-yl)(cyclopropyl)methyl)-4,4-difluorobutanamide

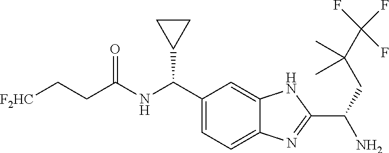 [SMILES: CC(C)(C[C@H](N)c1nc2ccc([C@H](NC(=O)CCC(F)F)C3CC3)cc2[nH]1)C(F)(F)F]

The title compound was prepared as described for the synthesis of Intermediate 255, using N—((R)-(2-((S)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-6-yl)(cyclopropyl)methyl)-4,4-difluorobutanamide (Intermediate 260) in place of N—((R)-cyclopropyl(2-((S)-1-((R)-1,1-dimethylethylsulfinamido)-4,4,4-trifluoro-3,3-dimethylbutyl)-1-((2-(trimethylsilyl)ethoxy)methyl)-1H-benzo[d]imidazol-6-yl)methyl)-2-(3,3-difluorocyclobutyl)acetamide, to provide the title compound.

### Intermediate 262

### (R)-2-(Cyclopropyl(1-((2-(trimethylsilyl)ethoxy)methyl)-1H-benzo[d]imidazol-5-yl)methyl)isoindoline-1,3-dione

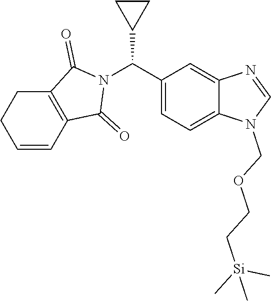 [SMILES: C[Si](C)(C)CCOCn1cnc2cc([C@@H](C3CC3)N3C(=O)C4=C(CCC=C4)C3=O)ccc21]

(R)-Cyclopropyl(1-((2-(trimethylsilyl)ethoxy)methyl)-1H-benzo[d]imidazol-5-yl)methanamine (13.2 g, 37.29 mmol, Intermediate 248) and THF (250 mL, 0.2 M, 50 mmol) were combined and stirred at rt under nitrogen followed by the addition of Hunig's base (20 mL, 116 mmol). The contents were stirred for 5 min at rt then ethyl 1,3-dioxoisoindoline-2-carboxylate (8.57 g, 39.1 mmol) was added, a reflux condenser connected, and the contents heated to reflux for 2 days. The contents were cooled to rt and transferred to a separatory funnel with EtOAc dilution, then washed 2× with deionized water. The organic phase was separated, dried over anhydrous magnesium sulfate, filtered, and concentrated under reduced pressure. Purification by silica gel chromatography (0-100% EtOAc/hexanes) yielded the title compound.

### Intermediate 263

### (R)-2-(1-(1-((2-(Trimethylsilyl)ethoxy)methyl)-1H-benzo[d]imidazol-5-yl)ethyl)isoindoline-1,3-dione and (R)-2-(1-(1-((2-(trimethylsilyl)ethoxy)methyl)-1H-benzo[d]imidazol-6-yl)ethyl)isoindoline-1,3-dione

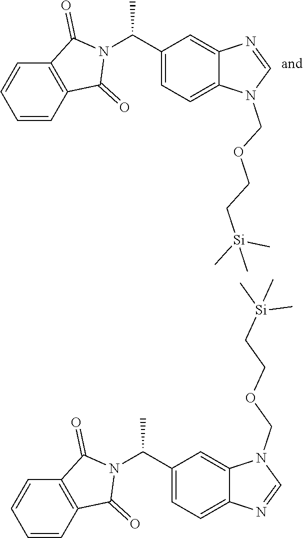

The title compound was prepared as described for the synthesis of Intermediate 262, using (R)-1-(1-((2-(trimethylsilyl)ethoxy)methyl)-1H-benzo[d]imidazol-6-yl)ethan-1-amine and (R)-1-(1-((2-(trimethylsilyl)ethoxy)methyl)-1H-benzo[d]imidazol-5-yl)ethan-1-amine (Intermediate 182) in place of (R)-cyclopropyl(1-((2-(trimethylsilyl)ethoxy)methyl)-1H-benzo[d]imidazol-5-yl)methanamine to provide the title compound.

### Intermediate 264

### (R)—N—((S)-1-(5-((R)-Cyclopropyl(1,3-dioxoisoindolin-2-yl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide

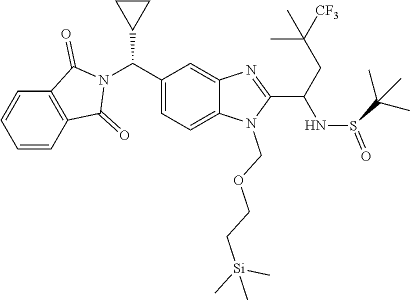 [SMILES: CC(C)(C)[S@@](=O)NC(CC(C)(C)C(F)(F)F)c1nc2cc([C@@H](C3CC3)N3C(=O)c4ccccc4C3=O)ccc2n1COCC[Si](C)(C)C]

(R)-2-(Cyclopropyl(1-((2-(trimethylsilyl)ethoxy)methyl)-1H-benzo[d]imidazol-5-yl)methyl)isoindoline-1,3-dione (504.4 mg, 1.13 mmol, Intermediate 262), (R,Z)-2-methyl-N-(4,4,4-trifluoro-3,3-dimethylbutylidene)propane-2-sulfinamide (503 mg, 1.76 mmol, Intermediate 250) and THF (6 mL) were combined and cooled to −78° C. LDA (1 M in hexanes/THF, 2.4 mL, 2.4 mmol) was then added dropwise over approximately 3 min. The contents were stirred at −78° C. for 2 h, then additional LDA (1 M in hexanes/THF, 2.4 mL, 2.4 mmol) was added. The reaction was stirred for 30 min at −78° C., then quenched with acetic acid. Then the ice bath was removed, and the contents warmed to rt. The contents were then transferred to a separatory funnel with EtOAc and extracted twice with deionized water. The organic phase was then separated, dried over anhydrous magnesium sulfate, filtered, and concentrated under reduced pressure. Purification by silica gel chromatography (0-100% EtOAc/hexanes) yielded the title compound.

### Intermediate 265

### (R)—N—((S)-1-(5-((R)-Amino(cyclopropyl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide

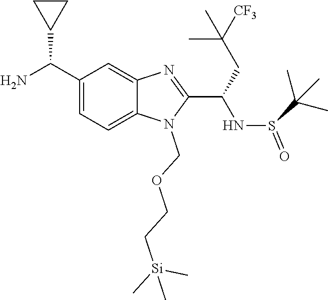 [SMILES: CC(C)(C)[S@@](=O)N[C@@H](CC(C)(C)C(F)(F)F)c1nc2cc([C@H](N)C3CC3)ccc2n1COCC[Si](C)(C)C]

(R)—N—((S)-1-(5-((R)-Cyclopropyl(1,3-dioxoisoindolin-2-yl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide (103.4 mg, 0.15 mmol, Intermediate 264), ethanol (2 mL) and hydrazine monohydrate (110 μL, 1.03 g/mL, 1.47 mmol) were combined and stirred at rt overnight. The contents were transferred to a separatory funnel with EtOAc and deionized water. The organic phase was separated, and the aqueous phase was salted with NaCl then extracted twice with EtOAc. The combined organic layers were dried over anhydrous magnesium sulfate, filtered, and concentrated under reduced pressure. Purification by silica gel chromatography (0-100% (10% 2 M NH₃/MeOH in DCM)/DCM) yielded the title compound.

### Intermediate 266

### (R)-Cyclopropyl(1-((2-(trimethylsilyl)ethoxy)methyl)-1H-benzo[d]imidazol-5-yl)methanamine hydrochloride

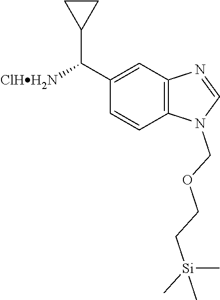

To a stirred solution of (S)—N—((R)-cyclopropyl(1-((2-(trimethylsilyl)ethoxy)methyl)-1H-benzo[d]imidazol-5-yl)methyl)-2-methylpropane-2-sulfinamide Intermediate 243 (60.0 g, 142 mmol) in EtOAc (983 ml) was added 4 M HCl in 1,4-dioxane (118 ml) in a dropwise manner over 30 min. After 24 h, the reaction mixture was diluted with ethyl ether, stirred for 2 h, filtered to collect the solids that were then dried in vacuo to give the title compound as a white solid.

### Intermediate 267

### N—((R)-(2-((S)-1-Amino-4,4,4-trifluoro-3,3-dimethylbutyl)-1H-benzo[d]imidazol-5-yl)(cyclopropyl)methyl)-2-(3,3-difluorocyclobutyl)acetamide

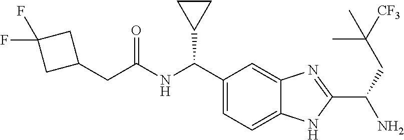 [SMILES: CC(C)(C[C@H](N)c1nc2cc([C@H](NC(=O)CC3CC(F)(F)C3)C3CC3)ccc2[nH]1)C(F)(F)F]

The title compound was prepared as described for the synthesis of Intermediate 255, using N—((R)-(2-((S)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-5-yl)(cyclopropyl)methyl)-2-(3,3-difluorocyclobutyl)acetamide (Intermediate 253) in place of N—((R)-cyclopropyl(2-((S)-1-((R)-1,1-dimethylethylsulfinamido)-4,4,4-trifluoro-3,3-dimethylbutyl)-1-((2-(trimethylsilyl)ethoxy)methyl)-1H-benzo[d]imidazol-6-yl)methyl)-2-(3,3-difluorocyclobutyl)acetamide, to provide the title compound.

### Intermediate 268

### 4,4,4-Trifluorobutanoyl Chloride

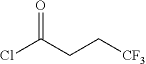

4,4,4-Trifluorobutanoic acid (5.00 g, 35.2 mmol), oxalyl dichloride (3.28 mL, 38.7 mmol), N,N-dimethylformamide (0.30 mL, 3.9 mmol) and DCM (50 mL) were added to a 100 mL round-bottomed flask equipped with mechanical stirrer, condensing tube and thermometer. The reaction mixture was stirred at rt for 2 h under a nitrogen atmosphere. The title compound was taken forward in solution without any further purification.

### Intermediate 269

### Ethyl 2-oxo-2-(2-(4,4,4-trifluorobutanoyl)hydrazinyl)acetate

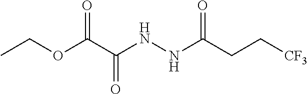 [SMILES: CCOC(=O)C(=O)NNC(=O)CCC(F)(F)F]

Ethyl 2-hydrazinyl-2-oxoacetate (4.65 g, 35.2 mmol), Et₃N (14.68 mL, 105.6 mmol) and DCM (75 mL) were added to a 250 mL round-bottomed flask equipped with mechanical stirrer, condensing tube and thermometer. The reaction mixture was stirred at rt for 10 min under a nitrogen atmosphere. Then, 4,4,4-trifluorobutanoyl chloride (5.65 g, 35.2 mmol, Intermediate 268) was added and the mixture was stirred at rt for 16 h. The reaction mixture was concentrated to dryness, then water (50 mL) was added and the mixture extracted with EtOAc (3×50 mL). The combined organic phase was dried over anhydrous Na₂SO₄, filtered, and concentrated to dryness. The crude material was purified by silica gel chromatography (33-50% EtOAc/petroleum ether) to afford the title compound as a white solid.

### Intermediate 270

### Ethyl 5-(3,3,3-trifluoropropyl)-1,3,4-oxadiazole-2-carboxylate

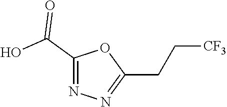 [SMILES: O=C(O)c1nnc(CCC(F)(F)F)o1]

Ethyl 2-oxo-2-(2-(4,4,4-trifluorobutanoyl)hydrazinyl)acetate (3.0 g, 12 mmol, Intermediate 269) and phosphoryl trichloride (30 mL) were added to a 50 mL single port round-bottomed flask equipped with mechanical stirrer, condensing tube and thermometer. The reaction mixture was stirred at 90° C. for 2 h. The reaction was concentrated to dryness to give a residue, to which was added water (30 mL) and basified with saturated aqueous NaHCO₃ to pH=8. Then the mixture was extracted with EtOAc (3×50 mL), the organic layers were combined, dried over anhydrous Na₂SO₄, filtered, and concentrated to dryness. The residue was purified by silica gel chromatography (20-25% EtOAc/petroleum ether) to afford the title compound as a white solid.

### Intermediate 271

### Lithium 5-(3,3,3-trifluoropropyl)-1,3,4-oxadiazole-2-carboxylate

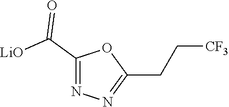

Ethyl 5-(3,3,3-trifluoropropyl)-1,3,4-oxadiazole-2-carboxylate (200 mg, 0.840 mmol, Intermediate 270), lithium hydroxide hydrate (42 mg, 1.0 mmol), MeOH (9 mL) and H₂O (3 mL) were added to a vial (40 mL) equipped with mechanical stirrer, condensing tube and thermometer. The reaction mixture was stirred at rt for 30 min under a nitrogen atmosphere. Then the reaction mixture was concentrated to dryness to provide the title compound as a white powder.

### Intermediate 272

### Cyclopropyl(1-((2-(trimethylsilyl)ethoxy)methyl)-1H-benzo[d]imidazol-5-yl)methanamine and cyclopropyl(1-((2-(trimethylsilyl)ethoxy) methyl)-1H-benzo[d]imidazol-6-yl)methanamine

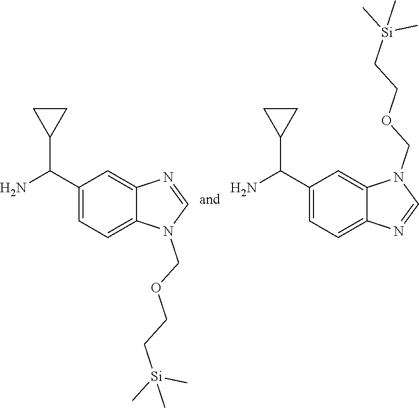 [SMILES: C[Si](C)(C)CCOCn1cnc2cc(C(N)C3CC3)ccc21 ; C[Si](C)(C)CCOCn1cnc2ccc(C(N)C3CC3)cc21]

The title compounds were prepared as described for the synthesis of Intermediate 248, using (S)—N-(cyclopropyl(1-((2-(trimethylsilyl)ethoxy)methyl)-1H-benzo[d]imidazol-5-yl)methyl)-2-methylpropane-2-sulfinamide and (S)—N-(cyclopropyl(1-((2-(trimethylsilyl)ethoxy)methyl)-1H-benzo[d]imidazol-6-yl)methyl)-2-methylpropane-2-sulfinamide (Intermediate 240) in place of (R)-cyclopropyl(1-((2-(trimethylsilyl)ethoxy)methyl)-1H-benzo[d]imidazol-5-yl)methanamine to provide the title compounds.

### Intermediate 273

### N-(Cyclopropyl(1-((2-(trimethylsilyl)ethoxy)methyl)-1H-benzo[d]imidazol-5-yl)methyl)-4,4,4-trifluorobutanamide and N-(cyclopropyl(1-((2-(trimethylsilyl)ethoxy)methyl)-1H-benzo[d]imidazol-6-yl)methyl)-4,4,4-trifluorobutanamide

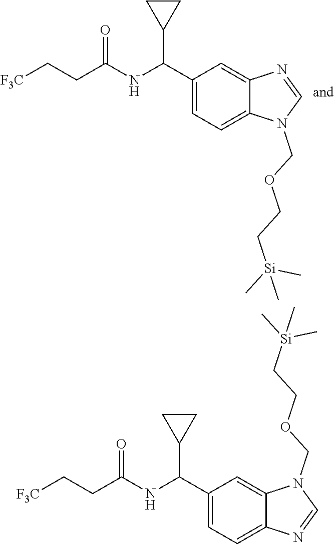

The title compounds were prepared as described for the synthesis of Intermediate 253, using cyclopropyl(1-((2-(trimethylsilyl)ethoxy)methyl)-1H-benzo[d]imidazol-5-yl)methanamine and cyclopropyl(1-((2-(trimethylsilyl)ethoxy)methyl)-1H-benzo[d]imidazol-6-yl)methanamine (Intermediate 249 in place of N—((R)-(2-((S)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-5-yl)(cyclopropyl) methyl)-2-(3,3-difluorocyclobutyl)acetamide and 4,4,4-trifluorobutanoic acid in place of 2-(3,3-difluorocyclobutyl)acetic acid, to provide the title compounds.

### Intermediate 274

### N-((2-((S*)-1-(((R)-tert-Butylsulfinyl)amino)-4,4,4-trifluoro-3,3-dimethylbutyl)-1-((2-(trimethylsilyl)ethoxy)methyl)-1H-benzo[d]imidazol-5-yl)(cyclopropyl)methyl)-4,4,4-trifluorobutanamide and N-((2-((S*)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-6-yl)(cyclopropyl)methyl)-4,4,4-trifluorobutanamide

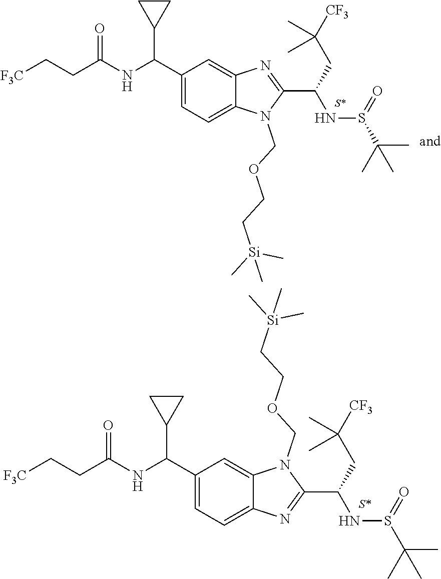

To a solution consisting of N-(cyclopropyl(1-((2-(trimethylsilyl)ethoxy)methyl)-1H-benzo[d]imidazol-5-yl)methyl)-4,4,4-trifluorobutanamide and N-(cyclopropyl(1-((2-(trimethylsilyl)ethoxy)methyl)-1H-benzo[d]imidazol-6-yl)methyl)-4,4,4-trifluorobutanamide (270 mg, 0.611 mmol, Intermediate 273) in THF (25 mL) at −78° C. was added n-BuLi (2.5 M in hexanes, 0.734 mL, 1.83 mmol) and was stirred for 3 h at −78° C. Then, (R,E)-2-methyl-N-(4,4,4-trifluoro-3,3-dimethylbutylidene)propane-2-sulfinamide (315 mg, 1.22 mmol, Intermediate 250) in THF (5 mL) was added by syringe, and the resultant mixture was allowed to warm and stir for 2 h at rt. The reaction mixture was partitioned between saturated aqueous NH₄Cl (30 mL) and EtOAc (50 mL), then the aqueous phase was extracted with EtOAc (2×30 mL). The combined organic extracts were washed with brine (50 mL), dried over anhydrous Na₂SO₄, filtered, and concentrated to dryness under reduced pressure. Purification by silica gel chromatography (0-100% EtOAc/petroleum ether) afforded the title compound as a yellow oil.

### Intermediate 275

### N-((2-((S*)-1-Amino-4,4,4-trifluoro-3,3-dimethylbutyl)-1H-benzo[d]imidazol-5-yl)(cyclopropyl)methyl)-4,4,4-trifluorobutanamide

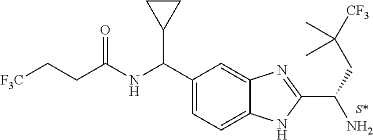 [SMILES: CC(C)(C[C@H](N)c1nc2cc(C(NC(=O)CCC(F)(F)F)C3CC3)ccc2[nH]1)C(F)(F)F]

The title compound was prepared as described for the synthesis of Intermediate 255, using N-((2-((S*)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-5-yl)(cyclopropyl)methyl)-4,4,4-trifluorobutanamide and N-((2-((S*)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-6-yl)(cyclopropyl)methyl)-4,4,4-trifluorobutanamide (Intermediate 274) in place of N—((R)-cyclopropyl(2-((S)-1-((R)-1,1-dimethylethylsulfinamido)-4,4,4-trifluoro-3,3-dimethylbutyl)-1-((2-(trimethylsilyl)ethoxy)methyl)-1H-benzo[d]imidazol-6-yl)methyl)-2-(3,3-difluorocyclobutyl)acetamide, to provide the title compound.

### Intermediate 276

### (S)—N—((R*)-1-(6-((R)-1-(1,3-Dioxoisoindolin-2-yl)ethyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide and (S)—N—((R*)-1-(5-((R)-1-(1,3-dioxoisoindolin-2-yl)ethyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide

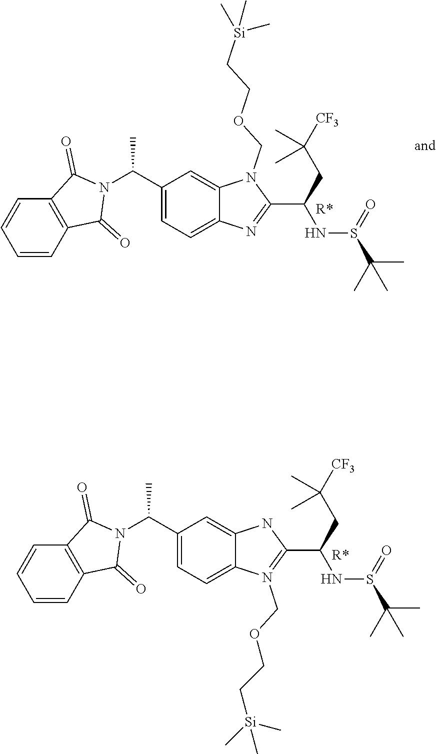

The title compounds were prepared as described for the synthesis of Intermediate 264, using (R)-2-(1-(1-((2-(trimethylsilyl)ethoxy)methyl)-1H-benzo[d]imidazol-5-yl)ethyl)isoindoline-1,3-dione and (R)-2-(1-(1-((2-(trimethylsilyl)ethoxy)methyl)-1H-benzo[d]imidazol-6-yl)ethyl)isoindoline-1,3-dione (Intermediate 263) in place of (R)-2-(cyclopropyl(1-((2-(trimethylsilyl)ethoxy)methyl)-1H-benzo[d]imidazol-5-yl)methyl)isoindoline-1,3-dione and (S,Z)-2-methyl-N-(4,4,4-trifluoro-3,3-dimethylbutylidene)propane-2-sulfinamide in place of (R,Z)-2-methyl-N-(4,4,4-trifluoro-3,3-dimethylbutylidene)propane-2-sulfinamide to provide the title compounds.

### Intermediate 277

### (S)—N—((R*)-1-(6-((R)-1-Aminoethyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide and (S)—N—((R*)-1-(5-((R)-1-aminoethyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide

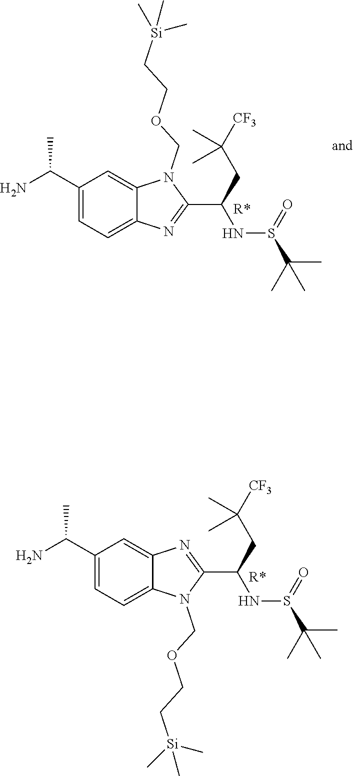 [SMILES: C[C@@H](N)c1ccc2nc([C@@H](CC(C)(C)C(F)(F)F)N[S@@](=O)C(C)(C)C)n(COCC[Si](C)(C)C)c2c1 ; C[C@@H](N)c1ccc2c(c1)nc([C@@H](CC(C)(C)C(F)(F)F)N[S@@](=O)C(C)(C)C)n2COCC[Si](C)(C)C]

The title compounds were prepared as described for the synthesis of Intermediate 265, using (S)—N—((R*)-1-(6-((R)-1-(1,3-dioxoisoindolin-2-yl)ethyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide and (S)—N—((R*)-1-(5-((R)-1-(1,3-dioxoisoindolin-2-yl)ethyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide (Intermediate 276) in place of (R)—N—((S)-1-(5-((R)-cyclopropyl(1,3-dioxoisoindolin-2-yl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide to provide the title compounds.

### Intermediate 278

### N—((R)-1-(2-((R*)-1-(((S)-tert-Butylsulfinyl)amino)-4,4,4-trifluoro-3,3-dimethylbutyl)-1-((2-(trimethylsilyl)ethoxy)methyl)-1H-benzo[d]imidazol-6-yl)ethyl)-4,4,4-trifluorobutanamide and N—((R)-1-(2-((R*)-1-(((S)-tert-butylsulfinyl)amino)-4,4,4-trifluoro-3,3-dimethylbutyl)-1-((2-(trimethylsilyl)ethoxy)methyl)-1H-benzo[d]imidazol-5-yl)ethyl)-4,4,4-trifluorobutanamide

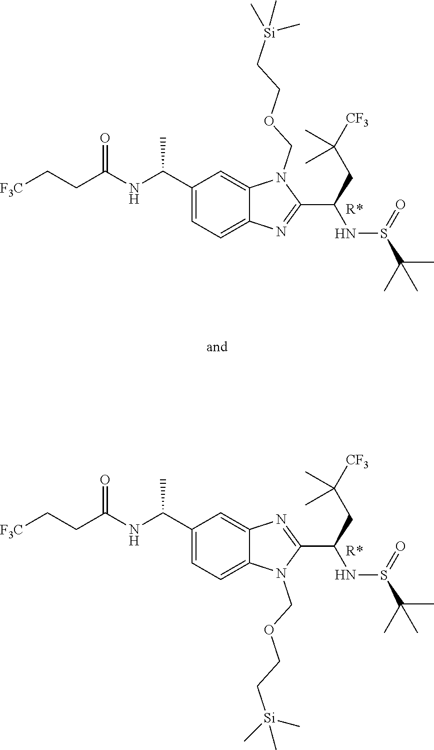 [SMILES: C[C@@H](NC(=O)CCC(F)(F)F)c1ccc2nc([C@@H](CC(C)(C)C(F)(F)F)N[S@@](=O)C(C)(C)C)n(COCC[Si](C)(C)C)c2c1 ; C[C@@H](NC(=O)CCC(F)(F)F)c1ccc2c(c1)nc([C@@H](CC(C)(C)C(F)(F)F)N[S@@](=O)C(C)(C)C)n2COCC[Si](C)(C)C]

(S)—N—((R*)-1-(6-((R)-1-Aminoethyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide and (S)—N—((R*)-1-(5-((R)-1-aminoethyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide (145 mg, 0.26 mmol, Intermediate 277) and 4,4,4-trifluorobutanoic acid (42 mg, 0.29 mmol) were combined followed by the addition of acetonitrile (4 mL), 1-methylimidazole (150 μL, 1.86 mmol) and chloro-N,N,N′,N′-tetramethylformamidinium hexafluorophosphate (99.7 mg, 0.36 mmol). The contents were stirred at rt overnight. Then, the solution was transferred to a separatory funnel where the aqueous layer was separated, salted with NaCl, then extracted three times with EtOAc. The combined organic layers were dried over magnesium sulfate, filtered, and concentrated under reduced pressure. Purification by silica gel chromatography (0-100% EtOAc/hexanes) yielded the title compound.

### Intermediate 279

### N—((R)-1-(2-((R*)-1-Amino-4,4,4-trifluoro-3,3-dimethylbutyl)-1H-benzo[d]imidazol-6-yl)ethyl)-4,4,4-trifluorobutanamide

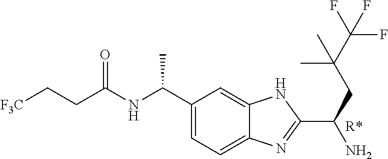 [SMILES: C[C@@H](NC(=O)CCC(F)(F)F)c1ccc2nc([C@H](N)CC(C)(C)C(F)(F)F)[nH]c2c1]

The title compound was prepared as described for the synthesis of Intermediate 255, using N—((R)-1-(2-((R*)-1-(((S)-tert-butylsulfinyl)amino)-4,4,4-trifluoro-3,3-dimethylbutyl)-1-((2-(trimethylsilyl)ethoxy)methyl)-1H-benzo[d]imidazol-6-yl)ethyl)-4,4,4-trifluorobutanamide and N—((R)-1-(2-((R*)-1-(((S)-tert-butylsulfinyl)amino)-4,4,4-trifluoro-3,3-dimethylbutyl)-1-((2-(trimethylsilyl)ethoxy)methyl)-1H-benzo[d]imidazol-5-yl)ethyl)-4,4,4-trifluorobutanamide (Intermediate 278) in place of N—((R)-cyclopropyl(2-((S)-1-((R)-1,1-dimethylethylsulfinamido)-4,4,4-trifluoro-3,3-dimethylbutyl)-1-((2-(trimethylsilyl)ethoxy)methyl)-1H-benzo[d]imidazol-6-yl)methyl)-2-(3,3-difluorocyclobutyl)acetamide.

### Intermediate 280

### N—((R)-1-(2-((S*)-1-(((R)-tert-Butylsulfinyl)amino)-4,4,4-trifluoro-3,3-dimethylbutyl)-1-((2-(trimethylsilyl)ethoxy)methyl)-1H-benzo[d]imidazol-5-yl)ethyl)-4,4,4-trifluorobutanamide and N—((R)-1-(2-((S*)-1-(((R)-tert-Butylsulfinyl)amino)-4,4,4-trifluoro-3,3-dimethylbutyl)-1-((2-(trimethylsilyl)ethoxy)methyl)-1H-benzo[d]imidazol-6-yl)ethyl)-4,4,4-trifluorobutanamide

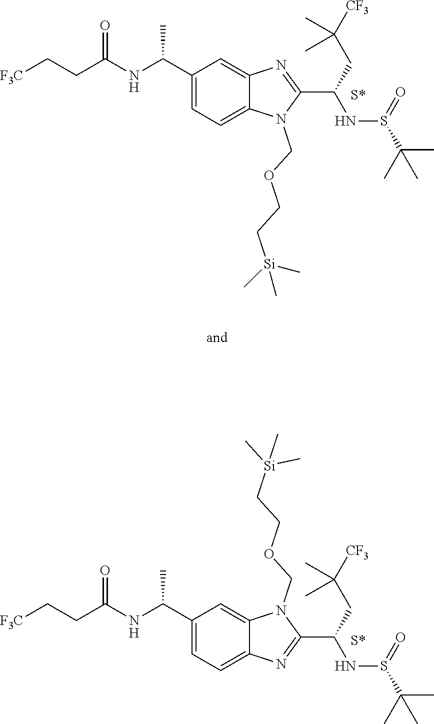 [SMILES: C[C@@H](NC(=O)CCC(F)(F)F)c1ccc2c(c1)nc([C@H](CC(C)(C)C(F)(F)F)N[S@](=O)C(C)(C)C)n2COCC[Si](C)(C)C ; C[C@@H](NC(=O)CCC(F)(F)F)c1ccc2nc([C@H](CC(C)(C)C(F)(F)F)N[S@](=O)C(C)(C)C)n(COCC[Si](C)(C)C)c2c1]

The title compounds were prepare as described for the synthesis of Intermediate 274, using (R)-4,4,4-trifluoro-N-(1-(1-((2-(trimethylsilyl)ethoxy)methyl)-1H-benzo[d]imidazol-5-yl)ethyl)butanamide and (R)-4,4,4-trifluoro-N-(1-(1-((2-(trimethylsilyl)ethoxy)methyl)-1H-benzo[d]imidazol-6-yl)ethyl)butanamide (Intermediate 147) in place of N-(cyclopropyl(1-((2-(trimethylsilyl)ethoxy)methyl)-1H-benzo[d]imidazol-5-yl)methyl)-4,4,4-trifluorobutanamide and N-(cyclopropyl(1-((2-(trimethylsilyl)ethoxy)methyl)-1H-benzo[d]imidazol-6-yl)methyl)-4,4,4-trifluorobutanamide, to provide the title compounds.

### Intermediate 281

### N—((R)-1-(2-((S*)-1-Amino-4,4,4-trifluoro-3,3-dimethylbutyl)-1H-benzo[d]imidazol-5-yl)ethyl)-4,4,4-trifluorobutanamide

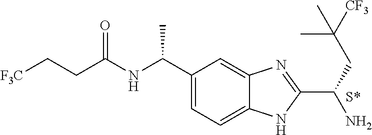 [SMILES: C[C@@H](NC(=O)CCC(F)(F)F)c1ccc2[nH]c([C@@H](N)CC(C)(C)C(F)(F)F)nc2c1]

The title compound was prepared as described for the synthesis of Intermediate 255, using N—((R)-1-(2-((S)-1-(((R)-tert-butylsulfinyl)amino)-4,4,4-trifluoro-3,3-dimethylbutyl)-1-((2-(trimethylsilyl)ethoxy)methyl)-1H-benzo[d]imidazol-5-yl)ethyl)-4,4,4-trifluorobutanamide (Intermediate 280) in place of N—((R)-cyclopropyl(2-((S)-1-((R)-1,1-dimethylethylsulfinamido)-4,4,4-trifluoro-3,3-dimethylbutyl)-1-((2-(trimethylsilyl)ethoxy)methyl)-1H-benzo[d]imidazol-6-yl)methyl)-2-(3,3-difluorocyclobutyl)acetamide, to provide the title compound.

### Intermediate 282

### (R)—N—((R)-1-(5-Bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R)-1-(6-bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide

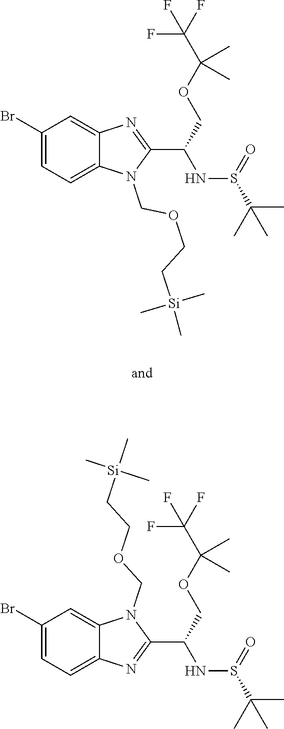 [SMILES: CC(C)(C)[S@@](=O)N[C@@H](COC(C)(C)C(F)(F)F)c1nc2cc(Br)ccc2n1COCC[Si](C)(C)C ; CC(C)(C)[S@@](=O)N[C@@H](COC(C)(C)C(F)(F)F)c1nc2ccc(Br)cc2n1COCC[Si](C)(C)C]

To a −78° C. solution of 5-bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole (4500 mg, 13.7 mmol, Intermediate 235) and (R,E)-2-methyl-N-(2-((1,1,1-trifluoro-2-methylpropan-2-yl)oxy)ethylidene)propane-2-sulfinamide (5260 mg, 19.2 mmol, Intermediate 175) in THF (140 mL) was added LDA (32 mL, 27.5 mmol, 0.85 M in THF/hexanes). The reaction was stirred at −78° C. for 30 min then quenched with AcOH (1.6 mL), warmed to rt, and poured into a mixture saturated aqueous ammonium chloride (20 mL) and brine (20 mL). The mixture was extracted with EtOAc (2×100 mL). Then the combined organic layers were washed with brine, dried over anhydrous Na₂SO₄, filtered, and condensed. Purification by silica gel chromatography (0-100% EtOAc/CH₂Cl₂) provided the title compound.

### Intermediate 283

### N—((R)-(2-((S)-Amino((1R,3s,5S)-6,6-difluorobicyclo[3.1.0]hexan-3-yl)methyl)-1H-benzo[d]imidazol-6-yl)(cyclopropyl)methyl)-2-(3,3-difluorocyclobutyl)acetamide-hydrogen chloride

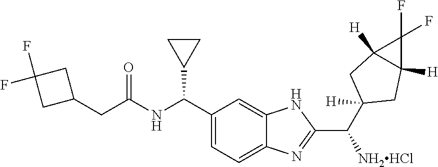

The title compound was prepared as described for the synthesis of Intermediate 361, using N—((R)-(2-((S)—(((R)-tert-butylsulfinyl)amino)((1R,3s,5S)-6,6-difluorobicyclo[3.1.0]hexan-3-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 364) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide. The reaction was directly concentrated and used crude as the HCl salt.

### Intermediate 284

### 1-Diazo-5,5,5-trifluoropentan-2-one

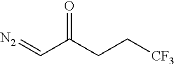

A solution of 4,4,4-trifluorobutanoyl chloride (4.5 g, 28 mmol, Intermediate 268) in MTBE (15 mL) was added dropwise to the solution of diazomethane (17 mL, 34 mmol, 2 M in n-hexane) at 0° C. over 10 min. The resulting mixture was allowed to stand for 30 min and then stirred for an additional 1 h. The title compound was used directly in the next step without purification.

### Intermediate 285

### 1-Bromo-5,5,5-trifluoropentan-2-one

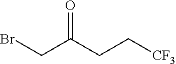

Hydrogen bromide (41.4 g, 169 mmol, 33% in AcOH) was added to the solution of 1-diazo-5,5,5-trifluoropentan-2-one (crude, ˜28 mmol in the mixture of MTBE and n-hexane, Intermediate 284). The mixture was stirred at 35° C. for 1.5 h and then concentrated to dryness to afford the title compound which was used directly in the next step without purification.

### Intermediate 286

### Ethyl 4-(3,3,3-trifluoropropyl)thiazole-2-carboxylate

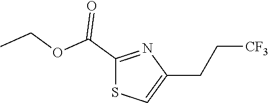 [SMILES: CCOC(=O)c1nc(CCC(F)(F)F)cs1]

1-Bromo-5,5,5-trifluoropentan-2-one (3.0 g, 13.7 mmol, Intermediate 285) was added to a mixture of ethyl 2-amino-2-thioxoacetate (1.82 g, 13.7 mmol) and anhydrous EtOH (15 mL). The resultant mixture was stirred at 75° C. for 16 h. Then the mixture was concentrated to dryness and the residue was purified by silica gel chromatography (0-9% EtOAc/petroleum ether) to afford the title compound as a colorless solid.

### Intermediate 287

### 4-(3,3,3-Trifluoropropyl)thiazole-2-carboxylic acid

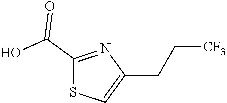 [SMILES: O=C(O)c1nc(CCC(F)(F)F)cs1]

Lithium hydroxide hydrate (2.12 g, 50.5 mmol) was added to a solution of ethyl 4-(3,3,3-trifluoropropyl)thiazole-2-carboxylate (2.56 g, 10.1 mmol, Intermediate 286) in THF (30 mL) and H₂O (6 mL). After addition, the reaction mixture was stirred at rt for 3 h. The reaction mixture was acidified by 1 N aqueous HCl to pH=4, and then the aqueous phase was extracted with EtOAc (5×30 mL). The organic phases were combined, dried over anhydrous Na₂SO₄, filtered and concentrated to dryness. The residue was purified by preparative HPLC (column: C18 spherical, 20-35 μm, 80 g, 5-40% (v/v) water-ACN) to afford the title compound as a white solid.

### Intermediate 288

### (EZ)-4,4,4-Trifluorobutanal Oxime

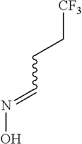

Potassium carbonate (3.29 g, 23.8 mmol) was added to a mixture of 4,4,4-trifluorobutanal (2 g, 15.9 mmol), hydroxylamine hydrochloride (1.21 g, 17.5 mmol) and EtOH (20 mL) and the resulting mixture was stirred at rt for 16 h. After that time, the mixture was concentrated to dryness, diluted with water (10 mL) and extracted with DCM (3×10 mL). The organic layers were combined, washed with brine (20 mL), dried over anhydrous Na₂SO₄, filtered, and concentrated to dryness to provide the title compound as a colorless oil.

### Intermediate 289

### Methyl 3-(3,3,3-trifluoropropyl)isoxazole-4-carboxylate and methyl 3-(3,3,3-trifluoroproyl)isoxazole-5-carboxylate

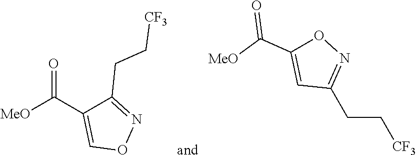 [SMILES: COC(=O)c1conc1CCC(F)(F)F ; "and" ; COC(=O)c1cc(CCC(F)(F)F)no1]

NCS (852 mg, 6.38 mmol) and NaHCO₃ (376 mg, 4.48 mmol) were added to a solution of 4,4,4-trifluorobutanal oxime (600 mg, 4.25 mmol, Intermediate 288) and methyl propiolate (358 mg, 4.26 mmol) in chloroform (3 mL). The reaction mixture was stirred at 65° C. for 16 h. The mixture was concentrated under reduced pressure, and the residue was dispersed into EtOAc (20 mL), then washed with water (10 mL) and brine (10 mL), dried over anhydrous Na₂SO₄, filtered through silica gel, and concentrated to dryness. The residue was purified by silica gel chromatography (9-100% EtOAc/petroleum ether) to provide a mixture of the title compounds as a colorless oil.

### Intermediate 290

### 3-(3,3,3-Trifluoropropyl)isoxazole-4-carboxylic acid and 3-(3,3,3-trifluoropropyl)isoxazole-5-carboxylic acid

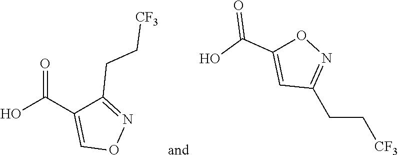 [SMILES: O=C(O)c1conc1CCC(F)(F)F ; "and" ; O=C(O)c1cc(CCC(F)(F)F)no1]

Sodium hydroxide (3.6 mL, 7.2 mmol, 2 M in H₂O) was added to a mixture of methyl 3-(3,3,3-trifluoropropyl)isoxazole-4-carboxylate and methyl 3-(3,3,3-trifluoropropyl)isoxazole-5-carboxylate (400 mg, 0.9 mmol, Intermediate 289) and EtOH (3.5 mL). The resultant mixture was stirred at rt for 2 h, then concentrated to dryness and the residue was diluted with water (5 mL). The resultant mixture was acidified with 1 N aqueous HCl to pH 4 and extracted with EtOAc (3×20 mL). The organic layers were combined, washed with brine (10 mL), dried over anhydrous Na₂SO₄, filtered, and concentrated to dryness to provide a mixture of the title compounds as a white solid.

### Intermediate 291

### Methyl 1-(3,3,3-trifluoropropyl)-1H-imidazole-5-carboxylate

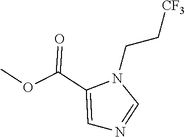

Methyl 1H-imidazole-4-carboxylate (2.0 g, 15.9 mmol), 1,1,1-trifluoro-3-iodopropane (7.1 g, 31.7 mmol), cesium carbonate (10.3 g, 31.6 mmol) and ACN (30 mL) were added to an autoclave. The resultant mixture was stirred at 80° C. for 16 h. After that time, the mixture was cooled to rt and filtered. The filtrate was concentrated to dryness, suspended in H₂O (30 mL) and extracted with EtOAc (3×60 mL). The organic layers were combined, washed with brine (30 mL), dried over anhydrous Na₂SO₄, filtered and concentrated to dryness. The residue was purified by preparative HPLC (Xtimate C18, 150×30 mm, 5 m column (20-50% (v/v) CH₃CN in H₂O with 0.05% NH₄OH)) to provide the title compound, the second eluting isomer, as a colorless solid.

### Intermediate 292

### 1-(3,3,3-Trifluoropropyl)-1H-imidazole-5-carboxylic acid

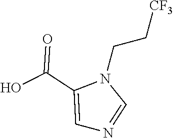 [SMILES: O=C(O)c1cncn1CCC(F)(F)F]

The title compound was prepared as described for the synthesis of Intermediate 290, using methyl 1-(3,3,3-trifluoropropyl)-1H-imidazole-5-carboxylate (Intermediate 291) in place of a mixture of methyl 3-(3,3,3-trifluoropropyl)isoxazole-4-carboxylate and methyl 3-(3,3,3-trifluoropropyl)isoxazole-5-carboxylate. The reaction was run in MeOH instead of EtOH, and additional NaOH (0.42 mL, 0.84 mmol, 2 M in water) and MeOH (3 mL) were added after stirring at rt for 2 h, and the mixture was stirred for an additional 16 h at rt. The reaction mixture was acidified to pH 6 instead of pH 4, and after extraction with EtOAc, it was this aqueous layer that was frozen and lyophilized to provide the title compound as a white solid.

### Intermediate 293

### Ethyl 5-(3,3,3-trifluoropropyl)thiophene-2-carboxylate

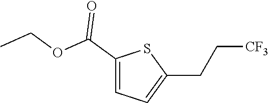 [SMILES: CCOC(=O)c1ccc(CCC(F)(F)F)s1]

1,2-Dibromoethane (0.269 mL, 3.57 mmol) was added to a vigorously stirred solution of zinc (2.19 g, 33.5 mmol) in THF (20 mL) under a nitrogen atmosphere. The suspension was stirred at 80° C. for 10 min before chlorotrimethylsilane (0.425 mL, 3.35 mmol) was added at rt. The mixture was stirred at 40° C. for 30 min, then a solution of 1,1,1-trifluoro-3-iodopropane (5.00 g, 22.3 mmol) was added dropwise to the solution over a period of 10 min. The reaction mixture was stirred at rt for 16 h, and then was used directly in the next step. Ethyl 5-bromothiophene-2-carboxylate (600 mg, 2.55 mmol) was added to the above mixture, and the reactor was backfilled with N2 three times before bis(tri-tert-butylphosphine)palladium (143 mg, 0.28 mmol) was added. The resultant mixture was stirred at 55° C. for 16 h before cooling to rt. After this time, the mixture was filtered and the filtrate was concentrated to dryness to give the crude product, which was purified by reverse-phase silica gel chromatography (spherical C18; 30-60% ACN/water) to provide the title compound as a white solid.

### Intermediate 294

### 5-(3,3,3-Trifluoropropyl)thiophene-2-carboxylic acid

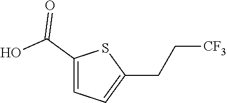 [SMILES: O=C(O)c1ccc(CCC(F)(F)F)s1]

Aqueous NaOH (0.6 mL, 1.2 mmol, 2 M) was added to a solution of ethyl 5-(3,3,3-trifluoropropyl)thiophene-2-carboxylate (70 mg, 0.28 mmol, Intermediate 293) in EtOH (4 mL) and the resultant mixture was stirred at rt for 3 h. After this time, EtOH was removed under reduced pressure and the residue was diluted with water (5 mL). The mixture was acidified with 1 N aqueous HCl to pH=4 and extracted with EtOAc (3×20 mL). The combined organic extracts were washed with brine, dried over anhydrous Na₂SO₄, filtered and concentrated to dryness to afford the title compound as a white solid.

### Intermediate 295

### 4,4,4-Trifluorobutanethioamide

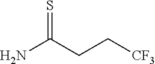

Lawesson's reagent (4.6 g, 11 mmol) was added to a solution consisting of 4,4,4-trifluorobutanamide (1.6 g, 11 mmol) and toluene (45 mL). The resultant mixture was stirred for at 80° C. 3 h. After this time, the mixture was filtered to afford a solution of the crude title compound, which was used in the next step without purification.

### Intermediate 296

### Ethyl 2-(3,3,3-trifluoropropyl)thiazole-4-carboxylate

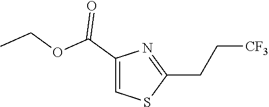 [SMILES: CCOC(=O)c1csc(CCC(F)(F)F)n1]

CaCO₃ (130 mg, 1.30 mmol) was added in portions to a solution consisting of 4,4,4-trifluorobutanethioamide (400 mg, 2.55 mmol, crude in 10 mL toluene, Intermediate 295), ethyl 3-bromo-2-oxopropanoate (350 mL, 2.81 mmol) and EtOH (2 mL). The resultant mixture was stirred at rt for 16 h. After this time, the mixture was concentrated to dryness to afford a yellow oil, which was diluted with EtOAc (10 mL), washed with brine (10 mL), dried over anhydrous Na₂SO₄, filtrated and concentrated to dryness to afford the crude title compound as a yellow oil, which was used in the next step without purification.

### Intermediate 297

### 2-(3,3,3-Trifluoropropyl)thiazole-4-carboxylic acid

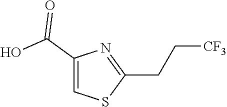 [SMILES: O=C(O)c1csc(CCC(F)(F)F)n1]

NaOH (0.80 mL, 1.6 mmol, 2 M in H₂O) was added to a solution of ethyl 2-(3,3,3-trifluoropropyl)thiazole-4-carboxylate (200 mg, 0.79 mmol, Intermediate 296) in MeOH (4 mL). The resultant mixture was stirred at rt for 2 h. After this time, the mixture was concentrated to dryness and the residue was dissolved in H₂O (5 mL), acidified with 1 N aqueous HCl to pH=2, and extracted with EtOAc (3×10 mL). The combined organic extracts were washed with brine, dried over anhydrous Na₂SO₄, filtered, and concentrated to dryness. The residue was purified by silica gel chromatography using a spherical C18, 20-35 μm column (5-50% (v/v) CH₃CN in H₂O), and the product fractions were suspended in water (15 mL), frozen using dry ice/ethanol, and then lyophilized to dryness to afford the title compound as a colorless solid.

### Intermediate 298

### Methyl 2-(3,3,3-trifluoropropyl)-4,5-dihydrooxazole-4-carboxylate

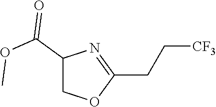

DABCO (8.65 g, 77.1 mmol) was added to a suspension of L-serine methyl ester hydrochloride (4.00 g, 25.7 mmol) in DCM (100 mL). The resultant mixture was stirred at rt for 20 min, then treated with 4,4,4-trifluorobutanal (3.24 g, 25.7 mmol) and stirred at rt for 30 min. Then the reaction mixture was cooled to 0° C., treated with NCS (3.43 g, 25.7 mmol), and stirred for 16 h while gradually warming to rt. After this time, the mixture was quenched with saturated aqueous Na₂S₂O₅ (100 mL) and extracted with EtOAc (2×100 mL). The combined organic extracts were washed with saturated aqueous NaHCO₃ (50 mL) and brine (50 mL), dried over anhydrous Na₂SO₄, and concentrated to dryness. The residue was purified by silica gel chromatography (9-25% EtOAc/petroleum ether) to provide the title compound as a colorless oil.

### Intermediate 299

### Methyl 2-(3,3,3-trifluoropropyl)oxazole-4-carboxylate

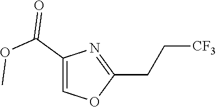

NBS (474 mg, 2.66 mmol) was added to a suspension of methyl 2-(3,3,3-trifluoropropyl)-4,5-dihydrooxazole-4-carboxylate (500 mg, 2.22 mmol, Intermediate 298), K₂CO₃ (368 mg, 2.66 mmol) and 4 Å MS (1.0 g) in DCM (10 mL) and the resultant mixture was stirred at 45° C. for 16 h. After this time, the mixture was cooled to 0° C. and filtered. The filtrate was treated with saturated aqueous Na₂S₂O₃ (10 mL) followed by saturated aqueous NaHCO₃ (10 mL). The resultant mixture was extracted with DCM (2×25 mL). The combined organic extracts were washed with saturated aqueous NaHCO₃ (10 mL) and brine (10 mL), dried over anhydrous Na₂SO₄, filtered, and concentrated to dryness. The crude residue was purified by preparative HPLC (Boston Prime C18, 150×30 mm, 5 m column, (25-55% (v/v) CH₃CN in H₂O with 0.04% NH₄OH and 10 mM NH₄HCO₃)) to provide the title compound as a colorless oil.

### Intermediate 300

### 2-(3,3,3-Trifluoropropyl)oxazole-4-carboxylic acid

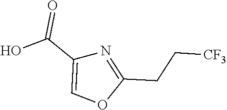 [SMILES: O=C(O)c1coc(CCC(F)(F)F)n1]

NaOH (1.2 mL, 2.4 mmol, 2 M in H₂O) was added dropwise to a solution of methyl 2-(3,3,3-trifluoropropyl)oxazole-4-carboxylate (120 mg, 0.54 mmol, Intermediate 299) in methanol (6 mL) and the resultant mixture was stirred at rt for 3 h. After this time, the mixture was concentrated to dryness. The residue was dissolved with water (5 mL), acidified with 1 N aqueous HCl to pH 4, and extracted with EtOAc (3×20 mL). The organic layers were combined, washed with brine (10 mL), dried over anhydrous Na₂SO₄, filtered, and concentrated to dryness to provide the title compound as a white solid.

### Intermediate 301

### Methyl 1-(3,3,3-trifluoropropyl)-1H-imidazole-4-carboxylate

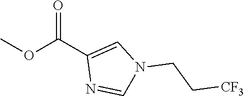

The title compound was prepared as described for the synthesis of Intermediate 291. Methyl 1-(3,3,3-trifluoropropyl)-1H-imidazole-4-carboxylate was the first eluting isomer, isolated as a white solid.

### Intermediate 302

### 1-(3,3,3-Trifluoropropyl)-1H-imidazole-4-carboxylic acid

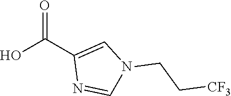 [SMILES: O=C(O)c1cn(CCC(F)(F)F)cn1]

Sodium hydroxide (0.8 mL, 1.6 mmol, 2 M in H₂O) was added to a mixture of methyl 1-(3,3,3-trifluoropropyl)-1H-imidazole-4-carboxylate (178 mg, 0.8 mmol, Intermediate 301) and MeOH (3 mL). The resultant mixture was stirred at rt for 16 h, then concentrated to dryness. The residue was purified by reverse-phase silica gel chromatography (spherical C18; 5-100% ACN/water) to provide the title compound as a colorless solid.

### Intermediate 303

### Methyl 1-(3,3,3-trifluoropropyl)-1H-imidazole-2-carboxylate

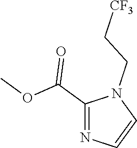

To a mixture of methyl 1H-imidazole-2-carboxylate (500 mg, 3.97 mmol) in MeCN (4 mL) was added 1,1,1-trifluoro-3-iodopropane (0.9 mL, 8.1 mmol) and cesium carbonate (2.6 g, 8.0 mmol). The resultant mixture was stirred at 60° C. for 18 h. After that time, the mixture was cooled to rt and filtered. The filtrate was concentrated to dryness to give the crude product, which was purified by silica gel chromatography (0-80% EtOAc/petroleum ether) to afford the title compound as a white solid.

### Intermediate 304

### 1-(3,3,3-Trifluoropropyl)-1H-imidazole-2-carboxylic acid

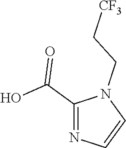 [SMILES: O=C(O)c1nccn1CCC(F)(F)F]

Aqueous NaOH (0.6 mL, 1.2 mmol, 2 M) was added to a solution of methyl 1-(3,3,3-trifluoropropyl)-1H-imidazole-2-carboxylate (70 mg, 0.28 mmol, Intermediate 303) in EtOH (4 mL) and the resultant mixture was stirred at rt for 3 h. After this time, EtOH was removed under reduced pressure and the residue was diluted with water (5 mL). The mixture was acidified with 1 N aqueous HCl to pH=4 and extracted with EtOAc (3×20 mL). The combined organic extracts were washed with brine, dried over anhydrous Na₂SO₄, filtered and concentrated to dryness to afford the title compound as a white solid.

### Intermediate 305

### Methyl 1-isopropyl-1H-1,2,4-triazole-5-carboxylate

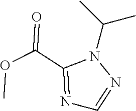

To a microwave vial was added methyl-1H-1,2,4-triazole-3-carboxylate (500 mg, 3.93 mmol), propan-2-ol (473 mg, 7.87 mmol), tricyclohexylphosphine (1.2 g, 4.3 mmol), DBAD (1.4 g, 6.08 mmol) and THF (8 mL). The resulting mixture was stirred at 110° C. in the microwave for 1.5 h. The mixture was then diluted with water (50 mL) and extracted with EtOAc (2×50 mL). The organic layers were combined, dried over anhydrous Na₂SO₄, filtered, and concentrated to dryness. The residue was purified by silica gel chromatography (0-15% EtOAc/petroleum ether) to provide the title compound as a white solid.

### Intermediate 306

### Methyl 1-(3,3,3-trifluoropropyl)-1H-1,2,4-triazole-5-carboxylate

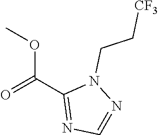

The title compound was prepared as described for the synthesis of Intermediate 15, using methyl 1H-1,2,4-triazole-3-carboxylate in place of ethyl 1H-1,2,3-triazole-4-carboxylate and 3,3,3-trifluoropropan-1-ol in place of 2-cyclopropylethanol, and adding the DIAD to a rt mixture and stirring at 120° C. in the microwave for 2 h instead of rt, to provide the title compound as a clear colorless oil.

### Intermediate 307

### Methyl 1-(3,3,3-trifluoropropyl)-1H-1,2,4-triazole-3-carboxylate

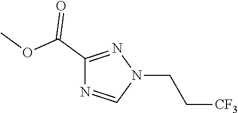

A mixture of methyl 1H-1,2,3-triazole-4-carboxylate (3 g, 23.6 mmol) in DMF (29.5 mL) was cooled to 0° C. and then NaH (1.89 g, 47.2 mmol, 60% dispersion in mineral oil) was added and the mixture stirred at rt for 5 min. The reaction mixture was then cooled to 0° C. and 1,1,1-trifluoro-3-iodopropane (4.15 mL, 35.4 mmol) was added dropwise. The resulting mixture was stirred for 1 h while gradually warming to rt, and then stirred at rt for an additional 16 h. The mixture was then poured into ice water (30 mL) and extracted with EtOAc (3×50 mL). The organic layers were combined, washed with brine, dried over anhydrous Na₂SO₄, filtered, and concentrated to dryness. The residue was purified by silica gel chromatography (0-100% EtOAc/hexanes) to provide the title compound as a yellow solid.

### Intermediate 308

### 1-(3,3,3-Trifluoropropyl)-1H-1,2,4-triazole-3-carboxylic acid

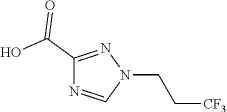 [SMILES: O=C(O)c1ncn(CCC(F)(F)F)n1]

The title compound was prepared as described for the synthesis of Intermediate 7, using methyl 1-(3,3,3-trifluoropropyl)-1H-1,2,4-triazole-3-carboxylate (Intermediate 307) in place of methyl 2-(3,3,3-trifluoropropyl)-2H-1,2,3-triazole-4-carboxylate. The aqueous layer was acidified to ˜pH 1-2, and the product precipitated out of solution. The solids were isolated by filtration, rinsing with water, and dried to provide the title compound as a white solid.

### Intermediate 309

### (S)—N—((S)-1-(6-Bromo-1-((2-(trimethylsilyl)ethoxy)methyl)-1H-benzo[d]imidazol-2-yl)-4,4,4-trifluoro-3,3-dimethylbutyl)-2-methylpropane-2-sulfinamide and (S)—N—((S)-1-(5-bromo-1-((2-(trimethylsilyl)ethoxy)methyl)-1H-benzo[d]imidazol-2-yl)-4,4,4-trifluoro-3,3-dimethylbutyl)-2-methylpropane-2-sulfinamide

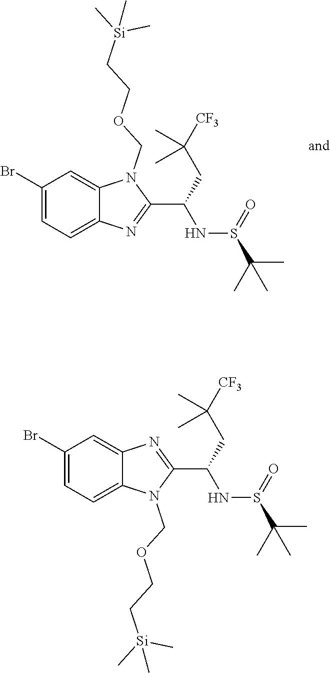 [SMILES: CC(C)(C)[S@](=O)N[C@@H](CC(C)(C)C(F)(F)F)c1nc2ccc(Br)cc2n1COCC[Si](C)(C)C ; CC(C)(C)[S@](=O)N[C@@H](CC(C)(C)C(F)(F)F)c1nc2cc(Br)ccc2n1COCC[Si](C)(C)C]

The title compounds were prepared as described for the synthesis of Intermediate 251, using 5-bromo-1-((2-(trimethylsilyl)ethoxy)methyl)-1H-benzo[d]imidazole and 6-bromo-1-((2-(trimethylsilyl)ethoxy)methyl)-1H-benzo[d]imidazole (Intermediate 235) in place of (R)-cyclopropyl(1-((2-(trimethylsilyl)ethoxy)methyl)-1H-benzo[d]imidazol-5-yl)methanamine and LDA in place of n-BuLi, to provide the mixture of title compounds.

### Intermediate 310

### (R)—N—((S)-1-(5-Bromo-1H-benzo[d]imidazol-2-yl)-4,4,4-trifluoro-3,3-dimethylbutyl)-2-methylpropane-2-sulfinamide

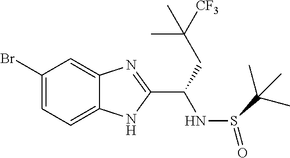 [SMILES: CC(C)(C)[S@@](=O)N[C@@H](CC(C)(C)C(F)(F)F)c1nc2cc(Br)ccc2[nH]1]

To (S)—N—((S)-1-(6-bromo-1-((2-(trimethylsilyl)ethoxy)methyl)-1H-benzo[d]imidazol-2-yl)-4,4,4-trifluoro-3,3-dimethylbutyl)-2-methylpropane-2-sulfinamide and (S)—N—((S)-1-(5-bromo-1-((2-(trimethylsilyl)ethoxy)methyl)-1H-benzo[d]imidazol-2-yl)-4,4,4-trifluoro-3,3-dimethylbutyl)-2-methylpropane-2-sulfinamide (45.0 g, 76.9 mmol, Intermediate 309) was added TBAF (384 mL, 1 M in THF) and the mixture was stirred for 12 h at 90° C. The reaction mixture was concentrated under reduced pressure then purified by silica gel chromatography (9-50% EtOAc/petroleum ether) to provide the title compound.

### Intermediate 311

### (R)—N—((S)-1-(5-Cyano-1H-benzo[d]imidazol-2-yl)-4,4,4-trifluoro-3,3-dimethylbutyl)-2-methylpropane-2-sulfinamide

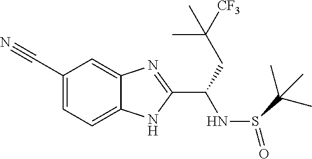 [SMILES: CC(C)(C)[S@@](=O)N[C@@H](CC(C)(C)C(F)(F)F)c1nc2cc(C#N)ccc2[nH]1]

Zn(CN)₂ (0.730 g, 6.22 mmol) was added to a solution consisting of (R)—N—((S)-1-(5-bromo-1H-benzo[d]imidazol-2-yl)-4,4,4-trifluoro-3,3-dimethylbutyl)-2-methylpropane-2-sulfinamide (2.50 g, 5.50 mmol, Intermediate 310) and a mixture of 1,4-dioxane-water (3:1, 150 mL). The resultant mixture was sparged with nitrogen for 5 min and then treated with XPhos (1.02 g, 2.14 mmol) and Pd₂(dba)₃ (1.0 g, 1.09 mmol). The resultant mixture was sparged with nitrogen for another 5 min and then stirred at 100° C. for 16 h. The contents were then cooled to rt and filtered. The filtrate was concentrated to dryness under reduced pressure to afford the title compound which was directly used to the next step without any purification.

### Intermediate 312

### (S)-2-(1-Amino-4,4,4-trifluoro-3,3-dimethylbutyl)-1H-benzo[d]imidazole-5-carbonitrile

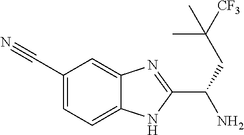 [SMILES: CC(C)(C[C@H](N)c1nc2cc(C#N)ccc2[nH]1)C(F)(F)F]

The title compound was prepared as described for the synthesis of Intermediate 255, using (R)—N—((S)-1-(5-cyano-1H-benzo[d]imidazol-2-yl)-4,4,4-trifluoro-3,3-dimethylbutyl)-2-methylpropane-2-sulfinamide (Intermediate 311) in place of N—((R)-cyclopropyl(2-((S)-1-((R)-1,1-dimethylethylsulfinamido)-4,4,4-trifluoro-3,3-dimethylbutyl)-1-((2-(trimethylsilyl)ethoxy)methyl)-1H-benzo[d]imidazol-6-yl)methyl)-2-(3,3-difluorocyclobutyl)acetamide, to provide the title compound.

### Intermediate 313

### (S)—N-(1-(5-Cyano-1H-benzo[d]imidazol-2-yl)-4,4,4-trifluoro-3,3-dimethylbutyl)-1-(3,3,3-trifluoropropyl)-1H-pyrazole-5-carboxamide

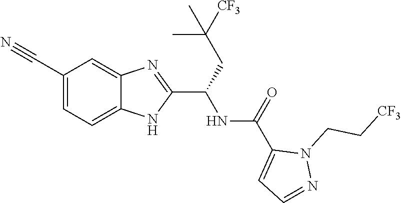 [SMILES: CC(C)(C[C@H](NC(=O)c1ccnn1CCC(F)(F)F)c1nc2cc(C#N)ccc2[nH]1)C(F)(F)F]

To a solution of 1-(3,3,3-trifluoropropyl)-1H-pyrazole-5-carboxylic acid (670 mg 3.22 mmol) and HOAt (530 mg, 3.89 mmol) in DCM (30 mL) at 0° C. was added EDCI (570 mg, 2.97 mmol), and the resulting mixture was warmed to rt over 30 min. Next, (S)-2-(1-amino-4,4,4-trifluoro-3,3-dimethylbutyl)-1H-benzo[d]imidazole-5-carbonitrile (1 g, 2.7 mmol, Intermediate 312) and DIPEA (2.1 mL, 11.96 mmol) were added and the mixture was stirred at rt for 1 h. The crude reaction mixture was diluted with water (50 mL) and extracted with EtOAc (3×50 mL). The combined organic layers were washed with brine, dried over anhydrous Na₂SO₄, filtered, and concentrated under reduced pressure. Purification by silica gel chromatography (9-33% EtOAc/petroleum ether) yielded the title compound as a light-yellow solid.

### Intermediate 314

### N-((1S)-1-(5-(1-Aminoethyl)-1H-benzo[d]imidazol-2-yl)-4,4,4-trifluoro-3,3-dimethylbutyl)-1-(3,3,3-trifluoropropyl)-1H-pyrazole-5-carboxamide

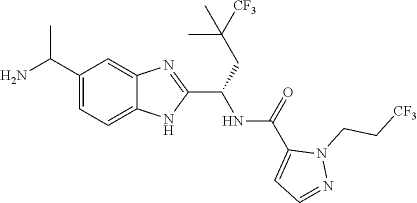 [SMILES: CC(N)c1ccc2[nH]c([C@H](CC(C)(C)C(F)(F)F)NC(=O)c3ccnn3CCC(F)(F)F)nc2c1]

Methylmagnesium bromide (3.00 mL, 9.00 mmol, 3 M in Et₂O) was added to a mixture of (S)—N-(1-(5-cyano-1H-benzo[d]imidazol-2-yl)-4,4,4-trifluoro-3,3-dimethylbutyl)-1-(3,3,3-trifluoropropyl)-1H-pyrazole-5-carboxamide (400 mg, 0.822 mmol, Intermediate 313) and copper(I) iodide (93.6 mg, 0.489 mmol) in THF (15 mL) under Ar. The reaction mixture was stirred for 30 min at 100° C. in a microwave. The reaction mixture was added to a solution of NaBH₄ (310 mg, 8.19 mmol) in MeOH (15 mL) and stirred for 1 h at rt. The mixture was filtered through a pad of Celite®. The solution was adjusted to pH=3 using 1 N aqueous HCl and then adjusted to pH=10 with 1 N aqueous NaOH. The mixture was extracted with EtOAc (3×20 mL), and then the combined organic layers were washed with brine, dried over anhydrous Na₂SO₄, filtered, and concentrated to dryness under reduced pressure to afford the title compound which was directly used in the next step without any additional purification.

### Intermediate 315

### (S)—N-(1-(5-Cyano-1H-benzo[d]imidazol-2-yl)-4,4,4-trifluoro-3,3-dimethylbutyl)-1-isopropyl-1H-pyrazole-5-carboxamide

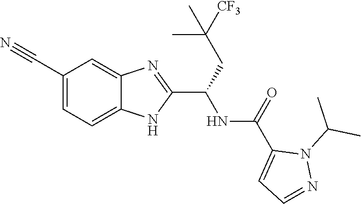 [SMILES: CC(C)n1nccc1C(=O)N[C@@H](CC(C)(C)C(F)(F)F)c1nc2cc(C#N)ccc2[nH]1]

The title compound was prepared as described for the synthesis of Intermediate 313, using 1-isopropyl-1H-pyrazole-5-carboxylic acid in place of 1-(3,3,3-trifluoropropyl)-1H-pyrazole-5-carboxylic acid and stirring for 2 h in place of 1 h, to provide the title compound as a light-yellow solid.

### Intermediate 316

### N-((15)-1-(5-(1-Aminoethyl)-1H-benzo[d]imidazol-2-yl)-4,4,4-trifluoro-3,3-dimethylbutyl)-1-isopropyl-1H-pyrazole-5-carboxamide

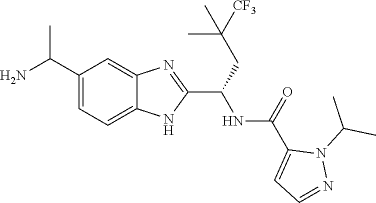 [SMILES: CC(N)c1ccc2[nH]c([C@H](CC(C)(C)C(F)(F)F)NC(=O)c3ccnn3C(C)C)nc2c1]

The title compound was prepared as described for the synthesis of Intermediate 314, using (S)—N-(1-(5-cyano-1H-benzo[d]imidazol-2-yl)-4,4,4-trifluoro-3,3-dimethylbutyl)-1-isopropyl-1H-pyrazole-5-carboxamide (Intermediate 315) in place of (S)—N-(1-(5-cyano-1H-benzo[d]imidazol-2-yl)-4,4,4-trifluoro-3,3-dimethylbutyl)-1-(3,3,3-trifluoropropyl)-1H-pyrazole-5-carboxamide to provide the title compound.

### Intermediate 317

### 4-Methoxybenzyl 3-(bromomethylene)cyclobutane-1-carboxylate

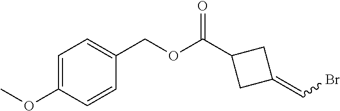 [SMILES: COc1ccc(COC(=O)C2CC(=CBr)C2)cc1]

(Bromomethyl)triphenylphosphonium bromide (39 g, 90 mmol) and THF (150 mL) were added to a 250 mL three-necked round-bottomed flask equipped with a mechanical stirrer, condensing tube, and thermometer. A solution of t-BuOK in THF (1 M, 85 mL, 85 mmol) was added at −78° C. under N2. The resulting mixture was stirred at −78° C. for 1 h. 4-Methoxybenzyl 3-oxocyclobutanecarboxylate (10 g, 43 mmol) was added and the reaction mixture was stirred for 1 h at −78° C., followed by an additional 2 h at 0° C. The reaction was then concentrated to dryness under reduced pressure to give the crude product. Water (200 mL) was then added and extracted with EtOAc (3×50 mL). The combined organic extracts were washed with brine (100 mL), dried over anhydrous Na₂SO₄, filtered, and concentrated to dryness under reduced pressure to give the crude product, which was purified by silica gel chromatography (10-20% EtOAc/petroleum ether) to afford the title compound as a yellow oil.

### Intermediate 318

### 4-Methoxybenzyl 3-(2,2,2-trifluoroethylidene)cyclobutane-1-carboxylate

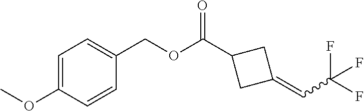 [SMILES: COc1ccc(COC(=O)C2CC(=CC(F)(F)F)C2)cc1]

Methyl fluorosulfonyldifluoroacetate (7.4 g, 39 mmol) in anhydrous DMF (10 mL) was added dropwise via syringe to a suspension of 4-methoxybenzyl 3-(bromomethylene) cyclobutanecarboxylate (4.0 g, 13 mmol, Intermediate 317) and CuI (3.7 g, 19 mmol) in anhydrous DMF (40 mL) and HMPA (20 mL) at 75° C. under argon over a period of 1 h, and the resulting suspension was stirred at rt under argon for 6 days. After this time, the reaction was then concentrated to dryness under reduced pressure to give the crude product. The crude product was diluted with water (20 mL) and extracted with EtOAc (3×20 mL). The combined organic extracts were washed with brine (30 mL), dried over anhydrous Na₂SO₄, filtered, and concentrated to dryness under reduced pressure to give the crude product. The crude material was purified by silica gel chromatography (0-10% EtOAc/petroleum ether) to give the title compound as a yellow oil.

### Intermediate 319

### 4-Methoxybenzyl 3-(2,2,2-trifluoroethyl)cyclobutane-1-carboxylate

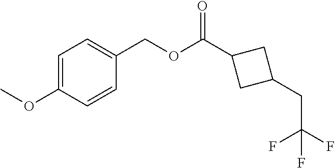 [SMILES: COc1ccc(COC(=O)C2CC(CC(F)(F)F)C2)cc1]

4-Methoxybenzyl 3-(2,2,2-trifluoroethylidene)cyclobutanecarboxylate (7.0 g, 23 mmol, Intermediate 318), MeOH (150 mL), and dry Pd/C (3.0 g, 10% Pd) were added to a 250 mL hydrogenation bottle. The resultant mixture was stirred under H₂ (50 psi) at rt for 16 h. After this time, the suspension was filtered through a pad of Celite® and was washed with MeOH (100 mL). The filtrate was concentrated to dryness under reduced pressure to afford the title compound as a colorless oil that was used without further purification.

### Intermediate 320

### 3-(2,2,2-Trifluoroethyl)cyclobutane-1-carboxylic acid

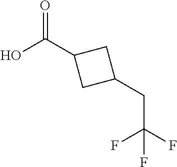 [SMILES: O=C(O)C1CC(CC(F)(F)F)C1]

4-Methoxybenzyl 3-(2,2,2-trifluoroethyl)cyclobutanecarboxylate (7.0 g, 23 mmol, Intermediate 319), LiOH·H₂O (15 g, 35 mmol), THF (20 mL), and H₂O (20 mL) were added to a 100 mL one-necked round-bottomed flask equipped with mechanical stirrer, condensing tube, and thermometer. The resultant solution was allowed to stir at rt for 16 h. After this this time, the reaction was concentrated to remove the THF. The solution was adjusted with 1 M aqueous HCl to pH ˜3-4. The product was then diluted with water (100 mL) and extracted with EtOAc (3×30 mL). The combined organic extracts were washed with brine (50 mL), dried over anhydrous Na₂SO₄, filtered, and concentrated to dryness under reduced pressure to give the crude product. The crude material was purified by silica gel chromatography (0-20% EtOAc/petroleum ether) to give the title compound as a colorless oil.

### Intermediate 321

### N-Methoxy-N-methyl-3-(2,2,2-trifluoroethyl)cyclobutane-1-carboxamide

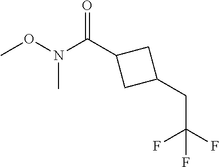

3-(2,2,2-Trifluoroethyl)cyclobutanecarboxylic acid (4.0 g, 22 mmol, Intermediate 320), HATU (12.5 g, 33 mmol), DIPEA (12 mL, 66 mmol) and DCM (100 mL) were added to a 250 mL one-necked round-bottomed flask. N,O-Dimethylhydroxylamine hydrochloride (2.6 g, 26 mmol) was added under an N2 atmosphere, and the resulting mixture was stirred for 4 h at rt. The reaction was concentrated to dryness under reduced pressure to give the crude product, which was diluted with water (100 mL) and extracted with EtOAc (50 mL×3). The combined organic extracts were washed with brine (50 mL), dried over anhydrous Na₂SO₄, filtered, and concentrated to dryness under reduced pressure to give the crude product that was purified by silica gel chromatography (20-50% EtOAc/petroleum ether) to give the title compound as a colorless oil.

### Intermediate 322

### 3-(2,2,2-Trifluoroethyl)cyclobutane-1-carbaldehyde

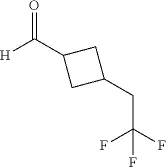 [SMILES: O=CC1CC(CC(F)(F)F)C1]

A 1 M solution of DIBAL-H in toluene (60 mL, 60 mmol) was added dropwise to a stirred solution of N-methoxy-N-methyl-3-(2,2,2-trifluoroethyl)-cyclobutanecarboxamide (4.5 g, 20 mmol, Intermediate 321) in Et₂O (200 mL) at −78° C. The mixture reaction was stirred for 1 h, then warmed to rt and quenched by adding saturated aqueous Rochelle salt (100 mL). After extracting with Et₂O (100 mL×3), the organic layers were combined, washed with brine (100 mL×2), dried over anhydrous Na₂SO₄, filtered, and concentrated to dryness under reduced pressure to give the title compound as a yellow oil, which was used without further purification.

### Intermediate 323

### (R,E)-2-Methyl-N-((3-(2,2,2-trifluoroethyl)cyclobutyl)methylene)propane-2-sulfinamide

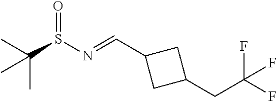 [SMILES: CC(C)(C)[S@@](=O)/N=C/C1CC(CC(F)(F)F)C1]

The title compound was prepared as described for the synthesis of Intermediate 234, using 3-(2,2,2-trifluoroethyl)cyclobutane-1-carbaldehyde (Intermediate 322) in place of 4,4-difluorocyclohexane-1-carbaldehyde. The product was purified by silica gel chromatography (0-20% EtOAc/petroleum ether) to afford the title compound as a colorless oil.

### Intermediate 324

### (E)-1-(2-Methoxyvinyl)-1-(trifluoromethyl)cyclopropane

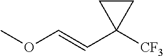

A mixture consisting of (methoxymethyl)triphenylphosphonium chloride (11 g, 33 mmol) in Et₂O (40 mL) was cooled to 0° C. A 1M solution of t-BuOK in t-BuOH (32 mL, 32 mmol) was added dropwise under a N₂ atmosphere. After addition, the orange mixture was stirred for 2 h at 0° C. and 1 h at rt. The solution was then re-cooled to 0° C. and a solution of 1-(trifluoromethyl)cyclopropane-1-carbaldehyde (3.5 g, 25 mmol, as a solution in 51 mL of toluene and 50 mL of Et₂O) was added slowly. After addition, the mixture was stirred for 16 h at rt. After this time, 50 mL of 2 N aqueous HCl was added and the mixture was extracted with Et₂O (2×50 mL). The organic layers were combined, washed with water (60 mL) and brine (60 mL), dried over anhydrous Na₂SO₄, filtered, and concentrated under reduced pressure at 0° C. to give the crude title compound, which was used in the next step directly.

### Intermediate 325

### 2-(1-(Trifluoromethyl)cyclopropyl)acetaldehyde

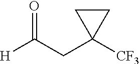

To a solution of (E)-1-(2-methoxyvinyl)-1-(trifluoromethyl)cyclopropane (4.2 g, 25 mmol, Intermediate 324 as a solution in 70 mL toluene and Et₂O) in THF (30 mL), 3 N aqueous HCl (25 mL, 76 mmol) was added. The mixture was stirred for 2 h at 80° C. under N₂. The mixture was then cooled to rt and extracted with Et₂O (50 mL). The organic layer was washed with water (60 mL) and brine (60 mL), dried over anhydrous Na₂SO₄, filtered, and concentrated to about a 45 mL volume under reduced pressure at 0° C. to give the crude title compound which was used in the next step directly.

### Intermediate 326

### (R,E)-2-Methyl-N-(2-(1-(trifluoromethyl)cyclopropyl)ethylidene)propane-2-sulfinamide

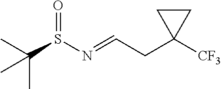 [SMILES: CC(C)(C)[S@@](=O)/N=C/CC1(C(F)(F)F)CC1]

To a solution of 2-(1-(trifluoromethyl)cyclopropyl)acetaldehyde (3.8 g, 25 mmol, Intermediate 325) in 1:1 toluene:THF solution (100 mL) was added 2-methylpropane-2-sulfinamide (3.6 g, 30 mmol), CuSO₄ (16 g, 100 mmol) and PPTS (0.63 g, 2.5 mmol). The resultant solution was allowed to stir at rt for 16 h. After this time, the mixture was filtered and washed with EtOAc (20 mL). The filtrate was separated, washed with water (100 mL) and brine (100 mL), dried over anhydrous Na₂SO₄, filtered, and concentrated to dryness to afford the crude product which was purified by silica gel chromatography (0-20% EtOAc/petroleum ether) to afford the title compound as a colorless solid.

### Intermediate 327

### (R,E)-N-(4,4-Difluoro-3,3-dimethylbutylidene)-2-methylpropane-2-sulfinamide

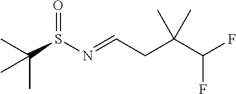

The title compound was prepared as described for the synthesis of Intermediate 234, using 4,4-difluoro-3,3-dimethylbutanal in place of 4,4-difluorocyclohexane-1-carbaldehyde. The filtrate, prior to concentration, was further washed with water (50 mL) and brine (50 mL), dried over anhydrous Na₂SO₄, filtered, and concentrated to dryness to afford the crude product which was purified by silica gel chromatography (0-20% EtOAc/petroleum ether) to afford the title compound as a yellow oil.

### Intermediate 328

### (1R,3s,5S)-6,6-Difluorobicyclo[3.1.0]hexane-3-carbaldehyde and (1R,3r,5S)-6,6-difluorobicyclo[3.1.0]hexane-3-carbaldehyde

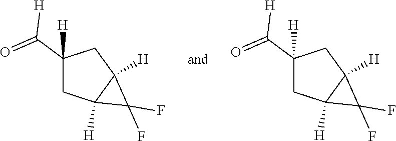 [SMILES: O=C[C@@H]1C[C@@H]2[C@H](C1)C2(F)F ; O=C[C@H]1C[C@@H]2[C@H](C1)C2(F)F]

To a solution of methyl (1R,5S)-6,6-difluorobicyclo[3.1.0]hexane-3-carboxylate (3.4 g, 19 mmol) in DCM (60 mL) at −78° C. was added DIBAL-H (29 mL, 29 mmol, 1 M solution in toluene) dropwise and allowed to stir at rt for 2 h. After this time the reaction was quenched with a saturated aqueous solution of Rochelle Salt (100 mL). The biphasic mixture was transferred to a separatory funnel and extracted with CH₂Cl₂ (50 mL×4). The combined organic layers were washed with brine (50 mL), dried over anhydrous Na₂SO₄, filtered, and concentrated to remove CH₂Cl₂ to afford the crude title compounds as a solution in toluene which was used without further purification.

### Intermediate 329

### (R)—N-((E)-((1R,3s,5S)-6,6-Difluorobicyclo[3.1.0]hexan-3-yl)methylene)-2-methylpropane-2-sulfinamide

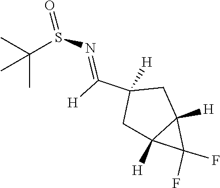 [SMILES: CC(C)(C)[S@@](=O)/N=C/[C@@H]1C[C@@H]2[C@H](C1)C2(F)F]

### Intermediate 330

### (R)—N-((E)-((1R,3r,5S)-6,6-Difluorobicyclo[3.1.0]hexan-3-yl)methylene)-2-methylpropane-2-sulfinamide

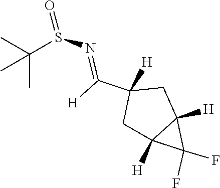 [SMILES: CC(C)(C)[S@@](=O)/N=C/[C@H]1C[C@@H]2[C@H](C1)C2(F)F]

The title compounds were prepared as described for the synthesis of Intermediate 234, using (1R,3s,5S)-6,6-difluorobicyclo[3.1.0]hexane-3-carbaldehyde and (1R,3r,5S)-6,6-difluorobicyclo[3.1.0]hexane-3-carbaldehyde (Intermediate 328) in place of 4,4-difluorocyclohexane-1-carbaldehyde. The products were purified by silica gel chromatography (5-17% EtOAc/petroleum ether) to afford (R)—N-((E)-((1R,3r,5S)-6,6-difluorobicyclo[3.1.0]hexan-3-yl)methylene)-2-methylpropane-2-sulfinamide (Intermediate 330) as the first eluting fraction and (R)—N-((E)-((1R,3s,5S)-6,6-difluorobicyclo[3.1.0]hexan-3-yl)methylene)-2-methylpropane-2-sulfinamide (Intermediate 329) as the second eluting fraction.

### Intermediate 331

### (R)—N-((E)-(5,5-Difluorotetrahydro-2H-pyran-2-yl)methylene)-2-methylpropane-2-sulfinamide

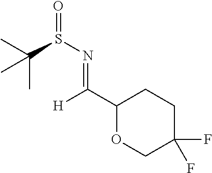 [SMILES: CC(C)(C)[S@@](=O)/N=C/C1CCC(F)(F)CO1]

The title compound was prepared as described for the synthesis of Intermediate 234, using 5,5-difluorotetrahydro-2H-pyran-2-carbaldehyde in place of 4,4-difluorocyclohexane-1-carbaldehyde. The product was purified by silica gel chromatography twice (0-20% EtOAc/petroleum ether) to afford the title compound as a colorless oil.

### Intermediate 332

### (R)-2-Methyl-N-((E)-((S*)-tetrahydro-2H-pyran-2-yl)methylene)propane-2-sulfinamide

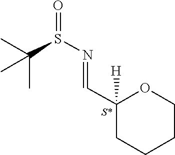

### Intermediate 333

### (R)-2-Methyl-N-((E)-((R*)-tetrahydro-2H-pyran-2-yl)methylene)propane-2-sulfinamide

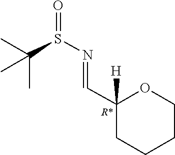

The title compounds were prepared as described for the synthesis of Intermediate 234, using tetrahydro-2H-pyran-2-carbaldehyde in place of 4,4-difluorocyclohexane-1-carbaldehyde. The filtrate was further washed with water (50 mL) and brine (30 mL×2). The combined aqueous layers were then extracted with DCM (30 mL×2), combined with the original filtrate, dried over anhydrous Na₂SO₄, filtered, and concentrated under reduced pressure to afford the crude title compounds. The crude products were purified by silica gel chromatography (0-20% EtOAc/petroleum ether) to afford a mixture of the title compounds which were separated by SFC using a chiral stationary phase (DAICEL CHIRALPAK AD, 10 μm, 250×50 mm, mobile phase. 80% CO₂ in IPA). The first eluting isomer was Intermediate 332 and the second eluting isomer was Intermediate 333. The separated fractions were concentrated under reduced pressure, suspended in water (10 mL), frozen, and lyophilized to dryness to afford Intermediate 332 as a white solid and Intermediate 333 as a colorless oil.

### Intermediate 334

### (1R,3r,5S′)-Bicyclo[3.1.0]hexane-3-carbaldehyde and (1R,3s,5S′)-bicyclo[3.1.0]hexane-3-carbaldehyde

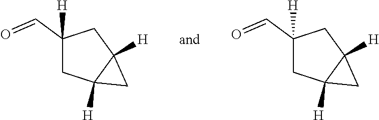 [SMILES: O=C[C@@H]1C[C@@H]2C[C@@H]2C1 ; O=C[C@H]1C[C@@H]2C[C@@H]2C1]

A solution of t-BuOK in THF (40 mL, 40 mmol, 1 M) was added dropwise to a solution of (methoxymethyl)triphenylphosphonium chloride (14 g, 42 mmol) in THF (140 mL) at 0° C. and the resulting solution was stirred at that temperature for 1 h. Then, a solution of bicyclo[3.1.0]hexan-3-one (3.1 g, 32 mmol) in THF (10 mL) was added dropwise and the resultant solution was allowed to warm to rt and stir for 16 h. After this time 2 N aqueous HCl (20 mL) was added followed by petroleum ether (300 mL). The biphasic mixture was washed with water (100 mL) and separated. The organic layer was concentrated to approximately 50 mL and was diluted with THF (140 mL) and 2 N aqueous HCl (20 mL, 40 mmol). The solution was heated to 80° C. and allowed to stir for 2 h. After this time, the reaction was cooled to rt, diluted with petroleum ether (100 mL), and transferred to a separatory funnel. The solution was then washed with water (50 mL) and brine (30 mL). The organic layer was concentrated to about 30 mL under reduced pressure to afford a crude solution of the title compounds in petroleum ether and THF and they were directly used in the next step.

### Intermediate 335

### (R)—N-((E)-((1R,3r,5S)-Bicyclo[3.1.0]hexan-3-yl)methylene)-2-methylpropane-2-sulfinamide

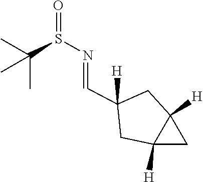

### Intermediate 336

### (R)—N-((E)-((1R,3s,5S)-Bicyclo[3.1.0]hexan-3-yl)methylene)-2-methylpropane-2-sulfinamide

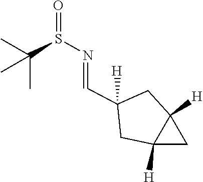

The title compound was prepared as described for the synthesis of Intermediate 234, using (1R,3r,5S)-bicyclo[3.1.0]hexane-3-carbaldehyde and (1R,3r,5S)-bicyclo[3.1.0]hexane-3-carbaldehyde (Intermediate 334) in place of 4,4-difluorocyclohexane-1-carbaldehyde. The crude products were purified by silica gel chromatography (17% EtOAc/petroleum ether) followed by diastereomer separation by SFC using a chiral stationary phase (CHIRAL ART Amylose-C NED, 5 μm, 250×30 mm, mobile phase: 90% CO₂ in 2:1 MeOH:DCM). The first eluting isomer was Intermediate 336 and the second eluting isomer was Intermediate 335. The separated fractions were concentrated under reduced pressure, suspended in water (2 mL), frozen, and lyophilized to dryness to afford Intermediate 335 as a off-white solid and Intermediate 336 as a yellow oil.

### Intermediate 337

### 2-(3,3-Difluorocyclobutoxy)acetic acid

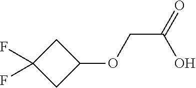 [SMILES: O=C(O)COC1CC(F)(F)C1]

NaH (7.4 g, 185 mmol, 60% in mineral oil) was added to a 500 mL three-necked round-bottomed flask equipped with a mechanical stirrer, condensing tube, and thermometer, charged with a solution of 3,3-difluorocyclobutanol (10 g, 93 mmol) in THF (300 mL) at 0° C. The reaction mixture was stirred at that temperature for 1 h, followed by the addition of 2-bromoacetic acid (12.8 g, 92.5 mmol) portion-wise as a solution in THF (50 mL). The mixture was stirred for 1 h at 0° C., followed by 15 min at rt, and then heated to 70° C. for 12 h. After this time, the reaction was cooled to rt and water (300 mL) was added slowly. The biphasic solution was transferred to a separatory funnel and extracted with CH₂Cl₂ (100 mL×2). The organic layer was discarded. The aqueous layer was acidified with 2 N aqueous HCl to pH ˜3-4 and extracted with EtOAc (100 mL×3). The combined organic extracts were washed with brine (300 mL), dried over anhydrous Na₂SO₄, filtered, and concentrated to dryness under reduced pressure to give a crude product, which was purified by silica gel chromatography (25-50% EtOAc/petroleum ether) to afford the title compound as a yellow oil.

### Intermediate 338

### 2-(3,3-Difluorocyclobutoxy)-N-methoxy-N-methylacetamide

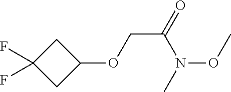

2-(3,3-Difluorocyclobutoxy)acetic acid (12 g, 72 mmol, Intermediate 337), HATU (41 g, 108 mmol), DIPEA (39 mL, 217 mmol) and N,O-dimethylhydroxylamine hydrochloride (8.5 g, 87 mmol) were dissolved in DCM (200 mL) in a 500 mL three-necked round-bottomed flask equipped with a mechanical stirrer, condensing tube, and thermometer, and the resulting mixture was stirred for 16 h at rt. After this time, water (300 mL) was added and the mixture extracted with CH₂Cl₂ (100 mL×3). The combined organic extracts were washed with brine (200 mL), dried over anhydrous Na₂SO₄, filtered, and concentrated to dryness under reduced pressure to give a crude product, which was purified by silica gel chromatography (20-50% EtOAc/petroleum ether) to give the title compound as a colorless oil.

### Intermediate 339

### 2-(3,3-Difluorocyclobutoxy)acetaldehyde

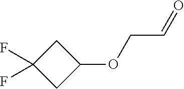

The title compound was prepared as described for the synthesis of Intermediate 322, using 2-(3,3-difluorocyclobutoxy)-N-methoxy-N-methylacetamide (Intermediate 338) in place of N-methoxy-N-methyl-3-(2,2,2-trifluoroethyl)cyclobutane-1-carboxamide. The crude title compound was used directly in the next step without purification.

### Intermediate 340

### (R,E)-N-(2-(3,3-Difluorocyclobutoxy)ethylidene)-2-methylpropane-2-sulfinamide

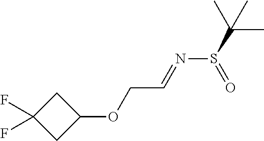 [SMILES: CC(C)(C)[S@@](=O)/N=C/COC1CC(F)(F)C1]

The title compound was prepared as described for the synthesis of Intermediate 234, using 2-(3,3-difluorocyclobutoxy)acetaldehyde (Intermediate 339) in place of 4,4-difluorocyclohexane-1-carbaldehyde. The crude product was purified by silica gel chromatography (0-20% EtOAc/petroleum ether) to afford the title compound as a colorless oil.

### Intermediate 341

### (1R,5S,6r)-3,3-Difluoro-N-methoxy-N-methylbicyclo[3.1.0]hexane-6-carboxamide

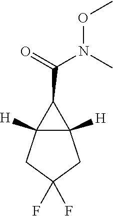

The title compound was prepared as described for the synthesis of Intermediate 338, using (1R,5S,6r)-3,3-difluorobicyclo[3.1.0]hexane-6-carboxylic acid in place of 2-(3,3-difluorocyclobutoxy)acetic acid. The crude product was purified by silica gel chromatography (0-50% EtOAc/petroleum ether) to afford the title compound as a pale-yellow oil.

### Intermediate 342

### (1R,5S,6r)-3,3-Difluorobicyclo[3.1.0]hexane-6-carbaldehyde

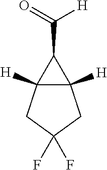

To a stirred solution of (1R,5S,6r)-3,3-difluoro-N-methoxy-N-methylbicyclo[3.1.0]hexane-6-carboxamide (7.0 g, 34 mmol, Intermediate 341) in THF (70 mL) was added a solution of DIBAL-H (57 mL, 86 mmol, 1.5 M in toluene) dropwise at −78° C., maintaining an internal temperature of not more than −70° C., under N₂ and the resulting mixture was allowed to stir at −78° C. for 1 h. After this time, the mixture was warmed to 0° C. and saturated aqueous Rochelle salt (100 mL) was added and stirred for 1 h. The mixture was then separated and the organic layer was washed with brine (100 mL), dried over anhydrous Na₂SO₄, and filtered. The solution of the crude title compound in ˜130 mL THE and toluene was used in the next step without further manipulation.

### Intermediate 343

### (R)—N-((E)-((1R,5S,6r)-3,3-Difluorobicyclo[3.1.0]hexan-6-yl)methylene)-2-methylpropane-2-sulfinamide

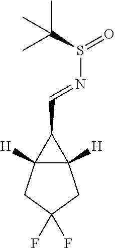 [SMILES: CC(C)(C)[S@@](=O)/N=C/[C@@H]1[C@H]2CC(F)(F)C[C@@H]12]

The title compound was prepared as described for the synthesis of Intermediate 234, using (1R,5S,6r)-3,3-difluorobicyclo[3.1.0]hexane-6-carbaldehyde (Intermediate 342) in place of 4,4-difluorocyclohexane-1-carbaldehyde. The crude product was purified by silica gel chromatography (0-40% EtOAc/petroleum ether) to afford the title compound as a pale-yellow oil.

### Intermediate 344

### (R)—N—((S)-1-(6-((R)-Amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide

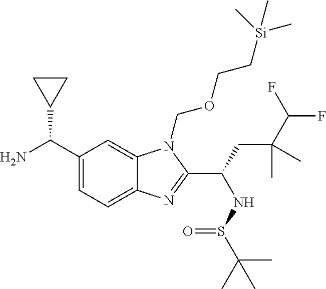 [SMILES: CC(C)(C[C@H](N[S@](=O)C(C)(C)C)c1nc2ccc([C@H](N)C3CC3)cc2n1COCC[Si](C)(C)C)C(F)F]

To a solution of (R)-cyclopropyl(1-((2-(trimethylsilyl)ethoxy)methyl)-1H-benzo[d]imidazol-6-yl)methanamine (100 mg, 0.32 mmol, Intermediate 186) in THF (4 mL) at −78° C. under N₂ was added a solution of n-BuLi (0.2 mL, 0.5 mmol, 2.5 M in hexanes). The mixture was stirred at that temperature for 0.5 h and then (R,E)-N-(4,4-difluoro-3,3-dimethylbutylidene)-2-methylpropane-2-sulfinamide (105 mg, 0.44 mmol, Intermediate 327) in THF (2 mL) was added to the reaction mixture. The reaction was stirred at −78° C. for 1 h. After this time, the reaction was quenched with saturated aqueous NH₄Cl (60 mL), transferred to a separatory funnel, and extracted with EtOAc (60 mL×2). The combined organic layers were washed with brine (60 mL), dried over anhydrous Na₂SO₄, filtered, and concentrated to dryness under reduced pressure to give the crude title compound as a yellow oil which was used without further purification.

### Intermediate 345

### (R)—N—((S)-1-(6-((R)-Amino(cyclopropyl)methyl)-1-((2-(trimethylsilyl)ethoxy)methyl)-1H-benzo[d]imidazol-2-yl)-2-((R)-3,3-difluorocyclopentyl)ethyl)-2-methylpropane-2-sulfinamide

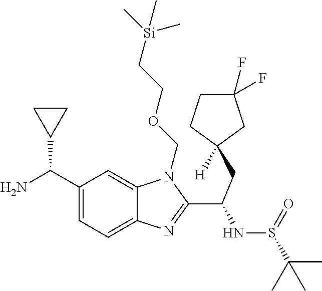 [SMILES: CC(C)(C)[S@@](=O)N[C@@H](C[C@H]1CCC(F)(F)C1)c1nc2ccc([C@H](N)C3CC3)cc2n1COCC[Si](C)(C)C]

The title compound was prepared as described for the synthesis of Intermediate 344, using (R)—N-((E)-2-((R)-3,3-difluorocyclopentyl)ethylidene)-2-methylpropane-2-sulfinamide (Intermediate 393 in place of (R,E)-N-(4,4-difluoro-3,3-dimethylbutylidene)-2-methylpropane-2-sulfinamide.

### Intermediate 346

### (R)—N—((S)-(6-((R)-Amino(cyclopropyl)methyl)-1-((2-(trimethylsilyl)ethoxy)methyl)-1H-benzo[d]imidazol-2-yl)((1R,3s,5S)-6,6-difluorobicyclo[3.1.0]hexan-3-yl)methyl)-2-methylpropane-2-sulfinamide

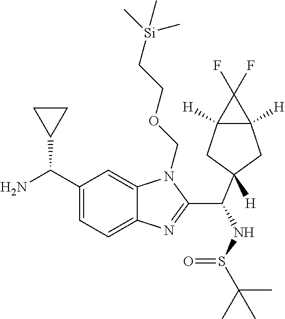 [SMILES: CC(C)(C)[S@@](=O)N[C@H](c1nc2ccc([C@H](N)C3CC3)cc2n1COCC[Si](C)(C)C)[C@@H]1C[C@@H]2[C@H](C1)C2(F)F]

The title compound was prepared as described for the synthesis of Intermediate 344, using (R)—N-((E)-((1R,3s,5S)-6,6-difluorobicyclo[3.1.0]hexan-3-yl)methylene)-2-methylpropane-2-sulfinamide (Intermediate 329) in place of (R,E)-N-(4,4-difluoro-3,3-dimethylbutylidene)-2-methylpropane-2-sulfinamide.

### Intermediate 347

### (R)—N—((S)-(6-((R)-Amino(cyclopropyl)methyl)-1-((2-(trimethylsilyl)ethoxy)methyl)-1H-benzo[d]imidazol-2-yl)((1R,3r,5S)-6,6-difluorobicyclo[3.1.0]hexan-3-yl)methyl)-2-methylpropane-2-sulfinamide

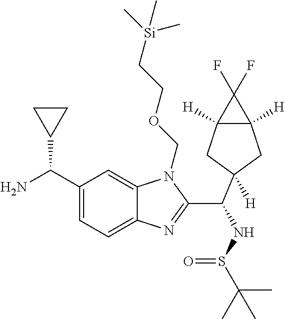 [SMILES: CC(C)(C)[S@@](=O)N[C@H](c1nc2ccc([C@H](N)C3CC3)cc2n1COCC[Si](C)(C)C)[C@H]1C[C@@H]2[C@H](C1)C2(F)F]

The title compound was prepared as described for the synthesis of Intermediate 344, using (R)—N-((E)-((1R,3r,5S)-6,6-difluorobicyclo[3.1.0]hexan-3-yl)methylene)-2-methylpropane-2-sulfinamide (Intermediate 330) in place of (R,E)-N-(4,4-difluoro-3,3-dimethylbutylidene)-2-methylpropane-2-sulfinamide.

### Intermediate 348

### (R)—N—((S)-1-(6-((R)-Amino(cyclopropyl)methyl)-1-((2-(trimethylsilyl)ethoxy)methyl)-1H-benzo[d]imidazol-2-yl)-2-(1-(trifluoromethyl)cyclopropyl)ethyl)-2-methylpropane-2-sulfinamide

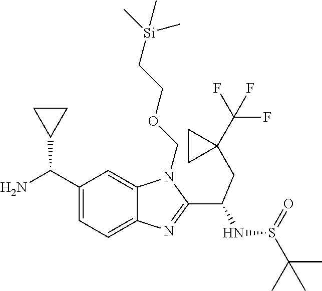 [SMILES: CC(C)(C)[S@@](=O)N[C@@H](CC1(C(F)(F)F)CC1)c1nc2ccc([C@H](N)C3CC3)cc2n1COCC[Si](C)(C)C]

The title compound was prepared as described for the synthesis of Intermediate 344, using (R,E)-2-methyl-N-(2-(1-(trifluoromethyl)cyclopropyl)ethylidene)propane-2-sulfinamide (Intermediate 326) in place of (R,E)-N-(4,4-difluoro-3,3-dimethylbutylidene)-2-methylpropane-2-sulfinamide.

### Intermediate 349

### (R)—N—((S)-(6-((R)-Amino(cyclopropyl)methyl)-1-((2-(trimethylsilyl)ethoxy)methyl)-1H-benzo[d]imidazol-2-yl)(3-(2,2,2-trifluoroethyl)cyclobutyl)methyl)-2-methylpropane-2-sulfinamide

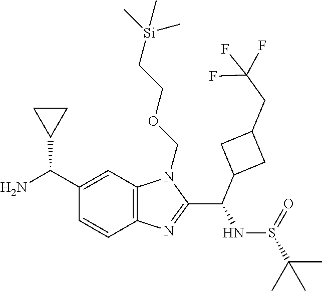 [SMILES: CC(C)(C)[S@@](=O)N[C@H](c1nc2ccc([C@H](N)C3CC3)cc2n1COCC[Si](C)(C)C)C1CC(CC(F)(F)F)C1]

The title compound was prepared as described for the synthesis of Intermediate 344, using (R,E)-2-methyl-N-((3-(2,2,2-trifluoroethyl)cyclobutyl)methylene)propane-2-sulfinamide (Intermediate 323) in place of (R,E)-N-(4,4-difluoro-3,3-dimethylbutylidene)-2-methylpropane-2-sulfinamide.

### Intermediate 350

### (R)—N—((S)-(6-((R)-Amino(cyclopropyl)methyl)-1-((2-(trimethylsilyl)ethoxy)methyl)-1H-benzo[d]imidazol-2-yl)((R)-3,3-difluorocyclohexyl)methyl)-2-methylpropane-2-sulfinamide

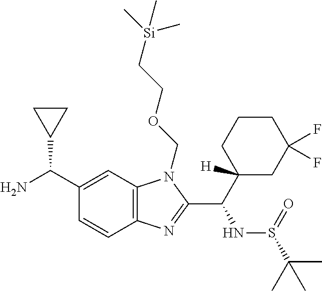 [SMILES: CC(C)(C)[S@@](=O)N[C@H](c1nc2ccc([C@H](N)C3CC3)cc2n1COCC[Si](C)(C)C)[C@@H]1CCCC(F)(F)C1]

The title compound was prepared as described for the synthesis of Intermediate 344, using (R)—N-((E)-((R)-3,3-difluorocyclohexyl)methylene)-2-methylpropane-2-sulfinamide (Intermediate 136) in place of (R,E)-N-(4,4-difluoro-3,3-dimethylbutylidene)-2-methylpropane-2-sulfinamide.

### Intermediate 351

### (R)—N—((S)-(6-((R)-Amino(cyclopropyl)methyl)-1-((2-(trimethylsilyl)ethoxy)methyl)-1H-benzo[d]imidazol-2-yl)((S)-3,3-difluorocyclohexyl)methyl)-2-methylpropane-2-sulfinamide

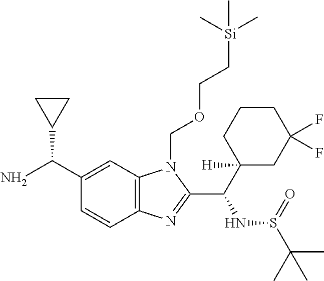 [SMILES: CC(C)(C)[S@@](=O)N[C@H](c1nc2ccc([C@H](N)C3CC3)cc2n1COCC[Si](C)(C)C)[C@H]1CCCC(F)(F)C1]

The title compound was prepared as described for the synthesis of Intermediate 344, using (R)—N-((E)-((S)-3,3-difluorocyclohexyl)methylene)-2-methylpropane-2-sulfinamide (Intermediate 140) in place of (R,E)-N-(4,4-difluoro-3,3-dimethylbutylidene)-2-methylpropane-2-sulfinamide.

### Intermediate 352

### (R)—N-((6-((R)-Amino(cyclopropyl)methyl)-1-((2-(trimethylsilyl)ethoxy)methyl)-1H-benzo[d]imidazol-2-yl)((S*)-tetrahydro-2H-pyran-2-yl)methyl)-2-methylpropane-2-sulfinamide

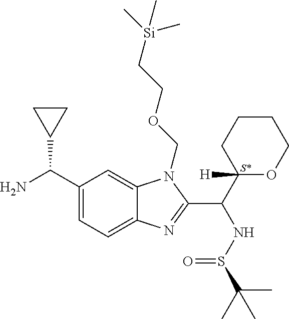 [SMILES: CC(C)(C)[S@@](=O)NC(c1nc2ccc([C@H](N)C3CC3)cc2n1COCC[Si](C)(C)C)[C@@H]1CCCCO1]

The title compound was prepared as described for the synthesis of Intermediate 344, using (R)-2-methyl-N-((E)-((S*)-tetrahydro-2H-pyran-2-yl)methylene)propane-2-sulfinamide (Intermediate 332) in place of (R,E)-N-(4,4-difluoro-3,3-dimethylbutylidene)-2-methylpropane-2-sulfinamide.

### Intermediate 353

### (R)—N-((6-((R)-Amino(cyclopropyl)methyl)-1-((2-(trimethylsilyl)ethoxy)methyl)-1H-benzo[d]imidazol-2-yl)((R*)-tetrahydro-2H-pyran-2-yl)methyl)-2-methylpropane-2-sulfinamide

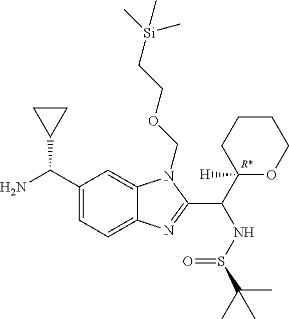 [SMILES: CC(C)(C)[S@@](=O)NC(c1nc2ccc([C@H](N)C3CC3)cc2n1COCC[Si](C)(C)C)[C@H]1CCCCO1]

The title compound was prepared as described for the synthesis of Intermediate 344, using (R)-2-methyl-N-((E)-((R*)-tetrahydro-2H-pyran-2-yl)methylene)propane-2-sulfinamide (Intermediate 333) in place of (R,E)-N-(4,4-difluoro-3,3-dimethylbutylidene)-2-methylpropane-2-sulfinamide.

### Intermediate 354

### (R)—N—((S)-(6-((R)-Amino(cyclopropyl)methyl)-1-((2-(trimethylsilyl)ethoxy)methyl)-1H-benzo[d]imidazol-2-yl)((1R,3s,5S)-bicyclo[3.1.0]hexan-3-yl)methyl)-2-methylpropane-2-sulfinamide

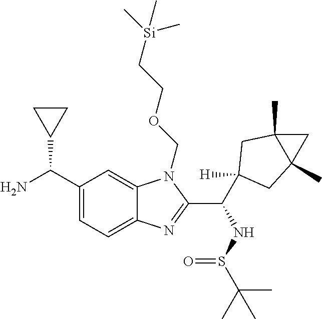 [SMILES: CC(C)(C)[S@@](=O)N[C@H](c1nc2ccc([C@H](N)C3CC3)cc2n1COCC[Si](C)(C)C)[C@@H]1C[C@@]2(C)C[C@@]2(C)C1]

The title compound was prepared as described for the synthesis of Intermediate 344, using (R)—N-((E)-((1R,3s,5S)-bicyclo[3.1.0]hexan-3-yl)methylene)-2-methylpropane-2-sulfinamide (Intermediate 336) in place of (R,E)-N-(4,4-difluoro-3,3-dimethylbutylidene)-2-methylpropane-2-sulfinamide.

### Intermediate 355

### (R)—N—((R)-1-(6-((R)-Amino(cyclopropyl)methyl)-1-((2-(trimethylsilyl)ethoxy)methyl)-1H-benzo[d]imidazol-2-yl)-2-(3,3-difluorocyclobutoxy)ethyl)-2-methylpropane-2-sulfinamide

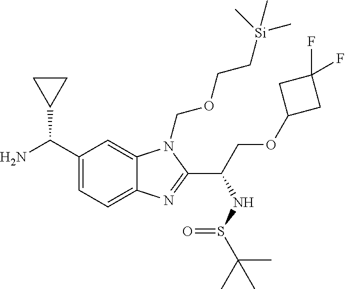 [SMILES: CC(C)(C)[S@@](=O)N[C@@H](COC1CC(F)(F)C1)c1nc2ccc([C@H](N)C3CC3)cc2n1COCC[Si](C)(C)C]

The title compound was prepared as described for the synthesis of Intermediate 344, using (R,E)-N-(2-(3,3-difluorocyclobutoxy)ethylidene)-2-methylpropane-2-sulfinamide (Intermediate 340) in place of (R,E)-N-(4,4-difluoro-3,3-dimethylbutylidene)-2-methylpropane-2-sulfinamide.

### Intermediate 356

### (R)—N—(Cyclopropyl(1-((2-(trimethylsilyl)ethoxy)methyl)-1H-benzo[d]imidazol-5-yl)methyl)-2-(3,3-difluorocyclobutyl)acetamide

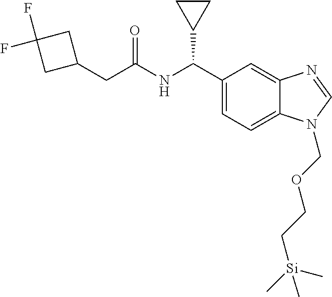 [SMILES: C[Si](C)(C)CCOCn1cnc2cc([C@H](NC(=O)CC3CC(F)(F)C3)C3CC3)ccc21]

To a solution of (R)-cyclopropyl(1-((2-(trimethylsilyl)ethoxy)methyl)-1H-benzo[d]imidazol-5-yl)methanamine (1.0 g, 3.2 mmol, Intermediate 248) in MeCN (10 mL), 2-(3,3-difluorocyclobutyl)acetic acid (615 mg, 4.09 mmol), EDCI (1.2 g, 6.3 mmol), HOBt (851 mg, 6.30 mmol) and DIPEA (1.6 g, 13 mmol) were added sequentially. The mixture was stirred at rt overnight. After this time, the reaction was quenched with a saturated aqueous solution of NH₄Cl (10 mL) and extracted with CH₂Cl₂ (20 mL×3). The combined organic layers were washed with a saturated aqueous solution of NaHCO₃ (10 mL), dried over anhydrous Na₂SO₄, and concentrated under reduced pressure to give the crude product. Silica gel chromatography (0-5% MeOH/DCM) afforded the title compound as a white solid.

### Intermediate 357

### N—((R)-(2-((R)—(((R)-tert-Butylsulfinyl)amino)((S*)-5,5-difluorotetrahydro-2H-pyran-2-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide

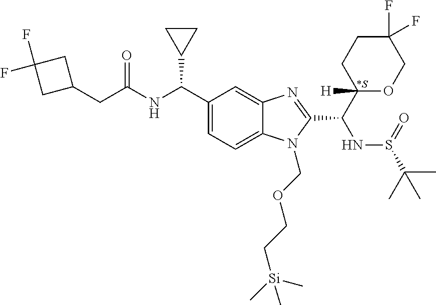 [SMILES: CC(C)(C)[S@@](=O)N[C@H](c1nc2cc([C@H](NC(=O)CC3CC(F)(F)C3)C3CC3)ccc2n1COCC[Si](C)(C)C)[C@@H]1CCC(F)(F)CO1]

### Intermediate 358

### N—((R)-(2-((R)—(((R)-tert-Butylsulfinyl)amino)((R*)-5,5-difluorotetrahydro-2H-pyran-2-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide

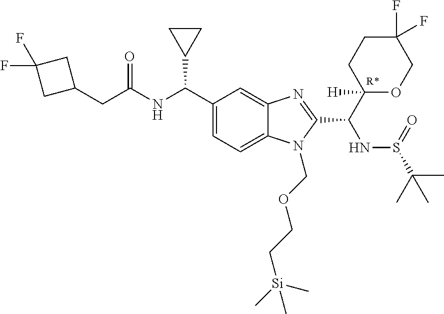 [SMILES: CC(C)(C)[S@@](=O)N[C@H](c1nc2cc([C@H](NC(=O)CC3CC(F)(F)C3)C3CC3)ccc2n1COCC[Si](C)(C)C)[C@H]1CCC(F)(F)CO1]

The title compounds were prepared as described for the synthesis of Intermediate 344, using (R)—N-((E)-(5,5-difluorotetrahydro-2H-pyran-2-yl)methylene)-2-methylpropane-2-sulfinamide (Intermediate 331) in place of (R,E)-N-(4,4-difluoro-3,3-dimethylbutylidene)-2-methylpropane-2-sulfinamide. The two diastereomers were separated by silica gel chromatography (0-2% MeOH/DCM) affording the title compounds. Intermediate 358 was the first eluting isomer as a yellow oil and Intermediate 357 was the second eluting isomer as a yellow oil.

### Intermediate 359

### (R)—N—((S)-(5-((R)-Amino(cyclopropyl)methyl)-1-((2-(trimethylsilyl)ethoxy)methyl)-1H-benzo[d]imidazol-2-yl)((R)-3,3-difluorocyclohexyl)methyl)-2-methylpropane-2-sulfinamide

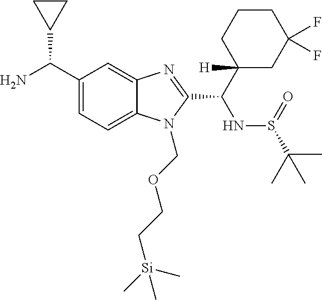 [SMILES: CC(C)(C)[S@@](=O)N[C@H](c1nc2cc([C@H](N)C3CC3)ccc2n1COCC[Si](C)(C)C)[C@@H]1CCCC(F)(F)C1]

The title compound was prepared as described for the synthesis of Intermediate 344, using (R)—N-((E)-((R)-3,3-difluorocyclohexyl)methylene)-2-methylpropane-2-sulfinamide (Intermediate 136) in place of (R,E)-N-(4,4-difluoro-3,3-dimethylbutylidene)-2-methylpropane-2-sulfinamide and (R)-cyclopropyl(1-((2-(trimethylsilyl)ethoxy)methyl)-1H-benzo[d]imidazol-5-yl)methanamine (Intermediate 248) in place of (R)-cyclopropyl(1-((2-(trimethylsilyl)ethoxy)methyl)-1H-benzo[d]imidazol-6-yl)methanamine.

### Intermediate 360

### N—((R)-(2-((S)-1-(((R)-tert-Butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide

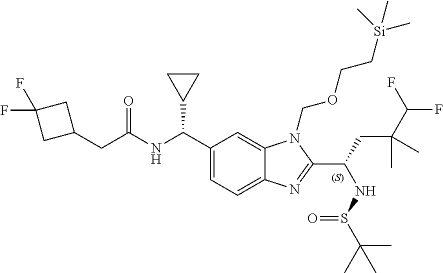

EDCI (62 mg, 0.33 mmol) was added to a solution of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide (190 mg, 0.23 mmol, 68% purity, Intermediate 344), 2-(3,3-difluorocyclobutyl)acetic acid (70 mg, 0.46 mmol), HOBt (41 mg, 0.30 mmol) and DIPEA (0.16 mL, 0.93 mmol) in MeCN (10 mL) and the resulting mixture was allowed to stir at rt overnight. After this time, water (20 mL) was added followed by CH₂Cl₂ (20 mL). The mixture was separated and the aqueous layer was extracted with CH₂Cl₂ (3×15 mL). The combined organic layers were washed with water (20 mL) and brine (20 mL), dried over anhydrous Na₂SO₄, filtered, and concentrated under reduced pressure to afford the crude product. Purification by silica gel chromatography (0-100% EtOAc/petroleum ether) afforded the title compound as a yellow oil.

### Intermediate 361

### N—((R)-(2-((S)-1-Amino-4,4-difluoro-3,3-dimethylbutyl)-1H-benzo[d]imidazol-6-yl)(cyclopropyl)methyl)-2-(3,3-difluorocyclobutyl)acetamide

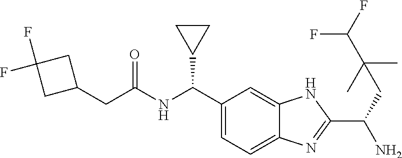 [SMILES: CC(C)(C[C@H](N)c1nc2ccc([C@H](NC(=O)CC3CC(F)(F)C3)C3CC3)cc2[nH]1)C(F)F]

A 4 M solution of HCl in 1,4-dioxane (5 mL) was added to a solution of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide (140 mg, 0.14 mmol, 71% purity, Intermediate 360) in 1,4-dioxane (5 mL). The reaction was heated to 55° C. and stirred for 2 h. After this time, the solution was concentrated to about half its total volume. Water was added and the aqueous layer was washed with petroleum ether (2×10 mL). The aqueous layer was brought to a pH ˜10 by the addition of 3 M aqueous NaOH. The aqueous layer was then extracted with CH₂Cl₂ (3×15 mL), and the combined organic layers were dried over anhydrous Na₂SO₄, filtered, and concentrated under reduced pressure to provide the title compound as a yellow oil that was used in the next step without further purification.

### Intermediate 362

### N—((R)-(2-((S)-1-(((R)-tert-Butylsulfinyl)amino)-2-((R)-3,3-difluorocyclopentyl)ethyl)-1-((2-(trimethylsilyl)ethoxy)methyl)-1H-benzo[d]imidazol-6-yl)(cyclopropyl)methyl)-2-(3,3-difluorocyclobutyl)acetamide

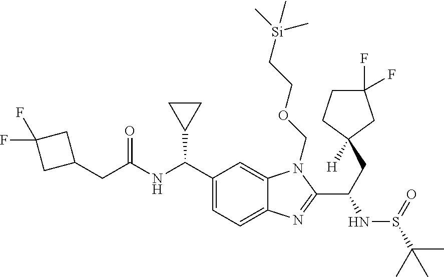 [SMILES: CC(C)(C)[S@@](=O)N[C@@H](C[C@H]1CCC(F)(F)C1)c1nc2ccc([C@H](NC(=O)CC3CC(F)(F)C3)C3CC3)cc2n1COCC[Si](C)(C)C]

The title compound was prepared as described for the synthesis of Intermediate 360, using (R)—N—((S)-1-(6-((R)-amino(cyclopropyl)methyl)-1-((2-(trimethylsilyl)ethoxy)methyl)-1H-benzo[d]imidazol-2-yl)-2-((R)-3,3-difluorocyclopentyl)ethyl)-2-methylpropane-2-sulfinamide (Intermediate 345) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 363

### N—((R)-(2-((S)-1-Amino-2-((R)-3,3-difluorocyclopentyl)ethyl)-1H-benzo[d]imidazol-6-yl)(cyclopropyl)methyl)-2-(3,3-difluorocyclobutyl)acetamide

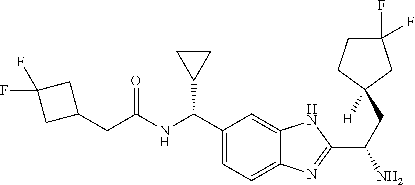 [SMILES: N[C@@H](C[C@H]1CCC(F)(F)C1)c1nc2ccc([C@H](NC(=O)CC3CC(F)(F)C3)C3CC3)cc2[nH]1]

The title compound was prepared as described for the synthesis of Intermediate 361, using N—((R)-(2-((S)-1-(((R)-tert-butylsulfinyl)amino)-2-((R)-3,3-difluorocyclopentyl)ethyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 362) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide.

### Intermediate 364

### N—((R)-(2-((S)—(((R)-tert-Butylsulfinyl)amino)((1R,3s,5S)-6,6-difluorobicyclo[3.1.0]hexan-3-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide

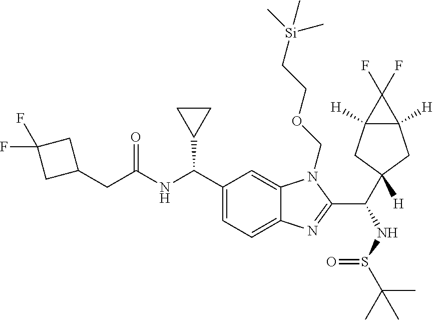 [SMILES: CC(C)(C)[S@@](=O)N[C@H](c1nc2ccc([C@H](NC(=O)CC3CC(F)(F)C3)C3CC3)cc2n1COCC[Si](C)(C)C)[C@@H]1C[C@@H]2[C@H](C1)C2(F)F]

The title compound was prepared as described for the synthesis of Intermediate 360, using (R)—N—((S)-(6-((R)-amino(cyclopropyl)methyl)-1-((2-(trimethylsilyl)ethoxy)methyl)-1H-benzo[d]imidazol-2-yl)((1R,3s,5S)-6,6-difluorobicyclo[3.1.0]hexan-3-yl)methyl)-2-methylpropane-2-sulfinamide (Intermediate 346) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 365

### N—((R)-(2-((S)-Amino((1R,3s,5S)-6,6-difluorobicyclo[3.1.0]hexan-3-yl)methyl)-1H-benzo[d]imidazol-6-yl)(cyclopropyl)methyl)-2-(3,3-difluorocyclobutyl)acetamide

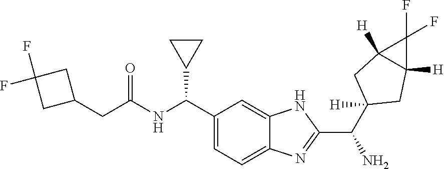 [SMILES: N[C@H](c1nc2ccc([C@H](NC(=O)CC3CC(F)(F)C3)C3CC3)cc2[nH]1)[C@@H]1C[C@@H]2[C@H](C1)C2(F)F]

The title compound was prepared as described for the synthesis of Intermediate 361, using N—((R)-(2-((S)—(((R)-tert-butylsulfinyl)amino)((1R,3s,5S)-6,6-difluorobicyclo[3.1.0]hexan-3-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 364) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide.

### Intermediate 366

### N—((R)-(2-((S)—(((R)-tert-Butylsulfinyl)amino)((1R,3r,5S)-6,6-difluorobicyclo[3.1.0]hexan-3-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide

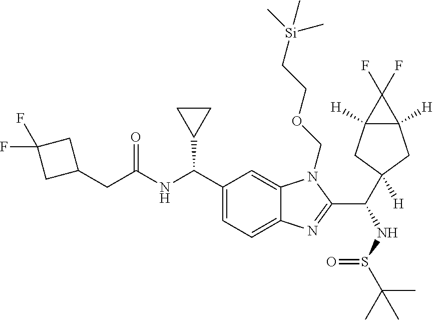 [SMILES: CC(C)(C)[S@@](=O)N[C@H](c1nc2ccc([C@H](NC(=O)CC3CC(F)(F)C3)C3CC3)cc2n1COCC[Si](C)(C)C)[C@H]1C[C@@H]2[C@H](C1)C2(F)F]

The title compound was prepared as described for the synthesis of Intermediate 360, using (R)—N—((S)-(6-((R)-amino(cyclopropyl)methyl)-1-((2-(trimethylsilyl)ethoxy)methyl)-1H-benzo[d]imidazol-2-yl)((1R,3r,5S)-6,6-difluorobicyclo[3.1.0]hexan-3-yl)methyl)-2-methylpropane-2-sulfinamide (Intermediate 347) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 367

### N—((R)-(2-((S)-Amino((1R,3r,5S)-6,6-difluorobicyclo[3.1.0]hexan-3-yl)methyl)-1H-benzo[d]imidazol-6-yl)(cyclopropyl)methyl)-2-(3,3-difluorocyclobutyl)acetamide-hydrogen chloride

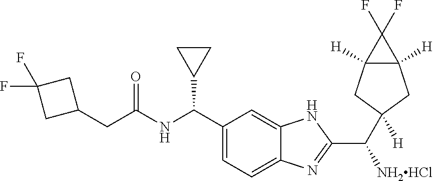

The title compound was prepared as described for the synthesis of Intermediate 361, using N—((R)-(2-((S)—(((R)-tert-butylsulfinyl)amino)((1R,3r,5S)-6,6-difluorobicyclo[3.1.0]hexan-3-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 366) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide. After stirring at 55° C. for 2 h, the reaction mixture was concentrated to dryness and purified by preparatory HPLC (Venusil ASB Phenyl, 5 μm, 150×30 mm column; mobile phase: 24-54% MeCN in aqueous HCl (0.005 N)) to afford the title compound as a white solid.

### Intermediate 368

### N—((R)-(2-((S)-1-(((R)-tert-Butylsulfinyl)amino)-2-(1-(trifluoromethyl)cyclopropyl)ethyl)-1-((2-(trimethylsilyl)ethoxy)methyl)-1H-benzo[d]imidazol-6-yl)(cyclopropyl)methyl)-2-(3,3-difluorocyclobutyl)acetamide

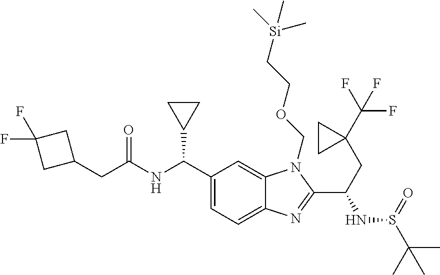 [SMILES: CC(C)(C)[S@@](=O)N[C@@H](CC1(C(F)(F)F)CC1)c1nc2ccc([C@H](NC(=O)CC3CC(F)(F)C3)C3CC3)cc2n1COCC[Si](C)(C)C]

The title compound was prepared as described for the synthesis of Intermediate 360, using (R)—N—((S)-1-(6-((R)-amino(cyclopropyl)methyl)-1-((2-(trimethylsilyl)ethoxy)methyl)-1H-benzo[d]imidazol-2-yl)-2-(1-(trifluoromethyl)cyclopropyl)ethyl)-2-methylpropane-2-sulfinamide (Intermediate 348) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 369

### N—((R)-(2-((S)-1-Amino-2-(1-(trifluoromethyl)cyclopropyl)ethyl)-1H-benzo[d]imidazol-6-yl)(cyclopropyl)methyl)-2-(3,3-difluorocyclobutyl)acetamide

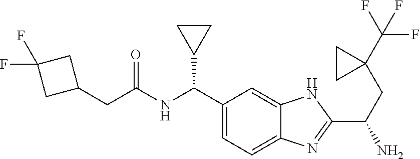 [SMILES: N[C@@H](CC1(C(F)(F)F)CC1)c1nc2ccc([C@H](NC(=O)CC3CC(F)(F)C3)C3CC3)cc2[nH]1]

The title compound was prepared as described for the synthesis of Intermediate 361, using N—((R)-(2-((S)-1-(((R)-tert-butylsulfinyl)amino)-2-(1-(trifluoromethyl)cyclopropyl)ethyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 368) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide.

### Intermediate 370

### N—((R)-(2-((S)—(((R)-tert-Butylsulfinyl)amino)(3-(2,2,2-trifluoroethyl)cyclobutyl)methyl)-1-((2-(trimethylsilyl)ethoxy)methyl)-1H-benzo[d]imidazol-6-yl)(cyclopropyl)methyl)-2-(3,3-difluorocyclobutyl)acetamide

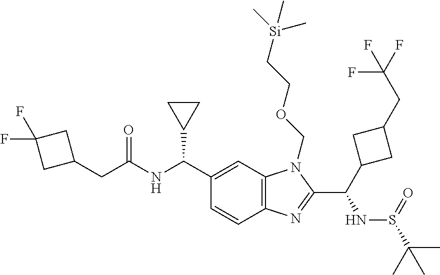 [SMILES: CC(C)(C)[S@@](=O)N[C@H](c1nc2ccc([C@H](NC(=O)CC3CC(F)(F)C3)C3CC3)cc2n1COCC[Si](C)(C)C)C1CC(CC(F)(F)F)C1]

The title compound was prepared as described for the synthesis of Intermediate 360, using (R)—N—((S)-(6-((R)-amino(cyclopropyl)methyl)-1-((2-(trimethylsilyl)ethoxy)methyl)-1H-benzo[d]imidazol-2-yl)(3-(2,2,2-trifluoroethyl)cyclobutyl)methyl)-2-methylpropane-2-sulfinamide (Intermediate 349) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 371

### N—((R)-(2-((S)-Amino(3-(2,2,2-trifluoroethyl)cyclobutyl)methyl)-1H-benzo[d]imidazol-6-yl)(cyclopropyl)methyl)-2-(3,3-difluorocyclobutyl)acetamide hydrochloride

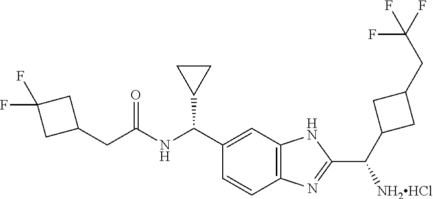

The title compound was prepared as described for the synthesis of Intermediate 361, using N—((R)-(2-((S)—(((R)-tert-butylsulfinyl)amino)(3-(2,2,2-trifluoroethyl)cyclobutyl)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 370) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide. The reaction was concentrated to dryness to afford the crude HCl salt in place of undergoing a basic aqueous work-up.

### Intermediate 372

### N—((R)-(2-((S)—(((R)-tert-Butylsulfinyl)amino)((R)-3,3-difluorocyclohexyl)methyl)-1-((2-(trimethylsilyl)ethoxy)methyl)-1H-benzo[d]imidazol-6-yl)(cyclopropyl)methyl)-2-(3,3-difluorocyclobutyl)acetamide

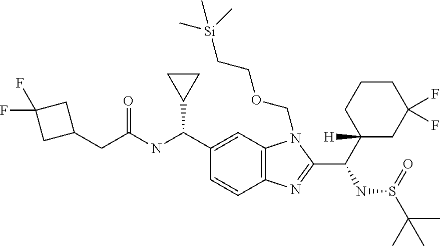 [SMILES: CC(C)(C)[S@@](=O)N[C@H](c1nc2ccc([C@H](NC(=O)CC3CC(F)(F)C3)C3CC3)cc2n1COCC[Si](C)(C)C)[C@@H]1CCCC(F)(F)C1]

The title compound was prepared as described for the synthesis of Intermediate 360, using (R)—N—((S)-(6-((R)-amino(cyclopropyl)methyl)-1-((2-(trimethylsilyl)ethoxy)methyl)-1H-benzo[d]imidazol-2-yl)((R)-3,3-difluorocyclohexyl)methyl)-2-methylpropane-2-sulfinamide (Intermediate 350) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 373

### N—((R)-(2-((S)-Amino((R)-3,3-difluorocyclohexyl)methyl)-1H-benzo[d]imidazol-6-yl)(cyclopropyl)methyl)-2-(3,3-difluorocyclobutyl)acetamide

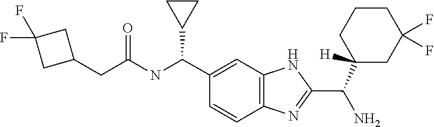

The title compound was prepared as described for the synthesis of Intermediate 361, using N—((R)-(2-((S)—(((R)-tert-butylsulfinyl)amino)((R)-3,3-difluorocyclohexyl)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 372) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide.

### Intermediate 374

### N—((R)-(2-((S)—(((R)-tert-Butylsulfinyl)amino)((S)-3,3-difluorocyclohexyl)methyl)-1-((2-(trimethylsilyl)ethoxy)methyl)-1H-benzo[d]imidazol-6-yl)(cyclopropyl)methyl)-2-(3,3-difluorocyclobutyl)acetamide

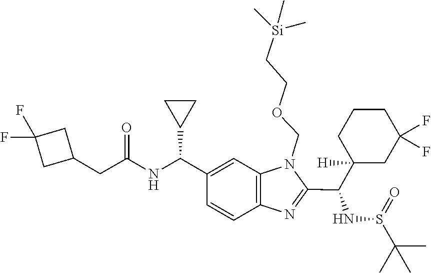 [SMILES: CC(C)(C)[S@@](=O)N[C@H](c1nc2ccc([C@H](NC(=O)CC3CC(F)(F)C3)C3CC3)cc2n1COCC[Si](C)(C)C)[C@H]1CCCC(F)(F)C1]

The title compound was prepared as described for the synthesis of Intermediate 360, using (R)—N—((S)-(6-((R)-amino(cyclopropyl)methyl)-1-((2-(trimethylsilyl)ethoxy)methyl)-1H-benzo[d]imidazol-2-yl)((S)-3,3-difluorocyclohexyl)methyl)-2-methylpropane-2-sulfinamide (Intermediate 351) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 375

### N—((R)-(2-((S)-Amino((S)-3,3-difluorocyclohexyl)methyl)-1H-benzo[d]imidazol-6-yl)(cyclopropyl)methyl)-2-(3,3-difluorocyclobutyl)acetamide

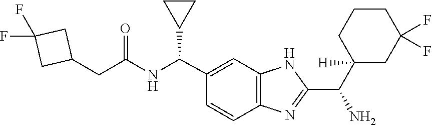 [SMILES: N[C@H](c1nc2ccc([C@H](NC(=O)CC3CC(F)(F)C3)C3CC3)cc2[nH]1)[C@H]1CCCC(F)(F)C1]

The title compound was prepared as described for the synthesis of Intermediate 361, using N—((R)-(2-((S)—(((R)-tert-butylsulfinyl)amino)((S)-3,3-difluorocyclohexyl)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 374) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide.

### Intermediate 376

### N—((R)-(2-((R)—(((R)-tert-Butylsulfinyl)amino)((S*)-tetrahydro-2H-pyran-2-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide

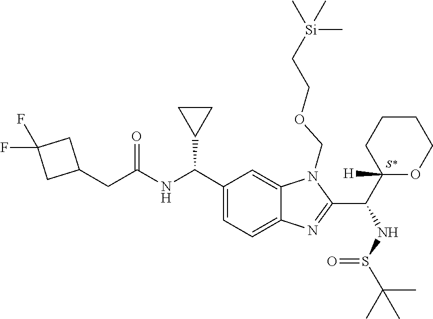 [SMILES: CC(C)(C)[S@@](=O)N[C@H](c1nc2ccc([C@H](NC(=O)CC3CC(F)(F)C3)C3CC3)cc2n1COCC[Si](C)(C)C)[C@@H]1CCCCO1]

The title compound was prepared as described for the synthesis of Intermediate 360, using (R)—N-((6-((R)-amino(cyclopropyl)methyl)-1-((2-(trimethylsilyl)ethoxy)methyl)-1H-benzo[d]imidazol-2-yl)((S*)-tetrahydro-2H-pyran-2-yl)methyl)-2-methylpropane-2-sulfinamide (Intermediate 352) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 377

### N—((R)-(2-((R)-Amino((S*)-tetrahydro-2H-pyran-2-yl)methyl)-1H-benzo[d]imidazol-6-yl)(cyclopropyl)methyl)-2-(3,3-difluorocyclobutyl)acetamide hydrochloride

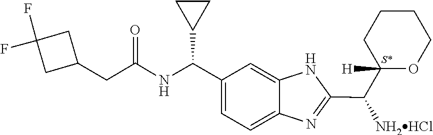

The title compound was prepared as described for the synthesis of intermediate 361, using N—((R)-(2-((R)-(((R)-tert-butylsulfinyl)amino)((S*)-tetrahydro-2H-pyran-2-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 376) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide, and in place of undergoing an aqueous work-up and freebased, the reaction was concentrated to dryness to afford the HCl salt.

### Intermediate 378

### N—((R)-(2-((R)-(((R)-tert-Butylsulfinyl)amino)((R*)-tetrahydro-2H-pyran-2-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide

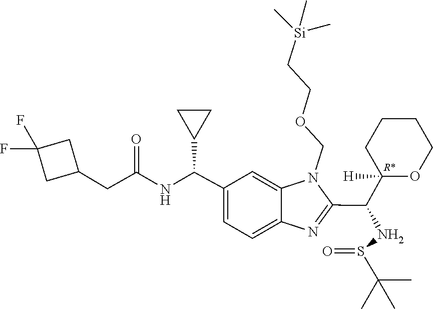

The title compound was prepared as described for the synthesis of Intermediate 360, using (R)—N-((6-((R)-amino(cyclopropyl)methyl)-1-((2-(trimethylsilyl)ethoxy)methyl)-1H-benzo[d]imidazol-2-yl)((R*)-tetrahydro-2H-pyran-2-yl)methyl)-2-methylpropane-2-sulfinamide (Intermediate 353) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 379

### N—((R)-(2-((R)-Amino((R*)-tetrahydro-2H-pyran-2-yl)methyl)-1H-benzo[d]imidazol-6-yl)(cyclopropyl)methyl)-2-(3,3-difluorocyclobutyl)acetamide hydrochloride

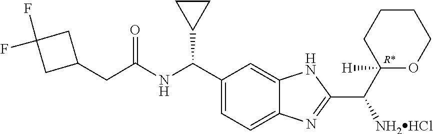

The title compound was prepared as described for the synthesis of Intermediate 361, using N—((R)-(2-((R)—(((R)-tert-butylsulfinyl)amino)((R*)-tetrahydro-2H-pyran-2-yl)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 378) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide.

### Intermediate 380

### N—((R)-(2-((S)—(((R)-tert-Butylsulfinyl)amino)((R)-3,3-difluorocyclohexyl)methyl)-1-((2-(trimethylsilyl)ethoxy)methyl)-1H-benzo[d]imidazol-5-yl)(cyclopropyl)methyl)-4,4,4-trifluoro-3-methylbutanamide

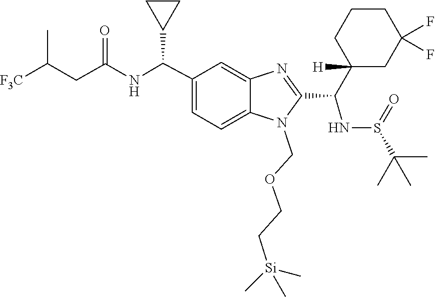 [SMILES: CC(CC(=O)N[C@@H](c1ccc2c(c1)nc([C@@H](N[S@](=O)C(C)(C)C)[C@@H]1CCCC(F)(F)C1)n2COCC[Si](C)(C)C)C1CC1)C(F)(F)F]

The title compound was prepared as described for the synthesis of Intermediate 360, using (R)—N—((S)-(5-((R)-amino(cyclopropyl)methyl)-1-((2-(trimethylsilyl)ethoxy)methyl)-1H-benzo[d]imidazol-2-yl)((R)-3,3-difluorocyclohexyl)methyl)-2-methylpropane-2-sulfinamide (Intermediate 359) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide and 4,4,4-trifluoro-3-methylbutanoic acid in place of 2-(3,3-difluorocyclobutyl)acetic acid.

### Intermediate 381

### N—((R)-(2-((S)-Amino((R)-3,3-difluorocyclohexyl)methyl)-1H-benzo[d]imidazol-5-yl)(cyclopropyl)methyl)-4,4,4-trifluoro-3-methylbutanamide hydrochloride

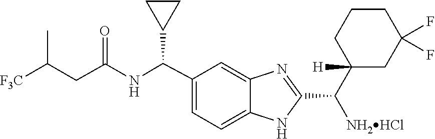

The title compound was prepared as described for the synthesis of Intermediate 361, using N—((R)-(2-((S)—(((R)-tert-butylsulfinyl)amino)((R)-3,3-difluorocyclohexyl)methyl)-1-((2-(trimethylsilyl)ethoxy)methyl)-1H-benzo[d]imidazol-5-yl)(cyclopropyl)methyl)-4,4,4-trifluoro-3-methylbutanamide (Intermediate 380) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide, and in place of undergoing an aqueous work-up and freebased, the reaction was concentrated to dryness to afford the HCl salt.

### Intermediate 382

### N—((R)-(2-((S)-((1R,3s,5S)-Bicyclo[3.1.0]hexan-3-yl)(((R)-tert-butylsulfinyl)amino)methyl)-1-((2-(trimethylsilyl)ethoxy)methyl)-1H-benzo[d]imidazol-6-yl)(cyclopropyl)methyl)-2-(3,3-difluorocyclobutyl)acetamide

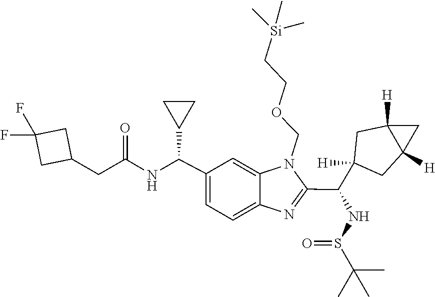 [SMILES: CC(C)(C)[S@@](=O)N[C@H](c1nc2ccc([C@H](NC(=O)CC3CC(F)(F)C3)C3CC3)cc2n1COCC[Si](C)(C)C)[C@@H]1C[C@H]2C[C@H]2C1]

The title compound was prepared as described for the synthesis of Intermediate 360, using (R)—N—((S)-(6-((R)-amino(cyclopropyl)methyl)-1-((2-(trimethylsilyl)ethoxy)methyl)-1H-benzo[d]imidazol-2-yl)((1R,3s,5S)-bicyclo[3.1.0]hexan-3-yl)methyl)-2-methylpropane-2-sulfinamide (Intermediate 354) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 383

### N—((R)-(2-((S)-Amino((1R,3s,5S)-bicyclo[3.1.0]hexan-3-yl)methyl)-1H-benzo[d]imidazol-6-yl)(cyclopropyl)methyl)-2-(3,3-difluorocyclobutyl)acetamide

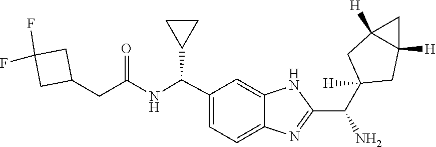 [SMILES: N[C@H](c1nc2ccc([C@H](NC(=O)CC3CC(F)(F)C3)C3CC3)cc2[nH]1)[C@@H]1C[C@H]2C[C@H]2C1]

The title compound was prepared as described for the synthesis of Intermediate 361, using N—((R)-(2-((S)-((1R,3s,5S)-bicyclo[3.1.0]hexan-3-yl)(((R)-tert-butylsulfinyl)amino)methyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 382) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide.

### Intermediate 384

### N—((R)-(2-((R)-1-(((R)-tert-Butylsulfinyl)amino)-2-(3,3-difluorocyclobutoxy)ethyl)-1-((2-(trimethylsilyl)ethoxy)methyl)-1H-benzo[d]imidazol-6-yl)(cyclopropyl)methyl)-2-(3,3-difluorocyclobutyl)acetamide

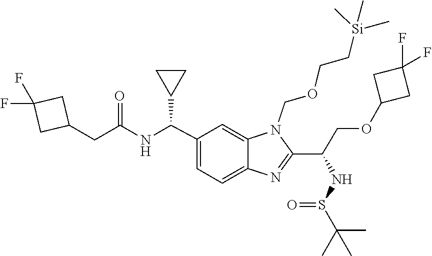 [SMILES: CC(C)(C)[S@@](=O)N[C@@H](COC1CC(F)(F)C1)c1nc2ccc([C@H](NC(=O)CC3CC(F)(F)C3)C3CC3)cc2n1COCC[Si](C)(C)C]

The title compound was prepared as described for the synthesis of Intermediate 360, using (R)—N—((R)-1-(6-((R)-amino(cyclopropyl)methyl)-1-((2-(trimethylsilyl)ethoxy)methyl)-1H-benzo[d]imidazol-2-yl)-2-(3,3-difluorocyclobutoxy)ethyl)-2-methylpropane-2-sulfinamide (Intermediate 355) in place of (R)—N—((S)-1-(6-((R)-amino(cyclopropyl)methyl)-1-((2-(trimethylsilyl)ethoxy)methyl)-1H-benzo[d]imidazol-2-yl)-4,4-difluoro-3,3-dimethylbutyl)-2-methylpropane-2-sulfinamide.

### Intermediate 385

### N—((R)-(2-((R)-1-Amino-2-(3,3-difluorocyclobutoxy)ethyl)-1H-benzo[d]imidazol-6-yl)(cyclopropyl)methyl)-2-(3,3-difluorocyclobutyl)acetamide

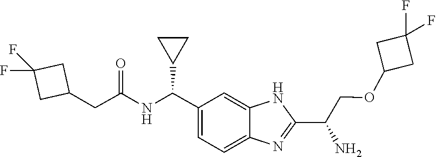 [SMILES: N[C@@H](COC1CC(F)(F)C1)c1nc2ccc([C@H](NC(=O)CC3CC(F)(F)C3)C3CC3)cc2[nH]1]

The title compound was prepared as described for the synthesis of Intermediate 361, using N—((R)-(2-((R)-1-(((R)-tert-butylsulfinyl)amino)-2-(3,3-difluorocyclobutoxy)ethyl)-1-((2-(trimethylsilyl)ethoxy)methyl)-1H-benzo[d]imidazol-6-yl)(cyclopropyl)methyl)-2-(3,3-difluorocyclobutyl)acetamide (Intermediate 384) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide.

### Intermediate 386

### N—((R)-(2-((R)-Amino((S*)-5,5-difluorotetrahydro-2H-pyran-2-yl)methyl)-1H-benzo[d]imidazol-6-yl)(cyclopropyl)methyl)-2-(3,3-difluorocyclobutyl)acetamide hydrochloride

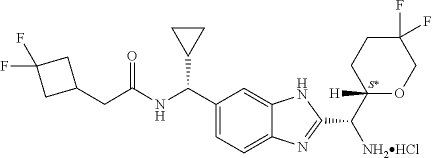

The title compound was prepared as described for the synthesis of Intermediate 361, using N—((R)-(2-((R)—(((R)-tert-butylsulfinyl)amino)((S*)-5,5-difluorotetrahydro-2H-pyran-2-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide (Intermediate 357) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide, and in place of undergoing an aqueous work-up and freebased, the reaction was concentrated to dryness to afford the HCl salt.

### Intermediate 387

### N—((R)-(2-((R)-Amino((R*)-5,5-difluorotetrahydro-2H-pyran-2-yl)methyl)-1H-benzo[d]imidazol-6-yl)(cyclopropyl)methyl)-2-(3,3-difluorocyclobutyl)acetamide hydrochloride

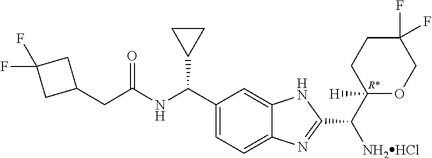

The title compound was prepared as described for the synthesis of Intermediate 361, using N—((R)-(2-((R)—(((R)-tert-butylsulfinyl)amino)((R*)-5,5-difluorotetrahydro-2H-pyran-2-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide (Intermediate 358) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide, and in place of undergoing a basic aqueous work-up, the reaction was concentrated to dryness to afford the HCl salt.

### Intermediate 388

### Dimethyl (S)-2-(3-oxocyclopentyl)malonate

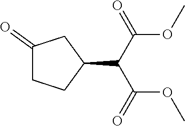

A mixture of (S)-1-(pyrrolidin-2-ylmethyl)pyrrolidine (15.4 g, 100 mmol), TFA (7.4 mL, 100 mmol) and MeOH (100 mL) were stirred at rt for 20 min to provide the catalyst solution (110 mL). Cyclopent-2-en-1-one (70 g, 853 mmol), dimethyl malonate (195 mL, 1.7 mol), a stir bar and MeOH (800 mL) were added to a 2 L round-bottomed flask, followed by the addition of the catalyst solution (85.4 mL), and the resultant mixture was stirred at rt for 48 h. The reaction mixture was concentrated to dryness in vacuo, and the residue was purified by silica gel chromatography (5-9% EtOAc/petroleum ether) to afford the title compound as a yellow oil.

### Intermediate 389

### Dimethyl (S)-2-(3,3-difluorocyclopentyl)malonate

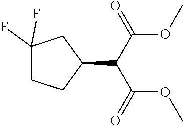 [SMILES: COC(=O)C(C(=O)OC)[C@H]1CCC(F)(F)C1]

Dimethyl (S)-2-(3-oxocyclopentyl)malonate (80 g, 373 mmol, Intermediate 388), a stir bar and 1,2-dichloroethane (700 mL) were added to a 2 L round-bottomed flask, followed by the addition of Deoxo-fluor® (206 mL, 1.12 mol), and the resulting mixture was stirred at 70° C. for 5 h. The reaction mixture was cooled to rt, slowly added to cold (0° C.) saturated aqueous NaHCO₃ (2 L), and then extracted with DCM (3×500 mL). The combined organic extracts were dried over anhydrous MgSO₄, filtered, and concentrated to dryness in vacuo. The residue was purified by silica gel chromatography (6-9% EtOAc/petroleum ether) to afford the title compound as a yellow oil.

### Intermediate 390

### (R)-2-(3,3-Difluorocyclopentyl)acetic acid

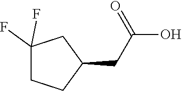 [SMILES: O=C(O)C[C@H]1CCC(F)(F)C1]

NaOH (44 g, 1.1 mol), a stir bar and MeOH (600 mL) were added to a 2 L round-bottomed flask, and the resulting mixture stirred until homogeneous before adding dimethyl (S)-2-(3,3-difluorocyclopentyl)malonate (65 g, 275 mmol, Intermediate 389). The reaction mixture was stirred at rt for 48 h before cooling to 0° C., and treating with HCl (300 mL, 1.2 mol, 4 M in 1,4-dioxane) dropwise via syringe over the course of 30 min. Stirring was continued for 10 min before the mixture was filtered through a pad of Celite® and the filtrate concentrated to dryness in vacuo. The residue was dissolved in MeCN (500 mL) and the precipitate was removed by vacuum filtration. The filtrate was added to a 1 L round-bottomed flask containing Cu₂O (3.93 g, 27.5 mmol) and a stir bar, and the resulting mixture stirred at reflux for 12 h before concentrating to dryness in vacuo. The residue was acidified with 1 N aqueous HCl (300 mL), and the resulting mixture extracted with EtOAc (3×500 mL). The combined organic extracts were dried over anhydrous Na₂SO₄, filtered, and concentrated to dryness in vacuo. The crude product was purified by silica gel chromatography (9-33% EtOAc/petroleum ether) to afford the title compound as a yellow oil.

### Intermediate 391

### (S)-4-Benzyl-3-(2-((R)-3,3-difluorocyclopentyl)acetyl)oxazolidin-2-one

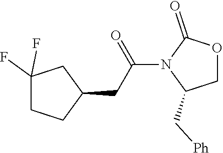 [SMILES: O=C(C[C@H]1CCC(F)(F)C1)N1C(=O)OC[C@@H]1Cc1ccccc1]

(R)-2-(3,3-difluorocyclopentyl)acetic acid (18.5 g, 113 mmol, Intermediate 390), (S)-4-phenyloxazolidin-2-one (22 g, 124 mmol), N,N′-diisopropylmethanediimine (17.1 g, 136 mmol), DMAP (2.1 g, 17 mmol), a stir bar and DCM (300 mL) were added to a 1 L round-bottomed flask, and the resulting mixture stirred at rt for 12 h. The mixture was filtered through a pad of Celite® and the filtrate concentrated to dryness in vacuo. The crude product was purified by silica gel chromatography (5-17% EtOAc/petroleum ether) to afford the title compound as a yellow oil.

### Intermediate 392

### (R)-2-(3,3-Difluorocyclopentyl)ethan-1-ol

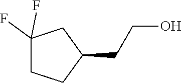

THF (100 mL) and a stir bar were added to a 500 mL three-necked round-bottomed flask fitted with an addition funnel, followed by the addition of LiAlH₄ (3.5 g, 93 mmol), and the reaction vessel was cooled to 0° C. A solution consisting of (S)-4-benzyl-3-(2-((R)-3,3-difluorocyclopentyl)acetyl)oxazolidin-2-one (15 g, 46 mmol, Intermediate 391) and THF (50 mL) was then added dropwise via syringe over the course of 30 min, and stirring was continued at rt for 2 h. The reaction mixture was cooled to 0° C., treated portion-wise with an excess of Na₂SO₄·10H₂O, and stirring continued for 30 min at 0° C. before filtering through a pad of Celite©.

The filtrate was concentrated to dryness under reduced pressure and the crude product purified by silica gel chromatography (5-9% EtOAc/petroleum ether) to afford the title compound as a yellow oil.

### Intermediate 393

### (R)—N-((E)-2-((R)-3,3-difluorocyclopentyl)ethylidene)-2-methylpropane-2-sulfinamide

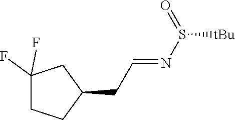 [SMILES: CC(C)(C)[S@@](=O)/N=C/C[C@H]1CCC(F)(F)C1]

(R)-2-(3,3-Difluorocyclopentyl)ethan-1-ol (5.9 g, 39 mmol, Intermediate 392), a stir bar and DCM (60 mL) were added to a 250 mL three-necked round-bottomed flask, and the reaction vessel was cooled to 0° C. Dess-Martin Periodinane (20 g, 47 mmol) was then added in portions over the course of 20 min, and the resulting mixture warmed to 35° C. and stirred for 1 h. The mixture was cooled to rt, diluted with Et₂O (200 mL), and the resultant precipitate removed by vacuum filtration. The filtrate was washed with saturated aqueous Na₂S₂O₃ (200 mL) and saturated aqueous NaHCO₃ (200 mL), dried over anhydrous Na₂SO₄, and re-filtered. The filtrate was transferred to a 500 mL round-bottomed flask containing a stir bar, (R)-2-methylpropane-2-sulfinamide (5.7 g, 47 mmol), CuSO₄ (31.5 g, 197 mmol) and PPTS (1.5 g, 5.9 mmol), and the resulting mixture was stirred at rt for 16 h. The reaction mixture was filtered through a pad of Celite®, and the filtrate concentrated to dryness in vacuo. The crude product was purified by silica gel chromatography (9-33% EtOAc/petroleum ether) to afford the title compound as a yellow oil.

### Intermediate 394

### (R)-Cyclopropyl(1-((2-(trimethylsilyl)ethoxy)methyl)-1H-benzo[d]imidazol-5-yl)methanamine hydrochloride and (R)-cyclopropyl(1-((2-(trimethylsilyl)ethoxy)methyl)-1H-benzo[d]imidazol-6-yl)methanamine hydrochloride

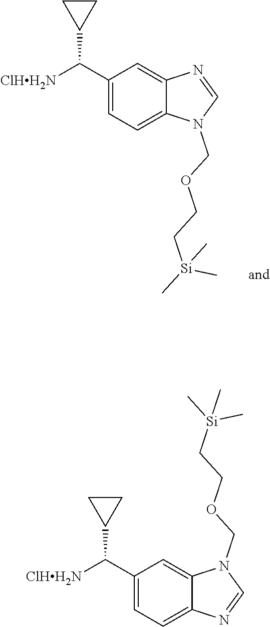

A solution of 1:1 (S)—N—((R)-cyclopropyl(1-((2-(trimethylsilyl)ethoxy)methyl)-1H-benzo[d]imidazol-5-yl)methyl)-2-methylpropane-2-sulfinamide and (S)—N—((R)-cyclopropyl(1-((2-(trimethylsilyl)ethoxy)methyl)-1H-benzo[d]imidazol-6-yl)methyl)-2-methylpropane-2-sulfinamide (535 mg, 1.3 mmol, Intermediates 243 and 244) in DCM (6.3 mL) was treated with 2 N HCl in diethyl ether (0.63 mL, 1.3 mmol) and the resulting mixture was stirred at room temperature for 30 min. The mixture was extracted into water and the aqueous layer was frozen and lyophilized to afford the title compounds as a white solid.

### Intermediate 395

### (R)—N-(Cyclopropyl(1-((2-(trimethylsilyl)ethoxy)methyl)-1H-benzo[d]imidazol-5-yl)methyl)-4,4,4-trifluorobutanamide and (R)—N-(cyclopropyl(1-((2-(trimethylsilyl)ethoxy)methyl)-1H-benzo[d]imidazol-6-yl)methyl)-4,4,4-trifluorobutanamide

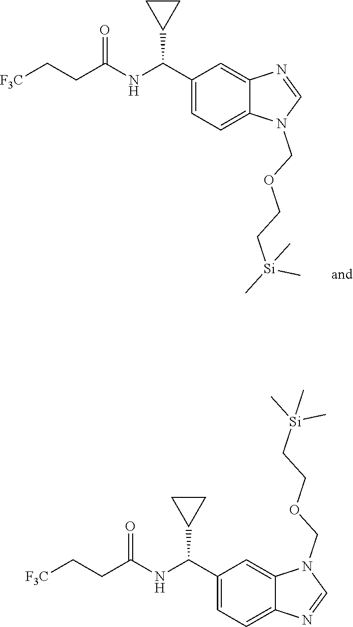 [SMILES: C[Si](C)(C)CCOCn1cnc2cc([C@H](NC(=O)CCC(F)(F)F)C3CC3)ccc21 ; C[Si](C)(C)CCOCn1cnc2ccc([C@H](NC(=O)CCC(F)(F)F)C3CC3)cc21]

A vial was charged with (R)-cyclopropyl(1-((2-(trimethylsilyl)ethoxy)methyl)-1H-benzo[d]imidazol-5-yl)methanamine hydrochloride and (R)-cyclopropyl(1-((2-(trimethylsilyl)ethoxy)methyl)-1H-benzo[d]imidazol-6-yl)methanamine hydrochloride (430 mg, 1.4 mmol, Intermediate 394), DMF (25 mL), HOBt (298 mg, 1.9 mmol), EDCI (342 mg, 1.7 mmol), DIPEA (0.7 mL, 4.1 mmol) and 4,4,4-trifluorobutyric acid (295 mg, 2.1 mmol). The reaction mixture was stirred for 18 h at rt. After that time, the reaction mixture was poured over water and diluted with EtOAc. The layers were separated, and the aqueous phase was further extracted with EtOAc (2×5 mL). The combined organic layers were washed with brine, dried over anhydrous MgSO₄, filtered, and concentrated to dryness. The crude material was purified by silica gel chromatography (0-100% EtOAc (with 10% MeOH)/hexanes). The product containing fractions were concentrated to dryness, redissolved in minimal ACN/water, frozen and lyophilized to afford the mixture of title compounds as a white solid.

### Intermediate 396

### N-((1R)-(2-((1S)-((tert-Butylsulfinyl)amino)((R)-3,3-difluorocyclohexyl)methyl)-1-((2-(trimethylsilyl)ethoxy)methyl)-1H-benzo[d]imidazol-5-yl)(cyclopropyl)methyl)-4,4,4-trifluorobutanamide and N-((1R)-(2-((1S)-((tert-butylsulfinyl)amino)((R)-3,3-difluorocyclohexyl)methyl)-1-((2-(trimethylsilyl)ethoxy)methyl)-1H-benzo[d]imidazol-6-yl)(cyclopropyl)methyl)-4,4,4-trifluorobutanamide

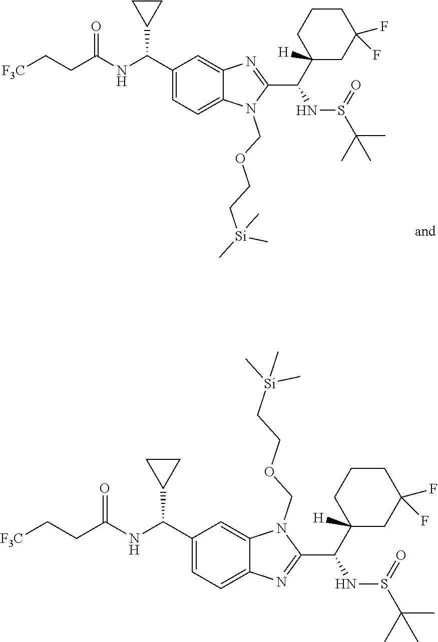 [SMILES: CC(C)(C)S(=O)N[C@H](c1nc2cc([C@H](NC(=O)CCC(F)(F)F)C3CC3)ccc2n1COCC[Si](C)(C)C)[C@@H]1CCCC(F)(F)C1 ; CC(C)(C)S(=O)N[C@H](c1nc2ccc([C@H](NC(=O)CCC(F)(F)F)C3CC3)cc2n1COCC[Si](C)(C)C)[C@@H]1CCCC(F)(F)C1]

An oven dried round bottom flask under nitrogen containing a mixture of (R)—N-(cyclopropyl(1-((2-(trimethylsilyl)ethoxy)methyl)-1H-benzo[d]imidazol-5-yl)methyl)-4,4,4-trifluorobutanamide and (R)—N-(cyclopropyl(1-((2-(trimethylsilyl)ethoxy)methyl)-1H-benzo[d]imidazol-6-yl)methyl)-4,4,4-trifluorobutanamide (185 mg, 0.42 mmol, Intermediate395) in THF (4 mL) was cooled to −78° C. and treated with LDA (3 mL, 1.2 mmol, 0.4 M in THF). After 30 min, the mixture was treated with (R)—N-((E)-((R)-3,3-difluorocyclohexyl)methylene)-2-methylpropane-2-sulfinamide (117 mg, 0.47 mmol, Intermediate 136) as a solution in THF (2 mL). The reaction was then slowly warmed to rt, and the mixture was quenched with water and extracted into 3/1 EtOAc/hexanes. The combined organics were washed with brine, dried over anhydrous MgSO₄, filtered, and concentrated to dryness. The crude material was purified by silica gel chromatography (0-100% EtOAc (with 10% MeOH)/hexanes). The product containing fractions were concentrated to dryness, redissolved in minimal ACN/water, frozen and lyophilized to afford the mixture of title compounds as a white solid.

### Intermediate 397

### N—((R)-(2-((S)-Amino((R)-3,3-difluorocyclohexyl)methyl)-1-((2-(trimethylsilyl)ethoxy)methyl)-1H-benzo[d]imidazol-5-yl)(cyclopropyl)methyl)-4,4,4-trifluorobutanamide and N—((R)-(2-((S)-amino((R)-3,3-difluorocyclohexyl)methyl)-1-((2-(trimethylsilyl)ethoxy)methyl)-1H-benzo[d]imidazol-6-yl)(cyclopropyl)methyl)-4,4,4-trifluorobutanamide

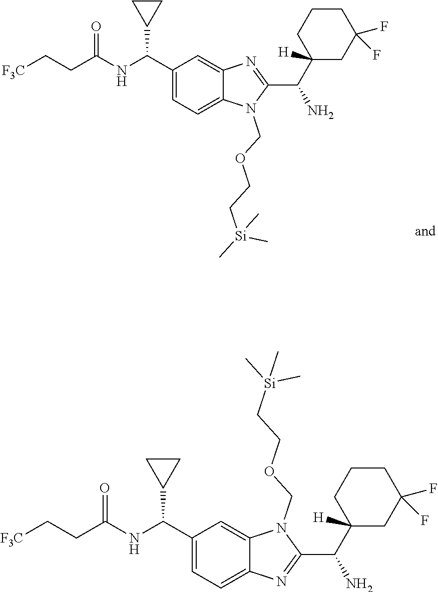 [SMILES: C[Si](C)(C)CCOCn1c([C@@H](N)[C@@H]2CCCC(F)(F)C2)nc2cc([C@H](NC(=O)CCC(F)(F)F)C3CC3)ccc21 ; C[Si](C)(C)CCOCn1c([C@@H](N)[C@@H]2CCCC(F)(F)C2)nc2ccc([C@H](NC(=O)CCC(F)(F)F)C3CC3)cc21]

To a solution of N-((1R)-(2-((1S)-((tert-butylsulfinyl)amino)((R)-3,3-difluorocyclohexyl)methyl)-1-((2-(trimethylsilyl)ethoxy)methyl)-1H-benzo[d]imidazol-5-yl)(cyclopropyl)methyl)-4,4,4-trifluorobutanamide and N-((1R)-(2-((1S)-((tert-butylsulfinyl)amino)((R)-3,3-difluorocyclohexyl)methyl)-1-((2-(trimethylsilyl)ethoxy)methyl)-1H-benzo[d]imidazol-6-yl)(cyclopropyl)methyl)-4,4,4-trifluorobutanamide (76 mg, 0.11 mmol, Intermediate 396) in DCM (1.3 mL) in a small vial was added 2 N HCl in diethyl ether (0.7 mL, 1.3 mmol). After stirring at rt for 1 h, the reaction was quenched with water and the aqueous layer was washed twice with ethyl acetate (wash discarded). The aqueous layer was frozen and lyophilized followed by neutralization with aqueous NaHCO₃ and extraction into EtOAc. The organic layer was dried over anhydrous MgSO₄, filtered, and concentrated to afford the mixture of title compounds as a solid.

### Intermediate 398

### N—((S)-(5-((R)-Cyclopropyl(4,4,4-trifluorobutanamido)methyl)-1-((2-(trimethylsilyl)ethoxy)methyl)-1H-benzo[d]imidazol-2-yl)((R)-3,3-difluorocyclohexyl)methyl)-4-methyl-1,2,5-oxadiazole-3-carboxamide and N—((S)-(6-((R)-cyclopropyl(4,4,4-trifluorobutanamido)methyl)-1-((2-(trimethylsilyl)ethoxy)methyl)-1H-benzo[d]imidazol-2-yl)((R)-3,3-difluorocyclohexyl)methyl)-4-methyl-1,2,5-oxadiazole-3-carboxamide

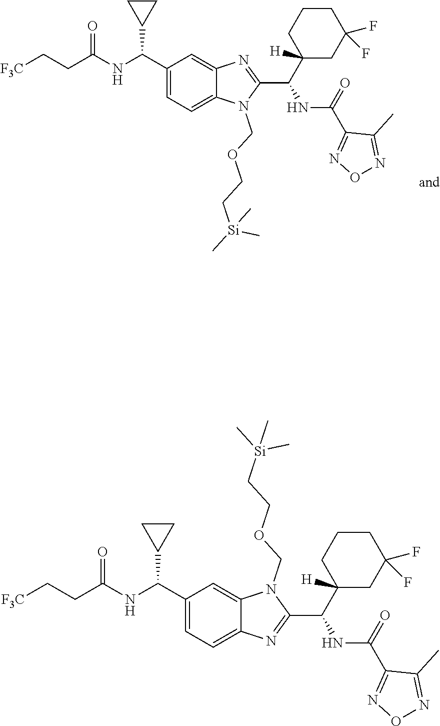 [SMILES: Cc1nonc1C(=O)N[C@H](c1nc2cc([C@H](NC(=O)CCC(F)(F)F)C3CC3)ccc2n1COCC[Si](C)(C)C)[C@@H]1CCCC(F)(F)C1 ; Cc1nonc1C(=O)N[C@H](c1nc2ccc([C@H](NC(=O)CCC(F)(F)F)C3CC3)cc2n1COCC[Si](C)(C)C)[C@@H]1CCCC(F)(F)C1]

A solution of N—((R)-(2-((S)-amino((R)-3,3-difluorocyclohexyl)methyl)-1-((2-(trimethylsilyl)ethoxy)methyl)-1H-benzo[d]imidazol-5-yl)(cyclopropyl)methyl)-4,4,4-trifluorobutanamide and N—((R)-(2-((S)-amino((R)-3,3-difluorocyclohexyl)methyl)-1-((2-(trimethylsilyl)ethoxy)methyl)-1H-benzo[d]imidazol-6-yl)(cyclopropyl)methyl)-4,4,4-trifluorobutanamide (75 mg, 0.13 mmol, Intermediate 397) and DIPEA (0.11 mL, 0.64 mmol) in DCM (4.7 mL) was cooled to 0° C. and treated with a solution of 4-methyl-1,2,5-oxadiazole-3-carbonyl chloride (56 mg, 0.38 mmol, Intermediate 79) in DCM (1 mL). The resulting solution was slowly warmed to rt over 2 h. The reaction mixture was poured over water and diluted with EtOAc. The layers were separated, and the aqueous phase was further extracted with EtOAc (2×5 mL). The combined organics were washed with brine, dried over anhydrous MgSO₄, filtered, and concentrated to dryness to provide the title compounds as a white solid.

### Intermediate 399

### N—((R)-1-(2-((S)—(((R)-tert-Butylsulfinyl)amino)((R)-3,3-difluorocyclohexyl)methyl)-1-((2-(trimethylsilyl)ethoxy)methyl)-1H-benzo[d]imidazol-5-yl)ethyl)-4,4,4-trifluorobutanamide and (R)—N—((S)—((R)-3,3-difluorocyclohexyl)(6-((R)-1-((3,3,3-trifluoropropyl)amino)ethyl)-1-((2-(trimethylsilyl)ethoxy)methyl)-1H-benzo[d]imidazol-2-yl)methyl)-2-methylpropane-2-sulfinamide

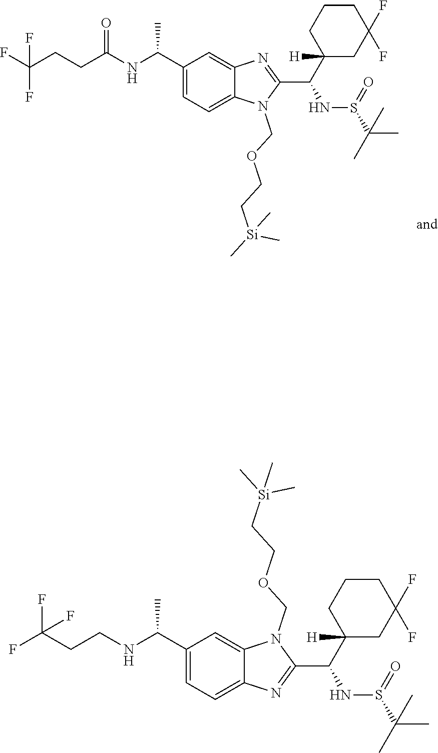 [SMILES: C[C@@H](NC(=O)CCC(F)(F)F)c1ccc2c(c1)nc([C@@H](N[S@](=O)C(C)(C)C)[C@@H]1CCCC(F)(F)C1)n2COCC[Si](C)(C)C ; C[C@@H](NCCC(F)(F)F)c1ccc2nc([C@@H](N[S@](=O)C(C)(C)C)[C@@H]3CCCC(F)(F)C3)n(COCC[Si](C)(C)C)c2c1]

A solution of (R)-4,4,4-trifluoro-N-(1-(1-((2-(trimethylsilyl)ethoxy)methyl)-1H-benzo[d]imidazol-5-yl)ethyl)butanamide and (R)-3,3,3-trifluoro-N-(1-(1-((2-(trimethylsilyl)ethoxy)methyl)-1H-benzo[d]imidazol-6-yl)ethyl)propan-1-amine (405 mg, 0.98 mmol, Intermediate 147) in THF (20 mL) was cooled to −78° C. and treated with n-BuLi in hexanes (1.2 mL, 3 mmol, 2.5 N). After 30 min, a solution of (R)—N-((E)-((R)-3,3-difluorocyclohexyl)methylene)-2-methylpropane-2-sulfinamide (375 mg, 1.5 mmol, Intermediate 136) in THF (20 mL) was added. The resulting mixture went from bright orange to dark red. After 4 h slowly warming to rt, the reaction was quenched with water and partitioned between saturated aqueous NH₄Cl and EtOAc/hexanes (5:1). The organic layer was dried over anhydrous Na₂SO₄, filtered, concentrated, and purified by silica gel chromatography (0-100% EtOAc/hexanes) to afford the mixture of title compounds.

### Intermediate 400

### N—((R)-1-(2-((S)-Amino((R)-3,3-difluorocyclohexyl)methyl)-1-((2-(trimethylsilyl)ethoxy)methyl)-1H-benzo[d]imidazol-5-yl)ethyl)-4,4,4-trifluorobutanamide hydrochloride and N—((R)-1-(2-((S)-amino((R)-3,3-difluorocyclohexyl)methyl)-1-((2-(trimethylsilyl)ethoxy)methyl)-1H-benzo[d]imidazol-6-yl)ethyl)-3,3,3-trifluoropropan-1-amine hydrochloride

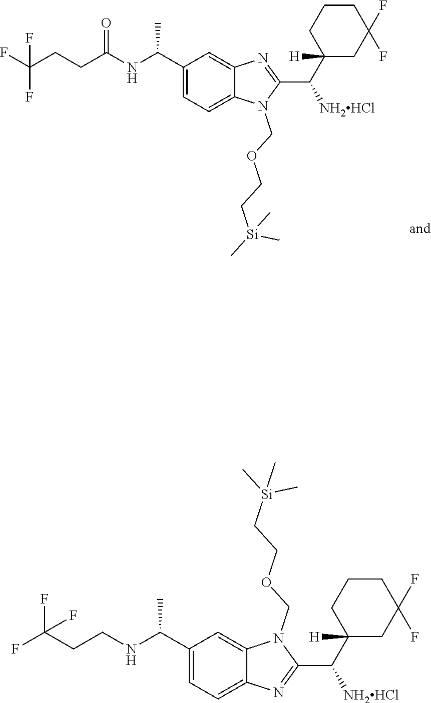

A solution of N—((R)-1-(2-((S)—(((R)-tert-butylsulfinyl)amino)((R)-3,3-difluorocyclohexyl)methyl)-1-((2-(trimethylsilyl)ethoxy)methyl)-1H-benzo[d]imidazol-5-yl)ethyl)-4,4,4-trifluorobutanamide and (R)—N—((S)—((R)-3,3-difluorocyclohexyl)(6-((R)-1-((3,3,3-trifluoropropyl)amino)ethyl)-1-((2-(trimethylsilyl)ethoxy)methyl)-1H-benzo[d]imidazol-2-yl)methyl)-2-methylpropane-2-sulfinamide (601 mg, 0.90 mmol, Intermediate 399) in DCM (9 mL) was treated with 2 N HCl in diethyl ether (10 mL, 20 mmol) at rt. The reaction immediately reached completion and was quenched with water. The aqueous phase was washed with DCM (2×10 mL, wash discarded) and then frozen and lyophilized. The title compounds were carried on without further purification.

### Intermediate 401

### N—((S)—((R)-3,3-Difluorocyclohexyl)(5-((R)-1-(4,4,4-trifluorobutanamido)ethyl)-1-((2-(trimethylsilyl)ethoxy)methyl)-1H-benzo[d]imidazol-2-yl)methyl)-4-methyl-1,2,5-oxadiazole-3-carboxamide and N—((S)—((R)-3,3-difluorocyclohexyl)(6-((R)-1-((3,3,3-trifluoropropyl)amino)ethyl)-1-((2-(trimethylsilyl)ethoxy)methyl)-1H-benzo[d]imidazol-2-yl)methyl)-4-methyl-1,2,5-oxadiazole-3-carboxamide

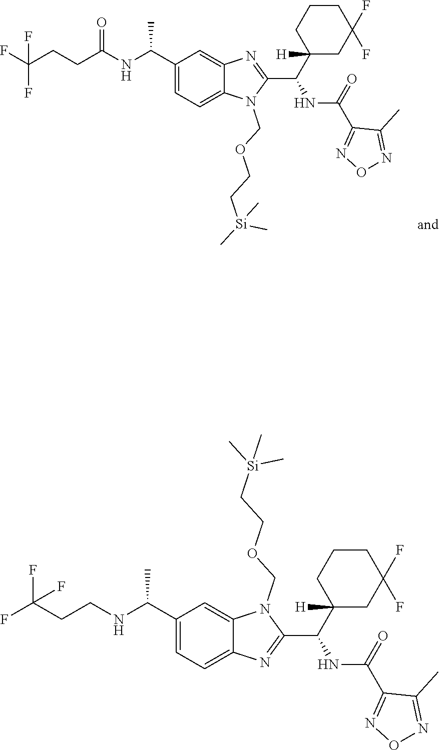 [SMILES: Cc1nonc1C(=O)N[C@H](c1nc2cc([C@@H](C)NC(=O)CCC(F)(F)F)ccc2n1COCC[Si](C)(C)C)[C@@H]1CCCC(F)(F)C1 ; Cc1nonc1C(=O)N[C@H](c1nc2ccc([C@@H](C)NCCC(F)(F)F)cc2n1COCC[Si](C)(C)C)[C@@H]1CCCC(F)(F)C1]

A solution of N—((R)-1-(2-((S′)-amino((R)-3,3-difluorocyclohexyl)methyl)-1-((2-(trimethylsilyl)ethoxy)methyl)-1H-benzo[d]imidazol-5-yl)ethyl)-4,4,4-trifluorobutanamide hydrochloride and N—((R)-1-(2-((S)-amino((R)-3,3-difluorocyclohexyl)methyl)-1-((2-(trimethylsilyl)ethoxy)methyl)-1H-benzo[d]imidazol-6-yl)ethyl)-3,3,3-trifluoropropan-1l-amine hydrochloride (127 mg, 0.21 mmol, Intermediate 400), in acetonitrile (2 mL) was treated with DIPEA (0.15 mL, 0.87 mmol) and 2,5-dioxopyrrolidin-1-yl 4-methyl-1,2,5-oxadiazole-3-carboxylate (582 mg, 0.26 mmol, Intermediate 80) and the resulting mixture was stirred at rt for 45 min. After that time, the reaction was quenched with water and concentrated to remove the acetonitrile. The aqueous mixture was then extracted with EtOAc and the organic layer dried over anhydrous Na₂SO₄, filtered, and concentrated to afford the mixture of title compounds as a white solid.

### Intermediate 402

### N—((S)—((R)-3,3-Difluorocyclohexyl)(5-((R)-1-(4,4,4-trifluorobutanamido)ethyl)-1-((2-(trimethylsilyl)ethoxy)methyl)-1H-benzo[d]imidazol-2-yl)methyl)-2-(3,3,3-trifluoropropyl)-2H-1,2,3-triazole-4-carboxamide and N—((S)—((R)-3,3-difluorocyclohexyl)(6-((R)-1-(4,4,4-trifluorobutanamido)ethyl)-1-((2-(trimethylsilyl)ethoxy)methyl)-1H-benzo[d]imidazol-2-yl)methyl)-2-(3,3,3-trifluoropropyl)-2H-1,2,3-tri azole-4-carboxamide

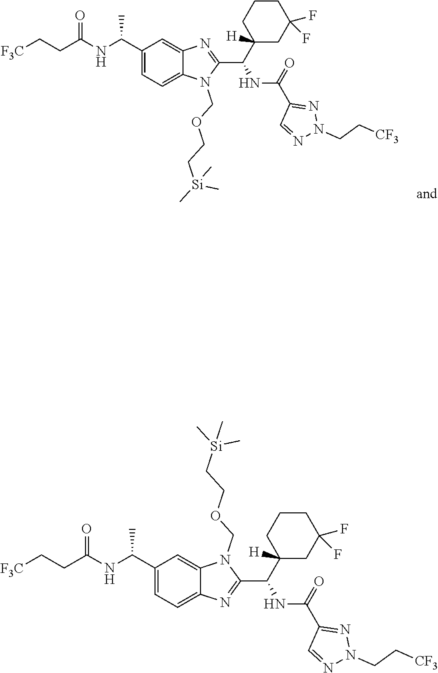 [SMILES: C[C@@H](NC(=O)CCC(F)(F)F)c1ccc2c(c1)nc([C@@H](NC(=O)c1cnn(CCC(F)(F)F)n1)[C@@H]1CCCC(F)(F)C1)n2COCC[Si](C)(C)C ; C[C@@H](NC(=O)CCC(F)(F)F)c1ccc2nc([C@@H](NC(=O)c3cnn(CCC(F)(F)F)n3)[C@@H]3CCCC(F)(F)C3)n(COCC[Si](C)(C)C)c2c1]

A vial was charged with N—((R)-1-(2-((S)-amino((R)-3,3-difluorocyclohexyl)methyl)-1-((2-(trimethylsilyl)ethoxy)methyl)-1H-benzo[d]imidazol-5-yl)ethyl)-4,4,4-trifluorobutanamide hydrochloride and N—((R)-1-(2-((S)-amino((R)-3,3-difluorocyclohexyl)methyl)-1-((2-(trimethylsilyl)ethoxy)methyl)-1H-benzo[d]imidazol-6-yl)ethyl)-3,3,3-trifluoropropan-1-amine hydrochloride (159 mg, 0.27 mmol, Intermediate 400), DMF (3 mL), HOBt (66 mg, 0.43 mmol), EDCI (88 mg, 0.46 mmol), DIPEA (0.4 mL, 2.3 mmol) and 2-(3,3,3-trifluoropropyl)-2H-1,2,3-triazole-4-carboxylic acid (93.3 mg, 0.45 mmol, Intermediate 7). The resulting mixture was stirred at 60° C. for 30 min. Then the reaction mixture was poured over water and diluted with EtOAc/hexanes. The layers were separated, and the aqueous phase was further extracted with EtOAc (2×5 mL). The combined organic layers were washed with brine, dried over anhydrous Na₂SO₄, filtered, and concentrated to dryness to afford the mixture of title compounds as a white solid.

### Intermediate 403

### N—((R)-(2-((S)—(((R)-tert-Butylsulfinyl)amino)((1R,5S,6r)-3,3-difluorobicyclo[3.1.0]hexan-6-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide

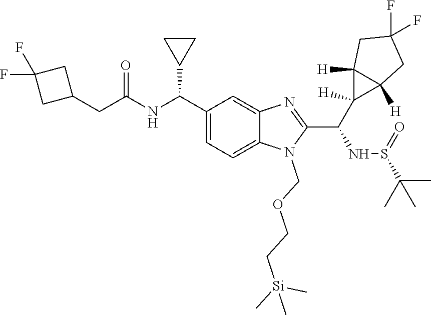 [SMILES: CC(C)(C)[S@@](=O)N[C@H](c1nc2cc([C@H](NC(=O)CC3CC(F)(F)C3)C3CC3)ccc2n1COCC[Si](C)(C)C)[C@H]1[C@@H]2CC(F)(F)C[C@@H]21]

The title compound was prepared as described for the synthesis of Intermediate 211, using (R)—N-((E)-((1R,5S,6r)-3,3-difluorobicyclo[3.1.0]hexan-6-yl)methylene)-2-methylpropane-2-sulfinamide (Intermediate 343) in place of (R,E)-2-methyl-N-(spiro[2.5]octan-6-ylmethylene)propane-2-sulfinamide. The crude product was purified by silica gel chromatography (0-10% MeOH/DCM) and basic preparative HPLC (Xtimate C18, 5 mm, 40×10 mm, 60-90% MeCN in water (0.05% NH₄OH)) to afford the title compound as a yellow solid.

### Intermediate 404

### N—((R)-(2-((S)-Amino((1R,5S,6r)-3,3-difluorobicyclo[3.1.0]hexan-6-yl)methyl)-1H-benzo[d]imidazol-5-yl)(cyclopropyl)methyl)-2-(3,3-difluorocyclobutyl)acetamide hydrochloride

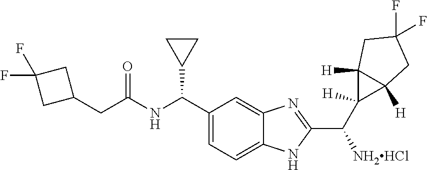

The title compound was prepared as described for the synthesis of Intermediate 361, using N—((R)-(2-((S)—(((R)-tert-butylsulfinyl)amino)((1R,5S,6r)-3,3-difluorobicyclo[3.1.0]hexan-6-yl)methyl)-1-((2-(trimethylsilyl)ethoxy)methyl)-1H-benzo[d]imidazol-5-yl)(cyclopropyl)methyl)-2-(3,3-difluorocyclobutyl)acetamide (Intermediate 403) in place of N—((R)-(2-((S)-1-(((R)-tert-butylsulfinyl)amino)-4,4-difluoro-3,3-dimethylbutyl)-1-((2-(trimethylsilyl)ethoxy)methyl)-1H-benzo[d]imidazol-6-yl)(cyclopropyl)methyl)-2-(3,3-difluorocyclobutyl)acetamide. The reaction mixture was concentrated to dryness and used without further purification.

### Intermediate 405

### 2-(3-Methoxyprop-1-en-2-yl)-4,4,5,5-tetramethyl-1,3,2-dioxaborolane

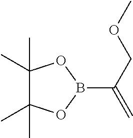

An oven dried round-bottom flask was charged with CuCl (176 mg, 1.78 mmol), sodium tert-butoxide (256 mg, 2.7 mmol), tri-tert-butylphosphonium tetrafluoroborate (618 mg, 2.13 mmol), and bis(pinacolato)diboron (5.86 g, 23.1 mmol) then vacuum purged and backfilled with N₂. In a separate flask, a mixture of MeOH (1.5 mL) and toluene (50 mL) was degassed by N₂ sparging for 5 min. Both flasks were cooled to 0° C., the solvent mixture was added to the reaction flask by cannula transfer. To the solution was added methyl propargyl ether (1.5 mL, 17.8 mmol). The reaction was allowed to warm to rt. After stirring for 5 h, MeOH (20 mL) was added and the mixture was filtered through Celite©. The filtrate was condensed to afford the title compound.

### Intermediate 406

### tert-Butyl (R)-(1-(5-bromo-1H-benzo[d]imidazol-2-yl)-2-((1,1,1-trifluoro-2-methylpropan-2-yl)oxy)ethyl)carbamate

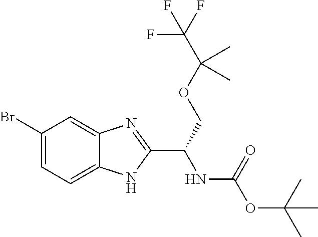 [SMILES: CC(C)(C)OC(=O)N[C@@H](COC(C)(C)C(F)(F)F)c1nc2cc(Br)ccc2[nH]1]

To a solution of (R)—N—((R)-1-(5-bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide and (R)—N—((R)-1-(6-bromo-1-((2-(trimethylsilyl)ethoxy)methyl)-1H-benzo[d]imidazol-2-yl)-2-((1,1,1-trifluoro-2-methylpropan-2-yl)oxy)ethyl)-2-methylpropane-2-sulfinamide (6040 mg, 10.1 mmol, Intermediate 282) in 1,4-dioxane (80 mL) and MeOH (20 mL) was added HCl (25 mL, 101 mmol, 4 M in 1,4-dioxane) and the reaction heated to 65° C. After 4 h, the mixture was cooled to rt, diluted with H₂O (100 mL) and washed with 4:1 EtOAc/hexanes (2×30 mL, wash discarded). The aqueous layer was made basic (pH>10) by the addition of Na₂CO₃ (2.2 g) then extracted with EtOAc (5×50 mL). The combined EtOAc extracts were washed with brine, dried over anhydrous Na₂SO₄, filtered, and concentrated. The residue was dissolved in 100 mL EtOAc then di-tert-butyl dicarbonate (2.2 g, 10.1 mmol) and DIPEA (1.9 mL, 11.1 mmol) were added. The reaction was stirred at rt for 16 h. H₂O (100 mL) was added and the layers were separated. The aqueous layer was extracted with EtOAc (3×50 mL) then the combined organic layers were washed with saturated aqueous ammonium chloride and brine, dried over anhydrous Na₂SO₄, filtered and concentrated. Purification by silica gel chromatography (10-70% (10% MeOH in EtOAc)/hexanes) provided the title compound.

### Intermediate 407

### tert-Butyl (R)-(1-(5-(3-methoxyprop-1-en-2-yl)-1H-benzo[d]imidazol-2-yl)-2-((1,1,1-trifluoro-2-methylpropan-2-yl)oxy)ethyl)carbamate

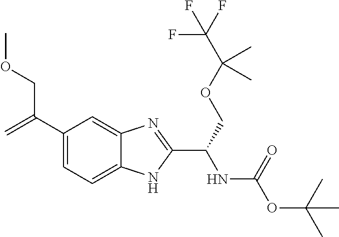 [SMILES: C=C(COC)c1ccc2[nH]c([C@H](COC(C)(C)C(F)(F)F)NC(=O)OC(C)(C)C)nc2c1]

To a solution of tert-butyl (R)-(1-(5-bromo-1H-benzo[d]imidazol-2-yl)-2-((1,1,1-trifluoro-2-methylpropan-2-yl)oxy)ethyl)carbamate (600 mg, 1.3 mmol, Intermediate 406) and 2-(3-methoxyprop-1-en-2-yl)-4,4,5,5-tetramethyl-1,3,2-dioxaborolane (1.02 g, 1.8 mmol, 35% purity, Intermediate 405) in 1,4-dioxane (12 mL) was added a solution of K₃PO₄ (885 mg, 3.86 mmol) in H₂O (2.4 mL). The mixture was degassed by N₂ sparging for 10 min then (2-dicyclohexylphosphino-2′,6′-diisopropoxy-1,1′-biphenyl)[2-(2′-amino-1,1′-biphenyl)]palladium(II) methanesulfonate (54 mg, 0.064 mmol) was added. The reaction was sealed under a stream of N₂ then warmed to 100° C. with microwave irradiation for 60 min. The mixture was concentrated, and the crude residue was partitioned between EtOAc and H₂O. The aqueous layer was extracted twice more with EtOAc then the combined organic layers were washed with brine, dried over anhydrous Na₂SO₄, filtered, and concentrated to afford the title compound that was used without further purification.

### Intermediate 408

### tert-Butyl (R)-(1-(5-(2-methoxyacetyl)-1H-benzo[d]imidazol-2-yl)-2-((1,1,1-trifluoro-2-methylpropan-2-yl)oxy)ethyl)carbamate

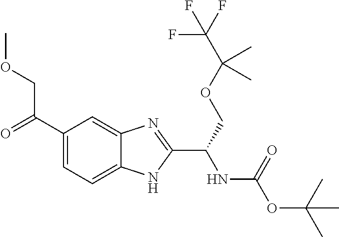 [SMILES: COCC(=O)c1ccc2[nH]c([C@H](COC(C)(C)C(F)(F)F)NC(=O)OC(C)(C)C)nc2c1]

To a solution of tert-butyl (R)-(1-(5-(3-methoxyprop-1-en-2-yl)-1H-benzo[d]imidazol-2-yl)-2-((1,1,1-trifluoro-2-methylpropan-2-yl)oxy)ethyl)carbamate (590 mg, 1.3 mmol, Intermediate 407) in 1,4-dioxane (4 mL) was added sodium periodate (1.1 g, 5.2 mmol) as a suspension in H₂O (4 mL) followed by potassium osmate dihydrate (47 mg, 0.13 mmol). The mixture was stirred at rt for 40 min then diluted with H₂O and extracted three times with EtOAc. The combined organics were washed with saturated aqueous sodium metabisulfite and brine, then dried over anhydrous MgSO₄, filtered and concentrated. Purification by silica gel chromatography (0-100% EtOAc/hexanes) provided the title compound.

### Intermediate 409

### tert-Butyl ((1R)-1-(5-(1-amino-2-methoxyethyl)-1H-benzo[d]imidazol-2-yl)-2-((1,1,1-trifluoro-2-methylpropan-2-yl)oxy)ethyl)carbamate

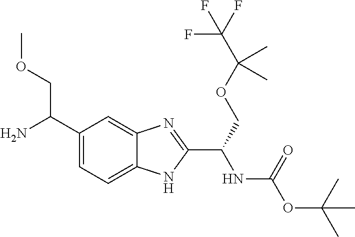 [SMILES: COCC(N)c1ccc2[nH]c([C@H](COC(C)(C)C(F)(F)F)NC(=O)OC(C)(C)C)nc2c1]

A vial was charged with tert-butyl (R)-(1-(5-(2-methoxyacetyl)-1H-benzo[d]imidazol-2-yl)-2-((1,1,1-trifluoro-2-methylpropan-2-yl)oxy)ethyl)carbamate (223 mg, 0.485 mmol, Intermediate 408), then NH₄OAc (750 mg, 9.7 mmol) in MeOH (2 mL) was added. The reaction was heated to 50° C. for 1 h then cooled to rt. Sodium cyanoborohydride (61 mg, 0.97 mmol) and acetic acid (28 μL, 0.49 mmol) were added and the reaction was stirred at rt for 72 h. The mixture was condensed then partitioned between EtOAc and H₂O. The aqueous layer was extracted twice more with EtOAc then the combined organic layers were washed with saturated aqueous sodium bicarbonate and brine, dried over anhydrous Na₂SO₄, filtered, and condensed to afford the title compound that was used without further purification.

### Intermediate 410

### tert-Butyl ((1R)-1-(5-(1-(2-(3,3-difluorocyclobutyl)acetamido)-2-methoxyethyl)-1H-benzo[d]imidazol-2-yl)-2-((1,1,1-trifluoro-2-methylpropan-2-yl)oxy)ethyl)carbamate

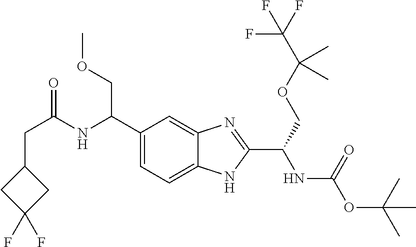 [SMILES: COCC(NC(=O)CC1CC(F)(F)C1)c1ccc2[nH]c([C@H](COC(C)(C)C(F)(F)F)NC(=O)OC(C)(C)C)nc2c1]

A solution of tert-butyl ((1R)-1-(5-(1-amino-2-methoxyethyl)-1H-benzo[d]imidazol-2-yl)-2-((1,1,1-trifluoro-2-methylpropan-2-yl)oxy)ethyl)carbamate (334 mg, 0.73 mmol, Intermediate 409), 2-(3,3-difluorocyclobutyl)acetic acid (120 mg, 0.80 mmol), DIPEA (0.2 mL, 1.2 mmol), and HOBt (126 mg, 0.80 mmol) in MeCN (10 mL) was heated to 45° C. and then EDCI (153 mg, 0.80 mmol) was added. The reaction was stirred at 45° C. for 30 min then quenched with H₂O and condensed. The residue was dissolved in EtOAc and washed with saturated aqueous ammonium chloride, saturated aqueous sodium bicarbonate and brine. The organic layer was dried over anhydrous Na₂SO₄, filtered, and concentrated. Purification by silica gel chromatography (5-100% (10% MeOH in EtOAc)/hexanes) provided the title compound.

### Intermediate 411

### N-(1-(2-((R)-1-Amino-2-((1,1,1-trifluoro-2-methylpropan-2-yl)oxy)ethyl)-1H-benzo[d]imidazol-5-yl)-2-methoxyethyl)-2-(3,3-difluorocyclobutyl)acetamide

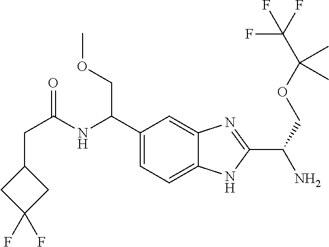 [SMILES: COCC(NC(=O)CC1CC(F)(F)C1)c1ccc2[nH]c([C@@H](N)COC(C)(C)C(F)(F)F)nc2c1]

A solution of tert-butyl ((1R)-1-(5-(1-(2-(3,3-difluorocyclobutyl)acetamido)-2-methoxyethyl)-1H-benzo[d]imidazol-2-yl)-2-((1,1,1-trifluoro-2-methylpropan-2-yl)oxy)ethyl)carbamate (178 mg, 0.3 mmol, Intermediate 410) in CH₂Cl₂ (0.5 mL) was treated with TFA (0.5 mL) and stirred at rt for 25 min. The mixture was diluted with EtOAc and neutralized with saturated aqueous sodium bicarbonate. The layers were separated, and the organic layer was washed with saturated aqueous sodium bicarbonate and brine then dried over anhydrous Na₂SO₄, filtered, and concentrated to afford the title compound that was used without further purification.

### Intermediate 412

### Ethyl 1-(ethyl-d₅)-1H-pyrazole-5-carboxylate

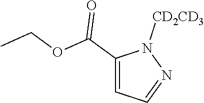

To a mixture of ethyl 1H-pyrazole-3-carboxylate (6.2 g, 44 mmol), K₂CO₃ (9.1 g, 66 mmol) and DMF (55 mL) was added bromoethane-d₅ (5.0 g, 44 mmol) and the resulting mixture was stirred at rt for 15 h. The reaction mixture was partitioned between EtOAc and water. The layers were separated, and the aqueous layer was further extracted with EtOAc. The organic layers were combined, washed with brine, dried over anhydrous Na₂SO₄, filtered, and concentrated to dryness. This residue was purified by silica gel chromatography (10-60% EtOAc/hexanes) to afford the title compound as the first eluting fraction, as a colorless oil.

### Intermediate 413

### 1-(Ethyl-d₅)-1H-pyrazole-5-carboxylic acid

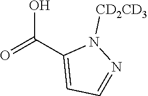

The title compound was prepared as described for the synthesis of Intermediate 7, using ethyl 1-(ethyl-d₅)-1H-pyrazole-5-carboxylate (Intermediate 412) in place of methyl 2-(3,3,3-trifluoropropyl)-2H-1,2,3-triazole-4-carboxylate to provide the title compound as a white solid.

### Intermediate 414

### Ethyl 1-(cyclobutylmethyl)-1H-1,2,3-triazole-5-carboxylate

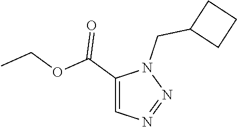

The title compound was prepared as described for the synthesis of Intermediate 89. Ethyl 1-(cyclobutylmethyl)-1H-1,2,3-triazole-5-carboxylate was the third eluting isomer, isolated as a clear colorless oil.

### Intermediate 415

### 1-(Cyclobutylmethyl)-1H-1,2,3-triazole-5-carboxylic acid

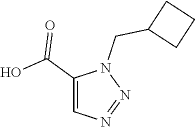 [SMILES: O=C(O)c1cnnn1CC1CCC1]

The title compound was prepared as described for the synthesis of Intermediate 90, using ethyl 1-(cyclobutylmethyl)-1H-1,2,3-triazole-5-carboxylate (Intermediate 414) in place of ethyl 2-(cyclobutylmethyl)-2H-1,2,3-triazole-4-carboxylate to provide the title compound as a white solid.

### Intermediate 416

### (R,E)-2-Methyl-N-((1-((2-(trimethylsilyl)ethoxy)methyl)-1H-benzo[d]imidazol-5-yl)methylene)propane-2-sulfinamide

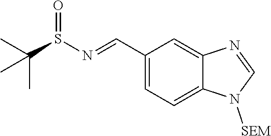 [SMILES: CC(C)(C)[S@@](=O)/N=C/c1ccc2c(c1)ncn2COCC[Si](C)(C)C]

### Intermediate 417

### (R,E)-2-Methyl-N-((1-((2-(trimethylsilyl)ethoxy)methyl)-1H-benzo[d]imidazol-6-yl)methylene)propane-2-sulfinamide

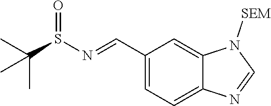 [SMILES: CC(C)(C)[S@@](=O)/N=C/c1ccc2ncn(COCC[Si](C)(C)C)c2c1]

1-((2-(Trimethylsilyl)ethoxy)methyl)-1H-benzo[d]imidazole-5-carbaldehyde and 1-((2-(trimethylsilyl)ethoxy)methyl)-1H-benzo[d]imidazole-6-carbaldehyde (14.4 g crude, 52.1 mmol, Intermediate 237), (R)-2-methylpropane-2-sulfinamide (7.58 g, 62.5 mmol) and DCM (300 mL) were combined followed by the addition of Cs₂CO₃ (25.5 g, 78.3 mmol). The resulting mixture was stirred at 25° C. overnight. After that time, the reaction mixture was filtered through a pad of Celite® and the filter cake was washed with DCM (100 mL). The filtrate was washed with water (500 mL), dried over anhydrous Na₂SO₄, filtered, and concentrated under reduced pressure. The resultant yellow oil was purified by silica gel chromatography (0-3% MeOH/DCM) to give a mixture of the two title compounds that were separated by preparative HPLC (YMC Exphere C18, 250×50 mm, 10 μm column, (40-69% (v/v) CH₃CN in H₂O with 0.04% NH₄OH and 10 mM NH₄HCO₃)) affording Intermediate 417 as the first eluting fraction and Intermediate 416 as the second eluting fraction.

### Intermediate 418

### (R)—N—((S)-(1-Cyanocyclopropyl)(1-((2-(trimethylsilyl)ethoxy)methyl)-1H-benzo[d]imidazol-6-yl)methyl)-2-methylpropane-2-sulfinamide

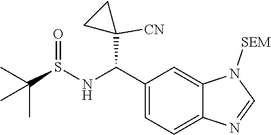 [SMILES: CC(C)(C)[S@@](=O)N[C@@H](c1ccc2ncn(COCC[Si](C)(C)C)c2c1)C1(C#N)CC1]

Cyclopropanecarbonitrile (1.41 g, 21.1 mmol) was dissolved in THF (50 mL) and cooled to −40° C. The resultant cold solution was charged with lithium magnesium 2,2,6,6-tetramethylpiperidin-1-ide dichloride (1 M in THF, 36.52 mL, 36.52 mmol) dropwise over 20 min, and allowed to stir at −40° C. for 3 h. A separate solution of (R,E)-2-methyl-N-((1-((2-(trimethylsilyl)ethoxy)methyl)-1H-benzo[d]imidazol-6-yl)methylene)propane-2-sulfinamide (4.00 g, 10.5 mmol, Intermediate 417) in THF (20 mL) was then added dropwise over 20 min, and the reaction was allowed to gradually warm to 25° C. and stir for 16 h. The reaction mixture was then treated with saturated aqueous NH₄Cl (50 mL). The biphasic mixture was then extracted with EtOAc (3×50 mL) and the combined organic layers were washed with brine (100 mL), dried over anhydrous Na₂SO₄, filtered, and concentrated under reduced pressure to afford a pale-yellow oil. The crude product was purified by silica gel chromatography (0-20% MeOH/DCM) and further separated by SFC using a chiral stationary phase (DAICEL CHIRALPAK AD column, 250×50 mm, 10 μm, mobile phase 80% CO₂ in EtOH (0.1% of 25% aqueous NH₃)) to afford the title compound, after lyophilization, as a white solid.

### Intermediate 419

### (S)-1-(Amino(1-((2-(trimethylsilyl)ethoxy)methyl)-1H-benzo[d]imidazol-6-yl)methyl)cyclopropane-1-carbonitrile hydrogen chloride

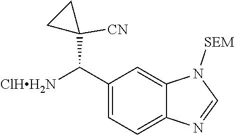

A 4 M solution of HCl in EtOAc (20 mL) was added to a solution of (R)—N—((S)-(1-cyanocyclopropyl)(1-((2-(trimethylsilyl)ethoxy)methyl)-1H-benzo[d]imidazol-6-yl)methyl)-2-methylpropane-2-sulfinamide (3.0 g, 6.7 mmol, Intermediate 418) in EtOAc (100 mL), and the resulting mixture was stirred for 1 h at 25° C. After this time, the solution was concentrated to under reduced pressure to a white solid that was used without further purification.

### Intermediate 420

### (S)-1-((1,3-Dioxoisoindolin-2-yl)(1-((2-(trimethylsilyl)ethoxy)methyl)-1H-benzo[d]imidazol-6-yl)methyl)cyclopropane-1-carbonitrile

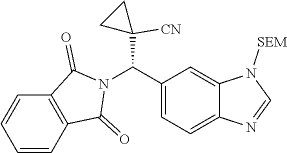 [SMILES: C[Si](C)(C)CCOCn1cnc2ccc([C@H](N3C(=O)c4ccccc4C3=O)C3(C#N)CC3)cc21]

TEA (2.80 mL, 20.1 mmol) was added to a solution of (S)-1-(amino(1-((2-(trimethylsilyl)ethoxy)methyl)-1H-benzo[d]imidazol-6-yl)methyl)cyclopropane-1-carbonitrile hydrogen chloride (2.54 g, 6.70 mmol, Intermediate 419) in toluene (100 mL) at 25° C. Isobenzofuran-1,3-dione (1.45 g, 10.1 mmol) was added followed by the connection of a dean-stark trap and reflux condenser to the reaction vessel, and the reaction was subsequently heated to 120° C. and stirred for 16 h. The solution was allowed to cool to 25° C., concentrated under reduced pressure, and purified by silica gel chromatography (0-50% EtOAc/petroleum ether) to afford the title compound as a yellow solid.

### Intermediate 421

### (R)-2-(((R)-1,1,1-Trifluoropropan-2-yl)oxy)propanoic acid

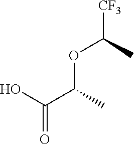

Sodium hydride (3.5 g, 88 mmol, 60% dispersion in mineral oil) was added in portions to a solution of (R)-1,1,1-trifluoropropan-2-ol (5.0 g, 44 mmol) in DMF (70 mL) at 0° C. The resultant mixture was stirred for 30 min at 0° C. A separate solution of (S)-2-bromopropanoic acid (6.0 g, 40 mmol) in DMF (5 mL) was added at 0° C. The resulting mixture was allowed to warm to rt and stir for 12 h. After this time, the reaction was poured into ice chilled water (100 mL) and extracted with MTBE (25 mL). The aqueous layer was acidified with 2 N aqueous HCl (15 mL) until the pH of the mixture was pH=5-6. This aqueous layer was extracted with MTBE (80 mL×3). The combined organic layers were washed with brine, dried over anhydrous Na₂SO₄, filtered, and concentrated under reduced pressure to afford the title compound as a yellow oil that was used without further purification.

### Intermediate 422

### (R)—N-Methoxy-N-methyl-2-(((R)-1,1,1-trifluoropropan-2-yl)oxy)propanamide

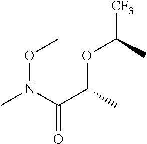

A round-bottom flask was charged with (R)-2-(((R)-1,1,1-trifluoropropan-2-yl)oxy)propanoic acid (7.6 g, 41 mmol, Intermediate 421), DMF (70 mL), HATU (20 g, 53 mmol), and DIPEA (18 mL, 102 mmol). The resulting mixture was stirred for 5 min at rt before N,O-dimethylhydroxylamine hydrochloride (6.0 g, 61 mmol) was added. The solution stirred for 12 h at rt, after which time, the reaction was quenched with water (30 mL) and diluted with MTBE (50 mL). The organic layer was separated, and the aqueous layer was extracted with MTBE (80 mL×2). The organic layers were combined, washed with water and brine, dried over anhydrous Na₂SO₄, filtered, and concentrated under reduced pressure. The product was purified by silica gel chromatography (9-17% EtOAc/petroleum ether) to afford the title compound as a yellow oil.

### Intermediate 423

### (R)-2-(((R)-1,1,1-Trifluoropropan-2-yl)oxy)propanal

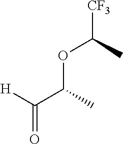

The title compound was prepared as described for the synthesis of Intermediate 322, using (R)—N-methoxy-N-methyl-2-(((R)-1,1,1-trifluoropropan-2-yl)oxy)propanamide (Intermediate 422) in place of N-methoxy-N-methyl-3-(2,2,2-trifluoroethyl)cyclobutane-1-carboxamide. The crude title compound was used directly in the next step without purification.

### Intermediate 424

### (R,E)-2-Methyl-N—((R)-2-(((R)-1,1,1-trifluoropropan-2-yl)oxy)propylidene)propane-2-sulfinamide

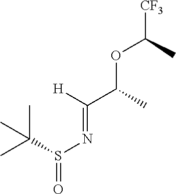 [SMILES: C[C@H](/C=N/[S@](=O)C(C)(C)C)O[C@H](C)C(F)(F)F]

A round-bottom flask was charged with (R)-2-(((R)-1,1,1-trifluoropropan-2-yl)oxy)propanal (4.0 g, 24 mmol, Intermediate 423), DCM (200 mL), CuSO₄ (15 g, 94 mmol), 4 Å molecular sieves (4.0 g), (R)-2-methylpropane-2-sulfinamide (5.7 g, 47 mmol), and PPTS (0.59 g, 2.4 mmol), and the resulting mixture was stirred at rt for 12 h. The suspension was filtered through Celite®*, the filter cake rinsed with EtOAc (100 mL), and the filtrate was concentrated under reduced pressure. The product was purified by silica gel chromatography (17-25% EtOAc/petroleum ether) to afford the title compound as a yellow oil.

### Intermediate 425

### (R)—N-((1R,2R)-1-(6-((S)-(1-Cyanocyclopropyl)(1,3-dioxoisoindolin-2-yl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

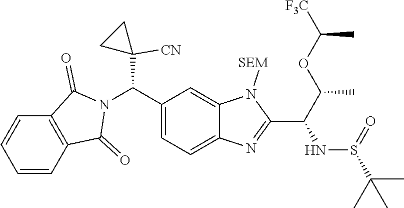 [SMILES: C[C@@H](O[C@H](C)C(F)(F)F)[C@H](N[S@](=O)C(C)(C)C)c1nc2ccc([C@H](N3C(=O)c4ccccc4C3=O)C3(C#N)CC3)cc2n1COCC[Si](C)(C)C]

The title compound was prepared as described for the synthesis of Intermediate 194, using (S)-1-((1,3-dioxoisoindolin-2-yl)(1-((2-(trimethylsilyl)ethoxy)methyl)-1H-benzo[d]imidazol-6-yl)methyl)cyclopropane-1-carbonitrile (Intermediate 420) in place of (R)-2-(cyclopropyl(1-((2-(trimethylsilyl)ethoxy)methyl)-1H-benzo[d]imidazol-5-yl)methyl)isoindoline-1,3-dione and (R,E)-2-methyl-N—((R)-2-(((R)-1,1,1-trifluoropropan-2-yl)oxy)propylidene)propane-2-sulfinamide (Intermediate 424) in place of (R,E)-2-methyl-N-(2-((1,1,1-trifluoro-2-methylpropan-2-yl)oxy)ethylidene)propane-2-sulfinamide. The crude title compound was purified by preparative TLC (9% MeOH/DCM) to afford the title compound as a yellow oil.

### Intermediate 426

### (R)—N-((1R,2R)-1-(6-((S)-Amino(1-cyanocyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

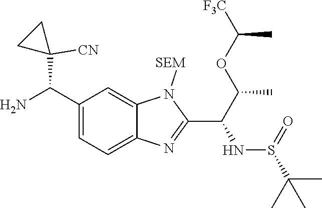 [SMILES: C[C@@H](O[C@H](C)C(F)(F)F)[C@H](N[S@](=O)C(C)(C)C)c1nc2ccc([C@H](N)C3(C#N)CC3)cc2n1COCC[Si](C)(C)C]

The title compound was prepared as described for the synthesis of Intermediate 265, using (R)—N-((1R,2R)-1-(6-((S)-(1-cyanocyclopropyl)(1,3-dioxoisoindolin-2-yl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (Intermediate 425) in place of (R)—N—((S)-1-(5-((R)-cyclopropyl(1,3-dioxoisoindolin-2-yl)methyl)-1-((2-(trimethylsilyl)ethoxy)methyl)-1H-benzo[d]imidazol-2-yl)-4,4,4-trifluoro-3,3-dimethylbutyl)-2-methylpropane-2-sulfinamide. The crude product was purified by silica gel chromatography (0-3% MeOH/DCM) to afford the title compound as a yellow oil.

### Intermediate 427

### (S)-4-Benzyl-3-(2-((S)-2,2-difluorocyclopropyl)acetyl)oxazolidin-2-one

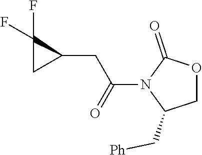 [SMILES: O=C(C[C@H]1CC1(F)F)N1C(=O)OC[C@@H]1Cc1ccccc1]

A round-bottom flask was charged with (S)-4-benzyloxazolidin-2-one (1.43 g, 8.08 mmol), DMAP (1.08 g, 8.82 mmol), and DCM (20 mL). The solution was stirred for 5 min at 25° C. prior to the sequential addition of 2-(2,2-difluorocyclopropyl)acetic acid (1.00 g, 7.35 mmol) and EDCI (1.48 g, 7.72 mmol). The reaction was then stirred for 16 h at 25° C., after which time, the reaction was diluted with DCM (20 mL) and washed with citric acid (50 mL), saturated aqueous NaHCO₃ (50 mL), and brine (50 mL). The organic layer was dried over anhydrous Na₂SO₄, filtered, and concentrated under reduced pressure to provide (S)-4-benzyl-3-(2-((R,S)-2,2-difluorocyclopropyl)acetyl)oxazolidin-2-one. These diastereomers were purified and separated by silica gel chromatography (0-40% EtOAc/hexanes) to afford the title compound, (S)-4-benzyl-3-(2-((S)-2,2-difluorocyclopropyl)acetyl)oxazolidin-2-one, as the second eluting fraction. The stereochemistry of this compound was confirmed by X-ray crystallography.

### Intermediate 428

### (S)-2-(2,2-Difluorocyclopropyl)acetic acid

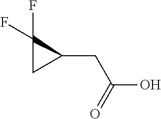

A round-bottom flask was charged with (S)-4-benzyl-3-(2-((S)-2,2-difluorocyclopropyl)acetyl)oxazolidin-2-one (100 mg, 0.339 mmol, Intermediate 427) and THF (6 mL). The solution was cooled to 0° C. and had 30% H₂O₂ (0.173 mL, 1.69 mmol) and LiOH·H₂O (28.4 mg, 0.677 mmol) in water (2 mL) sequentially added. The reaction was stirred for 1 h at 0° C., after which time the solution was poured into saturated aqueous NaHCO₃ (6 mL). The biphasic solution was washed with DCM (2×5 mL) and the combined organic layers were extracted with saturated aqueous NaHCO₃ (10 mL). The combined aqueous layers were acidified with 6 M aqueous HCl to pH=1 and extracted with MTBE (3×10 mL). The combined organic layers were dried over anhydrous Na₂SO₄, filtered, and concentrated under reduced pressure to afford the title compound as a colorless oil.

### Intermediate 429

### N—((S)-(2-((1R,2R)-1-(((R)-tert-Butylsulfinyl)amino)-2-(((R)-1,1,1-trifluoropropan-2-yl)oxy)propyl)-1-((2-(trimethylsilyl)ethoxy)methyl)-1H-benzo[d]imidazol-6-yl)(1-cyanocyclopropyl)methyl)-2-((S)-2,2-difluorocyclopropyl)acetamide

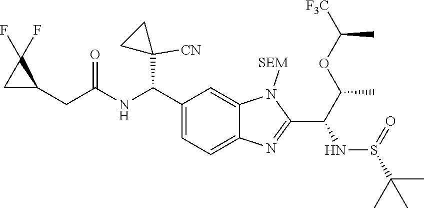 [SMILES: C[C@@H](O[C@H](C)C(F)(F)F)[C@H](N[S@](=O)C(C)(C)C)c1nc2ccc([C@H](NC(=O)C[C@H]3CC3(F)F)C3(C#N)CC3)cc2n1COCC[Si](C)(C)C]

A flask was charged with (S)-2-(2,2-difluorocyclopropyl)acetic acid (42 mg, 0.31 mmol, Intermediate 428), DCM (8 mL), HATU (150 mg, 0.39 mmol), and DIPEA (0.14 mL, 0.78 mmol). The resulting mixture was stirred for 10 min at rt. Then, (R)—N-((1R,2R)-1-(6-((S)-amino(1-cyanocyclopropyl)methyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (160 mg, 0.26 mmol, Intermediate 426) was added and the reaction stirred for 3 h at 25° C. After that time, the reaction was quenched with water (20 mL) and extracted with DCM (3×15 mL). The combined organic layers were washed with brine (30 mL), dried over anhydrous MgSO₄, filtered, and concentrated under reduced pressure to give a yellow oil. This oil was purified by silica gel chromatography (0-5% MeOH/DCM) to afford the title compound as a yellow oil.

### Intermediate 430

### N—((S)-(2-((1R,2R)-1-Amino-2-(((R)-1,1,1-trifluoropropan-2-yl)oxy)propyl)-1H-benzo[d]imidazol-6-yl)(1-cyanocyclopropyl)methyl)-2-((S)-2,2-difluorocyclopropyl)acetamide

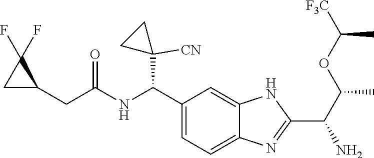 [SMILES: C[C@@H](O[C@H](C)C(F)(F)F)[C@H](N)c1nc2ccc([C@H](NC(=O)C[C@H]3CC3(F)F)C3(C#N)CC3)cc2[nH]1]

A flask was charged with N—((S)-(2-((1R,2R)-1-(((R)-tert-butylsulfinyl)amino)-2-(((R)-1,1,1-trifluoropropan-2-yl)oxy)propyl)-1-((2-(trimethylsilyl)ethoxy)methyl)-1H-benzo[d]imidazol-6-yl)(1-cyanocyclopropyl)methyl)-2-((S)-2,2-difluorocyclopropyl)acetamide (140 mg, 0.19 mmol, Intermediate 429), 1,4-dioxane (2 mL) and HCl (1 mL, 4 mmol, 4 M in 1,4-dioxane). The reaction was stirred at 55° C. for 2 h. After that time, the reaction was quenched with saturated aqueous NaHCO₃ (30 mL) and extracted with DCM (2×30 mL). The combined organic extracts were washed with brine, dried over anhydrous Na₂SO₄, filtered, and concentrated under reduced pressure to afford the title compound as a yellow oil.

### Intermediate 431

### 2-(4-Fluoro-3-nitrophenyl)-1,3-dioxolane

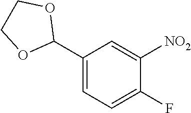 [SMILES: O=[N+]([O-])c1cc(C2OCCO2)ccc1F]

A mixture of 4-fluoro-3-nitrobenzaldehyde (450 g, 2.66 mol), ethylene glycol (446 mL, 7.98 mol) and TsOH (9.16 g, 53.2 mmol) in toluene (3 L) was purged with N₂ three times, and then the mixture was heated at 110° C. for 12 h. The reaction mixture was diluted with H₂O (15 L) and extracted with EtOAc (3×5 L). The combined organic layers were washed with brine (5 L), dried over anhydrous Na₂SO₄, filtered and concentrated to dryness. The material was triturated with petroleum ether (2 L) at 20-25° C. for 30 min to provide the title compound as a yellow solid (90% yield).

### Intermediate 432

### N-(4-(1,3-Dioxolan-2-yl)-2-nitrophenyl)formamide

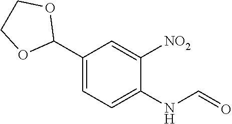 [SMILES: O=CNc1ccc(C2OCCO2)cc1[N+](=O)[O-]]

To a mixture of t-BuOK (2.62 kg, 23.3 mol) in NMP (20 L) at 0° C. was added formamide (1.86 L, 46.6 mol) followed by a solution of 2-(4-fluoro-3-nitrophenyl)-1,3-dioxolane (1990 g, 9.34 mol, Intermediate 431) in NMP (5 L) dropwise over 30 min. The resulting mixture was stirred at 0° C. for 30 min and then quenched by the addition of saturated aqueous NH₄CL (70 L) at 0° C. The mixture was extracted with EtOAc (2×15 L). The organic layers were combined, washed with brine (15 L), dried over anhydrous Na₂SO₄, filtered and concentrated to dryness. The residue was triturated with MTBE (6 L) at 20-25° C. for 30 min to provide the title compound as a yellow solid (71% yield).

### Intermediate 433

### N-(4-(1,3-Dioxolan-2-yl)-2-nitrophenyl)-N-((2-(trimethylsilyl)ethoxy)methyl)formamide

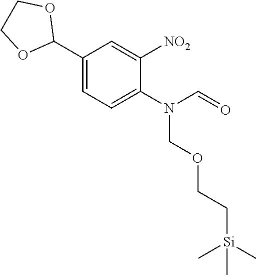 [SMILES: C[Si](C)(C)CCOCN(C=O)c1ccc(C2OCCO2)cc1[N+](=O)[O-]]

To a solution of N-(4-(1,3-dioxolan-2-yl)-2-nitrophenyl)formamide (1600 g, 6.72 mol, Intermediate 432) in THF (16 L) at −10° C. was added t-BuOK (979 g, 8.73 mol) and the resulting mixture was stirred at −10° C. for 30 min. Then, SEM-Cl (2.02 L, 11.4 mol) was added dropwise and the mixture was allowed to warm to 0° C. and stirred at 0° C. for 1 h. The reaction mixture was quenched by the addition of saturated aqueous NH₄Cl (20 L) and then extracted with EtOAc (3×5 L). The combined organic layers were washed with brine (5 L), dried over anhydrous Na₂SO₄, filtered and concentrated to dryness to provide the title compound as a brown oil which was used without further purification.

### Intermediate 434

### 1-((2-(Trimethylsilyl)ethoxy)methyl)-1H-benzo[d]imidazole-5-carbaldehyde

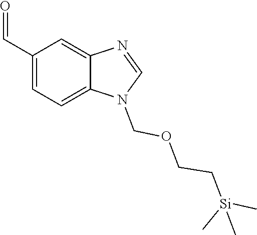 [SMILES: C[Si](C)(C)CCOCn1cnc2cc(C=O)ccc21]

A mixture of N-(4-(1,3-dioxolan-2-yl)-2-nitrophenyl)-N-((2-(trimethylsilyl)ethoxy)methyl)formamide (73.0 g, 0.198 mol, Intermediate 433), Fe (14.4 g, 0.257 mol) and AcOH (300 mL, 5.24 mol) in EtOH (730 mL) was heated at 80° C. for 16 h. The mixture was cooled to rt and concentrated to dryness. The residue was dissolved in EtOAc (1 L) and filtered. The filtrate was then washed sequentially with water (5×1 L), saturated aqueous Na₂CO₃ (1 L) and brine (1 L), dried over anhydrous Na₂SO₄, filtered and concentrated to dryness to provide the title compound as a brown oil which was used without further purification.

### Intermediate 435

### (R,E)-2,4,6-Trimethyl-N-((1-((2-(trimethylsilyl)ethoxy)methyl)-1H-benzo[d]imidazol-5-yl)methylene)benzenesulfinamide

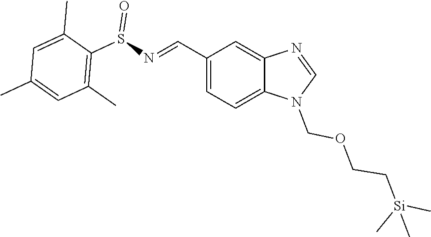

A mixture of 1-((2-(trimethylsilyl)ethoxy)methyl)-1H-benzo[d]imidazole-5-carbaldehyde (2.0 g, 7.2 mmol, Intermediate 434), (R)-2,4,6-trimethylbenzenesulfinamide (1.59 g, 8.68 mmol), and Cs₂CO₃ (2.83 g, 8.68 mmol) in DCM (20 mL) was stirred at rt overnight. The reaction mixture was then concentrated to dryness to yield a yellow oil. This oil was then diluted with H₂O (30 mL) and extracted with EtOAc (50 mL×3). The organic layers were combined, washed with brine (50 mL), dried over anhydrous Na₂SO₄, filtered, and concentrated to dryness to afford a yellow oil. The residue was purified by silica gel chromatography (0-60% EtOAc/petroleum ether) to provide the title compound as a yellow solid (78% yield).

### Intermediate 436

### 2-Cyclopropoxyacetic acid

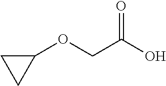

To a 0° C. solution of cyclopropanol (4.00 g, 68.9 mmol) in THF (75 mL) was added NaH (5.51 g, 138 mmol, 60% dispersion in mineral oil) portion-wise such that the temperature of the reaction didn't exceed 10° C. The resulting mixture was stirred at 0° C. for 5 min then the ice-water bath was removed and the reaction was stirred at rt for 2 h. Then, the mixture was cooled to 0° C. in an ice-water bath and a solution of 2-bromoacetic acid (7.66 g, 55.1 mmol) in THF (5 mL) was added dropwise over 10 min. The ice-water bath was removed and the mixture was stirred at rt for 12 h. The reaction mixture was cooled to 0° C. in an ice-water bath and quenched by the slow addition of water (100 mL). The mixture was concentrated to remove THE and then the aqueous layer was washed with DCM (3×100 mL). The pH of the aqueous phase was adjusted to pH 1-2 by the addition of 1 M aqueous HCl, and then the mixture was extracted with EtOAc (3×100 mL). The EtOAc layers were combined, washed with brine (100 mL), dried over anhydrous Na₂SO₄, filtered and concentrated to dryness to provide the title compound as a brown oil that was used without further purification.

### Intermediate 437

### 1,3-Dioxoisoindolin-2-yl 2-cyclopropoxyacetate

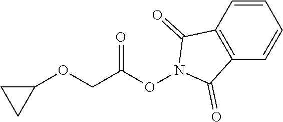 [SMILES: O=C(COC1CC1)ON1C(=O)c2ccccc2C1=O]

To a solution of 2-cyclopropylacetic acid (5.0 g, 43 mmol, Intermediate 436) in EtOAc (36 mL) was added 1-propanephosphonic anhydride (26 mL, 43 mmol, 50% in EtOAc). The reaction was stirred for 2 min at rt then 2-hydroxyisoindoline-1,3-dione (5.8 g, 36 mmol) was added. The resulting mixture was stirred at rt for 5 h then diluted with EtOAc. The mixture was washed sequentially with 1 M aqueous HCl, half saturated aqueous NaHCO₃, and brine, then dried over anhydrous Na₂SO₄, filtered, and concentrated to dryness to afford the title compound (62% yield).

### Intermediate 438

### (R)—N—((S)-2-Cyclopropoxy-1-(1-((2-(trimethylsilyl)ethoxy)methyl)-1H-benzo[d]imidazol-5-yl)ethyl)-2,4,6-trimethylbenzenesulfinamide

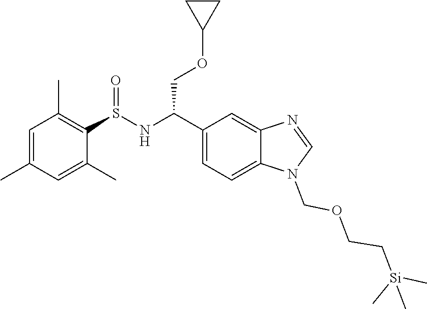

A mixture of (R,E)-2,4,6-trimethyl-N-((1-((2-(trimethylsilyl)ethoxy)methyl)-1H-benzo[d]imidazol-5-yl)methylene)benzenesulfinamide (400 mg, 0.661 mmol, Intermediate 435), 1,3-dioxoisoindolin-2-yl 2-cyclopropoxyacetate (518 mg, 1.98 mmol, Intermediate 437), diethyl 2,6-dimethyl-1,4-dihydropyridine-3,5-dicarboxylate (502 mg, 1.98 mmol) and DIPEA (0.23 mL, 1.3 mmol) in DMSO (10 mL) was sparged with argon for 10 min and then irradiated with 450 nm light (100% LED intensity, max rpm fan) for 6 h. After this time, additional 1,3-dioxoisoindolin-2-yl 2-cyclopropoxyacetate (400 mg, 1.53 mmol, Intermediate 437) and diethyl 2,6-dimethyl-1,4-dihydropyridine-3,5-dicarboxylate (400 mg, 1.58 mmol) were added. The mixture was sparged with argon for 10 min and irradiated with 450 nm light (100% LED intensity, max rpm fan) for 6 h. The reaction mixture was then diluted with H₂O (50 mL) and extracted with EtOAc (50 mL×4). The combined organic extracts were washed sequentially with H₂O (50 mL×2) and brine (50 mL×2), dried over anhydrous Na₂SO₄, filtered, and concentrated to dryness. The residue was dissolved in DCM (50 mL), washed with n-hexanes (250 mL), filtered, and concentrated to dryness to afford a yellow oil. The oil was then subjected to silica gel chromatography (0-70% EtOAc/petroleum ether) to provide the title compound as a light-yellow oil (35% yield).

### Intermediate 439

### (S)-2-Cyclopropoxy-1-(1-((2-(trimethylsilyl)ethoxy)methyl)-1H-benzo[d]imidazol-5-yl)ethan-1-amine

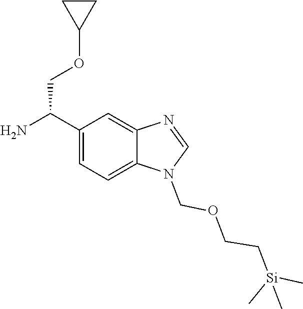 [SMILES: C[Si](C)(C)CCOCn1cnc2cc([C@H](N)COC3CC3)ccc21]

To a solution of (R)—N—((S)-2-cyclopropoxy-1-(1-((2-(trimethylsilyl)ethoxy)methyl)-1H-benzo[d]imidazol-5-yl)ethyl)-2,4,6-trimethylbenzenesulfinamide (100 mg, 0.195 mmol, Intermediate 438) in EtOAc (1.0 mL) was added HCl (0.195 mL, 0.78 mmol, 4 M in 1,4-dioxane) dropwise over 1 min. The resulting mixture was stirred at rt for 30 min. The mixture was diluted with H₂O and extracted with EtOAc (10 mL×3). The combined organic phases were washed with aqueous HCl (2 M, 5 mL×2). The pH of the aqueous phase was adjusted to pH 8 by the addition of NaHCO₃ (saturated, aqueous) and then extracted with EtOAc (20 mL×2). The combined organic extracts were washed with brine (50 mL), dried over anhydrous Na₂SO₄, filtered, and concentrated to dryness to afford the title compound as a light-yellow oil, that was used without further purification.

### Intermediate 440

### (S)-2-(2-Cyclopropoxy-1-(1-((2-(trimethylsilyl)ethoxy)methyl)-1H-benzo[d]imidazol-5-yl)ethyl)isoindoline-1,3-dione

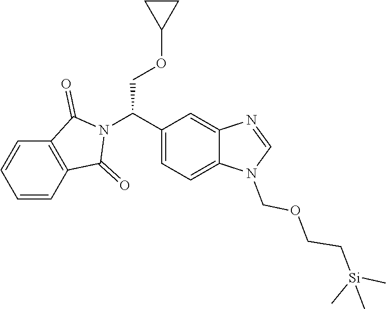 [SMILES: C[Si](C)(C)CCOCn1cnc2cc([C@@H](COC3CC3)N3C(=O)c4ccccc4C3=O)ccc21]

A mixture of (S)-2-cyclopropoxy-1-(1-((2-(trimethylsilyl)ethoxy)methyl)-1H-benzo[d]imidazol-5-yl)ethan-1-amine (885 mg, 2.55 mmol, Intermediate 439), ethyl 1,3-dioxoisoindoline-2-carboxylate (623 mg, 2.84 mmol) and DIPEA (1.48 mL, 8.49 mmol) in THF (10 mL) was heated at 75° C. for 48 h. Water (50 mL) was added and the mixture was extracted with EtOAc (100 mL×3). The organic extracts were combined, washed with brine (100 mL), dried over anhydrous Na₂SO₄, filtered, and concentrated to dryness to afford a yellow oil. This oil was subjected to silica gel chromatography (0-50% EtOAc/petroleum ether) to provide the title compound as a yellow oil (86% yield).

### Intermediate 441

### (R)-2-((1,1,1-Trifluoro-2-methylpropan-2-yl)oxy)propanoic acid

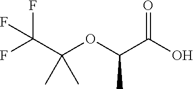

A solution of 1,1,1-trifluoro-2-methylpropan-2-ol (5.00 g, 39.0 mmol) in DMF (40 mL) was cooled to 0° C. in an ice-water bath and then NaH (3.12 g, 78.1 mmol, 60% dispersion in mineral oil) was added, the ice-water bath was removed, and the resulting solution was stirred at 25° C. for 1.5 h. After this time, the reaction was cooled to 0° C. and (S)-2-bromopropanoic acid (5.37 g, 35.1 mmol) was added followed by DMF (20 mL). The ice-water bath was removed and the solution was stirred at 25° C. for 12 h. The reaction mixture was poured into H₂O (60 mL) and the mixture was extracted with CH₂Cl₂ (40 mL×2). The pH of the aqueous layer was adjusted to pH 1-2 by the addition of aqueous HCl (1 N, 10 mL). The aqueous solution was then extracted with CH₂Cl₂ (50 mL×3). The organic layers were combined, washed with brine (200 mL×2), dried over anhydrous Na₂SO₄, filtered, and concentrated to dryness to afford the title compound as a yellow oil, which was used without further purification.

### Intermediate 442

### (R)—N-Methoxy-N-methyl-2-((1,1,1-trifluoro-2-methylpropan-2-yl)oxy)propanamide

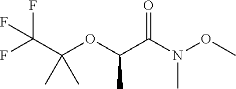 [SMILES: CON(C)C(=O)[C@@H](C)OC(C)(C)C(F)(F)F]

A mixture of (R)-2-((1,1,1-trifluoro-2-methylpropan-2-yl)oxy)propanoic acid (19 g, 19 mmol, Intermediate 441), HATU (47.0 g, 124 mmol), DIPEA (57.9 mL, 333 mmol) and N,O-dimethylhydroxylamine hydrochloride (13.9 g, 143 mmol) in DCM (50 mL) was stirred at rt for 5 h. The reaction mixture was then washed with brine (100 mL×2), dried over anhydrous Na₂SO₄, filtered, and concentrated to dryness to afford a yellow oil. The oil was then subjected to silica gel chromatography (0-35% EtOAc/petroleum ether) to provide the title compound (65% yield).

### Intermediate 443

### (R)-2-((1,1,1-Trifluoro-2-methylpropan-2-yl)oxy)propanal

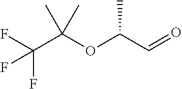

(R)—N-Methoxy-N-methyl-2-((1,1,1-trifluoro-2-methylpropan-2-yl)oxy)propanamide (3.4 g, 14 mmol, Intermediate 442) was added dropwise over 30 min to a solution of DIBAL-H (1 M in toluene, 30.8 mL, 30.8 mmol) which was cooled to −70° C. in a dry ice/EtOH bath. The resulting mixture was stirred at −70° C. for 1 h. Then, the reaction mixture was warmed to −50° C. and saturated aqueous potassium sodium tartrate (200 mL) was added over 30 min. The resulting mixture was stirred for 1 h at 25° C., then was extracted with CH₂Cl₂ (300 mL×3). The organic layers were combined, washed with brine (800 mL×2), dried over anhydrous MgSO₄, filtered, and concentrated to dryness to provide the title compound as a yellow oil, which was used without further purification.

### Intermediate 444

### (R)-2-Methyl-N—((R,E)-2-((1,1,1-trifluoro-2-methylpropan-2-yl)oxy)propylidene)propane-2-sulfinamide

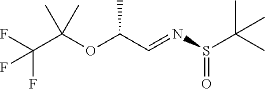 [SMILES: C[C@H](/C=N/[S@](=O)C(C)(C)C)OC(C)(C)C(F)(F)F]

To a mixture of (R)-2-((1,1,1-trifluoro-2-methylpropan-2-yl)oxy)propanal (7.5 g, 41 mmol, Intermediate 443), CuSO₄ (19.5 g, 122 mmol), PPTS (1.02 g, 4.07 mmol) and 4 Å molecular sieves (8 g) in anhydrous DCM (300 mL) was added (R)-2-methylpropane-2-sulfinamide (7.40 g, 61.1 mmol). The resulting mixture was stirred at rt for 16 h. The mixture was filtered and the filtrate was diluted with H₂O (150 mL). This mixture was extracted with CH₂Cl₂ (200 mL×3) and the organic layers were combined and dried over anhydrous Na₂SO₄. The mixture was filtered and concentrated to dryness to afford a light-yellow oil. The oil was subjected to silica gel chromatography (0-7% EtOAc/petroleum ether) to provide the title compound as a light-yellow oil (3.4% yield).

### Intermediate 445

### (R)—N-((1R,2R)-1-(5-((S)-2-Cyclopropoxy-1-(1,3-dioxoisoindolin-2-yl)ethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide

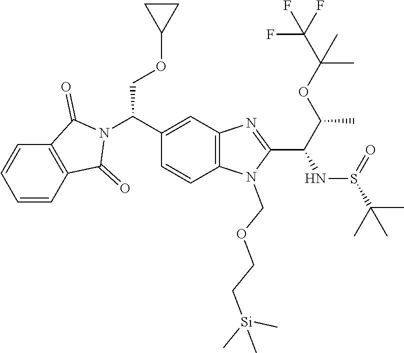 [SMILES: C[C@@H](OC(C)(C)C(F)(F)F)[C@H](N[S@](=O)C(C)(C)C)c1nc2cc([C@@H](COC3CC3)N3C(=O)c4ccccc4C3=O)ccc2n1COCC[Si](C)(C)C]

A solution of (S)-2-(2-cyclopropoxy-1-(1-((2-(trimethylsilyl)ethoxy)methyl)-1H-benzo[d]imidazol-5-yl)ethyl)isoindoline-1,3-dione (400 mg, 0.837 mmol, Intermediate 440) and (R)-2-methyl-N—((R,E)-2-((1,1,1-trifluoro-2-methylpropan-2-yl)oxy)propylidene)propane-2-sulfinamide (385 mg, 1.34 mmol, Intermediate 444) in THF (anhydrous, 30 mL) was cooled to −70° C. in a dry ice/EtOH bath and then LDA (2.9 mL, 2.9 mmol, 1.0 M in THF) was added dropwise over 25 min. The resulting mixture was stirred at −70° C. for 0.5 h and then quenched with a solution of AcOH (2% in THF, 3 mL). The mixture was stirred for 2 min at −70° C. before the dry ice/EtOH bath was removed and the reaction was allowed to warm to rt. The reaction mixture was diluted with H₂O (50 mL) and extracted with EtOAc (50 mL×4). The combined organic extracts were washed with brine (50 mL×2), dried over anhydrous MgSO₄, filtered, and concentrated to dryness to give a yellow oil. This oil was subjected to silica gel chromatography (0-0.2% MeOH/DCM) to afford the title compound (41% yield).

### Intermediate 446

### (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide

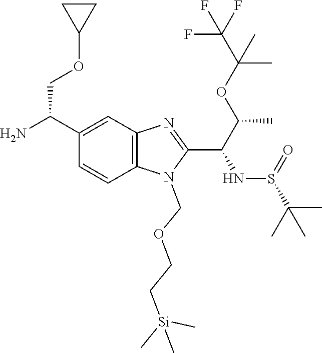 [SMILES: C[C@@H](OC(C)(C)C(F)(F)F)[C@H](N[S@](=O)C(C)(C)C)c1nc2cc([C@H](N)COC3CC3)ccc2n1COCC[Si](C)(C)C]

To a solution of (R)—N-((1R,2R)-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide (180 mg, 0.235 mmol, Intermediate 445) in EtOH (3 mL) was added hydrazine hydrate (620 mg, 10.5 mmol, 85% by weight) and the resulting mixture was stirred at 25° C. for 4 h. The mixture was diluted with H₂O (20 mL) and extracted with EtOAc (20 mL×3). The combined organic extracts were dried over anhydrous Na₂SO₄, filtered and concentrated to dryness to provide the title compound as a yellow oil, which was used without further purification.

### Intermediate 447

### N—((S)-1-(2-((1R,2R)-1-(((R)-tert-Butylsulfinyl)amino)-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide

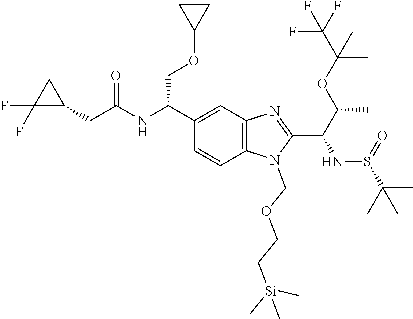 [SMILES: C[C@@H](OC(C)(C)C(F)(F)F)[C@H](N[S@](=O)C(C)(C)C)c1nc2cc([C@@H](COC3CC3)NC(=O)C[C@H]3CC3(F)F)ccc2n1COCC[Si](C)(C)C]

(S)-2-(2,2-Difluorocyclopropyl)acetic acid (35.1 mg, 0.258 mmol), HATU (97.9 mg, 0.258 mmol), DIPEA (0.150 mL, 0.858 mmol) and DCM (2 mL) were added to a vial, which was sealed and stirred for 10 min at rt. After this time, (R)—N-((1R,2R)-1-(5-((S)-1-amino-2-cyclopropoxyethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide (109 mg, 0.172 mmol, Intermediate 446) was added and the resulting mixture was stirred for 90 min at rt. The mixture was then diluted with H₂O (20 mL) and extracted with DCM (20 mL×3). The combined organic layers were dried over anhydrous Na₂SO₄, filtered, and concentrated to dryness to afford a yellow oil. This oil was subjected to silica gel chromatography (0-0.1% MeOH/DCM) to provide the title compound as a yellow solid (62% yield).

### Intermediate 448

### N—((S)-1-(2-((1R,2R)-1-Amino-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide

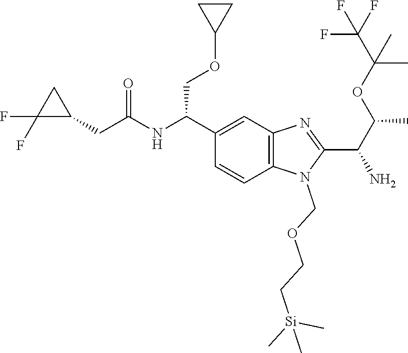 [SMILES: C[C@@H](OC(C)(C)C(F)(F)F)[C@H](N)c1nc2cc([C@@H](COC3CC3)NC(=O)C[C@H]3CC3(F)F)ccc2n1COCC[Si](C)(C)C]

To a solution of N—((S)-1-(2-((1R,2R)-1-(((R)-tert-butylsulfinyl)amino)-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide (110 mg, 0.146 mmol, Intermediate 447) in 1,4-dioxane (1 mL) was added HCl (1 mL, 4 M in 1,4-dioxane). The resulting mixture was stirred at 20° C. for 2 h. The mixture was diluted with H₂O (10 mL) and extracted with DCM (15 mL×2). The combined organic extracts were dried over anhydrous Na₂SO₄, filtered, and concentrated to dryness to afford the title compound as a yellow oil, which was used without further purification.

### Intermediate 449

### N—((S)-1-(2-((1R,2R)-1-Amino-2-((1,1,1-trifluoro-2-methylpropan-2-yl)oxy)propyl)-1H-benzo[d]imidazol-5-yl)-2-cyclopropoxyethyl)-2-((S)-2,2-difluorocyclopropyl)acetamide

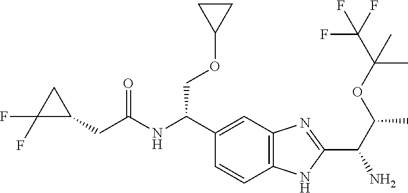 [SMILES: C[C@@H](OC(C)(C)C(F)(F)F)[C@H](N)c1nc2cc([C@@H](COC3CC3)NC(=O)C[C@H]3CC3(F)F)ccc2[nH]1]

A mixture of N—((S)-1-(2-((1R,2R)-1-amino-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide (110 mg, 0.170 mmol, Intermediate 448) in TFA (1 mL) was stirred at 20° C. for 1 h. The mixture was diluted with H₂O (10 mL) and NaHCO₃ (20 mL) and extracted with DCM (15 mL×2). The combined organic extracts were dried over anhydrous Na₂SO₄, filtered, and concentrated to dryness to provide the title compound as a yellow oil which was used without further purification.

### Intermediate 450

### N—((S)-1-(2-((1R,2R)-1-(((R)-tert-Butylsulfinyl)amino)-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide

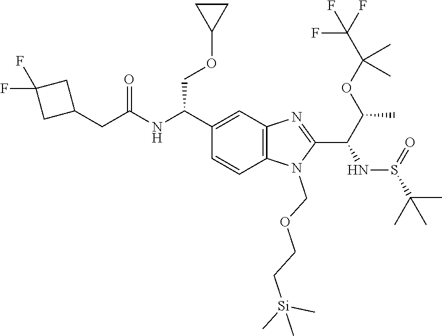 [SMILES: C[C@@H](OC(C)(C)C(F)(F)F)[C@H](N[S@](=O)C(C)(C)C)c1nc2cc([C@@H](COC3CC3)NC(=O)CC3CC(F)(F)C3)ccc2n1COCC[Si](C)(C)C]

The title compound was prepared as described for the synthesis of Intermediate 447 using 2-(3,3-difluorocyclobutyl)acetic acid in place of (S)-2-(2,2-difluorocyclopropyl)acetic acid. The material was purified by silica gel chromatography (0-0.5% MeOH/DCM) to provide the title compound as a yellow oil (81% yield).

### Intermediate 451

### N—((S)-1-(2-((1R,2R)-1-Amino-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide

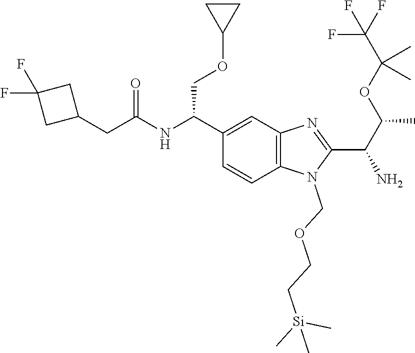 [SMILES: C[C@@H](OC(C)(C)C(F)(F)F)[C@H](N)c1nc2cc([C@@H](COC3CC3)NC(=O)CC3CC(F)(F)C3)ccc2n1COCC[Si](C)(C)C]

The title compound was prepared as described for the synthesis of Intermediate 448 using N—((S)-1-(2-((1R,2R)-1-(((R)-tert-butylsulfinyl)amino)-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide (Intermediate 450) in place of N—((S)-1-(2-((1R,2R)-1-(((R)-tert-butylsulfinyl)amino)-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide and was used without further purification.

### Intermediate 452

### N—((S)-1-(2-((1R,2R)-1-Amino-2-((1,1,1-trifluoro-2-methylpropan-2-yl)oxy)propyl)-1H-benzo[d]imidazol-5-yl)-2-cyclopropoxyethyl)-2-(3,3-difluorocyclobutyl)acetamide

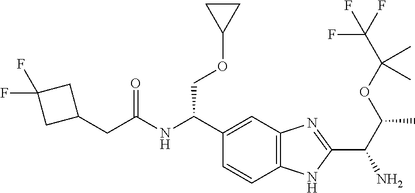 [SMILES: C[C@@H](OC(C)(C)C(F)(F)F)[C@H](N)c1nc2cc([C@@H](COC3CC3)NC(=O)CC3CC(F)(F)C3)ccc2[nH]1]

The title compound was prepared as described for the synthesis of Intermediate 449 using N—((S)-1-(2-((1R,2R)-1-amino-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide (Intermediate 451) in place of N—((S)-1-(2-((1R,2R)-1-amino-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide and was used without further purification.

### Intermediate 453

### (2R)-2-((1,1,1-Trifluoropropan-2-yl)oxy)propanoic acid

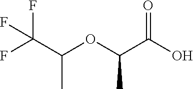

The title compound was prepared as described for the synthesis of Intermediate 441 using 1,1,1-trifluoropropan-2-ol in place of 1,1,1-trifluoro-2-methylpropan-2-ol and was used without further purification.

### Intermediate 454

### (R)—N-Methoxy-N-methyl-2-(((S)-1,1,1-trifluoropropan-2-yl)oxy)propanamide

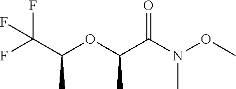

The title compound (30% yield) was prepared as described for the synthesis of Intermediate 442 using (2R)-2-((1,1,1-trifluoropropan-2-yl)oxy)propanoic acid (Intermediate 453) in place of (R)-2-((1,1,1-trifluoro-2-methylpropan-2-yl)oxy)propanoic acid. The material was subjected to silica gel chromatography (0-50% EtOAc/petroleum ether) to give three isomers, with the title compound eluting as the third isomer.

### Intermediate 455

### (R)-2-(((S)-1,1,1-Trifluoropropan-2-yl)oxy)propanal

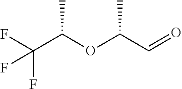

The title compound was prepared as described for the synthesis of Intermediate 443 using (R)—N-methoxy-N-methyl-2-(((S)-1,1,1-trifluoropropan-2-yl)oxy)propanamide (Intermediate 454) in place of (R)—N-methoxy-N-methyl-2-((1,1,1-trifluoro-2-methylpropan-2-yl)oxy)propanamide and was used without further purification.

### Intermediate 456

### (R)-2-Methyl-N—((R,E)-2-(((S)-1,1,1-trifluoropropan-2-yl)oxy)propylidene)propane-2-sulfinamide

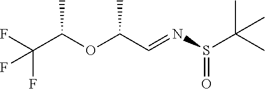 [SMILES: C[C@H](/C=N/[S@](=O)C(C)(C)C)O[C@@H](C)C(F)(F)F]

The title compound was prepared as described for the synthesis of Intermediate 444 using (R)-2-(((S)-1,1,1-trifluoropropan-2-yl)oxy)propanal (Intermediate 455) in place of (R)-2-((1,1,1-trifluoro-2-methylpropan-2-yl)oxy)propanal. The material was subjected to silica gel chromatography (0-15% EtOAc/petroleum ether) to provide the title compound as a light-yellow oil (25% yield).

### Intermediate 457

### (R)—N-((1R,2R)-1-(5-((S)-2-Cyclopropoxy-1-(1,3-dioxoisoindolin-2-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

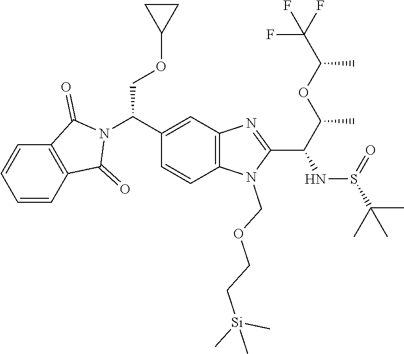 [SMILES: C[C@H](O[C@H](C)[C@H](N[S@](=O)C(C)(C)C)c1nc2cc([C@@H](COC3CC3)N3C(=O)c4ccccc4C3=O)ccc2n1COCC[Si](C)(C)C)C(F)(F)F]

The title compound was prepared as described for the synthesis of Intermediate 445 using (R)-2-methyl-N—((R,E)-2-(((S)-1,1,1-trifluoropropan-2-yl)oxy)propylidene)propane-2-sulfinamide (Intermediate 456) in place of (R)-2-methyl-N—((R,E)-2-((1,1,1-trifluoro-2-methylpropan-2-yl)oxy)propylidene)propane-2-sulfinamide. The material was initially purified by silica gel chromatography (0-100% EtOAc/petroleum ether) and then subsequently purified by silica gel chromatography (0-0.5% MeOH/DCM) to provide the title compound as a black oil (42% yield).

### Intermediate 458

### (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide

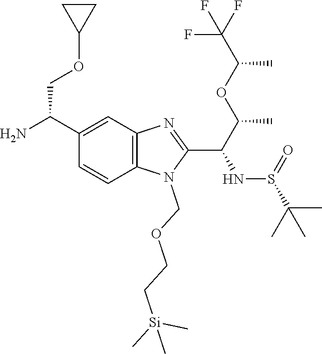 [SMILES: C[C@H](O[C@H](C)[C@H](N[S@](=O)C(C)(C)C)c1nc2cc([C@H](N)COC3CC3)ccc2n1COCC[Si](C)(C)C)C(F)(F)F]

The title compound was prepared as described for the synthesis of Intermediate 446 using (R)—N-((1R,2R)-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (Intermediate 457) in place of (R)—N-((1R,2R)-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide and was used without further purification.

### Intermediate 459

### N—((S)-1-(2-((1R,2R)-1-(((R)-tert-Butylsulfinyl)amino)-2-(((S)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide

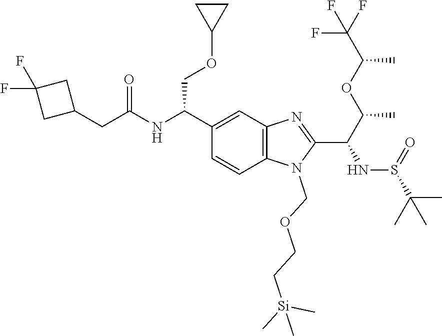 [SMILES: C[C@H](O[C@H](C)[C@H](N[S@](=O)C(C)(C)C)c1nc2cc([C@@H](COC3CC3)NC(=O)CC3CC(F)(F)C3)ccc2n1COCC[Si](C)(C)C)C(F)(F)F]

A mixture of 2-(3,3-difluorocyclobutyl)acetic acid (160 mg, 1.06 mmol), DIPEA (0.28 mL, 1.60 mmol), EDCI (204 mg, 1.06 mmol) and HOBt (86.2 mg, 0.638 mmol) in DCM (4 mL) was stirred at rt for 30 min. Then, (R)—N-((1R,2R)-1-(5-((S)-1-amino-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide (330 mg, 0.532 mmol, Intermediate 458) was added and the resulting mixture was stirred at rt for 16 h. Water (20 mL) was added and the mixture was extracted with DCM (20 mL×2). The organic layers were combined, washed with brine (50 mL), dried over anhydrous Na₂SO₄, filtered, and concentrated to dryness to give a yellow oil. The oil was then subjected to silica gel chromatography (0-0.7% MeOH/DCM) to provide the title compound as a yellow oil (80% yield).

### Intermediate 460

### N—((S)-1-(2-((1R,2R)-1-Amino-2-(((S)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide

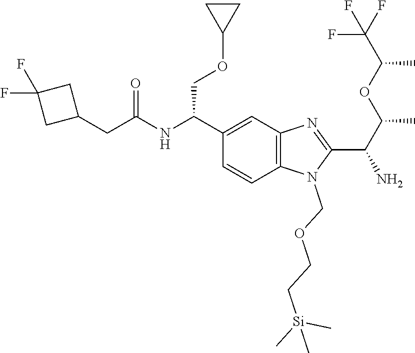 [SMILES: C[C@H](O[C@H](C)[C@H](N)c1nc2cc([C@@H](COC3CC3)NC(=O)CC3CC(F)(F)C3)ccc2n1COCC[Si](C)(C)C)C(F)(F)F]

The title compound was prepared as described for the synthesis of Intermediate 448 using N—((S)-1-(2-((1R,2R)-1-(((R)-tert-butylsulfinyl)amino)-2-(((S)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide (Intermediate 459) in place of N—((S)-1-(2-((1R,2R)-1-(((R)-tert-butylsulfinyl)amino)-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide and was used without further purification.

### Intermediate 461

### N—((S)-1-(2-((1R,2R)-1-Amino-2-(((S)-1,1,1-trifluoropropan-2-yl)oxy)propyl)-1H-benzo[d]imidazol-5-yl)-2-cyclopropoxyethyl)-2-(3,3-difluorocyclobutyl)acetamide

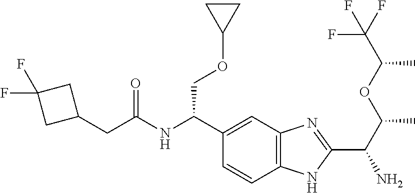 [SMILES: C[C@H](O[C@H](C)[C@H](N)c1nc2cc([C@@H](COC3CC3)NC(=O)CC3CC(F)(F)C3)ccc2[nH]1)C(F)(F)F]

The title compound (96% yield) was prepared as described for the synthesis of Intermediate 449 using N—((S)-1-(2-((1R,2R)-1-amino-2-(((S)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide (Intermediate 460) in place of N—((S)-1-(2-((1R,2R)-1-amino-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide.

### Intermediate 462

### (R)—N-Methoxy-N-methyl-2-(((R)-1,1,1-trifluoropropan-2-yl)oxy)propanamide

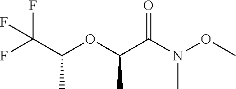

The title compound (30% yield) was prepared as described for the synthesis of Intermediate 442 using (2R)-2-((1,1,1-trifluoropropan-2-yl)oxy)propanoic acid (Intermediate 453) in place of (R)-2-((1,1,1-trifluoro-2-methylpropan-2-yl)oxy)propanoic acid. The material was then subjected to silica gel chromatography (0-12% EtOAc/petroleum ether) to give three products, with the title compound eluting as the first isomer.

### Intermediate 463

### (R)-2-(((R)-1,1,1-Trifluoropropan-2-yl)oxy)propanal

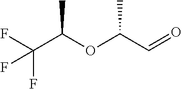

The title compound was prepared as described for the synthesis of Intermediate 443 using (R)—N-methoxy-N-methyl-2-(((R)-1,1,1-trifluoropropan-2-yl)oxy)propanamide (Intermediate 462) in place of (R)—N-methoxy-N-methyl-2-((1,1,1-trifluoro-2-methylpropan-2-yl)oxy)propanamide and was used without further purification.

### Intermediate 464

### (R)-2-Methyl-N—((R,E)-2-(((R)-1,1,1-trifluoropropan-2-yl)oxy)propylidene)propane-2-sulfinamide

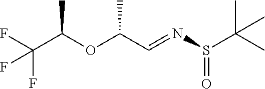 [SMILES: C[C@H](/C=N/[S@](=O)C(C)(C)C)O[C@H](C)C(F)(F)F]

The title compound was prepared as described for the synthesis of Intermediate 444 using (R)-2-(((R)-1,1,1-trifluoropropan-2-yl)oxy)propanal (Intermediate 463) in place of (R)-2-((1,1,1-trifluoro-2-methylpropan-2-yl)oxy)propanal. The material was subjected to silica gel chromatography (0-10% EtOAc/petroleum ether) to provide the title compound as a light-yellow oil (18% yield).

### Intermediate 465

### (R)—N-((1R,2R)-1-(5-((S)-2-Cyclopropoxy-1-(1,3-dioxoisoindolin-2-yl)ethyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

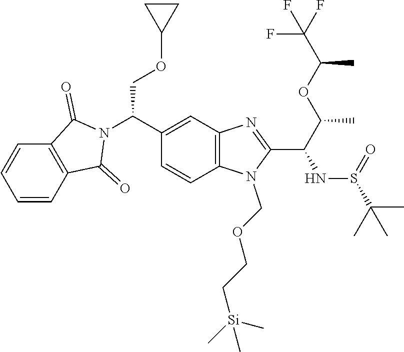

The title compound was prepared as described for the synthesis of Intermediate 445 using (R)-2-methyl-N—((R,E)-2-(((R)-1,1,1-trifluoropropan-2-yl)oxy)propylidene)propane-2-sulfinamide (Intermediate 464) in place of (R)-2-methyl-N—((R,E)-2-((1,1,1-trifluoro-2-methylpropan-2-yl)oxy)propylidene)propane-2-sulfinamide. The material was subjected to silica gel chromatography (0-0.2% MeOH/DCM) to provide the title compound as a yellow oil (39% yield).

### Intermediate 466

### (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide

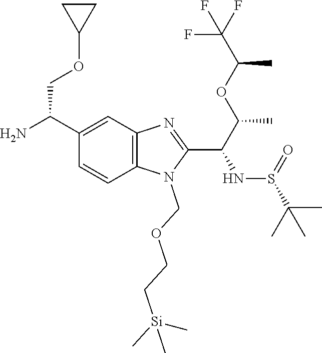

The title compound was prepared as described for the synthesis of Intermediate 446 using (R)—N-((1R,2R)-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (Intermediate 465) in place of (R)—N-((1R,2R)-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide and was used without further purification.

### Intermediate 467

### N—((S)-1-(2-((1R,2R)-1-(((R)-tert-Butylsulfinyl)amino)-2-(((R)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide

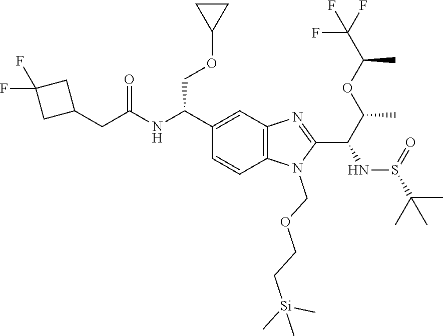

A mixture of 2-(3,3-difluorocyclobutyl)acetic acid (56.6 mg, 0.377 mmol), DCM (5 mL), DIPEA (0.14 mL, 0.75 mmol) and HATU (191.1 mg, 0.503 mmol) was stirred at rt for 15 min, and then (R)—N-((1R,2R)-1-(5-((S)-1-amino-2-cyclopropoxyethyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (156 mg, 0.25 mmol, Intermediate 466) was added. The resulting mixture was stirred at rt for 90 min before HCl (3 mL, 2 M aqueous) was added dropwise. The mixture was extracted with EtOAc (50 mL×3), the organic layers combined, washed sequentially with saturated aqueous NaHCO₃ (3×50 mL) and brine (10 mL×2), dried over anhydrous Na₂SO₄, filtered, and concentrated to dryness to afford a colorless oil. This oil was subjected to silica gel chromatography (0-5% MeOH/DCM) to provide the title compound as a colorless oil (75% yield).

### Intermediate 468

### N—((S)-1-(2-((1R,2R)-1-Amino-2-(((R)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide

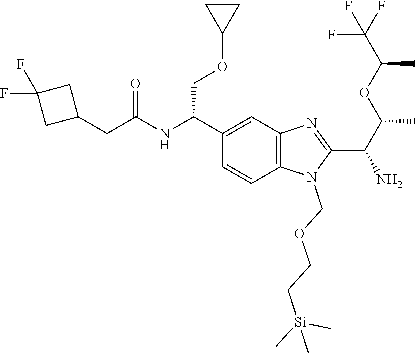 [SMILES: C[C@@H](O[C@H](C)C(F)(F)F)[C@H](N)c1nc2cc([C@@H](COC3CC3)NC(=O)CC3CC(F)(F)C3)ccc2n1COCC[Si](C)(C)C]

The title compound was prepared as described for the synthesis of Intermediate 448 using N—((S)-1-(2-((1R,2R)-1-(((R)-tert-butylsulfinyl)amino)-2-(((R)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide (Intermediate 467) in place of N—((S)-1-(2-((1R,2R)-1-(((R)-tert-butylsulfinyl)amino)-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide and was used without further purification.

### Intermediate 469

### N—((S)-1-(2-((1R,2R)-1-Amino-2-(((R)-1,1,1-trifluoropropan-2-yl)oxy)propyl)-1H-benzo[d]imidazol-5-yl)-2-cyclopropoxyethyl)-2-(3,3-difluorocyclobutyl)acetamide trifluoroacetate

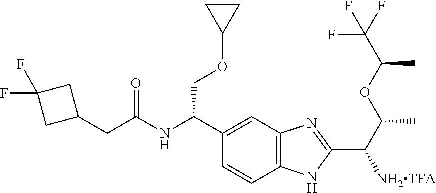

The title compound was prepared as described in the synthesis of Intermediate 449 using N—((S)-1-(2-((1R,2R)-1-amino-2-(((R)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-(3,3-difluorocyclobutyl)acetamide (Intermediate 468) in place of N—((S)-1-(2-((1R,2R)-1-amino-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide. The reaction mixture was concentrated to dryness to provide the title compound, which was used without further purification.

### Intermediate 470

### N—((S)-1-(2-((1R,2R)-1-(((R)-tert-Butylsulfinyl)amino)-2-(((R)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide

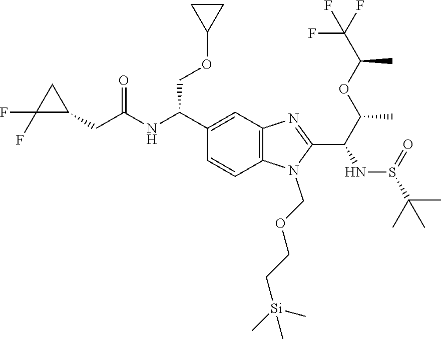

The title compound was prepared as described for the synthesis of Intermediate 447 using (R)—N-((1R,2R)-1-(5-((S)-1-amino-2-cyclopropoxyethyl)-1-((2-(trimethylsilyl)ethoxy)methyl)-1H-benzo[d]imidazol-2-yl)-2-(((R)-1,1,1-trifluoropropan-2-yl)oxy)propyl)-2-methylpropane-2-sulfinamide (Intermediate 466) in place of (R)—N-((1R,2R)-1-(5-((S)-1-amino-2-cyclopropoxyethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide. The material was purified via silica gel chromatography (0-1.1% MeOH/DCM) to provide the title compound as a yellow oil (54% yield).

### Intermediate 471

### N—((S)-1-(2-((1R,2R)-1-Amino-2-(((R)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide

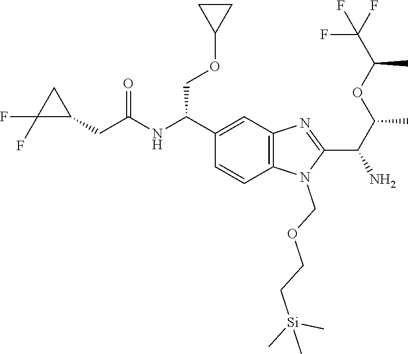 [SMILES: C[C@@H](O[C@H](C)C(F)(F)F)[C@H](N)c1nc2cc([C@@H](COC3CC3)NC(=O)C[C@H]3CC3(F)F)ccc2n1COCC[Si](C)(C)C]

The title compound was prepared as described for the synthesis of Intermediate 448 using N—((S)-1-(2-((1R,2R)-1-(((R)-tert-butylsulfinyl)amino)-2-(((R)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide (Intermediate 470) in place of N—((S)-1-(2-((1R,2R)-1-(((R)-tert-butylsulfinyl)amino)-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide and was used without further purification.

### Intermediate 472

### N—((S)-1-(2-((1R,2R)-1-Amino-2-(((R)-1,1,1-trifluoropropan-2-yl)oxy)propyl)-1H-benzo[d]imidazol-5-yl)-2-cyclopropoxyethyl)-2-((S)-2,2-difluorocyclopropyl)acetamide trifluoroacetate

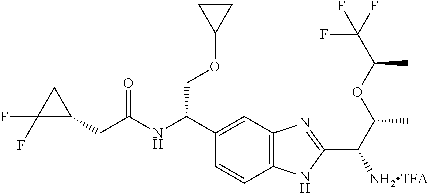

The title compound was prepared as described for the synthesis of Intermediate 449 using N—((S)-1-(2-((1R,2R)-1-amino-2-(((R)-1,1,1-trifluoropropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide (Intermediate 471) in place of N—((S)-1-(2-((1R,2R)-1-amino-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide. The reaction mixture was concentrated to dryness to provide the title compound, which was used without further purification.

### Intermediate 473

### (R)-2-Cyclopropoxypropanoic acid

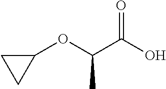

The title compound was prepared as described for the synthesis of Intermediate 441 using cyclopropanol in place of 1,1,1-trifluoro-2-methylpropan-2-ol and was used without further purification.

### Intermediate 474

### (R)-2-Cyclopropoxy-N-methoxy-N-methylpropanamide

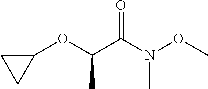

The title compound was prepared as described for the synthesis of Intermediate 442 using (R)-2-cyclopropoxypropanoic acid (Intermediate 473) in place of (R)-2-((1,1,1-trifluoro-2-methylpropan-2-yl)oxy)propanoic acid. This material was purified by silica gel chromatography (0-30% EtOAc/petroleum ether) to provide the title compound as a yellow oil (88% yield).

### Intermediate 475

### (R)-2-Cyclopropoxypropanal

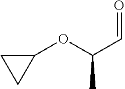

The title compound was prepared as described for the synthesis of Intermediate 443 using (R)-2-cyclopropoxy-N-methoxy-N-methylpropanamide (Intermediate 474) in place of (R)—N-methoxy-N-methyl-2-((1,1,1-trifluoro-2-methylpropan-2-yl)oxy)propanamide and was used without further purification.

### Intermediate 476

### (R)—N—((R,E)-2-Cyclopropoxypropylidene)-2-methylpropane-2-sulfinamide

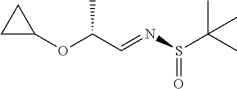

The title compound was prepared as described for the synthesis of Intermediate 444 using (R)-2-cyclopropoxypropanal (Intermediate 475) in place of (R)-2-((1,1,1-trifluoro-2-methylpropan-2-yl)oxy)propanal. This material was purified by silica gel chromatography (0-20% EtOAc/petroleum ether) to provide the title compound as a light-yellow oil (13% yield).

### Intermediate 477

### (R)—N-((1R,2R)-2-Cyclopropoxy-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)propyl)-2-methylpropane-2-sulfinamide

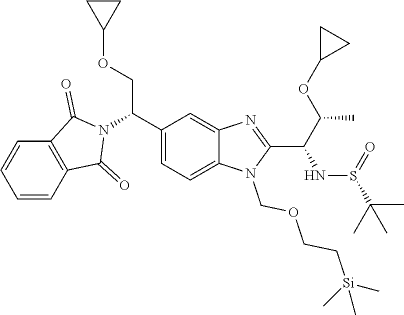

The title compound was prepared as described for the synthesis of Intermediate 445 using (R)—N—((R,E)-2-cyclopropoxypropylidene)-2-methylpropane-2-sulfinamide (Intermediate 476) in place of (R)-2-methyl-N—((R,E)-2-((1,1,1-trifluoro-2-methylpropan-2-yl)oxy)propylidene)propane-2-sulfinamide. The material was purified by silica gel chromatography (0-5% MeOH/DCM) to afford the title compound as a colorless oil (45% yield).

### Intermediate 478

### (R)—N-((1R,2R)-1-(5-((S)-1-Amino-2-cyclopropoxyethyl)-1-((2-(trimethylsilyl)ethoxy)methyl)-1H-benzo[d]imidazol-2-yl)-2-cyclopropoxypropyl)-2-methylpropane-2-sulfinamide

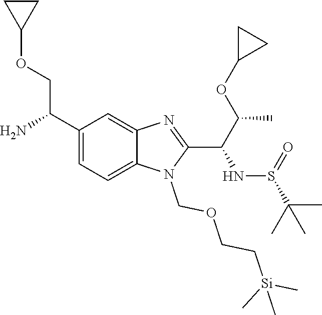 [SMILES: C[C@@H](OC1CC1)[C@H](N[S@](=O)C(C)(C)C)c1nc2cc([C@H](N)COC3CC3)ccc2n1COCC[Si](C)(C)C]

The title compound (59% yield) was prepared as described for the synthesis of Intermediate 446 using (R)—N-((1R,2R)-2-cyclopropoxy-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)propyl)-2-methylpropane-2-sulfinamide (Intermediate 477) in place of (R)—N-((1R,2R)-1-(5-((S)-2-cyclopropoxy-1-(1,3-dioxoisoindolin-2-yl)ethyl)-1-((2-(trimethylsilyl)ethoxy)methyl)-1H-benzo[d]imidazol-2-yl)-2-((1,1,1-trifluoro-2-methylpropan-2-yl)oxy)propyl)-2-methylpropane-2-sulfinamide.

### Intermediate 479

### N—((S)-1-(2-((1R,2R)-1-(((R)-tert-Butylsulfinyl)amino)-2-cyclopropoxypropyl)-1-((2-(trimethylsilyl)ethoxy)methyl)-1H-benzo[d]imidazol-5-yl)-2-cyclopropoxyethyl)-2-(3,3-difluorocyclobutyl)acetamide

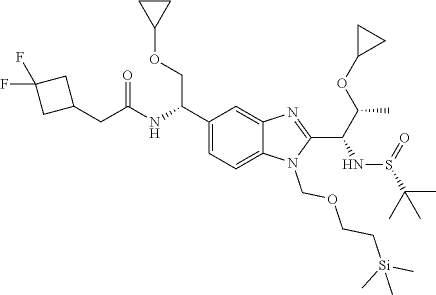 [SMILES: C[C@@H](OC1CC1)[C@H](N[S@](=O)C(C)(C)C)c1nc2cc([C@@H](COC3CC3)NC(=O)CC3CC(F)(F)C3)ccc2n1COCC[Si](C)(C)C]

The title compound was prepared as described for the synthesis of Intermediate 459 using (R)—N-((1R,2R)-1-(5-((S)-1-amino-2-cyclopropoxyethyl)-1-((2-(trimethylsilyl)ethoxy)methyl)-1H-benzo[d]imidazol-2-yl)-2-cyclopropoxypropyl)-2-methylpropane-2-sulfinamide (Intermediate 478) in place of (R)—N-((1R,2R)-1-(5-((S)-1-amino-2-cyclopropoxyethyl)-1-((2-(trimethylsilyl)ethoxy)methyl)-1H-benzo[d]imidazol-2-yl)-2-(((S)-1,1,1-trifluoropropan-2-yl)oxy)propyl)-2-methylpropane-2-sulfinamide. The material was subjected to silica gel chromatography (0-0.7% MeOH/DCM) to provide the title compound as a colorless oil (53% yield).

### Intermediate 480

### N—((S)-1-(2-((1R,2R)-1-Amino-2-cyclopropoxypropyl)-1-((2-(trimethylsilyl)ethoxy)methyl)-1H-benzo[d]imidazol-5-yl)-2-cyclopropoxyethyl)-2-(3,3-difluorocyclobutyl)acetamide

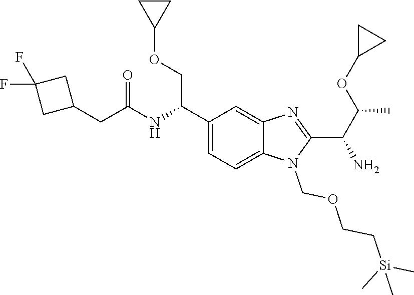 [SMILES: C[C@@H](OC1CC1)[C@H](N)c1nc2cc([C@@H](COC3CC3)NC(=O)CC3CC(F)(F)C3)ccc2n1COCC[Si](C)(C)C]

The title compound (95% yield) was prepared as described for the synthesis of Intermediate 448 using N—((S)-1-(2-((1R,2R)-1-(((R)-tert-butylsulfinyl)amino)-2-cyclopropoxypropyl)-1-((2-(trimethylsilyl)ethoxy)methyl)-1H-benzo[d]imidazol-5-yl)-2-cyclopropoxyethyl)-2-(3,3-difluorocyclobutyl)acetamide (Intermediate 479) in place of N—((S)-1-(2-((1R,2R)-1-(((R)-tert-butylsulfinyl)amino)-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide.

### Intermediate 481

### N—((S)-1-(2-((1R,2R)-1-Amino-2-cyclopropoxypropyl)-1H-benzo[d]imidazol-5-yl)-2-cyclopropoxyethyl)-2-(3,3-difluorocyclobutyl)acetamide trifluoroacetate

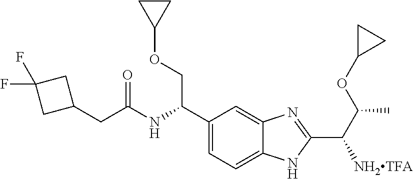

The title compound was prepared as described for the synthesis of Intermediate 449 using N—((S)-1-(2-((1R,2R)-1-amino-2-cyclopropoxypropyl)-1-((2-(trimethylsilyl)ethoxy)methyl)-1H-benzo[d]imidazol-5-yl)-2-cyclopropoxyethyl)-2-(3,3-difluorocyclobutyl)acetamide (Intermediate 480) in place of N—((S)-1-(2-((1R,2R)-1-amino-2-((1,1,1-trifluoro-2-methylpropan-2-yl)oxy)propyl)-1-((2-(trimethylsilyl)ethoxy)methyl)-1H-benzo[d]imidazol-5-yl)-2-cyclopropoxyethyl)-2-((S)-2,2-difluorocyclopropyl)acetamide. The reaction mixture was concentrated to dryness to provide the title compound, which was used without further purification.

### Intermediate 482

### 2,5-Dioxopyrrolidin-1-yl 4-cyclopropyl-1,2,5-oxadiazole-3-carboxylate

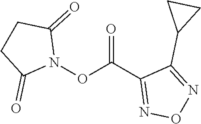 [SMILES: O=C(ON1C(=O)CCC1=O)c1nonc1C1CC1]

Step A: 4-Cyclopropyl-1,2,5-oxadiazole-3-carbonyl chloride. A flask was charged with 4-cyclopropyl-1,2,5-oxadiazole-3-carboxylic acid (200 mg, 1.3 mmol) and DCM (2.5 mL), and the mixture was cooled to 0° C. Then, oxalyl chloride (0.22 mL, 2.6 mmol) and one drop of DMF were added. The resulting mixture was stirred for 1 h as it warmed to rt. The mixture was concentrated to a yellow oil and was used without further purification.

Step B: 2,5-Dioxopyrrolidin-1-yl 4-cyclopropyl-1,2,5-oxadiazole-3-carboxylate. The residue from Step A was dissolved in DCM (3.2 mL) and cooled to 0° C. To the solution were added N-hydroxysuccinimide (231 mg, 1.9 mmol) and DIPEA (0.34 mL, 1.9 mmol). The resulting mixture was stirred for 1 h as it warmed to rt. The reaction was quenched by the addition of water (3 mL) and the layers were separated. The organic layer was washed with brine, dried over anhydrous MgSO₄, filtered and concentrated to dryness. This material was purified by silica gel chromatography (0-100% ethyl acetate (with 10% MeOH)/hexanes) to afford the title compound as a clear oil (40% yield).

### Example 1

### N—[(S)-(4,4-Difluorocyclohexyl)-[6-[(1R)-1-(4,4,4-trifluorobutanoylamino)ethyl]-1H-benzimidazol-2-yl]methyl]-1-(3,3,3-trifluoropropyl)pyrazole-4-carboxamide

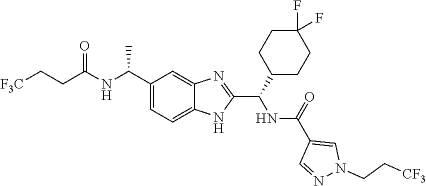 [SMILES: C[C@@H](NC(=O)CCC(F)(F)F)c1ccc2[nH]c([C@@H](NC(=O)c3cnn(CCC(F)(F)F)c3)C3CCC(F)(F)CC3)nc2c1]

A mixture of N—((R)-1-(2-((S)-amino(4,4-difluorocyclohexyl)methyl)-1H-benzo[d]imidazol-5-yl)ethyl)-4,4,4-trifluorobutanamide (75 mg, 0.17 mmol, Intermediate 4), 1-(3,3,3-trifluoropropyl)-1H-pyrazole-4-carboxylic acid (41.5 mg, 0.2 mmol), HOBt (24.6 mg, 0.18 mmol), DIPEA (36 μL, 0.21 mmol) and ACN (1.9 mL) was stirred until homogeneous. Then, EDCI (35 mg, 0.18 mmol) was added and the resulting mixture stirred at rt for 3 h. After this time, water was added and the mixture was extracted with EtOAc (2×20 mL). The organic layers were combined, washed with brine (20 mL), dried over anhydrous Na₂SO₄, filtered, and concentrated to dryness. The residue was purified by silica gel chromatography (0-100% (10% 2 M NH₃ in MeOH/DCM)/DCM) to provide the title compound as a white solid. ¹H NMR (400 MHz, CDCl₃) δ 12.04 (s, 1H), 8.18-7.95 (m, 2H), 7.58-7.49 (m, 1H), 7.37-7.29 (m, 1H), 7.18 (t, J=9.8 Hz, 1H), 6.77-6.63 (m, 1H), 5.21-5.04 (m, 2H), 4.37-4.29 (m, 2H), 2.78-2.68 (m, 2H), 2.65-2.35 (m, 10H), 2.27-2.11 (m, 2H), 2.10-1.96 (m, 2H), 1.84-1.55 (m, 2H), 1.41-1.28 (m, 1H). MS (ESI) m/z: [M+H]⁺ Found 623.3.

### Example 2

### N—[(S)-(4,4-Difluorocyclohexyl)-[6-[(1R)-1-(4,4,4-trifluorobutanoylamino)ethyl]-1H-benzimidazol-2-yl]methyl]-1-(3,3,3-trifluoropropyl)pyrazole-3-carboxamide

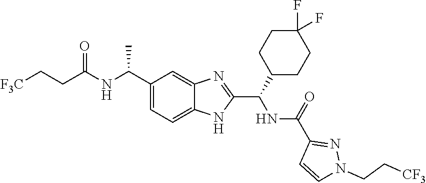 [SMILES: C[C@@H](NC(=O)CCC(F)(F)F)c1ccc2[nH]c([C@@H](NC(=O)c3ccn(CCC(F)(F)F)n3)C3CCC(F)(F)CC3)nc2c1]

The title compound was prepared as described for the synthesis of Example 1, using 1-(3,3,3-trifluoropropyl)-1H-pyrazole-3-carboxylic acid in place of 1-(3,3,3-trifluoropropyl)-1H-pyrazole-4-carboxylic acid, and stirring at 40° C. for 45 min after stirring at rt for 3 h. ¹H NMR (400 MHz, CDCl₃) δ 11.77 (s, 1H), 7.95-7.59 (m, 1H), 7.43 (d, J=2.4 Hz, 1H), 7.39-7.32 (m, 1H), 7.19 (t, J=9.3 Hz, 1H), 6.79-6.73 (m, 1H), 6.61 (dd, J=33.6, 7.7 Hz, 1H), 5.23-5.07 (m, 2H), 4.36 (t, J=7.2 Hz, 2H), 2.82-2.67 (m, 2H), 2.54-2.29 (m, 6H), 2.23 (s, 4H), 2.18-1.97 (m, 3H), 1.85-1.56 (m, 3H), 1.44-1.31 (m, 1H). MS (ESI) m/z: [M+H]⁺ Found 623.3.

### Example 3

### N—[(S)-(4,4-Difluorocyclohexyl)-[6-[(1R)-1-(4,4,4-trifluorobutanoylamino)ethyl]-1H-benzimidazol-2-yl]methyl]-2-(3,3,3-trifluoropropyl)triazole-4-carboxamide

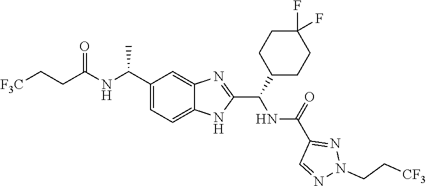 [SMILES: C[C@@H](NC(=O)CCC(F)(F)F)c1ccc2[nH]c([C@@H](NC(=O)c3cnn(CCC(F)(F)F)n3)C3CCC(F)(F)CC3)nc2c1]

The title compound was prepared as described for the synthesis of Example 1, using 2-(3,3,3-trifluoropropyl)-2H-1,2,3-triazole-4-carboxylic acid (Intermediate 7) in place of 1-(3,3,3-trifluoropropyl)-1H-pyrazole-4-carboxylic acid, and stirring at rt for 2 h followed by 40° C. for 2.5 h instead of stirring at rt for 3 h. ¹H NMR (400 MHz, CDCl₃) δ 11.37 (d, J=58.4 Hz, 1H), 7.98 (d, J=4.5 Hz, 1H), 7.95-7.88 (m, 1H), 7.63-7.56 (m, 1H), 7.31-7.13 (m, 2H), 6.05 (dd, J=50.2, 7.6 Hz, 1H), 5.22-5.10 (m, 2H), 4.65-4.56 (m, 2H), 2.87-2.74 (m, 2H), 2.51-2.31 (m, 5H), 2.18-1.98 (m, 3H), 1.78-1.56 (m, 3H), 1.54-1.36 (m, 5H). MS (ESI) m/z: [M+H]⁺ Found 624.3.

### Example 4

### N—[(S)-(4,4-Difluorocyclohexyl)-[6-[(1R)-1-(4,4,4-trifluorobutanoylamino)ethyl]-1H-benzimidazol-2-yl]methyl]-3-(3,3,3-trifluoropropyl)triazole-4-carboxamide

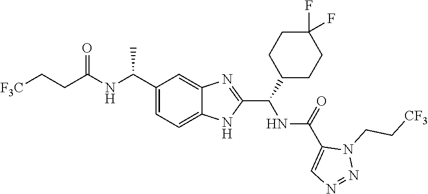 [SMILES: C[C@@H](NC(=O)CCC(F)(F)F)c1ccc2[nH]c([C@@H](NC(=O)c3cnnn3CCC(F)(F)F)C3CCC(F)(F)CC3)nc2c1]

The title compound was prepared as described for the synthesis of Example 1, using 1-(3,3,3-trifluoropropyl)-1H-1,2,3-triazole-5-carboxylic acid (Intermediate 8) in place of 1-(3,3,3-trifluoropropyl)-1H-pyrazole-4-carboxylic acid, and stirring at rt for 19.5 h instead of 3 h. The title compound was also further purified by acidic preparative HPLC (TFA) and then free-based by concentrating to dryness, dissolving the residue in water, adjusting the pH to ˜8 by the addition of 1 N aqueous NaOH and extracting with DCM (2×20 mL). The organic layers were combined, dried over anhydrous Na₂SO₄, filtered and concentrated to dryness to provide the title compound as a white solid. ¹H NMR (400 MHz, CDCl₃) δ 11.96-11.39 (m, 1H), 9.01-8.69 (m, 1H), 8.23 (s, 1H), 7.64-7.30 (m, 2H), 7.19 (d, J=8.4 Hz, 1H), 6.49 (d, J=7.7 Hz, 1H), 5.22-5.10 (m, 1H), 5.08-4.99 (m, 1H), 4.94 (t, J=7.5 Hz, 2H), 2.83-2.69 (m, 2H), 2.47-2.36 (m, 4H), 2.13-2.08 (m, 1H), 2.04-1.94 (m, 2H), 1.79-1.58 (m, 2H), 1.52-1.28 (m, 6H), 1.27-1.16 (m, 1H). MS (ESI) m/z: [M+H]⁺ Found 624.2.

### Example 5

### N—[(S)-(4,4-Difluorocyclohexyl)-[6-[(1R)-1-(4,4,4-trifluorobutanoylamino)ethyl]-1H-benzimidazol-2-yl]methyl]-5-methyl-1-(3,3,3-trifluoropropyl)pyrazole-4-carboxamide

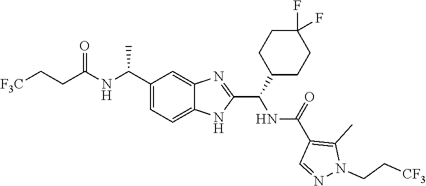 [SMILES: Cc1c(C(=O)N[C@H](c2nc3cc([C@@H](C)NC(=O)CCC(F)(F)F)ccc3[nH]2)C2CCC(F)(F)CC2)cnn1CCC(F)(F)F]

The title compound was prepared as described for the synthesis of Example 1, using 5-methyl-1-(3,3,3-trifluoropropyl)-1H-pyrazole-4-carboxylic acid in place of 1-(3,3,3-trifluoropropyl)-1H-pyrazole-4-carboxylic acid, and stirring at rt for 5 h instead of 3 h. ¹H NMR (400 MHz, CDCl₃) δ 11.91 (s, 1H), 7.97-7.88 (m, 1H), 7.61-7.54 (m, 1H), 7.40-7.32 (m, 1H), 7.23-7.14 (m, 1H), 6.73-6.58 (m, 1H), 5.22-5.10 (m, 1H), 5.10-5.00 (m, 1H), 4.27 (t, J=7.1 Hz, 2H), 2.75-2.62 (m, 3H), 2.47-2.35 (m, 4H), 2.25-1.98 (m, 4H), 1.85-1.62 (m, 2H), 1.61-1.26 (m, 7H). MS (ESI) m/z: [M+H]⁺ Found 637.2.

### Example 6

### N—[(S)-(4,4-Difluorocyclohexyl)-[6-[(1R)-1-(4,4,4-trifluorobutanoylamino)ethyl]-1H-benzimidazol-2-yl]methyl]-1-methyl-5-(3,3,3-trifluoropropyl)pyrazole-4-carboxamide

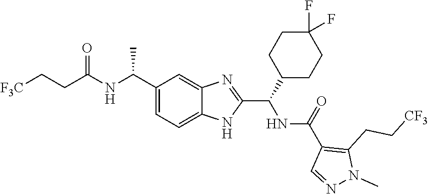 [SMILES: C[C@@H](NC(=O)CCC(F)(F)F)c1ccc2[nH]c([C@@H](NC(=O)c3cnn(C)c3CCC(F)(F)F)C3CCC(F)(F)CC3)nc2c1]

The title compound was prepared as described for the synthesis of Example 1, using 1-methyl-5-(3,3,3-trifluoropropyl)-1H-pyrazole-4-carboxylic acid in place of 1-(3,3,3-trifluoropropyl)-1H-pyrazole-4-carboxylic acid, and stirring at rt for 2 h followed by 40° C. for 1.5 h instead of stirring at rt for 3 h. The title compound was also further purified by basic preparative HPLC (NH₄OH). ¹H NMR (400 MHz, CDCl₃) δ 7.92-7.87 (m, 1H), 7.65-7.56 (m, 1H), 7.43-7.37 (m, 1H), 7.24-7.17 (m, 1H), 5.24-5.13 (m, 1H), 5.01 (d, J=9.6 Hz, 1H), 3.85-3.80 (m, 3H), 3.44-3.38 (m, 1H), 3.25-3.10 (m, 2H), 2.51-2.33 (m, 7H), 2.06-1.98 (m, 6H), 1.56-1.28 (m, 6H). MS (ESI) m/z: [M+H]⁺ Found 637.3.

### Example 7

### N—[(S)-(4,4-Difluorocyclohexyl)-[6-[(1R)-1-(4,4,4-trifluorobutanoylamino)ethyl]-1H-benzimidazol-2-yl]methyl]-2-ethyl-triazole-4-carboxamide

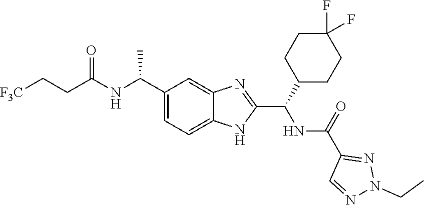 [SMILES: CCn1ncc(C(=O)N[C@H](c2nc3cc([C@@H](C)NC(=O)CCC(F)(F)F)ccc3[nH]2)C2CCC(F)(F)CC2)n1]

The title compound was prepared as described for the synthesis of Example 1, using 2-ethyl-2H-1,2,3-triazole-4-carboxylic acid in place of 1-(3,3,3-trifluoropropyl)-1H-pyrazole-4-carboxylic acid, and stirring at rt for 2 h followed by 40° C. for 1.5 h instead of stirring at rt for 3 h. The title compound was also further purified by basic preparative HPLC (NH₄OH). ¹H NMR (400 MHz, CDCl₃) δ 10.54-10.37 (m, 1H), 8.01 (s, 1H), 7.72-7.63 (m, 1H), 7.58-7.50 (m, 1H), 7.41-7.33 (m, 1H), 7.21 (t, J=9.1 Hz, 1H), 5.83-5.74 (m, 1H), 5.27-5.05 (m, 2H), 4.50-4.42 (m, 2H), 2.57-2.35 (m, 5H), 2.21-2.02 (m, 3H), 1.87-1.65 (m, 3H), 1.58-1.51 (m, 7H), 1.51-1.43 (m, 1H). MS (ESI) m/z: [M+H]⁺ Found 556.3.

### Example 8

### 2-(Cyclopropylmethyl)-N—[(S)-(4,4-difluorocyclohexyl)-[6-[(1R)-1-(4,4,4-trifluorobutanoylamino)ethyl]-1H-benzimidazol-2-yl]methyl]triazole-4-carboxamide

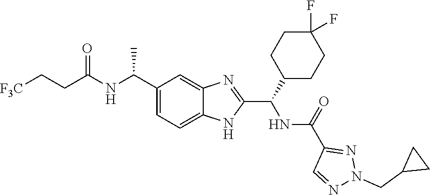 [SMILES: C[C@@H](NC(=O)CCC(F)(F)F)c1ccc2[nH]c([C@@H](NC(=O)c3cnn(CC4CC4)n3)C3CCC(F)(F)CC3)nc2c1]

The title compound was prepared as described for the synthesis of Example 1, using 2-(cyclopropylmethyl)-2H-1,2,3-triazole-4-carboxylic acid (Intermediate 10) in place of 1-(3,3,3-trifluoropropyl)-1H-pyrazole-4-carboxylic acid, and stirring at rt for 2 h followed by 40° C. for 3 h and then rt overnight instead of stirring at rt for 3 h. ¹H NMR (500 MHz, CDCl₃) δ 11.67-11.46 (m, 1H), 8.00-7.95 (m, 1H), 7.91 (t, J=8.0 Hz, 1H), 7.65-7.58 (m, 1H), 7.29-7.23 (m, 1H), 7.19-7.13 (m, 1H), 6.07 (dd, J=48.3, 7.6 Hz, 1H), 5.26-5.12 (m, 2H), 4.24-4.15 (m, 2H), 2.49-2.32 (m, 5H), 2.17-2.09 (m, 2H), 2.03 (br s, 1H), 1.81-1.59 (m, 3H), 1.49 (dd, J=28.3, 6.9 Hz, 4H), 1.44-1.28 (m, 2H), 0.65-0.57 (m, 2H), 0.43-0.35 (m, 2H). MS (ESI) m/z: [M+H]⁺ Found 582.3.

### Example 9

### 3-(Cyclopropylmethyl)-N—[(S)-(4,4-difluorocyclohexyl)-[6-[(1R)-1-(4,4,4-trifluorobutanoylamino)ethyl]-1H-benzimidazol-2-yl]methyl]triazole-4-carboxamide

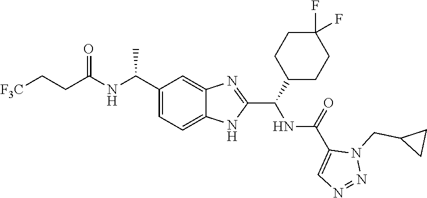 [SMILES: C[C@@H](NC(=O)CCC(F)(F)F)c1ccc2[nH]c([C@@H](NC(=O)c3cnnn3CC3CC3)C3CCC(F)(F)CC3)nc2c1]

The title compound was prepared as described for the synthesis of Example 1, using 1-(cyclopropylmethyl)-1H-1,2,3-triazole-5-carboxylic acid (Intermediate 12) in place of 1-(3,3,3-trifluoropropyl)-1H-pyrazole-4-carboxylic acid, and stirring at rt for 2 h followed by 40° C. for 3 h and then rt overnight instead of stirring at rt for 3 h. ¹H NMR (500 MHz, CDCl₃) δ 11.74-11.64 (m, 1H), 8.79-8.71 (m, 1H), 8.21 (s, 1H), 7.63-7.55 (m, 1H), 7.40-7.34 (m, 1H), 7.24-7.16 (m, 1H), 6.59 (dd, J=23.6, 7.7 Hz, 1H), 5.22-5.12 (m, 1H), 5.12-5.03 (m, 1H), 4.55 (d, J=7.3 Hz, 2H), 2.49-2.37 (m, 4H), 2.23-2.00 (m, 4H), 1.83-1.60 (m, 2H), 1.54-1.33 (m, 7H), 0.56-0.48 (m, 2H), 0.48-0.38 (m, 2H). MS (ESI) m/z: [M+H]⁺ Found 582.3.

### Example 10

### 1-(Cyclopropylmethyl)-N—[(S)-(4,4-difluorocyclohexyl)-[6-[(1R)-1-(4,4,4-trifluorobutanoylamino)ethyl]-1H-benzimidazol-2-yl]methyl]triazole-4-carboxamide

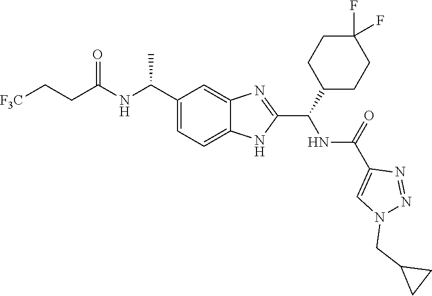 [SMILES: C[C@@H](NC(=O)CCC(F)(F)F)c1ccc2[nH]c([C@@H](NC(=O)c3cn(CC4CC4)nn3)C3CCC(F)(F)CC3)nc2c1]

The title compound was prepared as described for the synthesis of Example 1, using 1-(cyclopropylmethyl)-1H-1,2,3-triazole-4-carboxylic acid (Intermediate 14) in place of 1-(3,3,3-trifluoropropyl)-1H-pyrazole-4-carboxylic acid, and stirring at rt for 2 h followed by 40° C. for 3 h and then rt overnight instead of stirring at rt for 3 h. ¹H NMR (500 MHz, CDCl₃) δ 11.63-11.20 (m, 1H), 8.30-8.14 (m, 2H), 7.69-7.59 (m, 1H), 7.33-7.27 (m, 1H), 7.19-7.13 (m, 1H), 6.28 (dd, J=17.0, 7.7 Hz, 1H), 5.28-5.10 (m, 2H), 4.21 (d, J=7.3 Hz, 2H), 2.52-2.35 (m, 5H), 2.08-1.96 (m, 3H), 1.75-1.58 (m, 3H), 1.53-1.35 (m, 5H), 1.30-1.21 (m, 1H), 0.73-0.62 (m, 2H), 0.46-0.38 (m, 2H). MS (ESI) m/z: [M+H]⁺ Found 582.2.

### Example 11

### N—[(S)-(4,4-Difluorocyclohexyl)-[6-[(1R)-1-(4,4,4-trifluorobutanoylamino)ethyl]-1H-benzimidazol-2-yl]methyl]-1-isopentyl-5-isopropyl-pyrazole-4-carboxamide

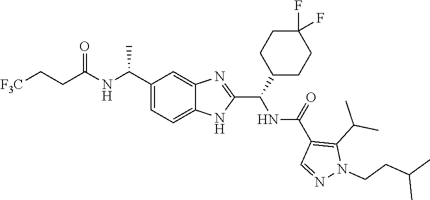 [SMILES: CC(C)CCn1ncc(C(=O)N[C@H](c2nc3cc([C@@H](C)NC(=O)CCC(F)(F)F)ccc3[nH]2)C2CCC(F)(F)CC2)c1C(C)C]

The title compound was prepared as described for the synthesis of Example 1, using 1-(3-methylbutyl)-5-(propan-2-yl)-1H-pyrazole-4-carboxylic acid in place of 1-(3,3,3-trifluoropropyl)-1H-pyrazole-4-carboxylic acid, and stirring at rt for 2 h followed by 40° C. for 1 h and then rt overnight instead of stirring at rt for 3 h. The title compound was also further purified by basic preparative HPLC (NH₄OH). ¹H NMR (500 MHz, CDCl₃) δ 11.18-10.90 (m, 1H), 7.69 (d, J=5.5 Hz, 1H), 7.66-7.62 (m, 1H), 7.37-7.29 (m, 1H), 7.24-7.16 (m, 2H), 5.91 (dd, J=35.9, 7.8 Hz, 1H), 5.27-5.15 (m, 1H), 5.01-4.90 (m, 1H), 4.15-4.07 (m, 2H), 3.60-3.46 (m, 1H), 2.50-2.33 (m, 5H), 2.13-2.01 (m, 2H), 1.72-1.61 (m, 8H), 1.53 (d, J=6.9 Hz, 3H), 1.41-1.36 (m, 6H), 1.35-1.24 (m, 1H), 0.96 (d, J=6.5 Hz, 6H). MS (ESI) m/z: [M+H]⁺ Found 639.2.

### Example 12

### 2-(2-Cyclopropylethyl)-N—[(S)-(4,4-difluorocyclohexyl)-[6-[(1R)-1-(4,4,4-trifluorobutanoylamino)ethyl]-1H-benzimidazol-2-yl]methyl]triazole-4-carboxamide

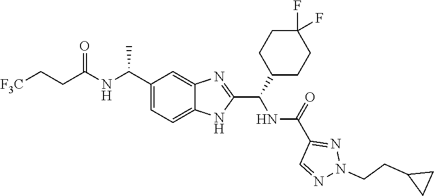 [SMILES: C[C@@H](NC(=O)CCC(F)(F)F)c1ccc2[nH]c([C@@H](NC(=O)c3cnn(CCC4CC4)n3)C3CCC(F)(F)CC3)nc2c1]

The title compound was prepared as described for the synthesis of Example 1, using 2-(2-cyclopropylethyl)-2H-1,2,3-triazole-4-carboxylic acid (Intermediate 16) in place of 1-(3,3,3-trifluoropropyl)-1H-pyrazole-4-carboxylic acid, and stirring at rt for 2 h followed by 40° C. for 1.5 h and then rt overnight instead of stirring at rt for 3 h. ¹H NMR (500 MHz, CDCl₃) δ 11.48 (d, J=58.7 Hz, 1H), 7.98 (s, 1H), 7.87 (t, J=8.5 Hz, 1H), 7.65-7.59 (m, 1H), 7.31-7.24 (m, 1H), 7.20-7.15 (m, 1H), 6.04 (dd, J=44.9, 7.6 Hz, 1H), 5.25-5.12 (m, 2H), 4.48-4.42 (m, 2H), 2.49-2.35 (m, 5H), 2.17-1.60 (m, 10H), 1.54-1.39 (m, 5H), 0.66-0.56 (m, 1H), 0.44-0.40 (m, 2H). MS (ESI) m/z: [M+H]⁺ Found 596.2.

### Example 13

### 3-(2-Cyclopropylethyl)-N—[(S)-(4,4-difluorocyclohexyl)-[6-[(1R)-1-(4,4,4-trifluorobutanoylamino)ethyl]-1H-benzimidazol-2-yl]methyl]triazole-4-carboxamide

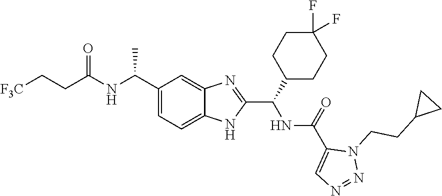 [SMILES: C[C@@H](NC(=O)CCC(F)(F)F)c1ccc2[nH]c([C@@H](NC(=O)c3cnnn3CCC3CC3)C3CCC(F)(F)CC3)nc2c1]

The title compound was prepared as described for the synthesis of Example 1, using 1-(2-cyclopropylethyl)-1H-1,2,3-triazole-5-carboxylic acid (Intermediate 18) in place of 1-(3,3,3-trifluoropropyl)-1H-pyrazole-4-carboxylic acid, and stirring at rt for 2 h followed by 40° C. for 17.5 h instead of stirring at rt for 3 h. ¹H NMR (500 MHz, CDCl₃) δ 11.80-11.67 (m, 1H), 8.76-8.63 (m, 1H), 8.23-8.14 (m, 1H), 7.63-7.56 (m, 1H), 7.43-7.34 (m, 1H), 7.22 (t, J=9.4 Hz, 1H), 6.63 (dd, J=19.6, 7.7 Hz, 1H), 5.24-5.12 (m, 1H), 5.09-5.01 (m, 1H), 4.79 (t, J=7.2 Hz, 2H), 2.63-2.41 (m, 4H), 2.24-2.10 (m, 2H), 2.08-1.97 (m, 2H), 1.80-1.63 (m, 4H), 1.55-1.45 (m, 5H), 1.40-1.28 (m, 1H), 0.67-0.57 (m, 1H), 0.41-0.31 (m, 2H), −0.03-−0.10 (m, 2H). MS (ESI) m/z: [M+H]⁺ Found 596.1.

### Example 14

### N—[(S)-(4,4-Difluorocyclohexyl)-[6-[(1R)-1-(4,4,4-trifluorobutanoylamino)ethyl]-1H-benzimidazol-2-yl]methyl]-2-(2-methoxyethyl)triazole-4-carboxamide

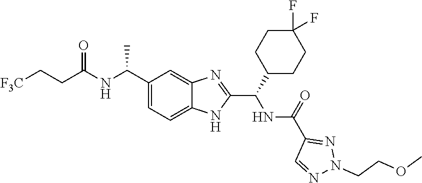 [SMILES: COCCn1ncc(C(=O)N[C@H](c2nc3cc([C@@H](C)NC(=O)CCC(F)(F)F)ccc3[nH]2)C2CCC(F)(F)CC2)n1]

The title compound was prepared as described for the synthesis of Example 1, using 2-(2-methoxyethyl)-2H-1,2,3-triazole-4-carboxylic acid (Intermediate 20) in place of 1-(3,3,3-trifluoropropyl)-1H-pyrazole-4-carboxylic acid, and stirring at rt for 2 h followed by 40° C. for 18 h instead of stirring at rt for 3 h. ¹H NMR (500 MHz, CDCl₃) δ 11.60 (d, J=55.1 Hz, 1H), 7.98 (s, 1H), 7.93 (t, J=9.2 Hz, 1H), 7.64-7.58 (m, 1H), 7.25-7.12 (m, 2H), 6.15 (dd, J=43.2, 7.7 Hz, 1H), 5.27-5.12 (m, 2H), 4.56-4.48 (m, 2H), 3.87-3.79 (m, 2H), 3.34-3.27 (m, 3H), 2.47-2.33 (m, 5H), 2.17-2.08 (m, 2H), 2.06-2.04 (m, 1H), 1.79-1.60 (m, 3H), 1.55-1.38 (m, 5H). MS (ESI) m/z: [M+H]⁺ Found 586.0.

### Example 15

### N—[(S)-(4,4-Difluorocyclohexyl)-[6-[(1R)-1-(4,4,4-trifluorobutanoylamino)ethyl]-1H-benzimidazol-2-yl]methyl]-3-(2-methoxyethyl)triazole-4-carboxamide

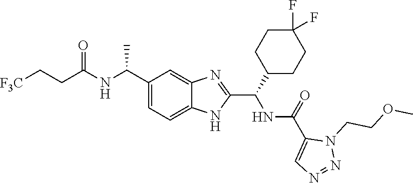 [SMILES: COCCn1nncc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 1, using 1-(2-methoxyethyl)-1H-1,2,3-triazole-5-carboxylic acid (Intermediate 22) in place of 1-(3,3,3-trifluoropropyl)-1H-pyrazole-4-carboxylic acid, and stirring at rt for 2 h followed by 40° C. for 18 h instead of stirring at rt for 3 h. ¹H NMR (500 MHz, CDCl₃) δ 8.15 (s, 1H), 7.62-7.56 (m, 1H), 7.40-7.34 (m, 1H), 7.25-7.18 (m, 1H), 6.79-6.70 (m, 1H), 5.22-5.13 (m, 1H), 5.11-5.06 (m, 1H), 4.92-4.87 (m, 2H), 3.79 (t, J=5.4 Hz, 2H), 3.25 (s, 3H), 2.55 (s, 3H), 2.48-2.37 (m, 4H), 2.26-2.10 (m, 2H), 1.82-1.62 (m, 2H), 1.56-1.45 (m, 5H), 1.40-1.31 (m, 1H). MS (ESI) m/z: [M+H]⁺ Found 586.0.

### Example 16

### N—[(S)-(4,4-Difluorocyclohexyl)-[6-[(1R)-1-(4,4,4-trifluorobutanoylamino)ethyl]-1H-benzimidazol-2-yl]methyl]-1-(2-methoxyethyl)triazole-4-carboxamide

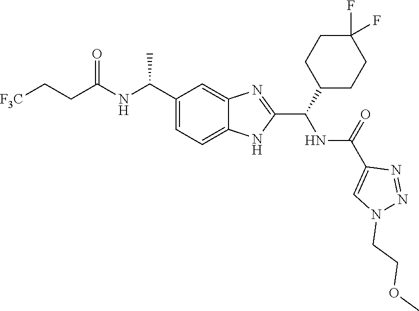 [SMILES: COCCn1cc(C(=O)N[C@H](c2nc3cc([C@@H](C)NC(=O)CCC(F)(F)F)ccc3[nH]2)C2CCC(F)(F)CC2)nn1]

The title compound was prepared as described for the synthesis of Example 1, using 1-(2-methoxyethyl)-1H-1,2,3-triazole-4-carboxylic acid (Intermediate 24) in place of 1-(3,3,3-trifluoropropyl)-1H-pyrazole-4-carboxylic acid, and stirring at rt for 2 h followed by 40° C. for 18 h instead of stirring at rt for 3 h. ¹H NMR (500 MHz, CDCl₃) δ 11.69-11.22 (m, 1H), 8.31-8.14 (m, 2H), 7.71-7.60 (m, 1H), 7.36-7.27 (m, 1H), 7.18-7.13 (m, 1H), 6.34 (dd, J=22.6, 7.8 Hz, 1H), 5.29-5.13 (m, 2H), 4.56-4.49 (m, 2H), 3.75-3.68 (m, 2H), 3.30 (s, 3H), 2.50-2.35 (m, 5H), 2.09-1.96 (m, 3H), 1.72-1.59 (m, 3H), 1.53-1.36 (m, 5H). MS (ESI) m/z: [M+H]⁺ Found 586.1.

### Example 17

### 1-(2-Cyanoethyl)-N—[(S)-(4,4-difluorocyclohexyl)-[6-[(1R)-1-(4,4,4-trifluorobutanoylamino)ethyl]-1H-benzimidazol-2-yl]methyl]-5-methyl-pyrazole-4-carboxamide

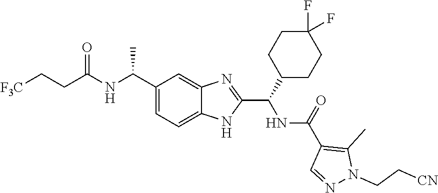 [SMILES: Cc1c(C(=O)N[C@H](c2nc3cc([C@@H](C)NC(=O)CCC(F)(F)F)ccc3[nH]2)C2CCC(F)(F)CC2)cnn1CCC#N]

The title compound was prepared as described for the synthesis of Example 1, using 1-(2-cyanoethyl)-5-methyl-1H-pyrazole-4-carboxylic acid in place of 1-(3,3,3-trifluoropropyl)-1H-pyrazole-4-carboxylic acid, and stirring at rt for 2.5 h followed by 40° C. for 2.5 h instead of stirring at rt for 3 h. The title compound was also further purified by basic preparative HPLC (NH₄OH). ¹H NMR (500 MHz, CDCl₃) δ 7.95 (d, J=3.0 Hz, 1H), 7.61-7.55 (m, 1H), 7.39-7.33 (m, 1H), 7.23-7.17 (m, 1H), 6.52-6.35 (m, 1H), 5.22-5.13 (m, 1H), 5.05 (dd, J=12.4, 9.6 Hz, 1H), 4.34-4.29 (m, 2H), 2.94 (t, J=6.6 Hz, 2H), 2.61 (d, J=2.0 Hz, 3H), 2.50-2.36 (m, 4H), 2.31-2.08 (m, 3H), 2.07-2.02 (m, 3H), 1.81-1.62 (m, 2H), 1.55-1.50 (m, 4H), 1.48-1.31 (m, 2H). MS (ESI) m/z: [M+H]⁺ Found 594.3.

### Example 18

### N—[(S)-(4,4-Difluorocyclohexyl)-[6-[(1R)-1-(4,4,4-trifluorobutanoylamino)ethyl]-1H-benzimidazol-2-yl]methyl]-5-methyl-1-(2,2,2-trifluoroethyl)pyrazole-4-carboxamide

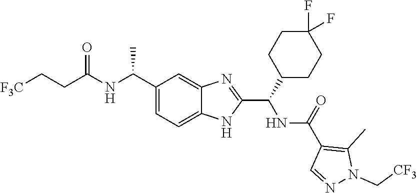 [SMILES: Cc1c(C(=O)N[C@H](c2nc3cc([C@@H](C)NC(=O)CCC(F)(F)F)ccc3[nH]2)C2CCC(F)(F)CC2)cnn1CC(F)(F)F]

The title compound was prepared as described for the synthesis of Example 1, using 5-methyl-1-(2,2,2-trifluoroethyl)-1H-pyrazole-4-carboxylic acid in place of 1-(3,3,3-trifluoropropyl)-1H-pyrazole-4-carboxylic acid, and stirring at rt for 2.5 h followed by 40° C. for 1.5 h instead of stirring at rt for 3 h. The title compound was also further purified by basic preparative HPLC (NH₄OH). ¹H NMR (500 MHz, CDCl₃) δ 8.01-7.97 (m, 1H), 7.63-7.57 (m, 1H), 7.42-7.37 (m, 1H), 7.30 (s, 1H), 7.26-7.18 (m, 1H), 6.69-6.56 (m, 1H), 5.19 (q, J=7.2 Hz, 1H), 5.06-4.99 (m, 1H), 4.72-4.63 (m, 2H), 3.43-3.38 (m, 1H), 2.60-2.56 (m, 3H), 2.50-2.40 (m, 4H), 2.25-2.10 (m, 2H), 2.10-1.98 (m, 2H), 1.83-1.60 (m, 2H), 1.55-1.30 (m, 6H). MS (ESI) m/z: [M+H]⁺ Found 623.3.

### Example 19

### N—[(S)-(4,4-Difluorocyclohexyl)-[6-[(1R)-1-(4,4,4-trifluorobutanoylamino)ethyl]-1H-benzimidazol-2-yl]methyl]-2-(4,4,4-trifluorobutyl)triazole-4-carboxamide

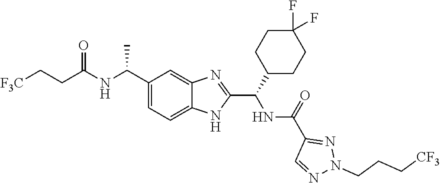 [SMILES: C[C@@H](NC(=O)CCC(F)(F)F)c1ccc2[nH]c([C@@H](NC(=O)c3cnn(CCCC(F)(F)F)n3)C3CCC(F)(F)CC3)nc2c1]

The title compound was prepared as described for the synthesis of Example 1, using 2-(4,4,4-trifluorobutyl)-2H-1,2,3-triazole-4-carboxylic acid (Intermediate 26) in place of 1-(3,3,3-trifluoropropyl)-1H-pyrazole-4-carboxylic acid, and stirring at rt for 2 h followed by 40° C. for 17 h instead of stirring at rt for 3 h. ¹H NMR (500 MHz, CDCl₃) δ 11.59-11.37 (m, 1H), 8.02-7.97 (m, 1H), 7.92 (dd, J=13.5, 8.7 Hz, 1H), 7.65-7.58 (m, 1H), 7.30-7.24 (m, 1H), 7.19-7.14 (m, 1H), 6.07 (dd, J=48.1, 7.6 Hz, 1H), 5.23-5.12 (m, 2H), 4.48-4.42 (m, 2H), 2.48-2.31 (m, 5H), 2.22-2.17 (m, 2H), 2.14-1.99 (m, 5H), 1.78-1.59 (m, 3H), 1.53-1.46 (m, 4H), 1.45-1.37 (m, 1H). MS (ESI) m/z: [M+H]⁺ Found 638.1.

### Example 20

### N—[(S)-(4,4-Difluorocyclohexyl)-[6-[(1R)-1-(4,4,4-trifluorobutanoylamino)ethyl]-1H-benzimidazol-2-yl]methyl]-3-(4,4,4-trifluorobutyl)triazole-4-carboxamide

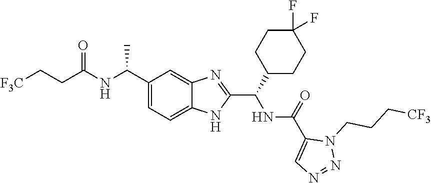 [SMILES: C[C@@H](NC(=O)CCC(F)(F)F)c1ccc2[nH]c([C@@H](NC(=O)c3cnnn3CCCC(F)(F)F)C3CCC(F)(F)CC3)nc2c1]

The title compound was prepared as described for the synthesis of Example 1, using 1-(4,4,4-trifluorobutyl)-1H-1,2,3-triazole-5-carboxylic acid (Intermediate 28) in place of 1-(3,3,3-trifluoropropyl)-1H-pyrazole-4-carboxylic acid, and stirring at rt for 2 h followed by 40° C. for 17 h instead of stirring at rt for 3 h. ¹H NMR (500 MHz, CDCl₃) δ 11.85-11.70 (m, 1H), 8.87-8.72 (m, 1H), 8.24 (s, 1H), 7.63-7.54 (m, 1H), 7.42-7.35 (m, 1H), 7.25-7.17 (m, 1H), 6.75-6.66 (m, 1H), 5.23-5.11 (m, 1H), 5.11-5.04 (m, 1H), 4.81-4.72 (m, 2H), 2.74-2.51 (m, 4H), 2.51-2.36 (m, 4H), 2.21-2.17 (m, 2H), 2.08-1.99 (m, 2H), 1.82-1.60 (m, 2H), 1.53-1.32 (m, 6H). MS (ESI) m/z: [M+H]⁺ Found 638.1.

### Example 21

### N—[(S)-(4,4-Difluorocyclohexyl)-[6-[(1R)-1-(4,4,4-trifluorobutanoylamino)ethyl]-1H-benzimidazol-2-yl]methyl]-1-(4,4,4-trifluorobutyl)triazole-4-carboxamide

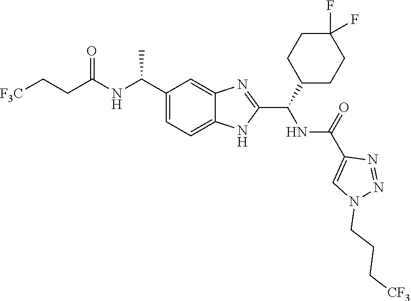 [SMILES: C[C@@H](NC(=O)CCC(F)(F)F)c1ccc2[nH]c([C@@H](NC(=O)c3cn(CCCC(F)(F)F)nn3)C3CCC(F)(F)CC3)nc2c1]

The title compound was prepared as described for the synthesis of Example 1, using 1-(4,4,4-trifluorobutyl)-1H-1,2,3-triazole-4-carboxylic acid (Intermediate 30) in place of 1-(3,3,3-trifluoropropyl)-1H-pyrazole-4-carboxylic acid, and stirring at rt for 2 h followed by 40° C. for 17 h instead of stirring at rt for 3 h. ¹H NMR (500 MHz, DMSO-d₆) δ 12.34 (s, 1H), 8.67 (d, J=9.1 Hz, 1H), 8.47-8.38 (m, 1H), 7.56-7.46 (m, 1H), 7.45-7.32 (m, 1H), 7.16-7.07 (m, 1H), 5.21 (t, J=8.7 Hz, 1H), 5.05-4.97 (m, 1H), 4.51 (t, J=7.0 Hz, 2H), 3.85-3.52 (m, 1H), 3.30-3.26 (m, 2H), 2.47-2.36 (m, 3H), 2.32-2.25 (m, 2H), 2.24-2.20 (m, 1H), 2.12-2.06 (m, 2H), 1.93-1.88 (m, 1H), 1.83-1.74 (m, 2H), 1.56-1.48 (m, 1H), 1.39-1.23 (m, 5H), 1.12 (d, J=6.1 Hz, 1H). MS (ESI) m/z: [M+H]⁺ Found 638.1.

### Example 22

### N—[(S)-(4,4-Difluorocyclohexyl)-[6-[(1R)-1-(4,4,4-trifluorobutanoylamino)ethyl]-1H-benzimidazol-2-yl]methyl]-2-(2,2,2-trifluoroethyl)triazole-4-carboxamide

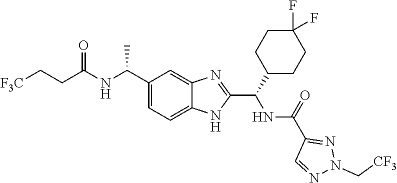 [SMILES: C[C@@H](NC(=O)CCC(F)(F)F)c1ccc2[nH]c([C@@H](NC(=O)c3cnn(CC(F)(F)F)n3)C3CCC(F)(F)CC3)nc2c1]

The title compound was prepared as described for the synthesis of Example 1, using 2-(2,2,2-trifluoroethyl)-2H-1,2,3-triazole-4-carboxylic acid (Intermediate 32) in place of 1-(3,3,3-trifluoropropyl)-1H-pyrazole-4-carboxylic acid, and stirring at rt for 2 h followed by 40° C. for 16 h instead of stirring at rt for 3 h. ¹H NMR (500 MHz, CDCl₃) δ 11.81-11.65 (m, 1H), 8.11-8.03 (m, 1H), 8.01-7.96 (m, 1H), 7.61-7.55 (m, 1H), 7.23-7.12 (m, 2H), 6.12 (dd, J=74.0, 7.6 Hz, 1H), 5.22-5.09 (m, 2H), 4.94-4.84 (m, 2H), 2.50-2.28 (m, 5H), 2.07-1.97 (m, 1H), 1.92-1.57 (m, 4H), 1.56-1.37 (m, 6H). MS (ESI) m/z: [M+H]⁺ Found 610.1.

### Example 23

### N—[(S)-(4,4-Difluorocyclohexyl)-[6-[(1R)-1-(4,4,4-trifluorobutanoylamino)ethyl]-1H-benzimidazol-2-yl]methyl]-3-(2,2,2-trifluoroethyl)triazole-4-carboxamide

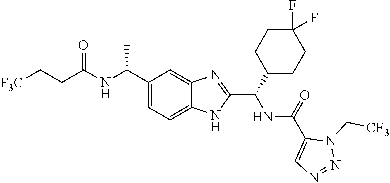 [SMILES: C[C@@H](NC(=O)CCC(F)(F)F)c1ccc2[nH]c([C@@H](NC(=O)c3cnnn3CC(F)(F)F)C3CCC(F)(F)CC3)nc2c1]

The title compound was prepared as described for the synthesis of Example 1, using 1-(2,2,2-trifluoroethyl)-1H-1,2,3-triazole-5-carboxylic acid (Intermediate 34) in place of 1-(3,3,3-trifluoropropyl)-1H-pyrazole-4-carboxylic acid, and stirring at rt for 2 h followed by 40° C. for 16 h instead of stirring at rt for 3 h. ¹H NMR (500 MHz, CDCl₃) δ 11.78-11.62 (m, 1H), 9.02-8.88 (m, 1H), 8.32-8.23 (m, 1H), 7.62-7.56 (m, 1H), 7.41-7.35 (m, 1H), 7.25-7.18 (m, 1H), 6.59-6.51 (m, 1H), 5.60-5.41 (m, 2H), 5.18 (dq, J=13.9, 6.9 Hz, 1H), 5.06 (dd, J=9.6, 3.8 Hz, 1H), 2.51-2.38 (m, 4H), 2.28 (s, 3H), 2.23-2.11 (m, 2H), 1.79-1.61 (m, 2H), 1.53 (dd, J=7.0, 3.0 Hz, 3H), 1.49-1.44 (m, 1H), 1.42-1.29 (m, 1H). MS (ESI) m/z: [M+H]⁺ Found 610.0.

### Example 24

### N—[(S)-(4,4-Difluorocyclohexyl)-[6-[(1R)-1-(4,4,4-trifluorobutanoylamino)ethyl]-1H-benzimidazol-2-yl]methyl]-1-(2,2,2-trifluoroethyl)triazole-4-carboxamide

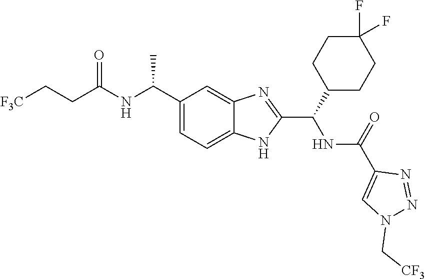 [SMILES: C[C@@H](NC(=O)CCC(F)(F)F)c1ccc2[nH]c([C@@H](NC(=O)c3cn(CC(F)(F)F)nn3)C3CCC(F)(F)CC3)nc2c1]

The title compound was prepared as described for the synthesis of Example 1, using 1-(2,2,2-trifluoroethyl)-1H-1,2,3-triazole-4-carboxylic acid (Intermediate 36) in place of 1-(3,3,3-trifluoropropyl)-1H-pyrazole-4-carboxylic acid, and stirring at rt for 2 h followed by 40° C. for 16 h instead of stirring at rt for 3 h. ¹H NMR (500 MHz, CD₃OD) δ 8.56 (s, 1H), 7.51 (s, 2H), 7.23 (dd, J=8.4, 1.6 Hz, 1H), 5.39 (q, J=8.6 Hz, 2H), 5.29 (d, J=8.6 Hz, 1H), 5.11 (q, J=7.0 Hz, 1H), 2.53-2.40 (m, 4H), 2.32-2.23 (m, 1H), 2.11-1.99 (m, 3H), 1.89-1.70 (m, 2H), 1.60-1.51 (m, 2H), 1.49 (d, J=7.0 Hz, 3H), 1.44-1.36 (m, 1H). MS (ESI) m/z: [M+H]⁺ Found 610.0.

### Example 25

### N—[(S)-(4,4-Difluorocyclohexyl)-[6-[(1R)-1-(4,4,4-trifluorobutanoylamino)ethyl]-1H-benzimidazol-2-yl]methyl]-3-methyl-1H-pyrazole-4-carboxamide

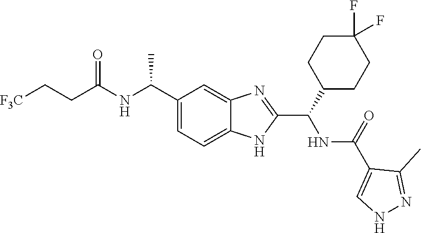 [SMILES: Cc1n[nH]cc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 1, using 3-methylpyrazole-4-carboxylic acid in place of 1-(3,3,3-trifluoropropyl)-1H-pyrazole-4-carboxylic acid, and the reaction was stirred at rt for 19 h instead of 3 h. The title compound was also further purified by basic preparative HPLC (NH₄OH). ¹H NMR (500 MHz, CDCl₃) δ 7.94 (s, 1H), 7.63-7.56 (m, 1H), 7.42-7.37 (m, 1H), 7.24-7.18 (m, 1H), 6.48 (s, 1H), 5.24-5.14 (m, 1H), 5.04 (d, J=9.5 Hz, 1H), 3.44-3.39 (m, 1H), 2.52-2.51 (m, 3H), 2.48-2.38 (m, 4H), 2.25-2.18 (m, 1H), 2.11-2.09 (m, 2H), 1.96-1.92 (m, 3H), 1.82-1.64 (m, 2H), 1.55-1.51 (m, 4H), 1.50-1.43 (m, 1H), 1.40-1.32 (m, 1H). MS (ESI) m/z: [M+H]⁺ Found 541.1.

### Example 26

### N—[(S)-(4,4-Difluorocyclohexyl)-[6-[(1R)-1-(4,4,4-trifluorobutanoylamino)ethyl]-1H-benzimidazol-2-yl]methyl]-1-[2-(trifluoromethoxy)ethyl]pyrazole-4-carboxamide

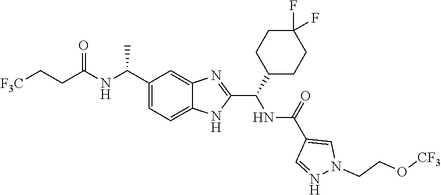 [SMILES: C[C@@H](NC(=O)CCC(F)(F)F)c1ccc2[nH]c([C@@H](NC(=O)c3cnn(CCOC(F)(F)F)c3)C3CCC(F)(F)CC3)nc2c1]

The title compound was prepared as described for the synthesis of Example 1, using 1-(2-(trifluoromethoxy)ethyl)-1H-pyrazole-4-carboxylic acid (Intermediate 38) in place of 1-(3,3,3-trifluoropropyl)-1H-pyrazole-4-carboxylic acid, and the reaction was stirred at rt for 15.5 h instead of 3 h. The title compound was also further purified by basic preparative HPLC (NH₄OH). ¹H NMR (500 MHz, CDCl₃) δ 7.99 (s, 1H), 7.95 (s, 1H), 7.58-7.52 (m, 1H), 7.38-7.30 (m, 1H), 7.21-7.14 (m, 1H), 6.57-6.50 (m, 1H), 5.22-5.08 (m, 1H), 5.02 (d, J=9.7 Hz, 1H), 4.36 (q, J=4.4, 3.7 Hz, 2H), 4.30 (dd, J=5.2, 4.2 Hz, 2H), 3.41-3.34 (m, 1H), 2.46-2.36 (m, 4H), 2.17-1.93 (m, 4H), 1.78-1.56 (m, 2H), 1.53-1.27 (m, 6H). MS (ESI) m/z: [M+H]⁺ Found 639.0.

### Example 27

### N—[(S)-(4,4-Difluorocyclohexyl)-[6-[(1R)-1-(4,4,4-trifluorobutanoylamino)ethyl]-1H-benzimidazol-2-yl]methyl]-1-[2-(difluoromethoxy)ethyl]pyrazole-4-carboxamide

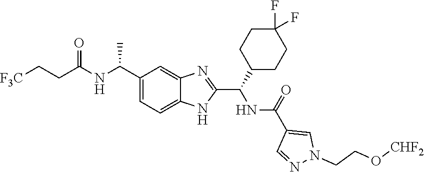 [SMILES: C[C@@H](NC(=O)CCC(F)(F)F)c1ccc2[nH]c([C@@H](NC(=O)c3cnn(CCOC(F)F)c3)C3CCC(F)(F)CC3)nc2c1]

The title compound was prepared as described for the synthesis of Example 1, using 1-(2-(difluoromethoxy)ethyl)-1H-pyrazole-4-carboxylic acid (Intermediate 40) in place of 1-(3,3,3-trifluoropropyl)-1H-pyrazole-4-carboxylic acid, and the reaction was stirred at rt for 4 h instead 3 h. The title compound was purified by basic preparative HPLC (NH₄OH). ¹H NMR (500 MHz, CDCl₃) δ 8.02 (d, J=3.2 Hz, 1H), 7.99-7.94 (m, 1H), 7.61-7.56 (m, 1H), 7.42-7.36 (m, 1H), 7.24-7.18 (m, 1H), 6.66-6.58 (m, 1H), 6.17 (t, J=73.6 Hz, 1H), 5.23-5.14 (m, 1H), 5.04 (d, J=9.7 Hz, 1H), 4.36 (t, J=5.1 Hz, 2H), 4.22 (t, J=5.1 Hz, 2H), 3.44-3.38 (m, 1H), 2.50-2.39 (m, 4H), 2.24-2.00 (m, 4H), 1.81-1.61 (m, 2H), 1.54-1.30 (m, 6H). MS (ESI) m/z: [M+H]⁺ Found 621.0.

### Example 28

### N—[(S)-(4,4-Difluorocyclohexyl)-[6-[(1R)-1-(4,4,4-trifluorobutanoylamino)ethyl]-1H-benzimidazol-2-yl]methyl]-1-(2,2-difluoroethyl)-5-methyl-pyrazole-4-carboxamide

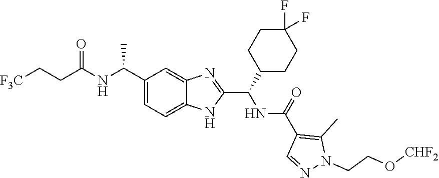 [SMILES: Cc1c(C(=O)N[C@H](c2nc3cc([C@@H](C)NC(=O)CCC(F)(F)F)ccc3[nH]2)C2CCC(F)(F)CC2)cnn1CCOC(F)F]

The title compound was prepared as described for the synthesis of Example 1, using 1-(2,2-difluoroethyl)-5-methyl-1H-pyrazole-4-carboxylic acid in place of 1-(3,3,3-trifluoropropyl)-1H-pyrazole-4-carboxylic acid, and the reaction was stirred at rt for 24 h instead of 3 h. ¹H NMR (500 MHz, CDCl₃) δ 7.92 (d, J=2.5 Hz, 1H), 7.58-7.47 (m, 1H), 7.35-7.29 (m, 1H), 7.15 (d, J=8.4 Hz, 1H), 6.91-6.77 (m, 1H), 6.16-5.88 (m, 1H), 5.16-5.07 (m, 1H), 5.01 (dd, J=9.6, 2.5 Hz, 1H), 4.41-4.30 (m, 2H), 3.39-3.30 (m, 1H), 2.85 (d, J=2.2 Hz, 4H), 2.44-2.32 (m, 4H), 2.19-1.94 (m, 4H), 1.77-1.56 (m, 2H), 1.49-1.25 (m, 6H). MS (ESI) m/z: [M+H]⁺ Found 605.1.

### Example 29

### 1-(Cyclopropylmethyl)-N—[(S)-(4,4-difluorocyclohexyl)-[6-[(1R)-1-(4,4,4-trifluorobutanoylamino)ethyl]-1H-benzimidazol-2-yl]methyl]-1,2,4-triazole-3-carboxamide

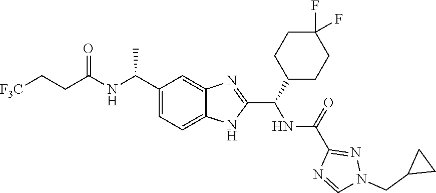 [SMILES: C[C@@H](NC(=O)CCC(F)(F)F)c1ccc2[nH]c([C@@H](NC(=O)c3ncn(CC4CC4)n3)C3CCC(F)(F)CC3)nc2c1]

The title compound was prepared as described for the synthesis of Example 1, using 1-(cyclopropylmethyl)-1H-1,2,4-triazole-3-carboxylic acid (Intermediate 42) in place of 1-(3,3,3-trifluoropropyl)-1H-pyrazole-4-carboxylic acid, and the reaction was stirred at rt for 21 h instead 3 h. The title compound was purified by basic preparative HPLC (NH₄OH). ¹H NMR (400 MHz, CDCl₃) δ 11.37 (d, J=67.0 Hz, 1H), 8.22 (d, J=6.6 Hz, 1H), 7.96 (dd, J=11.9, 8.9 Hz, 1H), 7.68-7.62 (m, 1H), 7.54-7.44 (m, 1H), 7.21-7.13 (m, 1H), 5.89 (dd, J=17.0, 7.8 Hz, 1H), 5.42 (td, J=9.1, 5.2 Hz, 1H), 5.19 (dq, J=14.5, 7.2 Hz, 1H), 4.09-4.02 (m, 2H), 2.55-2.34 (m, 5H), 2.21-1.99 (m, 3H), 1.85-1.68 (m, 3H), 1.62-1.43 (m, 5H), 1.37-1.28 (m, 1H), 0.77-0.67 (m, 2H), 0.43 (dd, J=5.7, 4.3 Hz, 2H). MS (ESI) m/z: [M+H]⁺ Found 582.2.

### Example 30

### 2-(Cyclopropylmethyl)-N—[(S)-(4,4-difluorocyclohexyl)-[6-[(1R)-1-(4,4,4-trifluorobutanoylamino)ethyl]-1H-benzimidazol-2-yl]methyl]-1,2,4-triazole-3-carboxamide

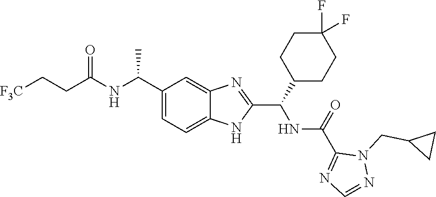 [SMILES: C[C@@H](NC(=O)CCC(F)(F)F)c1ccc2[nH]c([C@@H](NC(=O)c3ncnn3CC3CC3)C3CCC(F)(F)CC3)nc2c1]

The title compound was prepared as described for the synthesis of Example 1, using 1-(cyclopropylmethyl)-1H-1,2,4-triazole-5-carboxylic acid (Intermediate 44) in place of 1-(3,3,3-trifluoropropyl)-1H-pyrazole-4-carboxylic acid, and stirring at rt for 16 h followed by 40° C. for 23 h instead of stirring at rt for 3 h. The title compound was purified by basic preparative HPLC (NH₄OH). ¹H NMR (400 MHz, CDCl₃) δ 10.08 (d, J=64.1 Hz, 1H), 8.21 (dd, J=30.5, 8.8 Hz, 1H), 7.89-7.79 (m, 1H), 7.70-7.61 (m, 1H), 7.35-7.29 (m, 1H), 7.22-7.16 (m, 1H), 5.89-5.81 (m, 1H), 5.25-4.98 (m, 2H), 4.56-4.45 (m, 2H), 2.55-2.37 (m, 5H), 2.21-1.98 (m, 3H), 1.85-1.71 (m, 2H), 1.57-1.35 (m, 6H), 0.59-0.52 (m, 2H), 0.49-0.40 (m, 2H). MS (ESI) m/z: [M+H]⁺ Found 582.2.

### Example 31

### N—[(S)-(4,4-Difluorocyclohexyl)-[6-[(1R)-1-(4,4,4-trifluorobutanoylamino)ethyl]-1H-benzimidazol-2-yl]methyl]-2-methyl-1,2,4-triazole-3-carboxamide

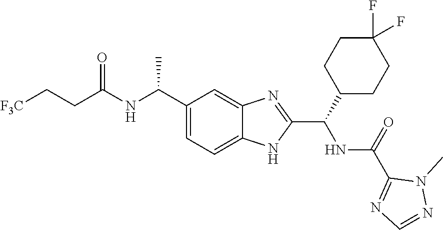 [SMILES: C[C@@H](NC(=O)CCC(F)(F)F)c1ccc2[nH]c([C@@H](NC(=O)c3ncnn3C)C3CCC(F)(F)CC3)nc2c1]

The title compound was prepared as described for the synthesis of Example 1, using 1-methyl-1H-1,2,4-triazole-5-carboxylic acid in place of 1-(3,3,3-trifluoropropyl)-1H-pyrazole-4-carboxylic acid, adding DMF (3 mL) to the reaction before stirring at rt, and stirring at rt for 2 h followed by 40° C. for 3 d instead of stirring at rt for 3 h. The title compound was purified by basic preparative HPLC (NH₄OH). ¹H NMR (400 MHz, CDCl₃) δ 10.06 (d, J=53.8 Hz, 1H), 8.17 (dd, J=33.6, 8.7 Hz, 1H), 7.82 (s, 1H), 7.69-7.61 (m, 1H), 7.36-7.29 (m, 1H), 7.23-7.17 (m, 1H), 5.87-5.77 (m, 1H), 5.20 (q, J=6.7 Hz, 1H), 5.04 (q, J=8.9 Hz, 1H), 4.25 (s, 3H), 2.53-2.38 (m, 5H), 2.20-2.01 (m, 3H), 1.84-1.68 (m, 3H), 1.56-1.39 (m, 5H). MS (ESI) m/z: [M+H]⁺ Found 542.1.

### Example 32

### N—[(S)-(4,4-Difluorocyclohexyl)-[6-[(1R)-1-(4,4,4-trifluorobutanoylamino)ethyl]-1H-benzimidazol-2-yl]methyl]-1-methyl-1,2,4-triazole-3-carboxamide

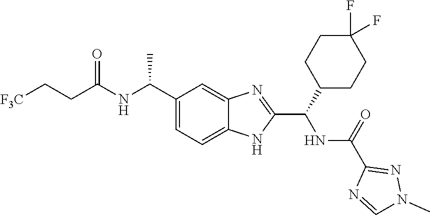 [SMILES: C[C@@H](NC(=O)CCC(F)(F)F)c1ccc2[nH]c([C@@H](NC(=O)c3ncn(C)n3)C3CCC(F)(F)CC3)nc2c1]

The title compound was prepared as described for the synthesis of Example 1, using 1-methyl-1H-1,2,4-triazole-3-carboxylic acid in place of 1-(3,3,3-trifluoropropyl)-1H-pyrazole-4-carboxylic acid, and stirring at rt for 2 h followed by 40° C. for 3 d instead of stirring at rt for 3 h. The title compound was purified by basic preparative HPLC (NH₄OH). ¹H NMR (400 MHz, CDCl₃) δ 11.35 (d, J=83.8 Hz, 1H), 8.09 (d, J=10.8 Hz, 1H), 8.00-7.90 (m, 1H), 7.69-7.61 (m, 1H), 7.52-7.40 (m, 1H), 7.20-7.12 (m, 1H), 5.89 (dd, J=16.1, 7.7 Hz, 1H), 5.47-5.36 (m, 1H), 5.25-5.10 (m, 1H), 3.99 (d, J=5.0 Hz, 3H), 2.51-2.33 (m, 5H), 2.22-2.04 (m, 3H), 1.84-1.68 (m, 3H), 1.61-1.45 (m, 5H). MS (ESI) m/z: [M+H]⁺ Found 542.1.

### Example 33

### N—[(S)-(4,4-Difluorocyclohexyl)-[6-[(1R)-1-(4,4,4-trifluorobutanoylamino)ethyl]-1H-benzimidazol-2-yl]methyl]-2-[2-(difluoromethoxy)ethyl]triazole-4-carboxamide

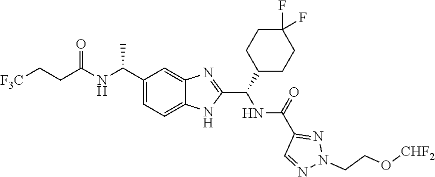 [SMILES: C[C@@H](NC(=O)CCC(F)(F)F)c1ccc2[nH]c([C@@H](NC(=O)c3cnn(CCOC(F)F)n3)C3CCC(F)(F)CC3)nc2c1]

The title compound was prepared as described for the synthesis of Example 1, using 2-(2-(difluoromethoxy)ethyl)-2H-1,2,3-triazole-4-carboxylic acid (Intermediate 46) in place of 1-(3,3,3-trifluoropropyl)-1H-pyrazole-4-carboxylic acid, and stirring at rt for 2 h followed by 40° C. for 18 h instead of stirring at rt for 3 h. The title compound was purified by basic preparative HPLC (NH₄OH). ¹H NMR (500 MHz, CDCl₃) δ 8.04-7.97 (m, 1H), 7.63-7.53 (m, 1H), 7.38-7.32 (m, 1H), 7.21-7.12 (m, 1H), 6.13 (td, J=73.3, 4.7 Hz, 1H), 5.17-5.08 (m, 1H), 5.05 (d, J=9.6 Hz, 1H), 4.69-4.59 (m, 2H), 4.36-4.30 (m, 2H), 2.42-2.35 (m, 4H), 2.27-2.05 (m, 4H), 2.02-1.97 (m, 2H), 1.77-1.62 (m, 2H), 1.54-1.41 (m, 6H), 1.36-1.29 (m, 1H). MS (ESI) m/z: [M+H]⁺ Found 622.0.

### Example 34

### N—[(S)-(4,4-Difluorocyclohexyl)-[6-[(1R)-1-(4,4,4-trifluorobutanoylamino)ethyl]-1H-benzimidazol-2-yl]methyl]-3-[2-(difluoromethoxy)ethyl]triazole-4-carboxamide

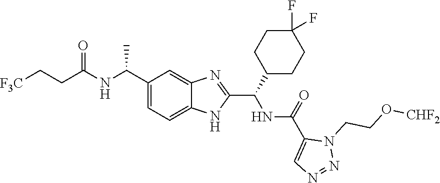 [SMILES: C[C@@H](NC(=O)CCC(F)(F)F)c1ccc2[nH]c([C@@H](NC(=O)c3cnnn3CCOC(F)F)C3CCC(F)(F)CC3)nc2c1]

The title compound was prepared as described for the synthesis of Example 1, using 1-(2-(difluoromethoxy)ethyl)-1H-1,2,3-triazole-5-carboxylic acid (Intermediate 48) in place of 1-(3,3,3-trifluoropropyl)-1H-pyrazole-4-carboxylic acid, and stirring at rt for 2 h followed by 40° C. for 18 h instead of stirring at rt for 3 h. The title compound was purified by basic preparative HPLC (NH₄OH). ¹H NMR (500 MHz, CDCl₃) δ 8.20-8.16 (m, 1H), 7.59-7.51 (m, 1H), 7.38-7.32 (m, 1H), 7.22-7.16 (m, 1H), 6.87-6.79 (m, 1H), 6.02 (td, J=73.6, 1.6 Hz, 1H), 5.17-5.08 (m, 1H), 5.03-4.97 (m, 1H), 4.97-4.89 (m, 2H), 4.25-4.18 (m, 2H), 3.38-3.32 (m, 1H), 2.70-2.65 (m, 1H), 2.45-2.33 (m, 4H), 2.18-1.94 (m, 4H), 1.77-1.60 (m, 2H), 1.49-1.28 (m, 6H). MS (ESI) m/z: [M+H]⁺ Found 622.0.

### Example 35

### N—[(S)-(4,4-Difluorocyclohexyl)-[6-[(1R)-1-(4,4,4-trifluorobutanoylamino)ethyl]-1H-benzimidazol-2-yl]methyl]-2-[2-(trifluoromethoxy)ethyl]triazole-4-carboxamide

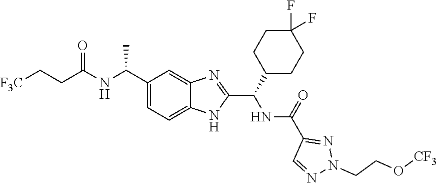 [SMILES: C[C@@H](NC(=O)CCC(F)(F)F)c1ccc2[nH]c([C@@H](NC(=O)c3cnn(CCOC(F)(F)F)n3)C3CCC(F)(F)CC3)nc2c1]

The title compound was prepared as described for the synthesis of Example 1, using 2-(2-(trifluoromethoxy)ethyl)-2H-1,2,3-triazole-4-carboxylic acid (Intermediate 50) in place of 1-(3,3,3-trifluoropropyl)-1H-pyrazole-4-carboxylic acid, and stirring at rt for 2 h followed by 40° C. for 20 h instead of stirring at rt for 3 h. The title compound was purified by basic preparative HPLC (NH₄OH). ¹H NMR (500 MHz, CDCl₃) δ 10.63 (d, J=69.9 Hz, 1H), 8.09-8.02 (m, 1H), 7.69-7.59 (m, 2H), 7.38-7.30 (m, 1H), 7.22-7.14 (m, 1H), 5.84 (dd, J=23.7, 7.6 Hz, 1H), 5.20 (dt, J=16.5, 7.2 Hz, 1H), 5.11 (t, J=8.9 Hz, 1H), 4.69 (t, J=5.5 Hz, 2H), 4.46 (t, J=5.4 Hz, 2H), 2.51-2.34 (m, 5H), 2.18-2.01 (m, 3H), 1.83-1.68 (m, 3H), 1.55-1.38 (m, 5H). MS (ESI) m/z: [M+H]⁺ Found 640.0.

### Example 36

### N—[(S)-(4,4-Difluorocyclohexyl)-[6-[(1R)-1-(4,4,4-trifluorobutanoylamino)ethyl]-1H-benzimidazol-2-yl]methyl]-3-[2-(trifluoromethoxy)ethyl]triazole-4-carboxamide

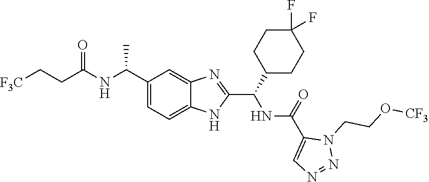 [SMILES: C[C@@H](NC(=O)CCC(F)(F)F)c1ccc2[nH]c([C@@H](NC(=O)c3cnnn3CCOC(F)(F)F)C3CCC(F)(F)CC3)nc2c1]

The title compound was prepared as described for the synthesis of Example 1, using 1-(2-(trifluoromethoxy)ethyl)-1H-1,2,3-triazole-5-carboxylic acid (Intermediate 52) in place of 1-(3,3,3-trifluoropropyl)-1H-pyrazole-4-carboxylic acid, and stirring at rt for 2 h followed by 40° C. for 20 h instead of stirring at rt for 3 h. The title compound was purified by basic preparative HPLC (NH₄OH). ¹H NMR (500 MHz, CDCl₃) δ 8.28-8.24 (m, 1H), 7.63-7.57 (m, 1H), 7.42-7.38 (m, 1H), 7.31 (s, 1H), 7.26-7.20 (m, 1H), 6.99-6.92 (m, 1H), 5.20-5.00 (m, 4H), 4.41-4.37 (m, 2H), 3.40-3.37 (m, 1H), 2.49-2.38 (m, 4H), 2.22-2.12 (m, 2H), 2.08-1.98 (m, 2H), 1.83-1.62 (m, 2H), 1.54-1.43 (m, 5H), 1.39-1.29 (m, 1H). MS (ESI) m/z: [M+H]⁺ Found 640.3.

### Example 37

### N—[(S)-(4,4-Difluorocyclohexyl)-[6-[(1R)-1-(4,4,4-trifluorobutanoylamino)ethyl]-1H-benzimidazol-2-yl]methyl]-2-(2,2-difluoroethyl)triazole-4-carboxamide

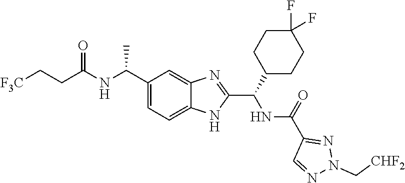 [SMILES: C[C@@H](NC(=O)CCC(F)(F)F)c1ccc2[nH]c([C@@H](NC(=O)c3cnn(CC(F)F)n3)C3CCC(F)(F)CC3)nc2c1]

The title compound was prepared as described for the synthesis of Example 1, using 2-(2,2-difluoroethyl)-2H-1,2,3-triazole-4-carboxylic acid (Intermediate 54) in place of 1-(3,3,3-trifluoropropyl)-1H-pyrazole-4-carboxylic acid, and stirring at rt for 2 h followed by 40° C. for 18 h instead of stirring at rt for 3 h. The title compound was purified by basic preparative HPLC (NH₄OH). ¹H NMR (500 MHz, CDCl₃) δ 10.72 (d, J=67.8 Hz, 1H), 8.08-8.01 (m, 1H), 7.76-7.67 (m, 1H), 7.67-7.61 (m, 1H), 7.34-7.27 (m, 1H), 7.19 (ddd, J=12.8, 8.4, 1.7 Hz, 1H), 6.32-6.05 (m, 1H), 5.87 (dd, J=40.7, 7.6 Hz, 1H), 5.23-5.08 (m, 2H), 4.77-4.68 (m, 2H), 2.50-2.34 (m, 5H), 2.18-2.02 (m, 3H), 1.81-1.66 (m, 3H), 1.52 (dd, J=17.5, 6.9 Hz, 4H), 1.47-1.38 (m, 1H). MS (ESI) m/z: [M+H]⁺ Found 592.2.

### Example 38

### N—[(S)-(4,4-Difluorocyclohexyl)-[6-[(1R)-1-(4,4,4-trifluorobutanoylamino)ethyl]-1H-benzimidazol-2-yl]methyl]-3-(2,2-difluoroethyl)triazole-4-carboxamide

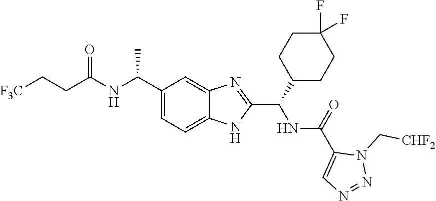 [SMILES: C[C@@H](NC(=O)CCC(F)(F)F)c1ccc2[nH]c([C@@H](NC(=O)c3cnnn3CC(F)F)C3CCC(F)(F)CC3)nc2c1]

The title compound was prepared as described for the synthesis of Example 1, using 1-(2,2-difluoroethyl)-1H-1,2,3-triazole-5-carboxylic acid (Intermediate 56) in place of 1-(3,3,3-trifluoropropyl)-1H-pyrazole-4-carboxylic acid, and stirring at rt for 2 h followed by 40° C. for 18 h instead of stirring at rt for 3 h. The title compound was purified by basic preparative HPLC (NH₄OH). ¹H NMR (500 MHz, CDCl₃) δ 8.31-8.25 (m, 1H), 7.64-7.57 (m, 1H), 7.44-7.39 (m, 1H), 7.26-7.20 (m, 1H), 6.67-6.55 (m, 1H), 6.33-6.03 (m, 1H), 5.22-5.10 (m, 3H), 5.04 (dd, J=9.6, 7.2 Hz, 1H), 3.41 (s, 2H), 2.52-2.36 (m, 5H), 2.16-1.99 (m, 3H), 1.83-1.61 (m, 2H), 1.55-1.32 (m, 6H). MS (ESI) m/z: [M+H]⁺ Found 592.2.

### Example 39

### N—[(S)-(4,4-Difluorocyclohexyl)-[6-[(1R)-1-(4,4,4-trifluorobutanoylamino)ethyl]-1H-benzimidazol-2-yl]methyl]-1-isopropyl-1,2,4-triazole-3-carboxamide

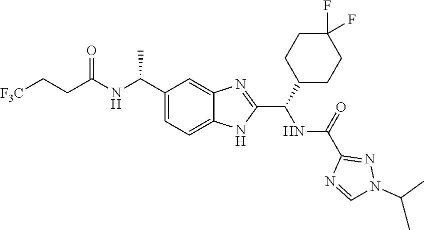 [SMILES: CC(C)n1cnc(C(=O)N[C@H](c2nc3cc([C@@H](C)NC(=O)CCC(F)(F)F)ccc3[nH]2)C2CCC(F)(F)CC2)n1]

The title compound was prepared as described for the synthesis of Example 1, using 1-isopropyl-1H-1,2,4-triazole-3-carboxylic acid in place of 1-(3,3,3-trifluoropropyl)-1H-pyrazole-4-carboxylic acid, and stirring at rt for 2 h followed by 40° C. for 18 h instead of stirring at rt for 3 h.

The title compound was purified by basic preparative HPLC (NH₄OH). ¹H NMR (500 MHz, CDCl₃) δ 11.60-11.23 (m, 1H), 8.17 (d, J=8.1 Hz, 1H), 7.94 (dd, J=12.5, 9.0 Hz, 1H), 7.71-7.61 (m, 1H), 7.58-7.43 (m, 1H), 7.22-7.14 (m, 1H), 5.88 (dd, J=22.6, 7.8 Hz, 1H), 5.48-5.37 (m, 1H), 5.28-5.14 (m, 1H), 4.63-4.54 (m, 1H), 2.54-2.33 (m, 5H), 2.23-2.04 (m, 3H), 1.86-1.69 (m, 3H), 1.64-1.59 (m, 1H), 1.57 (dd, J=6.7, 4.4 Hz, 6H), 1.55-1.51 (m, 3H), 1.51-1.44 (m, 1H). MS (ESI) m/z: [M+H]⁺ Found 570.2.

### Example 40

N—[(S)-(4,4-Difluorocyclohexyl)-[6-[(1R)-1-(4,4,4-trifluorobutanoylamino)ethyl]-1H-benzimidazol-2-yl]methyl]-1-(3,3-difluoropropyl)-1,2,4-triazole-3-carboxamide

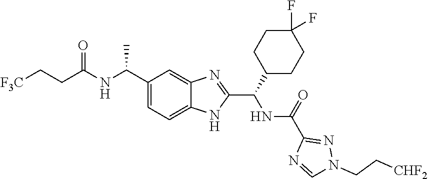 [SMILES: C[C@@H](NC(=O)CCC(F)(F)F)c1ccc2[nH]c([C@@H](NC(=O)c3ncn(CCC(F)F)n3)C3CCC(F)(F)CC3)nc2c1]

The title compound was prepared as described for the synthesis of Example 1, using 1-(3,3-difluoropropyl)-1H-1,2,4-triazole-3-carboxylic acid (Intermediate 58) in place of 1-(3,3,3-trifluoropropyl)-1H-pyrazole-4-carboxylic acid, and stirring at rt for 3 h followed by 40° C. for 17 h instead of just stirring at rt for 3 h. The title compound was purified by basic preparative HPLC (NH₄OH). ¹H NMR (500 MHz, CDCl₃, benzimidazole NH absent from exchange) δ 8.20-8.15 (m, 1H), 7.67-7.59 (m, 1H), 7.39 (s, 1H), 7.24-7.17 (m, 1H), 6.06-5.80 (m, 1H), 5.22-5.10 (m, 2H), 4.46-4.40 (m, 2H), 3.44-3.34 (m, 2H), 2.63 (s, 1H), 2.51-2.42 (m, 5H), 2.37-2.30 (m, 1H), 2.14 (br s, 1H), 2.08-2.02 (m, 2H), 1.83-1.67 (m, 2H), 1.64-1.57 (m, 1H), 1.55-1.48 (m, 4H), 1.42-1.36 (m, 1H). MS (ESI) m/z: [M+H]⁺ Found 606.2.

### Example 41

### N—[(S)-(4,4-Difluorocyclohexyl)-[6-[(1R)-1-(4,4,4-trifluorobutanoylamino)ethyl]-1H-benzimidazol-2-yl]methyl]-2-(3,3-difluoropropyl)-1,2,4-triazole-3-carboxamide

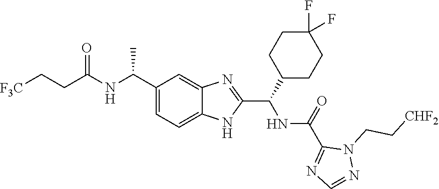 [SMILES: C[C@@H](NC(=O)CCC(F)(F)F)c1ccc2[nH]c([C@@H](NC(=O)c3ncnn3CCC(F)F)C3CCC(F)(F)CC3)nc2c1]

A mixture of N—((R)-1-(2-((S)-amino(4,4-difluorocyclohexyl)methyl)-1H-benzo[d]imidazol-5-yl)ethyl)-4,4,4-trifluorobutanamide (75 mg, 0.17 mmol, Intermediate 4), methyl 1-(3,3-difluoropropyl)-1H-1,2,4-triazole-5-carboxylate (107 mg, 0.52 mmol, Intermediate 59) and 2,2,2-trifluoroethanol (0.87 mL) was stirred at reflux for 6 h. After that time, DMA (0.9 mL) was added and the mixture stirred at 140° C. for 16 h. The reaction was cooled to rt and concentrated to dryness. The residue was purified twice by basic preparative HPLC (NH₄OH) to provide the title compound as a white solid. ¹H NMR (400 MHz, CDCl₃) δ 11.66-11.48 (m, 1H), 8.57-8.42 (m, 1H), 7.90-7.82 (m, 1H), 7.65-7.55 (m, 1H), 7.38-7.28 (m, 1H), 7.21-7.12 (m, 1H), 6.74-6.48 (m, 1H), 5.92 (tt, J=55.9, 4.2 Hz, 1H), 5.20-5.06 (m, 2H), 4.81 (td, J=6.9, 1.9 Hz, 2H), 2.49-2.32 (m, 7H), 2.29-2.21 (m, 1H), 2.04-1.93 (m, 2H), 1.79-1.56 (m, 3H), 1.50-1.34 (m, 5H). MS (ESI) m/z: [M+H]⁺ Found 606.2.

### Example 42

### N—((S)-(5-((S*)-Cyclobutyl(4,4,4-trifluorobutanamido)methyl)-1H-benzo[d]imidazol-2-yl)(4,4-difluorocyclohexyl)methyl)-1-methyl-1H-pyrazole-5-carboxamide

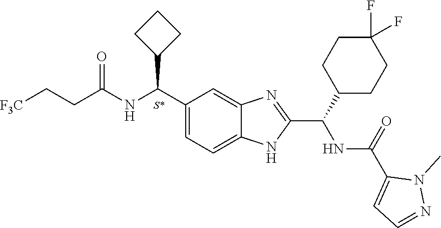 [SMILES: Cn1nccc1C(=O)N[C@H](c1nc2cc([C@@H](NC(=O)CCC(F)(F)F)C3CCC3)ccc2[nH]1)C1CCC(F)(F)CC1]

### Example 43

### N—((S)-(5-((R*)-Cyclobutyl(4,4,4-trifluorobutanamido)methyl)-1H-benzo[d]imidazol-2-yl)(4,4-difluorocyclohexyl)methyl)-1-methyl-1H-pyrazole-5-carboxamide

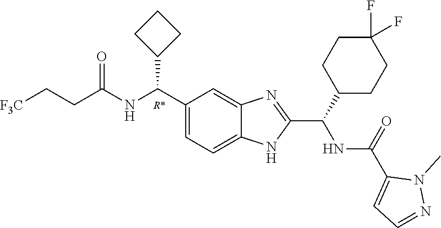 [SMILES: Cn1nccc1C(=O)N[C@H](c1nc2cc([C@H](NC(=O)CCC(F)(F)F)C3CCC3)ccc2[nH]1)C1CCC(F)(F)CC1]

EDCI (180 mg, 0.94 mmol) was added to a solution of 4,4,4-trifluorobutanoic acid (135 mg, 0.995 mmol) and HOAt (144 mg, 1.06 mmol) in DCM (5 mL) and the resulting mixture was stirred at rt for 10 min. Then, N-((1S)-(5-(amino(cyclobutyl)methyl)-1H-benzo[d]imidazol-2-yl)(4,4-difluorocyclohexyl)methyl)-1-methyl-1H-pyrazole-5-carboxamide (360 mg, 0.79 mmol, Intermediate 65) and DIPEA (0.29 mL, 1.66 mmol) were added and the mixture was stirred for 2 h at rt. At that point, the mixture was partitioned between water (5 mL) and DCM (10 mL). The layers were separated and the aqueous further extracted with DCM (2×10 mL). The organic layers were combined and concentrated to dryness. The residue was purified by preparative basic HPLC (Xtimate 10 μm, C18, 250×50 mm, 35-65% acetonitrile/water (with 0.04% NH₄OH and 10 mM NH₄HCO₃). The product containing fractions were diluted with water, frozen, and lyophilized to afford the title compound, a mixture of diastereomers, as a white solid. The diastereomers were separated by SFC using a chiral stationary phase (Phenomenex-Amylose-1, 5 μm, 250×30 mm, mobile phase: 25% CO₂ in EtOH (0.1% NH₄OH)). The first eluting isomer was Example 43 and the second eluting isomer was Example 42. Example 42: ¹H NMR (400 MHz, DMSO-d₆) δ 12.36 (br s, 1H), 8.94-8.84 (m, 1H), 8.43-8.32 (m, 1H), 7.54-7.42 (m, 2H), 7.40-7.32 (m, 1H), 7.12-7.03 (m, 2H), 5.17-5.09 (m, 1H), 4.88-4.79 (m, 1H), 4.02 (s, 3H), 2.70-2.58 (m, 1H), 2.47-2.21 (m, 5H), 2.10-1.91 (m, 4H), 1.85-1.64 (m, 7H), 1.59-1.50 (m, 1H), 1.45-1.24 (m, 2H). MS (ESI) m/z: [M+H]⁺ Found 581.3. Example 43: ¹H NMR (400 MHz, DMSO-d₆) δ 12.37 (br s, 1H), 8.94-8.86 (m, 1H), 8.43-8.33 (m, 1H), 7.52-7.44 (m, 2H), 7.41-7.31 (m, 1H), 7.12-7.02 (m, 2H), 5.18-5.08 (m, 1H), 4.89-4.79 (m, 1H), 4.02 (s, 3H), 2.70-2.58 (m, 1H), 2.47-2.22 (m, 5H), 2.09-1.93 (m, 4H), 1.86-1.64 (m, 7H), 1.60-1.49 (m, 1H), 1.44-1.25 (m, 2H). MS (ESI) m/z: [M+H]⁺ Found 581.3.

### Example 44

### N—((S)-(4,4-Difluorocyclohexyl)(5-((S*)-2-methyl-1-(4,4,4-trifluorobutanamido)propyl)-1H-benzo[d]imidazol-2-yl)methyl)-1-methyl-1H-pyrazole-5-carboxamide

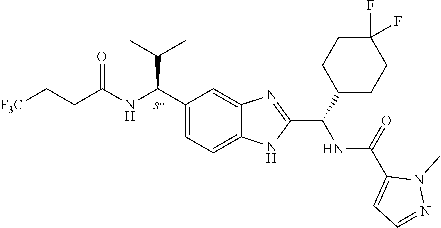 [SMILES: CC(C)[C@H](NC(=O)CCC(F)(F)F)c1ccc2[nH]c([C@@H](NC(=O)c3ccnn3C)C3CCC(F)(F)CC3)nc2c1]

### Example 45

### N—((S)-(4,4-Difluorocyclohexyl)(5-((R*)-2-methyl-1-(4,4,4-trifluorobutanamido)propyl)-1H-benzo[d]imidazol-2-yl)methyl)-1-methyl-1H-pyrazole-5-carboxamide

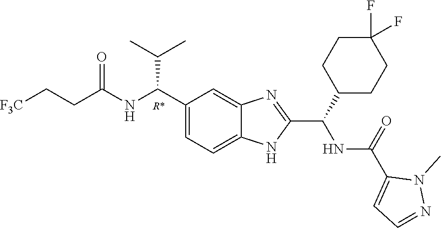 [SMILES: CC(C)[C@@H](NC(=O)CCC(F)(F)F)c1ccc2[nH]c([C@@H](NC(=O)c3ccnn3C)C3CCC(F)(F)CC3)nc2c1]

The title compounds were prepared as described for the synthesis of Example CLM1, using N-((1S)-(5-(1-amino-2-methylpropyl)-1H-benzo[d]imidazol-2-yl)(4,4-difluorocyclohexyl)methyl)-1-methyl-1H-pyrazole-5-carboxamide (Intermediate 66) in place of N-((1S)-(5-(amino(cyclobutyl)methyl)-1H-benzo[d]imidazol-2-yl)(4,4-difluorocyclohexyl)methyl)-1-methyl-1H-pyrazole-5-carboxamide and purified by preparative basic HPLC (Boston Prime 5 μm, C18, 150×30 mm, 40-70% acetonitrile/water (with 0.04% NH₄OH and 10 mM NH₄HCO₃). The product containing fractions were diluted with water, frozen, and lyophilized to afford the title compound, a mixture of diastereomers, as a white solid. The diastereomers were separated by SFC using a chiral stationary phase (REGIS (s,s) WHELK-O1, 5 μm, 250×30 mm, mobile phase: 30% CO₂ in EtOH (0.1% NH₄OH)). The first eluting isomer was Example 45 and the second eluting isomer was Example 44. Example 44: ¹H NMR (400 MHz, DMSO-d₆) δ 12.36 (br s, 1H), 8.96-8.82 (m, 1H), 8.47-8.31 (m, 1H), 7.54-7.48 (m, 1H), 7.47 (d, J=1.7 Hz, 1H), 7.40-7.30 (m, 1H), 7.12-7.04 (m, 2H), 5.19-5.08 (m, 1H), 4.67-4.53 (m, 1H), 4.03 (s, 3H), 2.48-2.26 (m, 5H), 2.14-1.90 (m, 4H), 1.89-1.68 (m, 2H), 1.61-1.51 (m, 1H), 1.44-1.23 (m, 2H), 0.96-0.87 (m, 3H), 0.70 (d, J=6.1 Hz, 3H). MS (ESI) m/z: [M+H]⁺ Found 569.3. Example 45: ¹H NMR (400 MHz, DMSO-d₆) δ 12.36 (br s, 1H), 9.00-8.83 (m, 1H), 8.47-8.33 (m, 1H), 7.56-7.43 (m, 2H), 7.41-7.32 (m, 1H), 7.14-7.02 (m, 2H), 5.20-5.09 (m, 1H), 4.66-4.57 (m, 1H), 4.03 (s, 3H), 2.47-2.21 (m, 5H), 2.13-1.91 (m, 4H), 1.90-1.67 (m, 2H), 1.62-1.49 (m, 1H), 1.47-1.18 (m, 2H), 0.91 (d, J=6.4 Hz, 3H), 0.70 (d, J=6.4 Hz, 3H). MS (ESI) m/z: [M+H]⁺ Found 569.3.

### Example 46

### N—((S)-(5-((S*)-2-Cyclobutyl-1-(4,4,4-trifluorobutanamido)ethyl)-1H-benzo[d]imidazol-2-yl)(4,4-difluorocyclohexyl)methyl)-1-methyl-1H-pyrazole-5-carboxamide

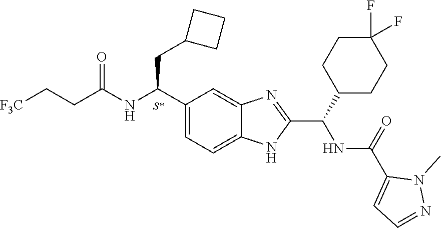 [SMILES: Cn1nccc1C(=O)N[C@H](c1nc2cc([C@H](CC3CCC3)NC(=O)CCC(F)(F)F)ccc2[nH]1)C1CCC(F)(F)CC1]

### Example 47

### N—((S)-(5-((R*)-2-Cyclobutyl-1-(4,4,4-trifluorobutanamido)ethyl)-1H-benzo[d]imidazol-2-yl)(4,4-difluorocyclohexyl)methyl)-1-methyl-1H-pyrazole-5-carboxamide

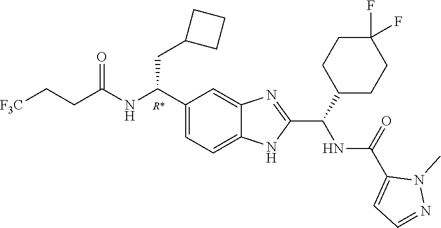 [SMILES: Cn1nccc1C(=O)N[C@H](c1nc2cc([C@@H](CC3CCC3)NC(=O)CCC(F)(F)F)ccc2[nH]1)C1CCC(F)(F)CC1]

The title compounds were prepared as described for the synthesis of Example 42, using N-((1S)-(5-(1-amino-2-cyclobutylethyl)-1H-benzo[d]imidazol-2-yl)(4,4-difluorocyclohexyl)methyl)-1-methyl-1H-pyrazole-5-carboxamide (Intermediate 67) in place of N-((1S)-(5-(amino(cyclobutyl)methyl)-1H-benzo[d]imidazol-2-yl)(4,4-difluorocyclohexyl)methyl)-1-methyl-1H-pyrazole-5-carboxamide. After 16 h at rt, additional aliquots of EDCI (60 mg, 0.31 mmol), HOAt (48 mg, 0.35 mmol), DIPEA (0.09 mL, 0.52 mmol) and 4,4,4-trifluorobutanoic acid (42 mg, 0.30 mmol) were added and the mixture stirred for a further 2 h at rt. The crude material was purified by preparative basic HPLC (Xtimate 10 μm, C18, 250×50 mm, 45-75% acetonitrile/water (with 0.04% NH₄OH and 10 mM NH₄HCO₃). The product containing fractions were diluted with water, frozen and lyophilized to afford the title compound, a mixture of diastereomers, as a white solid. The diastereomers were separated by SFC using a chiral stationary phase (REGIS (s,s) WHELK-O1, 5 μm, 250×30 mm, mobile phase: 40% CO₂ in EtOH (0.1% NH₃)). The first eluting isomer was Example 47 and the second eluting isomer was Example 46. Example 46: ¹H NMR (400 MHz, DMSO-d₆) δ 12.40-12.32 (m, 1H), 8.95-8.87 (m, 1H), 8.45-8.36 (m, 1H), 7.53-7.44 (m, 2H), 7.40-7.31 (m, 1H), 7.11-7.04 (m, 2H), 5.16-5.10 (m, 1H), 4.81-4.72 (m, 1H), 4.02 (s, 3H), 2.47-2.32 (m, 4H), 2.31-2.23 (m, 1H), 2.22-2.12 (m, 1H), 2.11-1.92 (m, 4H), 1.88-1.70 (m, 7H), 1.70-1.61 (m, 1H), 1.60-1.50 (m, 2H), 1.39-1.23 (m, 2H). MS (ESI) m/z: [M+H]⁺ Found 595.3. Example 47: ¹H NMR (400 MHz, DMSO-d₆) δ 12.42 (br s, 1H), 8.99-8.88 (m, 1H), 8.50-8.35 (m, 1H), 7.53-7.43 (m, 2H), 7.41-7.31 (m, 1H), 7.12-7.04 (m, 2H), 5.18-5.10 (m, 1H), 4.81-4.72 (m, 1H), 4.03 (s, 3H), 2.46-2.30 (m, 4H), 2.29-2.22 (m, 1H), 2.22-2.12 (m, 1H), 2.11-1.91 (m, 4H), 1.91-1.70 (m, 7H), 1.70-1.62 (m, 1H), 1.61-1.49 (m, 2H), 1.43-1.22 (m, 2H). MS (ESI) m/z: [M+H]⁺ Found 595.3.

### Example 48

### N—((S)-(4,4-Difluorocyclohexyl)(5-((S*)-3-methyl-1-(4,4,4-trifluorobutanamido)butyl)-1H-benzo[d]imidazol-2-yl)methyl)-1-methyl-1H-pyrazole-5-carboxamide

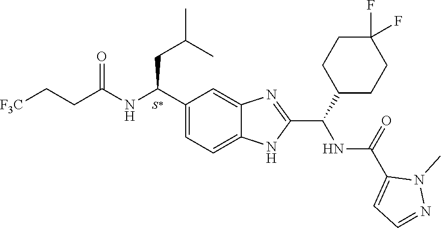 [SMILES: CC(C)C[C@H](NC(=O)CCC(F)(F)F)c1ccc2[nH]c([C@@H](NC(=O)c3ccnn3C)C3CCC(F)(F)CC3)nc2c1]

### Example 49

### N—((S)-(4,4-Difluorocyclohexyl)(5-((R*)-3-methyl-1-(4,4,4-trifluorobutanamido)butyl)-1H-benzo[d]imidazol-2-yl)methyl)-1-methyl-1H-pyrazole-5-carboxamide

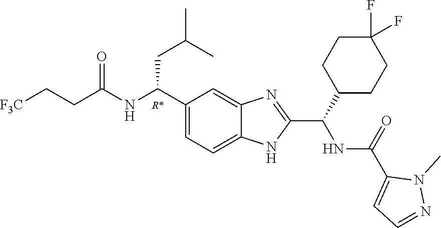 [SMILES: CC(C)C[C@@H](NC(=O)CCC(F)(F)F)c1ccc2[nH]c([C@@H](NC(=O)c3ccnn3C)C3CCC(F)(F)CC3)nc2c1]

EDCI (150 mg, 0.78 mmol) was added to a solution of 4,4,4-trifluorobutanoic acid (110 mg, 0.77 mmol) and HOAt (120 mg, 0.88 mmol) in DCM (10 mL) and the resulting mixture was stirred at rt for 10 min. Then, N-((1S)-(5-(1-amino-3-methylbutyl)-1H-benzo[d]imidazol-2-yl)(4,4-difluorocyclohexyl)methyl)-1-methyl-1H-pyrazole-5-carboxamide (1.53 g crude, Intermediate 68) and DIPEA (0.25 mL, 1.4 mmol) were added and the mixture was stirred for 16 h at rt. Then, an additional aliquot of EDCI (75 mg, 0.39 mmol), HOAt (60 mg, 0.44 mmol), DIPEA (0.12 mL, 0.7 mmol) and 4,4,4-trifluorobutanoic acid (55 mg, 1.2 mmol) were added and the mixture stirred for a further 3 h at rt. The reaction mixture was then concentrated to dryness and then partitioned between water (30 mL) and EtOAc (40 mL). The layers were separated and the aqueous further extracted with EtOAc (2×40 mL). Then the organic layers were combined, filtered through a pad of Celite®, rinsing with EtOAc (60 mL) and concentrated to dryness. The crude material was purified by preparative basic HPLC (Boston Prime 5 μm, C18, 150×30 mm, 50-80% acetonitrile/water (with 0.04% NH₄OH and 10 mM NH₄HCO₃). The product containing fractions were diluted with water, frozen, and lyophilized to afford the title compound, a mixture of diastereomers, as a white solid. The diastereomers were separated by SFC using a chiral stationary phase (Phenomenex-Cellulose-2, 5 μm, 250×30 mm, mobile phase: 30% CO₂ in EtOH/water (0.1% NH₃)). The first eluting isomer was Example 49 and the second eluting isomer was Example 48. Example 48: ¹H NMR (400 MHz, DMSO-d₆) δ 12.43-12.27 (m, 1H), 8.92 (d, J=8.3 Hz, 1H), 8.48-8.39 (m, 1H), 7.53-7.43 (m, 2H), 7.42-7.32 (m, 1H), 7.13-7.03 (m, 2H), 5.17-5.09 (m, 1H), 4.99-4.89 (m, 1H), 4.02 (s, 3H), 2.45-2.25 (m, 4H), 2.12-1.91 (m, 3H), 1.90-1.70 (m, 2H), 1.70-1.59 (m, 1H), 1.59-1.45 (m, 3H), 1.44-1.17 (m, 3H), 0.92-0.83 (m, 6H). MS (ESI) m/z: [M+H]⁺ Found 583.3. Example 49: ¹H NMR (400 MHz, DMSO-d₆) δ 12.36 (br s, 1H), 9.00-8.83 (m, 1H), 8.51-8.36 (m, 1H), 7.55-7.43 (m, 2H), 7.42-7.32 (m, 1H), 7.15-7.02 (m, 2H), 5.17-5.09 (m, 1H), 4.99-4.88 (m, 1H), 4.02 (s, 3H), 2.45-2.22 (m, 4H), 2.13-1.90 (m, 3H), 1.90-1.71 (m, 2H), 1.70-1.59 (m, 1H), 1.59-1.45 (m, 3H), 1.44-1.19 (m, 3H), 0.94-0.82 (m, 6H). MS (ESI) m/z: [M+H]⁺ Found 583.3.

### Example 50

### N—((S)-(4,4-Difluorocyclohexyl)(5-((S*)-1-(4,4,4-trifluorobutanamido)butyl)-1H-benzo[d]imidazol-2-yl)methyl)-1-methyl-1H-pyrazole-5-carboxamide

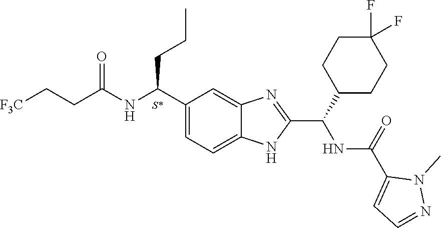 [SMILES: CCC[C@H](NC(=O)CCC(F)(F)F)c1ccc2[nH]c([C@@H](NC(=O)c3ccnn3C)C3CCC(F)(F)CC3)nc2c1]

### Example 51

### N—((S)-(4,4-Difluorocyclohexyl)(5-((R*)-1-(4,4,4-trifluorobutanamido)butyl)-1H-benzo[d]imidazol-2-yl)methyl)-1-methyl-1H-pyrazole-5-carboxamide

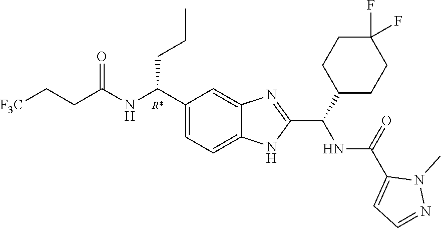 [SMILES: CCC[C@@H](NC(=O)CCC(F)(F)F)c1ccc2[nH]c([C@@H](NC(=O)c3ccnn3C)C3CCC(F)(F)CC3)nc2c1]

EDCI (620 mg, 3.23 mmol) and HOAt (450 mg, 3.31 mmol) were added to a solution of N—((1S)-(5-(1-aminobutyl)-1H-benzo[d]imidazol-2-yl)(4,4-difluorocyclohexyl)methyl)-1-methyl-1H-pyrazole-5-carboxamide hydrochloride (1.3 g, 2.7 mmol, Intermediate 69), 4,4,4-trifluorobutanoic acid (460 mg, 3.24 mmol) and DIPEA (3 mL, 18.2 mmol) in DCM (10 mL) and the resulting mixture was stirred at rt overnight. The mixture was then concentrated to dryness and purified by silica gel chromatography (0-100% EtOAc/petroleum ether) to afford the title compound, a mixture of diastereomers, as a yellow solid. The diastereomers were separated by SFC using a chiral stationary phase (Phenomenex-Cellulose-2, 5 μm, 250×30 mm, mobile phase: 35% CO₂ in EtOH/water (0.1% NH₄OH)). The first eluting isomer was Example 51 and the second eluting isomer was Example 50. Example 50: ¹H NMR (400 MHz, DMSO-d₆) δ 12.41 (br s, 1H), 8.98-8.90 (m, 1H), 8.49-8.38 (m, 1H), 7.54-7.45 (m, 2H), 7.41-7.33 (m, 1H), 7.15-7.05 (m, 2H), 5.18-5.10 (m, 1H), 4.90-4.81 (m, 1H), 4.03 (s, 3H), 2.49-2.22 (m, 5H), 2.13-1.91 (m, 3H), 1.90-1.50 (m, 5H), 1.45-1.13 (m, 4H), 0.86 (t, J=7.3 Hz, 3H). MS (ESI) m/z: [M+H]⁺ Found 569.2. Example 51: ¹H NMR (400 MHz, DMSO-d₆) δ 12.37 (s, 1H), 8.97-8.88 (m, 1H), 8.46-8.38 (m, 1H), 7.53-7.44 (m, 2H), 7.41-7.33 (m, 1H), 7.15-7.04 (m, 2H), 5.18-5.09 (m, 1H), 4.92-4.81 (m, 1H), 4.03 (s, 3H), 2.48-2.22 (m, 5H), 2.13-1.91 (m, 3H), 1.87-1.51 (m, 5H), 1.45-1.12 (m, 4H), 0.86 (t, J=7.2 Hz, 3H). MS (ESI) m/z: [M+H]⁺ Found 569.1.

### Example 52

### N—((S)-(4,4-Difluorocyclohexyl)(5-((S*)-1-(4,4,4-trifluorobutanamido)propyl)-1H-benzo[d]imidazol-2-yl)methyl)-1-methyl-1H-pyrazole-5-carboxamide

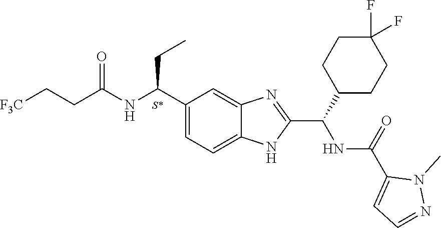 [SMILES: CC[C@H](NC(=O)CCC(F)(F)F)c1ccc2[nH]c([C@@H](NC(=O)c3ccnn3C)C3CCC(F)(F)CC3)nc2c1]

### Example 53

### N—((S)-(4,4-Difluorocyclohexyl)(5-((R*)-1-(4,4,4-trifluorobutanamido)propyl)-1H-benzo[d]imidazol-2-yl)methyl)-1-methyl-1H-pyrazole-5-carboxamide

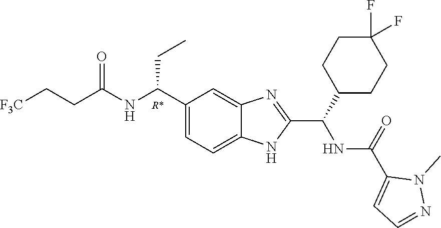 [SMILES: CC[C@@H](NC(=O)CCC(F)(F)F)c1ccc2[nH]c([C@@H](NC(=O)c3ccnn3C)C3CCC(F)(F)CC3)nc2c1]

The title compounds were prepared as described for the synthesis of Example 50, using N—((1S)-(5-(1-aminopropyl)-1H-benzo[d]imidazol-2-yl)(4,4-difluorocyclohexyl)methyl)-1-methyl-1H-pyrazole-5-carboxamide hydrochloride (Intermediate 70) in place of N-((1S)-(5-(1-aminobutyl)-1H-benzo[d]imidazol-2-yl)(4,4-difluorocyclohexyl)methyl)-1-methyl-1H-pyrazole-5-carboxamide hydrochloride to afford the title compound, a mixture of diastereomers, as a white solid. The diastereomers were separated by SFC using a chiral stationary phase (REGIS (s,s) WHELK-01, 5 μm, 250×30 mm, mobile phase: 45% CO₂ in EtOH (0.1% NH₄OH)). The first eluting isomer was Example 53 and the second eluting isomer was Example 52. Example 52: ¹H NMR (400 MHz, DMSO-d₆) δ 12.40 (br s, 1H), 8.93 (d, J=8.3 Hz, 1H), 8.49-8.38 (m, 1H), 7.55-7.32 (m, 3H), 7.14-7.02 (m, 2H), 5.19-5.08 (m, 1H), 4.82-4.71 (m, 1H), 4.02 (s, 3H), 2.47-2.32 (m, 4H), 2.12-1.91 (m, 3H), 1.89-1.67 (m, 4H), 1.59-1.48 (m, 1H), 1.45-1.19 (m, 3H), 0.88-0.78 (m, 3H). MS (ESI) m/z: [M+H]⁺ Found 555.3. Example 53: ¹H NMR (400 MHz, DMSO-d₆) δ 12.51 (br s, 1H), 9.08-8.83 (m, 1H), 8.62-8.28 (m, 1H), 7.53-7.31 (m, 3H), 7.16-7.05 (m, 2H), 5.24-5.06 (m, 1H), 4.86-4.67 (m, 1H), 4.02 (s, 3H), 2.48-2.32 (m, 4H), 2.13-1.90 (m, 3H), 1.87-1.68 (m, 4H), 1.60-1.49 (m, 1H), 1.46-1.19 (m, 3H), 0.87-0.78 (m, 3H). MS (ESI) m/z: [M+H]⁺ Found 555.3.

### Example 54

### N—((S)-(5-((S*)-Cyclopropyl(4,4,4-trifluorobutanamido)methyl)-1H-benzo[d]imidazol-2-yl)(4,4-difluorocyclohexyl)methyl)-1-methyl-1H-pyrazole-5-carboxamide

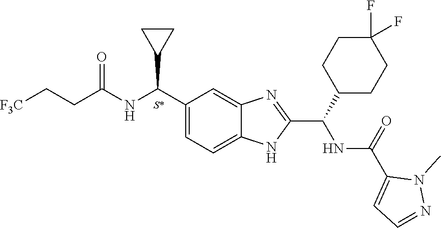 [SMILES: Cn1nccc1C(=O)N[C@H](c1nc2cc([C@@H](NC(=O)CCC(F)(F)F)C3CC3)ccc2[nH]1)C1CCC(F)(F)CC1]

### Example 55

### N—((S)-(5-((R*)-Cyclopropyl(4,4,4-trifluorobutanamido)methyl)-1H-benzo[d]imidazol-2-yl)(4,4-difluorocyclohexyl)methyl)-1-methyl-1H-pyrazole-5-carboxamide

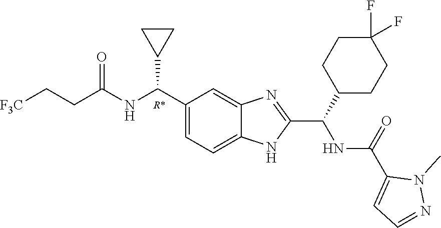 [SMILES: Cn1nccc1C(=O)N[C@H](c1nc2cc([C@H](NC(=O)CCC(F)(F)F)C3CC3)ccc2[nH]1)C1CCC(F)(F)CC1]

EDCI (500 mg, 2.61 mmol) was added to a solution of 4,4,4-trifluorobutanoic acid (366 mg, 2.58 mmol) and HOAt (375 mg, 2.76 mmol) in DCM (4 mL) and the resulting mixture was stirred at rt for 20 min. Then, N-((1S)-(5-(amino(cyclopropyl)methyl)-1H-benzo[d]imidazol-2-yl)(4,4-difluorocyclohexyl)methyl)-1-methyl-1H-pyrazole-5-carboxamide (1.8 g, 4.07 mmol, Intermediate 71) and DIPEA (3 mL, 18.2 mmol) were added and the mixture stirred at rt for 16 h. After that time, additional aliquots of 4,4,4-trifluorobutanoic acid (366 mg, 2.58 mmol), EDCI (500 mg, 2.61 mmol), HOAt (375 mg, 2.76 mmol) and DIPEA (0.45 mL, 2.58 mmol) were added and the mixture was stirred for 16 h at rt. The mixture was then poured into water (10 mL) and extracted with DCM (3×10 mL). The organic extracts were combined, washed with brine, dried over anhydrous Na₂SO₄, filtered, and concentrated to dryness to provide the title compound, a mixture of diastereomers. The crude material was purified by preparative basic HPLC (Phenomenex Gemini 10 μm, C18, 150×25 mm, 35-65% acetonitrile/water (with 0.04% NH₄OH and 10 mM NH₄HCO₃). The product containing fractions were diluted with water, frozen, and lyophilized to afford the title compound, a mixture of diastereomers, as a white solid. The diastereomers were separated by SFC using a chiral stationary phase (DAICEL CHIRALCEL OD-H, 5 μm, 250×30 mm, mobile phase: 45% CO₂ in EtOH (0.1% NH₄OH)). The first eluting isomer was Example 5 and the second eluting isomer was Example 54. Example 54: 1H NMR (400 MHz, DMSO-d₆) δ 12.45-12.32 (m, 1H), 8.92 (dd, J=3.9, 8.6 Hz, 1H), 8.65-8.50 (m, 1H), 7.58-7.48 (m, 1H), 7.47 (d, J=1.7 Hz, 1H), 7.44-7.37 (m, 1H), 7.20-7.12 (m, 1H), 7.10-7.06 (m, 1H), 5.19-5.08 (m, 1H), 4.42-4.32 (m, 1H), 4.02 (s, 3H), 2.46-2.32 (m, 3H), 2.31-2.22 (m, 1H), 2.11-1.91 (m, 3H), 1.88-1.68 (m, 2H), 1.60-1.50 (m, 1H), 1.48-1.11 (m, 4H), 0.55-0.40 (m, 2H), 0.39-0.25 (m, 2H). MS (ESI) m/z: [M+H]⁺ Found 567.3. Example 55: ¹H NMR (400 MHz, DMSO-d₆) δ 12.43-12.33 (m, 1H), 8.91 (dd, J=2.7, 8.6 Hz, 1H), 8.64-8.52 (m, 1H), 7.60-7.49 (m, 1H), 7.47 (d, J=1.7 Hz, 1H), 7.43-7.37 (m, 1H), 7.19-7.12 (m, 1H), 7.09-7.05 (m, 1H), 5.18-5.10 (m, 1H), 4.41-4.32 (m, 1H), 4.02 (s, 3H), 2.46-2.32 (m, 3H), 2.32-2.22 (m, 1H), 2.11-1.92 (m, 3H), 1.87-1.69 (m, 2H), 1.61-1.50 (m, 1H), 1.48-1.10 (m, 4H), 0.55-0.41 (m, 2H), 0.37-0.28 (m, 2H). MS (ESI) m/z: [M+H]⁺ Found 567.3.

### Example 56

### 3-Isopropyl-4-(((S*)-4,4,4-trifluoro-3,3-dimethyl-1-(5-((R)-1-(4,4,4-trifluorobutanamido)ethyl)-1H-benzo[d]imidazol-2-yl)butyl)carbamoyl)-1,2,5-oxadiazole 2-oxide

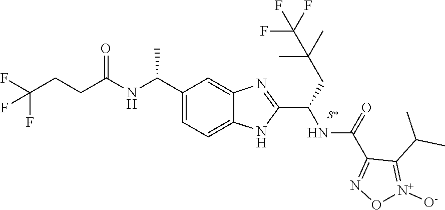 [SMILES: CC(C)c1c(C(=O)N[C@@H](CC(C)(C)C(F)(F)F)c2nc3cc([C@@H](C)NC(=O)CCC(F)(F)F)ccc3[nH]2)no[n+]1[O-]]

DIPEA (0.16 mL, 0.9 mmol) and HATU (230 mg, 0.59 mmol) were sequentially added to a solution of 4-carboxy-3-isopropyl-1,2,5-oxadiazole 2-oxide (102 mg, 0.59 mmol, Intermediate 72) in DMF (2.3 mL). The mixture was allowed to stir at rt for 5 min followed by the addition of N—((R)-1-(2-((S*)-1-amino-4,4,4-trifluoro-3,3-dimethylbutyl)-1H-benzo[d]imidazol-5-yl)ethyl)-4,4,4-trifluorobutanamide (200 mg, 0.46 mmol, Intermediate 281). The reaction was sealed and stirred at rt for 2 h, followed by the addition of extra aliquots of 4-carboxy-3-isopropyl-1,2,5-oxadiazole 2-oxide (39 mg, 0.23 mmol, 72), DIPEA (0.039 mL, 0.23 mmol) and HATU (88 mg, 0.23 mmol). The mixture was stirred at rt for an additional 45 min. The crude reaction mixture was filtered and directly purified by preparative HPLC ((Xbridge Prep C18, 5 mm, 50×100 mm), 10-100% MeCN/aqueous 20 mM NH₄OH) to afford the title compound as an off-white solid. ¹H NMR (500 MHz, DMSO-d₆) δ 12.31 (s, 1H), 9.85 (d, J=8.5 Hz, 1H), 8.44 (d, J=8.0 Hz, 1H), 7.68-7.33 (m, 2H), 7.13 (d, J=8.2 Hz, 1H), 5.45 (td, J=8.6, 4.2 Hz, 1H), 5.02 (quin, J=7.1 Hz, 1H), 3.42 (quin, J=7.0 Hz, 1H), 2.58 (dd, J=14.7, 4.2 Hz, 1H), 2.47-2.23 (m, 5H), 1.38 (d, J=7.0 Hz, 3H), 1.23 (t, J=7.3 Hz, 6H), 1.19 (s, 3H), 1.13 (s, 3H). MS (ESI) m/z: [M+H]⁺ Found 593.2.

### Example 57

### 4-Isopropyl-N—((S*)-4,4,4-trifluoro-3,3-dimethyl-1-(5-((R)-1-(4,4,4-trifluorobutanamido)ethyl)-1H-benzo[d]imidazol-2-yl)butyl)-1,2,5-oxadiazole-3-carboxamide

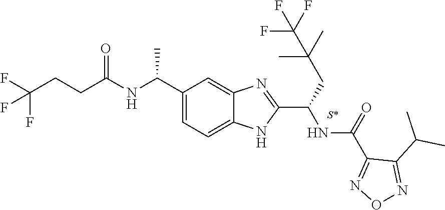 [SMILES: CC(C)c1nonc1C(=O)N[C@@H](CC(C)(C)C(F)(F)F)c1nc2cc([C@@H](C)NC(=O)CCC(F)(F)F)ccc2[nH]1]

The title compound was prepared as described for the synthesis of Example 56, using 4-isopropyl-1,2,5-oxadiazole-3-carboxylic acid (Intermediate 76) in place of 4-carboxy-3-isopropyl-1,2,5-oxadiazole 2-oxide. ¹H NMR (400 MHz, DMSO-d₆) δ 12.31 (s, 1H), 9.84 (dd, J=8.5, 3.5 Hz, 1H), 8.44 (dd, J=13.0, 8.0 Hz, 1H), 7.67-7.45 (m, 1H), 7.45-7.34 (m, 1H), 7.13 (td, J=8.5, 1.6 Hz, 1H), 5.47 (td, J=8.6, 4.0 Hz, 1H), 5.02 (td, J=7.3, 3.4 Hz, 1H), 3.44 (quin, J=6.9 Hz, 1H), 2.58 (dd, J=14.8, 4.0 Hz, 1H), 2.48-2.25 (m, 5H), 1.38 (d, J=6.9 Hz, 3H), 1.31 (dd, J=6.9, 4.5 Hz, 6H), 1.19 (s, 3H), 1.14 (s, 3H). MS (ESI) m/z: [M+H]⁺ Found 577.2.

### Example 58

### N—((S)-(4,4-Difluorocyclohexyl)(6-((R)-1-(4,4,4-trifluorobutanamido)ethyl)-1H-benzo[d]imidazol-2-yl)methyl)-1-methyl-5-(trifluoromethyl)-1H-pyrazole-4-carboxamide

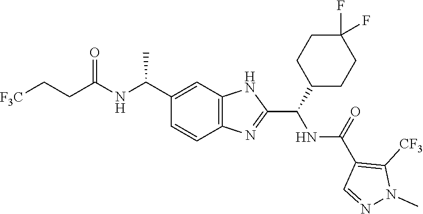 [SMILES: C[C@@H](NC(=O)CCC(F)(F)F)c1ccc2nc([C@@H](NC(=O)c3cnn(C)c3C(F)(F)F)C3CCC(F)(F)CC3)[nH]c2c1]

A vial was charged with 1-methyl-5-(trifluoromethyl)-1H-pyrazole-4-carboxylic acid (40 mg, 0.21 mmol), HATU (132 mg, 0.35 mmol), DIPEA (0.12 mL, 0.7 mmol) and CH₃CN (2 mL). The solution was stirred for 20 min then N—((R)-1-(2-((S)-amino(4,4-difluorocyclohexyl)methyl)-1H-benzo[d]imidazol-6-yl)ethyl)-4,4,4-trifluorobutanamide (75 mg, 0.17 mmol, Intermediate 4) was added and the reaction was stirred for a further 18 h. The crude material was purified directly by silica gel chromatography (0-100% EtOAc (with 10% MeOH)/hexanes) to provide the title compound. ¹H NMR (500 MHz, DMSO-d₆) δ 12.30 (d, J=5.1 Hz, 1H), 8.97-8.85 (m, 1H), 8.48-8.36 (m, 1H), 7.98 (d, J=2.2 Hz, 1H), 7.56-7.46 (m, 1H), 7.45-7.33 (m, 1H), 7.17-7.05 (m, 1H), 5.18-5.10 (m, 1H), 5.08-4.94 (m, 1H), 4.06-3.92 (m, 3H), 2.49-2.32 (m, 4H), 2.26-2.15 (m, 1H), 2.10-1.87 (m, 3H), 1.87-1.66 (m, 2H), 1.58-1.48 (m, 1H), 1.45-1.33 (m, 4H), 1.32-1.19 (m, 1H). MS (ESI) m/z: [M+H]+ Found 609.3.

### Example 59

### N—((S)-(4,4-Difluorocyclohexyl)(6-((R)-1-(4,4,4-trifluorobutanamido)ethyl)-1H-benzo[d]imidazol-2-yl)methyl)-5-(difluoromethyl)-1-methyl-1H-pyrazole-4-carboxamide

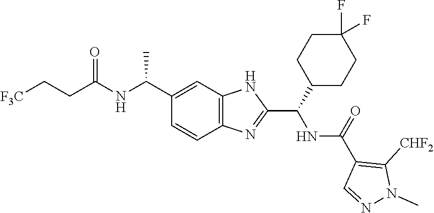 [SMILES: C[C@@H](NC(=O)CCC(F)(F)F)c1ccc2nc([C@@H](NC(=O)c3cnn(C)c3C(F)F)C3CCC(F)(F)CC3)[nH]c2c1]

The title compound was prepared as described for the synthesis of Example 58, using 5-(difluoromethyl)-1-methyl-1H-pyrazole-4-carboxylic acid in place of 1-methyl-5-(trifluoromethyl)-1H-pyrazole-4-carboxylic acid to provide the title compound. ¹H NMR (500 MHz, DMSO-d₆) δ 12.37-12.22 (m, 1H), 8.94-8.83 (m, 1H), 8.49-8.36 (m, 1H), 8.25 (d, J=1.1 Hz, 1H), 7.82-7.53 (m, 1H), 7.53-7.47 (m, 1H), 7.43-7.32 (m, 1H), 7.17-7.02 (m, 1H), 5.17-5.10 (m, 1H), 5.07-4.94 (m, 1H), 3.98 (s, 3H), 2.48-2.32 (m, 4H), 2.32-2.21 (m, 1H), 2.09-1.91 (m, 3H), 1.88-1.67 (m, 2H), 1.61-1.50 (m, 1H), 1.44-1.33 (m, 4H), 1.33-1.19 (m, 1H). MS (ESI) m/z: [M+H]⁺ Found 591.3.

### Example 60

### 1-Cyclopropyl-N—((S)-(4,4-difluorocyclohexyl)(6-((R)-1-(4,4,4-trifluorobutanamido)ethyl)-1H-benzo[d]imidazol-2-yl)methyl)-1H-1,2,3-triazole-5-carboxamide

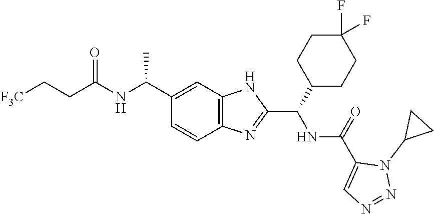 [SMILES: C[C@@H](NC(=O)CCC(F)(F)F)c1ccc2nc([C@@H](NC(=O)c3cnnn3C3CC3)C3CCC(F)(F)CC3)[nH]c2c1]

The title compound was prepared as described for the synthesis of Example 58, using 1-cyclopropyl-1H-1,2,3-triazole-5-carboxylic acid in place of 1-methyl-5-(trifluoromethyl)-1H-pyrazole-4-carboxylic acid to provide the title compound. ¹H NMR (400 MHz, DMSO-d₆) δ 12.38 (s, 1H), 9.25 (d, J=8.5 Hz, 1H), 8.49-8.37 (m, 1H), 8.31 (s, 1H), 7.56-7.47 (m, 1H), 7.43-7.34 (m, 1H), 7.18-7.06 (m, 1H), 5.23-5.14 (m, 1H), 5.01 (t, J=7.5 Hz, 1H), 4.37-4.28 (m, 1H), 2.48-2.22 (m, 5H), 2.13-1.91 (m, 3H), 1.91-1.67 (m, 2H), 1.65-1.52 (m, 1H), 1.48-1.33 (m, 4H), 1.32-1.16 (m, 3H), 1.16-1.00 (m, 2H). MS (ESI) m/z: [M+H]⁺ Found 568.3.

### Example 61

### 1-(Cyclopropylmethyl)-N—((S)-(4,4-difluorocyclohexyl)(6-((R)-1-(4,4,4-trifluorobutanamido)ethyl)-1H-benzo[d]imidazol-2-yl)methyl)-1H-pyrazole-4-carboxamide

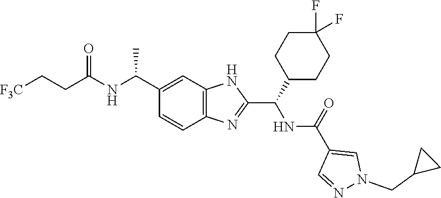 [SMILES: C[C@@H](NC(=O)CCC(F)(F)F)c1ccc2nc([C@@H](NC(=O)c3cnn(CC4CC4)c3)C3CCC(F)(F)CC3)[nH]c2c1]

The title compound was prepared as described for the synthesis of Example 58, using 1-(cyclopropylmethyl)-1H-pyrazole-4-carboxylic acid in place of 1-methyl-5-(trifluoromethyl)-1H-pyrazole-4-carboxylic acid to provide the title compound. ¹H NMR (400 MHz, DMSO-d₆) δ 12.29 (d, J=3.6 Hz, 1H), 8.52-8.37 (m, 2H), 8.35-8.29 (m, 1H), 7.94 (d, J=0.7 Hz, 1H), 7.53-7.46 (m, 1H), 7.40-7.33 (m, 1H), 7.15-7.06 (m, 1H), 5.21-5.11 (m, 1H), 5.07-4.94 (m, 1H), 3.97 (d, J=7.1 Hz, 2H), 2.48-2.30 (m, 4H), 2.30-2.17 (m, 1H), 2.12-1.88 (m, 3H), 1.88-1.66 (m, 2H), 1.60-1.49 (m, 1H), 1.38 (d, J=6.9 Hz, 4H), 1.31-1.15 (m, 2H), 0.59-0.49 (m, 2H), 0.40-0.31 (m, 2H). MS (ESI) m/z: [M+H]⁺ Found 581.3.

### Example 62

### 2-(Cyclobutylmethyl)-N—((S)-(4,4-difluorocyclohexyl)(6-((R)-1-(4,4,4-trifluorobutanamido)ethyl)-1H-benzo[d]imidazol-2-yl)methyl)-2H-1,2,3-triazole-4-carboxamide

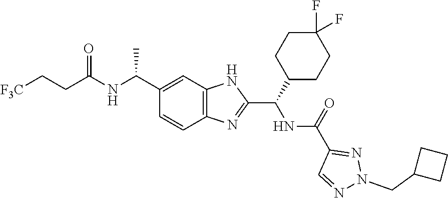 [SMILES: C[C@@H](NC(=O)CCC(F)(F)F)c1ccc2nc([C@@H](NC(=O)c3cnn(CC4CCC4)n3)C3CCC(F)(F)CC3)[nH]c2c1]

The title compound was prepared as described for the synthesis of Example 58, using 2-(cyclobutylmethyl)-2H-1,2,3-triazole-4-carboxylic acid (Intermediate 90) in place of 1-methyl-5-(trifluoromethyl)-1H-pyrazole-4-carboxylic acid and purified by basic preparative HPLC (ISCO ACCQ Prep, Gemini Prep NX-C18, 5 μm, 21.5×150 mm column, 10-70% acetonitrile/20 mM ammonium hydroxide (aqueous) over 20 min) to provide the title compound. ¹H NMR (400 MHz, DMSO-d₆) δ 12.35 (s, 1H), 8.65 (d, J=8.8 Hz, 1H), 8.42 (s, 1H), 8.19 (s, 1H), 7.59-7.34 (m, 2H), 7.12 (d, J=8.3 Hz, 1H), 5.18 (t, J=8.6 Hz, 1H), 5.06-4.95 (m, 1H), 4.51 (d, J=7.3 Hz, 2H), 2.94-2.81 (m, 1H), 2.49-2.35 (m, 3H), 2.31-2.17 (m, 1H), 2.09-1.66 (m, 11H), 1.59-1.47 (m, 1H), 1.42-1.18 (m, 6H). MS (ESI) m/z: [M+H]⁺ Found 596.3.

### Example 63

### 1-(Cyclobutylmethyl)-N—((S)-(4,4-difluorocyclohexyl)(6-((R)-1-(4,4,4-trifluorobutanamido)ethyl)-1H-benzo[d]imidazol-2-yl)methyl)-1H-1,2,3-triazole-5-carboxamide

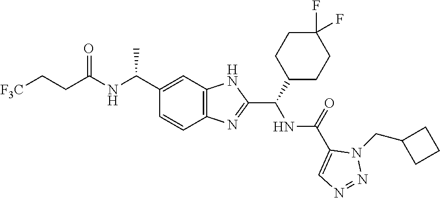 [SMILES: C[C@@H](NC(=O)CCC(F)(F)F)c1ccc2nc([C@@H](NC(=O)c3cnnn3CC3CCC3)C3CCC(F)(F)CC3)[nH]c2c1]

The title compound was prepared as described for the synthesis of Example 58, using 1-(cyclobutylmethyl)-1H-1,2,3-triazole-5-carboxylic acid (Intermediate 415) in place of 1-methyl-5-(trifluoromethyl)-1H-pyrazole-4-carboxylic acid and purified a second time by basic preparative HPLC (ISCO ACCQ Prep, Gemini Prep NX-C18, 5 μm, 21.5×150 mm column, 10-70% acetonitrile/20 mM ammonium hydroxide (aqueous) over 20 min) to provide the title compound. ¹H NMR (400 MHz, DMSO-d₆) δ 12.38 (s, 1H), 9.29 (d, J=8.4 Hz, 1H), 8.48-8.38 (m, 1H), 8.36 (s, 1H), 7.51 (d, J=6.8 Hz, 1H), 7.45-7.33 (m, 1H), 7.20-7.06 (m, 1H), 5.15 (t, J=8.2 Hz, 1H), 5.08-4.95 (m, 1H), 4.73-4.59 (m, 2H), 2.79-2.65 (m, 1H), 2.47-2.21 (m, 4H), 2.13-1.91 (m, 3H), 1.91-1.62 (m, 9H), 1.62-1.51 (m, 1H), 1.47-1.19 (m, 5H). MS (ESI) m/z: [M+H]⁺ Found 596.3.

### Example 64

### 2-((3,3-Difluorocyclobutyl)methyl)-N—((S)-(4,4-difluorocyclohexyl)(6-((R)-1-(4,4,4-trifluorobutanamido)ethyl)-1H-benzo[d]imidazol-2-yl)methyl)-2H-1,2,3-triazole-4-carboxamide

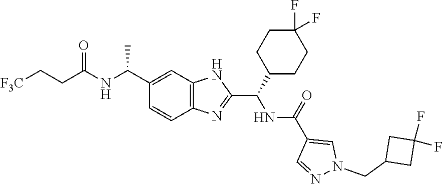 [SMILES: C[C@@H](NC(=O)CCC(F)(F)F)c1ccc2nc([C@@H](NC(=O)c3cnn(CC4CC(F)(F)C4)c3)C3CCC(F)(F)CC3)[nH]c2c1]

The title compound was prepared as described for the synthesis of Example 58, using 2-((3,3-difluorocyclobutyl)methyl)-2H-1,2,3-triazole-4-carboxylic acid (Intermediate 94) in place of 1-methyl-5-(trifluoromethyl)-1H-pyrazole-4-carboxylic acid, DMF in place of CH₃CN and purified by basic preparative HPLC (ISCO ACCQ Prep, Gemini Prep NX-C18, 5 μm, 21.5×150 mm column, 10-70% acetonitrile/20 mM ammonium hydroxide (aqueous) over 20 min) to provide the title compound. ¹H NMR (400 MHz, DMSO-d₆) δ 12.35 (s, 1H), 8.70 (d, J=8.8 Hz, 1H), 8.43 (d, J=7.8 Hz, 1H), 8.24 (s, 1H), 7.59-7.34 (m, 2H), 7.12 (d, J=8.4 Hz, 1H), 5.19 (t, J=8.6 Hz, 1H), 5.06-4.95 (m, 1H), 4.64 (d, J=6.5 Hz, 2H), 2.81-2.62 (m, 3H), 2.47-2.32 (m, 4H), 2.31-2.18 (m, 1H), 2.13-1.65 (m, 6H), 1.53 (d, J=13.5 Hz, 1H), 1.44-1.12 (m, 6H). MS (ESI) m/z: [M+H]⁺ Found 632.3.

### Example 65

### 1-((3,3-Difluorocyclobutyl)methyl)-N—((S)-(4,4-difluorocyclohexyl)(6-((R)-1-(4,4,4-trifluorobutanamido)ethyl)-1H-benzo[d]imidazol-2-yl)methyl)-1H-1,2,3-triazole-4-carboxamide

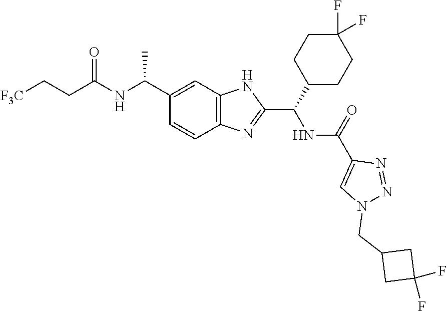 [SMILES: C[C@@H](NC(=O)CCC(F)(F)F)c1ccc2nc([C@@H](NC(=O)c3cn(CC4CC(F)(F)C4)nn3)C3CCC(F)(F)CC3)[nH]c2c1]

The title compound was prepared as described for the synthesis of Example 58, using 1-((3,3-difluorocyclobutyl)methyl)-1H-1,2,3-triazole-4-carboxylic acid (Intermediate 96) in place of 1-methyl-5-(trifluoromethyl)-1H-pyrazole-4-carboxylic acid, DMF in place of CH₃CN and purified by basic preparative HPLC (ISCO ACCQ Prep, Gemini Prep NX-C18, 5 μm, 21.5×150 mm column, 10-70% acetonitrile/20 mM ammonium hydroxide (aqueous) over 20 min) to provide the title compound. ¹H NMR (400 MHz, DMSO-d₆) δ 12.34 (s, 1H), 8.73-8.62 (m, 2H), 8.42 (d, J=8.0 Hz, 1H), 7.60-7.32 (m, 2H), 7.12 (dd, J=8.3, 1.6 Hz, 1H), 5.20 (t, J=8.6 Hz, 1H), 5.06-4.94 (m, 1H), 4.57 (d, J=6.4 Hz, 2H), 2.74-2.59 (m, 3H), 2.48-2.33 (m, 6H), 2.28-2.15 (m, 1H), 2.09-1.65 (m, 5H), 1.58-1.46 (m, 1H), 1.43-1.17 (m, 5H). MS (ESI) m/z: [M+H]⁺ Found 632.3.

### Example 66

### 1-((3,3-Difluorocyclobutyl)methyl)-N—((S)-(4,4-difluorocyclohexyl)(6-((R)-1-(4,4,4-trifluorobutanamido)ethyl)-1H-benzo[d]imidazol-2-yl)methyl)-1H-1,2,3-triazole-5-carboxamide

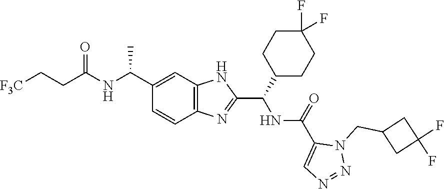 [SMILES: C[C@@H](NC(=O)CCC(F)(F)F)c1ccc2nc([C@@H](NC(=O)c3cnnn3CC3CC(F)(F)C3)C3CCC(F)(F)CC3)[nH]c2c1]

The title compound was prepared as described for the synthesis of Example 58, using 1-((3,3-difluorocyclobutyl)methyl)-1H-1,2,3-triazole-5-carboxylic acid (Intermediate 95) in place of 1-methyl-5-(trifluoromethyl)-1H-pyrazole-4-carboxylic acid, DMF in place of CH₃CN and purified a second time by basic preparative HPLC (ISCO ACCQ Prep, Gemini Prep NX-C18, 5 μm, 21.5×150 mm column, 10-70% acetonitrile/20 mM ammonium hydroxide (aqueous) over 20 min) to provide the title compound. ¹H NMR (400 MHz, DMSO-d₆) δ 12.38 (s, 1H), 9.35 (s, 1H), 8.51-8.22 (m, 2H), 7.59-7.30 (m, 2H), 7.26-6.94 (m, 1H), 5.20-5.10 (m, 1H), 5.06-4.95 (m, 1H), 4.81-4.73 (m, 2H), 2.74-2.54 (m, 3H), 2.54-2.21 (m, 7H), 2.13-1.91 (m, 3H), 1.91-1.67 (m, 2H), 1.62-1.52 (m, 1H), 1.47-1.14 (m, 5H). MS (ESI) m/z: [M+H]⁺ Found 632.3.

### Example 67

### N—((S)-(4,4-Difluorocyclohexyl)(6-((R)-1-(4,4,4-trifluorobutanamido)ethyl)-1H-benzo[d]imidazol-2-yl)methyl)-2-(3,3-dimethylbutyl)-2H-1,2,3-triazole-4-carboxamide

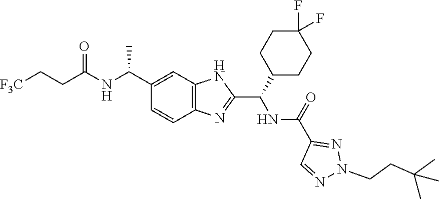 [SMILES: C[C@@H](NC(=O)CCC(F)(F)F)c1ccc2nc([C@@H](NC(=O)c3cnn(CCC(C)(C)C)n3)C3CCC(F)(F)CC3)[nH]c2c1]

The title compound was prepared as described for the synthesis of Example 58, using 2-(3,3-dimethylbutyl)-2H-1,2,3-triazole-4-carboxylic acid (Intermediate 83) in place of 1-methyl-5-(trifluoromethyl)-1H-pyrazole-4-carboxylic acid, DMF in place of CH₃CN and purified a second time by basic preparative HPLC (ISCO ACCQ Prep, Gemini Prep NX-C18, 5 μm, 21.5×150 mm column, 10-70% acetonitrile/20 mM ammonium hydroxide (aqueous) over 20 min) to provide the title compound. ¹H NMR (400 MHz, DMSO-d₆) δ 12.34 (s, 1H), 8.67 (d, J=8.9 Hz, 1H), 8.43 (s, 1H), 8.19 (s, 1H), 7.58-7.33 (m, 2H), 7.12 (d, J=8.4 Hz, 1H), 5.19 (t, J=8.7 Hz, 1H), 5.06-4.94 (m, 1H), 4.53-4.43 (m, 2H), 2.49-2.32 (m, 4H), 2.31-2.17 (m, 1H), 2.11-1.87 (m, 3H), 1.87-1.65 (m, 4H), 1.58-1.46 (m, 1H), 1.42-1.15 (m, 5H), 0.93 (s, 9H). MS (ESI) m/z: [M+H]⁺ Found 612.3.

### Example 68

### N—((S)-(4,4-Difluorocyclohexyl)(6-((R)-1-(4,4,4-trifluorobutanamido)ethyl)-1H-benzo[d]imidazol-2-yl)methyl)-1-(3,3-dimethylbutyl)-1H-1,2,3-triazole-5-carboxamide

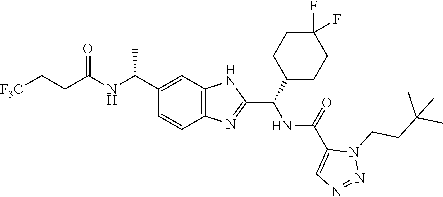 [SMILES: C[C@@H](NC(=O)CCC(F)(F)F)c1ccc2nc([C@@H](NC(=O)c3cnnn3CCC(C)(C)C)C3CCC(F)(F)CC3)[nH]c2c1]

The title compound was prepared as described for the synthesis of Example 58, using 1-(3,3-dimethylbutyl)-1H-1,2,3-triazole-5-carboxylic acid (Intermediate 84) in place of 1-methyl-5-(trifluoromethyl)-1H-pyrazole-4-carboxylic acid, DMF in place of CH₃CN and purified a second time by basic preparative HPLC (ISCO ACCQ Prep, Gemini Prep NX-C18, 5 μm, 21.5×150 mm column, 10-70% acetonitrile/20 mM ammonium hydroxide (aqueous) over 20 min) to provide the title compound. ¹H NMR (600 MHz, DMSO-d₆) δ 12.38 (s, 1H), 9.29 (d, J=8.5 Hz, 1H), 8.43 (dd, J=17.7, 8.0 Hz, 1H), 8.36 (s, 1H), 7.55-7.47 (m, 1H), 7.42-7.34 (m, 1H), 7.12 (dd, J=16.2, 8.2 Hz, 1H), 5.21-5.13 (m, 1H), 5.05-4.95 (m, 1H), 4.71-4.57 (m, 2H), 2.49-2.32 (m, 4H), 2.31-2.22 (m, 1H), 2.11-1.91 (m, 3H), 1.88-1.72 (m, 2H), 1.65-1.51 (m, 3H), 1.44-1.33 (m, 4H), 1.33-1.21 (m, 1H), 0.87 (s, 9H). MS (ESI) m/z: [M+H]⁺ Found 612.3.

### Example 69

### N—((S)-(4,4-Difluorocyclohexyl)(6-((R)-1-(4,4,4-trifluorobutanamido)ethyl)-1H-benzo[d]imidazol-2-yl)methyl)-2-((R*)-3,3,3-trifluoro-2-methylpropyl)-2H-1,2,3-triazole-4-carboxamide

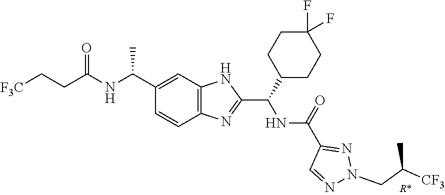 [SMILES: C[C@H](Cn1ncc(C(=O)N[C@H](c2nc3ccc([C@@H](C)NC(=O)CCC(F)(F)F)cc3[nH]2)C2CCC(F)(F)CC2)n1)C(F)(F)F]

### Example 70

### N—((S)-(4,4-Difluorocyclohexyl)(6-((R)-1-(4,4,4-trifluorobutanamido)ethyl)-1H-benzo[d]imidazol-2-yl)methyl)-2-((S*)-3,3,3-trifluoro-2-methylpropyl)-2H-1,2,3-triazole-4-carboxamide

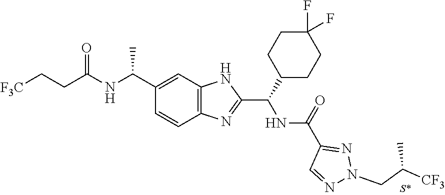 [SMILES: C[C@@H](NC(=O)CCC(F)(F)F)c1ccc2nc([C@@H](NC(=O)c3cnn(C[C@H](C)C(F)(F)F)n3)C3CCC(F)(F)CC3)[nH]c2c1]

The title compounds were prepared as described for the synthesis of Example 58, using 2-(3,3,3-trifluoro-2-methylpropyl)-2H-1,2,3-triazole-4-carboxylic acid (Intermediate 87) in place of 1-methyl-5-(trifluoromethyl)-1H-pyrazole-4-carboxylic acid, DMF in place of CH₃CN and purified a second time by basic preparative HPLC (ISCO ACCQ Prep, Gemini Prep NX-C18 5 μm column 21.5×150 mm, gradient 10-70% acetonitrile/20 mM ammonium hydroxide (aqueous) over 20 min) to provide the title compounds as a mixture of diastereomers. The diastereomers were separated by chiral SFC (Chiralcel OD-H, 5 μm, 250×21.2 mm, mobile phase: 80% CO₂, 20% mixture of 90:10 ACN/MeOH) to provide Example 69 as the first eluting fraction and Example 70 as the second eluting fraction. Example 69: ¹H NMR (400 MHz, DMSO-d₆) δ 12.35 (s, 1H), 8.73 (d, J=8.8 Hz, 1H), 8.48-8.36 (m, 1H), 8.28 (s, 1H), 7.55-7.46 (m, 1H), 7.45-7.34 (m, 1H), 7.18-7.06 (m, 1H), 5.24-5.14 (m, 1H), 5.09-4.95 (m, 1H), 4.82-4.72 (m, 1H), 4.67-4.55 (m, 1H), 3.27-3.11 (m, 1H), 2.48-2.32 (m, 4H), 2.32-2.17 (m, 1H), 2.11-1.65 (m, 5H), 1.58-1.47 (m, 1H), 1.43-1.19 (m, 5H), 1.15-0.99 (m, 3H). MS (ESI) m/z: [M+H]⁺ Found 638.3. Example 70: ¹H NMR (400 MHz, DMSO-d₆) δ 12.36 (s, 1H), 8.74 (d, J=8.8 Hz, 1H), 8.43 (d, J=7.6 Hz, 1H), 8.28 (s, 1H), 7.58-7.31 (m, 2H), 7.19-7.06 (m, 1H), 5.19 (t, J=8.7 Hz, 1H), 5.06-4.95 (m, 1H), 4.82-4.56 (m, 2H), 3.26-3.12 (m, 1H), 2.48-2.18 (m, 5H), 2.12-1.66 (m, 5H), 1.59-1.45 (m, 1H), 1.42-1.18 (m, 5H), 1.15-1.06 (m, 3H). MS (ESI) m/z: [M+H]⁺ Found 638.3.

### Example 71

### N—((S)-(4,4-Difluorocyclohexyl)(6-((R)-1-(4,4,4-trifluorobutanamido)ethyl)-1H-benzo[d]imidazol-2-yl)methyl)-1-((R*)-3,3,3-trifluoro-2-methylpropyl)-1H-1,2,3-triazole-5-carboxamide

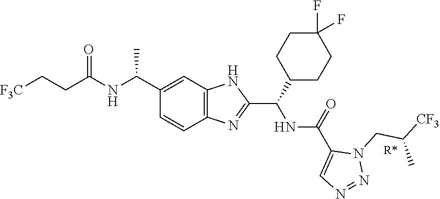 [SMILES: C[C@H](Cn1nncc1C(=O)N[C@H](c1nc2ccc([C@@H](C)NC(=O)CCC(F)(F)F)cc2[nH]1)C1CCC(F)(F)CC1)C(F)(F)F]

### Example 72

### N—((S)-(4,4-Difluorocyclohexyl)(6-((R)-1-(4,4,4-trifluorobutanamido)ethyl)-1H-benzo[d]imidazol-2-yl)methyl)-1-((S*)-3,3,3-trifluoro-2-methylpropyl)-1H-1,2,3-triazole-5-carboxamide

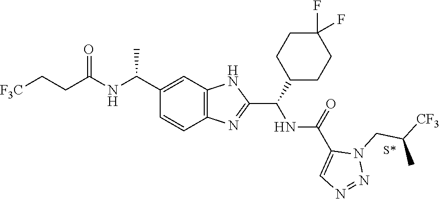 [SMILES: C[C@@H](NC(=O)CCC(F)(F)F)c1ccc2nc([C@@H](NC(=O)c3cnnn3C[C@H](C)C(F)(F)F)C3CCC(F)(F)CC3)[nH]c2c1]

The title compounds were prepared as described for the synthesis of Example 58, using 1-(3,3,3-trifluoro-2-methylpropyl)-1H-1,2,3-triazole-5-carboxylic acid (Intermediate 88) in place of 1-methyl-5-(trifluoromethyl)-1H-pyrazole-4-carboxylic acid, DMF in place of CH₃CN and purified a second time by basic preparative HPLC (ISCO ACCQ Prep, Gemini Prep NX-C18, 5 μm, 21.5×150 mm column, 10-70% acetonitrile/20 mM ammonium hydroxide (aqueous) over 20 min) to provide the title compounds as a mixture of diastereomers. The diastereomers were separated by chiral SFC (Stationary phase: CHIRALPAK AD-H, 5 μm, 250×21.2 mm, mobile phase: 90% CO₂, 10% mixture of 75:25 i-PrOH/heptane) to provide Example 71 as the first eluting fraction and Example 72 as the second eluting fraction. Example 71: ¹H NMR (400 MHz, DMSO-d₆) δ 12.38 (s, 1H), 9.45-9.30 (m, 1H), 8.49 (d, J=1.1 Hz, 1H), 8.47-8.36 (m, 1H), 7.55-7.48 (m, 1H), 7.42-7.35 (m, 1H), 7.17-7.08 (m, 1H), 5.24-5.13 (m, 1H), 5.08-4.90 (m, 2H), 4.78-4.65 (m, 1H), 3.19-3.02 (m, 1H), 2.49-2.20 (m, 5H), 2.12-1.90 (m, 3H), 1.90-1.67 (m, 2H), 1.60-1.51 (m, 1H), 1.43-1.35 (m, 3H), 1.32-1.20 (m, 2H), 1.02-0.95 (m, 3H). MS (ESI) m/z: [M+H]⁺ Found 638.3. Example 72: ¹H NMR (400 MHz, DMSO-d₆) δ 12.39 (s, 1H), 9.38 (d, J=8.4 Hz, 1H), 8.53-8.36 (m, 2H), 7.57-7.48 (m, 1H), 7.44-7.34 (m, 1H), 7.18-7.07 (m, 1H), 5.21-5.12 (m, 1H), 5.06-4.93 (m, 2H), 4.73-4.61 (m, 1H), 3.18-3.01 (m, 1H), 2.49-2.21 (m, 5H), 2.12-1.90 (m, 3H), 1.90-1.67 (m, 2H), 1.61-1.47 (m, 1H), 1.41-1.35 (m, 3H), 1.31-1.21 (m, 2H), 1.03-0.96 (m, 3H). MS (ESI) m/z: [M+H]⁺ Found 638.3.

### Example 73

### N—((S)-(4,4-Difluorocyclohexyl)(6-((R)-1-(4,4,4-trifluorobutanamido)ethyl)-1H-benzo[d]imidazol-2-yl)methyl)-2-(3,3-difluoropropyl)-2H-1,2,3-triazole-4-carboxamide

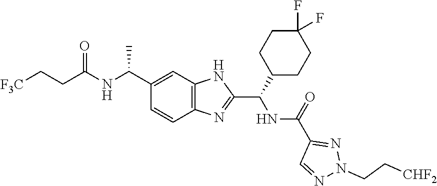 [SMILES: C[C@@H](NC(=O)CCC(F)(F)F)c1ccc2nc([C@@H](NC(=O)c3cnn(CCC(F)F)n3)C3CCC(F)(F)CC3)[nH]c2c1]

The title compound was prepared as described for the synthesis of Example 58, using 2-(3,3-difluoropropyl)-2H-1,2,3-triazole-4-carboxylic acid (Intermediate 99) in place of 1-methyl-5-(trifluoromethyl)-1H-pyrazole-4-carboxylic acid to provide the title compound. ¹H NMR (400 MHz, DMSO-d₆) δ 12.34 (s, 1H), 8.71 (d, J=8.9 Hz, 1H), 8.47-8.37 (m, 1H), 8.23 (s, 1H), 7.54-7.49 (m, 1H), 7.44-7.37 (m, 1H), 7.17-7.08 (m, 1H), 6.40-6.05 (m, 1H), 5.23-5.15 (m, 1H), 5.06-4.96 (m, 1H), 4.66 (t, J=6.9 Hz, 2H), 2.48-2.31 (m, 5H), 2.31-2.18 (m, 1H), 2.10-1.66 (m, 5H), 1.58-1.48 (m, 1H), 1.42-1.17 (m, 6H). MS (ESI) m/z: [M+H]⁺ Found 606.2.

### Example 74

### N—((S)-(4,4-Difluorocyclohexyl)(6-((R)-1-(4,4,4-trifluorobutanamido)ethyl)-1H-benzo[d]imidazol-2-yl)methyl)-1-(3,3-difluoropropyl)-1H-1,2,3-triazole-5-carboxamide

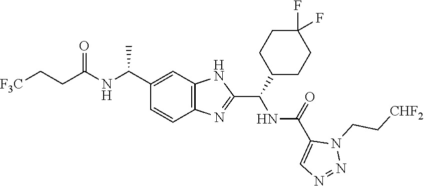 [SMILES: C[C@@H](NC(=O)CCC(F)(F)F)c1ccc2nc([C@@H](NC(=O)c3cnnn3CCC(F)F)C3CCC(F)(F)CC3)[nH]c2c1]

The title compound was prepared as described for the synthesis of Example 58, using 1-(3,3-difluoropropyl)-1H-1,2,3-triazole-5-carboxylic acid (Intermediate 100) in place of 1-methyl-5-(trifluoromethyl)-1H-pyrazole-4-carboxylic acid to provide the title compound. ¹H NMR (400 MHz, DMSO-d₆) δ 12.38 (s, 1H), 9.37-9.26 (m, 1H), 8.51-8.36 (m, 2H), 7.55-7.47 (m, 1H), 7.44-7.34 (m, 1H), 7.18-7.05 (m, 1H), 6.18-6.12 (m, 1H), 5.22-5.11 (m, 1H), 5.09-4.94 (m, 1H), 4.81 (t, J=7.1 Hz, 2H), 2.48-2.20 (m, 7H), 2.15-1.91 (m, 3H), 1.90-1.68 (m, 2H), 1.62-1.51 (m, 1H), 1.46-1.19 (m, 5H). MS (ESI) m/z: [M+H]⁺ Found 606.3.

### Example 75

### 3-Cyclopropyl-N—((S)-(4,4-difluorocyclohexyl)(6-((R)-1-(4,4,4-trifluorobutanamido)ethyl)-1H-benzo[d]imidazol-2-yl)methyl)-1-(2,2,2-trifluoroethyl)-1H-pyrazole-4-carboxamide

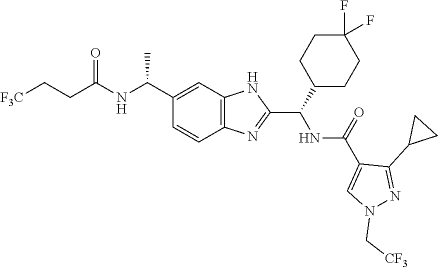 [SMILES: C[C@@H](NC(=O)CCC(F)(F)F)c1ccc2nc([C@@H](NC(=O)c3cn(CC(F)(F)F)nc3C3CC3)C3CCC(F)(F)CC3)[nH]c2c1]

The title compound was prepared as described for the synthesis of Example 58, using 3-cyclopropyl-1-(2,2,2-trifluoroethyl)-1H-pyrazole-4-carboxylic acid in place of 1-methyl-5-(trifluoromethyl)-1H-pyrazole-4-carboxylic acid to provide the title compound. ¹H NMR (400 MHz, DMSO-d₆) δ 12.27 (d, J=4.6 Hz, 1H), 8.48-8.30 (m, 2H), 7.90 (d, J=1.5 Hz, 1H), 7.53-7.47 (m, 1H), 7.41-7.35 (m, 1H), 7.17-7.07 (m, 1H), 5.21-5.08 (m, 3H), 5.08-4.94 (m, 1H), 2.48-2.31 (m, 4H), 2.28-2.14 (m, 1H), 2.11-1.66 (m, 6H), 1.61-1.50 (m, 1H), 1.38 (d, J=7.0 Hz, 4H), 1.33-1.17 (m, 1H), 1.05-0.89 (m, 2H), 0.82-0.69 (m, 2H). MS (ESI) m/z: [M+H]⁺ Found 649.3.

### Example 76

### N—((R)-1-(6-((R)-Cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-1-(ethyl-d₅)-1H-pyrazole-5-carboxamide

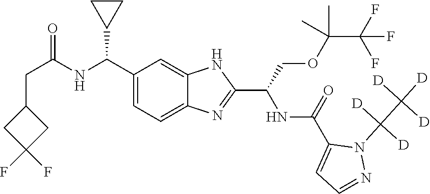 [SMILES: [2H]C([2H])([2H])C([2H])([2H])n1nccc1C(=O)N[C@@H](COC(C)(C)C(F)(F)F)c1nc2ccc([C@H](NC(=O)CC3CC(F)(F)C3)C3CC3)cc2[nH]1]

A solution of N—((R)-(2-((R)-1-amino-2-((1,1,1-trifluoro-2-methylpropan-2-yl)oxy)ethyl)-1H-benzo[d]imidazol-6-yl)(cyclopropyl)methyl)-2-(3,3-difluorocyclobutyl)acetamide (9.9 mg, 0.0203 mmol, Intermediate 197), 1-(ethyl-d₅)-1H-pyrazole-5-carboxylic acid (3.2 mg, 0.022 mmol, Intermediate 413), DIPEA (0.01 mL, 0.057 mmol) and HOBt (3.0 mg, 0.022 mmol) in MeCN (0.5 mL) was heated to 45° C. and then EDCI (4.3 mg, 0.022 mmol) was added. The reaction was stirred at 45° C. for 2 h then diluted with EtOAc (5 mL), washed with saturated aqueous ammonium chloride, saturated aqueous sodium bicarbonate, dried over anhydrous Na₂SO₄, filtered and condensed. Purification by silica gel chromatography (10-100% (10% MeOH in EtOAc)/hexanes) provided the title compound. ¹H NMR (500 MHz, DMSO-d₆) δ 12.62-12.03 (m, 1H), 9.16-8.68 (m, 1H), 8.65-8.26 (m, 1H), 7.62-7.46 (m, 2H), 7.44-7.34 (m, 1H), 7.22-7.10 (m, 1H), 7.00-6.90 (m, 1H), 5.45-5.37 (m, 1H), 4.42-4.29 (m, 1H), 4.22-4.13 (m, 1H), 4.05-3.94 (m, 1H), 2.72-2.55 (m, 2H), 2.41-2.20 (m, 5H), 1.33 (s, 6H), 1.22-1.06 (m, 1H), 0.56-0.40 (m, 2H), 0.37-0.24 (m, 2H). MS (ESI) m/z: [M+H]⁺ Found 616.3.

### Example 77

### N—((R)-1-(6-((R)-Cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-3-methylisoxazole-4-carboxamide

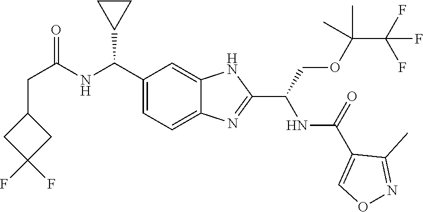 [SMILES: Cc1nocc1C(=O)N[C@@H](COC(C)(C)C(F)(F)F)c1nc2ccc([C@H](NC(=O)CC3CC(F)(F)C3)C3CC3)cc2[nH]1]

To a solution of N—((R)-(2-((R)-1-amino-2-((1,1,1-trifluoro-2-methylpropan-2-yl)oxy)ethyl)-1H-benzo[d]imidazol-6-yl)(cyclopropyl)methyl)-2-(3,3-difluorocyclobutyl)acetamide (43 mg, 0.074 mmol, Intermediate 197) in EtOAc (1 mL) was added 3-methylisoxazole-4-carboxylic acid (27 mg, 0.21 mmol), DIPEA (0.066 mL, 0.38 mmol) and T3P® (0.099 mL, 0.13 mmol). The resulting mixture was stirred at rt for 2.5 h and then quenched with 0.2 M aqueous HCl (10 mL). The mixture was extracted with EtOAc (2×10 mL) then the combined organic layers were washed with saturated aqueous NaHCO₃ and brine, dried over anhydrous Na₂SO₄, filtered and condensed. Purification by silica gel chromatography (10-100% (10% MeOH in EtOAc)/hexanes) provided the title compound. ¹H NMR (500 MHz, DMSO-d₆) δ 12.52-12.19 (m, 1H), 9.41-9.28 (m, 1H), 8.95-8.80 (m, 1H), 8.55-8.34 (m, 1H), 7.61-7.47 (m, 1H), 7.45-7.33 (m, 1H), 7.26-7.07 (m, 1H), 5.46-5.29 (m, 1H), 4.45-4.27 (m, 1H), 4.17-4.07 (m, 1H), 3.99-3.89 (m, 1H), 2.74-2.54 (m, 2H), 2.38-2.33 (m, 3H), 2.38-2.22 (m, 5H), 1.37-1.29 (m, 6H), 1.20-1.09 (m, 1H), 0.56-0.41 (m, 2H), 0.37-0.24 (m, 2H). MS (ESI) m/z: [M+H]⁺ Found 598.2.

### Example 78

### 2-((3-Cyanobicyclo[1.1.1]pentan-1-yl)methyl)-N—((S)-(4,4-difluorocyclohexyl)(5-((R)-1-(4,4,4-trifluorobutanamido)ethyl)-1H-benzo[d]imidazol-2-yl)methyl)-2H-1,2,3-triazole-4-carboxamide

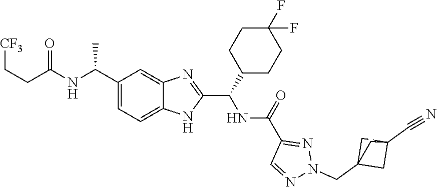 [SMILES: C[C@@H](NC(=O)CCC(F)(F)F)c1ccc2[nH]c([C@@H](NC(=O)c3cnn(CC45CC(C#N)(C4)C5)n3)C3CCC(F)(F)CC3)nc2c1]

A mixture of N—((R)-1-(2-((S)-amino(4,4-difluorocyclohexyl)methyl)-1H-benzo[d]imidazol-5-yl)ethyl)-4,4,4-trifluorobutanamide (51 mg, 0.1 mmol, Intermediate 4), 2-((3-cyanobicyclo[1.1.1]pentan-1-yl)methyl)-2H-1,2,3-triazole-4-carboxylic acid (29 mg, 0.13 mmol, Intermediate 103), HOBt (20 mg, 0.15 mmol), DIPEA (70 μL, 0.41 mmol), EDCI (28.3 mg, 0.15 mmol) and ACN (2.1 mL) was stirred at rt for 67.3 h. After this time, the mixture was concentrated to dryness and purified by silica gel chromatography (0-100% EtOAc/hexanes) followed by preparative HPLC (Boston Prime, C18, 250×50 mm, 5 μM, 10-100% MeCN/water with 20 mM NH₃) to provide the title compound as a white solid. ¹H NMR (500 MHz, CD₃OD) δ 8.08 (s, 1H), 7.53-7.49 (m, 2H), 7.23 (dd, J=8.6, 1.6 Hz, 1H), 5.26 (d, J=8.8 Hz, 1H), 5.11 (q, J=7.0 Hz, 1H), 4.60 (s, 2H), 2.56-2.38 (m, 4H), 2.34-2.24 (m, 1H), 2.21 (s, 6H), 2.15-2.05 (m, 1H), 2.05-1.95 (m, 2H), 1.90-1.66 (m, 2H), 1.59-1.51 (m, 1H), 1.50-1.43 (m, 4H), 1.43-1.33 (m, 1H). MS (ESI) m/z: [M+H]⁺ Found 633.3.

### Example 79

### 1-((3-Cyanobicyclo[1.1.1]pentan-1-yl)methyl)-N—((S)-(4,4-difluorocyclohexyl)(5-((R)-1-(4,4,4-trifluorobutanamido)ethyl)-1H-benzo[d]imidazol-2-yl)methyl)-1H-1,2,3-triazole-4-carboxamide

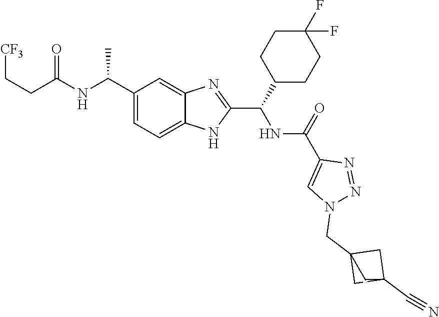 [SMILES: C[C@@H](NC(=O)CCC(F)(F)F)c1ccc2[nH]c([C@@H](NC(=O)c3cn(CC45CC(C#N)(C4)C5)nn3)C3CCC(F)(F)CC3)nc2c1]

The title compound was prepared as described in the synthesis of Example 78, using 1-((3-cyanobicyclo[1.1.1]pentan-1-yl)methyl)-1H-1,2,3-triazole-4-carboxylic acid (Intermediate 105) in place of 2-((3-cyanobicyclo[1.1.1]pentan-1-yl)methyl)-2H-1,2,3-triazole-4-carboxylic acid, and purified via silica gel chromatography (0-100% (10% MeOH in EtOAc)/DCM) to provide the title compound as a white solid. ¹H NMR (500 MHz, CD₃OD) δ 8.35 (s, 1H), 7.56-7.45 (m, 2H), 7.23 (dd, J=8.5, 1.6 Hz, 1H), 5.28 (d, J=8.6 Hz, 1H), 5.10 (q, J=7.0 Hz, 1H), 4.57 (s, 2H), 2.55-2.38 (m, 4H), 2.32-2.22 (m, 1H), 2.17 (s, 6H), 2.13-2.06 (m, 1H), 2.06-1.96 (m, 2H), 1.90-1.66 (m, 2H), 1.61-1.53 (m, 1H), 1.53-1.45 (m, 4H), 1.45-1.33 (m, 1H). MS (ESI) m/z: [M+H]⁺ Found 633.3.

### Example 80

### N—((S)-(4,4-Difluorocyclohexyl)(5-((R)-1-(4,4,4-trifluorobutanamido)ethyl)-1H-benzo[d]imidazol-2-yl)methyl)-1-((2,2-difluorocyclopropyl)methyl)-1H-1,2,3-triazole-5-carboxamide

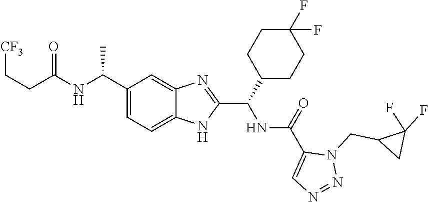 [SMILES: C[C@@H](NC(=O)CCC(F)(F)F)c1ccc2[nH]c([C@@H](NC(=O)c3cnnn3CC3CC3(F)F)C3CCC(F)(F)CC3)nc2c1]

A mixture of N—((R)-1-(2-((S)-amino(4,4-difluorocyclohexyl)methyl)-1H-benzo[d]imidazol-5-yl)ethyl)-4,4,4-trifluorobutanamide (25 mg, 0.05 mmol, Intermediate 4), 1-((2,2-difluorocyclopropyl)methyl)-1H-1,2,3-triazole-5-carboxylic acid (17.6 mg, 0.087 mmol, Intermediate 112), HOBt (11 mg, 0.081 mmol), DIPEA (40 μL, 0.23 mmol), EDCI (15.6 mg, 0.081 mmol) and ACN (1.2 mL) was stirred at rt for 17.25 h. After this time, the mixture was diluted with EtOAc, washed with 1 N aqueous NaOH followed by water (2×) and brine. The organic layer was dried over anhydrous MgSO₄, filtered, and concentrated to dryness. The residue was purified by silica gel chromatography (0-100% (10% MeOH/EtOAc)/hexanes) to provide the title compound as a white solid. ¹H NMR (400 MHz, CD₃OD) δ 8.31 (d, J=1.8 Hz, 1H), 7.51 (s, 2H), 7.30-7.18 (m, 1H), 5.20 (dd, J=8.7, 3.6 Hz, 1H), 5.11 (q, J=7.0 Hz, 1H), 4.91 (dd, J 14.4, 7.0 Hz, 1H), 4.74 (dd, J=14.4, 8.3 Hz, 1H), 2.56-2.38 (m, 4H), 2.37-2.22 (m, 2H), 2.18-1.96 (m, 3H), 1.93-1.67 (m, 2H), 1.64-1.24 (m, 8H). MS (ESI) m/z: [M+H]⁺ Found 618.3.

### Example 81

### N—((S)-(4,4-Difluorocyclohexyl)(5-((R)-1-(4,4,4-trifluorobutanamido)ethyl)-1H-benzo[d]imidazol-2-yl)methyl)-2-((2,2-difluorocyclopropyl)methyl)-2H-1,2,3-triazole-4-carboxamide

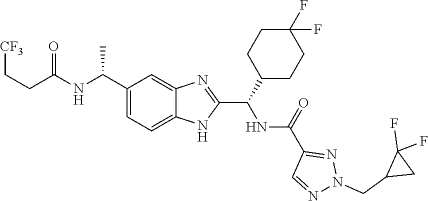 [SMILES: C[C@@H](NC(=O)CCC(F)(F)F)c1ccc2[nH]c([C@@H](NC(=O)c3cnn(CC4CC4(F)F)n3)C3CCC(F)(F)CC3)nc2c1]

The title compound was prepared as described in the synthesis of Example 80, using 2-((2,2-difluorocyclopropyl)methyl)-2H-1,2,3-triazole-4-carboxylic acid (Intermediate 114) in place of 1-((2,2-difluorocyclopropyl)methyl)-1H-1,2,3-triazole-5-carboxylic acid to provide the title compound as a white solid. ¹H NMR (500 MHz, CD₃OD) δ 8.10 (s, 1H), 7.67-7.35 (m, 3H), 7.30-7.11 (m, 1H), 5.27 (d, J=8.7 Hz, 1H), 5.11 (q, J=7.0 Hz, 1H), 4.69 (dd, J=14.4, 7.7 Hz, 1H), 4.62-4.52 (m, 1H), 2.58-2.37 (m, 4H), 2.37-2.18 (m, 2H), 2.16-1.90 (m, 3H), 1.90-1.69 (m, 2H), 1.69-1.60 (m, 1H), 1.60-1.27 (m, 6H). MS (ESI) m/z: [M+H]⁺ Found 618.3.

### Example 82

### N—((S)-(4,4-Difluorocyclohexyl)(5-((R)-1-(4,4,4-trifluorobutanamido)ethyl)-1H-benzo[d]imidazol-2-yl)methyl)-1-((2,2,3,3-tetrafluorocyclobutyl)methyl)-1H-1,2,3-triazole-5-carboxamide

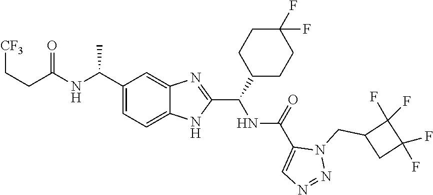 [SMILES: C[C@@H](NC(=O)CCC(F)(F)F)c1ccc2[nH]c([C@@H](NC(=O)c3cnnn3CC3CC(F)(F)C3(F)F)C3CCC(F)(F)CC3)nc2c1]

The title compound was prepared as described in the synthesis of Example 80, using 1-((2,2,3,3-tetrafluorocyclobutyl)methyl)-1H-1,2,3-triazole-5-carboxylic acid (Intermediate 117) in place of 1-((2,2-difluorocyclopropyl)methyl)-1H-1,2,3-triazole-5-carboxylic acid to provide the title compound as a white solid. ¹H NMR (500 MHz, CD₃OD) δ 8.31 (d, J=2.8 Hz, 1H), 7.69-7.33 (m, 2H), 7.24 (dd, J=8.4, 1.6 Hz, 1H), 5.21 (dd, J=8.7, 3.4 Hz, 1H), 5.11 (q, J=7.3 Hz, 1H), 5.08-5.00 (m, 1H), 4.95-4.86 (m, 1H), 3.54-3.38 (m, 1H), 2.72-2.57 (m, 1H), 2.55-2.37 (m, 5H), 2.35-2.24 (m, 1H), 2.17-1.96 (m, 3H), 1.91-1.68 (m, 2H), 1.63-1.55 (m, 1H), 1.55-1.45 (m, 4H), 1.44-1.33 (m, 1H). MS (ESI) m/z: [M+H]⁺ Found 668.3.

### Example 83

### N—((S)-(4,4-Difluorocyclohexyl)(5-((R)-1-(4,4,4-trifluorobutanamido)ethyl)-1H-benzo[d]imidazol-2-yl)methyl)-2-((2,2,3,3-tetrafluorocyclobutyl)methyl)-2H-1,2,3-triazole-4-carboxamide

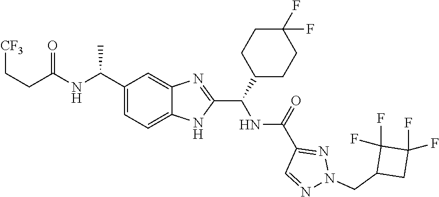 [SMILES: C[C@@H](NC(=O)CCC(F)(F)F)c1ccc2[nH]c([C@@H](NC(=O)c3cnn(CC4CC(F)(F)C4(F)F)n3)C3CCC(F)(F)CC3)nc2c1]

The title compound was prepared as described in the synthesis of Example 80, using 2-((2,2,3,3-tetrafluorocyclobutyl)methyl)-2H-1,2,3-triazole-4-carboxylic acid (Intermediate 119) in place of 1-((2,2-difluorocyclopropyl)methyl)-1H-1,2,3-triazole-5-carboxylic acid to provide the title compound as a white solid. ¹H NMR (500 MHz, CD₃OD) δ 8.09 (s, 1H), 7.59-7.39 (m, 2H), 7.24 (dd, J=8.4, 1.7 Hz, 1H), 5.26 (d, J=8.7 Hz, 1H), 5.11 (q, J=6.8 Hz, 1H), 4.89-4.82 (m, 1H), 4.69 (dd, J=14.2, 7.4 Hz, 1H), 3.60-3.41 (m, 1H), 2.85-2.68 (m, 1H), 2.60-2.35 (m, 5H), 2.35-2.21 (m, 1H), 2.17-1.94 (m, 3H), 1.90-1.65 (m, 2H), 1.61-1.51 (m, 1H), 1.51-1.45 (m, 4H), 1.45-1.33 (m, 1H). MS (ESI) m/z: [M+H]⁺ Found 668.3.

### Example 84

### N—((S)-(4,4-Difluorocyclohexyl)(5-((R)-1-(4,4,4-trifluorobutanamido)ethyl)-1H-benzo[d]imidazol-2-yl)methyl)-1-((2,2,3,3-tetrafluorocyclobutyl)methyl)-1H-1,2,3-triazole-4-carboxamide

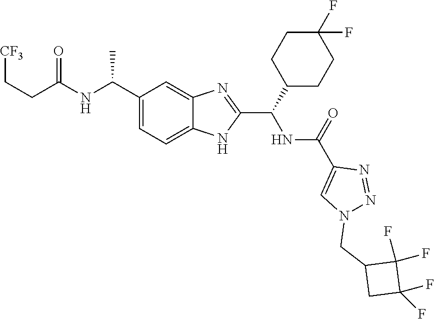 [SMILES: C[C@@H](NC(=O)CCC(F)(F)F)c1ccc2[nH]c([C@@H](NC(=O)c3cn(CC4CC(F)(F)C4(F)F)nn3)C3CCC(F)(F)CC3)nc2c1]

The title compound was prepared as described in the synthesis of Example 80, using 1-((2,2,3,3-tetrafluorocyclobutyl)methyl)-1H-1,2,3-triazole-4-carboxylic acid (Intermediate 121) in place of 1-((2,2-difluorocyclopropyl)methyl)-1H-1,2,3-triazole-5-carboxylic acid to provide the title compound as a white solid. ¹H NMR (500 MHz, CD₃OD) δ 8.43 (s, 1H), 7.62-7.39 (m, 2H), 7.23 (dd, J=8.4, 1.6 Hz, 1H), 5.28 (d, J=8.6 Hz, 1H), 5.11 (q, J=7.0 Hz, 1H), 4.82-4.77 (m, 1H), 4.70 (dd, J=14.4, 7.1 Hz, 1H), 3.54-3.39 (m, 1H), 2.86-2.68 (m, 1H), 2.60-2.35 (m, 5H), 2.33-2.20 (m, 1H), 2.16-1.95 (m, 3H), 1.91-1.66 (m, 2H), 1.63-1.50 (m, 2H), 1.49 (d, J=7.0 Hz, 3H), 1.45-1.33 (m, 1H). MS (ESI) m/z: [M+H]⁺ Found 668.3.

### Example 85

### N—((S)-(4,4-Difluorocyclohexyl)(5-((R)-1-(4,4,4-trifluorobutanamido)ethyl)-1H-benzo[d]imidazol-2-yl)methyl)-1-(1,1-difluoropropan-2-yl)-1H-1,2,3-triazole-4-carboxamide

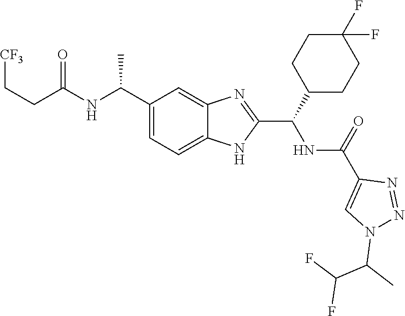 [SMILES: CC(C(F)F)n1cc(C(=O)N[C@H](c2nc3cc([C@@H](C)NC(=O)CCC(F)(F)F)ccc3[nH]2)C2CCC(F)(F)CC2)nn1]

The title compound was prepared as described in the synthesis of Example 78, using 1-(1,1-difluoropropan-2-yl)-1H-1,2,3-triazole-4-carboxylic acid (Intermediate 132) in place of 2-((3-cyanobicyclo[1.1.1]pentan-1-yl)methyl)-2H-1,2,3-triazole-4-carboxylic acid, and purified via silica gel chromatography (0-100% (10% MeOH in EtOAc)/DCM) to provide the title compound as a white solid. ¹H NMR (400 MHz, CD₃OD) δ 8.52 (s, 1H), 7.61-7.38 (m, 2H), 7.24 (dd, J 8.4, 1.6 Hz, 1H), 6.42-6.01 (m, 1H), 5.29 (d, J=8.5 Hz, 1H), 5.25-5.15 (m, 1H), 5.11 (q, J 7.0 Hz, 1H), 2.55-2.36 (m, 4H), 2.34-2.21 (m, 1H), 2.17-1.95 (m, 3H), 1.92-1.66 (m, 5H), 1.62-1.31 (m, 6H). MS (ESI) m/z: [M+H]⁺ Found 606.3.

### Example 86

### 1-((2,2-Difluorocyclobutyl)methyl)-N—((S)-(4,4-difluorocyclohexyl)(5-((R)-1-(4,4,4-trifluorobutanamido)ethyl)-1H-benzo[d]imidazol-2-yl)methyl)-1H-1,2,3-triazole-5-carboxamide

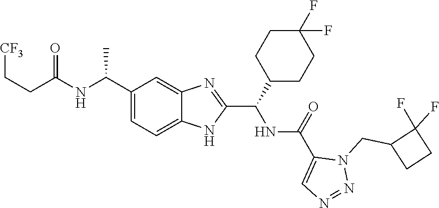 [SMILES: C[C@@H](NC(=O)CCC(F)(F)F)c1ccc2[nH]c([C@@H](NC(=O)c3cnnn3CC3CCC3(F)F)C3CCC(F)(F)CC3)nc2c1]

The title compound was prepared as described in the synthesis of Example 78, using 1-((2,2-difluorocyclobutyl)methyl)-1H-1,2,3-triazole-5-carboxylic acid (Intermediate 108) in place of 2-((3-cyanobicyclo[1.1.1]pentan-1-yl)methyl)-2H-1,2,3-triazole-4-carboxylic acid, and purified via silica gel chromatography (0-100% (10% MeOH in EtOAc)/DCM) to provide the title compound as a white solid. ¹H NMR (500 MHz, CD₃OD) δ 8.27 (d, J=6.2 Hz, 1H), 7.68-7.31 (m, 2H), 7.25 (dd, J=8.4, 1.7 Hz, 1H), 5.19 (dd, J=8.7, 5.3 Hz, 1H), 5.11 (q, J=7.0 Hz, 1H), 5.05-4.89 (m, 1H), 4.88-4.73 (m, 1H), 3.43-3.33 (m, 1H), 2.56-2.35 (m, 4H), 2.35-2.21 (m, 1H), 2.18-1.96 (m, 3H), 1.92-1.69 (m, 2H), 1.68-1.53 (m, 3H), 1.53-1.45 (m, 3H), 1.45-1.33 (m, 4H). MS (ESI) m/z: [M+H]⁺ Found 632.2.

### Example 87

### 2-((2,2-Difluorocyclobutyl)methyl)-N—((S)-(4,4-difluorocyclohexyl)(5-((R)-1-(4,4,4-trifluorobutanamido)ethyl)-1H-benzo[d]imidazol-2-yl)methyl)-2H-1,2,3-triazole-4-carboxamide

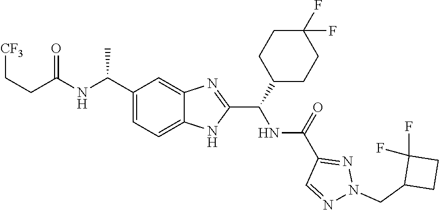 [SMILES: C[C@@H](NC(=O)CCC(F)(F)F)c1ccc2[nH]c([C@@H](NC(=O)c3cnn(CC4CCC4(F)F)n3)C3CCC(F)(F)CC3)nc2c1]

The title compound was prepared as described in the synthesis of Example 78, using 2-((2,2-difluorocyclobutyl)methyl)-2H-1,2,3-triazole-4-carboxylic acid (Intermediate 110) in place of 2-((3-cyanobicyclo[1.1.1]pentan-1-yl)methyl)-2H-1,2,3-triazole-4-carboxylic acid, and subjected to silica gel chromatography twice: 1^{st} chromatographic conditions (0-100% (10% MeOH in EtOAc)/DCM) 2^{nd} chromatographic conditions (0-100% EtOAc/DCM). Additional purification via preparative HPLC (Boston Prime, C18, 250×50 mm, 5 μM, 10-100% MeCN/water with 20 mM NH₃) provided the title compound as a white solid. ¹H NMR (500 MHz, CD₃OD) δ 8.07 (s, 1H), 7.58 (s, 1H), 7.44 (s, 1H), 7.24 (dd, J=8.5, 1.7 Hz, 1H), 5.26 (dd, J=8.7, 1.0 Hz, 1H), 5.11 (q, J 7.0 Hz, 1H), 4.78 (dd, J=14.0, 7.6 Hz, 1H), 4.64-4.55 (m, 1H), 3.60-3.42 (m, 1H), 2.63-2.37 (m, 5H), 2.33-2.21 (m, 1H), 2.17-1.92 (m, 4H), 1.91-1.62 (m, 2H), 1.60-1.43 (m, 5H), 1.43-1.32 (m, 1H). MS (ESI) m/z: [M+H]⁺ Found 632.2.

### Example 88

### 3-Cyano-1-(cyclobutylmethyl)-N—((S)-(4,4-difluorocyclohexyl)(5-((R)-1-(4,4,4-trifluorobutanamido)ethyl)-1H-benzo[d]imidazol-2-yl)methyl)-1H-pyrazole-4-carboxamide

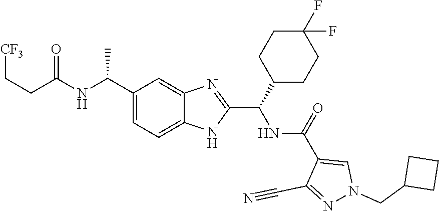 [SMILES: C[C@@H](NC(=O)CCC(F)(F)F)c1ccc2[nH]c([C@@H](NC(=O)c3cn(CC4CCC4)nc3C#N)C3CCC(F)(F)CC3)nc2c1]

The title compound was prepared as described in the synthesis of Example 80, using a 1:1 mixture of potassium 3-cyano-1-(cyclobutylmethyl)-1H-pyrazole-4-carboxylate and potassium 3-carbamoyl-1-(cyclobutylmethyl)-1H-pyrazole-4-carboxylate (Intermediate 123) in place of 1-((2,2-difluorocyclopropyl)methyl)-1H-1,2,3-triazole-5-carboxylic acid, and was further purified by preparative HPLC (Boston Prime, C18, 250×50 mm, 5 μM, 10-100% MeCN/water with 20 mM NH₃) to provide the title compound as a white solid. ¹H NMR (500 MHz, CD₃OD) δ 8.33 (s, 1H), 7.57-7.44 (m, 2H), 7.24 (dd, J=8.5, 1.7 Hz, 1H), 5.20 (d, J=8.5 Hz, 1H), 5.11 (q, J=7.0 Hz, 1H), 4.25 (d, J=7.4 Hz, 2H), 2.93-2.77 (m, 1H), 2.57-2.37 (m, 4H), 2.33-2.20 (m, 1H), 2.18-1.67 (m, 12H), 1.65-1.51 (m, 1H), 1.50 (d, J=7.0 Hz, 3H), 1.46-1.33 (m, 1H). MS (ESI) m/z: [M+H]⁺ Found 620.3.

### Example 89

### 1-(Cyclobutylmethyl)-N⁴—((S)-(4,4-difluorocyclohexyl)(5-((R)-1-(4,4,4-trifluorobutanamido)ethyl)-1H-benzo[d]imidazol-2-yl)methyl)-1H-pyrazole-3,4-dicarboxamide

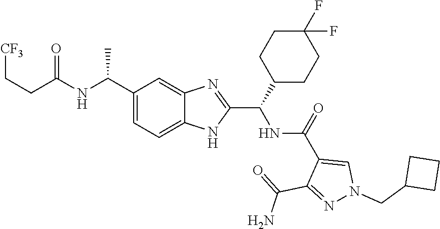 [SMILES: C[C@@H](NC(=O)CCC(F)(F)F)c1ccc2[nH]c([C@@H](NC(=O)c3cn(CC4CCC4)nc3C(N)=O)C3CCC(F)(F)CC3)nc2c1]

The title compound was prepared as described in the synthesis of Example 80, using a 1:1 mixture of potassium 3-cyano-1-(cyclobutylmethyl)-1H-pyrazole-4-carboxylate and potassium 3-carbamoyl-1-(cyclobutylmethyl)-1H-pyrazole-4-carboxylate (Intermediate 123) in place of 1-((2,2-difluorocyclopropyl)methyl)-1H-1,2,3-triazole-5-carboxylic acid, and was further purified by preparative HPLC (Boston Prime, C18, 250×50 mm, 5 mM, 10-100% MeCN/water with 20 mM NH₃) to provide the title compound as a white solid. ¹H NMR (500 MHz, CD₃OD) δ 8.14 (s, 1H), 7.56-7.38 (m, 2H), 7.20 (dd, J=8.5, 1.7 Hz, 1H), 5.19 (d, J=7.0 Hz, 1H), 5.10 (q, J=7.0 Hz, 1H), 4.21 (d, J=7.4 Hz, 2H), 2.92-2.81 (m, 1H), 2.55-2.39 (m, 4H), 2.35-2.24 (m, 1H), 2.13-1.99 (m, 4H), 1.99-1.72 (m, 7H), 1.71-1.56 (m, 2H), 1.53-1.41 (m, 4H). MS (ESI) m/z: [M+H]⁺ Found 638.3.

### Example 90

### 4-Cyano-1-(cyclobutylmethyl)-N—((S)-(4,4-difluorocyclohexyl)(5-((R)-1-(4,4,4-trifluorobutanamido)ethyl)-1H-benzo[d]imidazol-2-yl)methyl)-1H-pyrazole-5-carboxamide

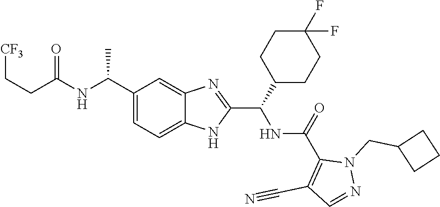 [SMILES: C[C@@H](NC(=O)CCC(F)(F)F)c1ccc2[nH]c([C@@H](NC(=O)c3c(C#N)cnn3CC3CCC3)C3CCC(F)(F)CC3)nc2c1]

The title compound was prepared as described in the synthesis of Example 78, using potassium 4-cyano-1-(cyclobutylmethyl)-1H-pyrazole-5-carboxylate (Intermediate 127) in place of 2-((3-cyanobicyclo[1.1.1]pentan-1-yl)methyl)-2H-1,2,3-triazole-4-carboxylic acid, and purified via silica gel chromatography (0-100% (10% MeOH in EtOAc)/DCM) to provide the title compound as a white solid. ¹H NMR (500 MHz, CDCl₃) δ 11.02-10.25 (m, 1H), 7.47-7.29 (m, 1H), 7.10 (s, 1H), 6.91-6.70 (m, 1H), 6.70-6.59 (m, 1H), 5.93 (d, J=7.7 Hz, 1H), 4.85-4.73 (m, 1H), 4.71-4.57 (m, 1H), 4.02-3.83 (m, 2H), 2.41-2.03 (m, 1H), 2.02-1.72 (m, 5H), 1.67-1.46 (m, 3H), 1.46-1.34 (m, 2H), 1.34-1.07 (m, 8H), 1.07-0.82 (m, 5H). MS (ESI) m/z: [M+H]⁺ Found 620.3.

### Example 91

### 4-Cyano-1-(cyclobutylmethyl)-N—((S)-(4,4-difluorocyclohexyl)(5-((R)-1-(4,4,4-trifluorobutanamido)ethyl)-1H-benzo[d]imidazol-2-yl)methyl)-1H-pyrazole-3-carboxamide

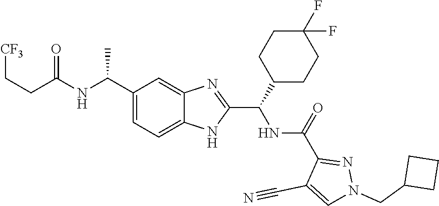 [SMILES: C[C@@H](NC(=O)CCC(F)(F)F)c1ccc2[nH]c([C@@H](NC(=O)c3nn(CC4CCC4)cc3C#N)C3CCC(F)(F)CC3)nc2c1]

The title compound was prepared as described in the synthesis of Example 78, using 4-cyano-1-(cyclobutylmethyl)-1H-pyrazole-3-carboxylic acid (Intermediate 129) in place of 2-((3-cyanobicyclo[1.1.1]pentan-1-yl)methyl)-2H-1,2,3-triazole-4-carboxylic acid, and purified via silica gel chromatography (0-100% (10% MeOH in EtOAc)/DCM) to provide the title compound as a white solid. ¹H NMR (500 MHz, CD₃OD) δ 8.35 (s, 1H), 7.67-7.30 (m, 2H), 7.24 (dd, J=8.4, 1.7 Hz, 1H), 5.24 (d, J=8.6 Hz, 1H), 5.11 (q, J=7.0 Hz, 1H), 4.26 (d, J=7.5 Hz, 2H), 2.95-2.83 (m, 1H), 2.56-2.38 (m, 4H), 2.33-2.21 (m, 1H), 2.16-1.67 (m, 11H), 1.60-1.44 (m, 5H), 1.44-1.33 (m, 1H). MS (ESI) m/z: [M+H]⁺ Found 620.2.

### Example 92

### N—((S)-(4,4-Difluorocyclohexyl)(5-((R)-1-(4,4,4-trifluorobutanamido)ethyl)-1H-benzo[d]imidazol-2-yl)methyl)-1-ethyl-5-(trifluoromethyl)-1H-pyrazole-3-carboxamide

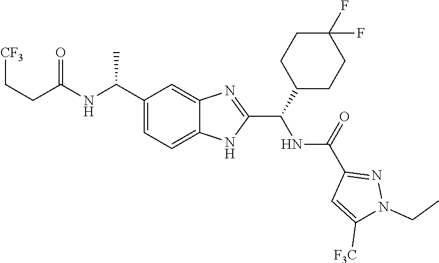 [SMILES: CCn1nc(C(=O)N[C@H](c2nc3cc([C@@H](C)NC(=O)CCC(F)(F)F)ccc3[nH]2)C2CCC(F)(F)CC2)cc1C(F)(F)F]

The title compound was prepared as described in the synthesis of Example 78, using 1-ethyl-5-(trifluoromethyl)-1H-pyrazole-3-carboxylic acid in place of 2-((3-cyanobicyclo[1.1.1]pentan-1-yl)methyl)-2H-1,2,3-triazole-4-carboxylic acid, and purified via silica gel chromatography (0-100% (10% MeOH in EtOAc)/DCM) to provide the title compound as a white solid. ¹H NMR (400 MHz, CD₃OD) δ 8.57 (d, J=7.8 Hz, 1H), 7.57-7.45 (m, 2H), 7.25 (dd, J=8.5, 1.6 Hz, 1H), 7.16 (d, J=0.7 Hz, 1H), 5.26 (d, J=8.7 Hz, 1H), 5.18-5.04 (m, 1H), 4.38 (q, J=7.1 Hz, 2H), 2.56-2.38 (m, 4H), 2.33-2.20 (m, 1H), 2.17-1.95 (m, 3H), 1.92-1.65 (m, 2H), 1.60-1.45 (m, 7H), 1.45-1.31 (m, 1H). MS (ESI) m/z: [M+H]⁺ Found 623.3.

### Example 93

### N—((S)-(4,4-Difluorocyclohexyl)(5-((R)-1-(4,4,4-trifluorobutanamido)ethyl)-1H-benzo[d]imidazol-2-yl)methyl)-5-hydroxy-1-(3,3,3-trifluoropropyl)-1H-pyrazole-3-carboxamide

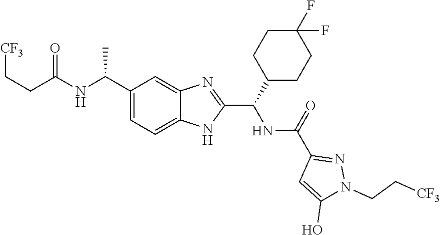 [SMILES: C[C@@H](NC(=O)CCC(F)(F)F)c1ccc2[nH]c([C@@H](NC(=O)c3cc(O)n(CCC(F)(F)F)n3)C3CCC(F)(F)CC3)nc2c1]

The title compound was prepared as described in the synthesis of Example 78, using potassium 5-hydroxy-1-(3,3,3-trifluoropropyl)-1H-pyrazole-3-carboxylate (Intermediate 125) in place of 2-((3-cyanobicyclo[1.1.1]pentan-1-yl)methyl)-2H-1,2,3-triazole-4-carboxylic acid, and purified via silica gel chromatography (0-100% (10% MeOH in EtOAc)/DCM) followed by preparative HPLC (Boston Prime, C18, 250×50 mm, 5 μM, 10-100% MeCN/water with 20 mM NH₃) to provide the title compound as a white solid. ¹H NMR (500 MHz, CD₃OD) δ 7.64-7.35 (m, 2H), 7.24 (dd, J=8.4, 1.6 Hz, 1H), 6.33 (s, 1H), 5.20 (d, J=8.6 Hz, 1H), 5.11 (q, J=6.9 Hz, 1H), 4.36 (t, J=6.1 Hz, 2H), 2.74-2.61 (m, 2H), 2.55-2.38 (m, 4H), 2.32-2.18 (m, 1H), 2.16-1.95 (m, 3H), 1.90-1.66 (m, 2H), 1.62-1.54 (m, 1H), 1.54-1.43 (m, 4H), 1.43-1.32 (m, 1H). MS (ESI) m/z: [M+H]⁺ Found 639.3.

### Example 94

### N—((S)-(4,4-Difluorocyclohexyl)(5-((R)-1-(4,4,4-trifluorobutanamido)ethyl)-1H-benzo[d]imidazol-2-yl)methyl)cyclopropanecarboxamide

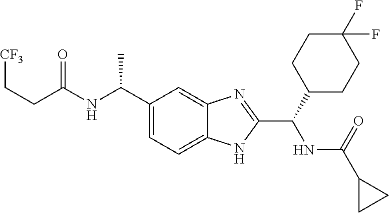 [SMILES: C[C@@H](NC(=O)CCC(F)(F)F)c1ccc2[nH]c([C@@H](NC(=O)C3CC3)C3CCC(F)(F)CC3)nc2c1]

The title compound was prepared as described in the synthesis of Example 78, using cyclopropanecarboxylic acid in place of 2-((3-cyanobicyclo[1.1.1]pentan-1-yl)methyl)-2H-1,2,3-triazole-4-carboxylic acid, and purified via silica gel chromatography (0-100% EtOAc/DCM) to provide the title compound as a white solid. ¹H NMR (500 MHz, CD₃OD) δ 7.55-7.45 (m, 2H), 7.22 (dd, J=8.4, 1.7 Hz, 1H), 5.11 (q, J=6.9 Hz, 1H), 5.06 (d, J=8.2 Hz, 1H), 2.57-2.37 (m, 4H), 2.23-2.13 (m, 1H), 2.13-1.89 (m, 3H), 1.87-1.66 (m, 4H), 1.60-1.41 (m, 4H), 1.40-1.30 (m, 1H), 0.93-0.70 (m, 4H). MS (ESI) m/z: [M+H]⁺ Found 501.2.

### Example 95

### N—((S)-(4,4-Difluorocyclohexyl)(5-((R)-1-(4,4,4-trifluorobutanamido)ethyl)-1H-benzo[d]imidazol-2-yl)methyl)-1-methylcyclopropane-1-carboxamide

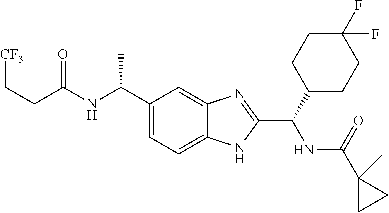 [SMILES: C[C@@H](NC(=O)CCC(F)(F)F)c1ccc2[nH]c([C@@H](NC(=O)C3(C)CC3)C3CCC(F)(F)CC3)nc2c1]

The title compound was prepared as described in the synthesis of Example 78, using 1-methylcyclopropane-1-carboxylic acid in place of 2-((3-cyanobicyclo[1.1.1]pentan-1-yl)methyl)-2H-1,2,3-triazole-4-carboxylic acid, and purified via silica gel chromatography (0-100% EtOAc/DCM) followed by preparative HPLC (Boston Prime, C18, 250×50 mm, 5 μM, 10-100% MeCN/water with 20 mM NH₃) to provide the title compound as a white solid. ¹H NMR (500 MHz, CD₃OD) δ 7.67-7.32 (m, 2H), 7.24 (dd, J=8.4, 1.7 Hz, 1H), 5.11 (q, J=7.0 Hz, 1H), 5.06 (d, J=8.8 Hz, 1H), 2.58-2.36 (m, 4H), 2.22-2.04 (m, 2H), 2.04-1.91 (m, 2H), 1.88-1.64 (m, 2H), 1.54-1.43 (m, 4H), 1.39 (s, 5H), 1.19-1.12 (m, 1H), 1.09-1.03 (m, 1H), 0.70-0.57 (m, 2H). MS (ESI) m/z: [M+H]⁺ Found 515.2.

### Example 96

### N—((S)-(4,4-Difluorocyclohexyl)(5-((R)-1-(4,4,4-trifluorobutanamido)ethyl)-1H-benzo[d]imidazol-2-yl)methyl)-1-fluorocyclopropane-1-carboxamide

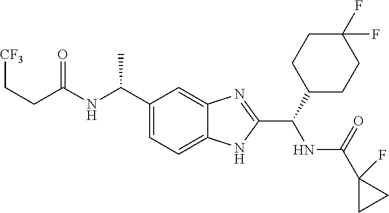 [SMILES: C[C@@H](NC(=O)CCC(F)(F)F)c1ccc2[nH]c([C@@H](NC(=O)C3(F)CC3)C3CCC(F)(F)CC3)nc2c1]

The title compound was prepared as described in the synthesis of Example 78, using 1-fluorocyclopropane-1-carboxylic acid in place of 2-((3-cyanobicyclo[1.1.1]pentan-1-yl)methyl)-2H-1,2,3-triazole-4-carboxylic acid, and purified via silica gel chromatography (0-100% EtOAc/DCM) followed by preparative HPLC (Boston Prime, C18, 250×50 mm, 5 μM, 10-100% MeCN/water with 20 mM NH₃) to provide the title compound as a white solid. ¹H NMR (500 MHz, CD₃OD) δ 7.66-7.34 (m, 2H), 7.24 (dd, J=8.4, 1.7 Hz, 1H), 5.19-5.05 (m, 2H), 2.55-2.38 (m, 4H), 2.27-2.15 (m, 1H), 2.15-2.05 (m, 1H), 2.05-1.94 (m, 2H), 1.90-1.65 (m, 2H), 1.55-1.46 (m, 4H), 1.46-1.41 (m, 1H), 1.41-1.19 (m, 5H). MS (ESI) m/z: [M+H]⁺ Found 519.1.

### Example 97

### N—((S)-(4,4-Difluorocyclohexyl)(5-((R)-1-(4,4,4-trifluorobutanamido)ethyl)-1H-benzo[d]imidazol-2-yl)methyl)-1-(trifluoromethyl)cyclopropane-1-carboxamide

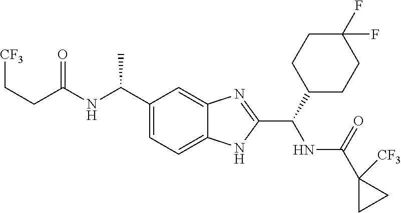 [SMILES: C[C@@H](NC(=O)CCC(F)(F)F)c1ccc2[nH]c([C@@H](NC(=O)C3(C(F)(F)F)CC3)C3CCC(F)(F)CC3)nc2c1]

The title compound was prepared as described in the synthesis of Example 78, using 1-(trifluoromethyl)cyclopropane-1-carboxylic acid in place of 2-((3-cyanobicyclo[1.1.1]pentan-1-yl)methyl)-2H-1,2,3-triazole-4-carboxylic acid to provide the title compound as a white solid. ¹H NMR (500 MHz, CD₃OD) δ 7.66-7.34 (m, 2H), 7.24 (dd, J=8.4, 1.7 Hz, 1H), 5.15-5.03 (m, 2H), 2.57-2.37 (m, 4H), 2.25-2.14 (m, 1H), 2.14-2.04 (m, 1H), 2.04-1.92 (m, 2H), 1.89-1.64 (m, 2H), 1.49 (d, J=7.0 Hz, 3H), 1.47-1.22 (m, 7H). MS (ESI) m/z: [M+H]⁺ Found 569.3.

### Example 98

### (1R,2S)—N—((S)-(4,4-Difluorocyclohexyl)(5-((R)-1-(4,4,4-trifluorobutanamido)ethyl)-1H-benzo[d]imidazol-2-yl)methyl)-2-fluorocyclopropane-1-carboxamide

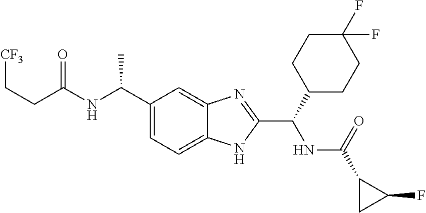 [SMILES: C[C@@H](NC(=O)CCC(F)(F)F)c1ccc2[nH]c([C@@H](NC(=O)[C@H]3C[C@@H]3F)C3CCC(F)(F)CC3)nc2c1]

The title compound was prepared as described in the synthesis of Example 80, using (1R,2S)-2-fluorocyclopropane-1-carboxylic acid in place of 1-((2,2-difluorocyclopropyl)methyl)-1H-1,2,3-triazole-5-carboxylic acid and purified via silica gel chromatography (0-100% (10% MeOH in EtOAc)/hexanes) to provide the title compound as a white solid. ¹H NMR (500 MHz, CD₃OD) δ 7.67-7.33 (m, 2H), 7.23 (dd, J=8.4, 1.7 Hz, 1H), 5.11 (q, J=7.0 Hz, 1H), 5.02 (d, J=7.9 Hz, 1H), 4.80-4.63 (m 1H), 2.57-2.38 (m, 4H), 2.28-2.13 (m, 2H), 2.13-1.96 (m, 2H), 1.96-1.87 (m, 1H), 1.87-1.67 (m, 2H), 1.60-1.52 (m, 1H), 1.52-1.46 (m, 4H), 1.46-1.28 (m, 2H), 1.28-1.18 (m, 1H). MS (ESI) m/z: [M+H]⁺ Found 519.2.

### Example 99

### (1R,2R)—N—((S)-(4,4-Difluorocyclohexyl)(5-((R)-1-(4,4,4-trifluorobutanamido)ethyl)-1H-benzo[d]imidazol-2-yl)methyl)-2-fluorocyclopropane-1-carboxamide

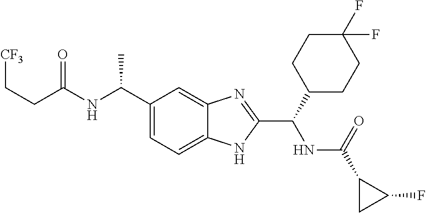 [SMILES: C[C@@H](NC(=O)CCC(F)(F)F)c1ccc2[nH]c([C@@H](NC(=O)[C@H]3C[C@H]3F)C3CCC(F)(F)CC3)nc2c1]

The title compound was prepared as described in the synthesis of Example 80, using (1R,2R)-2-fluorocyclopropane-1-carboxylic acid in place of 1-((2,2-difluorocyclopropyl)methyl)-1H-1,2,3-triazole-5-carboxylic acid and purified via silica gel chromatography (0-100% (10% MeOH in EtOAc)/hexanes) to provide the title compound as a white solid. ¹H NMR (500 MHz, CD₃OD) δ 7.67-7.34 (m, 2H), 7.22 (dd, J=8.4, 1.7 Hz, 1H), 5.18-5.02 (m, 2H), 4.83-4.64 (m, 1H), 2.56-2.38 (m, 4H), 2.24-2.13 (m, 1H), 2.13-2.05 (m, 1H), 2.05-1.93 (m, 2H), 1.93-1.87 (m, 1H), 1.87-1.60 (m, 3H), 1.58-1.41 (m, 5H), 1.41-1.27 (m, 1H), 1.16-1.06 (m, 1H). MS (ESI) m/z: [M+H]⁺ Found 519.2.

### Example 100

### (1S,2R)—N—((S)-(4,4-Difluorocyclohexyl)(5-((R)-1-(4,4,4-trifluorobutanamido)ethyl)-1H-benzo[d]imidazol-2-yl)methyl)-2-fluorocyclopropane-1-carboxamide

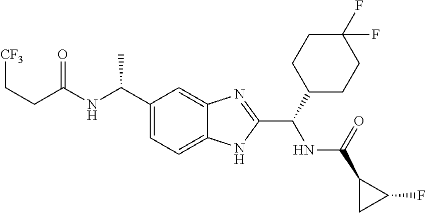 [SMILES: C[C@@H](NC(=O)CCC(F)(F)F)c1ccc2[nH]c([C@@H](NC(=O)[C@@H]3C[C@H]3F)C3CCC(F)(F)CC3)nc2c1]

The title compound was prepared as described in the synthesis of Example 80, using (1S,2R)-2-fluorocyclopropane-1-carboxylic acid in place of 1-((2,2-difluorocyclopropyl)methyl)-1H-1,2,3-triazole-5-carboxylic acid and purified via silica gel chromatography (0-100% (10% MeOH in EtOAc)/hexanes) to provide the title compound as a white solid. ¹H NMR (500 MHz, CD₃OD) δ 7.67-7.34 (m, 2H), 7.22 (dd, J=8.4, 1.7 Hz, 1H), 5.18-5.02 (m, 2H), 4.83-4.64 (m, 1H), 2.56-2.38 (m, 4H), 2.24-2.13 (m, 1H), 2.13-2.05 (m, 1H), 2.05-1.93 (m, 2H), 1.93-1.87 (m, 1H), 1.87-1.60 (m, 3H), 1.58-1.41 (m, 5H), 1.41-1.27 (m, 1H), 1.16-1.06 (m, 1H). MS (ESI) m/z: [M+H]⁺ Found 519.2.

### Example 101

### (1S,2S)—N—((S)-(4,4-Difluorocyclohexyl)(5-((R)-1-(4,4,4-trifluorobutanamido)ethyl)-1H-benzo[d]imidazol-2-yl)methyl)-2-fluorocyclopropane-1-carboxamide

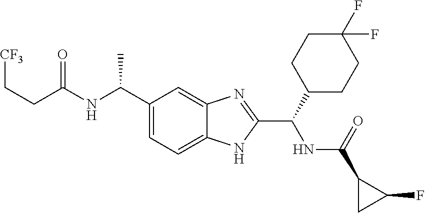 [SMILES: C[C@@H](NC(=O)CCC(F)(F)F)c1ccc2[nH]c([C@@H](NC(=O)[C@@H]3C[C@@H]3F)C3CCC(F)(F)CC3)nc2c1]

The title compound was prepared as described in the synthesis of Example 80, using (1S,2S)-2-fluorocyclopropane-1-carboxylic acid in place of 1-((2,2-difluorocyclopropyl)methyl)-1H-1,2,3-triazole-5-carboxylic acid and purified via silica gel chromatography (0-100% (10% MeOH in EtOAc)/hexanes) to provide the title compound as a white solid. ¹H NMR (500 MHz, CD₃OD) δ 7.62-7.35 (m, 2H), 7.23 (dd, J=8.5, 1.7 Hz, 1H), 5.17-5.06 (m, 2H), 4.87-4.68 (m, 1H), 2.57-2.37 (m, 4H), 2.25-2.13 (m, 1H), 2.13-1.97 (m, 2H), 1.97-1.87 (m, 2H), 1.87-1.69 (m, 2H), 1.68-1.59 (m, 1H), 1.58-1.52 (m, 1H), 1.52-1.42 (m, 4H), 1.42-1.32 (m, 1H), 1.12-1.01 (m, 1H). MS (ESI) m/z: [M+H]⁺ Found 519.2.

### Example 102

### cis-(1,2)-2-Cyano-N—((S)-(4,4-difluorocyclohexyl)(5-((R)-1-(4,4,4-trifluorobutanamido)ethyl)-1H-benzo[d]imidazol-2-yl)methyl)cyclopropane-1-carboxamide

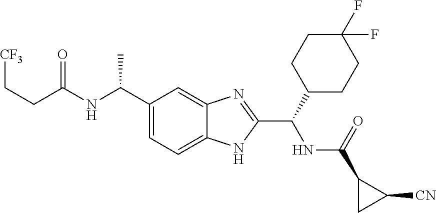 [SMILES: C[C@@H](NC(=O)CCC(F)(F)F)c1ccc2[nH]c([C@@H](NC(=O)[C@@H]3C[C@@H]3C#N)C3CCC(F)(F)CC3)nc2c1]

The mixture of title compounds was prepared as described in the synthesis of Example 80, using cis-2-cyanocyclopropane-1-carboxylic acid in place of 1-((2,2-difluorocyclopropyl)methyl)-1H-1,2,3-triazole-5-carboxylic acid and purified via silica gel chromatography (0-100% (10% MeOH in EtOAc)/hexanes) to provide the title compound, a 1:1 mixture of cis-isomers, as a white solid. ¹H NMR (500 MHz, CD₃OD) δ 7.66-7.34 (m, 2H), 7.27-7.18 (m, 1H), 5.18-5.05 (m, 2H), 2.56-2.37 (m, 4H), 2.34-2.25 (m, 1H), 2.25-2.16 (m, 1H), 2.15-2.05 (m, 1H), 2.05-1.90 (m, 3H), 1.89-1.67 (m, 2H), 1.63-1.43 (m, 6H), 1.43-1.36 (m, 1H), 1.36-1.26 (m, 1H).

### Example 103

### trans-(1S*,2S*)-2-Cyano-N—((S)-(4,4-difluorocyclohexyl)(5-((R)-1-(4,4,4-trifluorobutanamido)ethyl)-1H-benzo[d]imidazol-2-yl)methyl)cyclopropane-1-carboxamide

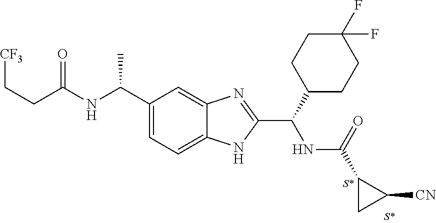 [SMILES: C[C@@H](NC(=O)CCC(F)(F)F)c1ccc2[nH]c([C@@H](NC(=O)[C@H]3C[C@@H]3C#N)C3CCC(F)(F)CC3)nc2c1]

### Example 104

### trans-(1R*,2R*)-2-Cyano-N—((S)-(4,4-difluorocyclohexyl)(5-((R)-1-(4,4,4-trifluorobutanamido)ethyl)-1H-benzo[d]imidazol-2-yl)methyl)cyclopropane-1-carboxamide

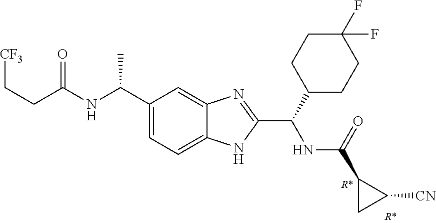 [SMILES: C[C@@H](NC(=O)CCC(F)(F)F)c1ccc2[nH]c([C@@H](NC(=O)[C@@H]3C[C@H]3C#N)C3CCC(F)(F)CC3)nc2c1]

The title compounds were prepared as described for the synthesis of Example 80, using trans-2-cyanocyclopropane-1-carboxylic acid in place of 1-((2,2-difluorocyclopropyl)methyl)-1H-1,2,3-triazole-5-carboxylic acid to provide the title compounds as a mixture of diastereomers. The diastereomers were separated by silica gel chromatography (0-100% EtOAc/hexanes) to afford the title compounds as single trans-diastereomers. Example 103 was the first eluting isomer, isolated as a white solid. Example 104 was the second eluting isomer, isolated as a white solid. Example 103: ¹H NMR (500 MHz, CD₃OD) δ 7.70-7.31 (m, 2H), 7.23 (dd, J=8.4, 1.6 Hz, 1H), 5.11 (q, J=7.0 Hz, 1H), 5.05 (d, J=7.6 Hz, 1H), 2.57-2.38 (m, 4H), 2.26-2.16 (m, 1H), 2.14-1.96 (m, 2H), 1.95-1.85 (m, 2H), 1.85-1.68 (m, 2H), 1.63-1.53 (m, 1H), 1.53-1.42 (m, 6H), 1.42-1.25 (m, 2H). MS (ESI) m/z: [M+H]⁺ Found 526.2. Example 104: ¹H NMR (500 MHz, CD₃OD) δ 7.67-7.32 (m, 2H), 7.23 (dd, J=8.4, 1.7 Hz, 1H), 5.11 (q, J=7.0 Hz, 1H), 5.02 (d, J=8.1 Hz, 1H), 2.56-2.38 (m, 4H), 2.25-2.15 (m, 1H), 2.15-2.07 (m, 1H), 2.07-1.99 (m, 1H), 1.99-1.92 (m, 2H), 1.89-1.68 (m, 2H), 1.61-1.43 (m, 6H), 1.43-1.26 (m, 3H). MS (ESI) m/z: [M+H]⁺ Found 526.2.

### Example 105

### trans-(1S*,2S*)—N—((S)-(4,4-Difluorocyclohexyl)(5-((R)-1-(4,4,4-trifluorobutanamido)ethyl)-1H-benzo[d]imidazol-2-yl)methyl)-2-(trifluoromethyl)cyclopropane-1-carboxamide

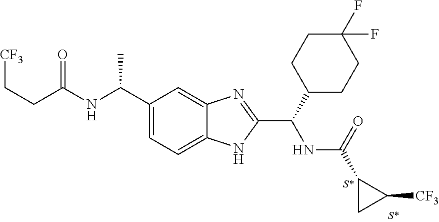 [SMILES: C[C@@H](NC(=O)CCC(F)(F)F)c1ccc2[nH]c([C@@H](NC(=O)[C@H]3C[C@@H]3C(F)(F)F)C3CCC(F)(F)CC3)nc2c1]

### Example 106

### trans-(1R*,2R*)—N—((S)-(4,4-Difluorocyclohexyl)(5-((R)-1-(4,4,4-trifluorobutanamido)ethyl)-1H-benzo[d]imidazol-2-yl)methyl)-2-(trifluoromethyl)cyclopropane-1-carboxamide

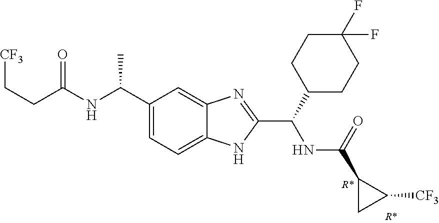 [SMILES: C[C@@H](NC(=O)CCC(F)(F)F)c1ccc2[nH]c([C@@H](NC(=O)[C@@H]3C[C@H]3C(F)(F)F)C3CCC(F)(F)CC3)nc2c1]

The title compounds were prepared as described for the synthesis of Example 80, using trans-(2-trifluoromethyl)cyclopropane-1-carboxylic acid in place of 1-((2,2-difluorocyclopropyl)methyl)-1H-1,2,3-triazole-5-carboxylic acid to provide the title compounds as a mixture of diastereomers. The diastereomers were separated by silica gel chromatography twice (0-100% EtOAc/hexanes) followed by (0-70% EtOAc/hexanes) to afford the title compounds as single trans-diastereomers. Example 105 was the first eluting isomer, isolated as a white solid. Example 106 was the second eluting isomer, isolated as a white solid. Example 105: ¹H NMR (500 MHz, CD₃OD) δ 7.72-7.31 (m, 2H), 7.23 (dd, J=8.4, 1.7 Hz, 1H), 5.11 (q, J=7.0 Hz, 1H), 5.03 (d, J=8.1 Hz, 1H), 2.56-2.37 (m, 4H), 2.26-2.15 (m, 2H), 2.15-2.06 (m, 2H), 2.06-1.98 (m, 1H), 1.98-1.90 (m, 1H), 1.89-1.68 (m, 2H), 1.60-1.51 (m, 1H), 1.51-1.45 (m, 4H), 1.41-1.30 (m, 1H), 1.28-1.22 (m, 1H), 1.22-1.16 (m, 1H). MS (ESI) m/z: [M+H]⁺ Found 569.3. Example 106: ¹H NMR (500 MHz, CD₃OD) δ 7.70-7.34 (m, 2H), 7.23 (dd, J=8.4, 1.7 Hz, 1H), 5.11 (q, J=7.0 Hz, 1H), 5.03 (d, J=8.2 Hz, 1H), 2.57-2.36 (m, 4H), 2.26-2.14 (m, 2H), 2.14-1.89 (m, 4H), 1.89-1.67 (m, 2H), 1.59-1.41 (m, 4H), 1.41-1.18 (m, 4H). MS (ESI) m/z: [M+H]⁺ Found 569.3.

### Example 107

### N—((R)-1-(2-((S)-(4,4-Difluorocyclohexyl)(2-((R*)-2,2-difluorocyclopropyl)acetamido)methyl)-1H-benzo[d]imidazol-5-yl)ethyl)-4,4,4-trifluorobutanamide

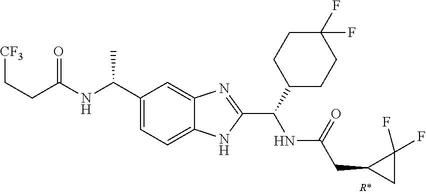 [SMILES: C[C@@H](NC(=O)CCC(F)(F)F)c1ccc2[nH]c([C@@H](NC(=O)C[C@@H]3CC3(F)F)C3CCC(F)(F)CC3)nc2c1]

### Example 108

### N—((R)-1-(2-((S)-(4,4-Difluorocyclohexyl)(2-((S*)-2,2-difluorocyclopropyl)acetamido)methyl)-1H-benzo[d]imidazol-5-yl)ethyl)-4,4,4-trifluorobutanamide

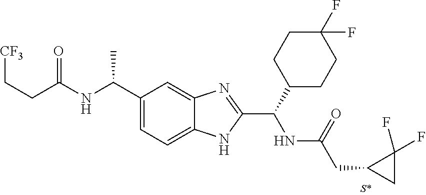 [SMILES: C[C@@H](NC(=O)CCC(F)(F)F)c1ccc2[nH]c([C@@H](NC(=O)C[C@H]3CC3(F)F)C3CCC(F)(F)CC3)nc2c1]

The title compounds were prepared as described for the synthesis of Example 80, using 2-(2,2-difluorocyclopropane)acetic acid in place of 1-((2,2-difluorocyclopropyl)methyl)-1H-1,2,3-triazole-5-carboxylic acid to provide the title compounds as a mixture of diastereomers. The diastereomers were separated by silica gel chromatography (0-100% EtOAc/hexanes) to afford the title compounds as single diastereomers. Example 107 was the first eluting isomer, isolated as a white solid. Example 108 was the second eluting isomer, isolated as a white solid. Example 107: ¹H NMR (500 MHz, CD₃OD) δ 7.63-7.36 (m, 2H), 7.23 (dd, J=8.4, 1.7 Hz, 1H), 5.11 (q, J 7.0 Hz, 1H), 5.07 (d, J=8.1 Hz, 1H), 2.60-2.35 (m, 6H), 2.24-2.14 (m, 1H), 2.13-1.96 (m, 2H), 1.96-1.67 (m, 4H), 1.60-1.43 (m, 6H), 1.43-1.28 (m, 1H), 1.16-1.06 (m, 1H). MS (ESI) m/z: [M+H]⁺ Found 551.3. Example 108: ¹H NMR (500 MHz, CD₃OD) δ 7.62-7.35 (m, 2H), 7.23 (dd, J=8.5, 1.6 Hz, 1H), 5.11 (q, J=7.0 Hz, 1H), 5.06 (d, J=8.1 Hz, 1H), 2.57-2.38 (m, 6H), 2.23-2.13 (m, 1H), 2.13-1.97 (m, 2H), 1.97-1.67 (m, 5H), 1.60-1.44 (m, 5H), 1.44-1.27 (m, 1H), 1.17-1.05 (m, 1H).

### Example 109

### N—((S)-(4,4-Difluorocyclohexyl)(5-((R*)-1-(4,4,4-trifluorobutanamido)ethyl)-1H-benzo[d]imidazol-2-yl)methyl)-2,2-difluoro-1-methylcyclopropane-1-carboxamide

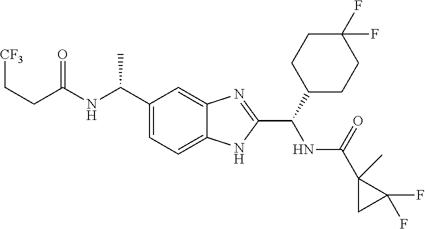 [SMILES: C[C@@H](NC(=O)CCC(F)(F)F)c1ccc2[nH]c([C@@H](NC(=O)C3(C)CC3(F)F)C3CCC(F)(F)CC3)nc2c1]

The title compound was prepared as described in the synthesis of Example 78, using 2,2-difluoro-1-methyl-cyclopropane carboxylic acid in place of 2-((3-cyanobicyclo[1.1.1]pentan-1-yl)methyl)-2H-1,2,3-triazole-4-carboxylic acid, and using 0-100% EtOAc/DCM instead of 0-100% EtOAc/hexanes for silica gel chromatography followed by basic preparative HPLC to provide the title compound as a white solid. ¹H NMR (500 MHz, CD₃OD) δ 7.56-7.41 (m, 2H), 7.24 (dd, J=8.4, 1.6 Hz, 1H), 5.11 (q, J=7.0 Hz, 1H), 5.07-4.99 (m, 1H), 2.57-2.37 (m, 4H), 2.25-2.13 (m, 1H), 2.13-2.04 (m, 1H), 2.04-1.92 (m, 2H), 1.89-1.63 (m, 2H), 1.55-1.19 (m, 11H). MS (ESI) m/z: [M+H]⁺ Found 551.3.

### Example 110

### N—((S)-(4,4-Difluorocyclohexyl)(5-((R)-1-(4,4,4-trifluorobutanamido)ethyl)-1H-benzo[d]imidazol-2-yl)methyl)spiro[2.2]pentane-1-carboxamide

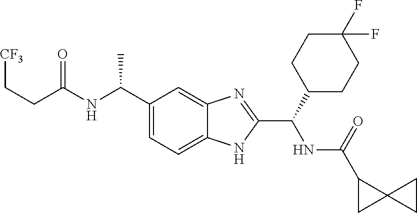 [SMILES: C[C@@H](NC(=O)CCC(F)(F)F)c1ccc2[nH]c([C@@H](NC(=O)C3CC34CC4)C3CCC(F)(F)CC3)nc2c1]

The title compound was prepared as described in the synthesis of Example 78, using spiro[2.2]pentane-1-carboxylic acid in place of 2-((3-cyanobicyclo[1.1.1]pentan-1-yl)methyl)-2H-1,2,3-triazole-4-carboxylic acid to provide the title compound as a white solid. ¹H NMR (500 MHz, CD₃OD) δ 7.54-7.44 (m, 2H), 7.26-7.18 (m, 1H), 5.15-5.01 (m, 2H), 2.53-2.40 (m, 4H), 2.20-1.91 (m, 4H), 1.91-1.65 (m, 2H), 1.57-1.46 (m, 4H), 1.46-1.40 (m, 2H), 1.40-1.25 (m, 2H), 0.98-0.64 (m, 5H). MS (ESI) m/z: [M+H]⁺ Found 527.3.

### Example 111

### N—((S)-(4,4-Difluorocyclohexyl)(5-((R)-1-(4,4,4-trifluorobutanamido)ethyl)-1H-benzo[d]imidazol-2-yl)methyl)spiro[2.3]hexane-1-carboxamide

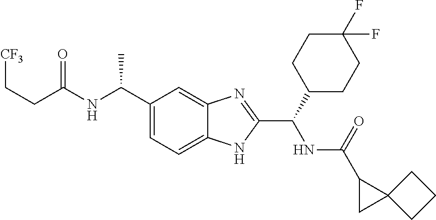 [SMILES: C[C@@H](NC(=O)CCC(F)(F)F)c1ccc2[nH]c([C@@H](NC(=O)C3CC34CCC4)C3CCC(F)(F)CC3)nc2c1]

The title compound was prepared as described in the synthesis of Example 78, using spiro[2.3]hexane-1-carboxylic acid in place of 2-((3-cyanobicyclo[1.1.1]pentan-1-yl)methyl)-2H-1,2,3-triazole-4-carboxylic acid, and purified via silica gel chromatography (0-100% (10% MeOH in EtOAc)/hexanes) to provide the title compound as a white solid. ¹H NMR (500 MHz, CD₃OD) δ 7.66-7.31 (m, 2H), 7.23 (dd, J=8.5, 1.7 Hz, 1H), 5.11 (q, J=7.0 Hz, 1H), 5.01 (d, J=9.0 Hz, 1H), 2.57-2.36 (m, 4H), 2.24-2.13 (m, 1H), 2.13-2.03 (m, 7H), 2.03-1.88 (m, 2H), 1.87-1.62 (m, 2H), 1.54-1.44 (m, 4H), 1.44-1.22 (m, 3H), 1.16-1.08 (m, 1H), 0.98-0.87 (m, 1H). MS (ESI) m/z: [M+H]⁺ Found 541.2.

### Example 112

### N—((S)-(4,4-Difluorocyclohexyl)(5-((R)-1-(4,4,4-trifluorobutanamido)ethyl)-1H-benzo[d]imidazol-2-yl)methyl)bicyclo[1.1.1]pentane-1-carboxamide

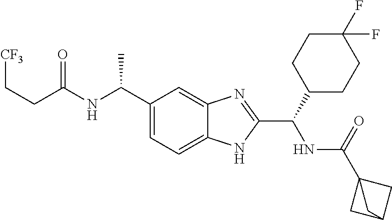 [SMILES: C[C@@H](NC(=O)CCC(F)(F)F)c1ccc2[nH]c([C@@H](NC(=O)C34CC(C3)C4)C3CCC(F)(F)CC3)nc2c1]

The title compound was prepared as described in the synthesis of Example 78, using bicyclo[1.1.1]pentane-1-carboxylic acid in place of 2-((3-cyanobicyclo[1.1.1]pentan-1-yl)methyl)-2H-1,2,3-triazole-4-carboxylic acid, and purified via silica gel chromatography (0-100% (10% MeOH in EtOAc)/hexanes) to provide the title compound as a white solid. ¹H NMR (500 MHz, CD₃OD) δ 7.66-7.31 (m, 2H), 7.23 (dd, J=8.5, 1.7 Hz, 1H), 5.11 (q, J=7.0 Hz, 1H), 5.01 (d, J=9.0 Hz, 1H), 2.57-2.36 (m, 4H), 2.24-2.13 (m, 1H), 2.13-2.03 (m, 7H), 2.03-1.88 (m, 2H), 1.87-1.62 (m, 2H), 1.54-1.44 (m, 4H), 1.44-1.22 (m, 3H). MS (ESI) m/z: [M+H]⁺ Found 527.1.

### Example 113

### cis-(1,2)-N—((S)-(4,4-Difluorocyclohexyl)(5-((R)-1-(4,4,4-trifluorobutanamido)ethyl)-1H-benzo[d]imidazol-2-yl)methyl)-2-(fluoromethyl)cyclobutane-1-carboxamide

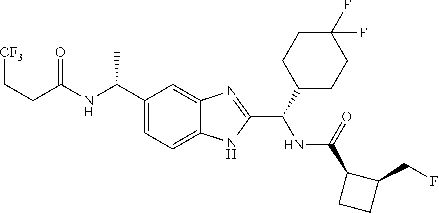 [SMILES: C[C@@H](NC(=O)CCC(F)(F)F)c1ccc2[nH]c([C@@H](NC(=O)[C@@H]3CC[C@@H]3CF)C3CCC(F)(F)CC3)nc2c1]

### Example 114

### trans-(1,2)-N—((S)-(4,4-Difluorocyclohexyl)(5-((R)-1-(4,4,4-trifluorobutanamido)ethyl)-1H-benzo[d]imidazol-2-yl)methyl)-2-(fluoromethyl)cyclobutane-1-carboxamide

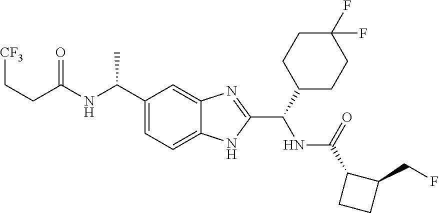 [SMILES: C[C@@H](NC(=O)CCC(F)(F)F)c1ccc2[nH]c([C@@H](NC(=O)[C@H]3CC[C@@H]3CF)C3CCC(F)(F)CC3)nc2c1]

The title compounds were prepared as described for the synthesis of Example 78, using 2-(fluoromethyl)cyclobutane-1-carboxylic acid in place of 2-((3-cyanobicyclo[1.1.1]pentan-1-yl)methyl)-2H-1,2,3-triazole-4-carboxylic acid to provide the title compounds as a mixture of cis and trans diastereomers. The diastereomers were separated by silica gel chromatography (0-100% EtOAc/hexanes) followed by preparative HPLC (Boston Prime, C18, 250×50 mm, 5 μM, 10-100% MeCN/water with 20 mM NH₃) and then chiral preparative SFC (ChiralPak IA, 2×25 mm, isocratic elution with 40% (4:1 heptane/ethanol with 0.1% Et₂NH)/CO₂ at 100 bar) to afford the title compounds as two mixed fractions: the cis-diastereomers, and the trans-diastereomers. Example 113 was the first eluting fraction (a mixture of cis-diastereomers), isolated as a white solid. Example 114 was the second eluting fraction (a mixture of trans-diastereomers), isolated as a white solid. Example 113: ¹H NMR (500 MHz, CD₃OD) δ 7.49 (s, 2H), 7.22 (dd, J=8.4, 1.6 Hz, 1H), 5.10 (q, J=7.0 Hz, 1H), 5.04 (d, J=8.4 Hz, 1H), 4.42-4.32 (m, 1H), 4.32-4.22 (m, 1H), 3.14-3.04 (m, 1H), 2.91-2.74 (m, 1H), 2.56-2.35 (m, 4H), 2.24-1.64 (m, 10H), 1.57-1.46 (m, 4H), 1.46-1.26 (m, 2H). MS (ESI) m/z: [M+H]⁺ Found 547.2. Example 114: ¹H NMR (500 MHz, CD₃OD) δ 7.49 (s, 2H), 7.22 (dd, J=8.4, 1.6 Hz, 1H), 5.10 (q, J=7.0 Hz, 1H), 5.05 (d, J=8.4 Hz, 1H), 4.50-4.39 (m, 1H), 4.39-4.28 (m, 1H), 3.14-3.03 (m, 1H), 2.91-2.74 (m, 1H), 2.56-2.35 (m, 4H), 2.23-1.63 (m, 10H), 1.56-1.46 (m, 4H), 1.46-1.23 (m, 2H). MS (ESI) m/z: [M+H]⁺ Found 547.2.

### Example 115

### N—((R)-1-(2-((S)-(4,4-Difluorocyclohexyl)(isobutyramido)methyl)-1H-benzo[d]imidazol-5-yl)ethyl)-4,4,4-trifluorobutanamide

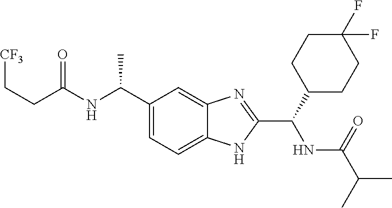 [SMILES: CC(C)C(=O)N[C@H](c1nc2cc([C@@H](C)NC(=O)CCC(F)(F)F)ccc2[nH]1)C1CCC(F)(F)CC1]

N—((R)-1-(2-((S)-Amino(4,4-difluorocyclohexyl)methyl)-1H-benzo[d]imidazol-5-yl)ethyl)-4,4,4-trifluorobutanamide (26.6 mg, 0.0523 mmol, Intermediate 4), ACN (1.2 mL), and DIPEA (40 L, 0.23 mmol) were added to a 4 mL vial, and the resultant mixture stirred until homogeneous prior to adding isobutyric anhydride (12.7 mg, 0.0803 mmol). The resultant mixture was stirred for 20.5 h before it was concentrated to dryness and the crude product subjected to silica gel chromatography (0-100% EtOAc/hexanes) followed by preparative HPLC (Boston Prime, C18, 250×50 mm, 5 μM, 10-100% MeCN/water with 20 mM NH₃) to provide the title compound as a white solid. ¹H NMR (500 MHz, CD₃OD) δ 7.66-7.30 (m, 2H), 7.23 (dd, J=8.4, 1.7 Hz, 1H), 5.11 (q, J=7.0 Hz, 1H), 5.03 (d, J=8.5 Hz, 1H), 2.64-2.53 (m, 1H), 2.53-2.37 (m, 4H), 2.22-2.04 (m, 2H), 2.04-1.89 (m, 2H), 1.88-1.65 (m, 2H), 1.56-1.40 (m, 5H), 1.39-1.26 (m, 1H), 1.15 (d, J=6.9 Hz, 3H), 1.08 (d, J=6.8 Hz, 3H). MS (ESI) m/z: [M+H]⁺ Found 503.1.

### Example 116

### N—((S)-(4,4-Difluorocyclohexyl)(5-((R)-1-(4,4,4-trifluoro-3-(trifluoromethyl)butanamido)ethyl)-1H-benzo[d]imidazol-2-yl)methyl)-1-methyl-1H-pyrazole-5-carboxamide

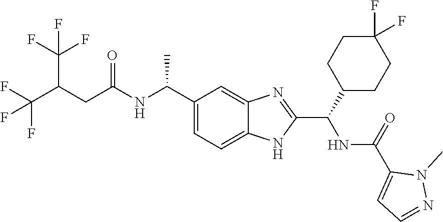 [SMILES: C[C@@H](NC(=O)CC(C(F)(F)F)C(F)(F)F)c1ccc2[nH]c([C@@H](NC(=O)c3ccnn3C)C3CCC(F)(F)CC3)nc2c1]

A vial was charged with 4,4,4-trifluoro-3-(trifluoromethyl)butanoic acid (30 mg, 0.14 mmol), N—((S)-(6-((R)-1-aminoethyl)-1H-benzo[d]imidazol-2-yl)(4,4-difluorocyclohexyl)methyl)-1-methyl-1H-pyrazole-5-carboxamide (50 mg, 0.11 mmol, Intermediate 155), HATU (55 mg, 0.14 mmol), DIPEA (38 μL, 0.22 mmol) and DMF (1 mL). The reaction was stirred at rt for 1 h, then the mixture was poured over water and extracted with EtOAc (3×5 mL). The combined organic extracts were washed with water, 10% aqueous LiCl and brine, dried over anhydrous MgSO₄, filtered, and concentrated to dryness. The crude material was purified by silica gel chromatography (0-100% EtOAc (with 10% MeOH)/hexanes). The product containing fractions were condensed into a glassy solid which was further purified by acidic preparative HPLC (SunFire® Prep C18, OBDTM, 5 μm, 30×250 mm column; 0-100% acetonitrile (0.05% TFA)/water (0.05% TFA)). The fractions containing product were frozen and lyophilized to afford the title compound as a white powder. ¹H NMR (400 MHz, DMSO-d₆, benzimidazole NH absent from exchange) δ 9.11 (d, J=6.9 Hz, 1H), 8.79 (d, J=7.6 Hz, 1H), 7.70 (d, J=8.5 Hz, 1H), 7.62 (s, 1H), 7.52 (d, J=2.0 Hz, 1H), 7.42 (d, J=7.9 Hz, 1H), 7.09 (d, J=2.0 Hz, 1H), 5.27 (t, J=7.4 Hz, 1H), 5.15-5.03 (m, 1H), 4.27-4.09 (m, 1H), 3.99 (s, 3H), 2.84-2.66 (m, 2H), 2.40-2.23 (m, 1H), 2.18-1.97 (m, 3H), 1.95-1.68 (m, 2H), 1.66-1.55 (m, 1H), 1.49-1.19 (m, 5H). MS (ESI) m/z: [M+H]⁺ Found 609.3.

### Example 117

### N—((S)-(4,4-Difluorocyclohexyl)(5-((R)-1-(3,3-dimethylbutanamido)ethyl)-1H-benzo[d]imidazol-2-yl)methyl)-1-methyl-1H-pyrazole-5-carboxamide

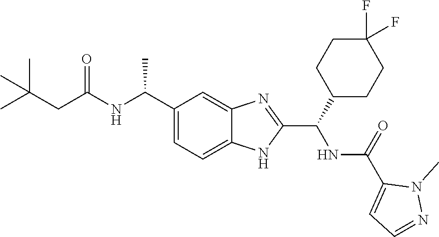 [SMILES: C[C@@H](NC(=O)CC(C)(C)C)c1ccc2[nH]c([C@@H](NC(=O)c3ccnn3C)C3CCC(F)(F)CC3)nc2c1]

The title compound was prepared as described for Example 116, using 3,3-dimethylbutyric acid in place of 4,4,4-trifluoro-3-(trifluoromethyl)butanoic acid to afford the title compound as a white powder. ¹H NMR (400 MHz, DMSO-d₆, benzimidazole NH absent from exchange) δ 9.07 (d, J=7.1 Hz, 1H), 8.28 (d, J=7.9 Hz, 1H), 7.67 (d, J=8.5 Hz, 1H), 7.60 (s, 1H), 7.52 (d, J=2.0 Hz, 1H), 7.44-7.37 (m, 1H), 7.09 (d, J=2.1 Hz, 1H), 5.26 (t, J=7.4 Hz, 1H), 5.12-4.99 (m, 1H), 4.00 (s, 3H), 2.37-2.26 (m, 1H), 2.16-1.98 (m, 5H), 1.93-1.70 (m, 2H), 1.65-1.56 (m, 1H), 1.47-1.22 (m, 5H), 0.94 (s, 9H). MS (ESI) m/z: [M+H]⁺ Found 515.3.

### Example 118

### N—((S)-(4,4-Difluorocyclohexyl)(5-((R)-1-(4,4,4-trifluorobutanamido)ethyl)-1H-benzo[d]imidazol-2-yl)methyl)-1-methyl-1H-pyrazole-5-carboxamide

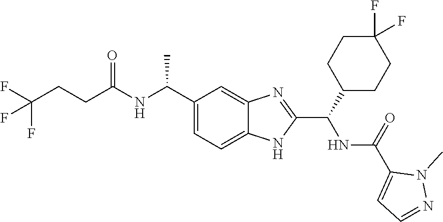 [SMILES: C[C@@H](NC(=O)CCC(F)(F)F)c1ccc2[nH]c([C@@H](NC(=O)c3ccnn3C)C3CCC(F)(F)CC3)nc2c1]

The title compound was prepared as described for Example 116, using 4,4,4-trifluorobutyric acid in place of 4,4,4-trifluoro-3-(trifluoromethyl)butanoic acid to afford the title compound as a white powder. ¹H NMR (400 MHz, DMSO-d₆, benzimidazole NH absent from exchange) δ 9.12-8.98 (m, 1H), 8.56 (d, J=7.6 Hz, 1H), 7.66 (d, J=8.4 Hz, 1H), 7.59 (s, 1H), 7.52 (d, J=2.0 Hz, 1H), 7.44-7.31 (m, 1H), 7.09 (d, J=2.1 Hz, 1H), 5.24 (t, J=7.5 Hz, 1H), 5.13-4.98 (m, 1H), 4.00 (s, 3H), 2.46-2.27 (m, 5H), 2.15-1.97 (m, 3H), 1.93-1.70 (m, 2H), 1.64-1.55 (m, 1H), 1.48-1.23 (m, 5H). MS (ESI) m/z: [M+H]⁺ Found 541.2.

### Example 119

### N—((S)-(5-((R)-1-(2-(3,3-Difluorocyclobutyl)acetamido)ethyl)-1H-benzo[d]imidazol-2-yl)(4,4-difluorocyclohexyl)methyl)-1-methyl-1H-pyrazole-5-carboxamide

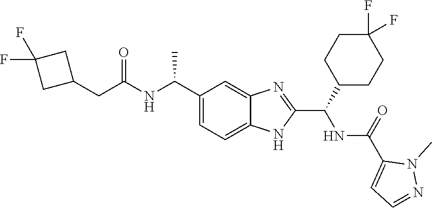 [SMILES: C[C@@H](NC(=O)CC1CC(F)(F)C1)c1ccc2[nH]c([C@@H](NC(=O)c3ccnn3C)C3CCC(F)(F)CC3)nc2c1]

The title compound was prepared as described for Example 116, using 2-(3,3-difluorocyclobutyl)acetic acid in place of 4,4,4-trifluoro-3-(trifluoromethyl)butanoic acid to afford the title compound as a white powder. ¹H NMR (400 MHz, DMSO-d₆, benzimidazole NH absent from exchange) δ 9.14-9.03 (m, 1H), 8.45 (d, J=7.6 Hz, 1H), 7.68 (d, J=8.5 Hz, 1H), 7.58 (s, 1H), 7.52 (d, J=2.1 Hz, 1H), 7.44-7.31 (m, 1H), 7.09 (d, J=2.1 Hz, 1H), 5.25 (t, J=7.5 Hz, 1H), 5.10-4.95 (m, 1H), 3.99 (s, 3H), 2.72-2.57 (m, 2H), 2.43-2.22 (m, 6H), 2.16-1.97 (m, 3H), 1.94-1.69 (m, 2H), 1.64-1.55 (m, 1H), 1.48-1.21 (m, 5H). MS (ESI) m/z: [M+H]⁺ Found 549.3.

### Example 120

### N—((S)-(4,4-Difluorocyclohexyl)(6-((R)-1-(4,4,4-trifluorobutanamido)ethyl)-1H-benzo[d]imidazol-2-yl)methyl)benzamide

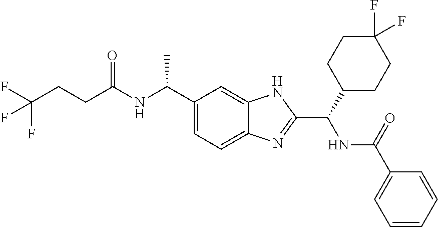 [SMILES: C[C@@H](NC(=O)CCC(F)(F)F)c1ccc2nc([C@@H](NC(=O)c3ccccc3)C3CCC(F)(F)CC3)[nH]c2c1]

A flask was charged with N—((S)-(4,4-difluorocyclohexyl)(6-((R)-1-(4,4,4-trifluorobutanamido)ethyl)-1-((2-(trimethylsilyl)ethoxy)methyl)-1H-benzo[d]imidazol-2-yl)methyl)benzamide and N—((S)-(4,4-difluorocyclohexyl)(5-((R)-1-(4,4,4-trifluorobutanamido)ethyl)-1-((2-(trimethylsilyl)ethoxy)methyl)-1H-benzo[d]imidazol-2-yl)methyl)benzamide (80 mg, 0.12 mmol, Intermediate 150) and TFA (2 mL). The reaction was stirred at rt for 30 min. Then the reaction mixture was condensed and dissolved in a minimal amount of methanol (approximately 1.5 mL) and purified directly by acidic preparative HPLC (SunFire® Prep, C18, OBDTM, 5 μm, 30×250 mm column; 0-100% acetonitrile (0.05% TFA)/water (0.05% TFA)). The product containing fractions were frozen and lyophilized to afford the title compound as a white powder. ¹H NMR (400 MHz, DMSO-d₆ benzimidazole NH absent from exchange) δ 9.14-9.03 (m, 1H), 8.57 (d, J=7.8 Hz, 1H), 7.99-7.89 (m, 2H), 7.67 (d, J=8.3 Hz, 1H), 7.64-7.56 (m, 2H), 7.55-7.48 (m, 2H), 7.44-7.36 (m, 1H), 5.27 (t, J=7.4 Hz, 1H), 5.13-4.97 (m, 1H), 2.48-2.27 (m, 5H), 2.17-1.98 (m, 3H), 1.94-1.70 (m, 2H), 1.66-1.56 (m, 1H), 1.48-1.37 (m, 4H), 1.36-1.22 (m, 1H). MS (ESI) m/z: [M+H]⁺ Found 537.3.

### Example 121

### N—((S)-(4,4-Difluorocyclohexyl)(6-((R)-1-(4,4,4-trifluorobutanamido)ethyl)-1H-benzo[d]imidazol-2-yl)methyl)-1-(3,3,3-trifluoropropyl)-1H-pyrazole-5-carboxamide

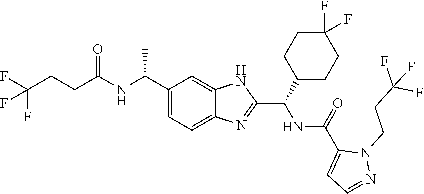 [SMILES: C[C@@H](NC(=O)CCC(F)(F)F)c1ccc2nc([C@@H](NC(=O)c3ccnn3CCC(F)(F)F)C3CCC(F)(F)CC3)[nH]c2c1]

A vial was charged with N—((R)-1-(2-((S)-amino(4,4-difluorocyclohexyl)methyl)-1H-benzo[d]imidazol-6-yl)ethyl)-4,4,4-trifluorobutanamide (50 mg, 0.12 mmol, Intermediate 4), 1-(3,3,3-trifluoropropyl)-1H-pyrazole-5-carboxylic acid (39 mg, 0.15 mmol), bis(2-oxo-3-oxazolidinyl)phosphinic chloride (31 mg, 0.15 mmol), DMF (1 mL) and DIPEA (40 μL, 0.23 mmol). The reaction was stirred at rt for 30 min. The reaction mixture was poured over water and extracted with EtOAc (3×5 mL). The combined extracts were washed with water, 10% aqueous LiCl, then brine, dried over anhydrous MgSO₄, filtered and condensed. The crude material was purified by silica gel chromatography (0-100% (EtOAc with 10% MeOH)/hexanes). The product containing fractions were condensed into a glassy solid and further purified by acidic preparative HPLC (SunFire® Prep, C18, OBDTM, 5 μm, 30×250 mm column; 0-100% acetonitrile (0.05% TFA)/water (0.05% TFA)). The product containing fractions were frozen and lyophilized to afford the title compound as a white powder. ¹H NMR (400 MHz, DMSO-d₆ benzimidazole NH absent from exchange) δ 9.16 (d, J=7.1 Hz, 1H), 8.57 (d, J=7.8 Hz, 1H), 7.67 (d, J=8.5 Hz, 1H), 7.60 (s, 2H), 7.39 (d, J=7.9 Hz, 1H), 7.14 (d, J=2.1 Hz, 1H), 5.27 (t, J=7.6 Hz, 1H), 5.11-5.01 (m, 1H), 4.83-4.60 (m, 2H), 2.86-2.71 (m, 2H), 2.49-2.26 (m, 5H), 2.15-1.97 (m, 3H), 1.93-1.69 (m, 2H), 1.64-1.54 (m, 1H), 1.40 (d, J=7.0 Hz, 4H), 1.35-1.22 (m, 1H). MS (ESI) m/z: [M+H]⁺ Found 623.3.

### Example 122

### N—((S)-(4,4-Difluorocyclohexyl)(6-((R)-1-(4,4,4-trifluorobutanamido)ethyl)-1H-benzo[d]imidazol-2-yl)methyl)-1-isopropyl-1H-pyrazole-5-carboxamide

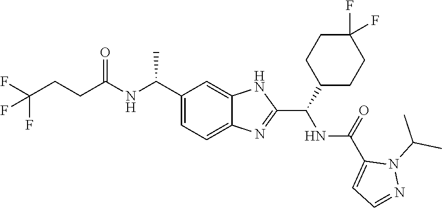 [SMILES: CC(C)n1nccc1C(=O)N[C@H](c1nc2ccc([C@@H](C)NC(=O)CCC(F)(F)F)cc2[nH]1)C1CCC(F)(F)CC1]

The title compound was prepared as described for Example 121, using 1-isopropyl-1H-pyrazole-5-carboxylic acid in place of 1-(3,3,3-trifluoropropyl)-1H-pyrazole-5-carboxylic acid to afford the title compound as a white powder. ¹H NMR (400 MHz, DMSO-d₆ benzimidazole NH absent from exchange) δ 9.04 (d, J=7.1 Hz, 1H), 8.56 (d, J=7.8 Hz, 1H), 7.65 (d, J=8.5 Hz, 1H), 7.58 (s, 1H), 7.55 (d, J=2.0 Hz, 1H), 7.38 (s, 1H), 7.03 (d, J=2.0 Hz, 1H), 5.39-5.27 (m, 1H), 5.23 (t, J=7.6 Hz, 1H), 5.11-5.00 (m, 1H), 2.49-2.24 (m, 5H), 2.15-1.97 (m, 3H), 1.93-1.69 (m, 2H), 1.64-1.55 (m, 1H), 1.40 1.47-1.37 (m, 4H), 1.37-1.22 (m, 7H). MS (ESI) m/z: [M+H]⁺ Found 569.3.

### Example 123

### N—((S)-(4,4-Difluorocyclohexyl)(6-((R)-1-(4,4,4-trifluorobutanamido)ethyl)-1H-benzo[d]imidazol-2-yl)methyl)-2,5-difluorobenzamide

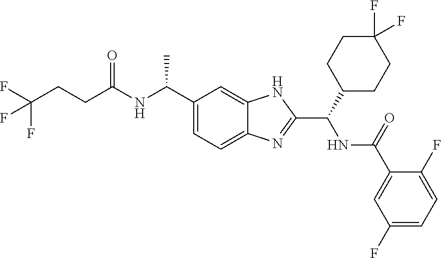 [SMILES: C[C@@H](NC(=O)CCC(F)(F)F)c1ccc2nc([C@@H](NC(=O)c3cc(F)ccc3F)C3CCC(F)(F)CC3)[nH]c2c1]

The title compound was prepared as described for Example 121, using 2,5-difluorobenzoic acid in place of 1-(3,3,3-trifluoropropyl)-1H-pyrazole-5-carboxylic acid to afford the title compound as a white powder. ¹H NMR (400 MHz, DMSO-d₆ benzimidazole NH absent from exchange) δ 9.30-9.09 (m, 1H), 8.57 (d, J=7.8 Hz, 1H), 7.67 (d, J=8.5 Hz, 1H), 7.61 (s, 1H), 7.57-7.51 (m, 1H), 7.48-7.36 (m, 3H), 5.28 (t, J=7.3 Hz, 1H), 5.12-5.00 (m, 1H), 2.49-2.21 (m, 5H), 2.14-1.96 (m, 3H), 1.92-1.70 (m, 2H), 1.64-1.54 (m, 1H), 1.50-1.27 (m, 5H). MS (ESI) m/z: [M+H]⁺ Found 573.2.

### Example 124

### N—((S)-(4,4-Difluorocyclohexyl)(5-((S)-1-(4,4,4-trifluorobutanamido)ethyl)-1H-benzo[d]imidazol-2-yl)methyl)-1-(3,3,3-trifluoropropyl)-1H-pyrazole-5-carboxamide

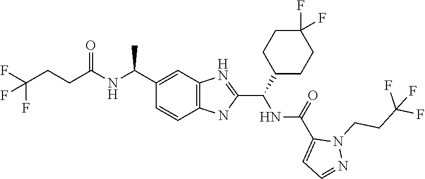

The title compound was prepared as described for Example 121, using N—((S)-1-(2-((S)-amino(4,4-difluorocyclohexyl)methyl)-1H-benzo[d]imidazol-6-yl)ethyl)-4,4,4-trifluorobutanamide (Intermediate 152) in place of N—((R)-1-(2-((S)-amino(4,4-difluorocyclohexyl)methyl)-1H-benzo[d]imidazol-6-yl)ethyl)-4,4,4-trifluorobutanamide to afford the title compound as a white powder. ¹H NMR (400 MHz, DMSO-d₆ benzimidazole NH absent from exchange) δ 9.23 (d, J=7.0 Hz, 1H), 8.60 (d, J=7.6 Hz, 1H), 7.71 (d, J=8.5 Hz, 1H), 7.64 (s, 1H), 7.61 (d, J=2.0 Hz, 1H), 7.47-7.41 (m, 1H), 7.14 (d, J=2.0 Hz, 1H), 5.30 (t, J=7.4 Hz, 1H), 5.11-5.02 (m, 1H), 4.81-4.68 (m, 1H), 4.80-4.68 (m, 2H), 2.91-2.70 (m, 2H), 2.49-2.28 (m, 5H), 2.16-1.98 (m, 3H), 1.93-1.69 (m, 2H), 1.64-1.54 (m, 1H), 1.47-1.23 (m, 4H). MS (ESI) m/z: [M+H]⁺ Found 623.3.

### Example 125

### N—((S)-(4,4-Difluorocyclohexyl)(5-((R)-1-(4,4,4-trifluoro-3-(trifluoromethyl)butanamido)ethyl)-1H-benzo[d]imidazol-2-yl)methyl)-1-isopropyl-1H-pyrazole-5-carboxamide

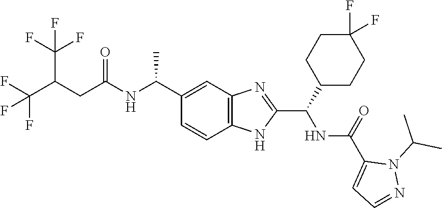 [SMILES: CC(C)n1nccc1C(=O)N[C@H](c1nc2cc([C@@H](C)NC(=O)CC(C(F)(F)F)C(F)(F)F)ccc2[nH]1)C1CCC(F)(F)CC1]

A vial was charged with a stir bar, 1-isopropyl-1H-pyrazole-5-carboxylic acid (30 mg, 0.2 mmol), HATU (76 mg, 0.2 mmol) and DMF (1 mL). The reaction was stirred at rt for 5 min then N—((R)-1-(2-((S)-amino(4,4-difluorocyclohexyl)methyl)-1H-benzo[d]imidazol-5-yl)ethyl)-4,4,4-trifluoro-3-(trifluoromethyl)butanamide (75 mg, 0.15 mmol, Intermediate 154) and DIPEA (52 μL, 0.3 mmol) were added and the reaction was stirred for 30 min at rt. The reaction was poured over water and extracted with EtOAc (3×5 mL). The combined organics were washed with 10% aqueous LiCl, water and brine, dried over anhydrous MgSO₄, filtered and condensed. The crude material was purified by silica gel chromatography (0-100% (EtOAc with 10% MeOH)/hexanes). The product containing fractions were condensed into a glassy solid and further purified by acidic preparative HPLC (SunFire® Prep, C18, OBDTM, 5 μm, 30×250 mm column; 0-100% acetonitrile (0.05% TFA)/water (0.05% TFA). The product containing fractions were frozen and lyophilized to yield the title compound as a white powder. ¹H NMR (400 MHz, DMSO-d₆ benzimidazole NH absent from exchange) δ 9.06-9.00 (m, 1H), 8.77 (d, J=7.6 Hz, 1H), 7.66 (d, J=8.4 Hz, 1H), 7.59 (s, 1H), 7.55 (d, J=1.9 Hz, 1H), 7.40-7.33 (m, 1H), 7.03 (d, J=1.9 Hz, 1H), 5.36-5.28 (m, 1H), 5.23 (t, J=7.6 Hz, 1H), 5.13-5.04 (m, 1H), 4.26-4.13 (m, 1H), 2.83-2.66 (m, 2H), 2.36-2.24 (m, 1H), 2.16-1.96 (m, 3H), 1.93-1.69 (m, 2H), 1.65-1.55 (m, 1H), 1.47-1.38 (m, 4H), 1.37-1.31 (m, 6H), 1.30-1.22 (m, 1H). MS (ESI) m/z: [M+H]⁺ Found 637.3.

### Example 126

### N—((S)-(4,4-Difluorocyclohexyl)(5-((R)-1-(4,4,4-trifluoro-3-(trifluoromethyl)butanamido)ethyl)-1H-benzo[d]imidazol-2-yl)methyl)-1-(3,3,3-trifluoropropyl)-1H-pyrazole-5-carboxamide

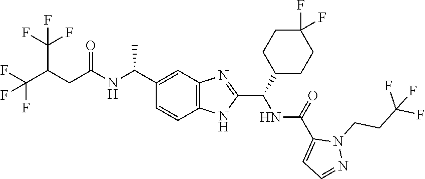 [SMILES: C[C@@H](NC(=O)CC(C(F)(F)F)C(F)(F)F)c1ccc2[nH]c([C@@H](NC(=O)c3ccnn3CCC(F)(F)F)C3CCC(F)(F)CC3)nc2c1]

The title compound was prepared as described in Example 125, using 1-(3,3,3-trifluoropropyl)-1H-pyrazole-5-carboxylic acid in place of 1-isopropyl-1H-pyrazole-5-carboxylic acid to afford the title compound as a white powder. ¹H NMR (400 MHz, DMSO-d₆ benzimidazole NH absent from exchange) δ 9.14 (d, J=7.3 Hz, 1H), 8.76 (d, J=7.6 Hz, 1H), 7.66 (d, J=8.4 Hz, 1H), 7.62-7.56 (m, 2H), 7.40-7.33 (m, 1H), 7.14 (d, J=2.0 Hz, 1H), 5.26 (t, J=7.7 Hz, 1H), 5.13-5.03 (m, 1H), 4.81-4.57 (m, 2H), 4.28-4.07 (m, 1H), 2.86-2.66 (m, 4H), 2.38-2.26 (m, 1H), 2.16-1.97 (m, 3H), 1.93-1.69 (m, 2H), 1.64-1.54 (m, 1H), 1.47-1.35 (m, 4H), 1.37-1.23 (m, 1H). MS (ESI) m/z: [M+H]⁺ Found 691.3.

### Example 127

### N—((S)-(4,4-Difluorocyclohexyl)(5-((R)-1-(4,4,4-trifluoro-3-(trifluoromethyl)butanamido)ethyl)-1H-benzo[d]imidazol-2-yl)methyl)-1-ethyl-1H-pyrazole-5-carboxamide

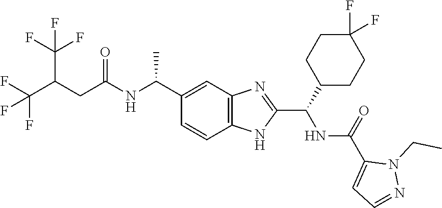 [SMILES: CCn1nccc1C(=O)N[C@H](c1nc2cc([C@@H](C)NC(=O)CC(C(F)(F)F)C(F)(F)F)ccc2[nH]1)C1CCC(F)(F)CC1]

The title compound was prepared as described in Example 125, using 1-ethyl-1H-pyrazole-5-carboxylic acid in place of 1-isopropyl-1H-pyrazole-5-carboxylic acid to afford the title compound as a white powder. ¹H NMR (400 MHz, DMSO-d₆ benzimidazole NH absent from exchange) δ 9.04 (d, J=7.3 Hz, 1H), 8.76 (d, J=7.6 Hz, 1H), 7.65 (d, J=8.4 Hz, 1H), 7.58 (s, 1H), 7.53 (d, J=2.0 Hz, 1H), 7.38-7.32 (m, 1H), 7.07 (d, J=2.1 Hz, 1H), 5.24 (t, J=7.6 Hz, 1H), 5.14-5.02 (m, 1H), 4.50-4.37 (m, 2H), 4.26-4.09 (m, 1H), 2.85-2.67 (m, 2H), 2.37-2.25 (m, 1H), 2.16-1.96 (m, 3H), 1.93-1.69 (m, 2H), 1.65-1.55 (m, 1H), 1.48-1.35 (m, 4H), 1.33-1.22 (m, 4H). MS (ESI) m/z: [M+H]⁺ Found 623.3.

### Example 128

### N—((S)-(4,4-Difluorocyclohexyl)(5-((R)-1-(4,4,4-trifluorobutanamido)ethyl)-1H-benzo[d]imidazol-2-yl)methyl)-1-(2,2,2-trifluoroethyl)-1H-pyrazole-5-carboxamide

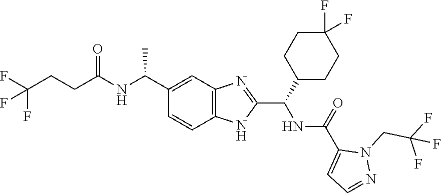 [SMILES: C[C@@H](NC(=O)CCC(F)(F)F)c1ccc2[nH]c([C@@H](NC(=O)c3ccnn3CC(F)(F)F)C3CCC(F)(F)CC3)nc2c1]

The title compound was prepared as described for Example 121, using 1-(2,2,2-trifluoroethyl)-1H-pyrazole-5-carboxylic acid in place of 1-(3,3,3-trifluoropropyl)-1H-pyrazole-5-carboxylic acid to afford the title compound as a white powder. ¹H NMR (400 MHz, DMSO-d₆ benzimidazole NH absent from exchange) δ 9.25 (d, J=7.3 Hz, 1H), 8.54 (d, J=7.9 Hz, 1H), 7.73 (d, J=2.0 Hz, 1H), 7.64 (d, J=8.4 Hz, 1H), 7.57 (s, 1H), 7.38-7.31 (m, 1H), 7.26 (d, J=2.1 Hz, 1H), 5.56-5.34 (m, 2H), 5.24 (t, J=7.8 Hz, 1H), 5.11-5.00 (m, 1H), 2.48-2.26 (m, 5H), 2.15-1.95 (m, 3H), 1.93-1.70 (m, 2H), 1.65-1.53 (m, 1H), 1.47-1.22 (m, 5H). MS (ESI) m/z: [M+H]⁺ Found 609.2.

### Example 129

### N—((S)-(4,4-Difluorocyclohexyl)(5-((R)-1-(4,4,4-trifluoro-3-(trifluoromethyl)butanamido)ethyl)-1H-benzo[d]imidazol-2-yl)methyl)-1-(2,2,2-trifluoroethyl)-1H-pyrazole-5-carboxamide

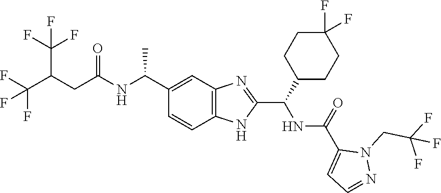 [SMILES: C[C@@H](NC(=O)CC(C(F)(F)F)C(F)(F)F)c1ccc2[nH]c([C@@H](NC(=O)c3ccnn3CC(F)(F)F)C3CCC(F)(F)CC3)nc2c1]

The title compound was prepared as described for Example 121, using 1-(2,2,2-trifluoroethyl)-1H-pyrazole-5-carboxylic acid in place of 1-(3,3,3-trifluoropropyl)-1H-pyrazole-5-carboxylic acid and N—((R)-1-(2-((S)-amino(4,4-difluorocyclohexyl)methyl)-1H-benzo[d]imidazol-6-yl)ethyl)-4,4,4-trifluoro-3-(trifluoromethyl)butanamide (Intermediate 154) in place of N—((R)-1-(2-((S)-amino(4,4-difluorocyclohexyl)methyl)-1H-benzo[d]imidazol-6-yl)ethyl)-4,4,4-trifluorobutanamide to afford the title compound as a white powder. ¹H NMR (400 MHz, DMSO-d₆ benzimidazole NH absent from exchange) δ 9.27 (d, J=7.3 Hz, 1H), 8.76 (d, J=7.6 Hz, 1H), 7.73 (d, J=2.0 Hz, 1H), 7.66 (d, J=8.4 Hz, 1H), 7.58 (s, 1H), 7.39-7.33 (m, 1H), 7.25 (d, J=2.1 Hz, 1H), 5.54-5.33 (m, 2H), 5.25 (t, J=7.8 Hz, 1H), 5.14-5.02 (m, 1H), 4.24-4.12 (m, 1H), 2.84-2.69 (m, 2H), 2.36-2.26 (m, 1H), 2.16-1.96 (m, 3H), 1.93-1.69 (m, 2H), 1.64-1.54 (m, 1H), 1.47-1.22 (m, 5H). MS (ESI) m/z: [M+H]⁺ Found 677.2.

### Example 130

### N—((S)-(4,4-Difluorocyclohexyl)(6-((R)-1-(4,4,4-trifluoro-3-(trifluoromethyl)butanamido)ethyl)-1H-benzo[d]imidazol-2-yl)methyl)-2,5-difluorobenzamide

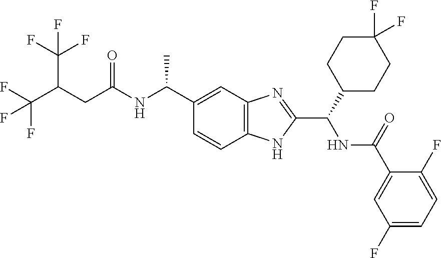 [SMILES: C[C@@H](NC(=O)CC(C(F)(F)F)C(F)(F)F)c1ccc2[nH]c([C@@H](NC(=O)c3cc(F)ccc3F)C3CCC(F)(F)CC3)nc2c1]

The title compound was prepared as described in Example 121, using N—((R)-1-(2-((S)-amino(4,4-difluorocyclohexyl)methyl)-1H-benzo[d]imidazol-6-yl)ethyl)-4,4,4-trifluoro-3-(trifluoromethyl)butanamide (Intermediate 154) in place of N—((R)-1-(2-((S)-amino(4,4-difluorocyclohexyl)methyl)-1H-benzo[d]imidazol-6-yl)ethyl)-4,4,4-trifluorobutanamide and 2,5-difluorobenzoic acid in place of 1-(3,3,3-trifluoropropyl)-1H-pyrazole-5-carboxylic acid to afford the title compound as a white powder. ¹H NMR (400 MHz, DMSO-d₆ benzimidazole NH absent from exchange) δ 9.18-9.07 (m, 1H), 8.74 (d, J=7.8 Hz, 1H), 7.67-7.60 (m, 1H), 7.58 (s, 1H), 7.56-7.49 (m, 1H), 7.48-7.36 (m, 2H), 7.35-7.29 (m, 1H), 5.25 (t, J=7.6 Hz, 1H), 5.13-5.03 (m, 1H), 4.26-4.14 (m, 1H), 2.83-2.69 (m, 2H), 2.31-2.21 (m, 1H), 2.14-1.96 (m, 3H), 1.91-1.70 (m, 2H), 1.63-1.53 (m, 1H), 1.49-1.28 (m, 5H). MS (ESI) m/z: [M+H]⁺ Found 641.2.

### Example 131

### 3-Cyano-N—((S)-(4,4-difluorocyclohexyl)(6-((R)-1-(4,4,4-trifluorobutanamido)ethyl)-1H-benzo[d]imidazol-2-yl)methyl)benzamide

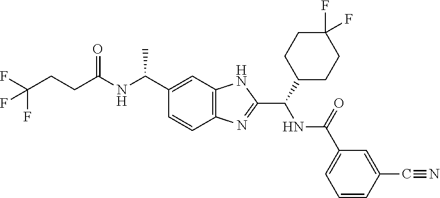 [SMILES: C[C@@H](NC(=O)CCC(F)(F)F)c1ccc2nc([C@@H](NC(=O)c3cccc(C#N)c3)C3CCC(F)(F)CC3)[nH]c2c1]

The title compound was prepared as described in Example 121, using 3-cyanobenzoic acid in place of 1-(3,3,3-trifluoropropyl)-1H-pyrazole-5-carboxylic acid to afford the title compound as a white powder. ¹H NMR (400 MHz, DMSO-d₆ benzimidazole NH absent from exchange) δ 9.30-9.23 (m, 1H), 8.54 (d, J=7.8 Hz, 1H), 8.44-8.40 (m, 1H), 8.23-8.16 (m, 1H), 8.09-8.04 (m, 1H), 7.73 (t, J=7.9 Hz, 1H), 7.64 (d, J=8.3 Hz, 1H), 7.58 (s, 1H), 7.39-7.32 (m, 1H), 5.26 (t, J=7.3 Hz, 1H), 5.10-5.01 (m, 1H), 2.48-2.36 (m, 4H), 2.35-2.27 (m, 1H), 2.14-1.98 (m, 3H), 1.92-1.71 (m, 2H), 1.67-1.57 (m, 1H), 1.50-1.24 (m, 5H). MS (ESI) m/z: [M+H]⁺ Found 562.3.

### Example 132

### 3-Cyano-N—((S)-(4,4-difluorocyclohexyl)(6-((R)-1-(4,4,4-trifluoro-3-(trifluoromethyl)butanamido)ethyl)-1H-benzo[d]imidazol-2-yl)methyl)benzamide

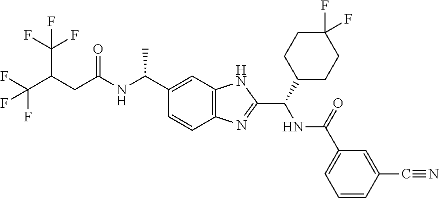 [SMILES: C[C@@H](NC(=O)CC(C(F)(F)F)C(F)(F)F)c1ccc2nc([C@@H](NC(=O)c3cccc(C#N)c3)C3CCC(F)(F)CC3)[nH]c2c1]

The title compound was prepared as described in Example 121, using N—((R)-1-(2-((S)-amino(4,4-difluorocyclohexyl)methyl)-1H-benzo[d]imidazol-6-yl)ethyl)-4,4,4-trifluoro-3-(trifluoromethyl)butanamide (Intermediate 154) in place of N—((R)-1-(2-((S)-amino(4,4-difluorocyclohexyl)methyl)-1H-benzo[d]imidazol-6-yl)ethyl)-4,4,4-trifluorobutanamide and 3-cyanobenzoic acid in place of 1-(3,3,3-trifluoropropyl)-1H-pyrazole-5-carboxylic acid to afford the title compound as a white powder. ¹H NMR (400 MHz, DMSO-d₆ benzimidazole NH absent from exchange) δ 9.27 (d, J=7.0 Hz, 1H), 8.75 (d, J=7.6 Hz, 1H), 8.42 (t, J=1.4 Hz, 1H), 8.25-8.14 (m, 1H), 8.12-8.00 (m, 1H), 7.73 (t, J=7.9 Hz, 1H), 7.65 (d, J=8.4 Hz, 1H), 7.58 (s, 1H), 7.39-7.31 (m, 1H), 5.26 (t, J=7.4 Hz, 1H), 5.13-5.02 (m, 1H), 4.27-4.12 (m, 1H), 2.84-2.70 (m, 2H), 2.37-2.25 (m, 1H), 2.16-1.99 (m, 3H), 1.95-1.72 (m, 2H), 1.67-1.56 (m, 1H), 1.49-1.23 (m, 5H). MS (ESI) m/z: [M+H]⁺ Found 630.3.

### Example 133

### N—((S)-(4,4-Difluorocyclohexyl)(6-((R)-1-((R*)-4,4,4-trifluoro-3-methylbutanamido)ethyl)-1H-benzo[d]imidazol-2-yl)methyl)-1-(2,2,2-trifluoroethyl)-1H-pyrazole-5-carboxamide

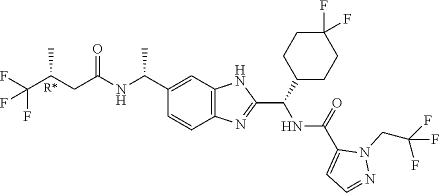 [SMILES: C[C@H](CC(=O)N[C@H](C)c1ccc2nc([C@@H](NC(=O)c3ccnn3CC(F)(F)F)C3CCC(F)(F)CC3)[nH]c2c1)C(F)(F)F]

The title compound was prepared as described in Example 121, using (R*)—N—((R)-1-(2-((S)-amino(4,4-difluorocyclohexyl)methyl)-1H-benzo[d]imidazol-5-yl)ethyl)-4,4,4-trifluoro-3-methylbutanamide (Intermediate 146) in place of N—((R)-1-(2-((S)-amino(4,4-difluorocyclohexyl)methyl)-1H-benzo[d]imidazol-5-yl)ethyl)-4,4,4-trifluorobutanamide and 1-(2,2,2-trifluoroethyl)-1H-pyrazole-5-carboxylic acid in place of 1-(3,3,3-trifluoropropyl)-1H-pyrazole-5-carboxylic acid to afford the title compound as a white powder. ¹H NMR (400 MHz, DMSO-d₆ benzimidazole NH absent from exchange) δ 9.27 (d, J=7.4 Hz, 1H), 8.58 (d, J=7.8 Hz, 1H), 7.73 (d, J=2.1 Hz, 1H), 7.65 (d, J=8.5 Hz, 1H), 7.57 (s, 1H), 7.38-7.33 (m, 1H), 7.26 (d, J=2.1 Hz, 1H), 5.56-5.33 (m, 2H), 5.25 (t, J=7.7 Hz, 1H), 5.12-4.99 (m, 1H), 2.82-2.69 (m, 1H), 2.48-2.42 (m, 1H), 2.38-2.27 (m, 1H), 2.26-2.18 (m, 1H), 2.15-1.95 (m, 3H), 1.92-1.69 (m, 2H), 1.62-1.53 (m, 1H), 1.41 (d, J=6.9 Hz, 4H), 1.36-1.22 (m, 1H), 1.07 (d, J=7.0 Hz, 3H). MS (ESI) m/z: [M+H]⁺ Found 623.3.

### Example 134

### N—((S)-(4,4-Difluorocyclohexyl)(6-((R)-1-((R*)-4,4,4-trifluoro-3-methylbutanamido)ethyl)-1H-benzo[d]imidazol-2-yl)methyl)-1-(3,3,3-trifluoropropyl)-1H-pyrazole-5-carboxamide

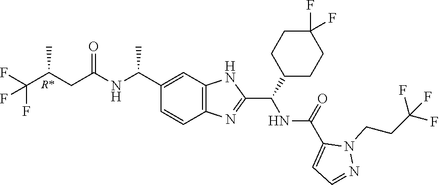 [SMILES: C[C@H](CC(=O)N[C@H](C)c1ccc2nc([C@@H](NC(=O)c3ccnn3CCC(F)(F)F)C3CCC(F)(F)CC3)[nH]c2c1)C(F)(F)F]

The title compound was prepared as described in Example 121, using (R*)—N—((R)-1-(2-((S)-amino(4,4-difluorocyclohexyl)methyl)-1H-benzo[d]imidazol-5-yl)ethyl)-4,4,4-trifluoro-3-methylbutanamide (Intermediate 146) in place of N—((R)-1-(2-((S)-amino(4,4-difluorocyclohexyl)methyl)-1H-benzo[d]imidazol-5-yl)ethyl)-4,4,4-trifluorobutanamide to afford the title compound as a white powder. ¹H NMR (400 MHz, DMSO-d₆ benzimidazole NH absent from exchange) δ 9.14-9.08 (m, 1H), 8.57 (d, J=7.8 Hz, 1H), 7.66-7.61 (m, 1H), 7.60 (d, J=2.0 Hz, 1H), 7.56 (s, 1H), 7.37-7.29 (m, 1H), 7.14 (d, J=2.1 Hz, 1H), 5.25 (t, J=7.7 Hz, 1H), 5.11-5.01 (m, 1H), 4.78-4.61 (m, 2H), 2.86-2.70 (m, 3H), 2.46-2.40 (m, 1H), 2.36-2.28 (m, 1H), 2.27-2.18 (m, 1H), 2.15-1.96 (m, 3H), 1.93-1.69 (m, 2H), 1.64-1.54 (m, 1H), 1.48-1.21 (m, 5H), 1.07 (d, J=7.0 Hz, 3H). MS (ESI) m/z: [M+H]⁺ Found 637.2.

### Example 135

### N—((S)-(4,4-Difluorocyclohexyl)(6-((R)-1-((R*)-4,4,4-trifluoro-3-methylbutanamido)ethyl)-1H-benzo[d]imidazol-2-yl)methyl)-1-isopropyl-1H-pyrazole-5-carboxamide

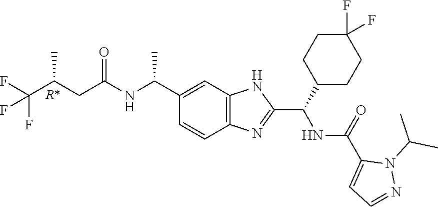 [SMILES: CC(C)n1nccc1C(=O)N[C@H](c1nc2ccc([C@@H](C)NC(=O)C[C@@H](C)C(F)(F)F)cc2[nH]1)C1CCC(F)(F)CC1]

The title compound was prepared as described in Example 121, using (R*)—N—((R)-1-(2-((S)-amino(4,4-difluorocyclohexyl)methyl)-1H-benzo[d]imidazol-5-yl)ethyl)-4,4,4-trifluoro-3-methylbutanamide (Intermediate 146) in place of N—((R)-1-(2-((S)-amino(4,4-difluorocyclohexyl)methyl)-1H-benzo[d]imidazol-5-yl)ethyl)-4,4,4-trifluorobutanamide and 1-isopropyl-1H-pyrazole-5-carboxylic acid in place of 1-(3,3,3-trifluoropropyl)-1H-pyrazole-5-carboxylic acid to afford the title compound as a white powder. ¹H NMR (400 MHz, DMSO-d₆, benzimidazole NH absent from exchange) δ 9.06 (d, J=7.0 Hz, 1H), 8.60 (d, J=7.6 Hz, 1H), 7.67 (d, J=8.5 Hz, 1H), 7.60 (s, 1H), 7.55 (d, J=2.0 Hz, 1H), 7.43-7.36 (m, 1H), 7.03 (d, J=2.0 Hz, 1H), 5.37-5.27 (m, 1H), 5.25 (t, J=7.5 Hz, 1H), 5.12-5.03 (m, 1H), 2.83-2.70 (m, 1H), 2.48-2.42 (m, 1H), 2.36-2.28 (m, 1H), 2.27-2.19 (m, 1H), 2.16-1.98 (m, 3H), 1.93-1.69 (m, 2H), 1.64-1.54 (m, 1H), 1.46-1.37 (m, 4H), 1.37-1.22 (m, 7H), 1.07 (d, J=7.0 Hz, 3H). MS (ESI) m/z: [M+H]⁺ Found 583.2.

### Example 136

### N—((S)-(4,4-Difluorocyclohexyl)(6-((R)-1-((R*)-4,4,4-trifluoro-3-methylbutanamido)ethyl)-1H-benzo[d]imidazol-2-yl)methyl)-2,5-difluorobenzamide

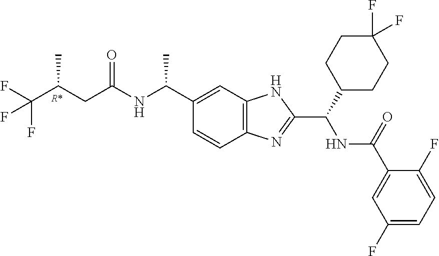 [SMILES: C[C@H](CC(=O)N[C@H](C)c1ccc2nc([C@@H](NC(=O)c3cc(F)ccc3F)C3CCC(F)(F)CC3)[nH]c2c1)C(F)(F)F]

The title compound was prepared as described in Example 121, using (R*)—N—((R)-1-(2-((S)-amino(4,4-difluorocyclohexyl)methyl)-1H-benzo[d]imidazol-5-yl)ethyl)-4,4,4-trifluoro-3-methylbutanamide (Intermediate 146) in place of N—((R)-1-(2-((S)-amino(4,4-difluorocyclohexyl)methyl)-1H-benzo[d]imidazol-5-yl)ethyl)-4,4,4-trifluorobutanamide and 2,5-difluorobenzoic acid in place of 1-(3,3,3-trifluoropropyl)-1H-pyrazole-5-carboxylic acid to afford the title compound as a white powder. ¹H NMR (400 MHz, DMSO-d₆, benzimidazole NH absent from exchange) δ 9.18-9.12 (m, 1H), 8.58 (d, J=7.6 Hz, 1H), 7.65 (d, J=8.4 Hz, 1H), 7.58 (s, 1H), 7.56-7.51 (m, 1H), 7.48-7.33 (m, 3H), 5.26 (t, J=7.4 Hz, 1H), 5.12-5.03 (m, 1H), 2.83-2.70 (m, 1H), 2.43 (d, J=4.6 Hz, 1H), 2.31-2.18 (m, 2H), 2.14-1.96 (m, 3H), 1.93-1.70 (m, 2H), 1.64-1.54 (m, 1H), 1.49-1.27 (m, 5H), 1.07 (d, J=7.0 Hz, 3H). MS (ESI) m/z: [M+H]⁺ Found 587.2.

### Example 137

### N—((S)-(4,4-Difluorocyclohexyl)(6-((R)-1-((S*)-4,4,4-trifluoro-3-methylbutanamido)ethyl)-1H-benzo[d]imidazol-2-yl)methyl)-1-(2,2,2-trifluoroethyl)-1H-pyrazole-5-carboxamide

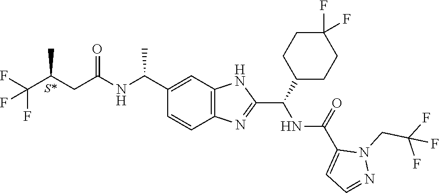 [SMILES: C[C@@H](NC(=O)C[C@H](C)C(F)(F)F)c1ccc2nc([C@@H](NC(=O)c3ccnn3CC(F)(F)F)C3CCC(F)(F)CC3)[nH]c2c1]

The title compound was prepared as described in Example 121, using (S*)—N—((R)-1-(2-((S)-amino(4,4-difluorocyclohexyl)methyl)-1H-benzo[d]imidazol-5-yl)ethyl)-4,4,4-trifluoro-3-methylbutanamide (Intermediate 145) in place of N—((R)-1-(2-((S)-amino(4,4-difluorocyclohexyl)methyl)-1H-benzo[d]imidazol-5-yl)ethyl)-4,4,4-trifluorobutanamide and 1-(2,2,2-trifluoroethyl)-1H-pyrazole-5-carboxylic acid in place of 1-(3,3,3-trifluoropropyl)-1H-pyrazole-5-carboxylic acid to afford the title compound as a white powder. ¹H NMR (400 MHz, DMSO-d₆, benzimidazole NH absent from exchange) δ 9.27 (d, J=7.4 Hz, 1H), 8.60 (d, J=7.8 Hz, 1H), 7.73 (d, J=2.0 Hz, 1H), 7.65 (d, J=8.5 Hz, 1H), 7.58 (s, 1H), 7.39-7.32 (m, 1H), 7.25 (d, J=2.0 Hz, 1H), 5.57-5.32 (m, 2H), 5.25 (t, J=7.7 Hz, 1H), 5.11-5.02 (m, 1H), 2.82-2.69 (m, 1H), 2.49-2.44 (m, 1H), 2.38-2.25 (m, 1H), 2.24-2.16 (m, 1H), 2.14-1.95 (m, 3H), 1.92-1.70 (m, 2H), 1.64-1.53 (m, 1H), 1.45-1.33 (m, 4H), 1.35-1.21 (m, 1H), 0.98 (d, J=6.9 Hz, 3H). MS (ESI) m/z: [M+H]⁺ Found 623.3.

### Example 138

### N—((S)-(4,4-Difluorocyclohexyl)(6-((R)-1-((S*)-4,4,4-trifluoro-3-methylbutanamido)ethyl)-1H-benzo[d]imidazol-2-yl)methyl)-1-(3,3,3-trifluoropropyl)-1H-pyrazole-5-carboxamide

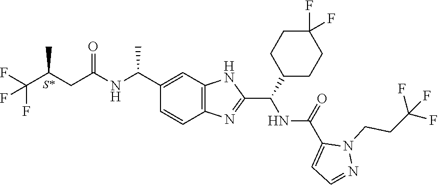 [SMILES: C[C@@H](NC(=O)C[C@H](C)C(F)(F)F)c1ccc2nc([C@@H](NC(=O)c3ccnn3CCC(F)(F)F)C3CCC(F)(F)CC3)[nH]c2c1]

The title compound was prepared as described in Example 121, using (S*)—N—((R)-1-(2-((S)-amino(4,4-difluorocyclohexyl)methyl)-1H-benzo[d]imidazol-5-yl)ethyl)-4,4,4-trifluoro-3-methylbutanamide (Intermediate 145) in place of N—((R)-1-(2-((S)-amino(4,4-difluorocyclohexyl)methyl)-1H-benzo[d]imidazol-5-yl)ethyl)-4,4,4-trifluorobutanamide to afford the title compound as a white powder. ¹H NMR (400 MHz, DMSO-d₆ benzimidazole NH absent from exchange) δ 9.12 (d, J=7.4 Hz, 1H), 8.60 (d, J=7.8 Hz, 1H), 7.65 (d, J=8.4 Hz, 1H), 7.60 (d, J=2.1 Hz, 1H), 7.58 (s, 1H), 7.39-7.32 (m, 1H), 7.14 (d, J=2.0 Hz, 1H), 5.25 (t, J=7.8 Hz, 1H), 5.12-5.00 (m, 1H), 4.79-4.62 (m, 2H), 2.85-2.70 (m, 3H), 2.48-2.45 (m, 1H), 2.37-2.26 (m, 1H), 2.24-2.15 (m, 1H), 2.13-1.97 (m, 3H), 1.92-1.70 (m, 2H), 1.63-1.55 (m, 1H), 1.47-1.35 (m, 4H), 1.35-1.21 (m, 1H), 0.98 (d, J=6.9 Hz, 3H). MS (ESI) m/z: [M+H]⁺ Found 637.3.

### Example 139

### N—((S)-(4,4-Difluorocyclohexyl)(6-((R)-1-((S*)-4,4,4-trifluoro-3-methylbutanamido)ethyl)-1H-benzo[d]imidazol-2-yl)methyl)-1-isopropyl-1H-pyrazole-5-carboxamide

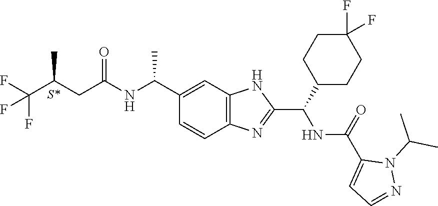 [SMILES: CC(C)n1nccc1C(=O)N[C@H](c1nc2ccc([C@@H](C)NC(=O)C[C@H](C)C(F)(F)F)cc2[nH]1)C1CCC(F)(F)CC1]

The title compound was prepared as described in Example 121, using (S*)—N—((R)-1-(2-((S)-amino(4,4-difluorocyclohexyl)methyl)-1H-benzo[d]imidazol-5-yl)ethyl)-4,4,4-trifluoro-3-methylbutanamide (Intermediate 145) in place of N—((R)-1-(2-((S)-amino(4,4-difluorocyclohexyl)methyl)-1H-benzo[d]imidazol-5-yl)ethyl)-4,4,4-trifluorobutanamide and 1-isopropyl-1H-pyrazole-5-carboxylic acid in place of 1-(3,3,3-trifluoropropyl)-1H-pyrazole-5-carboxylic acid to afford the title compound as a white powder. ¹H NMR (400 MHz, DMSO-d₆ benzimidazole NH absent from exchange) δ 9.06-8.99 (m, 1H), 8.61 (d, J=7.6 Hz, 1H), 7.66 (d, J=8.4 Hz, 1H), 7.59 (s, 1H), 7.55 (d, J=1.9 Hz, 1H), 7.41-7.33 (m, 1H), 7.03 (d, J=2.0 Hz, 1H), 5.38-5.26 (m, 1H), 5.23 (t, J=7.5 Hz, 1H), 5.12-5.01 (m, 1H), 2.82-2.70 (m, 1H), 2.48-2.45 (m, 1H), 2.36-2.25 (m, 1H), 2.25-2.15 (m, 1H), 2.15-1.97 (m, 3H), 1.94-1.69 (m, 2H), 1.64-1.54 (m, 1H), 1.46-1.22 (m, 11H), 0.98 (d, J=6.9 Hz, 3H). MS (ESI) m/z: [M+H]⁺ Found 583.3.

### Example 140

### N—((S)-(4,4-Difluorocyclohexyl)(6-((R)-1-((S*)-4,4,4-trifluoro-3-methylbutanamido)ethyl)-1H-benzo[d]imidazol-2-yl)methyl)-2,5-difluorobenzamide

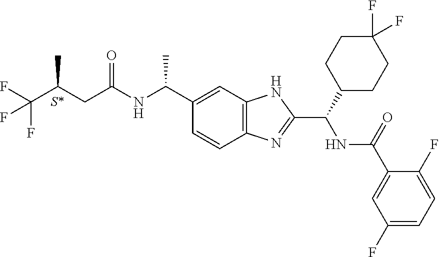 [SMILES: C[C@@H](NC(=O)C[C@H](C)C(F)(F)F)c1ccc2nc([C@@H](NC(=O)c3cc(F)ccc3F)C3CCC(F)(F)CC3)[nH]c2c1]

The title compound was prepared as described in Example 121, using (S*)—N—((R)-1-(2-((S)-amino(4,4-difluorocyclohexyl)methyl)-1H-benzo[d]imidazol-5-yl)ethyl)-4,4,4-trifluoro-3-methylbutanamide (Intermediate 145) in place of N—((R)-1-(2-((S)-amino(4,4-difluorocyclohexyl)methyl)-1H-benzo[d]imidazol-5-yl)ethyl)-4,4,4-trifluorobutanamide and 2,5-difluorobenzoic acid in place of 1-(3,3,3-trifluoropropyl)-1H-pyrazole-5-carboxylic acid to afford the title compound as a white powder. ¹H NMR (400 MHz, DMSO-d₆, benzimidazole NH absent from exchange) δ 9.19-9.11 (m, 1H), 8.60 (d, J=7.8 Hz, 1H), 7.66 (d, J=8.4 Hz, 1H), 7.60 (s, 1H), 7.56-7.50 (m, 1H), 7.48-7.34 (m, 3H), 5.27 (t, J=7.4 Hz, 1H), 5.12-5.03 (m, 1H), 2.82-2.67 (m, 1H), 2.49-2.45 (m, 1H), 2.32-2.17 (m, 2H), 2.14-1.95 (m, 3H), 1.92-1.70 (m, 2H), 1.63-1.55 (m, 1H), 1.48-1.27 (m, 5H), 0.99 (d, J=7.0 Hz, 3H). MS (ESI) m/z: [M+H]⁺ Found 587.2.

### Example 141

### N—((S)-(5-((R)-Cyclopropyl(2-(3,3-difluorocyclobutyl)acetamido)methyl)-1H-benzo[d]imidazol-2-yl)(4,4-difluorocyclohexyl)methyl)-4-methyl-1,2,5-oxadiazole-3-carboxamide

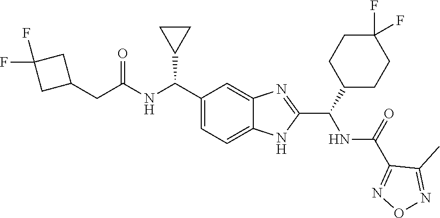 [SMILES: Cc1nonc1C(=O)N[C@H](c1nc2cc([C@H](NC(=O)CC3CC(F)(F)C3)C3CC3)ccc2[nH]1)C1CCC(F)(F)CC1]

To a stirred solution of 4-methyl-1,2,5-oxadiazole-3-carboxylic acid (76.4 mg, 59.6 mmol) and 1-propanephosphonic anhydride (0.32 mL, 53.7 mmol, 50% in EtOAc) in EtOAc (1.49 mL) was added N,N-diisopropylethylamine (0.31 mL, 1.79 mmol). After 3 min, N—((R)-(2-((S)-amino(4,4-difluorocyclohexyl)methyl)-1H-benzo[d]imidazol-6-yl)(cyclopropyl)methyl)-2-(3,3-difluorocyclobutyl)acetamide hydrochloride (150 mg, 29.8 mmol, Intermediate 159) was added. After 4 h at rt, additional portions of 4-methyl-1,2,5-oxadiazole-3-carboxylic acid (76.4 mg, 59.6 mmol), 1-propanephosphonic anhydride (0.32 mL, 53.7 mmol, 50% in EtOAc) and N,N-diisopropylethylamine (0.31 mL, 1.79 mmol) were added. After stirring for an additional 2 h at rt, additional portions of 4-methyl-1,2,5-oxadiazole-3-carboxylic acid (76.4 mg, 59.6 mmol), 1-propanephosphonic anhydride (0.32 mL, 53.7 mmol, 50% in EtOAc) and N,N-diisopropylethylamine (0.31 mL, 1.79 mmol) were added to reach full consumption of the amine after stirring at rt for another 1.5 h. The reaction mixture was diluted with water (15 mL) and the aqueous layer was extracted with EtOAc (3×5 mL). The combined organics were washed with saturated aqueous NaHCO₃ (10 mL), concentrated, dissolved in DMSO (3.5 mL) and purified by preparative HPLC (Waters XSelect CSH C18, 5 μm, 19×100 mm, 20-55% ACN in water with 0.16% TFA) to give the title compound as a white solid. ¹H NMR (500 MHz, DMSO-d₆) δ 9.78 (br d, J=7.5 Hz, 1H), 8.57 (d, J=8.1 Hz, 1H), 7.64 (d, J=8.4 Hz, 1H), 7.61 (s, 1H), 7.38 (br d, J=8.3 Hz, 1H), 5.30 (t, J=7.7 Hz, 1H), 4.33 (t, J=8.5 Hz, 1H), 2.69-2.56 (m, 2H), 2.47 (s, 3H), 2.40-2.22 (m, 6H), 2.13-1.96 (m, 3H), 1.91-1.71 (m, 2H), 1.61 (br d, J=12.4 Hz, 1H), 1.47-1.37 (m, 1H), 1.31 (dq, J=3.6, 12.6 Hz, 1H), 1.20-1.12 (m, 1H), 0.56-0.50 (m, 1H), 0.50-0.44 (m, 1H), 0.39-0.31 (m, 2H). MS (ESI) m/z: [M+H]⁺ Found 577.6.

### Example 142

### N—((S)-(5-((R)-Cyclopropyl(2-(3,3-difluorocyclobutyl)acetamido)methyl)-1H-benzo[d]imidazol-2-yl)(4,4-difluorocyclohexyl)methyl)-1-isopropyl-1H-pyrazole-5-carboxamide

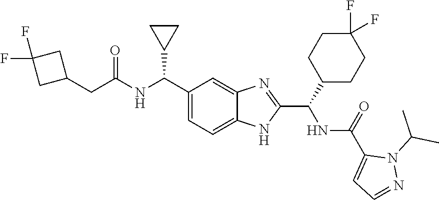 [SMILES: CC(C)n1nccc1C(=O)N[C@H](c1nc2cc([C@H](NC(=O)CC3CC(F)(F)C3)C3CC3)ccc2[nH]1)C1CCC(F)(F)CC1]

The title compound was prepared as described in Example 141, using 1-isopropyl-1H-pyrazole-5-carboxylic acid in place of 4-methyl-1,2,5-oxadiazole-3-carboxylic acid to provide the title compound as a white solid. ¹H NMR (500 MHz, DMSO-d₆) δ 9.03 (br d, J=7.1 Hz, 1H), 8.58 (d, J=8.1 Hz, 1H), 7.65 (d, J=8.5 Hz, 1H), 7.62 (s, 1H), 7.54 (d, J=1.9 Hz, 1H), 7.39 (br d, J=8.4 Hz, 1H), 7.02 (d, J=2.0 Hz, 1H), 5.40-5.25 (m 1H), 5.23 (t, J=7.6 Hz, 1H), 4.33 (t, J=8.5 Hz, 1H), 2.70-2.55 (m, 2H), 2.41-2.21 (m, 6H), 2.14-1.97 (m, 3H), 1.90-1.70 (m, 2H), 1.59 (br d, J=12.4 Hz, 1H), 1.45-1.37 (m, 1H), 1.35 (d, J=6.5 Hz, 3H), 1.32 (d, J=6.6 Hz, 3H), 1.30-1.23 (m, 1H), 1.21-1.11 (m, 1H), 0.57-0.50 (m, 1H), 0.50-0.43 (m, 1H), 0.41-0.29 (m, 2H). MS (ESI) m/z: [M+H]⁺ Found 603.7.

### Example 143

### 4-Cyclopropyl-N—((S)-(5-((R)-cyclopropyl(2-(3,3-difluorocyclobutyl)acetamido)methyl)-1H-benzo[d]imidazol-2-yl)(4,4-difluorocyclohexyl)methyl)-1,2,5-oxadiazole-3-carboxamide

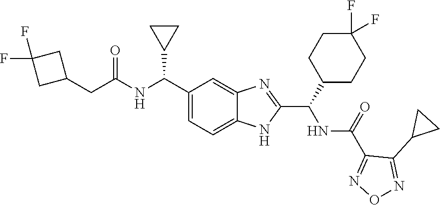 [SMILES: O=C(CC1CC(F)(F)C1)N[C@@H](c1ccc2[nH]c([C@@H](NC(=O)c3nonc3C3CC3)C3CCC(F)(F)CC3)nc2c1)C1CC1]

The title compound was prepared as described in Example 141, using 4-cyclopropyl-1,2,5-oxadiazole-3-carboxylic acid (Intermediate 78) in place of 4-methyl-1,2,5-oxadiazole-3-carboxylic acid to provide the title compound as a white solid. ¹H NMR (500 MHz, DMSO-d₆) δ 9.85 (d, J=7.5 Hz, 1H), 8.58 (d, J=8.1 Hz, 1H), 7.66 (d, J=8.5 Hz, 1H), 7.63 (s, 1H), 7.39 (dd, J=1.1, 8.4 Hz, 1H), 5.34 (t, J=7.7 Hz, 1H), 4.33 (t, J=8.5 Hz, 1H), 2.70-2.56 (m, 2H), 2.41-2.23 (m, 7H), 2.14-1.94 (m, 3H), 1.91-1.71 (m, 2H), 1.61 (br d, J=12.5 Hz, 1H), 1.48-1.35 (m, 1H), 1.35-1.27 (m, 1H), 1.21-1.07 (m, 3H), 0.99-0.95 (m, 2H), 0.57-0.50 (m, 1H), 0.50-0.44 (m, 1H), 0.40-0.30 (m, 2H). MS (ESI) m/z: [M+H]⁺ Found 603.7.

### Example 144

### N—((S)-(5-((R)-Cyclopropyl(2-(3,3-difluorocyclobutyl)acetamido)methyl)-1H-benzo[d]imidazol-2-yl)(4,4-difluorocyclohexyl)methyl)-4-(2,2-difluoroethoxy)-1,2,5-oxadiazole-3-carboxamide

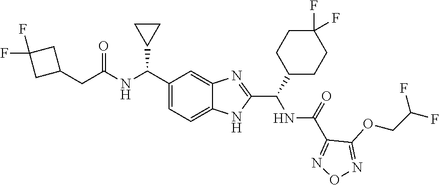 [SMILES: O=C(CC1CC(F)(F)C1)N[C@@H](c1ccc2[nH]c([C@@H](NC(=O)c3nonc3OCC(F)F)C3CCC(F)(F)CC3)nc2c1)C1CC1]

The title compound was prepared as described in Example 141, using 4-(2,2-difluoroethoxy)-1,2,5-oxadiazole-3-carboxylic acid (Intermediate 231) in place of 4-methyl-1,2,5-oxadiazole-3-carboxylic acid to provide the title compound as a white solid. ¹H NMR (500 MHz, DMSO-d₆) δ 12.39 (br s, 1H), 9.53 (br s, 1H), 8.44 (br s, 1H), 7.60-7.47 (m, 1H), 7.43 (br s, 1H), 7.17 (br d, J=8.1 Hz, 1H), 6.42 (tt, J=3.1, 53.7 Hz, 1H), 5.15 (br d, J=6.5 Hz, 1H), 4.70 (tt, J=2.7, 14.6 Hz, 2H), 4.34 (t, J=8.5 Hz, 1H), 2.70-2.55 (m, 2H), 2.41-2.17 (m, 6H), 2.10-1.92 (m, 3H), 1.88-1.68 (m, 2H), 1.54 (br d, J=12.5 Hz, 1H), 1.43-1.24 (m, 2H), 1.21-1.10 (m, 1H), 0.48 (quin, J=8.8 Hz, 2H), 0.38-0.24 (m, 2H). MS (ESI) m/z: [M+H]⁺ Found 643.2.

### Example 145

### N—((S)-(5-((S)-Cyclopropyl(2-(3,3-difluorocyclobutyl)acetamido)methyl)-1H-benzo[d]imidazol-2-yl)(4,4-difluorocyclohexyl)methyl)-4-methyl-1,2,5-oxadiazole-3-carboxamide

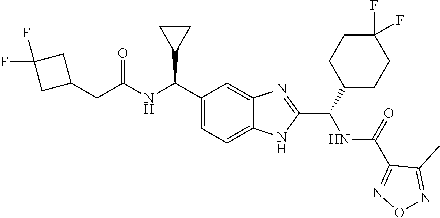 [SMILES: Cc1nonc1C(=O)N[C@H](c1nc2cc([C@@H](NC(=O)CC3CC(F)(F)C3)C3CC3)ccc2[nH]1)C1CCC(F)(F)CC1]

The title compound was prepared as described in Example 141, using N—((S)-(2-((S)-amino(4,4-difluorocyclohexyl)methyl)-1H-benzo[d]imidazol-6-yl)(cyclopropyl)methyl)-2-(3,3-difluorocyclobutyl)acetamide hydrochloride (Intermediate 161) in place of N—((R)-(2-((S)-amino(4,4-difluorocyclohexyl)methyl)-1H-benzo[d]imidazol-6-yl)(cyclopropyl)methyl)-2-(3,3-difluorocyclobutyl)acetamide hydrochloride to provide the title compound as a white solid. ¹H NMR (500 MHz, DMSO-d₆) δ 9.80 (d, J=7.5 Hz, 1H), 8.58 (d, J=8.1 Hz, 1H), 7.65 (d, J=8.5 Hz, 1H), 7.62 (s, 1H), 7.39 (dd, J=0.9, 8.4 Hz, 1H), 5.31 (t, J=7.8 Hz, 1H), 4.33 (t, J=8.5 Hz, 1H), 2.70-2.56 (m, 2H), 2.47 (s, 3H), 2.41-2.21 (m, 6H), 2.14-1.95 (m, 3H), 1.91-1.71 (m, 2H), 1.61 (br d, J=12.3 Hz, 1H), 1.49-1.37 (m, 1H), 1.36-1.25 (m, 1H), 1.23-1.12 (m, 1H), 0.57-0.51 (m, 1H), 0.50-0.44 (m, 1H), 0.40-0.30 (m, 2H). MS (ESI) m/z: [M+H]⁺ Found 577.7.

### Example 146

### N—((S)-(5-((S)-Cyclopropyl(2-(3,3-difluorocyclobutyl)acetamido)methyl)-1H-benzo[d]imidazol-2-yl)(4,4-difluorocyclohexyl)methyl)-1-isopropyl-1H-pyrazole-5-carboxamide

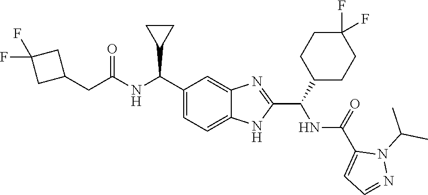 [SMILES: CC(C)n1nccc1C(=O)N[C@H](c1nc2cc([C@@H](NC(=O)CC3CC(F)(F)C3)C3CC3)ccc2[nH]1)C1CCC(F)(F)CC1]

The title compound was prepared as described in Example 141, using N—((S)-(2-((S)-amino(4,4-difluorocyclohexyl)methyl)-1H-benzo[d]imidazol-6-yl)(cyclopropyl)methyl)-2-(3,3-difluorocyclobutyl)acetamide hydrochloride (Intermediate 161) in place of N—((R)-(2-((S)-amino(4,4-difluorocyclohexyl)methyl)-1H-benzo[d]imidazol-6-yl)(cyclopropyl)methyl)-2-(3,3-difluorocyclobutyl)acetamide hydrochloride and 1-isopropyl-1H-pyrazole-5-carboxylic acid in place of 4-methyl-1,2,5-oxadiazole-3-carboxylic acid to provide the title compound as a white solid. ¹H NMR (500 MHz, DMSO-d₆) δ 9.08 (d, J=7.0 Hz, 1H), 8.59 (d, J=8.1 Hz, 1H), 7.67 (d, J=8.5 Hz, 1H), 7.64 (s, 1H), 7.54 (d, J=2.0 Hz, 1H), 7.42 (br d, J=8.5 Hz, 1H), 7.02 (d, J=2.0 Hz, 1H), 5.37-5.25 (m, 1H), 5.25 (t, J=7.6 Hz, 1H), 4.33 (t, J=8.5 Hz, 1H), 2.71-2.55 (m, 2H), 2.42-2.22 (m, 6H), 2.13-1.97 (m, 3H), 1.91-1.70 (m, 2H), 1.59 (br d, J=12.6 Hz, 1H), 1.46-1.37 (m, 1H), 1.34 (d, J=6.6 Hz, 3H), 1.32 (d, J=6.6 Hz, 3H), 1.30-1.24 (m, 1H), 1.22-1.12 (m, 1H), 0.57-0.51 (m, 1H), 0.50-0.44 (m, 1H), 0.40-0.25 (m, 2H). MS (ESI) m/z: [M+H]⁺ Found 603.7.

### Example 147

### 4-Cyclopropyl-N—((S)-(5-((R)-cyclopropyl(3-cyclopropyl-2,2-difluoropropanamido)methyl)-1H-benzo[d]imidazol-2-yl)(4,4-difluorocyclohexyl)methyl)-1,2,5-oxadiazole-3-carboxamide

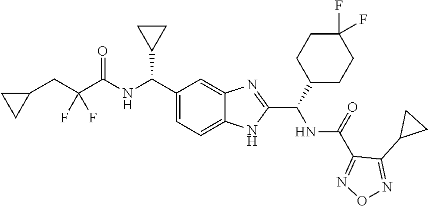 [SMILES: O=C(N[C@H](c1nc2cc([C@H](NC(=O)C(F)(F)CC3CC3)C3CC3)ccc2[nH]1)C1CCC(F)(F)CC1)c1nonc1C1CC1]

The title compound was prepared as described in Example 141, using N—((R)-(2-((S)-amino(4,4-difluorocyclohexyl)methyl)-1H-benzo[d]imidazol-5-yl)(cyclopropyl)methyl)-3-cyclopropyl-2,2-difluoropropanamide (Intermediate 164) in place of N—((R)-(2-((S)-amino(4,4-difluorocyclohexyl)methyl)-1H-benzo[d]imidazol-6-yl)(cyclopropyl)methyl)-2-(3,3-difluorocyclobutyl)acetamide hydrochloride and 4-cyclopropyl-1,2,5-oxadiazole-3-carboxylic acid (Intermediate 78) in place of 4-methyl-1,2,5-oxadiazole-3-carboxylic acid to provide the title compound as a white solid. ¹H NMR (500 MHz, DMSO-d₆) δ 9.79 (br d, J=7.6 Hz, 1H), 9.39 (br d, J=8.4 Hz, 1H), 7.69 (s, 1H), 7.64 (d, J=8.4 Hz, 1H), 7.44 (br d, J=8.3 Hz, 1H), 5.31 (t, J=7.8 Hz, 1H), 4.25 (t, J=9.0 Hz, 1H), 2.36-2.28 (m, 2H), 2.13-2.05 (m, 2H), 2.04-1.91 (m, 4H), 1.90-1.71 (m, 2H), 1.61 (br d, J=12.6 Hz, 1H), 1.50-1.37 (m, 2H), 1.37-1.26 (m, 1H), 1.16-1.08 (m, 2H), 1.00-0.95 (m, 2H), 0.72-0.63 (m, 1H), 0.62-0.55 (m, 1H), 0.54-0.48 (m, 1H), 0.46-0.39 (m, 2H), 0.38-0.31 (m, 2H), 0.15-0.03 (m, 2H). MS (ESI) m/z: [M+H]⁺ Found 603.3.

### Example 148

### N—((S)-1-(6-((R)-Cyclopropyl(2-(3,3-difluorocyclobutyl)acetamido)methyl)-1H-benzo[d]imidazol-2-yl)-2-(6,6-difluorospiro[3.3]heptan-2-yl)ethyl)-1-isopropyl-1H-pyrazole-5-carboxamide

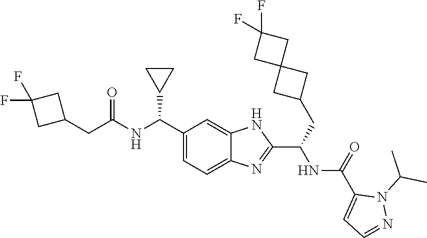 [SMILES: CC(C)n1nccc1C(=O)N[C@@H](CC1CC2(C1)CC(F)(F)C2)c1nc2ccc([C@H](NC(=O)CC3CC(F)(F)C3)C3CC3)cc2[nH]1]

The title compound was prepared as described in Example 141, using N—((R)-(2-((S)-1-amino-2-(6,6-difluorospiro[3.3]heptan-2-yl)ethyl)-1H-benzo[d]imidazol-6-yl)(cyclopropyl)methyl)-2-(3,3-difluorocyclobutyl)acetamide (Intermediate 173) in place of N—((R)-(2-((S)-amino(4,4-difluorocyclohexyl)methyl)-1H-benzo[d]imidazol-6-yl)(cyclopropyl)methyl)-2-(3,3-difluorocyclobutyl)acetamide hydrochloride and 1-isopropyl-1H-pyrazole-5-carboxylic acid in place of 4-methyl-1,2,5-oxadiazole-3-carboxylic acid to provide the title compound as a white solid. ¹H NMR (500 MHz, DMSO-d₆) δ 9.07 (d, J=7.0 Hz, 1H), 8.63 (d, J=8.1 Hz, 1H), 7.69 (d, J=8.5 Hz, 1H), 7.64 (s, 1H), 7.55 (d, J=2.0 Hz, 1H), 7.46 (br d, J=8.5 Hz, 1H), 6.98 (d, J=2.0 Hz, 1H), 5.38 (quin, J=6.6 Hz, 1H), 5.28 (q, J=7.3 Hz, 1H), 4.33 (t, J=8.5 Hz, 1H), 2.69-2.51 (m, 5H), 2.42-2.26 (m, 6H), 2.24-2.17 (m, 3H), 2.16-2.05 (m, 1H), 1.91-1.80 (m, 2H), 1.35 (d, J=6.6 Hz, 6H), 1.20-1.11 (m, 1H), 0.59-0.51 (m, 1H), 0.51-0.44 (m, 1H), 0.42-0.30 (m, 2H). MS (ESI) m/z: [M+H]⁺ Found 629.3.

### Example 149

### N—((R*)-1-(6-((R)-Cyclopropyl(2-(3,3-difluorocyclobutyl)acetamido)methyl)-1H-benzo[d]imidazol-2-yl)-2-((3,3-difluoro-2-methylbutan-2-yl)oxy)ethyl)-1-isopropyl-1H-pyrazole-5-carboxamide

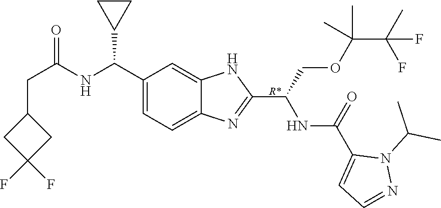 [SMILES: CC(C)n1nccc1C(=O)N[C@@H](COC(C)(C)C(C)(F)F)c1nc2ccc([C@H](NC(=O)CC3CC(F)(F)C3)C3CC3)cc2[nH]1]

To a solution of 1-isopropyl-1H-pyrazole-5-carboxylic acid (53 mg, 0.344 mmol) and DIPEA (0.11 mL, 0.645 mmol) in EtOAc (1 mL) was added T3P©(0.21 mL, 0.344 mmol, 50% in EtOAc). The mixture was stirred at rt for 3 min then a solution of N—((R)-(2-((R*)-1-amino-2-((3,3-difluoro-2-methylbutan-2-yl)oxy)ethyl)-1H-benzo[d]imidazol-5-yl)(cyclopropyl)methyl)-2-(3,3-difluorocyclobutyl)acetamide (104 mg, 0.215 mmol, Intermediate 193) in EtOAc (1 mL) was added. The mixture was stirred for 30 min at rt then T3P© (0.1 mL, 0.172 mmol, 50% in EtOAc) was added and the reaction stirred for an additional 30 min at rt. The mixture was diluted with EtOAc and washed with saturated aqueous ammonium chloride, saturated aqueous sodium bicarbonate, and brine. Then, the organic layer was dried over anhydrous Na₂SO₄, filtered and condensed. Purification by silica gel chromatography (10-80% (10% MeOH/EtOAc)/hexanes) provided the title compound. ¹H NMR (500 MHz, DMSO-d₆) δ 12.31 (br d, J=4.4 Hz, 1H), 8.84 (dd, J=8.4, 5.9 Hz, 1H), 8.45 (dd, J=17.3, 8.6 Hz, 1H), 7.56-7.48 (m, 2H), 7.43-7.36 (m, 1H), 7.16 (t, J=8.6 Hz, 1H), 6.92 (d, J=2.0 Hz, 1H), 5.52-5.38 (m, 2H), 4.39-4.32 (m, 1H), 4.13-4.04 (m, 1H), 3.94-3.83 (m, 1H), 2.70-2.54 (m, 2H), 2.43-2.21 (m, 5H), 1.49 (t, J=19.5 Hz, 3H), 1.37 (d, J=6.6 Hz, 6H), 1.23-1.19 (m, 6H), 1.19-1.12 (m, 1H), 0.53-0.43 (m, 2H), 0.35-0.27 (m, 2H). MS (ESI) m/z: [M+H]⁺ Found 621.3.

### Example 150

### N—((R*)-1-(6-((R)-Cyclopropyl(2-(3,3-difluorocyclobutyl)acetamido)methyl)-1H-benzo[d]imidazol-2-yl)-2-(((R*)-1,1,1-trifluoropropan-2-yl)oxy)ethyl)-4-methyl-1,2,5-oxadiazole-3-carboxamide

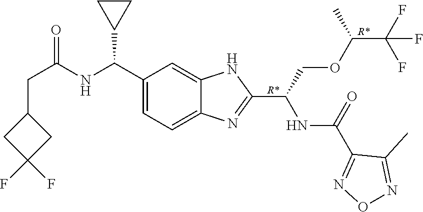 [SMILES: Cc1nonc1C(=O)N[C@@H](CO[C@H](C)C(F)(F)F)c1nc2ccc([C@H](NC(=O)CC3CC(F)(F)C3)C3CC3)cc2[nH]1]

### Example 151

### N—((R*)-1-(6-((R)-Cyclopropyl(2-(3,3-difluorocyclobutyl)acetamido)methyl)-1H-benzo[d]imidazol-2-yl)-2-(((S*)-1,1,1-trifluoropropan-2-yl)oxy)ethyl)-4-methyl-1,2,5-oxadiazole-3-carboxamide

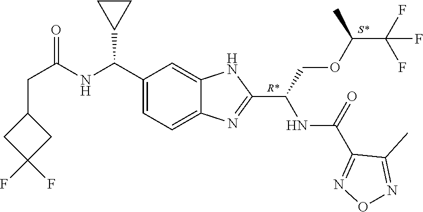 [SMILES: Cc1nonc1C(=O)N[C@@H](CO[C@@H](C)C(F)(F)F)c1nc2ccc([C@H](NC(=O)CC3CC(F)(F)C3)C3CC3)cc2[nH]1]

A solution of N-((1R)-(2-((1R*)-1-amino-2-((1,1,1-trifluoropropan-2-yl)oxy)ethyl)-1H-benzo[d]imidazol-6-yl)(cyclopropyl)methyl)-2-(3,3-difluorocyclobutyl)acetamide (58 mg, 0.123 mmol, Intermediate 189), 4-methyl-1,2,5-oxadiazole-3-carboxylic acid (19 mg, 0.148 mmol), DIPEA (0.035 mL, 0.197 mmol) and HOBt (20 mg, 0.148 mmol) in MeCN (2 mL) was heated to 45° C. and then EDCI (28 mg, 0.148 mmol) was added. The reaction was stirred at 45° C. for 60 min then quenched with H₂O and condensed. The residue was dissolved in EtOAc and washed with saturated aqueous ammonium chloride, saturated aqueous sodium bicarbonate and brine. Then the organic layer was dried over anhydrous Na₂SO₄, filtered and condensed. Purification by silica gel chromatography (5-100% (10% MeOH/EtOAc)/hexanes) afforded a mixture of diastereomers. The diastereomers were separated by SFC using a chiral stationary phase (Chiralpak IA, 5 μm, 250×21 mm, Mobile phase: 20% methanol with 0.2% TEA, 80% CO₂). The first eluting isomer was Example 150 and the second eluting isomer was Example 151. Example 150: ¹H NMR (500 MHz, DMSO-d₆) δ 12.33 (s, 1H), 9.71-9.58 (m, 1H), 8.46 (dd, J=16.8, 8.6 Hz, 1H), 7.60-7.49 (m, 1H), 7.45-7.35 (m, 1H), 7.21-7.12 (m, 1H), 5.57-5.48 (m, 1H), 4.40-4.25 (m, 3H), 4.18-4.10 (m, 1H), 2.70-2.55 (m, 2H), 2.52 (s, 3H), 2.42-2.21 (m, 5H), 1.22 (br d, J=6.4 Hz, 3H), 1.19-1.10 (m, 1H), 0.55-0.42 (m, 2H), 0.36-0.27 (m, 2H). MS (ESI) m/z: [M+H]⁺ Found 585.2. Example 151: ¹H NMR (500 MHz, DMSO-d₆) δ 12.33 (s, 1H), 9.71-9.58 (m, 1H), 8.46 (dd, J=16.8, 8.6 Hz, 1H), 7.60-7.49 (m, 1H), 7.45-7.35 (m, 1H), 7.21-7.12 (m, 1H), 5.57-5.48 (m, 1H), 4.40-4.25 (m, 3H), 4.18-4.10 (m, 1H), 2.70-2.55 (m, 2H), 2.52 (s, 3H), 2.42-2.21 (m, 5H), 1.22 (br d, J=6.4 Hz, 3H), 1.19-1.10 (m, 1H), 0.55-0.42 (m, 2H), 0.36-0.27 (m, 2H). MS (ESI) m/z: [M+H]⁺ Found 585.2.

### Example 152

### 4-Cyclopropyl-N—((R*)-1-(6-((R)-1-(2-(3,3-difluorocyclobutyl)acetamido)ethyl)-1H-benzo[d]imidazol-2-yl)-2-((1,1,1-trifluoro-2-methylpropan-2-yl)oxy)ethyl)-1,2,5-oxadiazole-3-carboxamide

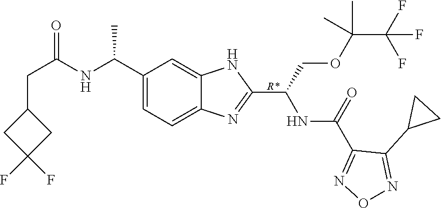 [SMILES: C[C@@H](NC(=O)CC1CC(F)(F)C1)c1ccc2nc([C@H](COC(C)(C)C(F)(F)F)NC(=O)c3nonc3C3CC3)[nH]c2c1]

A solution of N—((R)-1-(2-((R*)-1-amino-2-((1,1,1-trifluoro-2-methylpropan-2-yl)oxy)ethyl)-1H-benzo[d]imidazol-6-yl)ethyl)-2-(3,3-difluorocyclobutyl)acetamide (172 mg, 0.372 mmol, Intermediate 185), 4-cyclopropyl-1,2,5-oxadiazole-3-carboxylic acid (69 mg, 0.446 mmol), DIPEA (0.1 mL, 0.595 mmol) and HOBt (60 mg, 0.446 mmol) in MeCN (6 mL) was heated to 45° C. and then EDCI (86 mg, 0.446 mmol) was added. The reaction was stirred at 45° C. for 75 min then quenched with H₂O and condensed. The residue was dissolved in EtOAc and washed with saturated aqueous ammonium chloride, saturated aqueous sodium bicarbonate and brine. Then the organic layer was dried over anhydrous Na₂SO₄, filtered and condensed. Purification by silica gel chromatography (5-70% (10% MeOH/EtOAc)/hexanes) afforded a mixture of diastereomers. The diastereomers were separated by SFC using a chiral stationary phase (Chiralpak IA, 5 μm, 250×21 mm, Mobile phase: 25% methanol, 75% CO₂) and the major diastereomer was collected, frozen, and lyophilized to provide the title compound. ¹H NMR (500 MHz, DMSO-d₆) δ 12.37 (s, 1H), 9.58 (br t, J=8.4 Hz, 1H), 8.33 (br dd, J=18.1, 8.1 Hz, 1H), 7.58-7.47 (m, 1H), 7.43-7.33 (m, 1H), 7.12 (br dd, J=12.2, 8.6 Hz, 1H), 5.44 (td, J=8.3, 5.0 Hz, 1H), 5.00 (quin, J=7.1 Hz, 1H), 4.19 (dd, J=9.6, 4.8 Hz, 1H), 4.06-4.00 (m, 1H), 2.68-2.51 (m, 2H), 2.43-2.23 (m, 6H), 1.39-1.36 (m, 3H), 1.35-1.32 (m, 6H), 1.17-1.13 (m, 2H), 1.07-0.95 (m, 2H). MS (ESI) m/z: [M+H]⁺ Found 599.3.

### Example 153

### 4-Cyclopropyl-N-((1R*)-1-(6-((R)-cyclopropyl(2-(3,3-difluorocyclobutyl)acetamido)methyl)-1H-benzo[d]imidazol-2-yl)-2-((1,1,1-trifluoropropan-2-yl)oxy)ethyl)-1,2,5-oxadiazole-3-carboxamide

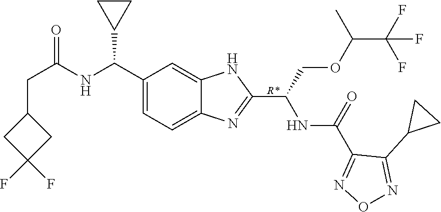 [SMILES: CC(OC[C@H](NC(=O)c1nonc1C1CC1)c1nc2ccc([C@H](NC(=O)CC3CC(F)(F)C3)C3CC3)cc2[nH]1)C(F)(F)F]

### Example 154

### 4-Cyclopropyl-N—((R*)-1-(6-((R)-cyclopropyl(2-(3,3-difluorocyclobutyl)acetamido)methyl)-1H-benzo[d]imidazol-2-yl)-2-(((S)-1,1,1-trifluoropropan-2-yl)oxy)ethyl)-1,2,5-oxadiazole-3-carboxamide

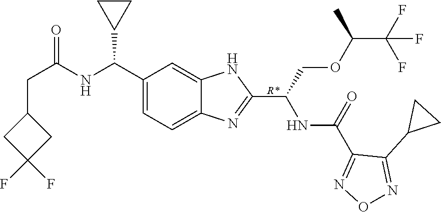 [SMILES: C[C@H](OC[C@H](NC(=O)c1nonc1C1CC1)c1nc2ccc([C@H](NC(=O)CC3CC(F)(F)C3)C3CC3)cc2[nH]1)C(F)(F)F]

### Example 155

### 4-Cyclopropyl-N—((R*)-1-(6-((R)-cyclopropyl(2-(3,3-difluorocyclobutyl)acetamido)methyl)-1H-benzo[d]imidazol-2-yl)-2-(((R)-1,1,1-trifluoropropan-2-yl)oxy)ethyl)-1,2,5-oxadiazole-3-carboxamide

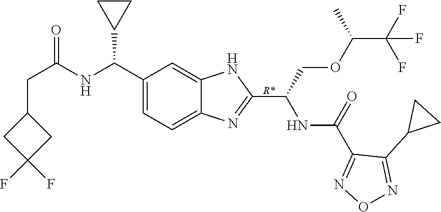 [SMILES: C[C@@H](OC[C@H](NC(=O)c1nonc1C1CC1)c1nc2ccc([C@H](NC(=O)CC3CC(F)(F)C3)C3CC3)cc2[nH]1)C(F)(F)F]

A solution of N-((1R)-(2-((1R*)-1-amino-2-((1,1,1-trifluoropropan-2-yl)oxy)ethyl)-1H-benzo[d]imidazol-6-yl)(cyclopropyl)methyl)-2-(3,3-difluorocyclobutyl)acetamide (175 mg, 0.369 mmol, Intermediate 189), 4-cyclopropyl-1,2,5-oxadiazole-3-carboxylic acid (68 mg, 0.443 mmol), DIPEA (0.1 mL, 0.59 mmol) and HOBt (60 mg, 0.443 mmol) in MeCN (6 mL) was heated to 45° C. and then EDCI (86 mg, 0.446 mmol) was added. The reaction was stirred at 45° C. for 75 min then quenched with H₂O and condensed. The residue was dissolved in EtOAc and washed with saturated aqueous ammonium chloride, saturated aqueous sodium bicarbonate and brine. Then the organic layer was dried over anhydrous Na₂SO₄, filtered and condensed. Purification by silica gel chromatography (5-60% (10% MeOH/EtOAc)/hexanes) afforded Example 153, a mixture of diastereomers. The diastereomers were separated by SFC using a chiral stationary phase (Chiralpak IG, 5 μm, 250×21 mm, Mobile phase: 20% methanol, 80% CO₂) to provide the title compounds. Example 155 was the first eluting isomer and Example 154 was the second eluting isomer. Example 153: ¹H NMR (500 MHz, DMSO-d₆) δ 12.37 (br s, 1H), 9.71-9.64 (m, 1H), 8.46 (dd, J=17.1, 8.6 Hz, 1H), 7.57-7.50 (m, 1H), 7.42-7.39 (m, 1H), 7.17 (t, J=7.8 Hz, 1H), 5.58-5.48 (m, 1H), 4.38-4.23 (m, 3H), 4.17-4.10 (m, 1H), 2.67-2.57 (m, 2H), 2.46-2.33 (m, 4H), 2.30 (br d, J=7.9 Hz, 2H), 1.26-1.21 (m, 3H), 1.18-1.14 (m, 3H), 1.07-0.97 (m, 2H), 0.54-0.42 (m, 2H), 0.36-0.27 (m, 2H). MS (ESI) m/z: [M+H]⁺ Found 611.3. Example 154: ¹H NMR (500 MHz, DMSO-d₆) δ 12.37 (br s, 1H), 9.71-9.64 (m, 1H), 8.46 (dd, J=17.1, 8.6 Hz, 1H), 7.57-7.50 (m, 1H), 7.42-7.39 (m, 1H), 7.17 (t, J=7.8 Hz, 1H), 5.58-5.48 (m, 1H), 4.38-4.23 (m, 3H), 4.17-4.10 (m, 1H), 2.67-2.57 (m, 2H), 2.46-2.33 (m, 4H), 2.30 (br d, J=7.9 Hz, 2H), 1.26-1.21 (m, 3H), 1.18-1.14 (m, 3H), 1.07-0.97 (m, 2H), 0.54-0.42 (m, 2H), 0.36-0.27 (m, 2H). MS (ESI) m/z: [M+H]⁺ Found 611.3. Example 155: ¹H NMR (500 MHz, DMSO-d₆) δ 12.37 (br s, 1H), 9.71-9.64 (m, 1H), 8.46 (dd, J=17.1, 8.6 Hz, 1H), 7.57-7.50 (m, 1H), 7.42-7.39 (m, 1H), 7.17 (t, J=7.8 Hz, 1H), 5.58-5.48 (m, 1H), 4.38-4.23 (m, 3H), 4.17-4.10 (m, 1H), 2.67-2.57 (m, 2H), 2.46-2.33 (m, 4H), 2.30 (br d, J=7.9 Hz, 2H), 1.26-1.21 (m, 3H), 1.18-1.14 (m, 3H), 1.07-0.97 (m, 2H), 0.54-0.42 (m, 2H), 0.36-0.27 (m, 2H). MS (ESI) m/z: [M+H]⁺ Found 611.3.

### Example 156

### N—((R*)-1-(6-((R)-Cyclopropyl(2-(3,3-difluorocyclobutyl)acetamido)methyl)-1H-benzo[d]imidazol-2-yl)-2-(((R)-1,1,1-trifluoropropan-2-yl)oxy)ethyl)-1-isopropyl-1H-pyrazole-5-carboxamide

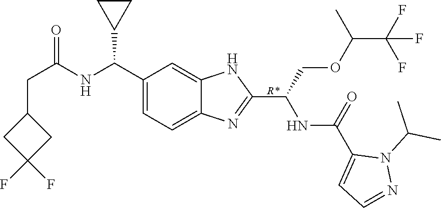 [SMILES: CC(C)n1nccc1C(=O)N[C@@H](COC(C)C(F)(F)F)c1nc2ccc([C@H](NC(=O)CC3CC(F)(F)C3)C3CC3)cc2[nH]1]

### Example 157

### N—((R*)-1-(6-((R)-Cyclopropyl(2-(3,3-difluorocyclobutyl)acetamido)methyl)-1H-benzo[d]imidazol-2-yl)-2-(((R)-1,1,1-trifluoropropan-2-yl)oxy)ethyl)-1-isopropyl-1H-pyrazole-5-carboxamide

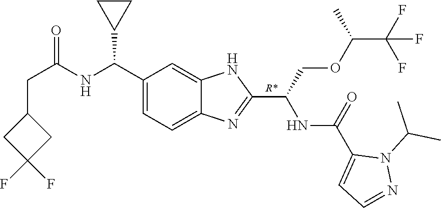 [SMILES: CC(C)n1nccc1C(=O)N[C@@H](CO[C@H](C)C(F)(F)F)c1nc2ccc([C@H](NC(=O)CC3CC(F)(F)C3)C3CC3)cc2[nH]1]

### Example 158

### N—((R*)-1-(6-((R)-Cyclopropyl(2-(3,3-difluorocyclobutyl)acetamido)methyl)-1H-benzo[d]imidazol-2-yl)-2-(((S)-1,1,1-trifluoropropan-2-yl)oxy)ethyl)-1-isopropyl-1H-pyrazole-5-carboxamide

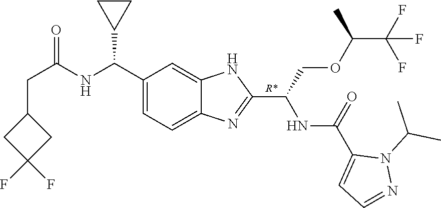 [SMILES: CC(C)n1nccc1C(=O)N[C@@H](CO[C@@H](C)C(F)(F)F)c1nc2ccc([C@H](NC(=O)CC3CC(F)(F)C3)C3CC3)cc2[nH]1]

A solution of N-((1R)-(2-((1R*)-1-amino-2-((1,1,1-trifluoropropan-2-yl)oxy)ethyl)-1H-benzo[d]imidazol-6-yl)(cyclopropyl)methyl)-2-(3,3-difluorocyclobutyl)acetamide (76 mg, 0.161 mmol, Intermediate 189), 1-isopropyl-1H-pyrazole-5-carboxylic acid (25 mg, 0.161 mmol), DIPEA (0.05 mL, 0.258 mmol) and HOBt (24 mg, 0.177 mmol) in MeCN (2 mL) was heated to 45° C. and then EDCI (86 mg, 0.446 mmol) was added. The reaction was stirred at 45° C. for 60 min then quenched with H₂O and evaporated under reduced pressure to remove volatiles. The residue was dissolved in EtOAc and washed with saturated aqueous ammonium chloride, saturated aqueous sodium bicarbonate and brine. Then the organic layer was dried over anhydrous Na₂SO₄, filtered, and evaporated to dryness. The crude material was purified by preparative HPLC (XBridge, C18, OBD, 50×100 mm, 5-95% MeCN/H₂O with 20 mM NH₄OH) to provide Example 156, a mixture of diastereomers. The diastereomers were separated by SFC using a chiral stationary phase (Whelk O1 SS, 5 μm, 250×21 mm, Mobile phase: 20% methanol, 80% CO₂) to afford the title compounds. Example 157 was the first eluting isomer and Example 158 was the second eluting isomer. Example 156: ¹H NMR (400 MHz, DMSO-d₆) δ 12.43-12.27 (m, 1H), 8.99-8.88 (m, 1H), 8.52-8.38 (m, 1H), 7.57-7.47 (m, 2H), 7.43-7.35 (m, 1H), 7.21-7.10 (m, 1H), 6.96-6.88 (m, 1H), 5.57-5.41 (m, 2H), 4.42-4.21 (m, 3H), 4.13-4.04 (m, 1H), 2.69-2.58 (m, 1H), 2.42-2.23 (m, 5H), 1.40-1.35 (m, 6H), 1.25-1.19 (m, 3H), 1.19-1.10 (m, 1H), 1.00-0.92 (m, 1H), 0.54-0.43 (m, 2H), 0.36-0.26 (m, 2H). MS (ESI) m/z: [M+H]⁺ Found 611.5. Example 157: ¹H NMR (400 MHz, DMSO-d₆) δ 12.43-12.27 (m, 1H), 8.99-8.88 (m, 1H), 8.52-8.38 (m, 1H), 7.57-7.47 (m, 2H), 7.43-7.35 (m, 1H), 7.21-7.10 (m, 1H), 6.96-6.88 (m, 1H), 5.57-5.41 (m, 2H), 4.42-4.21 (m, 3H), 4.13-4.04 (m, 1H), 2.69-2.58 (m, 1H), 2.42-2.23 (m, 5H), 1.40-1.35 (m, 6H), 1.25-1.19 (m, 3H), 1.19-1.10 (m, 1H), 1.00-0.92 (m, 1H), 0.54-0.43 (m, 2H), 0.36-0.26 (m, 2H). MS (ESI) m/z: [M+H]⁺ Found 611.5. Example 158: ¹H NMR (400 MHz, DMSO-d₆) δ 12.43-12.27 (m, 1H), 8.99-8.88 (m, 1H), 8.52-8.38 (m, 1H), 7.57-7.47 (m, 2H), 7.43-7.35 (m, 1H), 7.21-7.10 (m, 1H), 6.96-6.88 (m, 1H), 5.57-5.41 (m, 2H), 4.42-4.21 (m, 3H), 4.13-4.04 (m, 1H), 2.69-2.58 (m, 1H), 2.42-2.23 (m, 5H), 1.40-1.35 (m, 6H), 1.25-1.19 (m, 3H), 1.19-1.10 (m, 1H), 1.00-0.92 (m, 1H), 0.54-0.43 (m, 2H), 0.36-0.26 (m, 2H). MS (ESI) m/z: [M+H]⁺ Found 611.5.

### Example 159

### 4-Cyclopropyl-N—((R)-1-(6-((R)-cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-1,2,5-oxadiazole-3-carboxamide

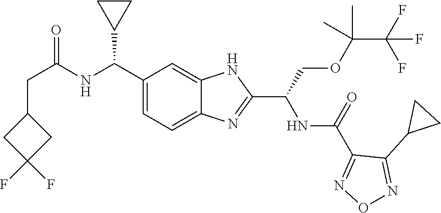 [SMILES: CC(C)(OC[C@H](NC(=O)c1nonc1C1CC1)c1nc2ccc([C@H](NC(=O)CC3CC(F)(F)C3)C3CC3)cc2[nH]1)C(F)(F)F]

The title compound was prepared as described for the synthesis of Example 149, using 4-cyclopropyl-1,2,5-oxadiazole-3-carboxylic acid (Intermediate 78) in place of 1-isopropyl-1H-pyrazole-5-carboxylic acid and N—((R)-(2-((R)-1-amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide (Intermediate 197) in place of N—((R)-(2-((R*)-1-amino-2-((3,3-difluoro-2-methylbutan-2-yl)oxy)ethyl)-1H-benzo[d]imidazol-5-yl)(cyclopropyl)methyl)-2-(3,3-difluorocyclobutyl)acetamide. ¹H NMR (600 MHz, DMSO-d₆) δ 12.37 (s, 1H), 9.58 (t, J=9.5 Hz, 1H), 8.50-8.42 (m, 1H), 7.52 (d, J=8.4 Hz, 1H), 7.44-7.39 (m, 1H), 7.17 (t, J=8.7 Hz, 1H), 5.45 (td, J=8.1, 5.3 Hz, 1H), 4.38-4.33 (m, 1H), 4.19 (dd, J=9.7, 4.8 Hz, 1H), 4.07-4.00 (m, 1H), 2.69-2.56 (m, 2H), 2.43-2.22 (m, 6H), 1.34 (d, J=3.4 Hz, 6H), 1.20-1.12 (m, 3H), 1.05-0.98 (m, 2H), 0.53-0.42 (m, 2H), 0.36-0.25 (m, 2H). MS (ESI) m/z: [M+H]⁺ Found 625.3.

### Example 160

### N—((R)-1-(5-((R)-Cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-1-isopropyl-1H-pyrazole-5-carboxamide

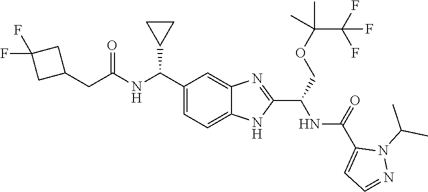 [SMILES: CC(C)n1nccc1C(=O)N[C@@H](COC(C)(C)C(F)(F)F)c1nc2cc([C@H](NC(=O)CC3CC(F)(F)C3)C3CC3)ccc2[nH]1]

A solution of N—((R)-(2-((R)-1-amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide (200 mg, 0.409 mmol, Intermediate 197), 1-isopropyl-1H-pyrazole-5-carboxylic acid (66 mg, 0.43 mmol), HOBt (68 mg, 0.43 mmol) and DIPEA (0.11 mL, 0.614 mmol) in ACN (1 mL) was heated to 40° C. for 5 min then EDCI (82 mg, 0.43 mmol) was added. The reaction was stirred at this temperature for 1 h then was poured into water. The mixture was extracted three times with EtOAc, then the combined organic layers were washed with water and brine, dried over anhydrous MgSO₄, filtered, and evaporated to a residue. The crude material was purified by silica gel chromatography (0-100% (10% MeOH/EtOAc)/hexanes) followed by preparative HPLC (XBridge, C18, OBD, 50×100 mm, 5-95% MeCN/H₂O with 20 mM NH₄OH) to provide the title compound. ¹H NMR (500 MHz, DMSO-d₆) δ 12.41-12.28 (m, 1H), 8.90-8.84 (m, 1H), 8.51-8.41 (m, 1H), 7.58-7.47 (m, 2H), 7.44-7.37 (m, 1H), 7.20-7.12 (m, 1H), 6.91 (d, J=2.0 Hz, 1H), 5.51-5.38 (m, 2H), 4.39-4.32 (m, 1H), 4.20-4.13 (m, 1H), 4.03-3.95 (m, 1H), 2.71-2.56 (m, 2H), 2.41-2.23 (m, 5H), 1.40-1.36 (m, 6H), 1.36-1.32 (m, 6H), 1.22-1.12 (m, 1H), 0.53-0.44 (m, 2H), 0.35-0.29 (m, 2H). MS (ESI) m/z: [M+H]⁺ Found 625.2.

### Example 161

### N—((R*)-1-(6-((R)-Cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-4-methyl-1,2,5-oxadiazole-3-carboxamide

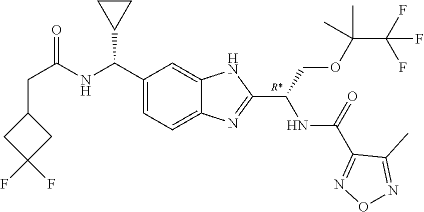 [SMILES: Cc1nonc1C(=O)N[C@@H](COC(C)(C)C(F)(F)F)c1nc2ccc([C@H](NC(=O)CC3CC(F)(F)C3)C3CC3)cc2[nH]1]

A solution of N—((R)-(2-((R)-1-amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide (50 mg, 0.102 mmol, Intermediate 197), 4-methyl-1,2,5-oxadiazole-3-carboxylic acid (14 mg, 0.113 mmol), HOBt (15 mg, 0.113 mmol) and DIPEA (0.03 mL, 0.164 mmol) in ACN (2 mL) was heated to 45° C. for 5 min then EDCI (22 mg, 0.113 mmol) was added. The reaction was stirred at this temperature for 2 h then was poured into water. The mixture was extracted three times with EtOAc. The combined organic layers were washed with saturated aqueous NH₄Cl, saturated aqueous NaHCO₃, and brine, dried over anhydrous Na₂SO₄, filtered and concentrated. The crude material was purified by silica gel chromatography (10-100% (10% MeOH/EtOAc)/hexanes) followed by preparative HPLC (XBridge C18, OBD, 50×100 mm, 5-95% ACN/H₂O with 0.5% NH₄OH) to provide the title compound. ¹H NMR (500 MHz, DMSO-d₆) δ 12.46-12.24 (m, 1H), 9.62-9.46 (m, 1H), 8.53-8.40 (m, 1H), 7.60-7.48 (m, 1H), 7.44-7.36 (m, 1H), 7.22-7.11 (m, 1H), 5.48-5.37 (m, 1H), 4.40-4.31 (m, 1H), 4.24-4.15 (m, 1H), 4.08-3.98 (m, 1H), 2.69-2.57 (m, 2H), 2.51 (s, 3H), 2.42-2.21 (m, 5H), 1.36-1.32 (m, 6H), 1.21-1.12 (m, 1H), 0.56-0.40 (m, 2H), 0.36-0.25 (m, 2H). MS (ESI) m/z: [M+H]⁺ Found 599.2.

### Example 162

### N—((S*)-1-(6-((R)-Cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-1-isopropyl-1H-pyrazole-5-carboxamide

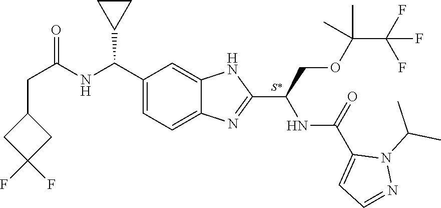 [SMILES: CC(C)n1nccc1C(=O)N[C@H](COC(C)(C)C(F)(F)F)c1nc2ccc([C@H](NC(=O)CC3CC(F)(F)C3)C3CC3)cc2[nH]1]

The title compound was prepared as described for the synthesis of Example 160, using N—((R)-(2-((S*)-1-amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide (Intermediate 203) in place of N—((R)-(2-((R)-1-amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide. ¹H NMR (500 MHz, DMSO-d₆) δ 12.34 (br s, 1H), 8.98-8.75 (m, 1H), 8.55-8.36 (m, 1H), 7.61-7.33 (m, 3H), 7.19-7.12 (m, 1H), 6.92-6.88 (m, 1H), 5.52-5.36 (m, 2H), 4.41-4.30 (m, 1H), 4.23-4.13 (m, 1H), 4.03-3.94 (m, 1H), 2.71-2.56 (m, 2H), 2.42-2.22 (m, 5H), 1.39-1.36 (m, 6H), 1.35-1.32 (m, 6H), 1.21-1.11 (m, 1H), 0.54-0.41 (m, 2H), 0.36-0.25 (m, 2H). MS (ESI) m/z: [M+H]⁺ Found 625.3.

### Example 163

### 4-Cyclopropyl-N—((S*)-1-(6-((R)-cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-1,2,5-oxadiazole-3-carboxamide

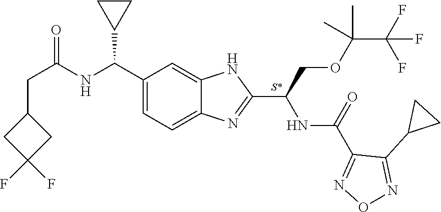 [SMILES: CC(C)(OC[C@@H](NC(=O)c1nonc1C1CC1)c1nc2ccc([C@H](NC(=O)CC3CC(F)(F)C3)C3CC3)cc2[nH]1)C(F)(F)F]

The title compound was prepared as described for the synthesis of Example 160, using N—((R)-(2-((S*)-1-amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide (Intermediate 203) in place of N—((R)-(2-((R)-1-amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide and 4-cyclopropyl-1,2,5-oxadiazole-3-carboxylic acid (Intermediate 78) in place of 1-isopropyl-1H-pyrazole-5-carboxylic acid. ¹H NMR (500 MHz, DMSO-d₆) δ 12.39 (s, 1H), 9.64-9.57 (m, 1H), 8.54-8.44 (m, 1H), 7.65-7.47 (m, 1H), 7.44-7.36 (m, 1H), 7.20-7.15 (m, 1H), 5.48-5.42 (m, 1H), 4.40-4.32 (m, 1H), 4.23-4.17 (m, 1H), 4.10-3.97 (m, 1H), 2.71-2.57 (m, 2H), 2.44-2.18 (m, 5H), 1.34 (s, 6H), 1.27-1.15 (m, 4H), 1.05-0.98 (m, 2H), 0.37-0.26 (m, 4H). MS (ESI) m/z: [M+H]⁺ Found 625.2.

### Example 164

### 4-Cyclopropyl-N—((R)-1-(5-((R*)-1-(2-(3,3-difluorocyclobutyl)acetamido)-2-methoxyethyl)-1H-benzo[d]imidazol-2-yl)-2-((1,1,1-trifluoro-2-methylpropan-2-yl)oxy)ethyl)-1,2,5-oxadiazole-3-carboxamide

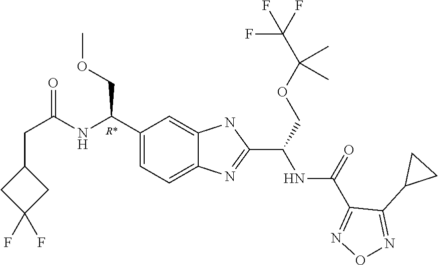 [SMILES: COC[C@H](NC(=O)CC1CC(F)(F)C1)c1ccc2nc([C@H](COC(C)(C)C(F)(F)F)NC(=O)c3nonc3C3CC3)[nH]c2c1]

### Example 165

### 4-Cyclopropyl-N—((R)-1-(5-((S*)-1-(2-(3,3-difluorocyclobutyl)acetamido)-2-methoxyethyl)-1H-benzo[d]imidazol-2-yl)-2-((1,1,1-trifluoro-2-methylpropan-2-yl)oxy)ethyl)-1,2,5-oxadiazole-3-carboxamide

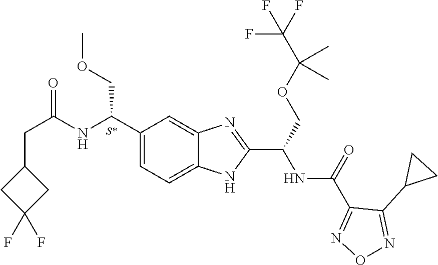 [SMILES: COC[C@@H](NC(=O)CC1CC(F)(F)C1)c1ccc2[nH]c([C@H](COC(C)(C)C(F)(F)F)NC(=O)c3nonc3C3CC3)nc2c1]

A solution of N-(1-(2-((R)-1-amino-2-((1,1,1-trifluoro-2-methylpropan-2-yl)oxy)ethyl)-1H-benzo[d]imidazol-5-yl)-2-methoxyethyl)-2-(3,3-difluorocyclobutyl)acetamide (123 mg, 0.25 mmol, Intermediate 411), 4-cyclopropyl-1,2,5-oxadiazole-3-carboxylic acid (77 mg, 0.5 mmol, Intermediate 78), HOBt (37 mg, 0.28 mmol) and DIPEA (0.09 mL, 0.5 mmol) in EtOAc (5 mL) was heated to 45° C. for 5 min then EDCI (53 mg, 0.28 mmol) was added. The reaction was stirred at this temperature for 1.5 h then diluted with EtOAc. The mixture was washed with saturated aqueous ammonium chloride, saturated aqueous sodium bicarbonate, and brine then dried over anhydrous Na₂SO₄, filtered and concentrated. The crude material was purified by silica gel chromatography (10-100% (10% MeOH/EtOAc)/hexanes). Further purification was done by SFC (Stationary phase: Whelk O1 SS, 5 μm, 250×21 mm, Mobile phase: 30% methanol with 0.2% triethylamine, 70% CO₂) to provide the title compounds. The first eluting isomer was Example 164 and the second eluting isomer was Example 165. Example 164: ¹H NMR (400 MHz, DMSO-d₆) δ 12.47-12.32 (m, 1H), 9.67-9.47 (m, 1H), 8.64-8.18 (m, 1H), 7.67-7.24 (m, 2H), 7.20-6.94 (m, 1H), 5.49-5.40 (m, 1H), 5.15-5.07 (m, 1H), 4.27-3.85 (m, 2H), 3.59-3.37 (m, 2H), 3.25 (s, 3H), 2.69-2.54 (m, 2H), 2.43-2.19 (m, 6H), 1.36-1.32 (m, 6H), 1.21-1.07 (m, 2H), 1.05-0.91 (m, 2H). MS (ESI) m/z: [M+H]⁺ Found 629.2. Example 165: ¹H NMR (400 MHz, DMSO-d₆) δ 12.52-12.31 (m, 1H), 9.81-9.43 (m, 1H), 8.52-8.28 (m, 1H), 7.75-7.29 (m, 2H), 7.20-7.04 (m, 1H), 5.56-5.36 (m, 1H), 5.19-5.00 (m, 1H), 4.27-4.10 (m, 1H), 4.10-3.97 (m, 1H), 3.62-3.42 (m, 2H), 3.26-3.18 (m, 3H), 2.70-2.54 (m, 2H), 2.44-2.23 (m, 6H), 1.39-1.29 (m, 6H), 1.21-0.96 (m, 4H). MS (ESI) m/z: [M+H]⁺ Found 629.2.

### Example 166

### 4-Cyclopropyl-N—((R)-1-(5-((R)-cyclopropyl(2-(3-fluorobicyclo[1.1.1]pentan-1-yl)acetamido)methyl)-1H-benzo[d]imidazol-2-yl)-2-((1,1,1-trifluoro-2-methylpropan-2-yl)oxy)ethyl)-1,2,5-oxadiazole-3-carboxamide

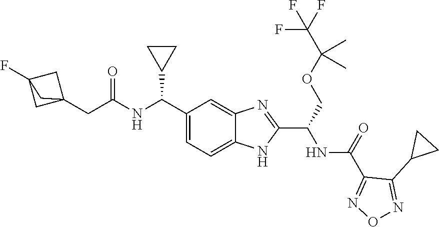 [SMILES: CC(C)(OC[C@H](NC(=O)c1nonc1C1CC1)c1nc2cc([C@H](NC(=O)CC34CC(F)(C3)C4)C3CC3)ccc2[nH]1)C(F)(F)F]

A solution of 4-cyclopropyl-1,2,5-oxadiazole-3-carboxylic acid (116 mg, 0.75 mmol, Intermediate 78), DIPEA (0.26 mL, 1.5 mmol) and EDCI (216 mg, 1.13 mmol) in CH₂Cl₂ (8 mL) was stirred for 10 min at rt then N—((R)-(2-((R)-1-amino-2-((1,1,1-trifluoro-2-methylpropan-2-yl)oxy)ethyl)-1H-benzo[d]imidazol-5-yl)(cyclopropyl)methyl)-2-(3-fluorobicyclo[1.1.1]pentan-1-yl)acetamide hydrochloride (195 mg, 0.38 mmol, Intermediate 205) was added. The reaction was stirred at rt overnight then diluted with H₂O (40 mL). The mixture was separated, and the aqueous phase was extracted with CH₂Cl₂ (3×30 mL). The combined organic phases were dried over anhydrous Na₂SO₄, filtered, and concentrated to dryness. Purification by silica gel chromatography (0-2% MeOH/DCM) then SFC (DAICEL CHIRALPAK AD (250×30 mm, 10 μm), (25% (v/v) 0.1% NH₄OH in EtOH) provided the title compound. ¹H NMR (400 MHz, DMSO-d₆) δ 12.40 (br s, 1H), 9.66-9.56 (m, 1H), 8.49-8.37 (m, 1H), 7.60-7.51 (m, 1H), 7.46-7.39 (m, 1H), 7.23-7.15 (m, 1H), 5.50-5.41 (m, 1H), 4.37-4.29 (m, 1H), 4.23-4.17 (m, 1H), 4.09-4.00 (m, 1H), 3.44-3.37 (m, 2H), 2.46-2.36 (m, 1H), 1.97-1.89 (m, 6H), 1.34 (s, 6H), 1.21-1.12 (m, 3H), 1.06-0.98 (m, 2H), 0.55-0.44 (m, 2H), 0.37-0.28 (m, 2H). MS (ESI) m/z: [M+H]⁺ Found 619.5.

### Example 167

### N—((R)-1-(5-((R)-Cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-1-(methyl-d₃)-1H-pyrazole-5-carboxamide

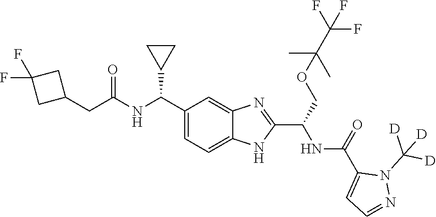 [SMILES: [2H]C([2H])([2H])n1nccc1C(=O)N[C@@H](COC(C)(C)C(F)(F)F)c1nc2cc([C@H](NC(=O)CC3CC(F)(F)C3)C3CC3)ccc2[nH]1]

To solution of N—((R)-(2-((R)-1-amino-2-((1,1,1-trifluoro-2-methylpropan-2-yl)oxy)ethyl)-1H-benzo[d]imidazol-6-yl)(cyclopropyl)methyl)-2-(3,3-difluorocyclobutyl)acetamide (118 mg, 0.242 mmol, Intermediate 197), 1-(methyl-d₃)-1H-pyrazole-5-carboxylic acid (62.5 mg, 0.484 mmol), HOBt (65.4 mg, 0.484 mmol) and DIPEA (125 mg, 0.968 mmol) in ACN (2 mL) was added EDCI (93 mg, 0.484 mmol). The reaction was stirred at rt overnight then diluted with CH₂Cl₂ and half saturated aqueous NH₄Cl. The layers were separated then the organic layer was washed with half-saturated aqueous NaHCO₃ solution and then brine solution, dried over anhydrous Na₂SO₄, filtered, and concentrated to dryness. Purification by silica gel chromatography (0-2% MeOH/DCM) then by SFC (DAICEL CHIRALCEL OD-H (250×30 mm, 5 μm), 20% (0.1% NH₄OH in EtOH)/80% supercritical CO₂) provided the title compound. ¹H NMR (400 MHz, DMSO-d₆) δ 12.36 (s, 1H), 8.95-8.91 (m, 1H), 8.52-8.47 (m, 1H), 7.58-7.50 (m, 1H), 7.49-7.48 (m, 1H), 7.44-7.35 (m, 1H), 7.18-7.15 (m, 1H), 6.98-6.97 (m, 1H), 5.43-5.37 (m, 1H), 4.38-4.32 (m, 1H), 4.20-4.17 (m, 1H), 4.01-3.97 (m, 1H), 2.62-2.58 (m, 2H), 2.42-2.19 (m, 5H), 1.34 (s, 6H), 1.26-1.12 (m, 1H), 0.49-0.47 (m, 2H), 0.31-0.29 (m, 2H). MS (ESI) m/z: [M+H]⁺ Found 600.2.

### Example 168

### N—((R)-1-(5-((R)-Cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-4-(2-hydroxypropan-2-yl)-1,2,5-oxadiazole-3-carboxamide

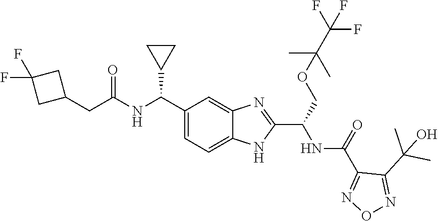 [SMILES: CC(C)(O)c1nonc1C(=O)N[C@@H](COC(C)(C)C(F)(F)F)c1nc2cc([C@H](NC(=O)CC3CC(F)(F)C3)C3CC3)ccc2[nH]1]

To a mixture of 6,6-dimethyl-4H,6H-furo[3,4-c][1,2,5]oxadiazol-4-one (36 mg, 0.233 mmol) and TBD (9.7 mg, 0.070 mmol) in THF (2 mL) was added N—((R)-(2-((R)-1-amino-2-((1,1,1-trifluoro-2-methylpropan-2-yl)oxy)ethyl)-1H-benzo[d]imidazol-6-yl)(cyclopropyl)methyl)-2-(3,3-difluorocyclobutyl)acetamide (114 mg, 0.233 mmol, Intermediate 197). The reaction was stirred for 16 h at 75° C. then diluted with H₂O (50 mL) and extracted with EtOAc (2×50 mL). The combined organics were washed with brine, dried over anhydrous Na₂SO₄, filtered and concentrated. Purification by silica gel chromatography (0-100% EtOAc/petroleum ether) followed by SFC (DAICEL CHIRALCEL PAK AD (250×30 mm, 10 μm; isocratic elution using 75% CO₂ in i-PrOH (0.1% NH₄OH))) provided the title compound. ¹H NMR (400 MHz, DMSO-d₆) δ 12.48 (s, 1H), 10.00 (s, 1H), 8.62-8.55 (m, 1H), 7.64-7.53 (m, 1H), 7.49 (s, 1H), 7.27-7.21 (m, 1H), 6.37 (s, 1H), 5.52 (s, 1H), 4.43-4.37 (m, 1H), 4.21-4.16 (m, 1H), 4.11-4.05 (m, 1H), 2.72-2.65 (m, 2H), 2.44-2.30 (m, 4H), 1.67 (s, 6H), 1.40 (s, 6H), 1.32-1.28 (m, 1H), 1.27-1.19 (m, 1H), 0.61-0.50 (m, 2H), 0.43-0.34 (m, 2H). MS (ESI) m/z: [M+H]⁺ Found 643.5.

### Example 169

### N—((S)-(6-((R)-Cyclopropyl(2-(3,3-difluorocyclobutyl)acetamido)methyl)-1H-benzo[d]imidazol-2-yl)(spiro[2.5]octan-6-yl)methyl)-4-methyl-1,2,5-oxadiazole-3-carboxamide

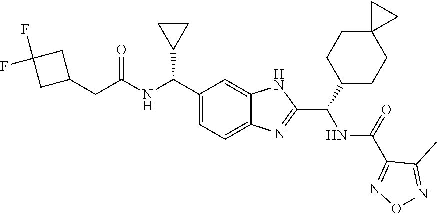 [SMILES: Cc1nonc1C(=O)N[C@H](c1nc2ccc([C@H](NC(=O)CC3CC(F)(F)C3)C3CC3)cc2[nH]1)C1CCC2(CC1)CC2]

PPTS (22 mg, 0.09 mmol) was added to a stirred solution of N—((S)-(5-((R)-cyclopropyl(2-(3,3-difluorocyclobutyl)acetamido)methyl)-1-((2-(trimethylsilyl)ethoxy)methyl)-1H-benzo[d]imidazol-2-yl)(spiro[2.5]octan-6-yl)methyl)-4-methyl-1,2,5-oxadiazole-3-carboxamide (40 mg, 0.06 mmol, Intermediate 213) in i-PrOH (2 mL), and the resulting mixture was stirred at 90° C. for 16 h. After this time, the mixture was diluted with EtOAc (20 mL) and washed with half-saturated aqueous sodium bicarbonate solution followed by brine. The organic layer was dried over anhydrous MgSO₄, filtered, and concentrated under reduced pressure to afford the crude product. Purification by SFC using a chiral stationary phase (DAICEL CHIRALPAK AS, 10 μm, 250×30 mm, 70% CO₂ in EtOH (0.1% NH₄OH)) afforded product containing fractions that were diluted with water, frozen, and lyophilized to afford the title compound as an off-white solid. ¹H NMR (400 MHz, DMSO-d₆) δ 12.31 (s, 1H), 9.47-9.43 (m, 1H), 8.49-8.44 (m, 1H), 7.59-7.48 (m, 1H), 7.44-7.38 (m, 1H), 7.21-7.13 (m, 1H), 5.13 (t, J=8.8 Hz, 1H), 4.35-4.30 (m, 1H), 2.70-2.57 (m, 2H), 2.47 (s, 3H), 2.38-2.28 (m, 4H), 2.19-2.06 (m, 1H), 1.87-1.84 (m, 1H), 1.72-1.53 (m, 2H), 1.39-1.36 (m, 1H), 1.30-1.11 (m, 4H), 0.99-0.82 (m, 2H), 0.54-0.42 (m, 2H), 0.35-0.13 (m, 6H). MS (ESI) m/z: [M+H]⁺ Found 567.2.

### Example 170

### 4-Cyclopropyl-N—((S)-(6-((R)-cyclopropyl(2-(3,3-difluorocyclobutyl)acetamido)methyl)-1H-benzo[d]imidazol-2-yl)((S)-3,3-difluorocyclohexyl)methyl)-1,2,5-oxadiazole-3-carboxamide

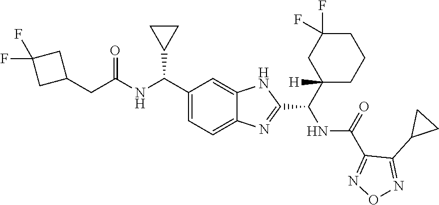 [SMILES: O=C(CC1CC(F)(F)C1)N[C@@H](c1ccc2nc([C@@H](NC(=O)c3nonc3C3CC3)[C@H]3CCCC(F)(F)C3)[nH]c2c1)C1CC1]

The title compound was prepared as described for the synthesis of Example 233, using N—((R)-(2-((S)-amino((R)-3,3-difluorocyclohexyl)methyl)-1H-benzo[d]imidazol-6-yl)(cyclopropyl)methyl)-2-(3,3-difluorocyclobutyl)acetamide (Intermediate 373) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 4-cyclopropyl-1,2,5-oxadiazole-3-carboxylic acid (Intermediate 78) in place of 4-methyl-1,2,5-oxadiazole-3-carboxylic acid. The product was purified by silica gel chromatography (0-2% MeOH/DCM) and further purified by SFC using a chiral stationary phase (DAICEL CHIRALCEL OD-H, 5 μm, 250×30 mm, 75% CO₂ in EtOH (0.1% NH₄OH)). The product containing fractions were diluted with water, frozen, and lyophilized to afford the title compound as a white solid. ¹H NMR (400 MHz, DMSO-d₆) δ 12.37 (s, 1H), 9.65-9.62 (m, 1H), 8.52-8.47 (m, 1H), 7.60-7.51 (m, 1H), 7.45-7.39 (m, 1H), 7.21-7.15 (m, 1H), 5.27-5.23 (m, 1H), 4.39-4.30 (m, 1H), 2.72-2.55 (m, 2H), 2.38-2.12 (m, 7H), 2.02-1.96 (m, 1H), 1.87-1.62 (m, 3H), 1.58-1.31 (m, 2H), 1.29-1.06 (m, 5H), 1.02-0.93 (m, 2H), 0.57-0.42 (m, 2H), 0.34-0.32 (m, 2H). MS (ESI) m/z: [M+H]⁺ Found 603.2.

### Example 171

### 4-Cyclopropyl-N—((S)-(6-((R)-cyclopropyl(2-(3,3-difluorocyclobutyl)acetamido)methyl)-1H-benzo[d]imidazol-2-yl)((R)-3,3-difluorocyclohexyl)methyl)-1,2,5-oxadiazole-3-carboxamide

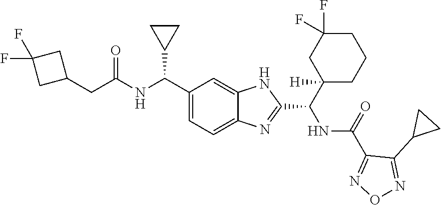 [SMILES: O=C(CC1CC(F)(F)C1)N[C@@H](c1ccc2nc([C@@H](NC(=O)c3nonc3C3CC3)[C@@H]3CCCC(F)(F)C3)[nH]c2c1)C1CC1]

The title compound was prepared as described for the synthesis of Example 233, using N—((R)-(2-((S)-amino((S)-3,3-difluorocyclohexyl)methyl)-1H-benzo[d]imidazol-6-yl)(cyclopropyl)methyl)-2-(3,3-difluorocyclobutyl)acetamide (Intermediate 375) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 4-cyclopropyl-1,2,5-oxadiazole-3-carboxylic acid (Intermediate 78) in place of 4-methyl-1,2,5-oxadiazole-3-carboxylic acid. The product was purified by preparative basic HPLC (Boston Prime, 5 μm, C18, 150×30 mm, 5-95% ACN/water (with 0.05% NH₄OH). The product containing fractions concentrated, diluted with water, frozen, and lyophilized to afford the title compound as white solid. ¹H NMR (400 MHz, DMSO-d₆) δ 12.40 (s, 1H), 9.65-9.56 (m, 1H), 8.56-8.47 (m, 1H), 7.64-7.50 (m, 1H), 7.48-7.39 (m, 1H), 7.18 (s, 1H), 5.28-5.18 (m, 1H), 4.37-4.29 (m, 1H), 2.69-2.59 (m, 2H), 2.46-2.23 (m, 8H), 2.01-1.94 (m, 1H), 1.89-1.61 (m, 4H), 1.49-1.35 (m, 1H), 1.24-1.17 (m, 2H), 1.15-1.10 (m, 2H), 1.00-0.95 (m, 2H), 0.54-0.44 (m, 2H), 0.38-0.28 (m, 2H). MS (ESI) m/z: [M+H]⁺ Found 603.2.

### Example 172

### N—((R)-(6-((R)-Cyclopropyl(2-(3,3-difluorocyclobutyl)acetamido)methyl)-1H-benzo[d]imidazol-2-yl)((S*)-4,4-difluorotetrahydro-2H-pyran-2-yl)methyl)-1-isopropyl-1H-pyrazole-5-carboxamide

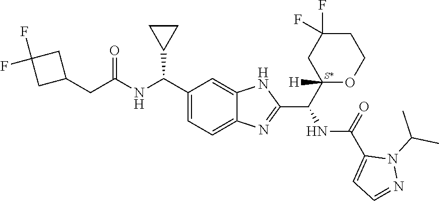 [SMILES: CC(C)n1nccc1C(=O)N[C@H](c1nc2ccc([C@H](NC(=O)CC3CC(F)(F)C3)C3CC3)cc2[nH]1)[C@@H]1CC(F)(F)CCO1]

The title compound was prepared as described for the synthesis of Example 233, using N—((R)-(2-((R)-amino((S*)-4,4-difluorotetrahydro-2H-pyran-2-yl)methyl)-1H-benzo[d]imidazol-6-yl)(cyclopropyl)methyl)-2-(3,3-difluorocyclobutyl)acetamide (Intermediate 216) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 1-isopropyl-1H-pyrazole-5-carboxylic acid in place of 4-methyl-1,2,5-oxadiazole-3-carboxylic acid. The crude product was purified by silica gel chromatography (0-100% EtOAc/petroleum ether) followed by preparative TLC (50% EtOAc/petroleum ether) to provide the title compound as a white solid. ¹H NMR (400 MHz, DMSO-d₆) δ 12.36 (s, 1H), 8.96-8.87 (m, 1H), 8.53-8.46 (m, 1H), 7.62-7.34 (m, 3H), 7.20-7.10 (m, 1H), 7.03-6.93 (m, 1H), 5.48-5.29 (m, 2H), 4.33 (t, J=8.4 Hz, 1H), 4.23-4.12 (m, 1H), 4.05-3.90 (m, 1H), 2.70-2.56 (m, 2H), 2.48-2.13 (m, 7H), 2.04-1.85 (m, 3H), 1.40-1.32 (m, 6H), 1.21-1.11 (m, 1H), 0.55-0.43 (m, 2H), 0.37-0.26 (m, 2H). MS (ESI) m/z: [M+H]⁺ Found 605.2.

### Example 173

### N—((S)-(6-((R)-Cyclopropyl(2-(3,3-difluorocyclobutyl)acetamido)methyl)-1H-benzo[d]imidazol-2-yl)((S*)-2,2-dimethyltetrahydro-2H-pyran-4-yl)methyl)-4-methyl-1,2,5-oxadiazole-3-carboxamide

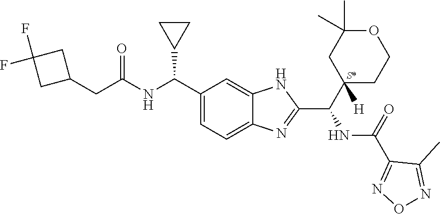 [SMILES: Cc1nonc1C(=O)N[C@H](c1nc2ccc([C@H](NC(=O)CC3CC(F)(F)C3)C3CC3)cc2[nH]1)[C@H]1CCOC(C)(C)C1]

### Example 174

### N—((S)-(6-((R)-Cyclopropyl(2-(3,3-difluorocyclobutyl)acetamido)methyl)-1H-benzo[d]imidazol-2-yl)((R*)-2,2-dimethyltetrahydro-2H-pyran-4-yl)methyl)-4-methyl-1,2,5-oxadiazole-3-carboxamide

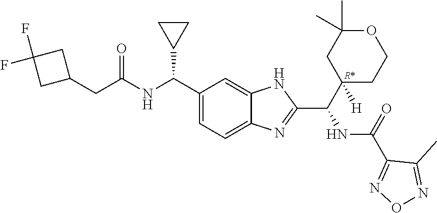 [SMILES: Cc1nonc1C(=O)N[C@H](c1nc2ccc([C@H](NC(=O)CC3CC(F)(F)C3)C3CC3)cc2[nH]1)[C@@H]1CCOC(C)(C)C1]

The title compounds were prepared as described for the synthesis of Example 233, using N-((1R)-(2-((1S)-amino(2,2-dimethyltetrahydro-2H-pyran-4-yl)methyl)-1H-benzo[d]imidazol-6-yl)(cyclopropyl)methyl)-2-(3,3-difluorocyclobutyl)acetamide (Intermediate 220) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude products were purified by preparative TLC (33% EtOAc/petroleum ether), and then the diastereomers were separated by SFC using a chiral stationary phase (DAICEL CHIRALPAK IG, 10 μm, 250×30 mm, 30% CO₂ in EtOH (0.1% NH₄OH)). The first eluting isomer was Example 173 and the second eluting isomer was Example 174. Example 173: ¹H NMR (400 MHz, DMSO-d₆) δ 12.35 (s, 1H), 9.57-9.45 (m, 1H), 8.58-8.43 (m, 1H), 7.59-7.50 (m, 1H), 7.46-7.39 (m, 1H), 7.22-7.10 (m, 1H), 5.07-4.97 (m, 1H), 4.38-4.27 (m, 1H), 3.70-3.61 (m, 1H), 3.59-3.53 (m, 1H), 2.68-2.57 (m, 2H), 2.47 (s, 3H), 2.40-2.20 (m, 5H), 1.79-1.68 (m, 1H), 1.28-1.12 (m, 5H), 1.12-1.05 (m, 6H), 0.55-0.40 (m, 2H), 0.37-0.27 (m, 2H). MS (ESI) m/z: [M+H]⁺ Found 571.5. Example 174: ¹H NMR (400 MHz, DMSO-d₆) δ 12.34 (s, 1H), 9.61-9.49 (m, 1H), 8.56-8.43 (m, 1H), 7.59-7.47 (m, 1H), 7.45-7.37 (m, 1H), 7.20-7.11 (m, 1H), 5.07-4.97 (m, 1H), 4.36-4.26 (m, 1H), 3.60-3.52 (m, 2H), 2.68-2.58 (m, 2H), 2.47 (s, 3H), 2.41-2.22 (m, 5H), 1.79-1.70 (m, 1H), 1.26-1.05 (m, 11H), 0.55-0.42 (m, 2H), 0.36-0.26 (m, 2H). MS (ESI) m/z: [M+H]⁺ Found 571.5.

### Example 175

### 4-Cyclopropyl-N—((R)-1-(5-((R)-cyclopropyl((R*)-4,4-difluoro-3-methylbutanamido)methyl)-1H-benzo[d]imidazol-2-yl)-2-((1,1,1-trifluoro-2-methylpropan-2-yl)oxy)ethyl)-1,2,5-oxadiazole-3-carboxamide

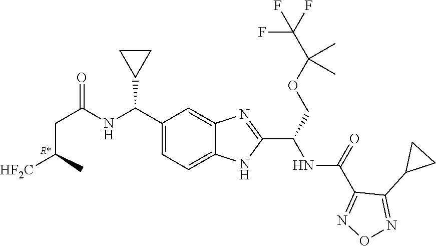

### Example 176

### 4-Cyclopropyl-N—((R)-1-(5-((R)-cyclopropyl((S*)-4,4-difluoro-3-methylbutanamido)methyl)-1H-benzo[d]imidazol-2-yl)-2-((1,1,1-trifluoro-2-methylpropan-2-yl)oxy)ethyl)-1,2,5-oxadiazole-3-carboxamide

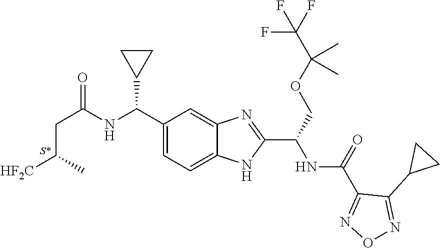

The title compounds were prepared as described for the synthesis of Example 159, using N—((R)-(2-((R)-1-amino-2-((1,1,1-trifluoro-2-methylpropan-2-yl)oxy)ethyl)-1H-benzo[d]imidazol-5-yl)(cyclopropyl)methyl)-4,4-difluoro-3-methylbutanamide (Intermediate 228) in place of N—((R)-(2-((R)-1-amino-2-((1,1,1-trifluoro-2-methylpropan-2-yl)oxy)ethyl)-1H-benzo[d]imidazol-5-yl)(cyclopropyl)methyl)-2-(3,3-difluorocyclobutyl)acetamide. The product was purified by silica gel chromatography (10-100% EtOAc (10% MeOH)/hexanes) to afford the product as a mixture of diastereomers, which were further separated by SFC using a chiral stationary phase (Whelk-01 (R,R), 10 μm, 250×20 mm, 85% CO₂ in EtOH (0.1% NH₄OH)). The first eluting isomer was Example 175 and the second eluting isomer was Example 176. Example 175: ¹H NMR (500 MHz, DMSO-d₆) δ 12.38 (s, 1H), 9.59 (d, J=8.3 Hz, 1H), 8.56 (dd, J=15.1, 8.4 Hz, 1H), 7.62-7.49 (m, 1H), 7.49-7.33 (m, 1H), 7.18 (t, J=8.0 Hz, 1H), 5.97 (td, J=56.9, 3.2 Hz, 1H), 5.53-5.36 (m, 1H), 4.38 (t, J=8.8 Hz, 1H), 4.19 (dd, J=9.7, 4.8 Hz, 1H), 4.03 (t, J=9.1 Hz, 1H), 2.43-2.33 (m, 3H), 2.09 (dd, J=14.1, 8.9 Hz, 1H), 1.34 (d, J=3.2 Hz, 6H), 1.20-1.13 (m, 3H), 1.03-0.98 (m, 2H), 0.85 (d, J=6.8 Hz, 3H), 0.54-0.43 (m, 2H), 0.37-0.26 (m, 2H). MS (ESI) m/z: [M+H]⁺ Found 613.3. Example 176: ¹H NMR (500 MHz, DMSO-d₆) δ 12.38 (s, 1H), 9.58 (t, J=7.1 Hz, 1H), 8.55 (dd, J=15.3, 8.5 Hz, 1H), 7.61-7.51 (m, 1H), 7.50-7.37 (m, 1H), 7.18 (t, J=8.1 Hz, 1H), 5.95 (td, J=57.0, 3.1 Hz, 1H), 5.45 (td, J=8.3, 4.8 Hz, 1H), 4.39 (t, J=8.5 Hz, 1H), 4.20 (dd, J=9.7, 4.8 Hz, 1H), 4.03 (t, J=9.1 Hz, 1H), 2.45-2.26 (m, 3H), 2.18-2.02 (m, 1H), 1.38-1.28 (m, 6H), 1.21-1.12 (m, 3H), 1.06-0.97 (m, 2H), 0.93 (d, J=6.7 Hz, 3H), 0.55-0.41 (m, 2H), 0.41-0.30 (m, 2H). MS (ESI) m/z: [M+H]⁺ Found 613.3.

### Example 177

### N—((S)-1-(6-((R)-Cyclopropyl(2-(3,3-difluorocyclobutyl)acetamido)methyl)-1H-benzo[d]imidazol-2-yl)-4,4,4-trifluoro-3,3-dimethylbutyl)-1-isopropyl-1H-pyrazole-5-carboxamide

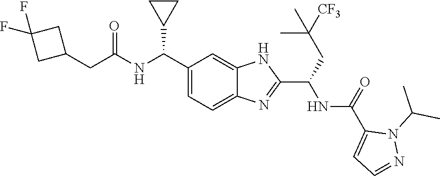 [SMILES: CC(C)n1nccc1C(=O)N[C@@H](CC(C)(C)C(F)(F)F)c1nc2ccc([C@H](NC(=O)CC3CC(F)(F)C3)C3CC3)cc2[nH]1]

A solution of N—((R)-(2-((S)-1-amino-4,4,4-trifluoro-3,3-dimethylbutyl)-1H-benzo[d]imidazol-6-yl)(cyclopropyl)methyl)-2-(3,3-difluorocyclobutyl)acetamide (65.0 mg, 0.128 mmol, Intermediate 255) in acetonitrile (1.5 mL) was added dropwise to a stirred solution of 1-isopropyl-1H-pyrazole-5-carboxylic acid (39.4 mg, 0.255 mmol), HOBt (20.7 mg, 0.153 mmol), EDCI (49.0 mg, 0.255 mmol) and DIPEA (44.0 μL, 0.255 mmol). The mixture was stirred at 25° C. for 16 h. The reaction mixture was concentrated under reduced pressure then diluted with water (10 mL) and extracted with DCM (20 mL×3). The combined organic layers were washed with brine (50 mL), dried over anhydrous sodium sulfate, filtered, and concentrated under reduced pressure. The crude material was purified by SFC (Daicel Chiralcel OD-H (250×30 mm, 5 μm) (isocratic elution: 20% EtOH (containing 0.1% NH₄OH) in supercritical CO₂)). The pure fractions were collected, concentrated under reduced pressure, frozen, and lyophilized to afford the title compound. ¹H NMR (400 MHz, DMSO-d₆) 12.33 (s, 1H), 9.11-8.97 (m, 1H), 8.59-8.46 (m, 1H), 7.60-7.49 (m, 2H), 7.45-7.36 (m, 1H), 7.21-7.11 (m, 1H), 6.96-6.83 (m, 1H), 5.54-5.42 (m, 2H), 4.38-4.29 (m, 1H), 2.71-2.54 (m, 3H), 2.42-2.20 (m, 6H), 1.38 (s, 6H), 1.19-1.13 (m, 7H), 0.55-0.43 (m, 2H), 0.35-0.26 (m, 2H). MS (ESI) m/z: [M+H]⁺ Found 609.3.

### Example 178

### 4-Cyclopropyl-N—((S)-1-(6-((R)-cyclopropyl(2-(3,3-difluorocyclobutyl)acetamido)methyl)-1H-benzo[d]imidazol-2-yl)-4,4,4-trifluoro-3,3-dimethylbutyl)-1,2,5-oxadiazole-3-carboxamide

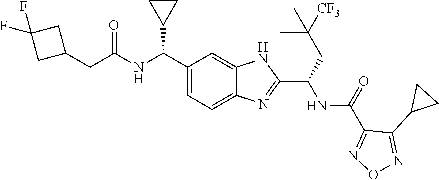 [SMILES: CC(C)(C[C@H](NC(=O)c1nonc1C1CC1)c1nc2ccc([C@H](NC(=O)CC3CC(F)(F)C3)C3CC3)cc2[nH]1)C(F)(F)F]

The title compound was prepared as described for the synthesis of Example 177, using 4-cyclopropyl-1,2,5-oxadiazole-3-carboxylic acid (Intermediate 78) in place of 1-isopropyl-1H-pyrazole-5-carboxylic acid. Purification by silica gel chromatography (0-50% EtOAc/petroleum ether) yielded the title compound. ¹H NMR (400 MHz, DMSO-d₆) δ 12.36 (s, 1H), 9.95-9.74 (m, 1H), 8.61-8.39 (m, 1H), 7.65-7.49 (m, 1H), 7.46-7.33 (m, 1H), 7.24-7.09 (m, 1H), 5.49 (s, 1H), 4.42-4.24 (m, 1H), 2.73-2.56 (m, 3H), 2.47-2.21 (m, 7H), 1.22-1.12 (m, 9H), 1.07-0.94 (m, 2H), 0.50 (s, 2H), 0.31 (s, 2H). MS (ESI) m/z: [M+H]⁺ Found 609.3.

### Example 179

### N—((S)-1-(6-((R)-Cyclopropyl((R*)-4,4,4-trifluoro-3-methylbutanamido)methyl)-1H-benzo[d]imidazol-2-yl)-4,4,4-trifluoro-3,3-dimethylbutyl)-4-methyl-1,2,5-oxadiazole-3-carboxamide

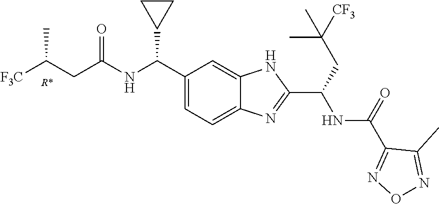 [SMILES: Cc1nonc1C(=O)N[C@@H](CC(C)(C)C(F)(F)F)c1nc2ccc([C@H](NC(=O)C[C@@H](C)C(F)(F)F)C3CC3)cc2[nH]1]

### Example 180

### N—((S)-1-(6-((R)-Cyclopropyl((S*)-4,4,4-trifluoro-3-methylbutanamido)methyl)-1H-benzo[d]imidazol-2-yl)-4,4,4-trifluoro-3,3-dimethylbutyl)-4-methyl-1,2,5-oxadiazole-3-carboxamide

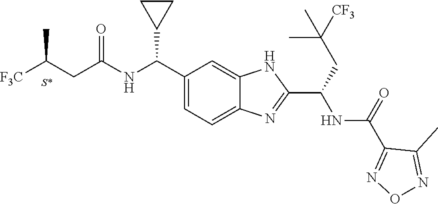 [SMILES: Cc1nonc1C(=O)N[C@@H](CC(C)(C)C(F)(F)F)c1nc2ccc([C@H](NC(=O)C[C@H](C)C(F)(F)F)C3CC3)cc2[nH]1]

The title compounds were prepared as described for the synthesis of Example 177, using N—((R)-(2-((S)-1-amino-4,4,4-trifluoro-3,3-dimethylbutyl)-1H-benzo[d]imidazol-6-yl)(cyclopropyl)methyl)-4,4,4-trifluoro-3-methylbutanamide (Intermediate 257) in place of N—((R)-(2-((S)-1-amino-4,4,4-trifluoro-3,3-dimethylbutyl)-1H-benzo[d]imidazol-6-yl)(cyclopropyl)methyl)-2-(3,3-difluorocyclobutyl)acetamide and 4-methyl-1,2,5-oxadiazole-3-carboxylic acid in place of 1-isopropyl-1H-pyrazole-5-carboxylic acid. Purification by SFC over Daicel Chiralpak IE (50×250 mm, 10 μm) (supercritical CO₂, 40% to 50% (v/v)) yielded the title compounds. The first eluting isomer was Example 179 and the second eluting isomer was Example 180. Example 179: ¹H NMR (400 MHz, DMSO-d₆) δ 12.31 (s, 1H), 9.83 (d, J=8.4 Hz, 1H), 8.72-8.61 (m, 1H), 7.61-7.50 (m, 1H), 7.45-7.36 (m, 1H), 7.22-7.14 (m, 1H), 5.52-5.40 (m, 1H), 4.38 (t, J=8.0 Hz, 1H), 2.83-2.69 (m, 1H), 2.63 (dd, J=3.6, 14.8 Hz, 1H), 2.53 (s, 3H), 2.48-2.41 (m, 1H), 2.35-2.20 (m, 2H), 1.25 (d, J=9.2 Hz, 1H), 1.18 (d, J=14.8 Hz, 6H), 1.07 (d, J=7.2 Hz, 3H), 0.50 (s, 2H), 0.33 (s, 2H). MS (ESI) m/z: [M+H]⁺ Found 589.1. Example 180: ¹H NMR (400 MHz, DMSO-d₆) δ 12.31 (s, 1H), 9.83 (d, J=8.4 Hz, 1H), 8.75-8.57 (m, 1H), 7.62-7.50 (m, 1H), 7.46-7.36 (m, 1H), 7.23-7.13 (m, 1H), 5.53-5.40 (m, 1H), 4.42-4.28 (m, 1H), 2.74 (s, 1H), 2.63 (dd, J=3.2, 14.8 Hz, 1H), 2.53 (s, 3H), 2.31 (dd, J=9.2, 14.4 Hz, 1H), 2.21 (ddd, J=4.4, 9.2, 14.4 Hz, 1H), 1.40-1.22 (m, 2H), 1.18 (d, J=14.4 Hz, 6H), 0.97 (d, J=6.8 Hz, 3H), 0.49 (t, J=8.8 Hz, 2H), 0.33 (s, 2H). MS (ESI) m/z: [M+H]⁺ Found 589.2

### Example 181

### 4-Cyclopropyl-N—((S)-1-(6-((R)-cyclopropyl((R*)-4,4,4-trifluoro-3-methylbutanamido)methyl)-1H-benzo[d]imidazol-2-yl)-4,4,4-trifluoro-3,3-dimethylbutyl)-1,2,5-oxadiazole-3-carboxamide

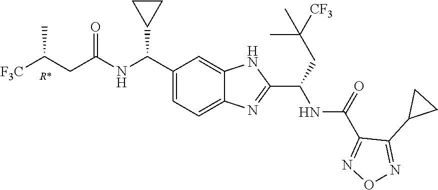 [SMILES: C[C@H](CC(=O)N[C@@H](c1ccc2nc([C@H](CC(C)(C)C(F)(F)F)NC(=O)c3nonc3C3CC3)[nH]c2c1)C1CC1)C(F)(F)F]

### Example 182

### 4-Cyclopropyl-N—((S)-1-(6-((R)-cyclopropyl((S*)-4,4,4-trifluoro-3-methylbutanamido)methyl)-1H-benzo[d]imidazol-2-yl)-4,4,4-trifluoro-3,3-dimethylbutyl)-1,2,5-oxadiazole-3-carboxamide

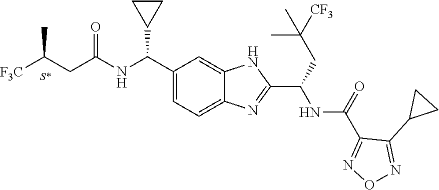 [SMILES: C[C@@H](CC(=O)N[C@@H](c1ccc2nc([C@H](CC(C)(C)C(F)(F)F)NC(=O)c3nonc3C3CC3)[nH]c2c1)C1CC1)C(F)(F)F]

The title compound was prepared as described for the synthesis of Example 177, using N—((R)-(2-((S)-1-amino-4,4,4-trifluoro-3,3-dimethylbutyl)-1H-benzo[d]imidazol-6-yl)(cyclopropyl)methyl)-4,4,4-trifluoro-3-methylbutanamide (Intermediate 257) in place of N—((R)-(2-((S)-1-amino-4,4,4-trifluoro-3,3-dimethylbutyl)-1H-benzo[d]imidazol-6-yl)(cyclopropyl)methyl)-2-(3,3-difluorocyclobutyl)acetamide and 4-cyclopropyl-1,2,5-oxadiazole-3-carboxylic acid (Intermediate 78) in place of 1-isopropyl-1H-pyrazole-5-carboxylic acid. Purification by SFC over DAICEL CHIRALPAK AS (250×30 mm, 10 μm), (isocratic elution: 15% EtOH (containing 0.1% NH₄OH) in supercritical CO₂) yielded the title compounds. The first eluting isomer was Example 182 and the second eluting isomer was Example 181. Example 181: ¹H NMR (400 MHz, DMSO-d₆) δ 12.36 (s, 1H), 9.90-9.78 (m, 1H), 8.75-8.60 (m, 1H), 7.61-7.50 (m, 1H), 7.45-7.37 (m, 1H), 7.22-7.16 (m, 1H), 5.55-5.42 (m, 1H), 5.23-4.30 (m, 1H), 2.81-2.68 (m, 1H), 2.66-2.58 (m, 1H), 2.50-2.39 (m, 2H), 2.36-2.16 (m, 2H), 1.22-1.14 (m, 9H), 1.09-0.99 (m, 2H), 0.97 (d, J=7.2 Hz, 3H), 0.56-0.43 (m, 2H), 0.33 (s, 2H). MS (ESI) m/z: [M+H]⁺ Found 615.2. Example 182: ¹H NMR (400 MHz, DMSO-d₆) δ 12.36 (s, 1H), 9.93-9.72 (m, 1H), 8.73-8.60 (m, 1H), 7.70-7.31 (m, 2H), 7.25-7.12 (m, 1H), 5.58-5.39 (m, 1H), 4.46-4.33 (m, 1H), 2.84-2.70 (m, 1H), 2.67-2.57 (m, 1H), 2.48-2.39 (m, 2H), 2.35-2.19 (m, 2H), 1.22-1.15 (m, 9H), 1.07 (d, J=6.8 Hz, 3H), 1.05-0.93 (m, 2H), 0.56-0.45 (m, 2H), 0.39-0.28 (m, 2H). MS (ESI) m/z: [M+H]⁺ Found 615.2.

### Example 183

### N—((S)-1-(6-((R)-Cyclopropyl(2-((R*)-2,2-difluorocyclopropyl) acetamido) methyl)-1H-benzo[d]imidazol-2-yl)-4,4,4-trifluoro-3,3-dimethylbutyl)-4-methyl-1,2,5-oxadiazole-3-carboxamide

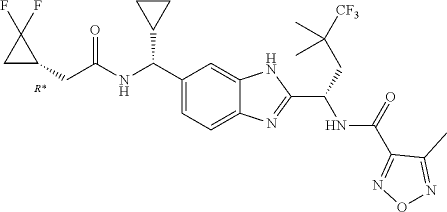 [SMILES: Cc1nonc1C(=O)N[C@@H](CC(C)(C)C(F)(F)F)c1nc2ccc([C@H](NC(=O)C[C@@H]3CC3(F)F)C3CC3)cc2[nH]1]

### Example 184

### N—((S)-1-(6-((R)-Cyclopropyl(2-((S*)-2,2-difluorocyclopropyl)acetamido) methyl)-1H-benzo[d]imidazol-2-yl)-4,4,4-trifluoro-3,3-dimethylbutyl)-4-methyl-1,2,5-oxadiazole-3-carboxamide

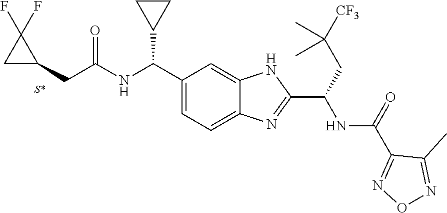 [SMILES: Cc1nonc1C(=O)N[C@@H](CC(C)(C)C(F)(F)F)c1nc2ccc([C@H](NC(=O)C[C@H]3CC3(F)F)C3CC3)cc2[nH]1]

The title compounds were prepared as described for the synthesis of Example 177, using N—((R)-(2-((S)-1-amino-4,4,4-trifluoro-3,3-dimethylbutyl)-1H-benzo[d]imidazol-6-yl)(cyclopropyl)methyl)-2-(2,2-difluorocyclopropyl)acetamide (Intermediate 259) in place of N—((R)-(2-((S)-1-amino-4,4,4-trifluoro-3,3-dimethylbutyl)-1H-benzo[d]imidazol-6-yl)(cyclopropyl)methyl)-2-(3,3-difluorocyclobutyl)acetamide and 4-methyl-1,2,5-oxadiazole-3-carboxylic in place of 1-isopropyl-1H-pyrazole-5-carboxylic acid. Purification by SFC over DAICEL CHIRALCEL OD-H (250×30 mm, 5 μm) (isocratic elution: 20% EtOH (0.1% NH₄OH IPA): 80% supercritical CO₂) yielded the title compounds. The first eluting isomer was Example 184 and the second eluting isomer was Example 183. Example 183: ¹H NMR (400 MHz, DMSO-d₆) δ 12.28 (s, 1H), 9.86-9.80 (m, 1H), 8.58-8.33 (m, 1H), 7.61-7.52 (m, 1H), 7.44-7.37 (m, 1H), 7.21-7.15 (m, 1H), 5.51-5.42 (m, 1H), 4.45-4.35 (m, 1H), 2.70-2.61 (m, 1H), 2.54 (s, 3H), 2.36-2.26 (m, 3H), 1.93-1.81 (m, 1H), 1.64-1.50 (m, 1H), 1.27-1.21 (m, 1H), 1.20 (s, 3H), 1.18-1.15 (m, 4H), 0.53-0.43 (m, 2H), 0.39-0.28 (m, 2H). MS (ESI) m/z: [M+H]⁺ Found 569.1. Example 184: ¹H NMR (400 MHz, DMSO-d₆) δ 12.28 (s, 1H), 9.82 (d, J=8.4 Hz, 1H), 8.52-8.46 (m, 1H), 7.63-7.50 (m, 1H), 7.46-7.36 (m, 1H), 7.22-7.18 (m, 1H), 5.51-5.43 (m, 1H), 4.44-4.36 (m, 1H), 2.69-2.60 (m, 1H), 2.54 (s, 3H), 2.43-2.24 (m, 3H), 1.92-1.76 (m, 1H), 1.60-1.45 (m, 1H), 1.20 (s, 3H), 1.18-1.15 (m, 4H), 1.15-1.10 (m, 1H), 0.54-0.43 (m, 2H), 0.38-0.28 (m, 2H). MS (ESI) m/z: [M+H]⁺ Found 569.2.

### Example 185

### 4-Cyclopropyl-N—((S)-1-(6-((R)-cyclopropyl(4,4-difluorobutanamido)methyl)-1H-benzo[d]imidazol-2-yl)-4,4,4-trifluoro-3,3-dimethylbutyl)-1,2,5-oxadiazole-3-carboxamide

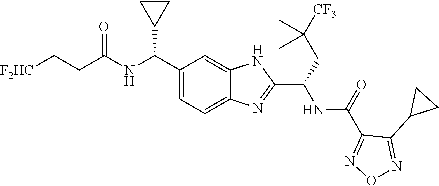 [SMILES: CC(C)(C[C@H](NC(=O)c1nonc1C1CC1)c1nc2ccc([C@H](NC(=O)CCC(F)F)C3CC3)cc2[nH]1)C(F)(F)F]

The title compound was prepared as described for the synthesis of Example 177, using N—((R)-(2-((S)-1-amino-4,4,4-trifluoro-3,3-dimethylbutyl)-1H-benzo[d]imidazol-6-yl)(cyclopropyl)methyl)-4,4-difluorobutanamide (Intermediate 261) in place of N—((R)-(2-((S)-1-amino-4,4,4-trifluoro-3,3-dimethylbutyl)-1H-benzo[d]imidazol-6-yl)(cyclopropyl)methyl)-2-(3,3-difluorocyclobutyl)acetamide and 4-cyclopropyl-1,2,5-oxadiazole-3-carboxylic acid (Intermediate 78) in place of 1-isopropyl-1H-pyrazole-5-carboxylic acid. Purification by silica gel chromatography (60-80% EtOAc/petroleum ether) yielded the title compound. ¹H NMR (400 MHz, DMSO-d₆) δ 12.33 (s, 1H), 9.85 (dd, J=4.0, 8.4 Hz, 1H), 8.53 (dd, J=8.4, 12.8 Hz, 1H), 7.60-7.51 (m, 1H), 7.44-7.37 (m, 1H), 7.20-7.14 (m, 1H), 6.23-5.89 (m, 1H), 5.52-5.43 (m, 1H), 4.40-4.31 (m, 1H), 2.69-2.60 (m, 1H), 2.47-2.39 (m, 1H), 2.35-2.22 (m, 3H), 2.08-1.96 (m, 2H), 1.24-1.15 (m, 9H), 1.07-0.95 (m, 2H), 0.54-0.42 (m, 2H), 0.36-0.26 (m, 2H). MS (ESI) m/z: [M+H]⁺ Found 583.2.

### Example 186

### N—((S)-1-(6-((R)-Cyclopropyl(2-(3,3-difluorocyclobutyl)acetamido)methyl)-1H-benzo[d]imidazol-2-yl)-4,4,4-trifluoro-3,3-dimethylbutyl)-4-methyl-1,2,5-oxadiazole-3-carboxamide

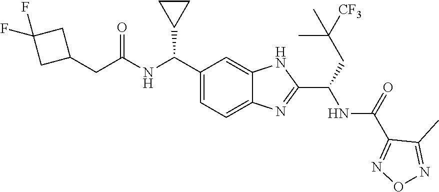 [SMILES: Cc1nonc1C(=O)N[C@@H](CC(C)(C)C(F)(F)F)c1nc2ccc([C@H](NC(=O)CC3CC(F)(F)C3)C3CC3)cc2[nH]1]

The title compound was prepared as described for the synthesis of Example 177, using 4-methyl-1,2,5-oxadiazole-3-carboxylic acid in place of 1-isopropyl-1H-pyrazole-5-carboxylic acid. Purification by silica gel chromatography (60-80% EtOAc/petroleum ether) then by SFC over DAICEL CHIRALPAK AD-H (250×30 mm, 5 μm) (20% EtOH (0.1% NH₄OH): 80% supercritical CO₂) yielded the title compound. ¹H NMR (400 MHz, DMSO-d₆) δ 12.28 (s, 1H), 9.82 (dd, J=5.2, 8.4 Hz, 1H), 8.47 (dd, J=8.4, 14.0 Hz, 1H), 7.61-7.51 (m, 1H), 7.43-7.37 (m, 1H), 7.20-7.13 (m, 1H), 5.51-5.42 (m, 1H), 4.41-4.30 (m, 1H), 2.71-2.56 (m, 3H), 2.54 (s, 3H), 2.43-2.26 (m, 6H), 1.28-1.21 (m, 1H), 1.20 (s, 3H), 1.16 (s, 3H), 0.54-0.43 (m, 2H), 0.37-0.23 (m, 2H). MS (ESI) m/z: [M+H]⁺ Found 583.3.

### Example 187

### N—((S)-(4,4-Difluorocyclohexyl)(5-((R)-1-(4,4,4-trifluorobutanamido)ethyl)-1H-benzo[d]imidazol-2-yl)methyl)-5-(3,3,3-trifluoropropyl)-1,3,4-oxadiazole-2-carboxamide

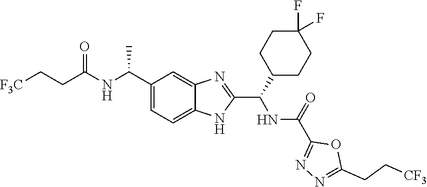 [SMILES: C[C@@H](NC(=O)CCC(F)(F)F)c1ccc2[nH]c([C@@H](NC(=O)c3nnc(CCC(F)(F)F)o3)C3CCC(F)(F)CC3)nc2c1]

N—((R)-1-(2-((S)-Amino(4,4-difluorocyclohexyl)methyl)-1H-benzo[d]imidazol-5-yl)ethyl)-4,4,4-trifluorobutanamide (160 mg, 0.370 mmol, Intermediate 4), lithium 5-(3,3,3-trifluoropropyl)-1,3,4-oxadiazole-2-carboxylate (181 mg, 0.840 mmol, Intermediate 271), HATU (281 mg, 0.740 mmol), DIPEA (190 μL, 1.11 mmol) and DMF (20 mL) were combined and stirred for 1 h under a nitrogen atmosphere. The reaction mixture was diluted with water (30 mL) and extracted with EtOAc (20 mL×3). The combined organic phases were washed brine (20 mL) dried over anhydrous sodium sulfate, filtered, and concentrated to dryness under reduced pressure. The crude material was purified via preparative HPLC using a Boston Prime C18 150×30 mm×5 μm column (45-75% (v/v) acetonitrile and water (0.04% NH₄OH and 10 mM NH₄HCO₃). The product was suspended in water (10 mL), the mixture frozen using dry ice/EtOH, and then lyophilized to dryness to afford the title compound. ¹H NMR (400 MHz, DMSO-d₆) δ 12.35 (s, 1H), 9.70 (d, J=8.0 Hz, 1H), 8.56-8.32 (m, 1H), 7.57-7.48 (m, 1H), 7.45-7.36 (m, 1H), 7.13 (t, J=8.8 Hz, 1H), 5.13 (t, J=8.4 Hz, 1H), 5.00 (d, J=7.6 Hz, 1H), 3.24 (t, J=7.6 Hz, 2H), 2.93-2.76 (m, 2H), 2.47-2.22 (m, 5H), 2.11-1.91 (m, 3H), 1.89-1.68 (m, 2H), 1.62-1.48 (m, 1H), 1.43-1.29 (m, 4H), 1.28-1.22 (m, 1H). MS (ESI) m/z: [M+H]⁺ Found 625.1.

### Example 188

### N—((S)-(4,4-Difluorocyclohexyl)(5-((R)-1-(4,4,4-trifluorobutanamido)ethyl)-1H-benzo[d]imidazol-2-yl)methyl)-4-isopropyl-1,2,3-thiadiazole-5-carboxamide

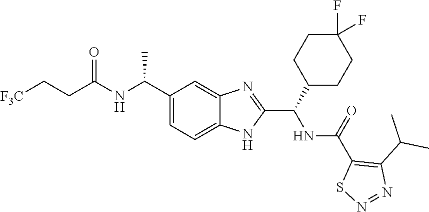 [SMILES: CC(C)c1nnsc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 177, using N—((R)-1-(2-((S)-amino(4,4-difluorocyclohexyl)methyl)-1H-benzo[d]imidazol-5-yl)ethyl)-4,4,4-trifluorobutanamide (Intermediate 4) in place of N—((R)-(2-((S)-1-amino-4,4,4-trifluoro-3,3-dimethylbutyl)-1H-benzo[d]imidazol-6-yl)(cyclopropyl)methyl)-2-(3,3-difluorocyclobutyl)acetamide and 4-isopropyl-1,2,3-thiadiazole-5-carboxylic acid in place of 1-isopropyl-1H-pyrazole-5-carboxylic acid. Purification by preparative HPLC with a Boston Green ODS 150×30 mm×5 μm column (37-67% (v/v) ACN in H₂O with 0.2% HCOOH) to afford pure product. The product was suspended in water (10 mL), the mixture frozen using dry ice/ethanol, and then lyophilized to dryness to afford the title compound. ¹H NMR (400 MHz, DMSO-d₆) δ 12.42 (s, 1H), 9.54 (d, J=8.40 Hz, 1H), 8.48-8.44 (m, 1H), 7.52-7.51 (m, 1H), 7.42-7.39 (m, 1H), 7.15-7.11 (m, 1H), 5.19 (t, J=8.0 Hz, 1H), 5.06-4.97 (m, 1H), 3.67-3.50 (m, 1H), 2.48-2.19 (m, 5H), 2.07-1.71 (m, 5H), 1.58 (d, J=15.2 Hz, 1H), 1.45-1.30 (m, 11H). MS (ESI) m/z: [M+H]⁺ Found 587.2.

### Example 189

### N—((S*)-1-(5-((S*)-Cyclopropyl(4,4,4-trifluorobutanamido)methyl)-1H-benzo[d]imidazol-2-yl)-4,4,4-trifluoro-3,3-dimethylbutyl)-4-ethyl-1,2,5-oxadiazole-3-carboxamide

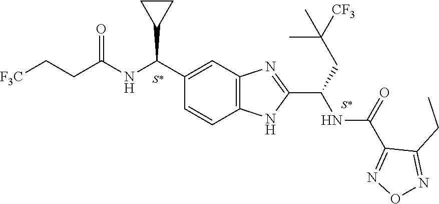 [SMILES: CCc1nonc1C(=O)N[C@@H](CC(C)(C)C(F)(F)F)c1nc2cc([C@@H](NC(=O)CCC(F)(F)F)C3CC3)ccc2[nH]1]

### Example 190

### N—((S*)-1-(5-((R*)-Cyclopropyl(4,4,4-trifluorobutanamido)methyl)-1H-benzo[d]imidazol-2-yl)-4,4,4-trifluoro-3,3-dimethylbutyl)-4-ethyl-1,2,5-oxadiazole-3-carboxamide

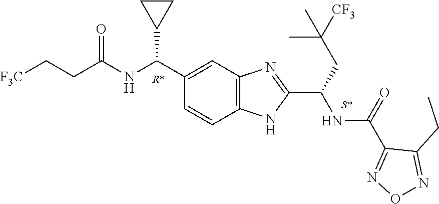 [SMILES: CCc1nonc1C(=O)N[C@@H](CC(C)(C)C(F)(F)F)c1nc2cc([C@H](NC(=O)CCC(F)(F)F)C3CC3)ccc2[nH]1]

N-((2-((S*)-1-Amino-4,4,4-trifluoro-3,3-dimethylbutyl)-1H-benzo[d]imidazol-5-yl)(cyclopropyl)methyl)-4,4,4-trifluorobutanamide (139 mg, 0.3 mmol, Intermediate 275), 4-ethyl-1,2,5-oxadiazole-3-carboxylic acid (47.5 mg, 0.33 mmol) and acetonitrile (5 mL) were combined followed by the addition of methyl imidazole (150 μL, 1.86 mmol) and chloro-N,N,N′,N′-tetramethylformamidinium hexafluorophosphate (106 mg, 0.380 mmol). The contents were stirred at rt overnight then transferred to a separatory funnel with EtOAc and deionized water. The organic phase was separated and the aqueous layer was extracted twice with EtOAc. The combined organic phases were dried over anhydrous magnesium sulfate, filtered, and concentrated to dryness under reduced pressure. Purification by silica gel chromatography (0-100% EtOAc/hexanes) then by SFC (AD-H (3×25 cm), 25% (1:1) heptane/ethanol in CO₂) yielded the title compounds. The first eluting isomer was Example 190 and the second eluting isomer was Example 189. Example 189: ¹H NMR (400 MHz, CD₃OD) δ 7.54-7.17 (m, 2H), 7.15-7.00 (m, 1H), 5.44 (dd, J=8.8, 4.5 Hz, 1H), 4.19 (d, J=8.5 Hz, 1H), 2.86-2.74 (m, 2H), 2.42 (dd, J=14.8, 4.5 Hz, 1H), 2.38-2.16 (m, 5H), 1.12 (t, J=7.5 Hz, 3H), 1.06 (s, 3H), 1.01 (s, 3H), 0.79-0.58 (m, 1H), 0.43-0.33 (m, 2H), 0.24-0.11 (m, 2H). MS (ESI) m/z: [M+H]⁺ Found 589.3. Example 190: ¹H NMR (400 MHz, CD₃OD) δ 7.51-7.18 (m, 2H), 7.07 (d, J=8.2 Hz, 1H), 5.44 (dd, J=8.8, 4.5 Hz, 1H), 4.19 (d, J=8.8 Hz, 1H), 2.86-2.74 (m, 2H), 2.42 (dd, J=14.8, 4.5 Hz, 1H), 2.38-2.16 (m, 5H), 1.14-1.10 (m, 3H), 1.07-1.05 (m, 3H), 1.02-1.00 (m, 3H), 0.78-0.60 (m, 1H), 0.43-0.34 (m, 2H), 0.27-0.11 (m, 2H). MS (ESI) m/z: [M+H]⁺ Found 589.3.

### Example 191

### 2-(Cyclopropylmethyl)-N—((R*)-4,4,4-trifluoro-3,3-dimethyl-1-(5-((R)-1-(4,4,4-trifluorobutanamido)ethyl)-1H-benzo[d]imidazol-2-yl)butyl)-2H-1,2,3-triazole-4-carboxamide

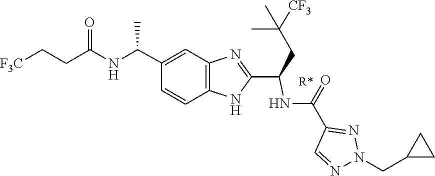 [SMILES: C[C@@H](NC(=O)CCC(F)(F)F)c1ccc2[nH]c([C@@H](CC(C)(C)C(F)(F)F)NC(=O)c3cnn(CC4CC4)n3)nc2c1]

The title compound was prepared as described for the synthesis of Example 189, using N—((R)-1-(2-((R*)-1-amino-4,4,4-trifluoro-3,3-dimethylbutyl)-1H-benzo[d]imidazol-6-yl)ethyl)-4,4,4-trifluorobutanamide (Intermediate 279) in place of N—((R)-(2-((S)-1-amino-4,4,4-trifluoro-3,3-dimethylbutyl)-1H-benzo[d]imidazol-6-yl)(cyclopropyl)methyl)-4,4-difluorobutanamide and 2-(cyclopropylmethyl)-2H-1,2,3-triazole-4-carboxylic acid (Intermediate 10) in place of 4-ethyl-1,2,5-oxadiazole-3-carboxylic acid. Purification by silica gel chromatography (0-100% EtOAc/hexanes) yielded the title compound. ¹H NMR (500 MHz, CD₃OD) δ 8.19 (s, 1H), 7.74-7.52 (m, 2H), 7.35 (dd, J=1.5, 8.5 Hz, 1H), 5.80 (dd, J=4.8, 8.8 Hz, 1H), 5.24 (q, J=6.8 Hz, 1H), 4.50-4.39 (m, 2H), 2.74-2.52 (m, 6H), 1.62 (d, J=7.0 Hz, 3H), 1.56-1.43 (m, 1H), 1.40-1.30 (m, 6H), 0.76-0.69 (m, 2H), 0.60-0.54 (m, 2H). MS (ESI) m/z: [M+H]⁺ Found 588.2.

### Example 192

### N—((S*)-1-(5-((S*)-Cyclopropyl(4,4,4-trifluorobutanamido)methyl)-1H-benzo[d]imidazol-2-yl)-4,4,4-trifluoro-3,3-dimethylbutyl)-4-methyl-1,2,5-oxadiazole-3-carboxamide

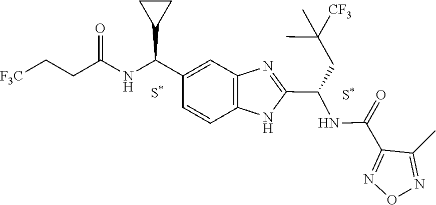 [SMILES: Cc1nonc1C(=O)N[C@@H](CC(C)(C)C(F)(F)F)c1nc2cc([C@@H](NC(=O)CCC(F)(F)F)C3CC3)ccc2[nH]1]

### Example 193

### N—((S*)-1-(5-((R*)-Cyclopropyl(4,4,4-trifluorobutanamido)methyl)-1H-benzo[d]imidazol-2-yl)-4,4,4-trifluoro-3,3-dimethylbutyl)-4-methyl-1,2,5-oxadiazole-3-carboxamide

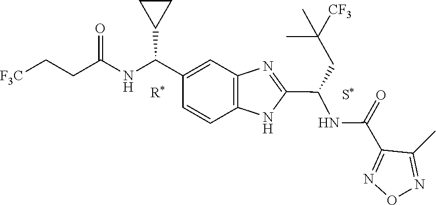 [SMILES: Cc1nonc1C(=O)N[C@@H](CC(C)(C)C(F)(F)F)c1nc2cc([C@H](NC(=O)CCC(F)(F)F)C3CC3)ccc2[nH]1]

The title compounds were prepared as described for the synthesis of Example 189, using 4-methyl-1,2,5-oxadiazole-3-carboxylic acid in place of 4-ethyl-1,2,5-oxadiazole-3-carboxylic acid. Purification by silica gel chromatography (0-100% EtOAc/hexanes) followed by purification via SFC (Stationary phase: Whelk O1 SS, 5 μm, 250×21 mm, Mobile phase: 25% methanol with 0.2% triethylamine, 75% CO₂) provided the title compounds. The first eluting isomer was Example 193 and the second eluting isomer was Example 192. Example 192: ¹H NMR (500 MHz, CD₃OD) δ 7.45 (br s, 1H), 7.42-7.31 (m, 1H), 7.15 (dd, J=1.50, 8.50 Hz, 1H), 5.59-5.46 (m, 1H), 4.28 (d, J=9.01 Hz, 1H), 2.55-2.49 (m, 1H), 2.47-2.25 (m, 8H), 1.17-1.07 (m, 7H), 0.52-0.43 (m, 2H), 0.33-0.21 (m, 2H). MS (ESI) m/z: [M+H]⁺ Found 575.2. Example 193: ¹H NMR (500 MHz, CD₃OD) δ 7.43 (br s, 1H), 7.10-7.31 (m, 1H), 7.14 (dd, J=1.50, 8.50 Hz, 1H), 5.56-5.45 (m, 1H), 4.26 (d, J=9.01 Hz, 1H), 2.52-2.47 (m, 1H), 2.44-2.22 (m, 8H), 1.17-1.03 (m, 7H), 0.50-0.40 (m, 2H), 0.31-0.19 (m, 2H). MS (ESI) m/z: [M+H]⁺ Found 575.2.

### Example 194

### 4-Fluoro-1-isopropyl-N—((S*)-4,4,4-trifluoro-3,3-dimethyl-1-(5-((R)-1-(4,4,4-trifluorobutanamido)ethyl)-1H-benzo[d]imidazol-2-yl)butyl)-1H-pyrazole-5-carboxamide

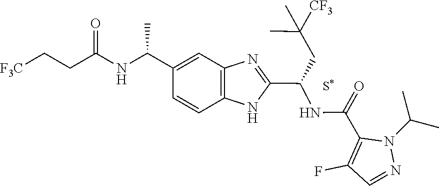 [SMILES: CC(C)n1ncc(F)c1C(=O)N[C@@H](CC(C)(C)C(F)(F)F)c1nc2cc([C@@H](C)NC(=O)CCC(F)(F)F)ccc2[nH]1]

The title compound was prepared as described for the synthesis of Example 187, using N—((R)-1-(2-((S*)-1-amino-4,4,4-trifluoro-3,3-dimethylbutyl)-1H-benzo[d]imidazol-5-yl)ethyl)-4,4,4-trifluorobutanamide (Intermediate 281) in place of N—((R)-1-(2-((S)-amino(4,4-difluorocyclohexyl)methyl)-1H-benzo[d]imidazol-5-yl)ethyl)-4,4,4-trifluorobutanamide and 4-fluoro-1-isopropyl-1H-pyrazole-5-carboxylic acid in place of lithium 5-(3,3,3-trifluoropropyl)-1,3,4-oxadiazole-2-carboxylate. Purification via preparative HPLC (C18, 50×250 mm, 5 μm; ACN/H₂O with 20 mM NH₄OH) provided the title compound. ¹H NMR (500 MHz, CD₃OD) δ 7.45-7.38 (m, 2H), 7.35 (d, J=4.5 Hz, 1H), 7.16-7.11 (m, 1H), 5.51 (dd, J=5.0, 8.3 Hz, 1H), 5.20-5.10 (m, 1H), 5.01 (q, J=6.9 Hz, 1H), 2.45-2.31 (m, 5H), 2.24 (dd, J=8.3, 14.8 Hz, 1H), 1.39 (d, J=7.0 Hz, 3H), 1.33-1.26 (m, 6H), 1.15 (s, 3H), 1.07 (s, 3H). MS (ESI) m/z: [M+H]⁺ Found 593.2.

### Example 195

### 1-(Cyclopropylmethyl)-N—((S*)-4,4,4-trifluoro-3,3-dimethyl-(5-((R)-1-(4,4,4-trifluorobutanamido)ethyl)-1H-benzo[d]imidazol-2-yl)butyl)-1H-1,2,4-triazole-5-carboxamide

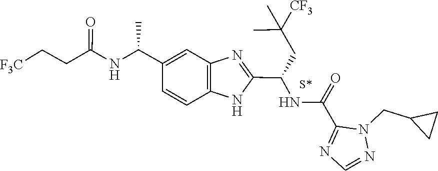 [SMILES: C[C@@H](NC(=O)CCC(F)(F)F)c1ccc2[nH]c([C@H](CC(C)(C)C(F)(F)F)NC(=O)c3ncnn3CC3CC3)nc2c1]

The title compound was prepared as described for the synthesis of Example 187, using N—((R)-1-(2-((S*)-1-amino-4,4,4-trifluoro-3,3-dimethylbutyl)-1H-benzo[d]imidazol-5-yl)ethyl)-4,4,4-trifluorobutanamide (Intermediate 281) in place of N—((R)-1-(2-((S)-amino(4,4-difluorocyclohexyl)methyl)-1H-benzo[d]imidazol-5-yl)ethyl)-4,4,4-trifluorobutanamide and 1-(cyclopropylmethyl)-1H-1,2,4-triazole-5-carboxylic acid (Intermediate 44) in place of lithium 5-(3,3,3-trifluoropropyl)-1,3,4-oxadiazole-2-carboxylate. The crude material was purified by preparative HPLC (Column: C18, 50×250 mm, 5 μm; ACN/H₂O with 20 mM NH₄OH followed by C18, 50×250 mm, 10 μm; ACN (with 0.05% TFA)/H₂O with 0.05% TFA). The pure material was filtered through a Silicycle® SiliaPrep™ Carbonate plug with methanol and concentrated to provide the title compound. ¹H NMR (500 MHz, CD₃OD) δ 7.85 (s, 1H), 7.41-7.37 (m, 2H), 7.14-7.10 (m, 1H), 5.49 (dd, J=4.8, 8.8 Hz, 1H), 5.00 (q, J=7.0 Hz, 1H), 4.47-4.38 (m, 1H), 4.38-4.29 (m, 1H), 2.48-2.43 (m, 1H), 2.42-2.31 (m, 4H), 2.30-2.23 (m, 1H), 1.38 (d, J=7.0 Hz, 3H), 1.29-1.17 (m, 1H), 1.13 (s, 3H), 1.08 (s, 3H), 0.41-0.28 (m, 4H). MS (ESI) m/z: [M+H]⁺ Found 588.2.

### Example 196

### 1-Isopropyl-N—((S*)-4,4,4-trifluoro-3,3-dimethyl-1-(5-((R)-1-(4,4,4-trifluorobutanamido)ethyl)-1H-benzo[d]imidazol-2-yl)butyl)-1H-1,2,4-triazole-5-carboxamide

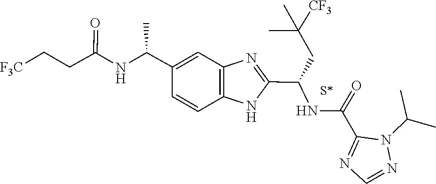 [SMILES: CC(C)n1ncnc1C(=O)N[C@@H](CC(C)(C)C(F)(F)F)c1nc2cc([C@@H](C)NC(=O)CCC(F)(F)F)ccc2[nH]1]

The title compound was prepared as described for the synthesis of Example 187, using N—((R)-1-(2-((S*)-1-amino-4,4,4-trifluoro-3,3-dimethylbutyl)-1H-benzo[d]imidazol-5-yl)ethyl)-4,4,4-trifluorobutanamide (Intermediate 281) in place of N—((R)-1-(2-((S)-amino(4,4-difluorocyclohexyl)methyl)-1H-benzo[d]imidazol-5-yl)ethyl)-4,4,4-trifluorobutanamide and 1-isopropyl-1H-1,2,4-triazole-5-carboxylic acid in place of lithium 5-(3,3,3-trifluoropropyl)-1,3,4-oxadiazole-2-carboxylate. The crude material was purified by preparative HPLC (Column: C18, 50×250 mm, 5 μm; Solvent: ACN/H₂O with 20 mM NH₄OH) to provide the title compound. ¹H NMR (500 MHz, DMSO-d₆) δ 12.32-12.14 (m, 1H), 9.50-9.35 (m, 1H), 8.51-8.34 (m, 1H), 8.14 (s, 1H), 7.60-7.32 (m, 2H), 7.15-7.10 (m, 1H), 5.67-5.55 (m, 1H), 5.52-5.40 (m, 1H), 5.07-4.97 (m, 1H), 2.65-2.52 (m, 1H), 2.49-2.35 (m, 5H), 1.46-1.41 (m, 6H), 1.40-1.37 (m, 3H), 1.18 (s, 3H), 1.14 (s, 3H). MS (ESI) m/z: [M+H]⁺ Found 576.2.

### Example 197

### 4-Ethyl-N—((S*)-4,4,4-trifluoro-3,3-dimethyl-1-(5-((R)-1-(4,4,4-trifluorobutanamido)ethyl)-1H-benzo[d]imidazol-2-yl)butyl)-1,2,5-oxadiazole-3-carboxamide

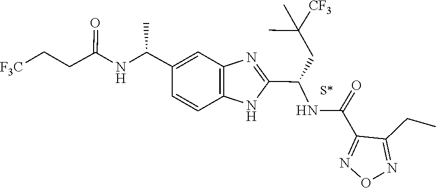 [SMILES: CCc1nonc1C(=O)N[C@@H](CC(C)(C)C(F)(F)F)c1nc2cc([C@@H](C)NC(=O)CCC(F)(F)F)ccc2[nH]1]

The title compound was prepared as described for the synthesis of Example 187, using N—((R)-1-(2-((S*)-1-amino-4,4,4-trifluoro-3,3-dimethylbutyl)-1H-benzo[d]imidazol-5-yl)ethyl)-4,4,4-trifluorobutanamide (Intermediate 281) in place of N—((R)-1-(2-((S)-amino(4,4-difluorocyclohexyl)methyl)-1H-benzo[d]imidazol-5-yl)ethyl)-4,4,4-trifluorobutanamide and 4-ethyl-1,2,5-oxadiazole-3-carboxylic acid in place of lithium 5-(3,3,3-trifluoropropyl)-1,3,4-oxadiazole-2-carboxylate. Purification by silica gel chromatography (0-100% EtOAc/hexanes) followed by preparative HPLC (Column: C18, 50×250 mm, 5 μm; Solvent: ACN/H₂O with 20 mM NH₄OH) provided the title compound. ¹H NMR (600 MHz, DMSO-d₆) δ 12.29 (s, 1H), 9.87-9.79 (m, 1H), 8.51-8.40 (m, 1H), 7.57-7.49 (m, 1H), 7.44-7.33 (m, 1H), 7.19-7.11 (m, 1H), 5.53-5.44 (m, 1H), 5.08-4.99 (m, 1H), 3.02-2.92 (m, 2H), 2.66-2.57 (m, 1H), 2.50-2.35 (m, 4H), 2.34-2.29 (m, 1H), 1.39 (d, J=6.9 Hz, 3H), 1.31-1.26 (m, 3H), 1.20 (s, 3H), 1.15 (s, 3H). MS (ESI) m/z: [M+H]⁺ Found 563.2.

### Example 198

### 4-Methyl-N—((S*)-4,4,4-trifluoro-3,3-dimethyl-1-(5-((R)-1-(4,4,4-trifluorobutanamido)ethyl)-1H-benzo[d]imidazol-2-yl)butyl)-1,2,5-oxadiazole-3-carboxamide

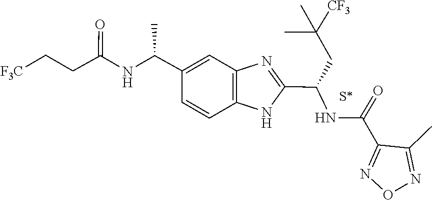 [SMILES: Cc1nonc1C(=O)N[C@@H](CC(C)(C)C(F)(F)F)c1nc2cc([C@@H](C)NC(=O)CCC(F)(F)F)ccc2[nH]1]

The title compound was prepared as described for the synthesis of Example 187, using N—((R)-1-(2-((S*)-1-amino-4,4,4-trifluoro-3,3-dimethylbutyl)-1H-benzo[d]imidazol-5-yl)ethyl)-4,4,4-trifluorobutanamide (Intermediate 281) in place of N—((R)-1-(2-((S)-amino(4,4-difluorocyclohexyl)methyl)-1H-benzo[d]imidazol-5-yl)ethyl)-4,4,4-trifluorobutanamide and 4-methyl-1,2,5-oxadiazole-3-carboxylic acid in place of lithium 5-(3,3,3-trifluoropropyl)-1,3,4-oxadiazole-2-carboxylate. Purification by silica gel chromatography (0-100% EtOAC/hexanes) followed by preparative HPLC (Column: C18, 50×250 mm, 5 μm; Solvent: ACN/H₂O with 20 mM NH₄OH) provided the title compound. ¹H NMR (600 MHz, DMSO-d₆) δ 12.31-12.23 (m, 1H), 9.83-9.78 (m, 1H), 8.50-8.40 (m, 1H), 7.56-7.50 (m, 1H), 7.42-7.34 (m, 1H), 7.17-7.11 (m, 1H), 5.50-5.44 (m, 1H), 5.06-4.99 (m, 1H), 2.67-2.61 (m, 1H), 2.54 (s, 3H), 2.50-2.35 (m, 4H), 2.34-2.29 (m, 1H), 1.39 (d, J=6.9 Hz, 3H), 1.20 (s, 3H), 1.16 (s, 3H). MS (ESI) m/z: [M+H]⁺ Found 549.1.

### Example 199

### N—((S)-(4,4-Difluorocyclohexyl)(5-((R)-1-(4,4,4-trifluorobutanamido)ethyl)-1H-benzo[d]imidazol-2-yl)methyl)-1-isopropyl-1H-1,2,4-triazole-5-carboxamide

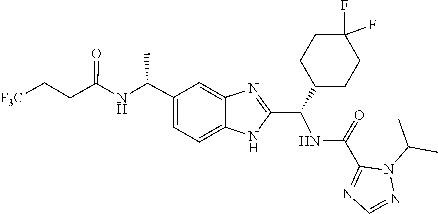 [SMILES: CC(C)n1ncnc1C(=O)N[C@H](c1nc2cc([C@@H](C)NC(=O)CCC(F)(F)F)ccc2[nH]1)C1CCC(F)(F)CC1]

To a mixture of N—((R)-1-(2-((S)-amino(4,4-difluorocyclohexyl)methyl)-1H-benzo[d]imidazol-5-yl)ethyl)-4,4,4-trifluorobutanamide (160 mg, 0.370 mmol, Intermediate 4) and methyl 1-isopropyl-1H-1,2,4-triazole-5-carboxylate (93 mg, 0.55 mmol, Intermediate 305) in toluene (5 mL) was added trimethylaluminum (0.74 mL, 1.5 mmol, 2 M in toluene) and the mixture was stirred at 90° C. for 2 h. The mixture was then cooled to rt then diluted with water (50 mL) and extracted with EtOAc (50 mL×2). The combined organic layers were dried over anhydrous sodium sulfate, filtered, and concentrated under reduced pressure. Purification by preparative HPLC (Column: Phenomenex Gemini-NX 150×30 mm×5 μm, Mobile Phase: 40-60% ACN/water (0.04% NH₄OH with 10 mM NH₄HCO₃)) then concentration under reduced pressure provided the title compound. ¹H NMR (400 MHz, DMSO-d₆) δ 12.38 (br s, 1H), 9.02 (d, J=8.6 Hz, 1H), 8.50-8.44 (m, 1H), 8.13 (s, 1H), 7.55-7.51 (m, 1H), 7.45-7.38 (m, 1H), 7.17-7.11 (m, 1H), 5.60-5.50 (m, 1H), 5.19-5.12 (m, 1H), 5.06-4.96 (m, 1H), 2.47-2.35 (m, 4H), 2.30-2.17 (m, 1H), 2.10-1.96 (m, 2H), 1.95-1.87 (m, 1H), 1.86-1.71 (m, 2H), 1.58-1.49 (m, 1H), 1.44-1.34 (m, 11H). MS (ESI) m/z: [M+H]⁺ Found 570.3.

### Example 200

### N—((S)-(4,4-Difluorocyclohexyl)(5-((R)-1-(4,4,4-trifluorobutanamido)ethyl)-1H-benzo[d]imidazol-2-yl)methyl)-5-isopropylthiazole-4-carboxamide

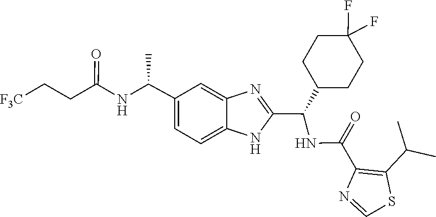 [SMILES: CC(C)c1scnc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 187, using 5-isopropylthiazole-4-carboxylic acid in place of lithium 5-(3,3,3-trifluoropropyl)-1,3,4-oxadiazole-2-carboxylate. Purification by preparative HPLC (Phenomenex Gemini-NX C18 75×35 mm×3 um column, (42-72% (v/v) ACN/water (0.04% NH₄OH with 10 mM NH₄HCO₃)) followed by lyophilization provided the title compound. ¹H NMR (400 MHz, DMSO-d₆) δ 12.43 (br s, 1H), 9.02 (s, 1H), 8.64-8.53 (m, 1H), 8.51-8.40 (m, 1H), 7.60-7.48 (m, 1H), 7.45-7.33 (m, 1H), 7.20-7.08 (m, 1H), 5.27-5.14 (m, 1H), 5.08-4.94 (m, 1H), 4.34-4.19 (m, 1H), 2.47-2.30 (m, 4H), 2.25-2.10 (m, 1H), 2.07-1.96 (m, 2H), 1.91-1.66 (m, 3H), 1.61-1.49 (m, 1H), 1.38 (d, J=7.1 Hz, 3H), 1.35-1.29 (m, 2H), 1.28-1.22 (m, 6H). MS (ESI) m/z: [M+H]⁺ Found 586.1.

### Example 201

### N—((S)-(4,4-Difluorocyclohexyl)(5-((R)-1-(4,4,4-trifluorobutanamido)ethyl)-1H-benzo[d]imidazol-2-yl)methyl)-4-(3,3,3-trifluoropropyl) thiazole-2-carboxamide

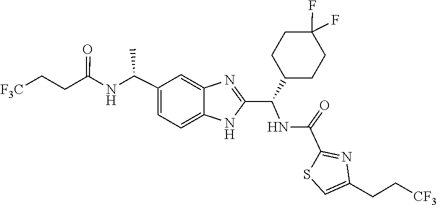 [SMILES: C[C@@H](NC(=O)CCC(F)(F)F)c1ccc2[nH]c([C@@H](NC(=O)c3nc(CCC(F)(F)F)cs3)C3CCC(F)(F)CC3)nc2c1]

The title compound was prepared as described for the synthesis of Example 188, using 4-(3,3,3-trifluoropropyl)thiazole-2-carboxylic acid (Intermediate 287) in place of 4-isopropyl-1,2,3-thiadiazole-5-carboxylic acid. Purification via preparative HPLC using a Phenomenex Gemini-NX, 150×30 mm×5 μm column (45-75% (v/v) ACN/water (0.04% NH₄OH with 10 mM NH₄HCO₃) followed by lyophilization yielded the title compound. ¹H NMR (400 MHz, DMSO-d₆) δ 12.39 (br s, 1H), 8.94 (d, J=9.0 Hz, 1H), 8.53-8.38 (m, 1H), 7.82 (s, 1H), 7.58-7.34 (m, 2H), 7.19-7.08 (m, 1H), 5.21-5.10 (m, 1H), 5.07-4.95 (m, 1H), 3.10-3.00 (m, 2H), 2.84-2.70 (m, 2H), 2.48-2.22 (m, 5H), 2.09-1.69 (m, 5H), 1.58-1.46 (m, 1H), 1.38 (d, J=7.0 Hz, 3H), 1.34-1.18 (m, 2H). MS (ESI) m/z: [M+H]⁺ Found 640.1.

### Example 202

### N—((S)-(4,4-Difluorocyclohexyl)(5-((R)-1-(4,4,4-trifluorobutanamido)ethyl)-1H-benzo[d]imidazol-2-yl)methyl)-5-isopropylisoxazole-4-carboxamide

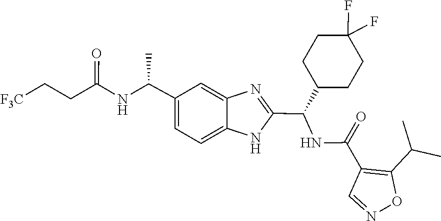 [SMILES: CC(C)c1oncc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 188, using 5-isopropylisoxazole-4-carboxylic acid in place of 4-isopropyl-1,2,3-thiadiazole-5-carboxylic acid. Purification via preparative HPLC using a Boston Prime C18, 150×30 mm×50 μm (50-80% (v/v) ACN/water (0.04% NH₄OH with 10 mM NH₄HCO₃)) followed by lyophilization yielded the title compound. ¹H NMR (400 MHz, DMSO-d₆) δ 12.37 (s, 1H), 9.08 (s, 1H), 8.84-8.76 (m, 1H), 8.55-8.39 (m, 1H), 7.55-7.30 (m, 2H), 7.17-7.05 (m, 1H), 5.21-5.12 (m, 1H), 5.07-4.93 (m, 1H), 3.88-3.74 (m, 1H), 2.47-2.30 (m, 4H), 2.30-2.17 (m, 1H), 2.09-1.92 (m, 3H), 1.88-1.71 (m, 2H), 1.59-1.49 (m, 1H), 1.46-1.40 (m, 1H), 1.38 (d, J=6.8 Hz, 3H), 1.35-1.27 (m, 1H), 1.26-1.20 (m, 6H). MS (ESI) m/z: [M+H]⁺ Found 570.1.

### Example 203

### N—((S)-(4,4-Difluorocyclohexyl)(5-((R)-1-(4,4,4-trifluorobutanamido)ethyl)-1H-benzo[d]imidazol-2-yl)methyl)-4-isopropylthiazole-5-carboxamide

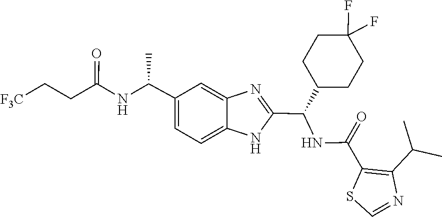 [SMILES: CC(C)c1ncsc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 188, using 4-isopropylthiazole-5-carboxylic acid in place of 4-isopropyl-1,2,3-thiadiazole-5-carboxylic acid. Purification via preparative HPLC using a Boston Prime C18, 150×30 mm×5 μm column (45-75% (v/v) CH₃CN/H₂O (with 0.04% NH₄OH plus 10 mM NH₄HCO₃)) followed by lyophilization yielded the title compound. ¹H NMR (400 MHz, DMSO-d₆) δ 12.32 (s, 1H), 9.05 (s, 1H), 8.85-8.74 (m, 1H), 8.50-8.39 (m, 1H), 7.54-7.48 (m, 1H), 7.44-7.36 (m, 1H), 7.17-7.08 (m, 1H), 5.16-5.10 (m, 1H), 5.07-4.95 (m, 1H), 3.71-3.58 (m, 1H), 2.49-2.34 (m, 4H), 2.31-2.18 (m, 1H), 2.11-1.89 (m, 3H), 1.88-1.67 (m, 2H), 1.57-1.48 (m, 1H), 1.43-1.35 (m, 4H), 1.30-1.23 (m, 1H), 1.22-1.17 (m, 6H). MS (ESI) m/z: [M+H]⁺ Found 586.2.

### Example 204

### N—((S)-(4,4-Difluorocyclohexyl)(5-((R)-1-(4,4,4-trifluorobutanamido)ethyl)-1H-benzo[d]imidazol-2-yl)methyl)-3-isopropylisoxazole-4-carboxamide

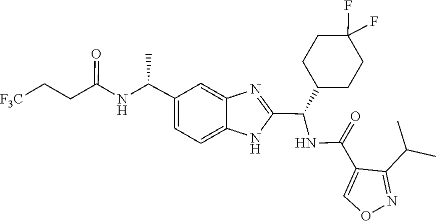 [SMILES: CC(C)c1nocc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 188, using 3-isopropylisoxazole-4-carboxylic acid in place of 4-isopropyl-1,2,3-thiadiazole-5-carboxylic acid. Purification via SFC (DAICEL CHIRALCEL OD column, 250×30 mm, 10 μm (isocratic elution: 20:80% (v/v) EtOH (containing 0.1% of 25% aqueous NH₃): supercritical CO₂) followed by lyophilization yielded the title compound. ¹H NMR (400 MHz, DMSO-d₆) δ 12.34 (s, 1H), 9.34 (s, 1H), 8.92-8.82 (m, 1H), 8.51-8.37 (m, 1H), 7.52-7.44 (m, 1H), 7.39-7.30 (m, 1H), 7.15-7.04 (m, 1H), 5.18-5.05 (m, 1H), 5.05-4.91 (m, 1H), 3.44-3.39 (m, 1H), 2.45-2.26 (m, 4H), 2.25-2.12 (m, 1H), 2.08-1.84 (m, 3H), 1.84-1.62 (m, 3H), 1.60-1.44 (m, 2H), 1.43-1.31 (m, 3H), 1.31-1.10 (m, 6H). MS (ESI) m/z: [M+H]⁺ Found 570.2.

### Example 205

### N—((S)-(4,4-Difluorocyclohexyl)(5-((R)-1-(4,4,4-trifluorobutanamido)ethyl)-1H-benzo[d]imidazol-2-yl)methyl)-3-isopropylthiophene-2-carboxamide

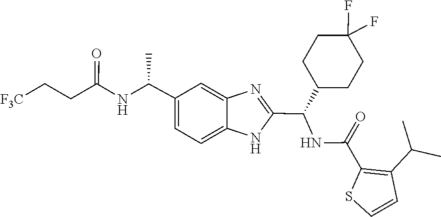 [SMILES: CC(C)c1ccsc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 188, using 3-isopropylthiophene-2-carboxylic acid in place of 4-isopropyl-1,2,3-thiadiazole-5-carboxylic acid. Purification via preparative HPLC with a Xtimate C18, 75×30 mm×3 μm column (45-75% (v/v) CH₃CN in H₂O (with 0.04% NH₄OH)) followed by lyophilization yielded the title compound. ¹H NMR (400 MHz, DMSO-d₆) δ 12.31 (br s, 1H), 8.54-8.38 (m, 2H), 7.62-7.55 (m, 1H), 7.54-7.49 (m, 1H), 7.44-7.37 (m, 1H), 7.18-7.10 (m, 2H), 5.18-5.10 (m, 1H), 5.05-4.98 (m, 1H), 3.67-3.55 (m, 1H), 2.48-2.35 (m, 4H), 2.29-2.17 (m, 1H), 2.12-1.89 (m, 3H), 1.88-1.64 (m, 2H), 1.56-1.46 (m, 1H), 1.43-1.34 (m, 4H), 1.32-1.23 (m, 1H), 1.19-1.10 (m, 6H). MS (ESI) m/z: [M+H]⁺ Found 585.3.

### Example 206

### N—((S)-(4,4-Difluorocyclohexyl)(5-((R)-1-(4,4,4-trifluorobutanamido)ethyl)-1H-benzo[d]imidazol-2-yl)methyl)-5-isopropyl-1,2,3-thiadiazole-4-carboxamide

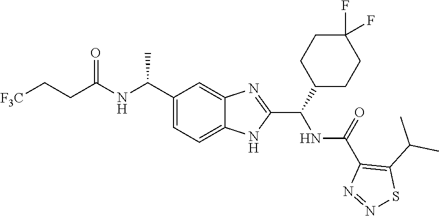 [SMILES: CC(C)c1snnc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 188, using 5-isopropyl-1,2,3-thiadiazole-4-carboxylic acid in place of 4-isopropyl-1,2,3-thiadiazole-5-carboxylic acid. Purification via preparative HPLC using a Xtimate C18, 150×30 mm×5 μm column (50-80% (v/v) ACN in H₂O (with 0.04% NH₄OH and 10 mM NH₄HCO₃)) followed by lyophilization yielded the title compound. ¹H NMR (400 MHz, DMSO-d₆) δ 12.38 (s, 1H), 9.21 (d, J=8.8 Hz, 1H), 8.52-8.39 (m, 1H), 7.57-7.49 (m, 1H), 7.45-7.35 (m, 1H), 7.19-7.06 (m, 1H), 5.32-5.20 (m, 1H), 5.07-4.95 (m, 1H), 4.15-4.04 (m, 1H), 2.47-2.35 (m, 4H), 2.31-2.21 (m, 1H), 2.10-1.90 (m, 3H), 1.89-1.68 (m, 2H), 1.62-1.40 (m, 2H), 1.38 (d, J=7.1 Hz, 3H), 1.34 (d, J=6.8 Hz, 3H), 1.30 (d, J=6.8 Hz, 3H), 1.27-1.11 (m, 1H). MS (ESI) m/z: [M+H]⁺ Found 587.2.

### Example 207

### N—((S)-(4,4-Difluorocyclohexyl)(5-((R)-1-(4,4,4-trifluorobutanamido)ethyl)-1H-benzo[d]imidazol-2-yl)methyl)-5-isopropyloxazole-4-carboxamide

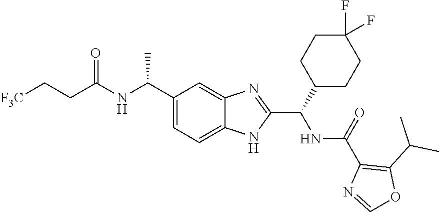 [SMILES: CC(C)c1ocnc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 188, using 5-isopropyloxazole-4-carboxylic acid in place of 4-isopropyl-1,2,3-thiadiazole-5-carboxylic acid. Purification via preparative HPLC using a Xtimate C18, 150×30 mm×5 μm column (50%-80% (v/v) ACN in H₂O (with 0.04% NH₄OH and 10 mM NH₄HCO₃)) followed by lyophilization provided the title compound. ¹H NMR (400 MHz, DMSO-d₆) δ 12.42-12.36 (m, 1H), 8.50-8.39 (m, 2H), 8.31-8.25 (m, 1H), 7.56-7.51 (m, 1H), 7.45-7.37 (m, 1H), 7.17-7.10 (m, 1H), 5.21-5.14 (m, 1H), 5.06-4.97 (m, 1H), 3.84-3.72 (m, 1H), 2.49-2.36 (m, 4H), 2.23-2.11 (m, 1H), 2.10-1.92 (m, 2H), 1.90-1.66 (m, 3H), 1.56-1.46 (m, 1H), 1.38 (d, J=6.8 Hz, 3H), 1.36-1.25 (m, 2H), 1.23 (d, J=7.0 Hz, 3H), 1.18 (d, J=7.0 Hz, 3H). MS (ESI) m/z: [M+H]⁺ Found 570.2.

### Example 208

### N—((S)-(4,4-Difluorocyclohexyl)(5-((R)-1-(4,4,4-trifluorobutanamido)ethyl)-1H-benzo[d]imidazol-2-yl)methyl)-1-isopropyl-1H-imidazole-5-carboxamide

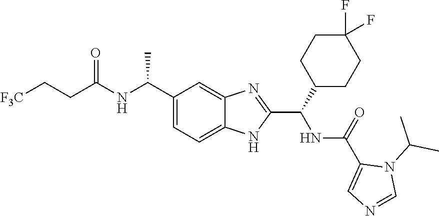 [SMILES: CC(C)n1cncc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 188, using 1-isopropyl-1H-imidazole-5-carboxylic acid in place of 4-isopropyl-1,2,3-thiadiazole-5-carboxylic acid. Purification via preparative HPLC using a Phenomenex Gemini-NX C18, 75×30 mm×3 μm column (26-56% (v/v) ACN in H₂O (with 0.04% NH₄OH and 10 mM NH₄HCO₃)) followed by lyophilization yielded the title compound. ¹H NMR (400 MHz, DMSO-d₆) δ 12.33 (br s, 1H), 8.70 (d, J=8.5 Hz, 1H), 8.53-8.45 (m, 1H), 7.99 (s, 1H), 7.73 (s, 1H), 7.53-7.48 (m, 1H), 7.42-7.36 (m, 1H), 7.16-7.09 (m, 1H), 5.22-5.09 (m, 2H), 5.06-4.96 (m, 1H), 2.49-2.32 (m, 4H), 2.30-2.19 (m, 1H), 2.10-1.92 (m, 3H), 1.89-1.67 (m, 2H), 1.58-1.49 (m, 1H), 1.42-1.35 (m, 10H), 1.30-1.20 (m, 1H). MS (ESI) m/z: [M+H]⁺ Found 569.2.

### Example 209

### 3-Cyclopropyl-N—((S)-(4,4-difluorocyclohexyl)(5-((R)-1-(4,4,4-trifluorobutanamido)ethyl)-1H-benzo[d]imidazol-2-yl)methyl)isoxazole-4-carboxamide

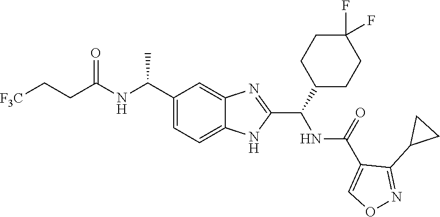 [SMILES: C[C@@H](NC(=O)CCC(F)(F)F)c1ccc2[nH]c([C@@H](NC(=O)c3conc3C3CC3)C3CCC(F)(F)CC3)nc2c1]

The title compound was prepared as described for the synthesis of Example 187, using 3-cyclopropylisoxazole-4-carboxylic acid in place of lithium 5-(3,3,3-trifluoropropyl)-1,3,4-oxadiazole-2-carboxylate. Purification by silica gel chromatography (0-100% DCM (10% (2 M NH₃ in MeOH) in DCM)) yielded the title compound. ¹H NMR (500 MHz, CD₃OD) δ 9.09 (s, 1H), 7.60-7.41 (m, 2H), 7.23 (dd, J=1.6, 8.4 Hz, 1H), 5.22 (d, J=8.3 Hz, 1H), 5.11 (q, J=7.0 Hz, 1H), 2.53-2.34 (m, 5H), 2.30-2.20 (m, 1H), 2.15-2.06 (m, 1H), 2.06-1.99 (m, 2H), 1.88-1.69 (m, 2H), 1.63-1.55 (m, 1H), 1.52 (d, J=3.0 Hz, 4H), 1.45-1.35 (m, 1H), 1.03-0.92 (m, 4H). MS (ESI) m/z: [M+H]⁺ Found 568.2.

### Example 210

### N—((S)-(4,4-Difluorocyclohexyl)(5-((R)-1-(4,4,4-trifluorobutanamido)ethyl)-1H-benzo[d]imidazol-2-yl)methyl)-2,3-dihydro-1H-pyrrolizine-7-carboxamide

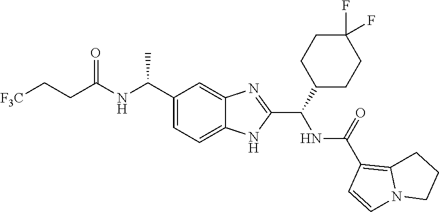 [SMILES: C[C@@H](NC(=O)CCC(F)(F)F)c1ccc2[nH]c([C@@H](NC(=O)c3ccn4c3CCC4)C3CCC(F)(F)CC3)nc2c1]

The title compound was prepared as described for the synthesis of Example 187, using 2,3-dihydro-1H-pyrrolizine-7-carboxylic acid in place of lithium 5-(3,3,3-trifluoropropyl)-1,3,4-oxadiazole-2-carboxylate. Purification by silica gel chromatography (0-100% DCM (10% (2 M NH₃ in MeOH) in DCM)) yielded the title compound. ¹H NMR (500 MHz, CD₃OD) δ 7.61-7.40 (m, 2H), 7.22 (dd, J=1.6, 8.4 Hz, 1H), 6.64-6.60 (m, 2H), 5.21 (d, J=8.5 Hz, 1H), 5.11 (q, J=7.0 Hz, 1H), 3.99-3.88 (m, 2H), 3.09-2.94 (m, 2H), 2.56-2.40 (m, 6H), 2.25-2.14 (m, 1H), 2.12-2.04 (m, 1H), 2.04-1.97 (m, 2H), 1.86-1.66 (m, 2H), 1.57-1.42 (m, 5H), 1.42-1.33 (m, 1H). MS (ESI) m/z: [M+H]⁺ Found 566.1.

### Example 211

### N—((S)-(4,4-Difluorocyclohexyl)(5-((R)-1-(4,4,4-trifluorobutanamido)ethyl)-1H-benzo[d]imidazol-2-yl)methyl)-6,7-dihydro-5H-pyrrolo[1,2-a]imidazole-3-carboxamide

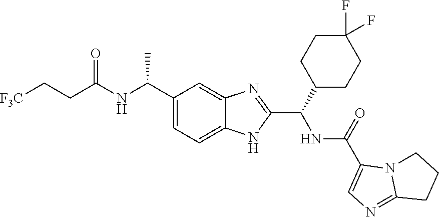 [SMILES: C[C@@H](NC(=O)CCC(F)(F)F)c1ccc2[nH]c([C@@H](NC(=O)c3cnc4n3CCC4)C3CCC(F)(F)CC3)nc2c1]

The title compound was prepared as described for the synthesis of Example 187, using 6,7-dihydro-5H-pyrrolo[1,2-a]imidazole-3-carboxylic acid hydrochloride in place of lithium 5-(3,3,3-trifluoropropyl)-1,3,4-oxadiazole-2-carboxylate. Purification by silica gel chromatography (0-100% DCM (10% (2 M NH₃ in MeOH) in DCM)) yielded the title compound. ¹H NMR (500 MHz, CD₃OD) δ 7.72 (s, 1H), 7.55-7.45 (m, 2H), 7.23 (dd, J=1.5, 8.5 Hz, 1H), 5.17 (d, J=8.8 Hz, 1H), 5.14-5.08 (m, 1H), 4.27-4.12 (m, 2H), 2.88-2.77 (m, 2H), 2.66-2.55 (m, 2H), 2.53-2.40 (m, 4H), 2.31-2.20 (m, 1H), 2.13-1.97 (m, 3H), 1.87-1.67 (m, 2H), 1.59-1.52 (m, 1H), 1.47 (d, J=3.5 Hz, 4H), 1.42-1.33 (m, 1H). MS (ESI) m/z: [M+H]⁺ Found 567.1.

### Example 212

### N—((S)-(4,4-Difluorocyclohexyl)(5-((R)-1-(4,4,4-trifluorobutanamido)ethyl)-1H-benzo[d]imidazol-2-yl)methyl)-5,6-dihydro-4H-pyrrolo[1,2-b]pyrazole-3-carboxamide

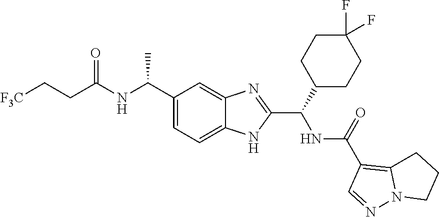 [SMILES: C[C@@H](NC(=O)CCC(F)(F)F)c1ccc2[nH]c([C@@H](NC(=O)c3cnn4c3CCC4)C3CCC(F)(F)CC3)nc2c1]

The title compound was prepared as described for the synthesis of Example 187, using 5,6-dihydro-4H-pyrrolo[1,2-b]pyrazole-3-carboxylic acid in place of lithium 5-(3,3,3-trifluoropropyl)-1,3,4-oxadiazole-2-carboxylate. Purification by silica gel chromatography (0-100% DCM (with 10% 2 M NH₃ in MeOH)/DCM) yielded the title compound. ¹H NMR (500 MHz, CD₃OD) δ 8.04 (s, 1H), 7.51 (br s, 2H), 7.23 (dd, J=1.8, 8.5 Hz, 1H), 5.23 (d, J=8.8 Hz, 1H), 5.12 (q, J=6.8 Hz, 1H), 4.15-4.05 (m, 2H), 3.13-2.99 (m, 2H), 2.70-2.56 (m, 2H), 2.55-2.41 (m, 4H), 2.31-2.20 (m, 1H), 2.14-1.98 (m, 3H), 1.87-1.67 (m, 2H), 1.63-1.53 (m, 1H), 1.53-1.46 (m, 4H), 1.45-1.35 (m, 1H). MS (ESI) m/z: [M+H]⁺ Found 567.2.

### Example 213

### N—((S)-(4,4-Difluorocyclohexyl)(5-((R)-1-(4,4,4-trifluorobutanamido)ethyl)-1H-benzo[d]imidazol-2-yl)methyl)-1-isopropyl-1H-pyrrole-2-carboxamide

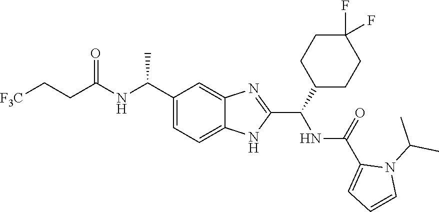 [SMILES: CC(C)n1cccc1C(=O)N[C@H](c1nc2cc([C@@H](C)NC(=O)CCC(F)(F)F)ccc2[nH]1)C1CCC(F)(F)CC1]

The title compound was prepared as described for the synthesis of Example 188, using 1-isopropyl-1H-pyrrole-2-carboxylic acid in place of 4-isopropyl-1,2,3-thiadiazole-5-carboxylic acid. Purification via preparative HPLC with a Xtimate C18, 150×40 mm×10 μm column (55-85% (v/v) ACN/water (0.04% NH₄OH with 10 mM NH₄HCO₃)) followed by lyophilization yielded the title compound. ¹H NMR (400 MHz, DMSO-d₆) δ 12.29 (br s, 1H), 8.53-8.40 (m, 1H), 8.36-8.24 (m, 1H), 7.55-7.46 (m, 1H), 7.44-7.33 (m, 1H), 7.20-7.06 (m, 2H), 6.95-6.86 (m, 1H), 6.12-6.03 (m, 1H), 5.44-5.29 (m, 1H), 5.20-5.07 (m, 1H), 5.06-4.94 (m, 1H), 2.48-2.31 (m, 4H), 2.29-2.13 (m, 1H), 2.08-1.90 (m, 3H), 1.88-1.68 (m, 2H), 1.57-1.49 (m, 1H), 1.48-1.40 (m, 1H), 1.38 (d, J=7.1 Hz, 3H), 1.33 (d, J=6.6 Hz, 3H), 1.28 (d, J=6.8 Hz, 3H), 1.26-1.17 (m, 1H). MS (ESI) m/z: [M+H]⁺ Found 568.3.

### Example 214

### N—((S)-(4,4-Difluorocyclohexyl)(5-((R)-1-(4,4,4-trifluorobutanamido)ethyl)-1H-benzo[d]imidazol-2-yl)methyl)-3-(3,3,3-trifluoropropyl)isoxazole-4-carboxamide

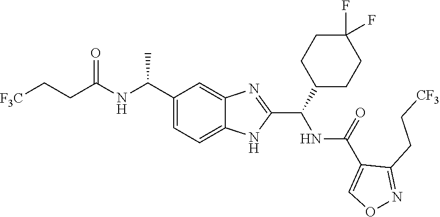 [SMILES: C[C@@H](NC(=O)CCC(F)(F)F)c1ccc2[nH]c([C@@H](NC(=O)c3conc3CCC(F)(F)F)C3CCC(F)(F)CC3)nc2c1]

### Example 215

### N—((S)-(4,4-Difluorocyclohexyl)(5-((R)-1-(4,4,4-trifluorobutanamido)ethyl)-1H-benzo[d]imidazol-2-yl)methyl)-3-(3,3,3-trifluoropropyl)isoxazole-5-carboxamide

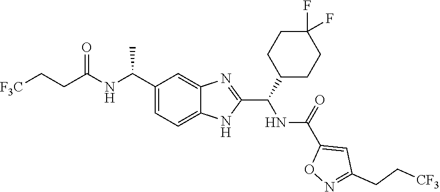 [SMILES: C[C@@H](NC(=O)CCC(F)(F)F)c1ccc2[nH]c([C@@H](NC(=O)c3cc(CCC(F)(F)F)no3)C3CCC(F)(F)CC3)nc2c1]

The title compounds were prepared as described for the synthesis of Example 188, using the mixture of 3-(3,3,3-trifluoropropyl)isoxazole-4-carboxylic acid and 3-(3,3,3-trifluoropropyl)isoxazole-5-carboxylic acid (Intermediate 290) in place of 4-isopropyl-1,2,3-thiadiazole-5-carboxylic acid. Purification via silica gel chromatography (9-100% EtOAc/petroleum ether) followed by SFC (DAICEL CHIRALPAK OD-H column (250×30 mm, 5 μm), solvent: 1:1 v/v EtOH (0.1% NH₄OH)/CO₂), yielded the title compounds. Example 214: ¹H NMR (400 MHz, DMSO-d₆): 12.49-12.37 (m, 1H), 9.57 (s, 1H), 9.10-8.98 (m, 1H), 8.52-8.40 (m, 1H), 7.54-7.48 (m, 1H), 7.42-7.36 (m, 1H), 7.17-7.09 (m, 1H), 5.22-5.14 (m, 1H), 5.06-4.97 (m, 1H), 3.13-3.05 (m, 2H), 2.75-2.62 (m, 2H), 2.48-2.32 (m, 4H), 2.29-2.18 (m, 1H), 2.09-1.90 (m, 3H), 1.89-1.70 (m, 2H), 1.63-1.52 (m, 1H), 1.46-1.28 (m, 5H). MS (ESI) m/z: [M+H]⁺ Found 624.2. Example 215: ¹H NMR (400 MHz, DMSO-d₆): 12.37 (br s, 1H), 9.42 (d, J=8.3 Hz, 1H), 8.53-8.41 (m, 1H), 7.57-7.47 (m, 1H), 7.44-7.36 (m, 1H), 7.23 (s, 1H), 7.18-7.09 (m, 1H), 5.19-5.09 (m, 1H), 5.06-4.96 (m, 1H), 3.02-2.92 (m, 2H), 2.81-2.66 (m, 2H), 2.46-2.24 (m, 5H), 2.12-1.91 (m, 3H), 1.88-1.69 (m, 2H), 1.59-1.50 (m, 1H), 1.42-1.25 (m, 5H). MS (ESI) m/z: [M+H]⁺ Found 624.2.

### Example 216

### N—((S)-(4,4-Difluorocyclohexyl)(5-((R)-1-(4,4,4-trifluorobutanamido)ethyl)-1H-benzo[d]imidazol-2-yl)methyl)-1-(3,3,3-trifluoropropyl)-1H-imidazole-5-carboxamide

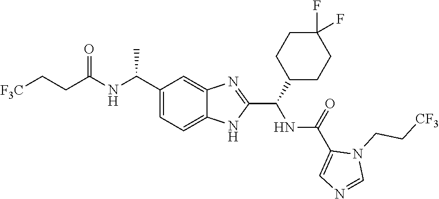 [SMILES: C[C@@H](NC(=O)CCC(F)(F)F)c1ccc2[nH]c([C@@H](NC(=O)c3cncn3CCC(F)(F)F)C3CCC(F)(F)CC3)nc2c1]

The title compound was prepared as described for the synthesis of Example 188, using 1-(3,3,3-trifluoropropyl)-1H-imidazole-2-carboxylic acid (Intermediate 292) in place of 4-isopropyl-1,2,3-thiadiazole-5-carboxylic acid. Purification via preparative HPLC with a Boston Prime C18, 150×30 mm×5 μm column (40-70% (v/v) CH₃CN in H₂O with 0.04% NH₄OH and 10 mM NH₄HCO₃) followed by lyophilization yielded the title compound. ¹H NMR (400 MHz, DMSO-d₆) δ 12.32 (s, 1H), 8.79 (d, J=8.3 Hz, 1H), 8.51-8.39 (m, 1H), 7.93-7.80 (m, 2H), 7.54-7.46 (m, 1H), 7.42-7.34 (m, 1H), 7.16-7.07 (m, 1H), 5.20-5.10 (m, 1H), 5.06-4.95 (m, 1H), 4.55-4.45 (m, 2H), 2.85-2.71 (m, 2H), 2.48-2.32 (m, 4H), 2.30-2.20 (m, 1H), 2.12-1.89 (m, 3H), 1.88-1.66 (m, 2H), 1.60-1.49 (m, 1H), 1.38 (d, J=7.1 Hz, 3H), 1.36-1.19 (m, 2H). MS (ESI) m/z: [M+H]⁺ Found 623.3.

### Example 217

### N—((S)-(4,4-Difluorocyclohexyl)(5-((R)-1-(4,4,4-trifluorobutanamido)ethyl)-1H-benzo[d]imidazol-2-yl)methyl)-5-(3,3,3-trifluoropropyl)isoxazole-3-carboxamide

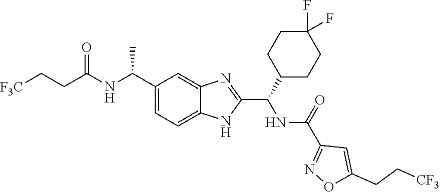 [SMILES: C[C@@H](NC(=O)CCC(F)(F)F)c1ccc2[nH]c([C@@H](NC(=O)c3cc(CCC(F)(F)F)on3)C3CCC(F)(F)CC3)nc2c1]

The title compound was prepared as described for the synthesis of Example 188, using 5-(3,3,3-trifluoropropyl)isoxazole-3-carboxylic acid in place of 4-isopropyl-1,2,3-thiadiazole-5-carboxylic acid. Purification by silica gel chromatography (0-50% EtOAc/petroleum ether) followed by SFC (DAICEL CHIRALPAK AD-H (250×30 mm×5 μm, 20/20, v/v MeOH (0.1% v/v NH₄OH)/CO₂) then lyophilization yielded the title compound. ¹H NMR (400 MHz, DMSO-d₆) δ 12.36 (br s, 1H), 9.09 (d, J=8.5 Hz, 1H), 8.58-8.39 (m, 1H), 7.58-7.48 (m, 1H), 7.46-7.36 (m, 1H), 7.20-7.08 (m, 1H), 6.78 (s, 1H), 5.20-5.12 (m, 1H), 5.07-4.95 (m, 1H), 3.11 (t, J=7.7 Hz, 2H), 2.85-2.69 (m, 2H), 2.49-2.32 (m, 4H), 2.32-2.20 (m, 1H), 2.11-1.90 (m, 3H), 1.88-1.67 (m, 2H), 1.56-1.47 (m, 1H), 1.39 (d, J=7.0 Hz, 3H), 1.37-1.18 (m, 2H). MS (ESI) m/z: [M+H]⁺ Found 624.3.

### Example 218

### 2-(Cyclopropylmethyl)-N—((S*)-4,4,4-trifluoro-3,3-dimethyl-1-(5-((R)-1-(4,4,4-trifluorobutanamido)ethyl)-1H-benzo[d]imidazol-2-yl)butyl)-2H-1,2,3-triazole-4-carboxamide

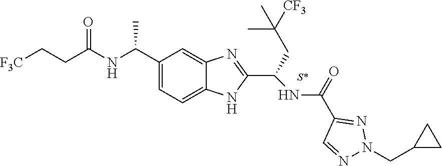 [SMILES: C[C@@H](NC(=O)CCC(F)(F)F)c1ccc2[nH]c([C@H](CC(C)(C)C(F)(F)F)NC(=O)c3cnn(CC4CC4)n3)nc2c1]

The title compound was prepared as described for the synthesis of Example 187, using N—((R)-1-(2-((S*)-1-amino-4,4,4-trifluoro-3,3-dimethylbutyl)-1H-benzo[d]imidazol-5-yl)ethyl)-4,4,4-trifluorobutanamide (Intermediate 281) in place of N—((R)-1-(2-((S)-amino(4,4-difluorocyclohexyl)methyl)-1H-benzo[d]imidazol-5-yl)ethyl)-4,4,4-trifluorobutanamide and 2-(cyclopropylmethyl)-2H-1,2,3-triazole-4-carboxylic acid (Intermediate 10) in place of lithium 5-(3,3,3-trifluoropropyl)-1,3,4-oxadiazole-2-carboxylate. Purification by silica gel chromatography (0-100% DCM (10% (2 M NH₃ in MeOH) in DCM)) yielded the title compound. ¹H NMR (500 MHz, CD₃OD) δ 8.21 (s, 1H), 7.64 (br s, 2H), 7.36 (dd, J=8.4, 1.6 Hz, 1H), 5.79 (dd, J=8.8, 4.8 Hz, 1H), 5.25 (q, J=6.8 Hz, 1H), 4.42-4.54 (m, 2H), 2.68-2.75 (m, 1H), 2.50-2.67 (m, 5H), 1.63 (d, J=7.0 Hz, 3H), 1.47-1.57 (m, 1H), 1.37 (s, 3H), 1.31-1.35 (m, 3H), 0.70-0.80 (m, 2H), 0.55-0.64 (m, 2H). MS (ESI) m/z: [M+H]⁺ Found 588.2.

### Example 219

### N—((S*)-4,4,4-Trifluoro-3,3-dimethyl-1-(5-((R)-1-(4,4,4-trifluorobutanamido)ethyl)-1H-benzo[d]imidazol-2-yl)butyl)-2-(3,3,3-trifluoropropyl)-2H-1,2,3-triazole-4-carboxamide

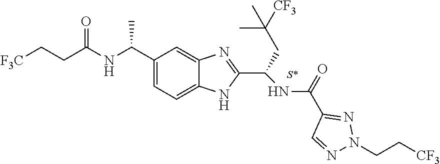 [SMILES: C[C@@H](NC(=O)CCC(F)(F)F)c1ccc2[nH]c([C@H](CC(C)(C)C(F)(F)F)NC(=O)c3cnn(CCC(F)(F)F)n3)nc2c1]

The title compound was prepared as described for the synthesis of Example 187, using N—((R)-1-(2-((S*)-1-amino-4,4,4-trifluoro-3,3-dimethylbutyl)-1H-benzo[d]imidazol-5-yl)ethyl)-4,4,4-trifluorobutanamide (Intermediate 281) in place of N—((R)-1-(2-((S)-amino(4,4-difluorocyclohexyl)methyl)-1H-benzo[d]imidazol-5-yl)ethyl)-4,4,4-trifluorobutanamide and 2-(3,3,3-trifluoropropyl)-2H-1,2,3-triazole-4-carboxylic acid (Intermediate 7) in place of lithium 5-(3,3,3-trifluoropropyl)-1,3,4-oxadiazole-2-carboxylate. Purification by silica gel chromatography (0-100% DCM (10% (2 M NH₃ in MeOH) in DCM)) yielded the title compound. ¹H NMR (500 MHz, CD₃OD) δ 8.11 (s, 1H), 7.45-7.58 (m, 2H), 7.24 (dd, J=1.5, 8.5 Hz, 1H), 5.62-5.72 (m, 1H), 5.08-5.15 (m, 1H), 4.74-4.80 (m, 2H), 2.91-3.03 (m, 2H), 2.55-2.65 (m, 1H), 2.38-2.54 (m, 5H), 1.50 (d, J=7.0 Hz, 3H), 1.24 (s, 3H), 1.20 (s, 3H). MS (ESI) m/z: [M+H]⁺ Found 630.2.

### Example 220

### N—((S)-(4,4-Difluorocyclohexyl)(5-((R)-1-(4,4,4-trifluorobutanamido)ethyl)-1H-benzo[d]imidazol-2-yl)methyl)-1-(3,3,3-trifluoropropyl)-1H-1,2,4-triazole-5-carboxamide

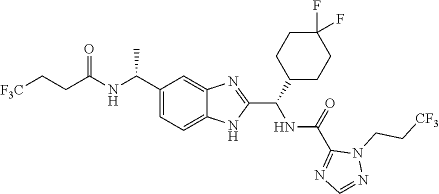 [SMILES: C[C@@H](NC(=O)CCC(F)(F)F)c1ccc2[nH]c([C@@H](NC(=O)c3ncnn3CCC(F)(F)F)C3CCC(F)(F)CC3)nc2c1]

The title compound was prepared as described for the synthesis of Example 199, using methyl 1-(3,3,3-trifluoropropyl)-1H-1,2,4-triazole-5-carboxylate (Intermediate 306) in place of methyl 1-isopropyl-1H-1,2,4-triazole-5-carboxylate. Purification via preparative HPLC with a YMC-Triart Prep C18, 250×50 mm×10 μm column (43-73% (v/v) CH₃CN in H₂O with 0.04% NH₄OH and 10 mM NH₄HCO₃) followed by lyophilization yielded the title compound. ¹H NMR (400 MHz, DMSO-d₆) δ 12.38 (s, 1H), 9.11 (d, J=8.8 Hz, 1H), 8.52-8.44 (m, 1H), 8.20 (s, 1H), 7.58-7.49 (m, 1H), 7.46-7.37 (m, 1H), 7.19-7.10 (m, 1H), 5.22-5.12 (m, 1H), 5.07-4.96 (m, 1H), 4.93-4.78 (m, 2H), 2.98-2.83 (m, 2H), 2.48-2.32 (m, 4H), 2.31-2.18 (m, 1H), 2.11-1.66 (m, 5H), 1.59-1.47 (m, 1H), 1.38 (d, J=6.8 Hz, 3H), 1.36-1.22 (m, 2H). MS (ESI) m/z: [M+H]⁺ Found 624.3.

### Example 221

### N—((S)-(4,4-Difluorocyclohexyl)(5-((R)-1-(4,4,4-trifluorobutanamido)ethyl)-1H-benzo[d]imidazol-2-yl)methyl)-5-(3,3,3-trifluoropropyl)thiophene-2-carboxamide

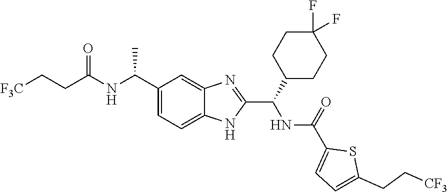 [SMILES: C[C@@H](NC(=O)CCC(F)(F)F)c1ccc2[nH]c([C@@H](NC(=O)c3ccc(CCC(F)(F)F)s3)C3CCC(F)(F)CC3)nc2c1]

The title compound was prepared as described for the synthesis of Example 188, using 5-(3,3,3-trifluoropropyl)thiophene-2-carboxylic acid (Intermediate 294) in place of 4-isopropyl-1,2,3-thiadiazole-5-carboxylic acid. Purification via preparative HPLC with a Boston Prime C18, 150×25 mm×5 μm column (50-80% (v/v) CH₃CN in H₂O with 0.04% NH₄OH and 10 mM NH₄HCO₃) afforded the title compound. ¹H NMR (400 MHz, DMSO-d₆) δ 12.44-12.30 (m, 1H), 8.89 (d, J=8.3 Hz, 1H), 8.52-8.40 (m, 1H), 7.83 (d, J=3.5 Hz, 1H), 7.54-7.47 (m, 1H), 7.43-7.34 (m, 1H), 7.16-7.08 (m, 1H), 7.02 (d, J=3.8 Hz, 1H), 5.16-5.08 (m, 1H), 5.06-4.95 (m, 1H), 3.11-3.00 (m, 2H), 2.74-2.61 (m, 2H), 2.47-2.22 (m, 5H), 2.12-1.92 (m, 3H), 1.89-1.67 (m, 2H), 1.59-1.48 (m, 1H), 1.44-1.24 (m, 5H). MS (ESI) m/z: [M+H]⁺ Found 639.1.

### Example 222

### N—((S)-(4,4-Difluorocyclohexyl)(5-((R)-1-(4,4,4-trifluorobutanamido)ethyl)-1H-benzo[d]imidazol-2-yl)methyl)-2-(3,3,3-trifluoropropyl)thiazole-5-carboxamide

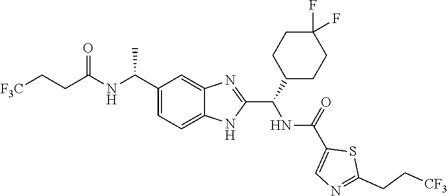 [SMILES: C[C@@H](NC(=O)CCC(F)(F)F)c1ccc2[nH]c([C@@H](NC(=O)c3cnc(CCC(F)(F)F)s3)C3CCC(F)(F)CC3)nc2c1]

The title compound was prepared as described for the synthesis of Example 188, using 2-(3,3,3-trifluoropropyl)thiazole-5-carboxylic acid in place of 4-isopropyl-1,2,3-thiadiazole-5-carboxylic acid. Purification via preparative HPLC using a Boston Prime C18, 150×30 mm×5 μm column (45-75% (v/v) CH₃CN in H₂O with 0.04% NH₄OH and 10 mM NH₄HCO₃) followed by lyophilization yielded the title compound. ¹H NMR (400 MHz, DMSO-d₆) δ 12.39 (s, 1H), 9.26-9.09 (m, 1H), 8.56-8.40 (m, 2H), 7.56-7.47 (m, 1H), 7.43-7.33 (m, 1H), 7.19-7.05 (m, 1H), 5.18-5.08 (m, 1H), 5.06-4.96 (m, 1H), 3.30-3.23 (m, 2H), 2.88-2.71 (m, 2H), 2.49-2.33 (m, 4H), 2.32-2.21 (m, 1H), 2.13-1.92 (m, 3H), 1.91-1.68 (m, 2H), 1.61-1.48 (m, 1H), 1.44-1.23 (m, 5H). MS (ESI) m/z: [M+H]⁺ Found 640.3.

### Example 223

### 5-Methyl-N—((S*)-4,4,4-trifluoro-3,3-dimethyl-1-(5-((R)-1-(4,4,4-trifluorobutanamido)ethyl)-1H-benzo[d]imidazol-2-yl)butyl)-1-(2,2,2-trifluoroethyl)-1H-pyrazole-4-carboxamide

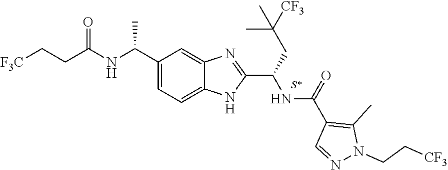 [SMILES: Cc1c(C(=O)N[C@@H](CC(C)(C)C(F)(F)F)c2nc3cc([C@@H](C)NC(=O)CCC(F)(F)F)ccc3[nH]2)cnn1CCC(F)(F)F]

The title compound was prepared as described for the synthesis of Example 188, using N—((R)-1-(2-((S*)-1-amino-4,4,4-trifluoro-3,3-dimethylbutyl)-1H-benzo[d]imidazol-5-yl)ethyl)-4,4,4-trifluorobutanamide (Intermediate 281) in place of N—((R)-1-(2-((S)-amino(4,4-difluorocyclohexyl)methyl)-1H-benzo[d]imidazol-5-yl)ethyl)-4,4,4-trifluorobutanamide and 5-methyl-1-(2,2,2-trifluoroethyl)-1H-pyrazole-4-carboxylic acid in place of 4-isopropyl-1,2,3-thiadiazole-5-carboxylic acid. Purification via preparative HPLC (C18, 50×250 mm, 10 μm column, ACN/H₂O with 0.05% TFA) followed by neutralization using a Silicycle® SiliaPrep™ Carbonate plug eluting with methanol and concentration yielded the title compound. ¹H NMR (500 MHz, CD₃OD) δ 7.93 (s, 1H), 7.16-7.34 (m, 2H), 7.15-7.09 (m, 1H), 5.56-5.46 (m, 1H), 5.08-4.93 (m, 1H), 4.85 (q, J=8.7 Hz, 2H), 2.31-2.30 (m, 1H), 2.50-2.30 (m, 7H), 2.27-2.20 (m, 1H), 1.39 (d, J=7.0 Hz, 3H), 1.14 (s, 3H), 1.10 (s, 3H). MS (ESI) m/z: [M+H]⁺ Found 629.1.

### Example 224

### N—((S)-(4,4-Difluorocyclohexyl)(5-((R)-1-(4,4,4-trifluorobutanamido)ethyl)-1H-benzo[d]imidazol-2-yl)methyl)-2-(3,3,3-trifluoropropyl)thiazole-4-carboxamide

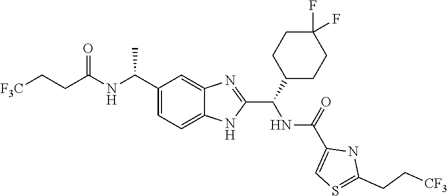

The title compound was prepared as described for the synthesis of Example 188, using 2-(3,3,3-trifluoropropyl)thiazole-4-carboxylic acid (Intermediate 297) in place of 4-isopropyl-1,2,3-thiadiazole-5-carboxylic acid. Purification via preparative HPLC using a Boston Prime C18, 150×25 mm×5 μm column (45-75% (v/v) CH₃CN in H₂O with 0.04% NH₄OH and 10 mM NH₄HCO₃) followed by lyophilization provided the title compound. ¹H NMR (400 MHz, DMSO-d₆) δ 12.43 (br. s, 1H), 8.64-8.39 (m, 2H), 8.25 (s, 1H), 7.60-7.48 (m, 1H), 7.46-7.36 (m, 1H), 7.18-7.08 (m, 1H), 5.28-5.14 (m, 1H), 5.07-4.95 (m, 1H), 3.33-3.27 (m, 2H), 2.95-2.80 (m, 2H), 2.48-2.35 (m, 4H), 2.29-2.16 (m, 1H), 2.11-1.94 (m, 2H), 1.93-1.65 (m, 3H), 1.58-1.46 (m, 1H), 1.38 (d, J=6.8 Hz, 3H), 1.35-1.19 (m, 2H). MS (ESI) m/z: [M+H]⁺ Found 640.3.

### Example 225

### N—((S)-(4,4-Difluorocyclohexyl)(5-((R)-1-(4,4,4-trifluorobutanamido)ethyl)-1H-benzo[d]imidazol-2-yl)methyl)-2-(3,3,3-trifluoropropyl)oxazole-4-carboxamide

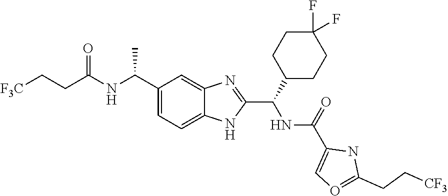

The title compound was prepared as described for the synthesis of Example 188, using 2-(3,3,3-trifluoropropyl)oxazole-4-carboxylic acid (Intermediate 300) in place of 4-isopropyl-1,2,3-thiadiazole-5-carboxylic acid. Purification via preparative HPLC with a Boston Prime C18, 150×25 mm×5 μm column (50-80% (v/v) CH₃CN in H₂O with 0.04% NH₄OH and 10 mM NH₄HCO₃) followed by lyophilization provided the title compound. ¹H NMR (400 MHz, DMSO-d₆) δ 12.41 (s, 1H), 8.65 (s, 1H), 8.53-8.43 (m, 1H), 8.36 (d, J=9.0 Hz, 1H), 7.57-7.50 (m, 1H), 7.47-7.38 (m, 1H), 7.19-7.09 (m, 1H), 5.23-5.13 (m, 1H), 5.07-4.94 (m, 1H), 3.11 (t, J=7.7 Hz, 2H), 2.88-2.75 (m, 2H), 2.49-2.36 (m, 4H), 2.25-2.14 (m, 1H), 2.10-1.92 (m, 2H), 1.91-1.68 (m, 3H), 1.55-1.45 (m, 1H), 1.38 (d, J=7.0 Hz, 3H), 1.34-1.18 (m, 2H). MS (ESI) m/z: [M+H]⁺ Found 624.3.

### Example 226

### N—((S)-(4,4-Difluorocyclohexyl)(5-((R)-1-(4,4,4-trifluorobutanamido)ethyl)-1H-benzo[d]imidazol-2-yl)methyl)-1-(3,3,3-trifluoropropyl)-1H-imidazole-4-carboxamide

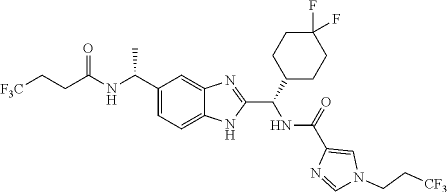 [SMILES: C[C@@H](NC(=O)CCC(F)(F)F)c1ccc2[nH]c([C@@H](NC(=O)c3cn(CCC(F)(F)F)cn3)C3CCC(F)(F)CC3)nc2c1]

The title compound was prepared as described for the synthesis of Example 188, using 1-(3,3,3-trifluoropropyl)-1H-imidazole-4-carboxylic acid (Intermediate 302) in place of 4-isopropyl-1,2,3-thiadiazole-5-carboxylic acid. Purification via preparative HPLC with a Xtimate C18, 150×30 mm×5 μm column (35-65% (v/v) CH₃CN in H₂O with 0.04% NH₄OH and 10 mM NH₄HCO₃) followed by lyophilization provided the title compound. ¹H NMR (400 MHz, DMSO-d₆) δ 12.40 (s, 1H), 8.50-8.38 (m, 1H), 8.18-8.10 (m, 1H), 7.89-7.81 (m, 2H), 7.56-7.50 (m, 1H), 7.45-7.37 (m, 1H), 7.21-7.08 (m, 1H), 5.22-5.13 (m, 1H), 5.07-4.96 (m, 1H), 4.30 (t, J=6.9 Hz, 2H), 2.99-2.82 (m, 2H), 2.48-2.36 (m, 4H), 2.20-2.07 (m, 1H), 2.06-1.91 (m, 2H), 1.88-1.63 (m, 3H), 1.56-1.44 (m, 1H), 1.38 (d, J=6.8 Hz, 3H), 1.33-1.16 (m, 2H). MS (ESI) m/z: [M+H]⁺ Found 623.3.

### Example 227

### N—((S)-(4,4-Difluorocyclohexyl)(5-((R)-1-(4,4,4-trifluorobutanamido)ethyl)-1H-benzo[d]imidazol-2-yl)methyl)-1-(3,3,3-trifluoropropyl)-1H-imidazole-2-carboxamide

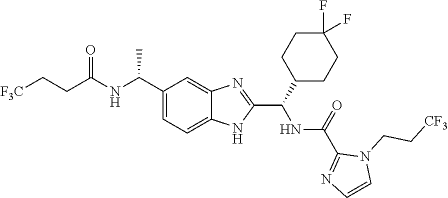 [SMILES: C[C@@H](NC(=O)CCC(F)(F)F)c1ccc2[nH]c([C@@H](NC(=O)c3nccn3CCC(F)(F)F)C3CCC(F)(F)CC3)nc2c1]

The title compound was prepared as described for the synthesis of Example 188, using 1-(3,3,3-trifluoropropyl)-1H-imidazole-2-carboxylic acid (Intermediate 304) in place of 4-isopropyl-1,2,3-thiadiazole-5-carboxylic acid. Purification via preparative HPLC (column: Phenomenex Gemini-NX 150×30 mm×5 μm, mobile phase: 41-71% ACN/water (with 0.04% NH₄OH and 10 mM NH₄HCO₃)) followed by lyophilization provided the title compound. ¹H NMR (400 MHz, DMSO-d₆) δ 12.40 (br s, 1H), 8.66 (d, J=9.3 Hz, 1H), 8.51-8.42 (m, 1H), 7.59-7.35 (m, 3H), 7.17-7.10 (m, 1H), 7.08 (d, J=1.2 Hz, 1H), 5.21-5.14 (m, 1H), 5.06-4.96 (m, 1H), 4.74-4.57 (m, 2H), 2.91-2.76 (m, 2H), 2.48-2.36 (m, 4H), 2.25-2.12 (m, 1H), 2.10-1.94 (m, 2H), 1.92-1.66 (m, 3H), 1.59-1.48 (m, 1H), 1.39 (d, J=7.0 Hz, 3H), 1.35-1.20 (m, 2H). MS (ESI) m/z: [M+H]⁺ Found 623.3.

### Example 228

### N—((S)-(4,4-Difluorocyclohexyl)(5-((R)-1-(4,4,4-trifluorobutanamido)ethyl)-1H-benzo[d]imidazol-2-yl)methyl)-1-(3,3,3-trifluoropropyl)-1H-1,2,4-triazole-3-carboxamide

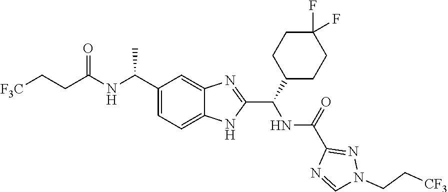 [SMILES: C[C@@H](NC(=O)CCC(F)(F)F)c1ccc2[nH]c([C@@H](NC(=O)c3ncn(CCC(F)(F)F)n3)C3CCC(F)(F)CC3)nc2c1]

The title compound was prepared as described for the synthesis of Example 188, using 1-(3,3,3-trifluoropropyl)-1H-1,2,4-triazole-3-carboxylic acid (Intermediate 308) in place of 4-isopropyl-1,2,3-thiadiazole-5-carboxylic acid. Purification via preparative HPLC with an Agela Durashell C18, 150×30 mm×5 μm column (32-62% (v/v) CH₃CN in H₂O with 0.05% NH₄OH) followed by lyophilization provided the title compound. ¹H NMR (400 MHz, DMSO-d₆) δ 12.41 (s, 1H), 8.76 (s, 1H), 8.68-8.61 (m, 1H), 8.51-8.43 (m, 1H), 7.57-7.49 (m, 1H), 7.46-7.37 (m, 1H), 7.18-7.09 (m, 1H), 5.26-5.14 (m, 1H), 5.08-4.95 (m, 1H), 4.61-4.51 (m, 2H), 3.04-2.89 (m, 2H), 2.48-2.31 (m, 4H), 2.26-2.16 (m, 1H), 2.08-1.94 (m, 2H), 1.92-1.66 (m, 3H), 1.57-1.46 (m, 1H), 1.38 (d, J=7.0 Hz, 3H), 1.35-1.20 (m, 2H). MS (ESI) m/z: [M+H]⁺ Found 624.3.

### Example 229

### N—((S)-4,4,4-Trifluoro-3,3-dimethyl-1-(6-((S*)-1-(4,4,4-trifluorobutanamido)ethyl)-1H-benzo[d]imidazol-2-yl)butyl)-1-(3,3,3-trifluoropropyl)-1H-pyrazole-5-carboxamide

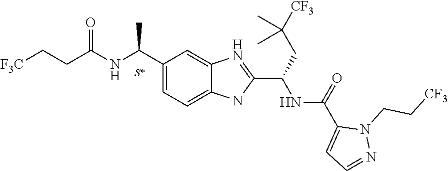

### Example 230

### N—((S)-4,4,4-Trifluoro-3,3-dimethyl-1-(6-((R*)-1-(4,4,4-trifluorobutanamido)ethyl)-1H-benzo[d]imidazol-2-yl)butyl)-1-(3,3,3-trifluoropropyl)-1H-pyrazole-5-carboxamide

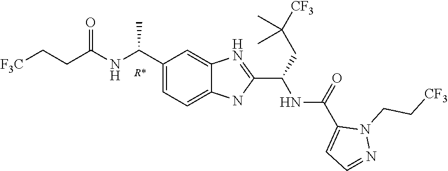

To a solution of 4,4,4-trifluorobutanoic acid (154 mg, 1.08 mmol), HOAt (200 mg, 1.47 mmol), and dichloromethane (20 mL) at 0° C. was added EDCI (210 mg, 1.10 mmol). The resultant mixture was stirred at 0° C. for 30 min. A solution of N-((1S)-1-(5-(1-aminoethyl)-1H-benzo[d]imidazol-2-yl)-4,4,4-trifluoro-3,3-dimethylbutyl)-1-(3,3,3-trifluoropropyl)-1H-pyrazole-5-carboxamide (500.0 mg, 0.991 mmol, Intermediate 314) and DIPEA (690 μL, 3.93 mmol) was added and the reaction mixture was stirred at 0° C. for 2 h. The reaction was quenched with 10 mL of saturated aqueous NH₄Cl. The layers were separated and the aqueous fraction was extracted with EtOAc (20 mL×3). The combined organic extracts were washed with H₂O, dried over anhydrous Na₂SO₄, filtered, and concentrated to dryness under reduced pressure. Purification by preparative chromatography (43-73%, v/v ACN/water (with 0.04% NH₄OH+10 mM NH₄HCO₃)) afforded a mixture of diastereomers. Purification via SFC (REGIS (s,s) WHELK-01, 250×30 mm, 5 μm column, 50% (v/v) supercritical CO₂ in EtOH and H₂O with 0.1% NH₄OH) followed by lyophilization yielded the title compounds. The first eluting isomer was Example 230 and the second eluting isomer was Example 229. Example 229: ¹H NMR (400 MHz, DMSO-d₆) δ 12.35 (br s, 1H), 9.19 (d, J=8.3 Hz, 1H), 8.56-8.43 (m, 1H), 7.58 (d, J=2.0 Hz, 1H), 7.55-7.50 (m, 1H), 7.43-7.33 (m, 1H), 7.18-7.09 (m, 1H), 7.07-7.01 (m, 1H), 5.54-5.40 (m, 1H), 5.07-4.97 (m, 1H), 4.90-4.80 (m, 1H), 4.76-4.65 (m, 1H), 2.91-2.76 (m, 2H), 2.61-2.54 (m, 1H), 2.50-2.33 (m, 4H), 2.32-2.22 (m, 1H), 1.39 (d, J=7.1 Hz, 3H), 1.17 (s, 3H), 1.14 (s, 3H). MS (ESI) m/z: [M+H]⁺ Found 629.3. Example 230: ¹H NMR (400 MHz, DMSO-d₆) δ 12.36 (br s, 1H), 9.25-9.14 (m, 1H), 8.54-8.45 (m, 1H), 7.58 (d, J=2.0 Hz, 1H), 7.55-7.50 (m, 1H), 7.42-7.34 (m, 1H), 7.17-7.10 (m, 1H), 7.06 (d, J=2.0 Hz, 1H), 5.53-5.42 (m, 1H), 5.07-4.97 (m, 1H), 4.90-4.80 (m, 1H), 4.76-4.66 (m, 1H), 2.91-2.76 (m, 2H), 2.60-2.53 (m, 1H), 2.49-2.33 (m, 4H), 2.33-2.22 (m, 1H), 1.39 (d, J=6.8 Hz, 3H), 1.17 (s, 3H), 1.14 (s, 3H). MS (ESI) m/z: [M+H]⁺ Found 629.3.

### Example 231

### 1-Isopropyl-N—((S)-4,4,4-trifluoro-3,3-dimethyl-1-(6-((S*)-1-(4,4,4-trifluorobutanamido)ethyl)-1H-benzo[d]imidazol-2-yl)butyl)-1H-pyrazole-5-carboxamide

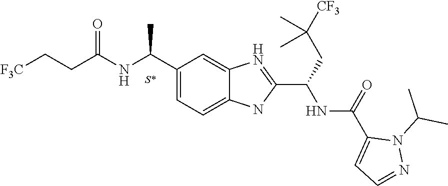

### Example 232

### 1-Isopropyl-N—((S)-4,4,4-trifluoro-3,3-dimethyl-1-(6-((R*)-1-(4,4,4-trifluorobutanamido)ethyl)-1H-benzo[d]imidazol-2-yl)butyl)-1H-pyrazole-5-carboxamide

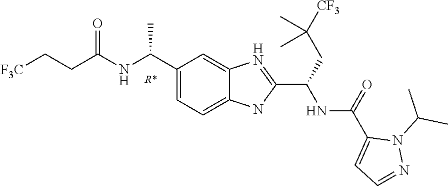

The title compounds were prepared as described for the synthesis of Example 229, using N-((1S)-1-(5-(1-aminoethyl)-1H-benzo[d]imidazol-2-yl)-4,4,4-trifluoro-3,3-dimethylbutyl)-1-isopropyl-1H-pyrazole-5-carboxamide (Intermediate 316) in place of N-((1S)-1-(5-(1-aminoethyl)-1H-benzo[d]imidazol-2-yl)-4,4,4-trifluoro-3,3-dimethylbutyl)-1-(3,3,3-trifluoropropyl)-1H-pyrazole-5-carboxamide. Separation of the diastereomers was accomplished by SFC (REGIS (s,s) WHELK-01 (250×30 mm, 5 μm) column (7:3 (v/v) EtOH (with 0.1% aqueous NH₄OH))/supercritical CO₂)) followed by lyophilization to afford the title compounds. The first eluting isomer was Example 232 and the second eluting isomer was Example 231. Example 231: ¹H NMR (400 MHz, DMSO-d₆) δ 12.35 (br s, 1H), 9.08 (d, J=8.5 Hz, 1H), 8.55-8.41 (m, 1H), 7.55-7.47 (m, 2H), 7.43-7.34 (m, 1H), 7.17-7.08 (m, 1H), 6.98-6.91 (m, 1H), 5.57-5.37 (m, 2H), 5.08-4.97 (m, 1H), 2.59-2.53 (m, 1H), 2.49-2.33 (m, 4H), 2.33-2.22 (m, 1H), 1.42-1.33 (m, 9H), 1.18 (s, 3H), 1.14 (s, 3H). MS (ESI) m/z: [M+H]⁺ Found 575.3. Example 232: ¹H NMR (400 MHz, DMSO-d₆) δ 12.32 (br s, 1H), 9.06 (d, J=8.4 Hz, 1H), 8.60-8.40 (m, 1H), 7.62-7.48 (m, 2H), 7.43-7.34 (m, 1H), 7.20-7.07 (m, 1H), 6.96-6.89 (m, 1H), 5.58-5.39 (m, 2H), 5.09-4.94 (m, 1H), 2.58-2.52 (m, 1H), 2.50-2.32 (m, 4H), 2.32-2.23 (m, 1H), 1.43-1.34 (m, 9H), 1.18 (s, 3H), 1.14 (s, 3H). MS (ESI) m/z: [M+H]⁺ Found 575.3.

### Example 233

### N—((S)-1-(6-((R)-Cyclopropyl(2-(3,3-difluorocyclobutyl)acetamido)methyl)-1H-benzo[d]imidazol-2-yl)-4,4-difluoro-3,3-dimethylbutyl)-4-methyl-1,2,5-oxadiazole-3-carboxamide

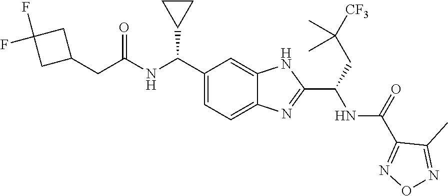 [SMILES: Cc1nonc1C(=O)N[C@@H](CC(C)(C)C(F)(F)F)c1nc2ccc([C@H](NC(=O)CC3CC(F)(F)C3)C3CC3)cc2[nH]1]

EDCI (82 mg, 0.43 mmol) was added to a solution of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide (65 mg, 0.14 mmol, Intermediate 361), 4-methyl-1,2,5-oxadiazole-3-carboxylic acid (37 mg, 0.29 mmol), HOBt (39 mg, 0.29 mmol), and DIPEA (0.1 mL, 0.6 mmol) in CH₂Cl₂ (6 mL), and the mixture stirred overnight at rt. The mixture was then diluted with water (20 mL) and CH₂Cl₂ (20 mL), the layers were separated, and the aqueous phase was extracted with CH₂Cl₂ (3×50 mL). The combined organic layers were washed with water (20 mL) and brine (20 mL), dried over anhydrous Na₂SO₄, filtered, and concentrated to dryness under reduced pressure. The crude product was purified twice by silica gel chromatography (0-10% MeOH/CH₂Cl₂), and then further purified via basic preparative HPLC (Boston Prime, 5 μm, C18, 150×30 mm column, 45-75% MeCN/water (with 0.05% NH₄OH)), followed by SFC using a chiral stationary phase (DAICEL CHIRALPAK AD, 10 μm, 250×30 mm, 75% CO₂ in EtOH (0.1% NH₄OH)). The product containing fractions were diluted with water, frozen, and lyophilized to afford the title compound as a white solid. ¹H NMR (400 MHz, DMSO-d₆) δ 12.24 (s, 1H), 9.79-9.71 (m, 1H), 8.47 (dd, J=8.8, 12.0 Hz, 1H), 7.58-7.49 (m, 1H), 7.42-7.37 (m, 1H), 7.19-7.12 (m, 1H), 6.00-5.68 (m, 1H), 5.48-5.39 (m, 1H), 4.39-4.30 (m, 1H), 2.71-2.57 (m, 2H), 2.52 (s, 3H), 2.48-2.42 (m, 1H), 2.40-2.21 (m, 5H), 2.19-2.10 (m, 1H), 1.21-1.11 (m, 1H), 1.04 (s, 3H), 0.98 (s, 3H), 0.54-0.43 (m, 2H), 0.36-0.27 (m, 2H). MS (ESI) m/z: [M+H]⁺ Found 565.2.

### Example 234

### N—((S)-1-(6-((R)-Cyclopropyl(2-(3,3-difluorocyclobutyl)acetamido)methyl)-1H-benzo[d]imidazol-2-yl)-4,4-difluoro-3,3-dimethylbutyl)-1-isopropyl-1H-pyrazole-5-carboxamide

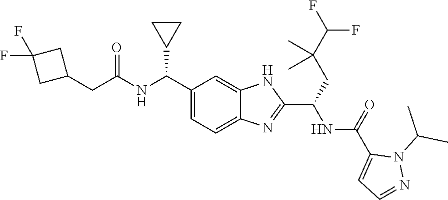 [SMILES: CC(C)n1nccc1C(=O)N[C@@H](CC(C)(C)C(F)F)c1nc2ccc([C@H](NC(=O)CC3CC(F)(F)C3)C3CC3)cc2[nH]1]

DIPEA (0.03 mL, 0.18 mmol) was added to a solution of 1-isopropyl-1H-pyrazole-5-carboxylic acid (14 mg, 0.088 mmol) and T3P© (0.17 mL, 0.13 mmol, 50% in THF) in DCM (1 mL) and was stirred for 0.5 h at 30° C. N—((R)-(2-((S)-1-Amino-4,4-difluoro-3,3-dimethylbutyl)-1H-benzo[d]imidazol-6-yl)(cyclopropyl)methyl)-2-(3,3-difluorocyclobutyl)acetamide (20 mg, 0.044 mmol, Intermediate 361) was added and stirring continued overnight. The reaction mixture was then diluted with water (20 mL) and DCM (20 mL), and the phases separated. The aqueous phase was further extracted with DCM (3×50 mL), and the combined washed with water (20 mL) and brine (20 mL), dried over anhydrous Na₂SO₄, filtered, and concentrated to dryness under reduced pressure to afford the crude product, which was purified by silica gel chromatography (0-10% MeOH/DCM). The product containing fractions were concentrated under reduced pressure, suspended in water, frozen, and lyophilized to afford the title compound as a white solid. ¹H NMR (400 MHz, DMSO-d₆) δ 12.26 (s, 1H), 8.99 (dd, J=4.4, 8.4 Hz, 1H), 8.48 (dd, J=8.4, 11.6 Hz, 1H), 7.58-7.46 (m, 2H), 7.38 (d, J=10.4 Hz, 1H), 7.15 (dd, J=5.2, 7.2 Hz, 1H), 6.92 (d, J=2.0 Hz, 1H), 5.99-5.64 (m, 1H), 5.54-5.39 (m, 2H), 4.40-4.29 (m, 1H), 2.69-2.59 (m, 1H), 2.39-2.26 (m, 5H), 2.16-2.06 (m, 1H), 1.42-1.32 (m, 6H), 1.29-1.21 (m, 2H), 1.19-1.11 (m, 1H), 1.06-0.93 (m, 6H), 0.55-0.42 (m, 2H), 0.37-0.26 (m, 2H). MS (ESI) m/z: [M+H]⁺ Found 591.2.

### Example 235

### N—((S)-1-(6-((R)-Cyclopropyl(2-(3,3-difluorocyclobutyl)acetamido)methyl)-1H-benzo[d]imidazol-2-yl)-2-((R)-3,3-difluorocyclopentyl)ethyl)-4-methyl-1,2,5-oxadiazole-3-carboxamide

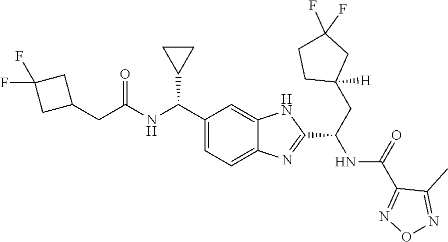 [SMILES: Cc1nonc1C(=O)N[C@@H](C[C@H]1CCC(F)(F)C1)c1nc2ccc([C@H](NC(=O)CC3CC(F)(F)C3)C3CC3)cc2[nH]1]

The title compound was prepared as described for the synthesis of Example 233, using N—((R)-(2-((S)-1-amino-2-((R)-3,3-difluorocyclopentyl)ethyl)-1H-benzo[d]imidazol-6-yl)(cyclopropyl)methyl)-2-(3,3-difluorocyclobutyl)acetamide (Intermediate 363) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (0-8% MeOH/DCM) to afford the title compound, which was further purified by SFC using a chiral stationary phase (Phenomenex-Cellulose-2, 5 μm, 250×30 mm, 80% CO₂ in MeOH (0.1% NH₄OH)). The product containing fractions were diluted with water, frozen, and lyophilized to afford the title compound as an off-white solid. ¹H NMR (400 MHz, DMSO-d₆) δ 12.27 (s, 1H), 9.65-9.62 (m, 1H), 8.50-8.45 (m, 1H), 7.57-7.47 (m, 1H), 7.42-7.36 (m, 1H), 7.18-7.12 (m, 1H), 5.30-5.21 (m, 1H), 4.38-4.29 (m, 1H), 2.71-2.56 (m, 3H), 2.43-2.07 (m, 11H), 2.05-1.90 (m, 2H), 1.88-1.69 (m, 1H), 1.59-1.40 (m, 1H), 1.28-1.01 (m, 2H), 0.54-0.39 (m, 2H), 0.32-0.30 (m, 2H). MS (ESI) m/z: [M+H]⁺ Found 577.1.

### Example 236

### N—((S)-1-(6-((R)-Cyclopropyl(2-(3,3-difluorocyclobutyl)acetamido)methyl)-1H-benzo[d]imidazol-2-yl)-2-((R)-3,3-difluorocyclopentyl)ethyl)-1-isopropyl-1H-pyrazole-5-carboxamide

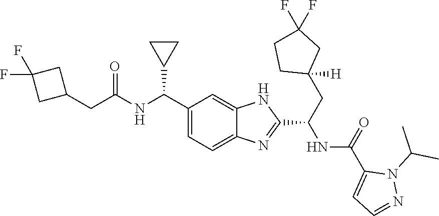 [SMILES: CC(C)n1nccc1C(=O)N[C@@H](C[C@H]1CCC(F)(F)C1)c1nc2ccc([C@H](NC(=O)CC3CC(F)(F)C3)C3CC3)cc2[nH]1]

The title compound was prepared as described for the synthesis of Example 233, using N—((R)-(2-((S)-1-amino-2-((R)-3,3-difluorocyclopentyl)ethyl)-1H-benzo[d]imidazol-6-yl)(cyclopropyl)methyl)-2-(3,3-difluorocyclobutyl)acetamide (Intermediate 363) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 1-isopropyl-1H-pyrazole-5-carboxylic acid in place of 4-methyl-1,2,5-oxadiazole-3-carboxylic acid. The crude product was purified by silica gel chromatography (0-2% MeOH/DCM) followed by SFC using a chiral stationary phase (Phenomenex-Cellulose-2, 10 μm, 250×30 mm, 80% CO₂ in MeOH (0.1% NH₄OH)). The product containing fractions were diluted with water, frozen, and lyophilized to afford the title compound as a white solid. ¹H NMR (400 MHz, DMSO-d₆) δ 12.27 (s, 1H), 8.91-8.89 (m, 1H), 8.52-8.44 (m, 1H), 7.56-7.47 (m, 2H), 7.44-7.36 (m, 1H), 7.19-7.12 (m, 1H), 6.95-6.94 (m, 1H), 5.53-5.43 (m, 1H), 5.31-5.22 (m, 1H), 4.46-4.24 (m, 1H), 2.62-2.51 (m, 3H), 2.36-2.33 (m, 3H), 2.31-2.26 (m, 2H), 2.22-2.03 (m, 5H), 1.97-1.96 (m, 1H), 1.87-1.71 (m, 1H), 1.57-1.44 (m, 1H), 1.38-1.36 (m, 6H), 1.25-1.09 (m, 1H), 0.49-0.47 (m, 2H), 0.35-0.28 (m, 2H). MS (ESI) m/z: [M+H]⁺ Found 603.3.

### Example 237

### N—((S)-(6-((R)-Cyclopropyl(2-(3,3-difluorocyclobutyl)acetamido)methyl)-1H-benzo[d]imidazol-2-yl)((1R,3s,5S)-6,6-difluorobicyclo[3.1.0]hexan-3-yl)methyl)-4-methyl-1,2,5-oxadiazole-3-carboxamide

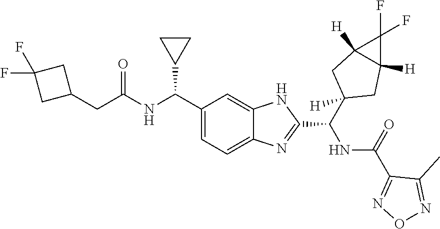 [SMILES: Cc1nonc1C(=O)N[C@H](c1nc2ccc([C@H](NC(=O)CC3CC(F)(F)C3)C3CC3)cc2[nH]1)[C@@H]1C[C@@H]2[C@H](C1)C2(F)F]

The title compound was prepared as described for the synthesis of Example 233, using N—((R)-(2-((S)-amino((1R,3s,5S)-6,6-difluorobicyclo[3.1.0]hexan-3-yl)methyl)-1H-benzo[d]imidazol-6-yl)(cyclopropyl)methyl)-2-(3,3-difluorocyclobutyl)acetamide (Intermediate 365) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (50-100% EtOAc/petroleum ether) followed by SFC using a chiral stationary phase (DAICEL CHIRALCEL OD-H, 5 μm, 250×30 mm, 80% CO₂ in EtOH (0.1% NH₄OH)). The product containing fractions were diluted with water, frozen, and lyophilized to afford the title compound as a white solid. ¹H NMR (400 MHz, DMSO-d₆) δ12.28 (s, 1H), 9.67 (dd, J=3.6, 8.4 Hz, 1H), 8.47 (dd, J=8.4, 14.4 Hz, 1H), 7.60-7.49 (m, 1H), 7.44-7.36 (m, 1H), 7.15 (dd, J=4.4, 7.6 Hz, 1H), 5.15 (t, J=8.4 Hz, 1H), 4.45-4.15 (m, 1H), 2.86-2.71 (m, 1H), 2.70-2.56 (m, 2H), 2.49 (s, 3H), 2.42-2.23 (m, 5H), 2.20-2.06 (m, 3H), 2.03-1.80 (m, 3H), 1.31-1.07 (m, 1H), 0.50-0.46 (m, 2H), 0.33-0.30 (m, 2H). MS (ESI) m/z: [M+H]⁺ Found 575.4.

### Example 238

### N—((S)-(6-((R)-Cyclopropyl(2-(3,3-difluorocyclobutyl)acetamido)methyl)-1H-benzo[d]imidazol-2-yl)((1R,3s,5S)-6,6-difluorobicyclo[3.1.0]hexan-3-yl)methyl)-1-isopropyl-1H-pyrazole-5-carboxamide

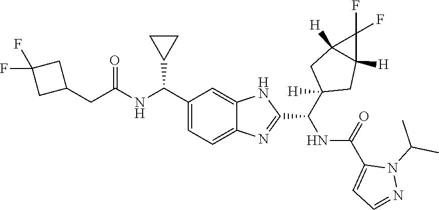 [SMILES: CC(C)n1nccc1C(=O)N[C@H](c1nc2ccc([C@H](NC(=O)CC3CC(F)(F)C3)C3CC3)cc2[nH]1)[C@@H]1C[C@@H]2[C@H](C1)C2(F)F]

The title compound was prepared as described for the synthesis of Example 234, using N—((R)-(2-((S)-amino((1R,3s,5S)-6,6-difluorobicyclo[3.1.0]hexan-3-yl)methyl)-1H-benzo[d]imidazol-6-yl)(cyclopropyl)methyl)-2-(3,3-difluorocyclobutyl)acetamide (Intermediate 365) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (0-5% MeOH, DCM) followed by SFC using a chiral stationary phase (Phenomenex-Cellulose-2, 5 μm, 250×30 mm, 75% CO₂ in MeOH (0.1% NH₄OH)). The product containing fractions were diluted with water, frozen, and lyophilized to afford the title compound as a white solid. ¹H NMR (400 MHz, DMSO-d₆) δ 8.97 (s, 1H), 8.47 (d, J=8.4 Hz, 1H), 7.54-7.40 (m, 3H), 7.15-7.13 (m, 1H), 6.96 (d, J=2.0 Hz, 1H), 5.53-5.28 (m, 1H), 5.10 (d, J=9.2 Hz, 1H), 4.33 (t, J=8.4 Hz, 1H), 2.81-2.73 (m, 1H), 2.69-2.57 (m, 2H), 2.43-2.25 (m, 5H), 2.23-2.17 (m, 1H), 2.12-2.08 (m, 2H), 2.02-1.78 (m, 3H), 1.38-1.33 (m, 6H), 1.27-1.08 (m, 2H), 0.56-0.41 (m, 2H), 0.38-0.24 (m, 2H). MS (ESI) m/z: [M+H]⁺ Found 601.3.

### Example 239

### 4-Cyclopropyl-N—((S)-(6-((R)-cyclopropyl(2-(3,3-difluorocyclobutyl)acetamido)methyl)-1H-benzo[d]imidazol-2-yl)((1R,3s,5S)-6,6-difluorobicyclo[3.1.0]hexan-3-yl)methyl)-1,2,5-oxadiazole-3-carboxamide

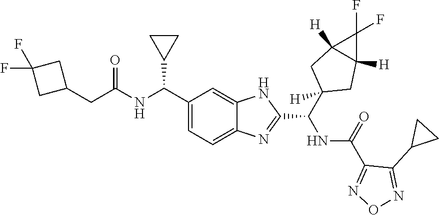 [SMILES: O=C(CC1CC(F)(F)C1)N[C@@H](c1ccc2nc([C@@H](NC(=O)c3nonc3C3CC3)[C@@H]3C[C@@H]4[C@H](C3)C4(F)F)[nH]c2c1)C1CC1]

The title compound was prepared as described for the synthesis of Example 233, using N—((R)-(2-((S)-amino((1R,3s,5S)-6,6-difluorobicyclo[3.1.0]hexan-3-yl)methyl)-1H-benzo[d]imidazol-6-yl)(cyclopropyl)methyl)-2-(3,3-difluorocyclobutyl)acetamide hydrogen chloride (Intermediate 283) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 4-cyclopropyl-1,2,5-oxadiazole-3-carboxylic acid (Intermediate 78) in place of 4-methyl-1,2,5-oxadiazole-3-carboxylic acid. The crude product was purified by silica gel chromatography (0-1% MeOH/DCM) to afford the title compound, which was suspended in water, frozen, and lyophilized to afford the title compound as a white solid. ¹H NMR (400 MHz, DMSO-d₆) δ 12.35 (s, 1H), 9.74 (d, J=8.4 Hz, 1H), 8.53-8.51 (m, 1H), 7.49-7.47 (m, 2H), 7.18-7.16 (m, 1H), 5.17 (t, J=8.8 Hz, 1H), 4.33 (t, J=8.4 Hz, 1H), 2.84-2.55 (m, 3H), 2.43-2.26 (m, 6H), 2.24-2.07 (m, 3H), 2.04-1.81 (m, 3H), 1.21-1.10 (m, 3H), 0.99-0.97 (m, 2H), 0.55-0.43 (m, 2H), 0.32-0.31 (m, 2H). MS (ESI) m/z: [M+H]⁺ Found 601.1.

### Example 240

### N—((S)-(6-((R)-Cyclopropyl(2-(3,3-difluorocyclobutyl)acetamido)methyl)-1H-benzo[d]imidazol-2-yl)((1R,3r,5S)-6,6-difluorobicyclo[3.1.0]hexan-3-yl)methyl)-4-methyl-1,2,5-oxadiazole-3-carboxamide

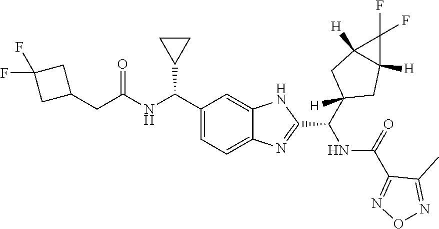 [SMILES: Cc1nonc1C(=O)N[C@H](c1nc2ccc([C@H](NC(=O)CC3CC(F)(F)C3)C3CC3)cc2[nH]1)[C@H]1C[C@@H]2[C@H](C1)C2(F)F]

The title compound was prepared as described for the synthesis of Example 233, using N—((R)-(2-((S)-amino((1R,3r,5S)-6,6-difluorobicyclo[3.1.0]hexan-3-yl)methyl)-1H-benzo[d]imidazol-6-yl)(cyclopropyl)methyl)-2-(3,3-difluorocyclobutyl)acetamide-hydrogen chloride (Intermediate 367) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (0-2% MeOH/DCM) to afford the title compound. The product containing fractions were diluted with water, frozen, and lyophilized to afford the title compound as a white solid. ¹H NMR (400 MHz, DMSO-d₆) δ 12.30 (s, 1H), 9.69-9.62 (m, 1H), 8.50 (d, J=8.4 Hz, 1H), 7.55-7.38 (m, 2H), 7.18-7.16 (m, 1H), 5.09 (t, J=9.2 Hz, 1H), 4.33 (t, J=8.4 Hz, 1H), 3.36-3.21 (m, 1H), 2.74-2.57 (m, 2H), 2.48 (s, 3H), 2.38-2.14 (m, 7H), 2.12-1.98 (m, 1H), 1.65-1.59 (m, 1H), 1.52-1.46 (m, 1H), 1.28-1.20 (m, 1H), 1.20-1.12 (m, 1H), 0.55-0.40 (m, 2H), 0.37-0.23 (m, 2H). MS (ESI) m/z: [M+H]⁺ Found 575.2.

### Example 241

### N—((S)-(6-((R)-Cyclopropyl(2-(3,3-difluorocyclobutyl)acetamido)methyl)-1H-benzo[d]imidazol-2-yl)((1R,3r,5S)-6,6-difluorobicyclo[3.1.0]hexan-3-yl)methyl)-1-isopropyl-1H-pyrazole-5-carboxamide

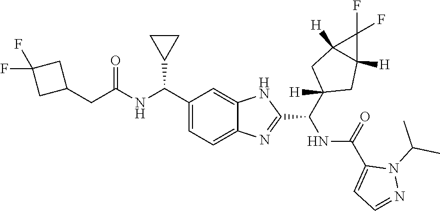 [SMILES: CC(C)n1nccc1C(=O)N[C@H](c1nc2ccc([C@H](NC(=O)CC3CC(F)(F)C3)C3CC3)cc2[nH]1)[C@H]1C[C@@H]2[C@H](C1)C2(F)F]

The title compound was prepared as described for the synthesis of Example 234, using N—((R)-(2-((S)-amino((1R,3r,5S)-6,6-difluorobicyclo[3.1.0]hexan-3-yl)methyl)-1H-benzo[d]imidazol-6-yl)(cyclopropyl)methyl)-2-(3,3-difluorocyclobutyl)acetamide-hydrogen chloride (Intermediate 367) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by preparative silica gel TLC (5% EtOAc/petroleum ether) to afford the title compound, which was diluted with water, frozen, and lyophilized to afford the title compound as a white solid. ¹H NMR (400 MHz, DMSO-d₆) δ 12.29 (s, 1H), 8.91-8.88 (m, 1H), 8.52-8.46 (m, 1H), 7.56-7.46 (m, 2H), 7.42-7.36 (m, 1H), 7.17-7.13 (m, 1H), 6.89 (d, J=2.0 Hz, 1H), 5.53-5.32 (m, 1H), 5.08 (t, J=9.2 Hz, 1H), 4.35-4.30 (m, 1H), 3.31-3.22 (m, 1H), 2.82-2.56 (m, 2H), 2.42-1.92 (m, 9H), 1.63-1.57 (m, 1H), 1.53-1.41 (m, 1H), 1.37-1.33 (m, 6H), 1.21-1.10 (m, 1H), 0.54-0.41 (m, 2H), 0.31-0.30 (m, 2H). MS (ESI) m/z: [M+H]⁺ Found 601.3.

### Example 242

### N—((S)-1-(6-((R)-Cyclopropyl(2-(3,3-difluorocyclobutyl)acetamido)methyl)-1H-benzo[d]imidazol-2-yl)-2-(1-(trifluoromethyl)cyclopropyl)ethyl)-4-methyl-1,2,5-oxadiazole-3-carboxamide

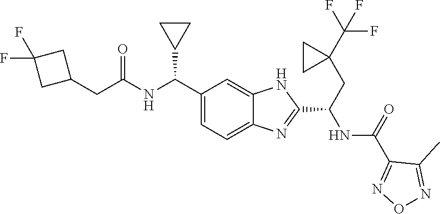 [SMILES: Cc1nonc1C(=O)N[C@@H](CC1(C(F)(F)F)CC1)c1nc2ccc([C@H](NC(=O)CC3CC(F)(F)C3)C3CC3)cc2[nH]1]

The title compound was prepared as described for the synthesis of Example 233, using N—((R)-(2-((S)-1-amino-2-(1-(trifluoromethyl)cyclopropyl)ethyl)-1H-benzo[d]imidazol-6-yl)(cyclopropyl)methyl)-2-(3,3-difluorocyclobutyl)acetamide (Intermediate 369) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by SFC using a chiral stationary phase (DAICEL CHIRALPAK AD, 10 μm, 250×30 mm, 70% CO₂ in EtOH (0.1% NH₄OH)). The product containing fractions were diluted with water, frozen, and lyophilized to afford the title compound as a white solid. ¹H NMR (400 MHz, DMSO-d₆) δ 12.29 (s, 1H), 9.71-9.62 (m, 1H), 8.48 (dd, J=8.4, 12.8 Hz, 1H), 7.60-7.47 (m, 1H), 7.42-7.36 (m, 1H), 7.17 (t, J=7.2 Hz, 1H), 5.57-5.48 (m, 1H), 4.39-4.30 (m, 1H), 2.82-2.72 (m, 1H), 2.71-2.57 (m, 2H), 2.53 (s, 3H), 2.41-2.17 (m, 6H), 1.21-1.05 (m, 2H), 1.00-0.83 (m, 2H), 0.72-0.63 (m, 1H), 0.55-0.41 (m, 2H), 0.38-0.25 (m, 2H). MS (ESI) m/z: [M+H]⁺ Found 581.4.

### Example 243

### N—((S)-1-(6-((R)-Cyclopropyl(2-(3,3-difluorocyclobutyl)acetamido)methyl)-1H-benzo[d]imidazol-2-yl)-2-(1-(trifluoromethyl)cyclopropyl)ethyl)-1-isopropyl-1H-pyrazole-5-carboxamide

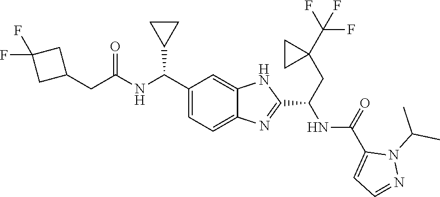 [SMILES: CC(C)n1nccc1C(=O)N[C@@H](CC1(C(F)(F)F)CC1)c1nc2ccc([C@H](NC(=O)CC3CC(F)(F)C3)C3CC3)cc2[nH]1]

The title compound was prepared as described for the synthesis of Example 233, using N—((R)-(2-((S)-1-amino-2-(1-(trifluoromethyl)cyclopropyl)ethyl)-1H-benzo[d]imidazol-6-yl)(cyclopropyl)methyl)-2-(3,3-difluorocyclobutyl)acetamide (Intermediate 369) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 1-isopropyl-1H-pyrazole-5-carboxylic acid in place of 4-methyl-1,2,5-oxadiazole-3-carboxylic acid. The crude product was purified by silica gel chromatography (0-5% MeOH/DCM) followed by basic preparative HPLC (Phenomenex Gemini-NX, 3 μm, C18, 75×30 mm, 40-70% MeCN/water (with 0.05% NH₄OH and 10 mM NH₄HCO₃)). The product containing fractions were diluted with water, frozen, and lyophilized to afford the title compound as white solid. ¹H NMR (400 MHz, DMSO-d₆) δ 12.32 (d, J=3.6 Hz, 1H), 8.95-8.89 (m, 1H), 8.54-8.46 (m, 1H), 7.56-7.48 (m, 2H), 7.39 (d, J=10.4 Hz, 1H), 7.19-7.13 (m, 1H), 6.90 (d, J=1.6 Hz, 1H), 5.54-5.42 (m, 2H), 4.37-4.29 (m, 1H), 2.71-2.57 (m, 3H), 2.40-2.15 (m, 6H), 1.39-1.34 (m, 6H), 1.22-1.03 (m, 2H), 0.97-0.81 (m, 2H), 0.71-0.63 (m, 1H), 0.54-0.43 (m, 2H), 0.34-0.27 (m, 2H). MS (ESI) m/z: [M+H]⁺ Found 607.2.

### Example 244

### N—((S)-(6-((R)-Cyclopropyl(2-(3,3-difluorocyclobutyl)acetamido)methyl)-1H-benzo[d]imidazol-2-yl)((1s,3R)-3-(2,2,2-trifluoroethyl)cyclobutyl)methyl)-4-methyl-1,2,5-oxadiazole-3-carboxamide

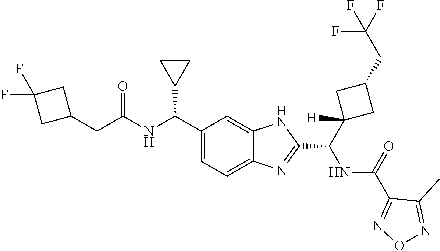 [SMILES: Cc1nonc1C(=O)N[C@H](c1nc2ccc([C@H](NC(=O)CC3CC(F)(F)C3)C3CC3)cc2[nH]1)[C@H]1C[C@@H](CC(F)(F)F)C1]

### Example 245

### N—((S)-(6-((R)-Cyclopropyl(2-(3,3-difluorocyclobutyl)acetamido)methyl)-1H-benzo[d]imidazol-2-yl)((1r,3S)-3-(2,2,2-trifluoroethyl)cyclobutyl)methyl)-4-methyl-1,2,5-oxadiazole-3-carboxamide

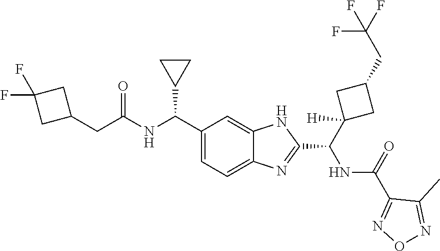 [SMILES: Cc1nonc1C(=O)N[C@H](c1nc2ccc([C@H](NC(=O)CC3CC(F)(F)C3)C3CC3)cc2[nH]1)[C@H]1C[C@H](CC(F)(F)F)C1]

The title compounds were prepared as described for the synthesis of Example 234, using N—((R)-(2-((S)-amino(3-(2,2,2-trifluoroethyl)cyclobutyl)methyl)-1H-benzo[d]imidazol-6-yl)(cyclopropyl)methyl)-2-(3,3-difluorocyclobutyl)acetamide hydrochloride (Intermediate 371) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 4-methyl-1,2,5-oxadiazole-3-carboxylic acid in place of 1-isopropyl-1H-pyrazole-5-carboxylic acid. The crude product was purified by silica gel chromatography (0-10% MeOH/DCM) to afford the title compounds as a mixture of diastereomers, which were separated by SFC using a chiral stationary phase (DAICEL CHIRALPAK IG, 10 μm, 250×30 mm, 75% CO₂ in IPA (0.1% NH₄OH)). The first eluting fraction was Example 244 and the second eluting fraction was Example 245. Example 244 required additional purification by basic preparative HPLC (Phenomenex Gemini-NX, 5 μm, C18, 150×30 mm, 40-70% MeCN/water (with 0.05% NH₄OH)). The product containing fractions were diluted with water, frozen, and lyophilized to afford the title compounds as white solids. Example 244: H NMR (400 MHz, DMSO-d₆) δ 12.20 (s, 1H), 9.52 (d, J=7.6 Hz, 1H), 8.52-8.42 (m, 1H), 7.57-7.44 (m, 1H), 7.44-7.35 (m, 1H), 7.14 (d, J=8.4 Hz, 1H), 5.21 (t, J=8.4 Hz, 1H), 4.34 (t, J=8.4 Hz, 1H), 3.04-2.82 (m, 1H), 2.69-2.53 (m, 2H), 2.50 (s, 3H), 2.42-2.23 (m, 9H), 2.21-2.08 (m, 1H), 1.82-1.69 (m, 2H), 1.21-1.11 (m, 1H), 0.54-0.42 (m, 2H), 0.36-0.26 (m, 2H). MS (ESI) m/z: [M+H]⁺ Found 595.3. Example 245: ¹H NMR (400 MHz, DMSO-d₆) δ 12.28 (s, 1H), 9.62-9.50 (m, 1H), 8.54-8.42 (m, 1H), 7.56-7.48 (m, 1H), 7.43-7.38 (m, 1H), 7.19-7.11 (m, 1H), 5.41-5.32 (m, 1H), 4.38-4.29 (m, 1H), 3.07-2.96 (m, 1H), 2.68-2.55 (m, 3H), 2.49 (s, 3H), 2.46-2.23 (m, 7H), 2.17-2.05 (m, 2H), 2.03-1.87 (m, 2H), 1.21-1.11 (m, 1H), 0.54-0.42 (m, 2H), 0.35-0.27 (m, 2H). MS (ESI) m/z: [M+H]⁺ Found 595.1.

### Example 246

### N—((S)-(6-((R)-Cyclopropyl(2-(3,3-difluorocyclobutyl)acetamido)methyl)-1H-benzo[d]imidazol-2-yl)((R)-3,3-difluorocyclohexyl)methyl)-4-methyl-1,2,5-oxadiazole-3-carboxamide

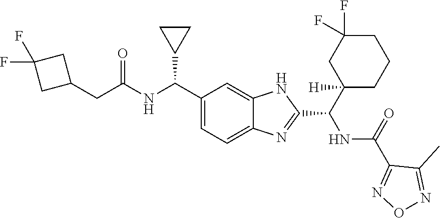 [SMILES: Cc1nonc1C(=O)N[C@H](c1nc2ccc([C@H](NC(=O)CC3CC(F)(F)C3)C3CC3)cc2[nH]1)[C@@H]1CCCC(F)(F)C1]

The title compound was prepared as described for the synthesis of Example 233, using N—((R)-(2-((S)-amino((S)-3,3-difluorocyclohexyl)methyl)-1H-benzo[d]imidazol-6-yl)(cyclopropyl)methyl)-2-(3,3-difluorocyclobutyl)acetamide (Intermediate 375) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (50-100% EtOAc/petroleum ether) followed by SFC using a chiral stationary phase twice (DAICEL CHIRALCEL OD-H, 5 μm, 250×30 mm, 80% CO₂ in EtOH (0.1% NH₄OH)). The product containing fractions were diluted with water, frozen, and lyophilized to afford the title compound as a white solid. ¹H NMR (400 MHz, DMSO-d₆) δ 12.34 (s, 1H), 9.55 (s, 1H), 8.57-8.37 (m, 1H), 7.65-7.49 (m, 1H), 7.47-7.35 (m, 1H), 7.18 (s, 1H), 5.23-5.20 (m, 1H), 4.36-4.32 (m, 1H), 2.71-2.52 (m, 3H), 2.47 (s, 3H), 2.46-2.25 (m, 5H), 2.02-1.58 (m, 6H), 1.41-1.38 (m, 1H), 1.22-1.17 (m, 2H), 0.51-0.48 (m, 2H), 0.33-0.31 (m, 2H). MS (ESI) m/z: [M+H]⁺ Found 577.3.

### Example 247

### N—((S)-(6-((R)-Cyclopropyl(2-(3,3-difluorocyclobutyl)acetamido)methyl)-1H-benzo[d]imidazol-2-yl)((S)-3,3-difluorocyclohexyl)methyl)-4-methyl-1,2,5-oxadiazole-3-carboxamide

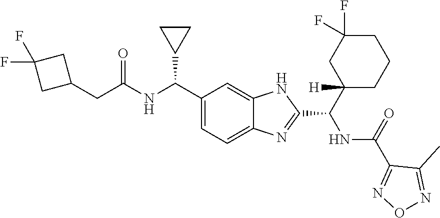 [SMILES: Cc1nonc1C(=O)N[C@H](c1nc2ccc([C@H](NC(=O)CC3CC(F)(F)C3)C3CC3)cc2[nH]1)[C@H]1CCCC(F)(F)C1]

The title compound was prepared as described for the synthesis of Example 233, using N—((R)-(2-((S)-amino((R)-3,3-difluorocyclohexyl)methyl)-1H-benzo[d]imidazol-6-yl)(cyclopropyl)methyl)-2-(3,3-difluorocyclobutyl)acetamide (Intermediate 373) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (60-80% EtOAc/petroleum ether) followed by SFC using a chiral stationary phase twice (DAICEL CHIRALCEL OJ-H, 5 μm, 250×30 mm, 85% CO₂ in EtOH (0.1% NH₄OH) followed by DAICEL CHIRALPAK AD, 10 μm, 250×30 mm, 70% CO₂ in EtOH (0.1% NH₄OH)). The product containing fractions were diluted with water, frozen, and lyophilized to afford the title compound as a white solid. ¹H NMR (400 MHz, DMSO-d₆) δ 12.33 (s, 1H), 9.69-9.46 (m, 1H), 8.52-8.46 (m, 1H), 7.59-7.52 (m, 1H), 7.46-7.39 (m, 1H), 7.18-7.16 (m, 1H), 5.26-5.22 (m, 1H), 4.37-4.31 (m, 1H), 2.63-2.62 (m, 2H), 2.49 (s, 3H), 2.46-2.16 (m, 7H), 2.01-1.94 (m, 1H), 1.83-1.72 (m, 2H), 1.71-1.62 (m, 1H), 1.55-1.52 (m, 1H), 1.47-1.31 (m, 1H), 1.26-1.05 (m, 2H), 0.51-0.47 (m, 2H), 0.36-0.29 (m, 2H). MS (ESI) m/z: [M+H]⁺ Found 577.1.

### Example 248

### N—((R*)-(6-((R)-Cyclopropyl(2-(3,3-difluorocyclobutyl)acetamido)methyl)-1H-benzo[d]imidazol-2-yl)((S*)-tetrahydro-2H-pyran-2-yl)methyl)-4-methyl-1,2,5-oxadiazole-3-carboxamide

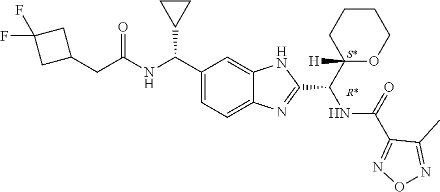 [SMILES: Cc1nonc1C(=O)N[C@H](c1nc2ccc([C@H](NC(=O)CC3CC(F)(F)C3)C3CC3)cc2[nH]1)[C@@H]1CCCCO1]

### Example 249

### N—((S*)-(6-((R)-Cyclopropyl(2-(3,3-difluorocyclobutyl)acetamido)methyl)-1H-benzo[d]imidazol-2-yl)((S*)-tetrahydro-2H-pyran-2-yl)methyl)-4-methyl-1,2,5-oxadiazole-3-carboxamide

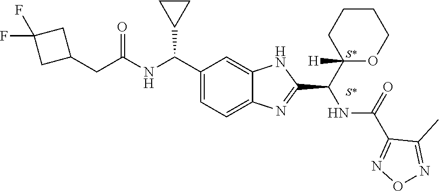 [SMILES: Cc1nonc1C(=O)N[C@@H](c1nc2ccc([C@H](NC(=O)CC3CC(F)(F)C3)C3CC3)cc2[nH]1)[C@@H]1CCCCO1]

The title compounds were prepared as described for the synthesis of Example 234, using N—((R)-(2-((R)-amino((S*)-tetrahydro-2H-pyran-2-yl)methyl)-1H-benzo[d]imidazol-6-yl)(cyclopropyl)methyl)-2-(3,3-difluorocyclobutyl)acetamide hydrochloride (Intermediate 377) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 4-methyl-1,2,5-oxadiazole-3-carboxylic acid in place of 1-isopropyl-1H-pyrazole-5-carboxylic acid. The crude product was purified by silica gel chromatography (0-10% MeOH/DCM) to afford the title compounds as a mixture of diastereomers that were separated by SFC using a chiral stationary phase (Phenomenex-Cellulose-2, 10 μm, 250×30 mm, 75% CO₂ in MeOH (0.1% NH₄OH)). The first eluting fraction was Example 248 and the second eluting fraction was Example 249. The product containing fractions were diluted with water, frozen, and lyophilized to afford the title compounds as white solids. Example 248: ¹H NMR (400 MHz, DMSO-d₆) δ 12.33 (s, 1H), 9.25-9.20 (m, 1H), 8.51-8.44 (m, 1H), 7.56-7.49 (m, 1H), 7.43-7.39 (m, 1H), 7.18-7.13 (m, 1H), 5.27-5.22 (m, 1H), 4.36-4.29 (m, 1H), 3.98-3.88 (m, 2H), 2.68-2.56 (m, 2H), 2.49-2.48 (m, 3H), 2.41-2.23 (m, 5H), 1.81-1.71 (m, 1H), 1.50-1.37 (m, 4H), 1.37-1.21 (m, 2H), 1.20-1.12 (m, 1H), 0.53-0.42 (m, 2H), 0.35-0.28 (m, 2H). MS (ESI) m/z: [M+H]⁺ Found 543.4. Example 249: ¹H NMR (400 MHz, DMSO-d₆) δ 12.28 (s, 1H), 9.43 (d, J=8.8 Hz, 1H), 8.48 (d, J=7.6 Hz, 1H), 7.58-7.35 (m, 2H), 7.15 (d, J=7.6 Hz, 1H), 5.26-5.20 (m, 1H), 4.32 (t, J=8.4 Hz, 1H), 3.96-3.81 (m, 2H), 2.68-2.57 (m, 2H), 2.48-2.45 (m, 3H), 2.41-2.20 (m, 5H), 1.85-1.75 (m, 2H), 1.50-1.22 (m, 5H), 1.20-1.12 (m, 1H), 0.53-0.43 (m, 2H), 0.35-0.28 (m, 2H). MS (ESI) m/z: [M+H]⁺ Found 543.3.

### Example 250

### N—((R*)-(6-((R)-Cyclopropyl(2-(3,3-difluorocyclobutyl)acetamido)methyl)-1H-benzo[d]imidazol-2-yl)((S*)-tetrahydro-2H-pyran-2-yl)methyl)-1-isopropyl-1H-pyrazole-5-carboxamide

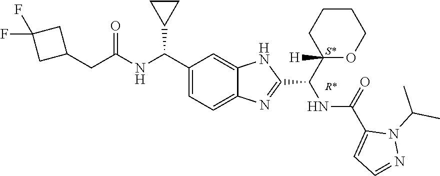 [SMILES: CC(C)n1nccc1C(=O)N[C@H](c1nc2ccc([C@H](NC(=O)CC3CC(F)(F)C3)C3CC3)cc2[nH]1)[C@@H]1CCCCO1]

### Example 251

### N—((S*)-(6-((R)-Cyclopropyl(2-(3,3-difluorocyclobutyl)acetamido)methyl)-1H-benzo[d]imidazol-2-yl)((S*)-tetrahydro-2H-pyran-2-yl)methyl)-1-isopropyl-1H-pyrazole-5-carboxamide

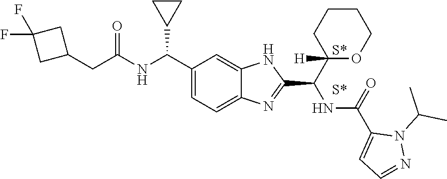 [SMILES: CC(C)n1nccc1C(=O)N[C@@H](c1nc2ccc([C@H](NC(=O)CC3CC(F)(F)C3)C3CC3)cc2[nH]1)[C@@H]1CCCCO1]

The title compounds were prepared as described for the synthesis of Example 233, using N—((R)-(2-((R)-amino((S*)-tetrahydro-2H-pyran-2-yl)methyl)-1H-benzo[d]imidazol-6-yl)(cyclopropyl)methyl)-2-(3,3-difluorocyclobutyl)acetamide hydrochloride (Intermediate 377) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 1-isopropyl-1H-pyrazole-5-carboxylic acid in place of 4-methyl-1,2,5-oxadiazole-3-carboxylic acid. The crude product was purified by silica gel chromatography (0-2% MeOH/DCM) to afford the title compounds as a mixture of diastereomers which were separated by SFC using a chiral stationary phase (DAICEL CHIRALPAK AD-H, 5 μm, 250×30 mm, 70% CO₂ in IPA (0.1% NH₄OH)). The first eluting fraction was Example 251 and the second eluting fraction was Example 250. The product containing fractions were diluted with water, frozen, and lyophilized to afford the title compounds as white solids. Example 250: ¹H NMR (400 MHz, DMSO-d₆) δ 12.26 (s, 1H), 8.83-8.76 (m, 1H), 8.53-8.44 (m, 1H), 7.55-7.46 (m, 2H), 7.41-7.36 (m, 1H), 7.18-7.11 (m, 1H), 6.97-6.94 (m, 1H), 5.44-5.35 (m, 1H), 5.19 (t, J=7.6 Hz, 1H), 4.32 (q, J=8.0 Hz, 1H), 3.95-3.77 (m, 2H), 3.33-3.27 (m, 1H), 2.69-2.57 (m, 2H), 2.41-2.21 (m, 5H), 1.86-1.75 (m, 2H), 1.52-1.41 (m, 3H), 1.36 (d, J=6.8 Hz, 3H), 1.32 (d, J=6.4 Hz, 3H), 1.29-1.22 (m, 1H), 1.20-1.11 (m, 1H), 0.54-0.42 (m, 2H), 0.35-0.26 (m, 2H). MS (ESI) m/z: [M+H]⁺ Found 569.2. Example 251: ¹H NMR (400 MHz, DMSO-d₆) δ 12.32 (s, 1H), 8.67 (d, J=8.4 Hz, 1H), 8.49 (d, J=8.4 Hz, 1H), 7.56-7.43 (m, 3H), 7.16 (d, J=8.4 Hz, 1H), 7.00 (d, J=2.0 Hz, 1H), 5.46-5.36 (m, 1H), 5.29-5.22 (m, 1H), 4.33 (t, J=8.4 Hz, 1H), 3.96-3.86 (m, 2H), 3.36-3.33 (m, 1H), 2.71-2.56 (m, 2H), 2.43-2.20 (m, 5H), 1.80-1.70 (m, 1H), 1.49-1.40 (m, 3H), 1.37 (d, J=6.4 Hz, 3H), 1.34 (d, J=6.4 Hz, 3H), 1.31-1.22 (m, 2H), 1.20-1.12 (m, 1H), 0.54-0.43 (m, 2H), 0.36-0.27 (m, 2H). MS (ESI) m/z: [M+H]⁺ Found 569.2.

### Example 252

### N—((R)-(6-((R)-Cyclopropyl(2-(3,3-difluorocyclobutyl)acetamido)methyl)-1H-benzo[d]imidazol-2-yl)((R*)-tetrahydro-2H-pyran-2-yl)methyl)-4-methyl-1,2,5-oxadiazole-3-carboxamide

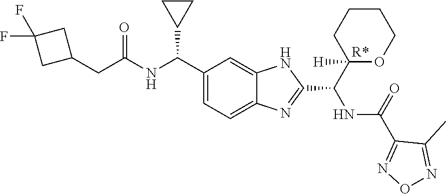 [SMILES: Cc1nonc1C(=O)N[C@H](c1nc2ccc([C@H](NC(=O)CC3CC(F)(F)C3)C3CC3)cc2[nH]1)[C@H]1CCCCO1]

The title compound was prepared as described for the synthesis of Example 233, using N—((R)-(2-((R)-amino((R*)-tetrahydro-2H-pyran-2-yl)methyl)-1H-benzo[d]imidazol-6-yl)(cyclopropyl)methyl)-2-(3,3-difluorocyclobutyl)acetamide hydrochloride (Intermediate 379) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (0-65% EtOAc/petroleum ether) followed by SFC using a chiral stationary phase (DAICEL CHIRALCEL OD-H, 5 μm, 250×30 mm, 80% CO₂ in EtOH (0.1% NH₄OH)). The product containing fractions were diluted with water, frozen, and lyophilized to afford the title compound as a white solid. ¹H NMR (400 MHz, DMSO-d₆) δ 12.27 (s, 1H), 9.43 (dd, J=4.4, 8.8 Hz, 1H), 8.48 (dd, J=8.4, 13.2 Hz, 1H), 7.56-7.47 (m, 1H), 7.42-7.35 (m, 1H), 7.20-7.07 (m, 1H), 5.29-5.18 (m, 1H), 4.32 (q, J=8.4 Hz, 1H), 3.93-3.79 (m, 2H), 2.73-2.57 (m, 3H), 2.47 (s, 3H), 2.42-2.21 (m, 5H), 1.80 (d, J=10.0 Hz, 2H), 1.55-1.38 (m, 3H), 1.36-1.09 (m, 2H), 0.57-0.41 (m, 2H), 0.34-0.28 (m, 2H). MS (ESI) m/z: [M+H]⁺ Found 543.3.

### Example 253

### N—((R)-(6-((R)-Cyclopropyl(2-(3,3-difluorocyclobutyl)acetamido)methyl)-1H-benzo[d]imidazol-2-yl)((R*)-tetrahydro-2H-pyran-2-yl)methyl)-1-isopropyl-1H-pyrazole-5-carboxamide

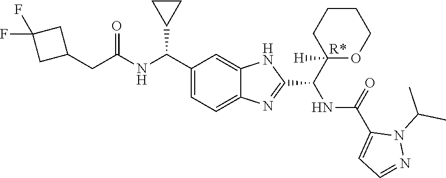 [SMILES: CC(C)n1nccc1C(=O)N[C@H](c1nc2ccc([C@H](NC(=O)CC3CC(F)(F)C3)C3CC3)cc2[nH]1)[C@H]1CCCCO1]

The title compound was prepared as described for the synthesis of Example 234, using N—((R)-(2-((R)-amino((R*)-tetrahydro-2H-pyran-2-yl)methyl)-1H-benzo[d]imidazol-6-yl)(cyclopropyl)methyl)-2-(3,3-difluorocyclobutyl)acetamide hydrochloride (Intermediate 379) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (0-5% MeOH/DCM) to afford the title compound. The product containing fractions were concentrated to dryness under reduced pressure, diluted with water, frozen, and lyophilized to afford the title compound as a white solid. ¹H NMR (400 MHz, DMSO-d₆) δ 12.24 (s, 1H), 8.80-8.78 (m, 1H), 8.56-8.32 (m, 1H), 7.56-7.45 (m, 2H), 7.42-7.32 (m, 1H), 7.14-7.12 (m, 1H), 6.96 (d, J=2.0 Hz, 1H), 5.43-5.36 (m, 1H), 5.19 (t, J=8.4 Hz, 1H), 4.32 (t, J=8.4 Hz, 1H), 3.96-3.76 (m, 2H), 2.70-2.57 (m, 2H), 2.42-2.16 (m, 5H), 1.89-1.73 (m, 2H), 1.52-1.50 (m, 3H), 1.36-1.30 (m, 7H), 1.25-1.08 (m, 2H), 0.49-0.45 (m, 2H), 0.33-0.30 (m, 2H). MS (ESI) m/z: [M+H]⁺ Found 569.3.

### Example 254

### N—((S)-((1R,3s,5S)-Bicyclo[3.1.0]hexan-3-yl)(6-((R)-cyclopropyl(2-(3,3-difluorocyclobutyl)acetamido)methyl)-1H-benzo[d]imidazol-2-yl)methyl)-4-methyl-1,2,5-oxadiazole-3-carboxamide

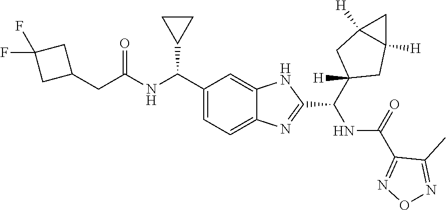 [SMILES: Cc1nonc1C(=O)N[C@H](c1nc2ccc([C@H](NC(=O)CC3CC(F)(F)C3)C3CC3)cc2[nH]1)[C@@H]1C[C@H]2C[C@H]2C1]

The title compound was prepared as described for the synthesis of Example 233, using N—((R)-(2-((S)-amino((1R,3s,5S)-bicyclo[3.1.0]hexan-3-yl)methyl)-1H-benzo[d]imidazol-6-yl)(cyclopropyl)methyl)-2-(3,3-difluorocyclobutyl)acetamide (Intermediate 383) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (0-5% MeOH/DCM) followed by preparative HPLC (Phenomenex Gemini-NX, 3 μm, C18, 75×30 mm, 40-70% MeCN/water (with 0.05% NH₄OH), and SFC using a chiral stationary phase (DAICEL CHIRALPAK AD, 10 μm, 250×30 mm, 75% CO₂ in EtOH (0.1% NH₄OH)). The product containing fractions were diluted with water, frozen, and lyophilized to afford the title compound as a white solid. ¹H NMR (400 MHz, DMSO-d₆) δ 12.28 (s, 1H), 9.53 (dd, J=4.8, 8.4 Hz, 1H), 8.50 (dd, J=8.8, 12.0 Hz, 1H), 7.56-7.47 (m, 1H), 7.43-7.36 (m, 1H), 7.19-7.12 (m, 1H), 5.09 (t, J=8.8 Hz, 1H), 4.32 (dt, J=3.2, 8.4 Hz, 1H), 2.47 (s, 3H), 2.42-2.23 (m, 6H), 1.95 (dd, J=7.2, 12.3 Hz, 1H), 1.68-1.62 (m, 1H), 1.68-1.62 (m, 1H), 1.57 (d, J=8.8 Hz, 2H), 1.24 (d, J=6.4 Hz, 3H), 1.20-1.11 (m, 1H), 0.55-0.41 (m, 2H), 0.36-0.16 (m, 4H). MS (ESI) m/z: [M+H]⁺ Found 539.2.

### Example 255

### N—((S)-((1R,3s,5S)-Bicyclo[3.1.0]hexan-3-yl)(6-((R)-cyclopropyl(2-(3,3-difluorocyclobutyl)acetamido)methyl)-1H-benzo[d]imidazol-2-yl)methyl)-1-isopropyl-1H-pyrazole-5-carboxamide

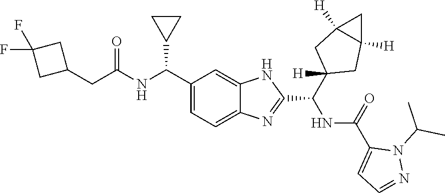 [SMILES: CC(C)n1nccc1C(=O)N[C@H](c1nc2ccc([C@H](NC(=O)CC3CC(F)(F)C3)C3CC3)cc2[nH]1)[C@@H]1C[C@H]2C[C@H]2C1]

The title compound was prepared as described for the synthesis of Example 234, using N—((R)-(2-((S)-amino((1R,3s,5S)-bicyclo[3.1.0]hexan-3-yl)methyl)-1H-benzo[d]imidazol-6-yl)(cyclopropyl)methyl)-2-(3,3-difluorocyclobutyl)acetamide (Intermediate 383) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (0-5% MeOH/DCM) followed by preparative TLC (5% MeOH/DCM). The product containing fractions were concentrated then the product was diluted with water, frozen, and lyophilized to afford the title compound. ¹H NMR (400 MHz, DMSO-d₆) δ 12.24 (s, 1H), 8.81 (d, J=8.4 Hz, 1H), 8.53-8.44 (m, 1H), 7.55-7.37 (m, 3H), 7.18-7.11 (m, 1H), 6.94 (d, J=2.0 Hz, 1H), 5.45-5.35 (m, 1H), 5.10-5.02 (m, 1H), 4.36-4.31 (m, 1H), 2.72-2.56 (m, 2H), 2.44-2.21 (m, 6H), 2.02-1.97 (m, 1H), 1.68-1.50 (m, 3H), 1.39-1.30 (m, 6H), 1.28-1.20 (m, 2H), 1.20-1.11 (m, 1H), 0.54-0.43 (m, 2H), 0.36-0.25 (m, 3H), 0.21-0.16 (m, 1H). MS (ESI) m/z: [M+H]⁺ Found 565.5.

### Example 256

### N—((R)-1-(6-((R)-Cyclopropyl(2-(3,3-difluorocyclobutyl)acetamido)methyl)-1H-benzo[d]imidazol-2-yl)-2-(3,3-difluorocyclobutoxy)ethyl)-4-methyl-1,2,5-oxadiazole-3-carboxamide

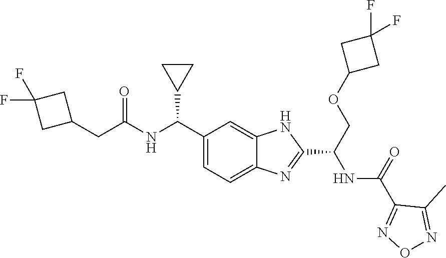 [SMILES: Cc1nonc1C(=O)N[C@@H](COC1CC(F)(F)C1)c1nc2ccc([C@H](NC(=O)CC3CC(F)(F)C3)C3CC3)cc2[nH]1]

The title compound was prepared as described for the synthesis of Example 234, using N—((R)-(2-((R)-1-amino-2-(3,3-difluorocyclobutoxy)ethyl)-1H-benzo[d]imidazol-6-yl)(cyclopropyl)methyl)-2-(3,3-difluorocyclobutyl)acetamide (Intermediate 385) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 4-methyl-1,2,5-oxadiazole-3-carboxylic acid in place of 1-isopropyl-1H-pyrazole-5-carboxylic acid. The crude product was purified by preparative TLC (5% MeOH/DCM) followed by SFC using a chiral stationary phase (Phenomenex-Cellulose-2, 5 μm, 250×30 mm, 80% CO₂ in MeOH (0.1% NH₄OH)). The product containing fractions were diluted with water, frozen, and lyophilized to afford the title compound as a white solid. ¹H NMR (400 MHz, DMSO-d₆) δ 12.34 (s, 1H), 9.69-9.63 (m, 1H), 8.54-8.44 (m, 1H), 7.57-7.48 (m, 1H), 7.42-7.37 (m, 1H), 7.20-7.12 (m, 1H), 5.51-5.43 (m, 1H), 4.38-4.30 (m, 1H), 4.17-4.00 (m, 2H), 3.95-3.87 (m, 1H), 2.96-2.82 (m, 2H), 2.71-2.54 (m, 4H), 2.37-2.33 (m, 3H), 2.32-1.96 (m, 2H), 1.36-1.05 (m, 4H), 0.54-0.41 (m, 2H), 0.35-0.26 (m, 2H). MS (ESI) m/z: [M+H]⁺ Found 579.3.

### Example 257

### N—((R)-1-(6-((R)-Cyclopropyl(2-(3,3-difluorocyclobutyl)acetamido)methyl)-1H-benzo[d]imidazol-2-yl)-2-(3,3-difluorocyclobutoxy)ethyl)-1-isopropyl-1H-pyrazole-5-carboxamide

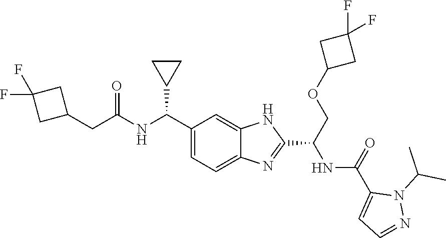 [SMILES: CC(C)n1nccc1C(=O)N[C@@H](COC1CC(F)(F)C1)c1nc2ccc([C@H](NC(=O)CC3CC(F)(F)C3)C3CC3)cc2[nH]1]

The title compound was prepared as described for the synthesis of Example 233, using N—((R)-(2-((R)-1-amino-2-(3,3-difluorocyclobutoxy)ethyl)-1H-benzo[d]imidazol-6-yl)(cyclopropyl)methyl)-2-(3,3-difluorocyclobutyl)acetamide (Intermediate 385) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 1-isopropyl-1H-pyrazole-5-carboxylic acid in place of 4-methyl-1,2,5-oxadiazole-3-carboxylic acid. The crude product was purified by silica gel chromatography (5-10% MeOH/DCM) followed by SFC using a chiral stationary phase (DAICEL CHIRALCEL OD-H, 5 μm, 250×30 mm, 80% CO₂ in EtOH (0.1% NH₄OH)). The product containing fractions were diluted with water, frozen, and lyophilized to afford the title compound as a white solid. ¹H NMR (400 MHz, DMSO-d₆) δ 12.33 (s, 1H), 8.96-8.93 (m, 1H), 8.51-8.45 (m, 1H), 7.58-7.46 (m, 2H), 7.43-7.35 (m, 1H), 7.17-7.14 (m, 1H), 6.94 (d, J=2.0 Hz, 1H), 5.53-5.39 (m, 2H), 4.37-4.30 (m, 1H), 4.18-4.06 (m, 1H), 4.03-3.98 (m, 1H), 3.92-3.76 (m, 1H), 2.95-2.80 (m, 2H), 2.71-2.54 (m, 4H), 2.42-2.18 (m, 5H), 1.38 (s, 3H), 1.36 (s, 3H), 1.19-1.09 (m, 1H), 0.54-0.39 (m, 2H), 0.33-0.27 (m, 2H). MS (ESI) m/z: [M+H]⁺ Found 605.3.

### Example 258

### N—((S)-(5-((R)-Cyclopropyl((R*)-4,4,4-trifluoro-3-methylbutanamido)methyl)-1H-benzo[d]imidazol-2-yl)((R)-3,3-difluorocyclohexyl)methyl)-4-methyl-1,2,5-oxadiazole-3-carboxamide

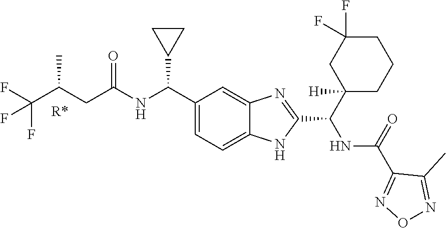 [SMILES: Cc1nonc1C(=O)N[C@H](c1nc2cc([C@H](NC(=O)C[C@@H](C)C(F)(F)F)C3CC3)ccc2[nH]1)[C@@H]1CCCC(F)(F)C1]

### Example 259

### N—((S)-(5-((R)-Cyclopropyl((S*)-4,4,4-trifluoro-3-methylbutanamido)methyl)-1H-benzo[d]imidazol-2-yl)((R)-3,3-difluorocyclohexyl)methyl)-4-methyl-1,2,5-oxadiazole-3-carboxamide

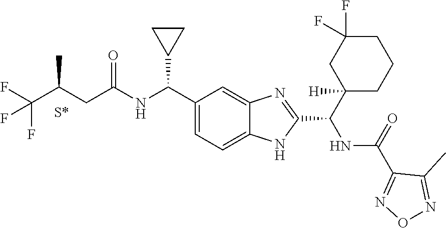 [SMILES: Cc1nonc1C(=O)N[C@H](c1nc2cc([C@H](NC(=O)C[C@H](C)C(F)(F)F)C3CC3)ccc2[nH]1)[C@@H]1CCCC(F)(F)C1]

The title compounds were prepared as described for the synthesis of Example 233, using N—((R)-(2-((S)-amino((R)-3,3-difluorocyclohexyl)methyl)-1H-benzo[d]imidazol-5-yl)(cyclopropyl)methyl)-4,4,4-trifluoro-3-methylbutanamide hydrochloride (Intermediate 381) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (0-5% MeOH/DCM) and preparative TLC (5% MeOH/DCM) to afford the title compounds as a mixture of diastereomers. The diastereomers were separated by SFC using a chiral stationary phase (DAICEL CHIRALCEL IC, 5 μm, 150×20 mm, 90% CO₂ in IPA). The first eluting fraction was Example 258 and the second eluting fraction was Example 259. The product containing fractions were diluted with water, frozen, and lyophilized to afford the title compounds as white solids. Example 258: ¹H NMR (600 MHz, DMSO-d₆) δ 12.33 (s, 1H), 9.53 (d, J=8.5 Hz, 1H), 8.62 (s, 1H), 7.64-7.51 (m, 1H), 7.49-7.39 (m, 1H), 7.19 (d, J=8.2 Hz, 1H), 5.22 (t, J=8.1 Hz, 1H), 4.37 (t, J=8.6 Hz, 1H), 2.86-2.66 (m, 1H), 2.49-2.46 (m, 3H), 2.46-2.41 (m, 1H), 2.21 (dd, J=14.5, 9.4 Hz, 1H), 2.04-1.95 (m, 1H), 1.95-1.78 (m, 3H), 1.78-1.62 (m, 2H), 1.47-1.35 (m, 1H), 1.26-1.14 (m, 3H), 0.98 (d, J=6.9 Hz, 3H), 0.55-0.42 (m, 2H), 0.36-0.31 (m, 2H). MS (ESI) m/z: [M+H]⁺ Found 583.1. Example 259: ¹H NMR (600 MHz, DMSO-d₆) δ 12.34 (s, 1H), 9.53 (d, J=8.5 Hz, 1H), 8.62 (s, 1H), 7.69-7.53 (m, 1H), 7.51-7.41 (m, 1H), 7.20 (d, J=8.4 Hz, 1H), 5.23 (t, J=8.1 Hz, 1H), 4.40 (t, J=8.5 Hz, 1H), 2.86-2.71 (m, 1H), 2.49 (s, 3H), 2.45 (dd, J=14.5, 4.5 Hz, 1H), 2.25 (dd, J=14.5, 9.6 Hz, 1H), 2.03-1.95 (m, 1H), 1.95-1.79 (m, 3H), 1.79-1.62 (m, 2H), 1.47-1.37 (m, 1H), 1.27-1.15 (m, 3H), 1.08 (d, J=6.9 Hz, 3H), 0.56-0.45 (m, 2H), 0.40-0.30 (m, 2H). MS (ESI) m/z: [M+H]+ Found 583.1

### Example 260

### N—((R)-(6-((R)-Cyclopropyl(2-(3,3-difluorocyclobutyl)acetamido)methyl)-1H-benzo[d]imidazol-2-yl)((S*)-5,5-difluorotetrahydro-2H-pyran-2-yl)methyl)-4-methyl-1,2,5-oxadiazole-3-carboxamide

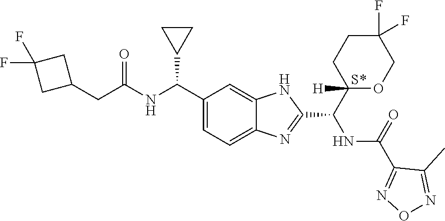 [SMILES: Cc1nonc1C(=O)N[C@H](c1nc2ccc([C@H](NC(=O)CC3CC(F)(F)C3)C3CC3)cc2[nH]1)[C@@H]1CCC(F)(F)CO1]

The title compound was prepared as described for the synthesis of Example 233, using N—((R)-(2-((R)-amino((S*)-5,5-difluorotetrahydro-2H-pyran-2-yl)methyl)-1H-benzo[d]imidazol-6-yl)(cyclopropyl)methyl)-2-(3,3-difluorocyclobutyl)acetamide hydrochloride (Intermediate 386) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide. The crude product was purified by silica gel chromatography (0-2% MeOH/DCM) followed by SFC using a chiral stationary phase (DAICEL CHIRALCEL OD-H, 5 μm, 250×30 mm, 75% CO₂ in EtOH (0.1% NH₄OH)). The product containing fractions were diluted with water, frozen, and lyophilized to afford the title compound as a white solid. ¹H NMR (400 MHz, DMSO-d₆) δ 12.39 (s, 1H), 9.60 (dd, J=5.2, 8.4 Hz, 1H), 8.51 (dd, J=8.8, 12.2 Hz, 1H), 7.58-7.50 (m, 1H), 7.43-7.39 (m, 1H), 7.19-7.14 (m, 1H), 5.38-5.30 (m, 1H), 4.36-4.28 (m, 1H), 4.19-4.11 (m, 1H), 3.94-3.83 (m, 1H), 3.73-3.56 (m, 1H), 2.70-2.56 (m, 2H), 2.48 (s, 3H), 2.41-2.15 (m, 6H), 2.13-1.96 (m, 2H), 1.75-1.58 (m, 1H), 1.22-1.11 (m, 1H), 0.56-0.41 (m, 2H), 0.36-0.26 (m, 2H). MS (ESI) m/z: [M+H]+ Found 579.1.

### Example 261

### N—((R)-(6-((R)-Cyclopropyl(2-(3,3-difluorocyclobutyl)acetamido)methyl)-1H-benzo[d]imidazol-2-yl)((S*)-5,5-difluorotetrahydro-2H-pyran-2-yl)methyl)-1-isopropyl-1H-pyrazole-5-carboxamide

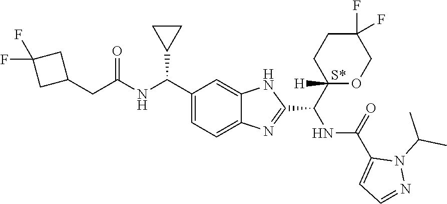 [SMILES: CC(C)n1nccc1C(=O)N[C@H](c1nc2ccc([C@H](NC(=O)CC3CC(F)(F)C3)C3CC3)cc2[nH]1)[C@@H]1CCC(F)(F)CO1]

The title compound was prepared as described for the synthesis of Example 233, using N—((R)-(2-((R)-amino((S*)-5,5-difluorotetrahydro-2H-pyran-2-yl)methyl)-1H-benzo[d]imidazol-6-yl)(cyclopropyl)methyl)-2-(3,3-difluorocyclobutyl)acetamide hydrochloride (Intermediate 386) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 1-isopropyl-1H-pyrazole-5-carboxylic acid in place of 4-methyl-1,2,5-oxadiazole-3-carboxylic acid. The crude product was purified by silica gel chromatography (0-2% MeOH/DCM) followed by SFC using a chiral stationary phase (DAICEL CHIRALPAK IG, 10 μm, 250×30 mm, 70% CO₂ in EtOH (0.1% NH₄OH)). The product containing fractions were diluted with water, frozen, and lyophilized to afford the title compound as a white solid. ¹H NMR (400 MHz, DMSO-d₆) δ 12.37 (s, 1H), 8.94 (dd, J=4.4, 8.8 Hz, 1H), 8.49 (s, 1H), 7.56-7.49 (m, 2H), 7.42-7.38 (m, 1H), 7.19-7.13 (m, 1H), 6.96 (d, J=1.6 Hz, 1H), 5.44-5.34 (m, 1H), 5.29 (t, J=8.0 Hz, 1H), 4.37-4.28 (m, 1H), 4.16-4.08 (m, 1H), 3.90-3.81 (m, 1H), 3.68-3.56 (m, 1H), 2.71-2.57 (m, 2H), 2.42-2.16 (m, 6H), 2.11-1.91 (m, 2H), 1.72-1.58 (m, 1H), 1.41-1.28 (m, 6H), 1.22-1.11 (m, 1H), 0.55-0.40 (m, 2H), 0.35-0.24 (m, 2H). MS (ESI) m/z: [M+H]⁺ Found 605.2.

### Example 262

### N—((R)-(6-((R)-Cyclopropyl(2-(3,3-difluorocyclobutyl)acetamido)methyl)-1H-benzo[d]imidazol-2-yl)((R*)-5,5-difluorotetrahydro-2H-pyran-2-yl)methyl)-1-isopropyl-1H-pyrazole-5-carboxamide

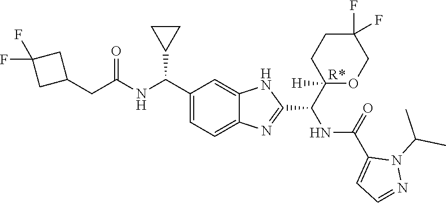 [SMILES: CC(C)n1nccc1C(=O)N[C@H](c1nc2ccc([C@H](NC(=O)CC3CC(F)(F)C3)C3CC3)cc2[nH]1)[C@H]1CCC(F)(F)CO1]

The title compound was prepared as described for the synthesis of Example 233, using N—((R)-(2-((R)-amino((R*)-5,5-difluorotetrahydro-2H-pyran-2-yl)methyl)-1H-benzo[d]imidazol-6-yl)(cyclopropyl)methyl)-2-(3,3-difluorocyclobutyl)acetamide hydrochloride (Intermediate 387) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 1-isopropyl-1H-pyrazole-5-carboxylic acid in place of 4-methyl-1,2,5-oxadiazole-3-carboxylic acid. The crude product was purified by silica gel chromatography (0-2% MeOH/DCM) followed by SFC using a chiral stationary phase (DAICEL CHIRALPAK IC, 10 μm, 250×30 mm, 80% CO₂ in EtOH (0.1% NH₄OH)). The product containing fractions were diluted with water, frozen, and lyophilized to afford the title compound as a white solid. ¹H NMR (400 MHz, DMSO-d₆) δ 12.39 (s, 1H), 8.88 (d, J=8.4 Hz, 1H), 8.50 (d, J=7.6 Hz, 1H), 7.59-7.36 (m, 3H), 7.17 (d, J=8.4 Hz, 1H), 6.98 (d, J=2.0 Hz, 1H), 5.46-5.31 (m, 2H), 4.33 (t, J=8.4 Hz, 1H), 4.18-4.11 (m, 1H), 3.99-3.90 (m, 1H), 3.70-3.56 (m, 1H), 2.71-2.55 (m, 2H), 2.44-2.21 (m, 5H), 2.19-1.95 (m, 2H), 1.73-1.53 (m, 2H), 1.39-1.31 (m, 6H), 1.22-1.10 (m, 1H), 0.54-0.42 (m, 2H), 0.35-0.27 (m, 2H). MS (ESI) m/z: [M+H]⁺ Found 605.1.

### Example 263

### N—((S)-(5-((R)-Cyclopropyl(4,4,4-trifluorobutanamido)methyl)-1H-benzo[d]imidazol-2-yl)((R)-3,3-difluorocyclohexyl)methyl)-4-methyl-1,2,5-oxadiazole-3-carboxamide

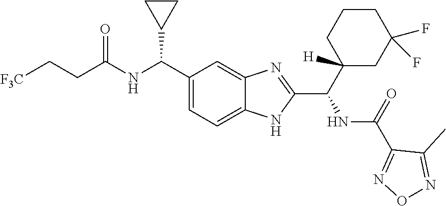 [SMILES: Cc1nonc1C(=O)N[C@H](c1nc2cc([C@H](NC(=O)CCC(F)(F)F)C3CC3)ccc2[nH]1)[C@@H]1CCCC(F)(F)C1]

A solution of N—((S)-(5-((R)-cyclopropyl(4,4,4-trifluorobutanamido)methyl)-1-((2-(trimethylsilyl)ethoxy)methyl)-1H-benzo[d]imidazol-2-yl)((R)-3,3-difluorocyclohexyl)methyl)-4-methyl-1,2,5-oxadiazole-3-carboxamide and N—((S)-(6-((R)-cyclopropyl(4,4,4-trifluorobutanamido)methyl)-1-((2-(trimethylsilyl)ethoxy)methyl)-1H-benzo[d]imidazol-2-yl)((R)-3,3-difluorocyclohexyl)methyl)-4-methyl-1,2,5-oxadiazole-3-carboxamide (16 mg, 0.023 mmol, Intermediate 398) in DCM (1 mL) was treated with TFA (0.005 mL, 0.063 mmol) and the resulting mixture stirred at rt for 2 h. The reaction was quenched with water and then partitioned between saturated aqueous NaHCO₃ and DCM. The organic layer was dried over anhydrous Na₂SO₄, filtered, and concentrated to dryness to afford the crude product, which was purified by silica gel chromatography (0-100% EtOAc/hexanes) to afford the title compound as a white solid. ¹H NMR (500 MHz, DMSO-d₆, benzimidazole NH absent from exchange) δ 9.58 (d, J=8.2 Hz, 1H), 8.58 (d, J=8.3 Hz, 1H), 7.59-7.47 (m, 2H), 7.23 (d, J=8.4 Hz, 1H), 5.32-5.18 (m, 1H), 4.39-4.29 (m, 1H), 2.47 (s, 3H), 2.45-2.32 (m, 2H), 2.02-1.87 (m, 3H), 1.86-1.77 (m, 2H), 1.75-1.60 (m, 2H), 1.49-1.30 (m, 2H), 1.27-1.12 (m, 2H), 0.91-0.75 (m, 1H), 0.57-0.27 (m, 4H). MS (ESI) m/z: [M+H]⁺ Found 569.2.

### Example 264

### N—((S)—((R)-3,3-Difluorocyclohexyl)(5-((R)-1-(4,4,4-trifluorobutanamido)ethyl)-1H-benzo[d]imidazol-2-yl)methyl)-4-methyl-1,2,5-oxadiazole-3-carboxamide

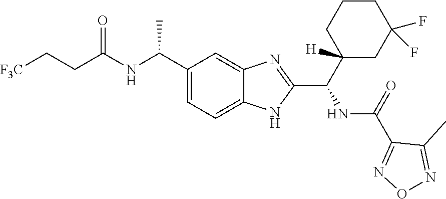 [SMILES: Cc1nonc1C(=O)N[C@H](c1nc2cc([C@@H](C)NC(=O)CCC(F)(F)F)ccc2[nH]1)[C@@H]1CCCC(F)(F)C1]

A solution of the mixture of SEM isomers, N—((S)—((R)-3,3-difluorocyclohexyl)(5-((R)-1-(4,4,4-trifluorobutanamido)ethyl)-1-((2-(trimethylsilyl)ethoxy)methyl)-1H-benzo[d]imidazol-2-yl)methyl)-4-methyl-1,2,5-oxadiazole-3-carboxamide and N—((S)—((R)-3,3-difluorocyclohexyl)(6-((R)-1-((3,3,3-trifluoropropyl)amino)ethyl)-1-((2-(trimethylsilyl)ethoxy)methyl)-1H-benzo[d]imidazol-2-yl)methyl)-4-methyl-1,2,5-oxadiazole-3-carboxamide (139 mg, 0.21 mmol, Intermediate 401) in DCM (2 mL) was treated with TFA (2 mL, 26 mmol). The mixture was stirred at 50° C. for 2 h, cooled to rt, and stirring continued for 17 h. The reaction mixture was diluted with water, neutralized with 1 N aqueous NaOH, and extracted with EtOAc. The EtOAc extract was dried over anhydrous Na₂SO₄, filtered, and concentrated to dryness to give the crude product, which was purified by silica gel chromatography (0-100% EtOAc/hexanes) to afford the title compound as a white solid. ¹H NMR (500 MHz, DMSO-d₆, benzimidazole NH absent from exchange) δ 9.80 (d, J=7.4 Hz, 1H), 8.57 (d, J=7.7 Hz, 1H), 7.69-7.51 (m, 2H), 7.38 (dd, J=8.5, 1.6 Hz, 1H), 5.44-5.35 (m, 1H), 5.12-4.98 (m, 1H), 2.53-2.33 (m, 8H), 2.06-1.90 (m, 2H), 1.90-1.61 (m, 4H), 1.51-1.42 (m, 1H), 1.40 (d, J=7.0 Hz, 3H), 1.34-1.18 (m, 1H). MS (ESI) m/z: [M+H]⁺ Found 543.2.

### Example 265

### N—((S)—((R)-3,3-Difluorocyclohexyl)(5-((R)-1-(4,4,4-trifluorobutanamido)ethyl)-1H-benzo[d]imidazol-2-yl)methyl)-4-methyl-1,2,5-oxadiazole-3-carboxamide

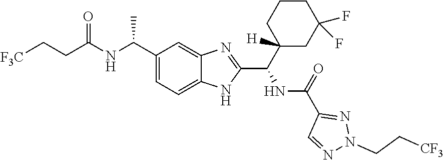 [SMILES: C[C@@H](NC(=O)CCC(F)(F)F)c1ccc2[nH]c([C@@H](NC(=O)c3cnn(CCC(F)(F)F)n3)[C@@H]3CCCC(F)(F)C3)nc2c1]

A solution of the mixture of SEM isomers, N—((S)—((R)-3,3-difluorocyclohexyl)(5-((R)-1-(4,4,4-trifluorobutanamido)ethyl)-1-((2-(trimethylsilyl)ethoxy)methyl)-1H-benzo[d]imidazol-2-yl)methyl)-2-(3,3,3-trifluoropropyl)-2H-1,2,3-triazole-4-carboxamide and N—((S)—((R)-3,3-difluorocyclohexyl)(6-((R)-1-(4,4,4-trifluorobutanamido)ethyl)-1-((2-(trimethylsilyl)ethoxy)methyl)-1H-benzo[d]imidazol-2-yl)methyl)-2-(3,3,3-trifluoropropyl)-2H-1,2,3-triazole-4-carboxamide (186 mg, 0.25 mmol, Intermediate 402) in DCM (2.5 mL) was treated with TFA (0.06 mL, 0.74 mmol), and stirred at rt for 2 h. The temperature was then increased to 70° C. for 2 h, and then returned to rt and stirring continued for 16 h. The reaction mixture was then diluted with water, neutralized with 1 N aqueous NaOH, and extracted with EtOAc. The EtOAc extract was subsequently dried over anhydrous Na₂SO₄, filtered, and concentrated to dryness to give the crude product, which was purified by silica gel chromatography (0-100% EtOAc/hexanes) to afford the title compound as a white solid. ¹H NMR (500 MHz, DMSO-d₆, benzimidazole NH absent from exchange) δ 8.98 (d, J=8.0 Hz, 1H), 8.51 (d, J=7.8 Hz, 1H), 8.29 (s, 1H), 7.66-7.50 (m, 2H), 7.32-7.22 (m, 1H), 5.39-5.25 (m, 1H), 5.08-4.92 (m, 1H), 4.78 (t, J=6.6 Hz, 2H), 3.12-2.95 (m, 2H), 2.49-2.34 (m, 4H), 2.05-1.51 (m, 7H), 1.51-1.35 (m, 4H), 1.31-1.10 (m, 1H). MS (ESI) m/z: [M+H]⁺ Found 624.2.

### Example 266

### 4-Cyclopropyl-N—((S)-(5-((R)-cyclopropyl(2-(3,3-difluorocyclobutyl)acetamido)methyl)-1H-benzo[d]imidazol-2-yl)((1R,5S,6r)-3,3-difluorobicyclo[3.1.0]hexan-6-yl)methyl)-1,2,5-oxadiazole-3-carboxamide

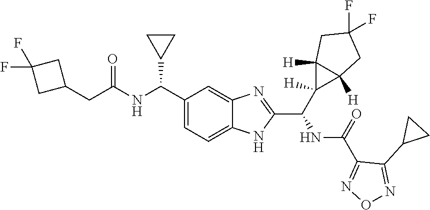 [SMILES: O=C(CC1CC(F)(F)C1)N[C@@H](c1ccc2[nH]c([C@@H](NC(=O)c3nonc3C3CC3)[C@H]3[C@@H]4CC(F)(F)C[C@@H]43)nc2c1)C1CC1]

The title compound was prepared as described for the synthesis of Example 233, using N—((R)-(2-((S)-amino((1R,5S,6r)-3,3-difluorobicyclo[3.1.0]hexan-6-yl)methyl)-1H-benzo[d]imidazol-5-yl)(cyclopropyl)methyl)-2-(3,3-difluorocyclobutyl)acetamide hydrochloride (Intermediate 404) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 4-cyclopropyl-1,2,5-oxadiazole-3-carboxylic acid (Intermediate 78) in place of 4-methyl-1,2,5-oxadiazole-3-carboxylic acid, and purified by silica gel chromatography (0-50% EtOAc/petroleum ether) followed by SFC using a chiral stationary phase (DAICEL CHIRALCEL AD, 10 μm, 250×30 mm, 55% CO₂ in EtOH (0.1% NH₄OH)). The product containing fractions were diluted with water, frozen, and lyophilized to afford the title compound as a white solid. ¹H NMR (400 MHz, DMSO-d₆) δ 12.33 (s, 1H), 9.95 (s, 1H), 8.63-8.41 (m, 1H), 7.67-7.32 (m, 2H), 7.23-7.12 (m, 1H), 4.76-4.57 (m, 1H), 4.33 (t, J=8.4 Hz, 1H), 2.68-2.57 (m, 2H), 2.45-2.23 (m, 8H), 2.22-2.07 (m, 2H), 1.75-1.67 (m, 1H), 1.64-1.56 (m, 1H), 1.52-1.42 (m, 1H), 1.22-1.09 (m, 3H), 1.02-0.95 (m, 2H), 0.55-0.42 (m, 2H), 0.36-0.24 (m, 2H). MS (ESI) m/z: [M+H]⁺ Found 601.2.

### Example 267

### N—((R)-1-(5-((R)-Cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)-4-(2,2,2-trifluoroethoxy)-1,2,5-oxadiazole-3-carboxamide

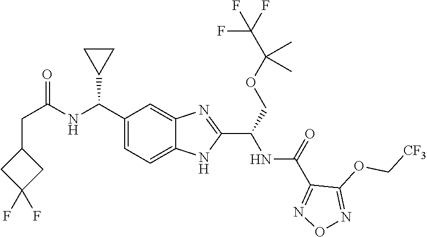 [SMILES: CC(C)(OC[C@H](NC(=O)c1nonc1OCC(F)(F)F)c1nc2cc([C@H](NC(=O)CC3CC(F)(F)C3)C3CC3)ccc2[nH]1)C(F)(F)F]

The title compound was prepared as described for the synthesis of Example 159, using 4-(2,2,2-trifluoroethoxy)-1,2,5-oxadiazole-3-carboxylic acid (Intermediate 226) in place of 4-cyclopropyl-1,2,5-oxadiazole-3-carboxylic acid, and purified by silica gel chromatography (0-100% acetone/hexanes) to afford the title compound as a colorless solid. ¹H NMR (500 MHz, DMSO-d₆) δ 12.44-12.34 (m, 1H), 9.52 (dd, J=11.5, 8.4 Hz, 1H), 8.46 (dd, J=17.3, 8.5 Hz, 1H), 7.61-7.44 (m, 1H), 7.44-7.36 (m, 1H), 7.22-7.11 (m, 1H), 5.49-5.39 (m, 1H), 5.18 (q, J=8.6 Hz, 2H), 4.40-4.30 (m, 1H), 4.21-4.11 (m, 1H), 4.07-3.98 (m, 1H), 2.69-2.56 (m, 2H), 2.45-2.22 (m, 5H), 1.33 (s, 6H), 1.21-1.09 (m, 1H), 0.60-0.43 (m, 2H), 0.35-0.25 (m, 2H). MS (ESI) m/z: [M+H]⁺ Found 683.2.

### Example 268

### 4-Cyclopropyl-N—((R)-1-(5-((R)-cyclopropyl(2-(3,3-difluorocyclobutyl)acetamido)methyl)-1H-benzo[d]imidazol-2-yl)-2-((1,1,1-trifluoro-2-methylpropan-2-yl)oxy)ethyl)isoxazole-3-carboxamide

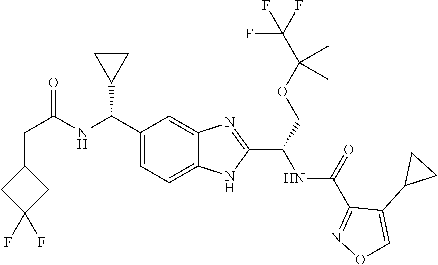 [SMILES: CC(C)(OC[C@H](NC(=O)c1nocc1C1CC1)c1nc2cc([C@H](NC(=O)CC3CC(F)(F)C3)C3CC3)ccc2[nH]1)C(F)(F)F]

The title compound was prepared as described for the synthesis of Example 159, using 4-cyclopropylisoxazole-3-carboxylic acid (Intermediate 223) in place of 4-cyclopropyl-1,2,5-oxadiazole-3-carboxylic acid. The product was purified by silica gel chromatography (0-100% acetone/hexanes) to afford the title compound as a colorless solid. ¹H NMR (500 MHz, DMSO-d₆) δ 12.33 (s, 1H), 8.96 (dd, J=8.6, 4.4 Hz, 1H), 8.83-8.73 (m, 1H), 8.46 (dd, J=18.1, 8.5 Hz, 1H), 7.62-7.48 (m, 1H), 7.46-7.37 (m, 1H), 7.25-7.12 (m, 1H), 5.49-5.37 (m, 1H), 4.43-4.32 (m, 1H), 4.14 (dd, J=9.6, 5.0 Hz, 1H), 4.08-4.00 (m, 1H), 2.72-2.56 (m, 2H), 2.44-2.22 (m, 5H), 2.06-1.96 (m, 1H), 1.33 (d, J=3.8 Hz, 6H), 1.21-1.12 (m, 1H), 0.92-0.83 (m, 2H), 0.69-0.58 (m, 2H), 0.54-0.45 (m, 2H), 0.37-0.26 (m, 2H). MS (ESI) m/z: [M+H]⁺ Found 624.3.

### Example 269

### N-((1R,2R)-1-(6-((S)-(1-Cyanocyclopropyl)(2-((S)-2,2-difluorocyclopropyl)acetamido)methyl)-1H-benzo[d]imidazol-2-yl)-2-(((R)-1,1,1-trifluoropropan-2-yl)oxy)propyl)-4-cyclopropyl-1,2,5-oxadiazole-3-carboxamide

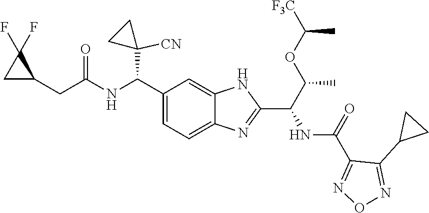 [SMILES: C[C@@H](O[C@H](C)C(F)(F)F)[C@H](NC(=O)c1nonc1C1CC1)c1nc2ccc([C@H](NC(=O)C[C@H]3CC3(F)F)C3(C#N)CC3)cc2[nH]1]

The title compound was prepared as described for the synthesis of Example 234, using N—((S)-(2-((1R,2R)-1-amino-2-(((R)-1,1,1-trifluoropropan-2-yl)oxy)propyl)-1H-benzo[d]imidazol-6-yl)(1-cyanocyclopropyl)methyl)-2-((S)-2,2-difluorocyclopropyl)acetamide (Intermediate 430) in place of N—((R)-(2-((S)-1-amino-4,4-difluoro-3,3-dimethylbutyl)-1H-benzo[d]imidazol-6-yl)(cyclopropyl)methyl)-2-(3,3-difluorocyclobutyl)acetamide and 4-cyclopropyl-1,2,5-oxadiazole-3-carboxylic acid (Intermediate 78) in place of 1-isopropyl-1H-pyrazole-5-carboxylic acid. The crude product was purified by preparative HPLC (Boston Prime C18, 150×30 mm, 5 m column, (55-85% (v/v) CH₃CN in H₂O with 0.05% NH₄OH and 10 mM NH₄HCO₃)) to afford the title compound, after lyophilization, as a white solid. ¹H NMR (400 MHz, DMSO-d₆) δ 8.28 (d, J=6.8 Hz, 1H), 7.80-7.53 (m, 2H), 7.32 (dd, J=1.2, 8.4 Hz, 1H), 6.39 (d, J=8.4 Hz, 1H), 5.50-5.40 (m, 1H), 4.73 (d, J=8.4 Hz, 1H), 4.67-4.56 (m, 1H), 4.30-4.10 (m, 1H), 2.60-2.42 (m, 3H), 2.02-1.85 (m, 1H), 1.52-1.38 (m, 7H), 1.26-1.06 (m, 10H). MS (ESI) m/z: [M+H]⁺ Found 636.4.

### Example 270

### N-((1R,2R)-1-(5-((S)-2-Cyclopropoxy-1-(2-((S)-2,2-difluorocyclopropyl)acetamido)ethyl)-1H-benzo[d]imidazol-2-yl)-2-((1,1,1-trifluoro-2-methylpropan-2-yl)oxy)propyl)-4-methyl-1,2,5-oxadiazole-3-carboxamide

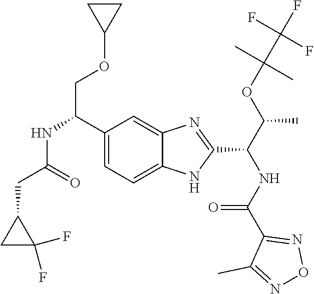 [SMILES: Cc1nonc1C(=O)N[C@H](c1nc2cc([C@@H](COC3CC3)NC(=O)C[C@H]3CC3(F)F)ccc2[nH]1)[C@@H](C)OC(C)(C)C(F)(F)F]

A mixture of N—((S)-1-(2-((1R,2R)-1-amino-2-((1,1,1-trifluoro-2-methylpropan-2-yl)oxy)propyl)-1H-benzo[d]imidazol-5-yl)-2-cyclopropoxyethyl)-2-((S)-2,2-difluorocyclopropyl)acetamide (75.0 mg, 0.145 mmol, Intermediate 449) and triethylamine (0.101 mL, 0.723 mmol) in DCM (1 mL) was stirred at rt for 10 min. Then, 2,5-dioxopyrrolidin-1-yl 4-methyl-1,2,5-oxadiazole-3-carboxylate (102 mg, 0.454 mmol, Intermediate 80) was added and the resulting mixture was stirred at rt for 1 h. The mixture was directly subjected to silica gel chromatography (0-0.2% MeOH/DCM) and then subsequently purified by SFC (DAICEL CHIRALPAK AS column, 10 μm, 250×30 mm; 20% (v/v) EtOH (containing 0.1% of aqueous NH₃)/80% CO₂) to provide the title compound as a white solid (19% yield). ¹H NMR (400 MHz, CDCl₃) δ 8.57-8.47 (m, 1H), 7.65-7.41 (m, 2H), 7.25-7.20 (m, 1H), 6.42-6.30 (m, 1H), 5.39-5.32 (m, 1H), 5.29-5.19 (m, 1H), 4.69-4.57 (m, 1H), 3.87-3.72 (m, 2H), 3.35-3.23 (m, 1H), 2.64 (s, 3H), 2.46-2.42 (m, 2H), 1.95-1.85 (m, 2H), 1.60-1.52 (m, 4H), 1.49 (s, 3H), 1.15-1.08 (m, 1H), 1.07-1.04 (m, 3H), 0.60-0.53 (m, 1H), 0.52-0.43 (m, 3H). MS (ESI) m/z: [M+H]⁺ Found 629.3.

### Example 271

### N-((1R,2R)-1-(5-((S)-2-Cyclopropoxy-1-(2-(3,3-difluorocyclobutyl)acetamido)ethyl)-1H-benzo[d]imidazol-2-yl)-2-((1,1,1-trifluoro-2-methylpropan-2-yl)oxy)propyl)-4-methyl-1,2,5-oxadiazole-3-carboxamide

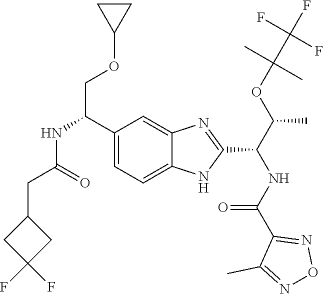 [SMILES: Cc1nonc1C(=O)N[C@H](c1nc2cc([C@@H](COC3CC3)NC(=O)CC3CC(F)(F)C3)ccc2[nH]1)[C@@H](C)OC(C)(C)C(F)(F)F]

The title compound was prepared as described for the synthesis of Example 270 using N—((S)-1-(2-((1R,2R)-1-amino-2-((1,1,1-trifluoro-2-methylpropan-2-yl)oxy)propyl)-1H-benzo[d]imidazol-5-yl)-2-cyclopropoxyethyl)-2-(3,3-difluorocyclobutyl)acetamide (Intermediate 452) in place of N—((S)-1-(2-((1R,2R)-1-amino-2-((1,1,1-trifluoro-2-methylpropan-2-yl)oxy)propyl)-1H-benzo[d]imidazol-5-yl)-2-cyclopropoxyethyl)-2-((S)-2,2-difluorocyclopropyl)acetamide. The material was initially purified by silica gel chromatography (0-0.5% MeOH/DCM) and then subsequently purified by SFC (DAICEL CHIRALPAK AD column, 10 μm, 250×30 mm; 30% (v/v) EtOH (containing 0.1% of aqueous NH₃)/70% CO₂) to provide the title compound as a white solid (41% yield). ¹H NMR (400 MHz, CDCl₃) δ 9.66-9.49 (m, 1H), 8.55-8.44 (m, 1H), 7.73-7.66 (m, 1H), 7.44-7.37 (m, 1H), 7.25-7.17 (m, 1H), 6.20-6.15 (m, 1H), 5.37-5.30 (m, 1H), 5.28-5.21 (m, 1H), 4.68-4.59 (m, 1H), 3.88-3.73 (m, 2H), 3.33-3.26 (m, 1H), 2.82-2.70 (m, 2H), 2.64 (s, 3H), 2.60-2.51 (m, 1H), 2.48-2.44 (m, 2H), 2.33-2.21 (m, 2H), 1.62-1.60 (m, 3H), 1.50 (s, 3H), 1.06-1.02 (m, 3H), 0.61-0.53 (m, 1H), 0.52-0.44 (m, 3H). MS (ESI) m/z: [M+H]⁺ Found 643.4.

### Example 272

### N-((1R,2R)-1-(5-((S)-2-Cyclopropoxy-1-(2-(3,3-difluorocyclobutyl)acetamido)ethyl)-1H-benzo[d]imidazol-2-yl)-2-(((S)-1,1,1-trifluoropropan-2-yl)oxy)propyl)-4-methyl-1,2,5-oxadiazole-3-carboxamide

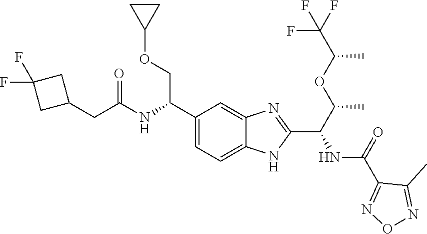 [SMILES: Cc1nonc1C(=O)N[C@H](c1nc2cc([C@@H](COC3CC3)NC(=O)CC3CC(F)(F)C3)ccc2[nH]1)[C@@H](C)O[C@@H](C)C(F)(F)F]

The title compound was prepared as described for the synthesis of Example 270 using N—((S)-1-(2-((1R,2R)-1-amino-2-(((S)-1,1,1-trifluoropropan-2-yl)oxy)propyl)-1H-benzo[d]imidazol-5-yl)-2-cyclopropoxyethyl)-2-(3,3-difluorocyclobutyl)acetamide (Intermediate) in place of N—((S)-1-(2-((1R,2R)-1-amino-2-((1,1,1-trifluoro-2-methylpropan-2-yl)oxy)propyl)-1H-benzo[d]imidazol-5-yl)-2-cyclopropoxyethyl)-2-((S)-2,2-difluorocyclopropyl)acetamide. The material was initially purified by silica gel chromatography (0-5% MeOH/DCM) and then subsequently purified by SFC (DAICEL CHIRALPAK AD column, 10 μm, 250×30 mm; 5-25% (v/v) MeOH (containing 0.1% of aqueous NH₃)/CO₂) to provide the title compound as a white solid (14% yield). ¹H NMR (400 MHz, CDCl₃) δ 9.51 (br s, 1H), 8.37-8.26 (m, 1H), 7.73-7.65 (m, 1H), 7.46-7.36 (m, 1H), 7.25-7.18 (m, 1H), 6.18-6.09 (m, 1H), 5.50-5.40 (m, 1H), 5.28-5.19 (m, 1H), 4.52-4.40 (m, 1H), 4.29-4.16 (m, 1H), 3.85-3.75 (m, 2H), 3.36-3.25 (m, 1H), 2.83-2.71 (m, 2H), 2.64 (s, 3H), 2.59-2.50 (m, 1H), 2.48-2.41 (m, 2H), 2.30-2.20 (m, 2H), 1.45-1.42 (m, 3H), 1.13-1.10 (m, 3H), 0.61-0.53 (m, 1H), 0.50-0.41 (m, 3H). MS (ESI) m/z: [M+H]⁺ Found 629.3.

### Example 273

### N-((1R,2R)-1-(5-((S)-2-Cyclopropoxy-1-(2-(3,3-difluorocyclobutyl)acetamido)ethyl)-1H-benzo[d]imidazol-2-yl)-2-(((S)-1,1,1-trifluoropropan-2-yl)oxy)propyl)-1-isopropyl-1H-pyrazole-5-carboxamide

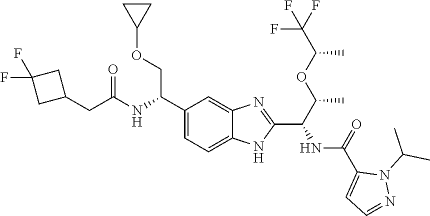 [SMILES: CC(C)n1nccc1C(=O)N[C@H](c1nc2cc([C@@H](COC3CC3)NC(=O)CC3CC(F)(F)C3)ccc2[nH]1)[C@@H](C)O[C@@H](C)C(F)(F)F]

A mixture of 1-isopropyl-1H-pyrazole-5-carboxylic acid (81 mg, 0.53 mmol), T3P (673 mg, 1.06 mmol, 50% in THF) and DIPEA (0.175 mL, 1.06 mmol) in DCM (1 mL) was stirred at 25° C. for 1 h. Then, N—((S)-1-(2-((1R,2R)-1-amino-2-(((S)-1,1,1-trifluoropropan-2-yl)oxy)propyl)-1H-benzo[d]imidazol-5-yl)-2-cyclopropoxyethyl)-2-(3,3-difluorocyclobutyl)acetamide (137 mg, 0.26 mmol, Intermediate 461) was added and the reaction mixture was stirred at 25° C. for 5 h. The mixture was diluted with H₂O (15 mL) and extracted with DCM (30 mL). The organic layer was washed sequentially with H₂O (10 mL) and brine (10 mL), dried over anhydrous Na₂SO₄, filtered, and concentrated to dryness. The resulting residue was purified initially by silica gel chromatography (0-10% MeOH/DCM) and then subsequently purified by SFC (DAICEL CHIRALCEL OD column, 10 μm, 250×30 mm; 15% (v/v) EtOH (containing 0.1% of aqueous NH₃)/85% CO₂) to provide the title compound as a white solid (15% yield). ¹H NMR (400 MHz, DMSO-d₆) δ 12.46-12.33 (m, 1H), 8.76-8.61 (m, 1H), 8.45-8.35 (m, 1H), 7.55-7.48 (m, 2H), 7.45-7.38 (m, 1H), 7.18-7.09 (m, 1H), 6.90-6.85 (m, 1H), 5.46-5.34 (m, 1H), 5.16-5.04 (m, 1H), 4.43-4.23 (m, 2H), 3.62-3.55 (m, 2H), 3.31-3.28 (m, 1H), 2.69-2.56 (m, 2H), 2.44-2.22 (m, 5H), 1.40-1.29 (m, 6H), 1.27-1.22 (m, 4H), 1.14-1.06 (m, 3H), 0.49-0.35 (m, 4H). MS (ESI) m/z: [M+H]⁺ Found 655.2.

### Example 274

### N-((1R,2R)-1-(5-((S)-2-Cyclopropoxy-1-(2-(3,3-difluorocyclobutyl)acetamido)ethyl)-1H-benzo[d]imidazol-2-yl)-2-(((S)-1,1,1-trifluoropropan-2-yl)oxy)propyl)-4-methyl-1,2,5-oxadiazole-3-carboxamide

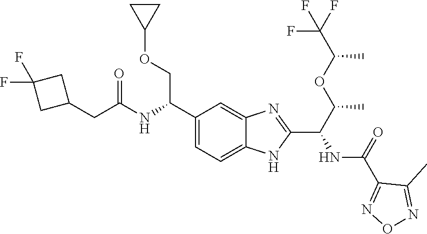 [SMILES: Cc1nonc1C(=O)N[C@H](c1nc2cc([C@@H](COC3CC3)NC(=O)CC3CC(F)(F)C3)ccc2[nH]1)[C@@H](C)O[C@@H](C)C(F)(F)F]

The title compound was prepared as described for the synthesis of Example 270 using N—((S)-1-(2-((1R,2R)-1-amino-2-(((R)-1,1,1-trifluoropropan-2-yl)oxy)propyl)-1H-benzo[d]imidazol-5-yl)-2-cyclopropoxyethyl)-2-(3,3-difluorocyclobutyl)acetamide trifluoroacetate (Intermediate 469) in place of N—((S)-1-(2-((1R,2R)-1-amino-2-((1,1,1-trifluoro-2-methylpropan-2-yl)oxy)propyl)-1H-benzo[d]imidazol-5-yl)-2-cyclopropoxyethyl)-2-((S)-2,2-difluorocyclopropyl)acetamide. The material was purified initially by silica gel chromatography (0-5% MeOH/DCM) and then subsequently purified by SFC (DAICEL CHIRALCEL OD column, 10 μm, 250×30 mm; 30% (v/v) MeOH (containing 0.1% of aqueous NH₃)/70% CO₂) to provide the title compound as a white solid (15% yield). ¹H NMR (400 MHz, CDCl₃, benzimidazole NH is absent) δ 8.30 (d, J=6.8 Hz, 1H), 7.53 (s, 2H), 7.22 (dd, J=1.2, 8.4 Hz, 1H), 6.21 (d, J=7.2 Hz, 1H), 5.45-5.38 (m, 1H), 5.28-5.19 (m, 1H), 4.63-4.53 (m, 1H), 4.24-4.12 (m, 1H), 3.85-3.71 (m, 2H), 3.34-3.26 (m, 1H), 2.83-2.69 (m, 2H), 2.65 (s, 3H), 2.61-2.51 (m, 1H), 2.49-2.44 (m, 2H), 2.34-2.19 (m, 2H), 1.38 (d, J=6.8 Hz, 3H), 1.16 (d, J=6.4 Hz, 3H), 0.60-0.41 (m, 4H). MS (ESI) m/z: [M+H]⁺ Found 629.2.

### Example 275

### N-((1R,2R)-1-(5-((S)-2-Cyclopropoxy-1-(2-((S)-2,2-difluorocyclopropyl)acetamido)ethyl)-1H-benzo[d]imidazol-2-yl)-2-(((R)-1,1,1-trifluoropropan-2-yl)oxy)propyl)-4-methyl-1,2,5-oxadiazole-3-carboxamide

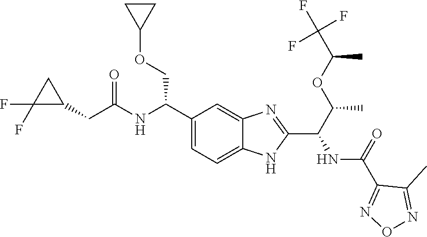

The title compound was prepared as described for the synthesis of Example 270 using N—((S)-1-(2-((1R,2R)-1-amino-2-(((R)-1,1,1-trifluoropropan-2-yl)oxy)propyl)-1H-benzo[d]imidazol-5-yl)-2-cyclopropoxyethyl)-2-((S)-2,2-difluorocyclopropyl)acetamide trifluoroacetate (Intermediate 472) in place of N—((S)-1-(2-((1R,2R)-1-amino-2-((1,1,1-trifluoro-2-methylpropan-2-yl)oxy)propyl)-1H-benzo[d]imidazol-5-yl)-2-cyclopropoxyethyl)-2-((S)-2,2-difluorocyclopropyl)acetamide. The material was purified initially by silica gel chromatography (0-0.5% MeOH/DCM) and then subsequently purified by SFC (DAICEL CHIRALCEL OD column, 10 μm, 250×30 mm; 35% (v/v) MeOH (containing 0.1% of aqueous NH₃)/65% CO₂) to provide the title compound as a white solid (9% yield). ¹H NMR (400 MHz, CDCl₃) δ 8.27 (d, J=6.8 Hz, 1H), 7.84-7.32 (m, 2H), 7.23 (d, J=8.4 Hz, 1H), 6.31 (d, J=7.2 Hz, 1H), 5.45-5.36 (m, 1H), 5.29-5.21 (m, 1H), 4.63-4.52 (m, 1H), 4.27-4.12 (m, 1H), 3.87-3.74 (m, 2H), 3.35-3.25 (m, 1H), 2.65 (s, 3H), 2.43 (d, J=7.2 Hz, 2H), 1.96-1.82 (m, 1H), 1.55-1.50 (m, 2H), 1.38 (d, J=6.4 Hz, 3H), 1.20-1.04 (m, 4H), 0.62-0.40 (m, 4H). MS (ESI) m/z: [M+H]+ Found 615.3.

### Example 276

### N-((1R,2R)-2-Cyclopropoxy-1-(5-((S)-2-cyclopropoxy-1-(2-(3,3-difluorocyclobutyl)acetamido)ethyl)-1H-benzo[d]imidazol-2-yl)propyl)-4-cyclopropyl-1,2,5-oxadiazole-3-carboxamide

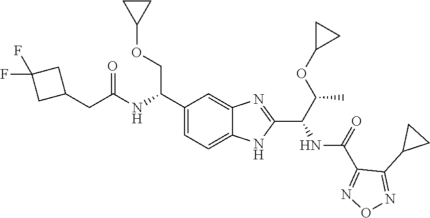 [SMILES: C[C@@H](OC1CC1)[C@H](NC(=O)c1nonc1C1CC1)c1nc2cc([C@@H](COC3CC3)NC(=O)CC3CC(F)(F)C3)ccc2[nH]1]

A mixture of N—((S)-1-(2-((1R,2R)-1-amino-2-cyclopropoxypropyl)-1H-benzo[d]imidazol-5-yl)-2-cyclopropoxyethyl)-2-(3,3-difluorocyclobutyl)acetamide trifluoroacetate (112 mg, 0.242 mmol, Intermediate 481), DIPEA (0.127 mL, 0.726 mmol) and 2,5-dioxopyrrolidin-1-yl 4-cyclopropyl-1,2,5-oxadiazole-3-carboxylate (182 mg, 0.726 mmol, Intermediate 482) in DCM (3 mL) was stirred at rt for 16 h. The mixture was directly subjected to silica gel chromatography (0-50% EtOAc/petroleum ether) and then subsequently purified by SFC (DAICEL CHIRALPAK OD-H column, 5 μm, 250×30 mm; 20% (v/v) EtOH (containing 0.1% of aqueous NH₃)/80% CO₂) to afford the title compound as a white solid (13% yield). ¹H NMR (400 MHz, DMSO-d₆) δ 9.26 (d, J=8.4 Hz, 1H), 8.42 (d, J=8.4 Hz, 1H), 7.54-7.40 (m, 2H), 7.20-7.06 (m, 1H), 5.34-5.24 (m, 1H), 5.16-5.03 (m, 1H), 4.25-4.17 (m, 1H), 3.62-3.59 (m, 1H), 3.32-3.30 (m, 1H), 2.71-2.54 (m, 4H), 2.48-2.22 (m, 7H), 1.19-1.10 (m, 5H), 1.01-0.95 (m, 2H), 0.48-0.31 (m, 8H). MS (ESI) m/z: [M+H]⁺ Found 599.3.

### In Vitro Biological Data

### IL-17A(FLAG-Tagged): IL-17RA(His-Tagged) Binding Disruption Eu-HTRF Assay

An antibody directed against the FLAG tag of IL-17A (SEQ ID NO: 1) is labeled with the HTRF donor chromophore (Europium-cryptate). IL-17A is present as a dimer that is “locked into” this quaternary structure due to the formation of loop-spanning intramolecular disulfide bridges. The construct of IL-17RA used in the assay excludes the outer-membrane portion of the receptor and is fused to a C-terminal 10×His tag (SEQ ID NO:2). An antibody directed against the His tag of the IL-17RA chimera is labeled with the HTRF acceptor chromophore (“D2”). The fluorescence-resonance energy transfer (FRET) depends on the vicinity of the donor chromophore to the acceptor, and interruption of the binding between the IL-17A and IL-17RA causes the reduction/loss of FRET. Therefore, this assay allows to evaluate the compound effect on the binding IL-17A and IL-17RA by monitoring the fluorescence intensity of donor vs acceptor. The assay was run using either Protocol 1 or Protocol 2 as described below.

Protocol 1. 40 nl of 2-fold serial diluted compound solution for total 22 dilution points is added into each well of a 1536-well, white, low-volume, non-binding plate (Greiner #782904), then 2 μl of FLAG tagged IL-17A at 2× final concentration (2.5 nM) in solution of PBS+0.01% Triton-X100 is added to each well. The assay plate is briefly centrifuged then incubated for 1 h at rt. A mixed solution is prepared containing 2×5 nM 10HIS×IL-17RA, 2×2.5 nM Eu-anti-FLAG (CISBIO), 2×5 nM D2-anti-HIS (CISBIO) in PBS+0.01% Triton-X100+200 mM Potassium Fluoride (Sigma 60238) and 2 μl of mix is added to each well of the assay plate. The plate is briefly centrifuged then incubated for 2 h at rt. The HTRF intensities at the wavelength of donor (620 nm) and acceptor (665 nm) are measured using BMG Pherastar. The ratio between intensities at two wavelengths is calculated and plotted against the compound concentration and the data is fitted to a one-site competition model to yield IC₅₀ of the compound.

Protocol 2. 40 nl of 2-fold serial diluted compound solution for total 22 dilution points is added into each well of a 1536-well, white, low-volume, non-binding plate (Greiner #782904), then 2 μl of FLAG tagged IL-17A at 2× final concentration (1 nM) in solution of PBS+0.01% Triton-X100 is added to each well. The assay plate is briefly centrifuged then incubated for 1 h at rt. A mixed solution is prepared containing 2×5 nM 10HIS×IL-17RA, 2×2.5 nM Eu-anti-FLAG (CISBIO), 2×5 nM D2-anti-HIS (CISBIO) in PBS+0.01% Triton-X100+200 mM Potassium Fluoride (Sigma 60238) and 2 μl of mix is added to each well of the assay plate. The plate is briefly centrifuged then incubated for 2 h at rt. The HTRF intensities at the wavelength of donor (615 nm) and acceptor (665 nm) are measured using BMG Pherastar. The ratio between intensities at two wavelengths is calculated and plotted against the compound concentration and the data is fitted to a one-site competition model to yield IC₅₀ of the compound.

IL-17A acts directly on keratinocytes through binding to dimeric receptor IL-17RA/RC and drives the production of a number of inflammatory mediators known to be elevated in psoriasis lesional tissue. IL-17A small molecule inhibitors that block the IL-17A to interact with IL-17R would inhibit the IL-17A signaling in its targeted cells such as keratinocytes. The compound functional activity is evaluated for its impact on IL-17A-induced G-CSF production in human normal keratinocyte (NHK).

### NHK Assay

Adult normal human keratinocytes are cultured in keratinocyte growth medium (Lonza) in a flask till reaching ˜90% confluence, then cells are transferred to a 384-well plate at density of 3000-4000 cell/well. Recombinant human IL-17A (Gibco PHC9174) is pre-incubated with titrated compound or DMSO for 1 h at rt then added to the cell culture plate. The final concentration of IL-17A is 5 ng/mL and DMSO is 0.2%, in the culture containing 5% FBS. Cells are cultured/treated for 24 h at 37° C. Supernatants are collected and G-CSF production is measured through HTRF technology using Human G-CSF Kit (CisBio). G-CSF concentration was extrapolated from the standard curve and IC₅₀ is determined using GraphPad Prism. Cell viability is also evaluated using CellTiter-Glo kit (Promega) and effect of compound on cell viability is compared to DMSO control.

In cases where the compound was tested more than once, the IC₅₀ value shown is a simple average of the measured values.

A: IC₅₀<0.05 μM; B: 0.05 μM≤IC₅₀≤0.1 μM; C: IC₅₀>0.1 μM
Not available

| | IL-17A | IL-17A | |
|---|---|---|---|
| | HTRF | HTRF | |
| Example | Assay | Assay | NHK |
| # | Protocol 1 | Protocol 2 | Assay |
| 1 | C | — | C |
| 2 | C | — | C |
| 3 | C | — | C |
| 4 | C | — | B |
| 5 | C | — | C |
| 6 | C | — | C |
| 7 | C | — | C |
| 8 | C | — | C |
| 9 | C | — | C |
| 10 | C | — | C |
| 11 | C | — | C |
| 12 | B | — | C |
| 13 | C | — | C |
| 14 | C | — | C |
| 15 | C | — | C |
| 16 | C | — | C |
| 17 | C | — | C |
| 18 | C | — | C |
| 19 | C | — | B |
| 20 | C | — | B |
| 21 | C | — | C |
| 22 | C | — | C |
| 23 | C | — | C |
| 24 | C | — | C |
| 25 | C | — | C |
| 26 | C | — | C |
| 27 | C | — | C |
| 28 | C | — | C |
| 29 | C | — | C |
| 30 | B | — | C |
| 31 | C | — | C |
| 32 | C | — | C |
| 33 | C | — | C |
| 34 | C | — | B |
| 35 | C | — | C |
| 36 | C | — | B |
| 37 | C | — | C |
| 38 | C | — | C |
| 39 | C | — | C |
| 40 | C | — | C |
| 41 | C | — | C |
| 42 | C | — | A |
| 43 | C | — | C |
| 44 | C | — | C |
| 45 | C | — | B |
| 46 | C | — | C |
| 47 | C | — | A |
| 48 | C | — | C |
| 49 | C | — | A |
| 50 | C | — | C |
| 51 | C | — | B |
| 52 | C | — | C |
| 53 | C | — | B |
| 54 | C | — | A |
| 55 | C | — | C |
| 56 | B | — | B |
| 57 | A | — | A |
| 58 | C | — | C |
| 59 | C | — | C |
| 60 | C | — | C |
| 61 | C | — | C |
| 62 | C | — | C |
| 63 | B | — | B |
| 64 | C | — | C |
| 65 | C | — | C |
| 66 | C | — | B |
| 67 | C | — | C |
| 68 | C | — | C |
| 69 | C | — | C |
| 70 | C | — | C |
| 71 | C | — | C |
| 72 | C | — | C |
| 73 | C | — | C |
| 74 | C | — | C |
| 75 | C | — | C |
| 76 | A | — | A |
| 77 | B | — | B |
| 78 | C | — | C |
| 79 | C | — | C |
| 80 | C | — | B |
| 81 | C | — | C |
| 82 | C | — | C |
| 83 | C | — | C |
| 84 | C | — | C |
| 85 | C | — | C |
| 86 | C | — | C |
| 87 | C | — | C |
| 88 | C | — | C |
| 89 | C | — | C |
| 90 | C | — | C |
| 91 | C | — | C |
| 92 | C | — | C |
| 93 | C | — | C |
| 94 | C | — | C |
| 95 | C | — | C |
| 96 | C | — | C |
| 97 | C | — | C |
| 98 | C | — | C |
| 99 | C | — | C |
| 100 | C | — | C |
| 101 | C | — | C |
| 102 | C | — | C |
| 103 | C | — | C |
| 104 | C | — | C |
| 105 | C | — | C |
| 106 | C | — | C |
| 107 | C | — | C |
| 108 | C | — | C |
| 109 | C | — | C |
| 110 | C | — | C |
| 111 | C | — | C |
| 112 | C | — | C |
| 113 | C | — | C |
| 114 | C | — | C |
| 115 | C | — | C |
| 116 | B | — | A |
| 117 | C | — | C |
| 118 | C | — | B |
| 119 | C | — | A |
| 120 | C | — | B |
| 121 | C | — | A |
| 122 | C | C | A |
| 123 | C | — | C |
| 124 | C | — | C |
| 125 | C | — | A |
| 126 | C | — | B |
| 127 | B | — | A |
| 128 | C | — | C |
| 129 | C | — | B |
| 130 | C | — | C |
| 131 | C | — | C |
| 132 | C | — | C |
| 133 | C | — | C |
| 134 | C | — | C |
| 135 | C | — | A |
| 136 | C | — | C |
| 137 | C | — | B |
| 138 | C | — | B |
| 139 | C | — | A |
| 140 | C | — | C |
| 141 | A | — | A |
| 142 | B | — | A |
| 143 | A | — | A |
| 144 | C | — | A |
| 145 | C | — | C |
| 146 | C | — | C |
| 147 | B | — | A |
| 148 | C | — | C |
| 149 | C | — | A |
| 150 | C | — | C |
| 151 | A | B | C |
| 152 | A | A | A |
| 153 | A | — | C |
| 154 | A | — | B |
| 155 | A | — | C |
| 156 | B | — | B |
| 157 | A | — | B |
| 158 | A | — | C |
| 159 | A | — | A |
| 160 | A | — | A |
| 161 | A | — | B |
| 162 | C | — | C |
| 163 | C | — | C |
| 164 | A | A | A |
| 165 | C | — | C |
| 166 | A | — | A |
| 167 | B | — | B |
| 168 | B | — | C |
| 169 | A | — | A |
| 170 | A | — | A |
| 171 | B | — | A |
| 172 | C | — | A |
| 173 | C | — | C |
| 174 | C | — | B |
| 175 | A | — | C |
| 176 | A | — | C |
| 177 | C | — | A |
| 178 | A | — | A |
| 179 | A | — | C |
| 180 | B | — | C |
| 181 | A | — | A |
| 182 | A | — | A |
| 183 | A | — | C |
| 184 | B | — | C |
| 185 | A | — | C |
| 186 | A | — | B |
| 187 | — | — | C |
| 188 | C | — | C |
| 189 | C | — | C |
| 190 | A | — | A |
| 191 | C | — | C |
| 192 | C | — | C |
| 193 | A | — | B |
| 194 | C | — | C |
| 195 | C | — | C |
| 196 | C | — | C |
| 197 | A | — | B |
| 198 | B | — | C |
| 199 | C | — | C |
| 200 | B | — | B |
| 201 | C | — | C |
| 202 | A | — | A |
| 203 | C | — | C |
| 204 | A | — | A |
| 205 | A | — | B |
| 206 | B | — | A |
| 207 | B | — | C |
| 208 | C | — | C |
| 209 | B | — | A |
| 210 | C | — | C |
| 211 | C | — | C |
| 212 | C | — | C |
| 213 | A | — | A |
| 214 | B | — | A |
| 215 | C | — | C |
| 216 | C | — | C |
| 217 | C | — | C |
| 218 | C | — | C |
| 219 | C | — | C |
| 220 | B | — | C |
| 221 | C | — | C |
| 222 | C | — | C |
| 223 | C | — | C |
| 224 | C | — | C |
| 225 | C | — | C |
| 226 | C | — | C |
| 227 | C | — | C |
| 228 | C | — | C |
| 229 | C | — | C |
| 230 | C | — | B |
| 231 | C | — | C |
| 232 | C | — | B |
| 233 | A | — | C |
| 234 | B | — | B |
| 235 | C | — | C |
| 236 | C | — | C |
| 237 | A | — | B |
| 238 | A | — | A |
| 239 | A | — | A |
| 240 | A | — | C |
| 241 | B | — | A |
| 242 | C | — | C |
| 243 | C | — | B |
| 244 | C | — | C |
| 245 | C | — | C |
| 246 | A | — | A |
| 247 | A | — | A |
| 248 | B | — | C |
| 249 | C | — | C |
| 250 | B | — | B |
| 251 | C | — | C |
| 252 | C | — | C |
| 253 | C | — | C |
| 254 | A | — | C |
| 255 | B | — | A |
| 256 | C | — | C |
| 257 | C | — | C |
| 258 | B | — | B |
| 259 | B | — | B |
| 260 | C | — | C |
| 261 | C | — | B |
| 262 | C | — | A |
| 263 | A | — | B |
| 264 | A | — | C |
| 265 | C | — | C |
| 266 | A | — | A |
| 267 | A | — | A |
| 268 | A | — | B |
| 269 | A | — | A |
| 270 | — | B | B |
| 271 | — | B | A |
| 272 | — | C | A |
| 273 | — | C | A |
| 274 | — | B | B |
| 275 | — | B | B |
| 276 | — | B | A |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All documents cited herein are incorporated by reference.

Name: IL-17A-Flag
SEQ ID NO: 1
MATGSRTSLLLAFGLLCLPWLQEGSAGSDYKDDDDKGSGSGSLEVLFQGP
GITIPRNPGCPNSEDKNFPRTVMVNLNIHNRNTNTNPKRSSDYYNRSTSP
WNLHRNEDPERYPSVIWEAQCRHLGCINADGNVDYHMNSVPIQQEILVLR
REPPHCPNSFRLEKILVSVGCTCVTPIVHHVQ
Name: IL-17RA
SEQ ID NO: 2
MKFLVNVALVFMVVYISYIYALRLLDHRALVCSQPGLNCTVKNSTCLDDS
WIHPRNLTPSSPKDLQIQLHFAHTQQGDLFPVAHIEWTLQTDASILYLEG
AELSVLQLNTNERLCVRFEFLSKLRHHHRRWRFTFSHFVVDPDQEYEVTV
HHLPKPIPDGDPNHQSKNFLVPDCEHARMKVTTPCMSSGSLWDPNITVET
LEAHQLRVSFTLWNESTHYQILLTSFPHMENHSCFEHMHHIPAPRPEEFH
QRSNVTLTLRNLKGCCRHQVQIQPFFSSCLNDCLRHSATVSCPEMPDTPE
PIPDYMPLWGSGGHHHHHHHHHH*

1. A compound of Formula (I): 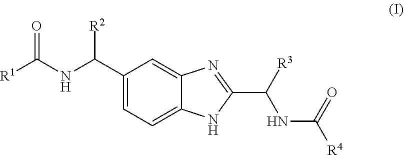
or a pharmaceutically acceptable salt thereof, wherein:
R¹ is —C₍₁₋₆₎)alkyl, —C₍₁₋₃₎alkyl-C₍₃₋₆₎cycloalkyl, or —C₍₁₋₃₎alkyl-C₍₅₋₁₀₎polycycloalkyl, each of which is unsubstituted or substituted with one to six R^{1a} groups;
each R^{1a} independently for each occurrence is fluorine, —C₍₁₋₃₎alkyl, —C₍₃₋₅₎cycloalkyl, —CN, —OH, —O—C₍₁₋₃₎alkyl, or —O—C₍₃₋₄₎cycloalkyl, wherein the —C₍₁₋₃₎alkyl, —C₍₃₋₅₎cycloalkyl, —O—C₍₁₋₃₎alkyl, and —O—C₍₃₋₄₎cycloalkyl groups are unsubstituted or substituted with one to three fluorine atoms;
R² is —C₍₁₋₆₎)alkyl, —C₍₃₋₅₎cycloalkyl, —C₍₁₋₃₎alkyl-C₍₃₋₅₎cycloalkyl, —C₍₁₋₃₎alkyl-O—C₍₁₋₃₎alkyl, C₍₁₋₃₎alkyl-O—C₍₃₋₅₎cycloalkyl, or 4- to 6-membered heterocyclyl, each of which is unsubstituted or substituted with one to six R^{2a} groups;
each R^{2a} independently for each occurrence is fluorine, —C₍₁₋₃₎alkyl, —C₍₃₋₅₎cycloalkyl, —CN, —OH, —O—C₍₁₋₃₎alkyl, or —O—C₍₃₋₄₎cycloalkyl, wherein the —C₍₁₋₃₎alkyl, C₍₃₋₅₎cycloalkyl, —O—C₍₁₋₃₎alkyl, and —O—C₍₃₋₄₎cycloalkyl groups are unsubstituted or substituted with one to three fluorine atoms;
R³ is —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, or —C₍₃₋₄₎cycloalkylC₍₁₋₃₎alkyl, wherein the —C₍₃₋₆₎alkyl, —C₍₃₋₆₎cycloalkyl, —C₍₅₋₁₀₎polycycloalkyl, 3- to 6-membered heterocyclyl, 6- to 10-membered polyheterocyclyl, —C₍₁₋₂₎alkyl-O—C₍₁₋₅₎alkyl, —C₍₁₋₂₎alkylC₍₃₋₆₎cycloalkyl, —C₍₁₋₂₎alkyl-O—C₍₃₋₆₎cycloalkyl, —C₍₁₋₂₎alkyl-C₍₅₋₁₀₎polycycloalkyl, and —C₍₃₋₄₎cycloalkylC₍₁₋₃₎alkyl are unsubstituted or substituted with one to four R^{3a} groups;
each R^{3a} independently for each occurrence is fluorine, —CH₃, —CH₂F, —CHF₂, —CF₃, —O—C₍₁₋₃₎alkyl, —OH, or oxo;
R⁴ is —C₍₁₋₆₎)alkyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl,
wherein the C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups,
wherein the phenyl is unsubstituted or substituted with one to three R^{4b} groups,
wherein the 5-membered heteroaryl is unsubstituted or substituted with one to three R^{4c} groups;
each R^{4a} independently for each occurrence is fluorine, —C₍₁₋₃₎alkyl, or —CN, wherein the —C₍₁₋₃₎alkyl is unsubstituted or substituted with one to three fluorine atoms;
each R^{4b} independently for each occurrence is fluorine or —CN;
each R^{4c} independently for each occurrence is fluorine, —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, —O—C₍₁₋₃₎alkyl, —C(O)NH₂, —CN, or —OH, wherein the —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —C₍₁₋₃₎alkyl-O—C₍₁₋₃₎alkyl-C₍₃₋₆₎cycloalkyl, and —O—C₍₁₋₃₎alkyl, are unsubstituted or substituted with one to six substituents independently selected from the group consisting of fluorine, —OH, and —CN;
alternatively, two R^{4c} groups attached to vicinal atoms on the same ring can be combined with the atoms to which they are attached to form a C₍₃₋₆₎cycloalkyl or a 3- to 6-membered heterocyclyl.

2-5. (canceled)

6. The compound of claim 1, or a pharmaceutically acceptable salt thereof, wherein:
R¹ is —C₍₁₋₆₎)alkyl, —C₍₁₋₃₎alkyl-C₍₃₋₆₎cycloalkyl, or —C₍₁₋₃₎alkyl-C₍₅₋₁₀₎polycycloalkyl, each of which is substituted with one to six R^{1a} groups.

7. The compound of claim 1, or a pharmaceutically acceptable salt thereof, wherein R¹ is:

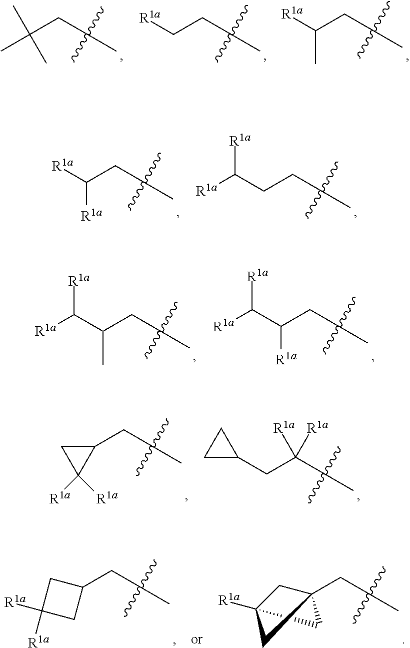

8. The compound of claim 1, or a pharmaceutically acceptable salt thereof, wherein each R^{1a} independently for each occurrence is fluorine, —CH₂F, —CHF₂, or —CF₃.

9-11. (canceled)

12. The compound of claim 1, or a pharmaceutically acceptable salt thereof, wherein R² is —C₍₁₋₄₎alkyl, —C₍₃₋₄₎cycloalkyl, —CH₂—C₍₃₋₄₎cycloalkyl, —C₍₁₋₂₎alkyl-O—C₍₁₋₂₎alkyl, C₍₁₋₂₎alkyl-O—C₍₃₋₄₎cycloalkyl, or tetrahydropyranyl, wherein the —C₍₃₋₄₎cycloalkyl is unsubstituted or substituted with one —CN.

13. The compound of claim 1, or a pharmaceutically acceptable salt thereof, wherein R² is:

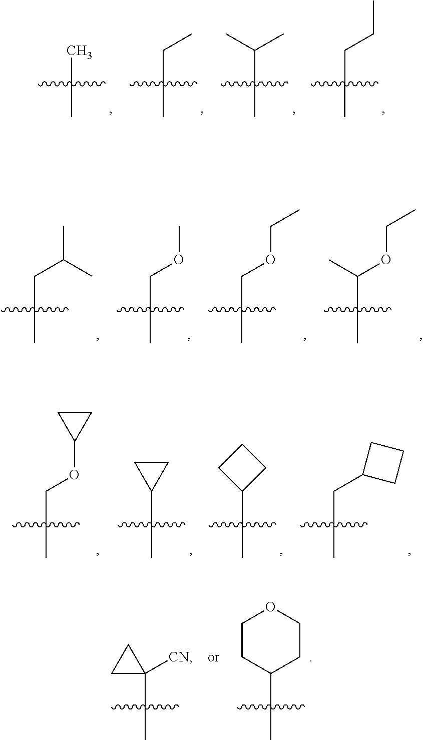

14. The compound of claim 1, or a pharmaceutically acceptable salt thereof, wherein R² is:

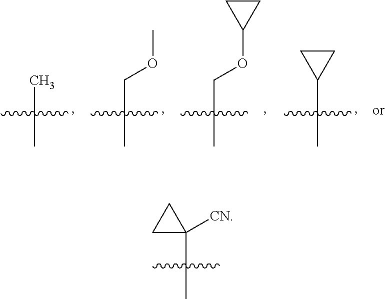

15. The compound of claim 1, or a pharmaceutically acceptable salt thereof, wherein R³ is: 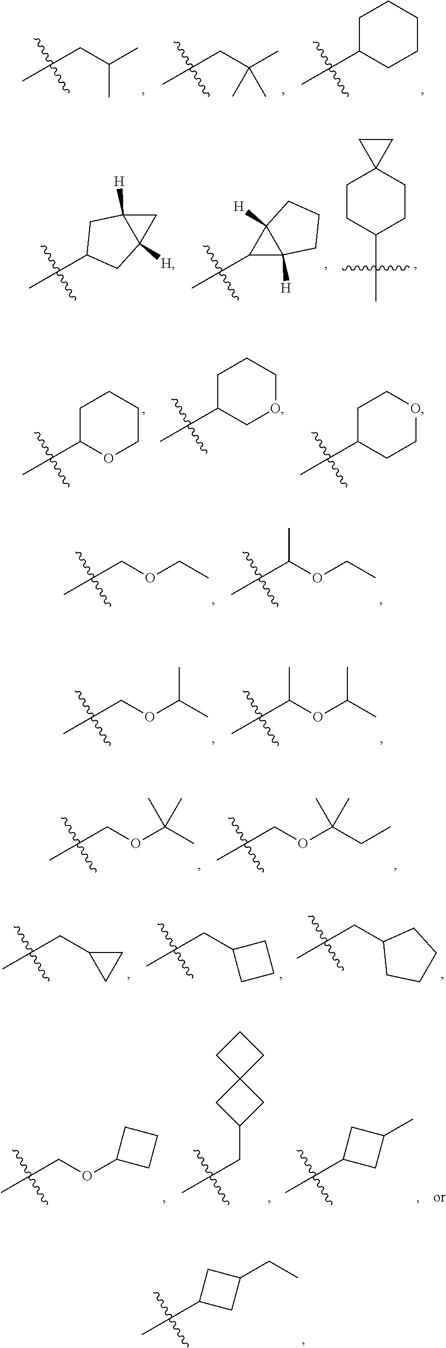
each of which is optionally substituted with one to three R^{3a} groups.

16. (canceled)

17. The compound of claim 1, or a pharmaceutically acceptable salt thereof, wherein R³ is:

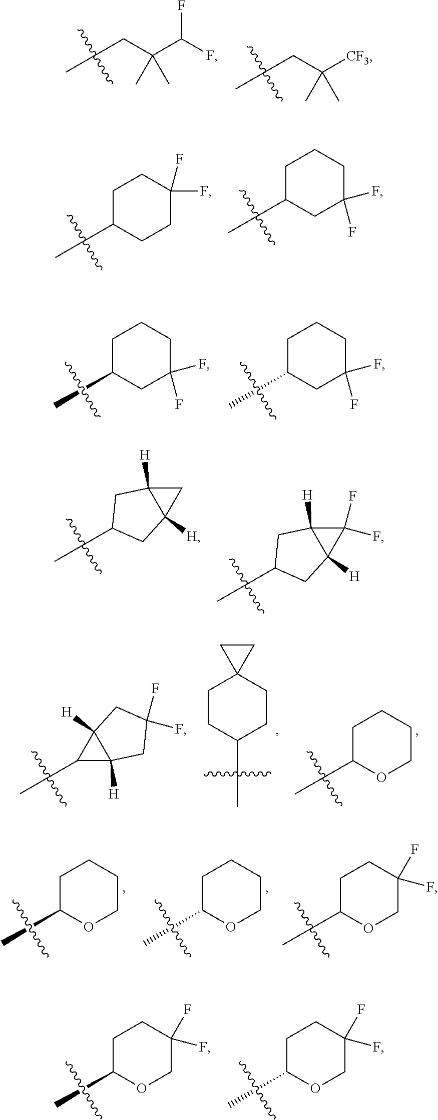

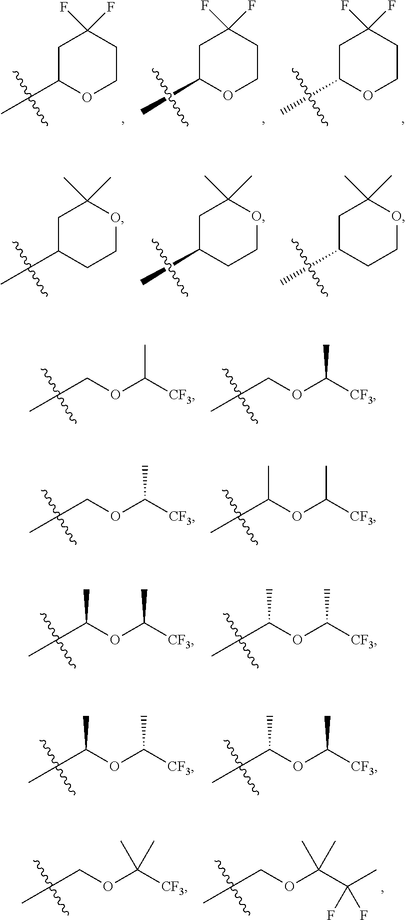

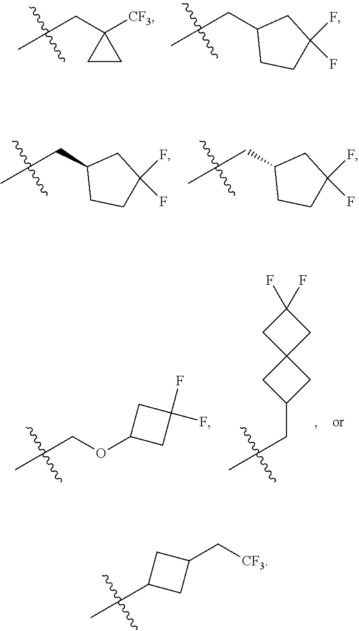

18-19. (canceled)

20. The compound of claim 1, or a pharmaceutically acceptable salt thereof, wherein R⁴ is isopropyl, —C₍₃₋₆₎cycloalkyl, —C₍₅₋₈₎polycycloalkyl, —C₍₁₋₂₎alkyl-C₍₃₋₅₎cycloalkyl, phenyl, or 5-membered heteroaryl, wherein the —C₍₃₋₆₎cycloalkyl, —C₍₅₋₈₎polycycloalkyl, and —C₍₁₋₂₎alkyl-C₍₃₋₅₎cycloalkyl are unsubstituted or substituted with one to three R^{4a} groups, wherein the phenyl is unsubstituted or substituted with one to three R^{4b} groups, and wherein the 5-membered heteroaryl is substituted with one to three R^{4c} groups.

21-30. (canceled)

31. The compound of claim 1, or a pharmaceutically acceptable salt thereof, wherein R⁴ is 5-membered heteroaryl, which is unsubstituted or substituted with one to three R^{4c} groups,
wherein each R^{4c} independently for each occurrence is fluorine, —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, —O—C₍₁₋₃₎alkyl, —C(O)NH₂, —CN, or —OH, wherein the —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —C₍₁₋₃₎alkyl-O—C₍₃₋₆₎cycloalkyl, and —O—C₍₁₋₃₎alkyl are unsubstituted or substituted with one to six substituents independently selected from the group consisting of fluorine, —OH, and —CN, or
wherein two R^{4c} groups attached to vicinal atoms on the same ring are combined with the atoms to which they are attached to form a C₍₃₋₆₎cycloalkyl or a 3- to 6-membered heterocyclyl.

32-37. (canceled)

38. The compound of claim 1, or a pharmaceutically acceptable salt thereof, wherein R^{4c} independently for each occurrence is fluorine, —C₍₁₋₆₎)alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, —O—C₍₁₋₃₎alkyl, —C(O)NH₂, —CN, or —OH, wherein the —C₍₁₋₆₎alkyl, —C₍₃₋₆₎cycloalkyl, —C₍₁₋₃₎alkyl-O—C₍₁₋₃₎alkyl, —C₍₁₋₃₎alkylC₍₃₋₆₎cycloalkyl, —C₍₁₋₃₎alkylC₍₅₋₈₎polycycloalkyl, and —O—C₍₁₋₃₎alkyl are unsubstituted or substituted with one to six substituents independently selected from the group consisting of fluorine, —OH, and —CN.

39-44. (canceled)

45. The compound of claim 1, or a pharmaceutically acceptable salt thereof, which is a compound of formula Ib:

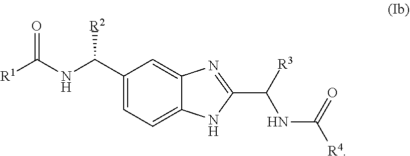

46-49. (canceled)

50. The compound of claim 1, or a pharmaceutically acceptable salt thereof, which is a compound of formula Ic-1a: 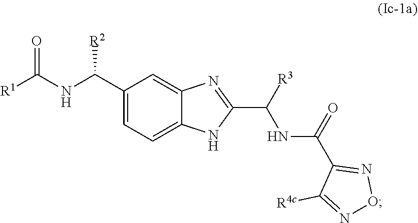
wherein:
R¹ is —C₍₁₋₃₎alkyl-C₍₃₋₆₎cycloalkyl, which is unsubstituted or substituted with one, two, or three fluorines;
R² is —C₍₁₋₃₎alkyl, cyclopropyl, cyclobutyl, or C₍₁₋₂₎alkyl-O—C₍₁₋₂₎alkyl, wherein the cyclopropyl is unsubstituted or substituted with one —CN;
R³ is —C₍₁₋₂₎alkyl-O—C₍₁₋₅₎alkyl, which is unsubstituted or substituted with one, two, or three substituents selected from the group consisting of fluorine, —CH₃, —CH₂F, —CHF₂, and —CF₃; and
R⁴, is —C₍₁₋₃₎alkyl or —C₍₃₋₄₎cycloalkyl.

51. (canceled)

52. The compound of claim 1, having a structure selected from the group consisting of: 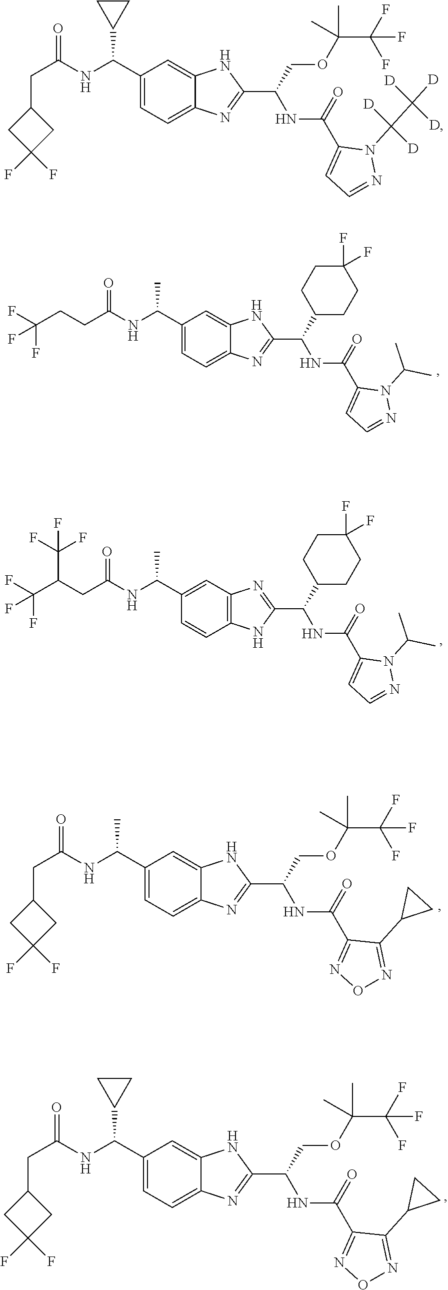 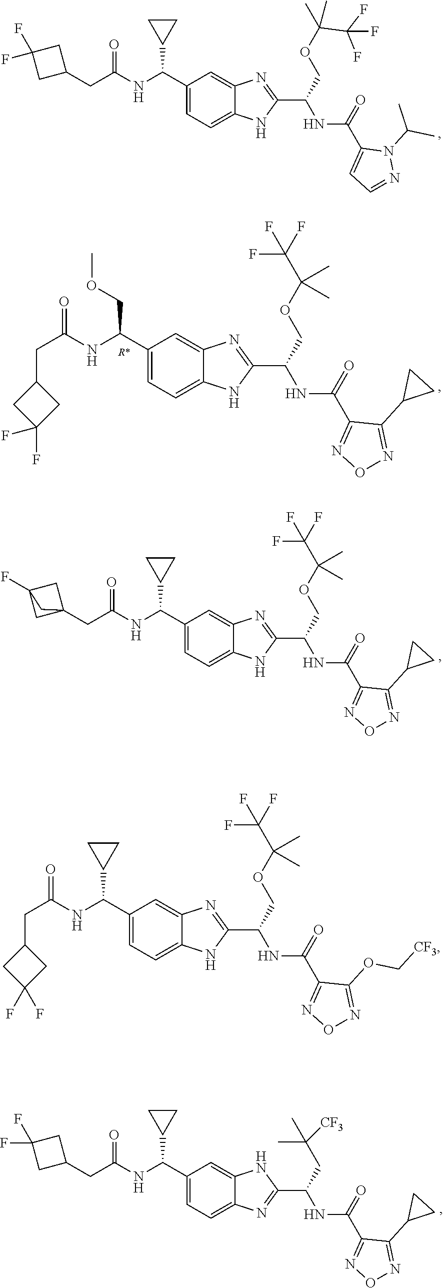 [SMILES: CC(C)n1nccc1C(=O)N[C@@H](COC(C)(C)C(F)(F)F)c1nc2cc([C@H](NC(=O)CC3CC(F)(F)C3)C3CC3)ccc2[nH]1 ; COC[C@H](NC(=O)CC1CC(F)(F)C1)c1ccc2[nH]c([C@H](COC(C)(C)C(F)(F)F)NC(=O)c3nonc3C3CC3)nc2c1 ; CC(C)(OC[C@H](NC(=O)c1nonc1C1CC1)c1nc2cc([C@H](NC(=O)CC34CC(F)(C3)C4)C3CC3)ccc2[nH]1)C(F)(F)F ; CC(C)(OC[C@H](NC(=O)c1nonc1OCC(F)(F)F)c1nc2cc([C@H](NC(=O)CC3CC(F)(F)C3)C3CC3)ccc2[nH]1)C(F)(F)F ; CC(C)(C[C@H](NC(=O)c1nonc1C1CC1)c1nc2ccc([C@H](NC(=O)CC3CC(F)(F)C3)C3CC3)cc2[nH]1)C(F)(F)F] 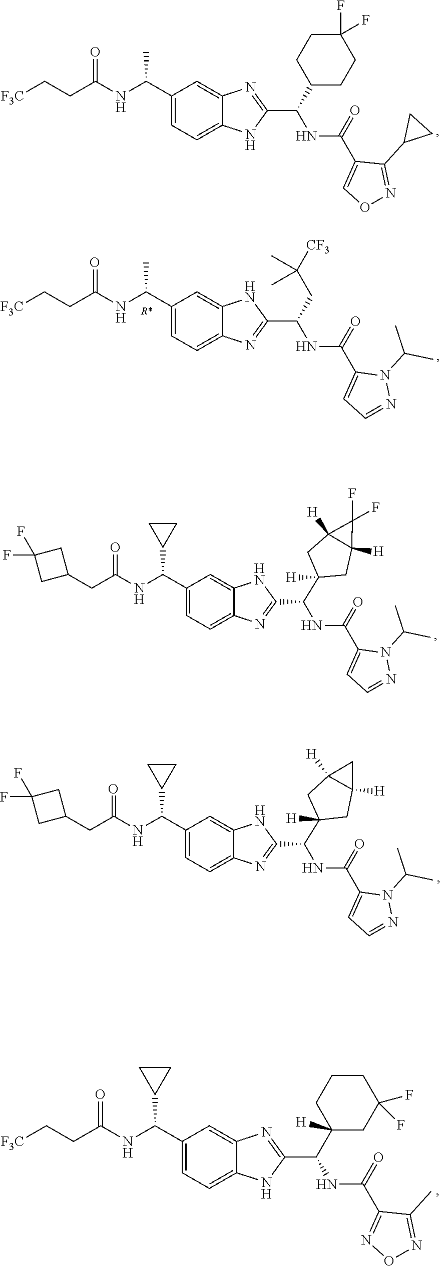 [SMILES: C[C@@H](NC(=O)CCC(F)(F)F)c1ccc2[nH]c([C@@H](NC(=O)c3conc3C3CC3)C3CCC(F)(F)CC3)nc2c1 ; CC(C)n1nccc1C(=O)N[C@@H](CC(C)(C)C(F)(F)F)c1nc2ccc([C@@H](C)NC(=O)CCC(F)(F)F)cc2[nH]1 ; CC(C)n1nccc1C(=O)N[C@H](c1nc2ccc([C@H](NC(=O)CC3CC(F)(F)C3)C3CC3)cc2[nH]1)[C@@H]1C[C@@H]2[C@H](C1)C2(F)F ; CC(C)n1nccc1C(=O)N[C@H](c1nc2ccc([C@H](NC(=O)CC3CC(F)(F)C3)C3CC3)cc2[nH]1)[C@@H]1C[C@H]2C[C@H]2C1 ; Cc1nonc1C(=O)N[C@H](c1nc2cc([C@H](NC(=O)CCC(F)(F)F)C3CC3)ccc2[nH]1)[C@@H]1CCCC(F)(F)C1] 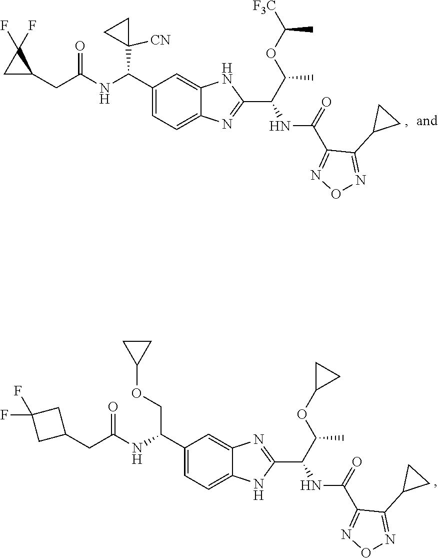 [SMILES: C[C@@H](O[C@H](C)C(F)(F)F)[C@H](NC(=O)c1nonc1C1CC1)c1nc2ccc([C@H](NC(=O)C[C@H]3CC3(F)F)C3(C#N)CC3)cc2[nH]1 ; C[C@@H](OC1CC1)[C@H](NC(=O)c1nonc1C1CC1)c1nc2cc([C@@H](COC3CC3)NC(=O)CC3CC(F)(F)C3)ccc2[nH]1]
or a pharmaceutically acceptable salt thereof.

53-69. (canceled)

70. A pharmaceutical composition, comprising a compound of claim 1, or a pharmaceutically acceptable salt thereof, and a pharmaceutically acceptable carrier.

71. (canceled)

72. The pharmaceutical composition of claim 70, or a pharmaceutically acceptable salt thereof, which is administered orally.

73. The pharmaceutical composition of claim 72, or a pharmaceutically acceptable salt thereof, which is administered as a tablet or a capsule.

74. (canceled)

75. A method for treating and/or ameliorating an IL-17A mediated inflammatory syndrome, disorder, or disease comprising administering to a subject in need thereof a therapeutically effective amount of a compound of claim 1, or a pharmaceutically acceptable salt thereof.

76. The method of claim 75, wherein the IL-17A mediated inflammatory syndrome, disorder, or disease is selected from the group consisting of psoriasis, psoriatic arthritis, rheumatoid arthritis, ankylosing spondylitis, hidradenitis suppurativa, bullous pemphigold, atopic dermatitis, vitiligo, multiple sclerosis, asthma, uveitis, chronic obstructive pulmonary disorder, multiple myeloma, and systemic lupus erythematosus.

77-95. (canceled)
